(19) United States
(12) Patent Application Publication
(10) Pub. No.: US 2026/0056201 A9
(43) Pub. Date: Feb. 26, 2026

(54) MARKERS SELECTIVELY DEREGULATED IN TUMOR-INFILTRATING REGULATORY T CELLS

(71) Applicant: CheckMab S.R.L., Milan (IT)

(72) Inventors: Sergio Abrignani, Rapolano Terme (IT); Massimiliano Pagani, Trescore Balneario (IT)

(73) Assignee: CheckMab S.R.L., Milan (IT)

(21) Appl. No.: 19/037,818

(22) Filed: Jan. 27, 2025

Related U.S. Application Data
(63) Continuation of application No. 18/295,965, filed on Apr. 5, 2023, now Pat. No. 12,247,982.
Application No. 18/295,965 is a continuation of application No. 16/301,805, now abandoned.

(30) Foreign Application Priority Data
May 16, 2016 (EP) 16169791.7
Nov. 14, 2016 (EP) 16198724.3

(51) Int. Cl. G01N 33/574; A61K 35/17; A61K 45/06; C07K 14/47; C07K 16/28; C07K 16/30; C12N 5/0783; C12N 15/113

(52) U.S. Cl. CPC G01N 33/57492; A61K 35/17; A61K 45/06; C07K 14/4748; C07K 16/2866; C07K 16/30; C12N 5/0637; C12N 15/1135; C12N 15/1138; C07K 2317/732; C07K 2317/76; C12N 2310/11; C12N 2310/14

(57) ABSTRACT

The present invention discloses a number of markers selectively deregulated in tumor-infiltrating regulatory T cells. The invention relates to molecules able to modulate the expression and/or function of at least one such marker for use in the prevention and/or treatment of the tumor. Preferably the molecule specifically binds to the marker and induces antibody-dependent cell-mediated cytotoxicity (ADCC). The invention further relates to a molecule able to modulate the expression and/or function of at least one such marker for use in a method for in vivo depleting tumor-infiltrating regulatory T cell in a subject, or for use in a method to enhance tumor immunity in a subject. Corresponding pharmaceutical compositions are also contemplated.

### RELATED APPLICATIONS

This application is a continuation of U.S. application Ser. No. 18/295,965, filed Apr. 5, 2023, which is a continuation of U.S. application Ser. No. 16/301,805, filed Nov. 15, 2018, which is a national stage filing under 35 U.S.C. § 371 of International Application Serial No. PCT/EP2017/061642, filed May 15, 2017, the contents of each of which is incorporated herein by reference in their entirety.

### REFERENCE TO AN ELECTRONIC SEQUENCE LISTING

The contents of the electronic sequence listing (C158570000US02-SEQ-JRV.xml; Size: 953,347 bytes; and Date of Creation: Jan. 24, 2025) is herein incorporated by reference in its entirety.

### FIELD OF THE INVENTION

The present invention relates to a molecule able to modulate the expression and/or function of at least one marker that is selectively deregulated in tumor-infiltrating regulatory T cell or to a molecule capable of specifically binding to at least one marker that is selectively deregulated in tumor-infiltrating regulatory T cell and inducing antibody-dependent cell-mediated cytotoxicity (ADCC) for use in the prevention and/or treatment of cancer or for use in a method for in vivo depleting tumor-infiltrating regulatory T cell in a subject or for use in a method to enhance tumor immunity in a subject and relative pharmaceutical composition.

### BACKGROUND OF THE INVENTION

The combination of genetic mutations and epigenetic modifications that are peculiar to all tumors generate antigens that T and B lymphocytes can use to specifically recognize tumor cells (Jamal-Hanjani et al., 2013). It is increasingly clear that T lymphocytes recognizing tumor derived peptides presented by major histocompatibility complex (MHC) molecules play a central role in immunotherapy and in conventional chemo-radiotherapy of cancer (Galluzzi et al., 2015). In fact, anti-tumor T cell responses arise in cancer patients but are disabled upon tumor progression by suppressive mechanisms triggered by the interplay between malignant cells and the tumor microenvironment (Munn and Bronte, 2015). The tumor-dependent immunosuppressive mechanisms depend on the integrated action of infiltrating leukocytes and lymphocytes that upregulate a range of modulatory molecules, collectively called immune checkpoints, whose function is only partially characterized (Pardoll, 2012). Therefore, the search for agonists of co-stimulatory complexes or antagonists of inhibitory molecules to potentiate antigen-specific T cell responses is a primary goal of current anti-tumor research (Sharma and Allison, 2015; Zitvogel et al., 2013). Indeed, clinical trials have unequivocally shown that the blockade of immune checkpoints unleashes the spontaneous anti-tumor immune responses in such a powerful way that it has created a paradigm shift in cancer therapy (Sledzinska et al., 2015; Topalian et al., 2015).

Amongst the immune checkpoints targeted by blocking strategies, CTLA-4 has been one of the first to be translated into therapeutic applications.

Anti-CTLA-4 monoclonal antibodies (mAb) showed remarkable success in metastatic melanoma, and more recently in non-small-cell lung cancer, prostate cancer, renal cell carcinoma, urothelial carcinoma and ovarian cancer (Carthon et al., 2010; Hodi et al., 2010; van den Eertwegh et al., 2012; Yang et al., 2007). However, the fraction of patients that do not respond remains high, prompting a deeper investigation of the mechanisms underpinning the modulation of immune responses by tumors. Recent experimental evidence showed that anti-CTLA-4 mAb efficacy depends on FcγR mediated depletion of CD4⁺ regulatory T cells (Treg cells) within the tumor microenvironment (Peggs et al., 2009; Selby et al., 2013; Simpson et al., 2013; Twyman-Saint Victor et al., 2015). Treg cells, which are physiologically engaged in the maintenance of immunological self-tolerance and immune homeostasis (Josefowicz et al., 2012; Sakaguchi et al., 2008), are potent suppressors of effector cells and are found at high frequencies in various types of cancers (Fridman et al., 2012; Nishikawa and Sakaguchi, 2010). Interestingly, Treg cells adapt their transcriptional program to the various cytokines to which they are exposed in the inflammatory milieu (Campbell and Koch, 2011). This versatility is controlled by transcription factors generally associated with the differentiation of other effector CD4+ T cell subsets, resulting in various Treg cell populations with unique features and immunomodulatory functions (Duhen et al., 2012; Geginat et al., 2014). Moreover, Treg cells infiltrating non-lymphoid tissues are reported to exhibit unique phenotypes and transcriptional signatures, as they can display functions beyond their well-established suppressive roles, such as metabolic modulation in adipose tissue (Cipolletta et al., 2012) or regulation of tissue repair in skeletal muscle (Burzyn et al., 2013) and in lung tissue (Arpaia et al., 2015).

Treg cells depletion has been reported to increase anti-tumor specific immune responses and to reduce tumor burden (Marabelle et al., 2013; Teng et al., 2010; Walter et al., 2012).

Although promising clinical results have been achieved with Treg cell depleting strategies, some relevant issues are to be addressed, for a safer, more effective and wider clinical application of these therapies. First, severe autoimmunity can occur following systemic Treg cells depletion (Nishikawa and Sakaguchi, 2010), which could be avoided if selective depletion of tumor infiltrating Treg cells were feasible. A second issue concerns the specificity of targeting, indeed Treg cells share with effector lymphocytes most of the molecules targeted for therapy, which can possibly deplete also the tumor-specific effector cells. Therefore, the molecular characterization of Treg cells at different tumor sites should help to better define therapeutic targets through a better description of their signature molecules and of the network that regulates Treg cell functions in the tumor microenvironment.

Non-small-cell lung cancer (NSCLC) and colorectal cancer (CRC) are the two most frequent cancers in both genders (Torre et al., 2015). NSCLC has the worst prognosis due to its high mortality rate even in early stages. Although CRC survival rate is highly dependent on the tumor stage at diagnosis, about 50% of patients will progress to metastatic cancer (Gonzalez-Pons and Cruz-Correa, 2015). Both tumors have been targeted with therapies based on monoclonal antibodies to checkpoint inhibitors, but the outcomes were different. While remarkable clinical success has been obtained in NSCLC, evidence of durable response in CRC is scarce with the exception of mismatch repair-deficient CRC lesions (Jacobs et al., 2015; Kroemer et al., 2015; Le et al., 2015).

Then there is still need for agents that target tumor infiltrating Treg cells for the treatment and/or prevention of cancer.

### SUMMARY OF THE INVENTION

Tumor-infiltrating regulatory T lymphocytes (Treg) can suppress effector T cells specific for tumor antigens. Since new anti-cancer immunotherapies aim at unleashing effector T cells by targeting immune-checkpoints, deeper molecular definitions of tumor-infiltrating-lymphocytes could offer new therapeutic opportunities. Transcriptomes of T helper 1(Th1), Th17 and Treg cells infiltrating colorectal or non-small-cell lung cancers were compared to transcriptomes of the same subsets from normal tissues, and validated at the single cell level. The inventors found tumor-infiltrating Treg cells are highly suppressive, upregulate several immune-checkpoints, and express on the cell surface specific signature molecules such as interleukin-1 receptor 2 (IL1R2), programmed death (PD)-1 Ligand1, PD-1 Ligand2, and CCR8 chemokine which were not previously described on Treg cells. Remarkably, high expression in whole tumor samples of Treg signature genes, such as LAYN, MAGEH1 or CCR8, correlated with poor prognosis. The invention provides new insights into the molecular identity and functions of human tumor-infiltrating Treg cells, and define new potential targets for tumor immunotherapy.

In the present invention, the inventors provide a comprehensive transcriptome analysis of human CD4⁺ Treg cells and effector cells (Th1 and Th17) infiltrating NSCLC or CRC and their matched normal tissues.

Inventors defined molecular signatures of tumor-infiltrating Treg cells in these two cancer types and confirmed the relevance of these signatures by single-cell analyses. These data could help a better understanding of Treg functional role at tumor sites and pave the way to the identification of therapeutic targets for more specific and safer modulation of Treg cells in cancer therapy.

The inventors' findings provide new insights on the inhibitory mechanisms of Treg cells and offer precise targets for cancer immunotherapy.

Then the present invention provides a molecule able to modulate the expression and/or function of at least one marker that is selectively deregulated in tumor-infiltrating regulatory T cells for use in the prevention and/or treatment of said tumor.

Preferably, the molecule according to the invention is capable of specifically binding to said at least one marker and inducing antibody-dependent cell-mediated cytotoxicity (ADCC).

Said molecule is preferably able to selectively deplete tumor-infiltrating regulatory T cells.

Said molecule is preferably selected from the group consisting of:
a) an antibody or a fragment thereof;
b) a polypeptide;
c) a small molecule;
d) a polynucleotide coding for said antibody or polypeptide or a functional derivative thereof;
e) a polynucleotide, such as antisense construct, antisense oligonucleotide, RNA interference construct or siRNA,
e) a vector comprising or expressing the polynucleotide as defined in d) or e);
f) a host cell genetically engineered expressing said polypeptide or antibody or comprising the polynucleotide as defined in d) or e).

Preferably, the marker is selected from the group consisting of at least one marker disclosed in the following Table VIII.

TABLE VIII

| MARKER | | ENTREZ_ID |
|---|---|---|
| NAME | ENSEMBL_release87 | release108 |
| FUCA2 | ENSG00000001036 | 2519 |
| ICA1 | ENSG00000003147 | 3382 |
| TTC22 | ENSG00000006555 | 55001 |
| COX10 | ENSG00000006695 | 1352 |
| IL32 | ENSG00000008517 | 9235 |
| ETV7 | ENSG00000010030 | 51513 |
| ATP2C1 | ENSG00000017260 | 27032 |
| FAS | ENSG00000026103 | 355 |
| ARNTL2 | ENSG00000029153 | 56938 |
| IKZF2 | ENSG00000030419 | 22807 |
| PEX3 | ENSG00000034693 | 8504 |
| MAT2B | ENSG00000038274 | 27430 |
| TSPAN17 | ENSG00000048140 | 26262 |
| COL9A2 | ENSG00000049089 | 1298 |
| TNFRSF9 | ENSG00000049249 | 3604 |
| FOXP3 | ENSG00000049768 | 50943 |
| NFE2L3 | ENSG00000050344 | 9603 |
| LIMA1 | ENSG00000050405 | 51474 |
| TNIP3 | ENSG00000050730 | 79931 |
| LY75 | ENSG00000054219 | 4065 |
| ZNF280C | ENSG00000056277 | 55609 |
| YIPF1 | ENSG00000058799 | 54432 |
| NFYC | ENSG00000066136 | 4802 |
| ISOC1 | ENSG00000066583 | 51015 |
| PHKA1 | ENSG00000067177 | 5255 |
| ACSL4 | ENSG00000068366 | 2182 |
| MAST4 | ENSG00000069020 | 375449 |
| LMCD1 | ENSG00000071282 | 29995 |
| TFRC | ENSG00000072274 | 7037 |
| PANX2 | ENSG00000073150 | 56666 |
| FNDC3B | ENSG00000075420 | 64778 |
| REXO2 | ENSG00000076043 | 25996 |
| TP73 | ENSG00000078900 | 7161 |
| LXN | ENSG00000079257 | 56925 |
| CEACAM1 | ENSG00000079385 | 634 |
| IL12RB2 | ENSG00000081985 | 3595 |
| GSK3B | ENSG00000082701 | 2932 |
| TDRD3 | ENSG00000083544 | 81550 |
| RRAGB | ENSG00000083750 | 10325 |
| STARD7 | ENSG00000084090 | 56910 |
| SSH1 | ENSG00000084112 | 54434 |
| NCOA1 | ENSG00000084676 | 8648 |
| MGST2 | ENSG00000085871 | 4258 |
| ACOX3 | ENSG00000087008 | 8310 |
| AURKA | ENSG00000087586 | 6790 |
| TPX2 | ENSG00000088325 | 22974 |
| ANKRD10 | ENSG00000088448 | 55608 |
| FKBP1A | ENSG00000088832 | 2280 |
| SIRPG | ENSG00000089012 | 55423 |
| BIRC5 | ENSG00000089685 | 332 |
| RGS1 | ENSG00000090104 | 5996 |
| DPYSL2 | ENSG00000092964 | 1808 |
| WHRN | ENSG00000095397 | 25861 |
| CENPM | ENSG00000100162 | 79019 |
| SEPT3 | ENSG00000100167 | 55964 |
| NCF4 | ENSG00000100365 | 4689 |
| CSF2RB | ENSG00000100368 | 1439 |
| IL2RB | ENSG00000100385 | 3560 |
| CNIH1 | ENSG00000100528 | 10175 |
| ZMYND8 | ENSG00000101040 | 23613 |
| MAP1LC3A | ENSG00000101460 | 84557 |
| PIGU | ENSG00000101464 | 128869 |
| NXT2 | ENSG00000101888 | 55916 |
| SMS | ENSG00000102172 | 6611 |
| NDFIP2 | ENSG00000102471 | 54602 |
| ACP5 | ENSG00000102575 | 54 |
| NFAT5 | ENSG00000102908 | 10725 |
| CYB5B | ENSG00000103018 | 80777 |
| IL21R | ENSG00000103522 | 50615 |
| LAPTM4B | ENSG00000104341 | 55353 |
| IL7 | ENSG00000104432 | 3574 |
| NCALD | ENSG00000104490 | 83988 |
| ERI1 | ENSG00000104626 | 90459 |
| EBI3 | ENSG00000105246 | 10148 |
| PLA2G4C | ENSG00000105499 | 8605 |
| CDK6 | ENSG00000105810 | 1021 |
| HOXA1 | ENSG00000105991 | 3198 |
| GLCCI1 | ENSG00000106415 | 113263 |
| MINPP1 | ENSG00000107789 | 9562 |
| ACTA2 | ENSG00000107796 | 59 |
| WSB1 | ENSG00000109046 | 26118 |
| CLNK | ENSG00000109684 | 116449 |
| HTATIP2 | ENSG00000109854 | 10553 |
| CTSC | ENSG00000109861 | 1075 |
| VWA5A | ENSG00000110002 | 4013 |
| DCPS | ENSG00000110063 | 28960 |
| SLC35F2 | ENSG00000110660 | 54733 |
| FOXM1 | ENSG00000111206 | 2305 |
| RAD51AP1 | ENSG00000111247 | 10635 |
| RASAL1 | ENSG00000111344 | 8437 |
| VDR | ENSG00000111424 | 7421 |
| FAM184A | ENSG00000111879 | 79632 |
| DNPH1 | ENSG00000112667 | 10591 |
| KIF20A | ENSG00000112984 | 10112 |
| SEC24A | ENSG00000113615 | 10802 |
| KAT2B | ENSG00000114166 | 8850 |
| PPM1G | ENSG00000115241 | 5496 |
| IL1R2 | ENSG00000115590 | 7850 |
| IL1R1 | ENSG00000115594 | 3554 |
| IL1RL2 | ENSG00000115598 | 8808 |
| IL1RL1 | ENSG00000115602 | 9173 |
| UXS1 | ENSG00000115652 | 80146 |
| SLC25A12 | ENSG00000115840 | 8604 |
| THADA | ENSG00000115970 | 63892 |
| PARK7 | ENSG00000116288 | 11315 |
| LEPR | ENSG00000116678 | 3953 |
| GADD45A | ENSG00000116717 | 1647 |
| KIF14 | ENSG00000118193 | 9928 |
| MREG | ENSG00000118242 | 55686 |
| HSDL2 | ENSG00000119471 | 84263 |
| FLVCR2 | ENSG00000119686 | 55640 |
| CD274 | ENSG00000120217 | 29126 |
| SOCS2 | ENSG00000120833 | 8835 |
| TNFRSF8 | ENSG00000120949 | 943 |
| RDH10 | ENSG00000121039 | 157506 |
| LAX1 | ENSG00000122188 | 54900 |
| TWIST1 | ENSG00000122691 | 7291 |
| ZWINT | ENSG00000122952 | 11130 |
| CIT | ENSG00000122966 | 11113 |
| ACOT9 | ENSG00000123130 | 23597 |
| IKZF4 | ENSG00000123411 | 64375 |
| HJURP | ENSG00000123485 | 55355 |
| METTL8 | ENSG00000123600 | 79828 |
| TOX2 | ENSG00000124191 | 84969 |
| GTSF1L | ENSG00000124196 | 149699 |
| SOX4 | ENSG00000124766 | 6659 |
| TM9SF2 | ENSG00000125304 | 9375 |
| HS3ST3B1 | ENSG00000125430 | 9953 |
| EML2 | ENSG00000125746 | 24139 |
| MGME1 | ENSG00000125871 | 92667 |
| IGFLR1 | ENSG00000126246 | 79713 |
| DLGAP5 | ENSG00000126787 | 9787 |
| HIVEP3 | ENSG00000127124 | 59269 |
| LRRC61 | ENSG00000127399 | 65999 |
| TST | ENSG00000128311 | 7263 |
| STRIP2 | ENSG00000128578 | 57464 |
| MYO5C | ENSG00000128833 | 55930 |
| FOXA1 | ENSG00000129514 | 3169 |
| ITFG1 | ENSG00000129636 | 81533 |
| KLHDC7B | ENSG00000130487 | 113730 |
| TRAF3 | ENSG00000131323 | 7187 |
| MCCC2 | ENSG00000131844 | 64087 |
| GRSF1 | ENSG00000132463 | 2926 |
| SYT11 | ENSG00000132718 | 23208 |
| SLC41A1 | ENSG00000133065 | 254428 |
| ATP13A3 | ENSG00000133657 | 79572 |
| MICAL2 | ENSG00000133816 | 9645 |
| IL2RA | ENSG00000134460 | 3559 |
| CABLES1 | ENSG00000134508 | 91768 |
| RFK | ENSG00000135002 | 55312 |
| HAVCR2 | ENSG00000135077 | 84868 |
| CGA | ENSG00000135346 | 1081 |
| FAIM2 | ENSG00000135472 | 23017 |
| EGLN1 | ENSG00000135766 | 54583 |
| ARHGEF4 | ENSG00000136002 | 50649 |
| SLC41A2 | ENSG00000136052 | 84102 |
| FLNB | ENSG00000136068 | 2317 |
| RCBTB1 | ENSG00000136144 | 55213 |
| TMOD1 | ENSG00000136842 | 7111 |
| TPMT | ENSG00000137364 | 7172 |
| CASP1 | ENSG00000137752 | 834 |
| NUSAP1 | ENSG00000137804 | 51203 |
| ADAM10 | ENSG00000137845 | 102 |
| ZNF280D | ENSG00000137871 | 54816 |
| HADHB | ENSG00000138029 | 3032 |
| CEP55 | ENSG00000138180 | 55165 |
| ENTPD1 | ENSG00000138185 | 953 |
| NAB1 | ENSG00000138386 | 4664 |
| HECW2 | ENSG00000138411 | 57520 |
| CD27 | ENSG00000139193 | 939 |
| CDH24 | ENSG00000139880 | 64403 |
| RAB15 | ENSG00000139998 | 376267 |
| ETFA | ENSG00000140374 | 2108 |
| KSR1 | ENSG00000141068 | 8844 |
| PCTP | ENSG00000141179 | 58488 |
| SECTM1 | ENSG00000141574 | 6398 |
| EVA1B | ENSG00000142694 | 55194 |
| WDTC1 | ENSG00000142784 | 23038 |
| CTTNBP2NL | ENSG00000143079 | 55917 |
| CASQ1 | ENSG00000143318 | 844 |
| SNAP47 | ENSG00000143740 | 116841 |
| STAC | ENSG00000144681 | 6769 |
| ARL6IP5 | ENSG00000144746 | 10550 |
| ADPRH | ENSG00000144843 | 141 |
| PAM | ENSG00000145730 | 5066 |
| RNF145 | ENSG00000145860 | 153830 |
| TTBK1 | ENSG00000146216 | 84630 |
| TMEM140 | ENSG00000146859 | 55281 |
| CHST7 | ENSG00000147119 | 56548 |
| CHRNA6 | ENSG00000147434 | 8973 |
| MKI67 | ENSG00000148773 | 4288 |
| PTPRJ | ENSG00000149177 | 5795 |
| ZC3H12C | ENSG00000149289 | 85463 |
| NCAM1 | ENSG00000149294 | 4684 |
| INPP1 | ENSG00000151689 | 3628 |
| JAKMIP1 | ENSG00000152969 | 152789 |
| GTF3C6 | ENSG00000155115 | 112495 |
| RHOC | ENSG00000155366 | 389 |
| SLC16A1 | ENSG00000155380 | 6566 |
| BATF | ENSG00000156127 | 10538 |
| CXCL13 | ENSG00000156234 | 10563 |
| SH3RF2 | ENSG00000156463 | 153769 |
| NPTN | ENSG00000156642 | 27020 |
| CCNB2 | ENSG00000157456 | 9133 |
| RNF207 | ENSG00000158286 | 388591 |
| AHCYL2 | ENSG00000158467 | 23382 |
| PTGIR | ENSG00000160013 | 5739 |
| CALM3 | ENSG00000160014 | 808 |
| TMPRSS3 | ENSG00000160183 | 64699 |
| FCRL3 | ENSG00000160856 | 115352 |
| PAQR4 | ENSG00000162073 | 124222 |
| ZG16B | ENSG00000162078 | 124220 |
| JAK1 | ENSG00000162434 | 3716 |
| DIRAS3 | ENSG00000162595 | 9077 |
| ACTG2 | ENSG00000163017 | 72 |
| SGPP2 | ENSG00000163082 | 130367 |
| NEURL3 | ENSG00000163121 | 93082 |
| CTLA4 | ENSG00000163599 | 1493 |
| ICOS | ENSG00000163600 | 29851 |
| RYBP | ENSG00000163602 | 23429 |
| KIF15 | ENSG00000163808 | 56992 |
| TMEM184C | ENSG00000164168 | 55751 |
| C5orf63 | ENSG00000164241 | 401207 |
| PTTG1 | ENSG00000164611 | 9232 |
| MELK | ENSG00000165304 | 9833 |
| FAAH2 | ENSG00000165591 | 158584 |
| PRDX3 | ENSG00000165672 | 10935 |
| HPRT1 | ENSG00000165704 | 3251 |
| CACNB2 | ENSG00000165995 | 783 |
| TPP1 | ENSG00000166340 | 1200 |
| AKIP1 | ENSG00000166452 | 56672 |
| ACAA2 | ENSG00000167315 | 10449 |
| GNG8 | ENSG00000167414 | 94235 |
| GNG4 | ENSG00000168243 | 2786 |
| CX3CR1 | ENSG00000168329 | 1524 |
| AHCYL1 | ENSG00000168710 | 10768 |
| TSPAN5 | ENSG00000168785 | 10098 |
| PGM2 | ENSG00000169299 | 55276 |
| CRADD | ENSG00000169372 | 8738 |
| UGP2 | ENSG00000169764 | 7360 |
| ZNF282 | ENSG00000170265 | 8427 |
| GLB1 | ENSG00000170266 | 2720 |
| SMAD1 | ENSG00000170365 | 4086 |
| SPATA24 | ENSG00000170469 | 202051 |
| PRKCDBP | ENSG00000170955 | 112464 |
| TADA3 | ENSG00000171148 | 10474 |
| RBKS | ENSG00000171174 | 64080 |
| NETO2 | ENSG00000171208 | 81831 |
| LRG1 | ENSG00000171236 | 116844 |
| FAM98B | ENSG00000171262 | 283742 |
| CHST11 | ENSG00000171310 | 50515 |
| ECEL1 | ENSG00000171551 | 9427 |
| BCL2L1 | ENSG00000171552 | 598 |
| MALT1 | ENSG00000172175 | 10892 |
| ZMAT3 | ENSG00000172667 | 64393 |
| CORO1B | ENSG00000172725 | 57175 |
| CYP7B1 | ENSG00000172817 | 9420 |
| HPSE | ENSG00000173083 | 10855 |
| VANGL1 | ENSG00000173218 | 81839 |
| GLRX | ENSG00000173221 | 2745 |
| TRIB1 | ENSG00000173334 | 10221 |
| CD7 | ENSG00000173762 | 924 |
| HAP1 | ENSG00000173805 | 9001 |
| FBXO45 | ENSG00000174013 | 200933 |
| CHST2 | ENSG00000175040 | 9435 |
| RMI2 | ENSG00000175643 | 116028 |
| SLC35E3 | ENSG00000175782 | 55508 |
| ZBTB38 | ENSG00000177311 | 253461 |
| ZBED2 | ENSG00000177494 | 79413 |
| PARD6G | ENSG00000178184 | 84552 |
| GLDC | ENSG00000178445 | 2731 |
| AKAP5 | ENSG00000179841 | 9495 |
| CCR8 | ENSG00000179934 | 1237 |
| PAK2 | ENSG00000180370 | 5062 |
| YIPF6 | ENSG00000181704 | 286451 |
| TIGIT | ENSG00000181847 | 201633 |
| CREB3L2 | ENSG00000182158 | 64764 |
| XKRX | ENSG00000182489 | 402415 |
| CADM1 | ENSG00000182985 | 23705 |
| LHFP | ENSG00000183722 | 10186 |
| CSF1 | ENSG00000184371 | 1435 |
| PTP4A3 | ENSG00000184489 | 11156 |
| CDCA2 | ENSG00000184661 | 157313 |
| OSBP2 | ENSG00000184792 | 23762 |
| METTL7A | ENSG00000185432 | 25840 |
| SPATC1 | ENSG00000186583 | 375686 |
| TNFRSF4 | ENSG00000186827 | 7293 |
| TNFRSF18 | ENSG00000186891 | 8784 |
| TMPRSS6 | ENSG00000187045 | 164656 |
| GCNT1 | ENSG00000187210 | 2650 |
| MAGEH1 | ENSG00000187601 | 28986 |
| NHS | ENSG00000188158 | 4810 |
| IL17REL | ENSG00000188263 | 400935 |
| ADAT2 | ENSG00000189007 | 134637 |
| NEMP2 | ENSG00000189362 | 100131211 |
| SPATS2L | ENSG00000196141 | 26010 |
| NTNG2 | ENSG00000196358 | 84628 |
| MYL6B | ENSG00000196465 | 140465 |
| ARHGEF12 | ENSG00000196914 | 23365 |
| MAP3K5 | ENSG00000197442 | 4217 |
| PDGFA | ENSG00000197461 | 5154 |
| PDCD1LG2 | ENSG00000197646 | 80380 |
| TOR4A | ENSG00000198113 | 54863 |
| HIBCH | ENSG00000198130 | 26275 |
| ZNF334 | ENSG00000198185 | 55713 |
| NTRK1 | ENSG00000198400 | 4914 |
| TMA16 | ENSG00000198498 | 55319 |
| WDHD1 | ENSG00000198554 | 11169 |
| FAM19A2 | ENSG00000198673 | 338811 |
| F5 | ENSG00000198734 | 2153 |
| GK | ENSG00000198814 | 2710 |
| INPP5F | ENSG00000198825 | 22876 |
| LAYN | ENSG00000204381 | 143903 |
| CARD16 | ENSG00000204397 | 114769 |
| TBC1D8 | ENSG00000204634 | 11138 |
| CD177 | ENSG00000204936 | 57126 |
| LEPROT | ENSG00000213625 | 54741 |
| SEC14L6 | ENSG00000214491 | 730005 |
| TRIM16 | ENSG00000221926 | 10626 |
| LTA | ENSG00000226979 | 4049 |
| PROB1 | ENSG00000228672 | 389333 |
| AF165138.7 | ENSG00000243440 | NA |
| USP51 | ENSG00000247746 | 158880 |
| CARD17 | ENSG00000255221 | 440068 |
| DOC2B | ENSG00000272636 | 8447 |
| C17orf96 | ENSG00000273604 | 100170841 |
| SSTR3 | ENSG00000278195 | 6753 |
| AC019206.1 | ENSG00000279229 | NA |

wherein each of said marker name is characterized by “Ensembl gene id” and includes all of therein disclosed isoform protein sequences.

Each gene of table VIII is characterized by its Ensembl Gene accession number (ENSG), retrievable in the public database EnsEMBL (http://www.ensembi.orcl) and by its Entrez Gene ID, retrievable in the public database NCBI (https://www.ncbi.nim.nih.gov/), if present.

Preferably the marker is selected from the group consisting of: a transmembrane protein, a cytokine, an epigenetic factor, a kinase phosphatase or a transcription factor.

More preferably, the marker is a transmembrane protein selected from the group of SEQ ID NO:1-661, even more preferably, the marker is selected from the group consisting of: LAYN (SEQ ID NOs:1-9), CCR8 (SEQ ID Nos:10-11), IL21R (SEQ ID Nos: 12-14), IL1 R2 (SEQ ID Nos:206-209), LY75 (SEQ ID NO: 78), SIRPG (SEQ ID Nos:122-126), CD177 (SEQ ID Nos:651-653), CD7 (SEQ ID Nos:549-554), FCRL3 (SEQ ID Nos:452-457), CADM1 (SEQ ID Nos: 570-583), NTNG2 (SEQ ID Nos:621-622), CSF2RB (SEQ ID Nos:134-137), SECTMI (SEQ ID Nos: 349-356), TSPAN5 (SEQ ID Nos:497-503), TMPRSS3 (SEQ ID Nos:448-451), TMPRSS6 (SEQ ID Nos:605-611), METTL7A (SEQ ID Nos:600-604), THADA (SEQ ID Nos: 237), NDFIP2 (SEQ ID Nos:148-151), CHRNA6 (SEQ ID Nos:392-394), or from the group consisting of:

LAYN (SEQ ID NOS: 1-9
[>ENSG00000204381_ENST00000375614_ENSP00000364764_LAYN
MRPGTALQAVLLAVLLVGLRAATGRLLSGQPVCRGGTQRPCYKVIYFHDTSRRLNFEEAKEACR
RDGGQLVSIESEDEQKLIEKFIENLLPSDGDFWIGLRRREEKQSNSTACQDLYAWTDGSISQFRN
WYVDEPSCGSEVCVVMYHQPSAPAGIGGPYMFQWNDDRCNMKNNFICKYSDEKPAVPSREAE
GEETELTTPVLPEETQEEDAKKTFKESREAALNLAYILIPSIPLLLLLVVTTVVCWVWICRKRKRE
QPDPSTKKQHTIWPSPHQGNSPDLEVYNVIRKQSEADLAETRPDLKNISFRVCSGEATPDDMSCD
YDNMAVNPSESGFVTLVSVESGFVTNDIYEFSPDQMGRSKESGWVENEIYGY* (SEQ ID NO: 1)
>ENSG00000204381_ENST00000375615_ENSP00000364765_LAYN
MRPGTALQAVLLAVLLVGLRAATGRLLSASDLDLRGGQPVCRGGTQRPCYKVIYFHDTSRRLNF
EEAKEACRRDGGQLVSIESEDEQKLIEKFIENLLPSDGDFWIGLRRREEKQSNSTACQDLYAWTD
GSISQFRNWYVDEPSCGSEVCVVMYHQPSAPAGIGGPYMFQWNDDRCNMKNNFICKYSDEKPA
VPSREAEGEETELTTPVLPEETQEEDAKKTFKESREAALNLAYILIPSIPLLLLLVVTTVVCWVWIC
RKRKREQPDPSTKKQHTIWPSPHQGNSPDLEVYNVIRKQSEADLAETRPDLKNISFRVCSGEATP
DDMSCDYDNMAVNPSESGFVTLVSVESGFVTNDIYEFSPDQMGRSKESGWVENEIYGY*
(SEQ ID NO: 2)
>ENSG00000204381_ENST00000436913_ENSP00000392942_LAYN
MVTSGLGSGGVRRNKAIAQPARTFMLGLMAAYHNLEKPAVPSREAEGEETELTTPVLPEETQEE
DAKKTFKESREAALNLAYILIPSIPLLLLLVVTTVVCWVWICRKRKREQPDPSTKKQHTIWPSPHQ
GNSPDLEVYNVIRKQSEADLAETRPDLKNISFRVCSGEATPDDMSCDYDNMAVNPSESGFVTLV
SVESGFVTNDIYEFSPDQMGRSKESGWVENEIYGY* (SEQ ID NO: 3)
>ENSG00000204381_ENST00000525126_ENSP00000434328_LAYN
MRPGTALQAVLLAVLLVGLRAATGRLLSASDLDLRGGQPVCRGGTQRPCYKVIYFHDTSRRLNF
EEAKEACRRDGGQLVSIESEDEQKLIEKFIENLLPSDGDFWIGLRRREEKQSNSTACQDLYAWTD
GSISQFRNWYVDEPSCGSEVCVVMYHQPSAPAGIGGPYMFQWNDDRCNMKNNFICKYSDEKPA
VPSREAEGEETELTTPVLPEETQEEDAKKTFKESREAALNLAYILIPSIPLLLLLVVTTVVCWVWIC
RKRQKTGAARP* (SEQ ID NO: 4)
>ENSG00000204381_ENST00000525866_ENSP00000434300_LAYN
MRPGTALQAVLLAVLLVGLRAATGRLLSGQPVCRGGTQRPCYKVIYFHDTSRRLNFEEAKEACR
RDGGQLVSIESEDEQKLIEKFIENLLPSDGDFWIGLRRREEKQSNSTACQDLYAWTDGSISQFRET
SSSF* (SEQ ID NO: 5)
>ENSG00000204381_ENST00000528924_ENSP00000486561_LAYN
MVTSGLGSGGVRRNKAIAQPARTFMLGLMAAYHNLEKPAVPSREAEGEETELTTPVLPEETQEE
DAKKTFKESREAALNLAYILIPSIPLLLLLVVTTVVCWVWICRK (SEQ ID NO: 6)
>ENSG00000204381_ENST00000530962_ENSP00000431627_LAYN
MYHQPSAPAGIGGPYMFQWNDDRCNMKNNFICKYSDEKPAVPSREAEGEETELTTPVLPEETQE
EDAKKTFKESREAALNLAYILIPSIPLLLLLVVTTVVCWVWICRK (SEQ ID NO: 7)
>ENSG00000204381_ENST00000533265_ENSP00000434972_LAYN
MRPGTALQAVLLAVLLVGLRAATGRLLSGQPVCRGGTQRPCYKVIYFHDTSRRLNFEEAKEACR
RDGGQLVSIESEDEQKLIEKFIENLLPSDGDFWIGLRRREEKQSNSTACQDLYAWTDGSISQFRN
WYVDEPSCGSEVCVVMYHQPSAPAGIGGPYMFQWNDDRCNMKNNFICKYSDEKPAVPSREAE
GEETELTTPVLPEETQEEDAKKTFKESREAALNLAYILIPSIPLLLLLVVTTVVCWVWICRKRQKT
GAARP* (SEQ ID NO: 8)
>ENSG00000204381_ENST00000533999_ENSP00000432434_LAYN
MYHQPSAPAGIGGPYMFQWNDDRCNMKNNFICKYSDEKPAVPSREAEGE (SEQ ID NO: 9)]),
CCR8 (SEQ ID Nos:10-11
[>ENSG00000179934_ENST00000326306_ENSP00000326432_CCR8
MDYTLDLSVTTVTDYYYPDIFSSPCDAELIQTNGKLLLAVFYCLLFVFSLLGNSLVILVLVVCKKL
RSITDVYLLNLALSDLLFVFSFPFQTYYLLDQWVFGTVMCKVVSGFYYIGFYSSMFFITLMSVDR
YLAVVHAVYALKVRTIRMGTTLCLAVWLTAIMATIPLLVFYQVASEDGVLQCYSFYNQQTLKW
KIFTNFKMNILGLLIPFTIFMFCYIKILHQLKRCQNHNKTKAIRLVLIVVIASLLFWVPFNVVLFLTS
LHSMHILDGCSISQQLTYATHVTEIISFTHCCVNPVIYAFVGEKFKKHLSEIFQKSCSQIFNYLGRQ
MPRESCEKSSSCQQHSSRSSSVDYIL* (SEQ ID NO: 10)
>ENSG00000179934_ENST00000414803_ENSP00000390104_CCR8
MDYTLDLSVTTVTDYYYPDIFSSPCDAELIQTNDLLSAGPVGVWDCNVQSGVWLLLHWLLQQH
VFHHPHECGQVPGCCPCRVCPKGEDDQDGHNAVPGSMANRHYGYHPIASVLPSGL* (SEQ ID
NO: 11)]),
IL21R (SEQ ID Nos: 12-14
[>ENSG00000103522_ENST00000337929_ENSP00000338010_IL21R
MPRGWAAPLLLLLLQGGWGCPDLVCYTDYLQTVICILEMWNLHPSTLTLTWQDQYEELKDEAT
SCSLHRSAHNATHATYTCHMDVFHFMADDIFSVNITDQSGNYSQECGSFLLAESIKPAPPFNVTV
TFSGQYNISWRSDYEDPAFYMLKGKLQYELQYRNRGDPWAVSPRRKLISVDSRSVSLLPLEFRK
DSSYELQVRAGPMPGSSYQGTWSEWSDPVIFQTQSEELKEGWNPHLLLLLLLVIVFIPAFWSLKT
HPLWRLWKKIWAVPSPERFFMPLYKGCSGDFKKWVGAPFTGSSLELGPWSPEVPSTLEVYSCHP
PRSPAKRLQLTELQEPAELVESDGVPKPSFWPTAQNSGGSAYSEERDRPYGLVSIDTVTVLDAEG
PCTWPCSCEDDGYPALDLDAGLEPSPGLEDPLLDAGTTVLSCGCVSAGSPGLGGPLGSLLDRLKP
PLADGEDWAGGLPWGGRSPGGVSESEAGSPLAGLDMDTFDSGFVGSDCSSPVECDFTSPGDEGP
PRSYLRQWVVIPPPLSSPGPQAS* (SEQ ID NO: 12)
>ENSG00000103522_ENST00000395754_ENSP00000379103_IL21R
MPRGWAAPLLLLLLQGGWGCPDLVCYTDYLQTVICILEMWNLHPSTLTLTWQDQYEELKDEAT
SCSLHRSAHNATHATYTCHMDVFHFMADDIFSVNITDQSGNYSQECGSFLLAESIKPAPPFNVTV
TFSGQYNISWRSDYEDPAFYMLKGKLQYELQYRNRGDPWAVSPRRKLISVDSRSVSLLPLEFRK
HPLWRLWKKIWAVPSPERFFMPLYKGCSGDFKKWVGAPFTGSSLELGPWSPEVPSTLEVYSCHP
PRSPAKRLQLTELQEPAELVESDGVPKPSFWPTAQNSGGSAYSEERDRPYGLVSIDTVTVLDAEG
PCTWPCSCEDDGYPALDLDAGLEPSPGLEDPLLDAGTTVLSCGCVSAGSPGLGGPLGSLLDRLKP
PLADGEDWAGGLPWGGRSPGGVSESEAGSPLAGLDMDTFDSGFVGSDCSSPVECDFTSPGDEGP
PRSYLRQWVVIPPPLSSPGPQAS* (SEQ ID NO: 13)
>ENSG00000103522_ENST00000564089_ENSP00000456707_IL21R
MPRGWAAPLLLLLLQGGWGCPDLVCYTDYLQTVICILEMWNLHPSTLTLTWQDQYEELKDEAT
SCSLHRSAHNATHATYTCHMDVFHFMADDIFSVNITDQSGNYSQECGSFLLAESIKPAPPFNVTV
TFSGQYNISWRSDYEDPAFYMLKGKLQYELQYRNRGDPWAVSPRRKLISVDSRSVSLLPLEFRK
DSSYELQVRAGPMPGSSYQGTWSEWSDPVIFQTQSEELKEGWNPHLLLLLLLVIVFIPAFWSLKT
HPLWRLWKKIWAVPSPERFFMPLYKGCSGDFKKWVGAPFTGSSLELGPWSPEVPSTLEVYSCHP
PRSPAKRLQLTELQEPAELVESDGVPKPSFWPTAQNSGGSAYSEERDRPYGLVSIDTVTVLDAEG
PCTWPCSCEDDGYPALDLDAGLEPSPGLEDPLLDAGTTVLSCGCVSAGSPGLGGPLGSLLDRLKP
PLADGEDWAGGLPWGGRSPGGVSESEAGSPLAGLDMDTFDSGFVGSDCSSPVECDFTSPGDEGP
PRSYLRQWVVIPPPLSSPGPQAS* (SEQ ID NO: 14)]).

Said cytokine is preferably selected from the group of consisting of: IL32 (SEQ ID Nos: 19-30), IL7 (SEQ ID Nos: 168-174), EB13 (SEQ ID NO: 175), SECTMI (SEQ ID Nos: 349-356), CSF1 (SEQ ID Nos: 585-592) and LTA (SEQ ID Nos: 657-658).

Said epigenetic factor is preferably selected from the group of consisting of: TDRD3 (SEQ ID NO: 712-718), KAT2B (SEQ ID NO:719), FOXA1 (SEQ ID Nos: 720-721) and RCBTB1 (SEQ ID Nos: 722-723).

Said kinase phosphatase is preferably selected from the group of consisting of: GSK3B (SEQ ID Nos: 724-725), SSH1 (SEQ ID NOS:111-112), CDK6 (SEQ ID Nos: 726-727), MINPP1 (SEQ ID Nos:181-183), PTPRJ (SEQ ID Nos: 395-400), CALM3 (SEQ ID Nos: 728-734) and PTP4A3 (SEQ ID Nos: 593-598).

Said transcription factor is preferably selected from the group of consisting of: VDR (SEQ ID NO:204), ZNF334 (SEQ ID Nos: 736-741), CREB3L2 (SEQ ID Nos: 565-567), ETV7 (SEQ ID NO:31 or 32), SOX4 (SEQ ID NO:735), TWIST1 (SEQ ID Nos: 743-745), TP73 (SEQ ID Nos: 746-756), FOXP3, NFE2L3 (SEQ ID NO:76), ARNTL2 (SEQ ID Nos: 757-764), BATF (SEQ ID Nos: 765-766), PTTG1 (SEQ ID Nos: 767-770), HIVEP3 (SEQ ID Nos: 771-772), FOXA1 (SEQ ID Nos: 720-721), ZBTB38 (SEQ ID NO:561), FOXM1 (SEQ ID Nos: 773-778), TADA3 (SEQ ID Nos: 779-782), NFAT5 (SEQ ID NO:160, 783-791, 742).

In a preferred embodiment, the marker is MAGEH1 (SEQ ID NO: 708 or 709)

[MAGEH1_Entrez:28986_ENSG00000187601_ENST00000342972_ENSP00000343706
ATGCCTCGGGGACGAAAGAGTCGGCGCCGCCGTAATGCGAGAGCCGCAGAAGAGAACCGC
AACAATCGCAAAATCCAGGCCTCAGAGGCCTCCGAGACCCCTATGGCCGCCTCTGTGGTAGC
GAGCACCCCCGAAGACGACCTGAGCGGCCCCGAGGAAGACCCGAGCACTCCAGAGGAGGC
CTCTACCACCCCTGAAGAAGCCTCGAGCACTGCCCAAGCACAAAAGCCTTCAGTGCCCCGGA
GCAATTTTCAGGGCACCAAGAAAAGTCTCCTGATGTCTATATTAGCGCTCATCTTCATCATG
GGCAACAGCGCCAAGGAAGCTCTGGTCTGGAAAGTGCTGGGGAAGTTAGGAATGCAGCCTG
GACGTCAGCACAGCATCTTTGGAGATCCGAAGAAGATCGTCACAGAAGAGTTTGTGCGCAG
AGGGTACCTGATTTATAAACCGGTGCCCCGTAGCAGTCCGGTGGAGTATGAGTTCTTCTGGG
GGCCCCGAGCACACGTGGAATCGAGCAAACTGAAAGTCATGCATTTTGTGGCAAGGGTTCG
TAACCGATGCTCTAAAGACTGGCCTTGTAATTATGACTGGGATTCGGACGATGATGCAGAGG
TTGAGGCTATCCTCAATTCAGGTGCTAGGGGTTATTCCGCCCCTTAA (SEQ ID NO: 708)
MPRGRKSRRRRNARAAEENRNNRKIQASEASETPMAASVVASTPEDDLSGPEEDPSTPEEASTTP
EEASSTAQAQKPSVPRSNFQGTKKSLLMSILALIFIMGNSAKEALVWKVLGKLGMQPGRQHSIFG
DPKKIVTEEFVRRGYLIYKPVPRSSPVEYEFFWGPRAHVESSKLKVMHFVARVRNRCSKDWPCN
YDWDSDDDAEVEAILNSGARGYSAP* (SEQ ID NO: 709)].

In the present invention, the tumor is preferably a solid or liquid tumor. Preferably, the solid tumor is selected from the group consisting of: non-small cell lung cancer, colorectal cancer, breast cancer, gastric cancer.

In a preferred embodiment of the invention, the tumor is a metastasis, preferably a bone, a brain or a liver metastasis.

Preferably, the metastasis derives from colon rectal cancer or non-small-cell lung cancer.

Another object of the invention is the above defined molecule for use in a method for in vivo depleting tumor-infiltrating regulatory T cells in a subject or for use in a method to enhance tumor immunity in a subject.

Another object of the invention is a pharmaceutical composition comprising the molecule as defined above and at least one pharmaceutically acceptable carrier.

A further object of the invention is a pharmaceutical composition comprising the molecule as above defined, for use in the prevention and/or treatment of tumor or for use in a method for in vivo depleting tumor-infiltrating regulatory T cell in a subject or for use in a method to enhance tumor immunity in a subject.

The pharmaceutical composition according to the invention may further comprise a therapeutic agent, preferably the therapeutic agent in an anti-tumoral agent.

Another object of the invention is an in vitro method for diagnosing and/or assessing the risk of developing and/or prognosing and/or for monitoring the progression and/or for monitoring the efficacy of a therapeutic treatment and/or for the screening of a therapeutic treatment of a tumour in a subject comprising the steps of:
a) detecting at least one of the marker as above defined in an isolated biological sample obtained from the subject and
b) comparing with respect to a proper control.

Another object of the invention is an in vitro or ex-vivo method for diagnosing and/or assessing the risk of developing and/or prognosing and/or for monitoring the progression and/or for monitoring the efficacy of a therapeutic treatment and/or for the screening of a therapeutic treatment of a tumour in a subject as above defined, wherein the marker to be detected is at least one of the marker selected from the group consisting of: LAYN, MAGEH1 and CCR8.

Preferably the above method is for prognosing of colorectal cancer or non-small cell lung cancer in a subject and comprises the steps of:
a) detecting at least one of the marker selected from the group consisting of: LAYN, MAGEH1 and CCR8 in an isolated biological sample obtained from the subject and
b) comparing with respect to a proper control,
wherein an amount of said at least one marker in the isolated biological sample obtained from the subject higher than the control amount indicates that the subject has a poor prognosis.

In the above method, preferably step a) comprises measuring the amount of the marker or of fragments thereof or of the polynucleotide coding for said protein (DNA or mRNA) or of fragments thereof in said isolated biological sample obtained from the subject and step b) comprises comparing the measured amount of step a) with a proper control amount.

Preferably, the in vitro method for monitoring the progression and/or for monitoring the efficacy of a therapeutic treatment of a tumour, as above defined, comprises the steps of:
a) measuring the alteration of the amount or the alteration of the activity of the above markers or of fragments thereof or of the polynucleotide coding for said protein or fragments thereof in said isolated biological sample obtained from the subject and
b) comparing the measured alteration of step a) with a proper control alteration.

Another object of the invention is a method for the treatment and/or prevention of tumor comprising administering to a subject the molecule as above defined.

A further object is a method for identifying a molecule acting as an anti-tumoral, comprising the steps of:
assaying candidate molecules for their binding specificity to the at least one marker as above defined;
selecting molecules having a specific binding activity to the at least one marker as above defined;
testing such specific binding molecules for their capacity of inhibiting proliferation and/or inducing an apoptotic response in a cell system,
preferably by selectively depleting tumor-infiltrating regulatory T cell, more preferably by inducing antibody-dependent cell-mediated cytotoxicity (ADCC).

Preferably, the biological sample is a fluid, a cell or a tissue sample, more preferably said sample is plasma or serum.

The term “biological sample” encompasses a clinical sample, and also includes tissue obtained by surgical resection, tissue obtained by biopsy, cells in culture, cell supernatants, cell lysates, tissue samples, organs, bone marrow, blood, plasma, serum, and the like.

A “sample” in the context of the present teachings refers to any biological sample that is isolated from a subject. A sample can include, without limitation an aliquot of body fluid, whole blood, serum, plasma, solid tissue samples such as tissue biopsies, or tissue cultures or cells derived therefrom and the progeny thereof, synovial fluid, lymphatic fluid, ascites fluid, and interstitial or extracellular fluid. The term “sample” also encompasses the fluid in spaces between cells, including gingival crevicular fluid, bone marrow, cerebrospinal fluid (CSF), saliva, mucous, sputum, semen, sweat, urine, or any other bodily fluids. “Blood sample” can refer to whole blood or any fraction thereof, including serum and plasma. Samples can be obtained from a subject by means including but not limited to venipuncture, excretion, ejaculation, massage, biopsy, needle aspirate, lavage, scraping, surgical incision, or intervention or other means known in the art. The definition also includes samples that have been manipulated in any way after their procurement, such as by treatment with reagents; washed; or enrichment for certain cell populations, such as cancer cells or samples in which regulatory T cells, are isolated and then analyzed. The definition also includes sample that have been enriched for particular types of molecules, e.g., nucleic acids, polypeptides, etc.

Another object of the invention is a kit for carrying out the above methods, comprising
means to measure the amount or the activity of the above markers or of fragments thereof and/or means to measure the amount of the polynucleotide coding for said protein or of fragments thereof and optionally,
control means.

Any combination of the above markers is comprised within the present invention.

Preferred combinations of markers are LAYN and MAGEH1; LAYN and CCR8; CCR8 and MAGEH1; LAYN, MAGEH1 and CCR8.

Preferably, the above polynucleotide is an RNAi inhibitor, preferably selected from the group consisting of: siRNA, miRNA, shRNA, stRNA, snRNA, and antisense nucleic acid, or a functional derivative thereof.

A comparative analysis of gene expression arrays from CD4+ T cells infiltrating NSCLC and CRC revealed Treg-specific expression of 328 markers as listed in Table IV Manipulation of Treg cells via these markers can therefore be used to enhance immunotherapy of cancer.

The expression “molecule able to modulate” and “modulator” are herein interchangeable.

By the term “modulator” it is meant a molecule that effects a change in the expression and/or function of at least one marker as above defined.

The change is relative to the normal or baseline level of expression and/or function in the absence of the modulator, but otherwise under similar conditions, and it may represent an increase (e.g. by using an inducer or activator) or a decrease (e.g. by using a suppressor or inhibitor) in the normal/baseline expression and/or function. In the context of the present invention, a “modulator” is a molecule which may suppress or inhibit the expression and/or function of at least one marker that is selectively deregulated in tumor-infiltrating regulatory T cell for use in the prevention and/or treatment of cancer.

By the term “suppressor or inhibitor” or a “molecule which (selectively) suppresses or inhibits” it is meant a molecule that effects a change in the expression and/or function of the target.

In the context of the present invention, a “modulator” is a molecule which may induce or activate the expression and/or function of at least one marker that is selectively deregulated in tumor-infiltrating regulatory T cell for use in the prevention and/or treatment of cancer.

The change is relative to the normal or baseline level of expression and/or function in the absence of the modulator, but otherwise under similar conditions, and it may represent an increase (e.g. by using an inducer or activator) or a decrease (e.g. by using a suppressor or inhibitor) in the normal/baseline expression and/or function.

The suppression or inhibition of the expression and/or function of the target may be assessed by any means known to the skilled in the art. The assessment of the expression level or of the presence of the target is preferably performed using classical molecular biology techniques such as (real time Polymerase Chain Reaction) qPCR, microarrays, bead arrays, RNAse protection analysis or Northern blot analysis or cloning and sequencing.

The assessment of target function is preferably performed by in vitro suppression assay, whole transcriptome analysis, mass spectrometry analysis to identify proteins interacting with the target.

In the context of the present invention, the target (or the marker) may be the gene, the mRNA, the cDNA, or the encoded protein thereof, including fragments, derivatives, variants, isoforms, etc. Preferably, the marker is characterized by its Accession numbers (i.e. NCBI Entrez ID; Ensembl Gene accession number (ENSG), Ensembl transcript accession number (ENST) and Ensembl protein accession number (ENSP), retrievable in the public database EnsEMBL (http://www.ensembl.orq) and/or amino acid and nucleotide sequences, herein disclosed.

In the context of the present invention, the term “treat” (or “treated”, “treatment”, etc.) when referred to CD4+ T cell, means e.g. the exposure of the cell to an exogenous modulator as above defined. The overexpression may be obtained e.g. by infecting the cells with a viral vector expressing the molecule of the invention. The inhibition of marker expression may e.g. by obtained by transfection with polynucleotide, as e.g. with siRNAs.

The term “treat” may also mean that the cells are manipulated in order to overexpress or silence the marker. The overexpression or the silencing may be obtained e.g. by genetically modifying the cells.

Control means can be used to compare the amount or the increase of amount of the marker defined to a proper control. The proper control may be obtained for example, with reference to known standard, either from a normal subject or from normal population, or from T cells different from tumour infiltrating regulatory T cells or regulatory T cells. The means to measure the amount of at least one marker as above defined are preferably at least one antibody, functional analogous or derivatives thereof. Said antibody, functional analogous or derivatives thereof are specific for said marker.

In the context of the present invention, the antibody is preferably selected from the group consisting of an intact immunoglobulin, a Fv, a scFv (single chain Fv fragment), a Fab, a F(ab′)2, an “antibody-like” domain, an “antibody-mimetic domain”, a single antibody domain (VH domain or VL domains), a multimeric antibody, recombinant or synthetic antigen-binding fragments, a peptide or a proteolytic fragment containing the epitope binding region. The terms “antibody” and “immunoglobulin” can be used interchangeably and are herein used in the broadest sense and encompass various antibodies and antibody mimetics structures, including but not limited to monoclonal antibodies, polyclonal antibodies, multispecific antibodies (e.g., bispecific antibodies), chimeric antibodies, nanobodies, antibody derivatives, antibody fragments, anticalins, DARPins, affibodies, affilins, affimers, affitins, alphabodies, avimers, fynomers, monobodies and other binding domains, so long as they exhibit the desired antigen-binding activity.

The term immunoglobulin also includes “conjugate” thereof. In the context of the present invention “conjugate” in relation to the antibody of the invention includes antibodies (or fragments thereof) conjugated with a substance (a compound, etc.) having a therapeutic activity, e.g. anti-tumor activity and/or cell-killing activity or a cytotoxic agents such as various A chain toxins, ribosomes inactivating proteins, and ribonucleases; bispecific antibodies designed to induce cellular mechanisms for killing tumors (see, for example, U.S. Pat. Nos. 4,676,980 and 4,954,617). The conjugate may be formed by previously preparing each of the aforementioned antibody molecule and the aforementioned substance having anti-tumor activity and/or cell-killing activity, separately, and then combining them (immunoconjugate) or by ligating a protein toxin used as such a substance having anti-tumor activity and/or cell-killing activity to an antibody gene on a gene according to a genetic recombination technique, so as to allow it to express as a single protein (a fusion protein) (immunotoxin).

An “antibody fragment” refers to a molecule other than an intact antibody that comprises a portion of an intact antibody that binds the antigen to which the intact antibody binds.

Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments. VH or VL Fvs are also called “Nanobodies”.

The term “antibody mimetics” refers to those organic compounds or binding domains that are not antibody derivatives but that can bind specifically an antigen like antibodies do.

They include anticalins, DARPins, affibodies, affilins, affimers, affitins, alphabodies, avimers, fynomers, monobodies and others.

The term “chimeric” antibody refers to an antibody in which a portion of the heavy and/or light chain is derived from a particular source or species, while the remainder of the heavy and/or light chain is derived from a different source or species.

The terms “full length antibody,” “intact antibody,” and “whole antibody” are used herein interchangeably to refer to an antibody having a structure substantially similar to a native antibody structure or having heavy chains that contain an Fc region as defined herein.

In a preferred embodiment, the kit of the invention comprises:
a solid phase adhered antibody specific for said compound;
detection means of the ligand specific-marker complex.

Alternatively, the reagents can be provided as a kit comprising reagents in a suspension or suspendable form, e.g. reagents bound to beads suitable for flow cytometry, preferably magnetic beads coated with antibody capture. The instructions may comprise instructions for conducting an antibody-based flow cytometry assay.

Detection means are preferably means able to detect and/or measure the amount of the described markers, e.g. means able to detect the complex antigen-antibody, as enzyme conjugated secondary antibodies, luminescent substrates, magnetic beads coated with antibody capture, customized dried antibody cocktails and/or columns with size filter cartridges and/or combined with specific antibody filter (SAF).

In an embodiment, the method further comprises selecting a therapeutic regimen based on the analysis. In an embodiment, the method further comprises determining a treatment course for the subject based on the analysis. Other means may be e.g. specific primers and probes for RT PCR. The kits according to the invention can further comprise customary auxiliaries, such as buffers, carriers, markers, etc. and/or instructions for use.

In the context of the present invention the term “detecting” may be intended also as “measuring the amount” or “measuring the alteration”. In the case of a method or a kit for assessing the risk and/or diagnosing and/or prognosing of a tumour, the proper control may be a sample taken from a healthy patient or from a patient affected by another disorder or pathology, and the proper control amount or activity may be the amount or activity of the same protein or polynucleotide measured in a sample taken from a healthy patient or from a patient affected by another disorder or pathology.

In the case of a method or a kit for monitoring the progression of a tumour, the progress of the cancer is monitored and the proper control may be a sample taken from the same subject at various times or from another patient, and the proper control amount or activity may by the amount or activity of the same protein or polynucleotide measured in a sample taken from the same subject at various times or from another patient.

In the case of a method or a kit for monitoring the efficacy of a therapeutic treatment, the proper control may by a sample taken from the same subject before initiation of the therapy or taken at various times during the course of the therapy and the proper control amount or activity may be the amount or activity of the same protein or polynucleotide measured in a sample taken from the same subject before initiation of the therapy or taken at various times during the course of the therapy.

In the case of a method or a kit for the screening of a therapeutic treatment, the proper control may be a sample taken from subjects without treatment and from subjects treated with a substance that is to be assayed or from subjects treated with a reference treatment and the proper control amount or activity may be the average of the amounts or activity of the same protein or polynucleotide measured in samples taken from subjects without treatment and from subjects treated with a substance that is to be assayed or from subjects treated with a reference treatment. In this case, if the amount or activity of MAGEH1 and/or LAYN and/or CCR8 or polynucleotides thereof in the isolated biological sample obtained from the subject is lower or equal than the control amount or activity, it may indicate that the tested substance is effective for the treatment of the tumour.

In the present invention, the expression “measuring the amount” can be intended as measuring the amount (or the activity) or concentration or level of the respective protein and/or mRNA thereof and/or DNA thereof, preferably semi-quantitative or quantitative.

Measurement of a protein can be performed directly or indirectly. Direct measurement refers to the amount or concentration measure of the marker, based on a signal obtained directly from the protein, and which is directly correlated with the number of protein molecules present in the sample. This signal—which can also be referred to as intensity signal—can be obtained, for example, by measuring an intensity value of a chemical or physical property of the marker. Indirect measurements include the measurement obtained from a secondary component (e.g., a different component from the gene expression product) and a biological measurement system (e.g. the measurement of cellular responses, ligands, “tags” or enzymatic reaction products).

The term “amount”, as used in the description refers but is not limited to the absolute or relative amount of proteins and/or mRNA thereof and/or DNA thereof, and any other value or parameter associated with the same or which may result from these. Such values or parameters comprise intensity values of the signal obtained from either physical or chemical properties of the protein, obtained by direct measurement, for example, intensity values in an immunoassay, mass spectroscopy or a nuclear magnetic resonance.

Additionally, these values or parameters include those obtained by indirect measurement, for example, any of the measurement systems described herein. Methods of measuring mRNA and DNA in samples are known in the art. To measure nucleic acid levels, the cells in a test sample can be lysed, and the levels of mRNA in the lysates or in RNA purified or semi-purified from lysates can be measured by any variety of methods familiar to those in the art. Such methods include hybridization assays using detectably labeled DNA or RNA probes (i.e., Northern blotting) or quantitative or semi-quantitative RT-PCR methodologies using appropriate oligonucleotide primers. Alternatively, quantitative or semi-quantitative in situ hybridization assays can be carried out using, for example, tissue sections, or unlysed cell suspensions, and detectably labeled (e.g., fluorescent, or enzyme-labeled) DNA or RNA probes. Additional methods for quantifying mRNA include RNA protection assay (RPA), cDNA and oligonucleotide microarrays, representation difference analysis (RDA), differential display, EST sequence analysis, and serial analysis of gene expression (SAGE).

If by comparing the measured amount or activity of the above markers or of the polynucleotide coding for said protein with the amount or activity obtained from a control sample, the amount or the activity of said marker in the sample isolated from the subject corresponds to a higher value, the subject may present cancer or go towards an aggravation of said disease.

If by comparing the measured amount or activity of the above markers or of the polynucleotide coding for said protein with the amount or the activity obtained from a control sample, the amount or the activity of said marker in the sample isolated from the subject corresponds to a similar or lower value, the subject may be not affected by cancer or go toward an amelioration of cancer, respectively.

Alternatively, the expression “detecting” or “measuring the amount” is intended as measuring the alteration of the molecule. Said alteration can reflect an increase or a decrease in the amount or activity of the molecules as above defined. An increase of the protein or of the activity of the marker or of the polynucleotide coding for said marker can be correlated to an aggravation of cancer. A decrease of the protein or of the activity of said marker or of the polynucleotide coding for said protein can be correlated to an amelioration of cancer or to recovery of the subject.

The expression “marker” is intended to include also the corresponding protein encoded from said marker orthologous or homologous genes, functional mutants, functional derivatives, functional fragments or analogues, isoforms, splice variants thereof.

When the expression “marker” is referred to genes, it is intended to include also the corresponding orthologous or homologous genes, functional mutants, functional derivatives, functional fragments or analogues, isoforms thereof.

As used herein “fragments” refers to polynucleotides having preferably a length of at least 1000 nucleotides, 1100 nucleotide, 1200 nucleotides, 1300 nucleotides, 1400 nucleotides, 1500 nucleotides.

As used herein “fragments” refers to polypeptides having preferably a length of at least amino acids, more preferably at least 15, at least 17 amino acids or at least 20 amino acids, even more preferably at least 25 amino acids or at least 37 or 40 amino acids, and more preferably of at least 50, or 100, or 150 or 200 or 250 or 300 or 350 or 400 or 450 or 500 amino acids.

The term “polynucleotide” also refers to modified polynucleotides.

As used herein, the term “vector” refers to an expression vector, and may be for example in the form of a plasmid, a viral particle, a phage, etc. Such vectors may include bacterial plasmids, phage DNA, baculovirus, yeast plasmids, vectors derived from combinations of plasmids and phage DNA, viral DNA such as vaccinia, adenovirus, lentivirus, fowl pox virus, and pseudorabies. Large numbers of suitable vectors are known to those of skill in the art and are commercially available.

The polynucleotide sequence, preferably the DNA sequence in the vector is operatively linked to an appropriate expression control sequence(s) (promoter) to direct mRNA synthesis. As representative examples of such promoters, one can mention prokaryotic or eukaryotic promoters such as CMV immediate early, HSV thymidine kinase, early and late SV40, LTRs from retrovirus, and mouse metallothionein-1. The expression vector may also contain a ribosome binding site for translation initiation and a transcription vector.

The vector may also include appropriate sequences for amplifying expression. In addition, the vectors preferably contain one or more selectable marker genes to provide a phenotypic trait for selection of transformed host cells such as dihydro folate reductase or neomycin resistance for eukaryotic cell culture, or such as tetracycline or ampicillin resistance in E. coli.

As used herein, the term “host cell genetically engineered” relates to host cells which have been transduced, transformed or transfected with the polynucleotide or with the vector described previously. As representative examples of appropriate host cells, one can cite bacterial cells, such as E. coli, Streptomyces, Salmonella typhimurium, fungal cells such as yeast, insect cells such as Sf9, animal cells such as CHO or COS, plant cells, etc. The selection of an appropriate host is deemed to be within the scope of those skilled in the art from the teachings herein. Preferably, said host cell is an animal cell, and most preferably a human cell.

The introduction of the polynucleotide or of the vector described previously into the host cell can be effected by method well known from one of skill in the art such as calcium phosphate transfection, DEAE-Dextran mediated transfection, electroporation, lipofection, microinjection, viral infection, thermal shock, transformation after chemical permeabilisation of the membrane or cell fusion.

The polynucleotide may be a vector such as for example a viral vector.

The polynucleotides as above defined can be introduced into the body of the subject to be treated as a nucleic acid within a vector which replicates into the host cells and produces the polynucleotides or the proteins.

Suitable administration routes of the pharmaceutical composition of the invention include, but are not limited to, oral, rectal, transmucosal, intestinal, enteral, topical, suppository, through inhalation, intrathecal, intraventricular, intraperitoneal, intranasal, intraocular and parenteral (e.g., intravenous, intramuscular, intramedullary, and subcutaneous). An additional suitable administration route includes chemoembolization. Other suitable administration methods include injection, viral transfer, use of liposomes, e.g. cationic liposomes, oral intake and/or dermal application.

In certain embodiments, a pharmaceutical composition of the present invention is administered in the form of a dosage unit (e.g., tablet, capsule, bolus, etc.).

For pharmaceutical applications, the composition may be in the form of a solution, e.g. an injectable solution, emulsion, suspension or the like. The carrier may be any suitable pharmaceutical carrier. Preferably, a carrier is used which is capable of increasing the efficacy of the RNA molecules to enter the target cells. Suitable examples of such carriers are liposomes.

The modulator as above defined is administered in a pharmaceutically effective dosage, which in the case of polynucleotides may be in the range of 0.001 pg/kg body weight to 10 mg/kg body weight depending on the route of administration and the type or severity of the disease.

The term “pharmaceutical composition” refers to a preparation which is in such form as to permit the biological activity of an active ingredient contained therein to be effective, and which contains no additional components which are unacceptably toxic to a subject to which the formulation would be administered. In the present invention the term “effective amount” shall mean an amount which achieves a desired effect or therapeutic effect as such effect is understood by those of ordinary skill in the art. In the present invention, the antibody may be administered simultaneously or sequentially with another therapeutic treatment, that may be a chemotherapy or radiotherapy. The invention provides formulations comprising a therapeutically effective amount of an antibody as disclosed herein, a buffer maintaining the pH in the range from about 4.5 to about 8.5, and, optionally, a surfactant. The formulations are typically for an antibody as disclosed herein, recombinant or synthetic antigen-binding fragments thereof of the invention as active principle concentration from about 0.1 mg/ml to about 100 mg/ml. In certain embodiments, the antibody, recombinant or synthetic antigen-binding fragments thereof concentration is from about 0.1 mg/ml to 1 mg/ml; preferably from 1 mg/ml to 10 mg/ml, preferably from 10 to 100 mg/ml.

Therapeutic formulations of the antibody/antibodies can be prepared by mixing the antibody having the desired degree of purity with optional physiologically acceptable carriers, excipients or stabilizers (Remington's Pharmaceutical Sciences 16th edition, Osol, A. Ed., 1980), in the form of lyophilized formulations or aqueous solutions.

Pharmaceutical compositions containing the antibody of the present invention may be manufactured by processes well known in the art, e.g., using a variety of well-known mixing, dissolving, granulating, levigating, emulsifying, encapsulating, entrapping or lyophilizing processes. Proper formulation is dependent upon the route of administration chosen. Parenteral routes are preferred in many aspects of the invention.

For injection, including, without limitation, intravenous, intramusclular and subcutaneous injection, the compounds of the invention may be formulated in aqueous solutions, preferably in physiologically compatible buffers such as physiological saline buffer or polar solvents including, without limitation, a pyrrolidone or dimethylsulfoxide.

Formulations for injection may be presented in unit dosage form, e.g. in ampoules or in multi-dose containers. Useful compositions include, without limitation, suspensions, solutions or emulsions in oily or aqueous vehicles, and may contain adjuncts such as suspending, stabilizing and/or dispersing agents. Pharmaceutical compositions for parenteral administration include aqueous solutions of a water soluble form, such as, without limitation, a salt of the active compound. Additionally, suspensions of the active compounds may be prepared in a lipophilic vehicle. Suitable lipophilic vehicles include fatty oils such as sesame oil, synthetic fatty acid esters such as ethyl oleate and triglycerides, or materials such as liposomes. Aqueous injection suspensions may contain substances that increase the viscosity of the suspension, such as sodium carboxymethyl cellulose, sorbitol, or dextran. Optionally, the suspension may also contain suitable stabilizers and/or agents that increase the solubility of the compounds to allow for the preparation of highly concentrated solutions. Alternatively, the active ingredient may be in powder form for constitution with a suitable vehicle, e.g., sterile, pyrogen-free water, before use. For administration by inhalation, the antibody of the present invention can conveniently be delivered in the form of an aerosol spray using a pressurized pack or a nebulizer and a suitable propellant. The antibody may also be formulated in rectal compositions such as suppositories or retention enemas, using, e.g., conventional suppository bases such as cocoa butter or other glycerides. In addition to the formulations described previously, the antibody may also be formulated as depot preparations. Such long acting formulations may be administered by implantation (for example, subcutaneously or intramuscularly) or by intramuscular injection. The compounds of this invention may be formulated for this route of administration with suitable polymeric or hydrophobic materials (for instance, in an emulsion with a pharmacologically acceptable oil), with ion exchange resins, or as a sparingly soluble derivative such as, without limitation, a sparingly soluble salt. Additionally, the antibody may be delivered using a sustained-release system, such as semi-permeable matrices of solid hydrophobic polymers containing the therapeutic agent. Other delivery systems such as liposomes and emulsions can also be used.

A therapeutically effective amount refers to an amount of compound effective to prevent, alleviate or ameliorate cancer or cancer recurrence symptoms. Determination of a therapeutically effective amount is well within the capability of those skilled in the art, especially in light of the disclosure herein. For any antibody used in the invention, the therapeutically effective amount can be estimated initially from in vitro assays. Then, the dosage can be formulated for use in animal models so as to achieve a circulating concentration range that includes the effective dosage. Such information can then be used to more accurately determine dosages useful in patients. The amount of the composition that is administered will depend upon the parent molecule included therein.

Generally, the amount used in the treatment methods is that amount which effectively achieves the desired therapeutic result in mammals. Naturally, the dosages of the various compounds can vary somewhat depending upon the compound, rate of in vivo hydrolysis, etc. In addition, the dosage, of course, can vary depending upon the dosage form and route of administration. The range set forth above is illustrative and those skilled in the art will determine the optimal dosing of the compound selected based on clinical experience and the treatment indication. Moreover, the exact formulation, route of administration and dosage can be selected by the individual physician in view of the patient's condition and of the most effective route of administration (e.g., intravenous, subcutaneous, intradermal). Additionally, toxicity and therapeutic efficacy of the antibody and other therapeutic agent described herein can be determined by standard pharmaceutical procedures in cell cultures or experimental animals using methods well-known in the art.

It is contemplated that the treatment will be given for one or more cycles until the desired clinical and biological result is obtained. The exact amount, frequency and period of administration of the compound of the present invention will vary, of course, depending upon the sex, age and medical condition of the patient as well as the severity and type of the disease as determined by the attending clinician.

The modulator of the present invention may comprise a single type of modulator or a plurality of different modulators.

The function of a regulatory T-cell may be inhibited by inhibiting markers activity and/or expression or by decreasing the number of cells positive for such markers in a T-cell population (for example by binding at least one of the above marker and inducing antibody-dependent cell-mediated cytotoxicity (ADCC)). Inhibiting the function of regulatory T-cells in an organism may be used to enhance the immune T-cell response in those circumstances where such a response is desirable, such as in a patient suffering from cancer.

When treating a cancer patient with an inhibitory agent that binds to marker protein or mRNA, one may optionally co-administer an anti-tumor vaccine or therapy. Such vaccines may be directed to isolated antigens or to groups of antigens or to whole tumor cells. It may be desirable to administer the inhibitory agent with chemotherapeutic agents or together with radiotherapy.

Treatment with multiple agents need not be done using a mixture of agents but may be done using separate pharmaceutical preparations. The preparations need not be delivered at the same exact time, but may be coordinated to be delivered to a patient during the same period of treatment, i.e. within a week or a month or each other.

Thus a composition comprising two active ingredients may be constituted in the body of the patient. Any suitable anti-tumor treatment can be coordinated with the treatments of the present invention targeted to the markers. Similarly, if treating patients with infections, other anti-infection agents can be coordinated with the treatment of the present invention targeted to the markers. Such agents may be small molecule drugs, vaccines, antibodies, etc.

The number of marker+ cells in a T-cell population can be modified by using an antibody or other agent that selectively binds to the marker. marker+ cells represent an enriched population of regulatory T-cells that can be introduced back into the original source of the T-cells or into another compatible host to enhance regulatory T-cell function. Alternatively, the marker-cells represent a population of T-cells deficient in regulatory T-cell activity that can be reintroduced into the original source of the T-cells or another compatible host to inhibit or reduce regulatory T-cell function while retaining general T-cell activity.

Any desired means for either increasing or decreasing (modulating) marker activity can be used in the methods of the invention. This includes directly modulating the function of marker protein, modulating marker signal transduction, and modulating expression of marker in T-cells by modulating either transcription or translation or both. Those means which selectively modulate marker activity are preferred over nonselective modulators.

Also, those inhibitory means which create a transient marker deficiency in a population of T-cells which then return to normal levels of marker activity may be preferred for treating a temporary T-cell deficiency. The transiently deficient T-cells may be used to reconstitute a diminished T-cell population with T-cells that will be genetically normal with respect to the marker. Modulation of marker activity can be performed on cells in vitro or in whole animals, in vivo. Cells which are treated in vitro can be administered to a patient, either the original source of the cells or an unrelated individual.

To inhibit the function of the marker (antagonist), marker antibodies or small molecule inhibitors can be used. Antibodies or antibody fragments that are useful for this purpose will be those that can bind to the marker and block its ability to function. Such antibodies may be polyclonal antibodies, monoclonal antibodies, chimeric antibodies, humanized antibodies, single-chain antibodies, soluble MHC class II molecules, antibody fragments, etc.

Antibodies generated against marker polypeptides can be obtained by direct injection of the marker polypeptides into an animal or by administering marker polypeptides to an animal, preferably a nonhuman. The antibody so obtained will then bind the marker polypeptides itself. In this manner, even a sequence encoding only a fragment of the marker polypeptide can be used to generate antibodies binding the whole native marker polypeptide.

For preparation of monoclonal antibodies, any technique which provides antibodies produced by continuous cell line cultures can be used. Examples include the hybridoma technique (Kohler and Milstein, 1975, Nature, 256: 495-497), the trioma technique, the human B-cell hybridoma technique (Kozbor et al., 1983, Immunology Today 4: 72), and the EBV-hybridoma technique to produce human monoclonal antibodies (Cole, et al; 1985, in Monoclonal Antibodies and Cancer Therapy, Alan R. Liss, Inc., pp. 77-96). Techniques described for the production of single chain antibodies (U.S. Pat. No. 4,946,778) can be readily used to produce single chain antibodies to marker polypeptides. Also, transgenic mice may be used to express humanized antibodies to immunogenic marker polypeptides. To enhance or activate the function of the marker, any agent which increases the level of the marker or the activity of existing marker in the T-cell may be used. Such agents may be identified using the screening assays described below. Expression vectors encoding the marker can also be administered to increase the gene dosage. The expression vectors can be plasmid vectors or viral vectors, as are known in the art. Any vector can be chosen by the skilled in the art for particularly desirable properties. In the context of the present invention, the term “polynucleotide” includes DNA molecules (e.g., cDNA or genomic DNA) and RNA molecules (e.g., mRNA, siRNA, shRNA) and analogues of the DNA or RNA generated using nucleotide analogues. The polynucleotide may be single-stranded or double-stranded. The polynucleotide may be synthesized using oligonucleotide analogues or derivatives (e.g., inosine or phosphorothioate nucleotides).

The RNAi inhibitors as above defined are preferably capable of hybridizing to all or part of specific target sequence. Therefore, RNAi inhibitors may be fully or partly complementary to all of or part of the target sequence

The RNAi inhibitors may hybridize to the specified target sequence under conditions of medium to high stringency.

An RNAi inhibitors may be defined with reference to a specific sequence identity to the reverse complement of the sequence to which it is intended to target. The antisense sequences will typically have at least about 75%, preferably at least about 80%, at least about 85%, at least about 90%, at least about 95% or at least about 99% sequence identity with the reverse complements of their target sequences.

The term polynucleotide and polypeptide also includes derivatives and functional fragments thereof.

In the context of the present invention, the at least one gene or marker as above defined is preferably characterized by at least one of the sequence identified by its Ensembl Gene ID or NCBI Accession Numbers, as disclosed in Tables VIII or VI, or by at least one of the SEQ ID No. 1-709.

The term gene herein also includes corresponding orthologous or homologous genes, isoforms, variants, allelic variants, functional derivatives, functional fragments thereof.

The expression “protein” is intended to include also the corresponding protein encoded from a corresponding orthologous or homologous genes, functional mutants, functional derivatives, functional fragments or analogues, isoforms thereof.

The term “analogue” as used herein referring to a protein means a modified peptide wherein one or more amino acid residues of the peptide have been substituted by other amino acid residues and/or wherein one or more amino acid residues have been deleted from the peptide and/or wherein one or more amino acid residues have been deleted from the peptide and or wherein one or more amino acid residues have been added to the peptide. Such addition or deletion of amino acid residues can take place at the N-terminal of the peptide and/or at the C-terminal of the peptide.

A “derivative” may be a nucleic acid molecule, as a DNA molecule, coding the polynucleotide as above defined, or a nucleic acid molecule comprising the polynucleotide as above defined, or a polynucleotide of complementary sequence. In the context of the present invention the term “derivatives” also refers to longer or shorter polynucleotides and/or polypeptides having e.g. a percentage of identity of at least 41%, 50%, 60%, 65%, 70% or 75%, more preferably of at least 85%, as an example of at least 90%, and even more preferably of at least 95% or 100% with the sequences herein mentioned or with their complementary sequence or with their DNA or RNA corresponding sequence. The term “derivatives” and the term “polynucleotide” also include modified synthetic oligonucleotides. The modified synthetic oligonucleotide are preferably LNA (Locked Nucleic Acid), phosphoro-thiolated oligos or methylated oligos, morpholinos, 2′-O-methyl, 2′-O-methoxyethyl oligonucleotides and cholesterol-conjugated 2′-O-methyl modified oligonucleotides (antagomirs).

The term “derivative” may also include nucleotide analogues, i.e. a naturally occurring ribonucleotide or deoxyribonucleotide substituted by a non-naturally occurring nucleotide.

The term “derivatives” also includes nucleic acids or polypeptides that may be generated by mutating one or more nucleotide or amino acid in their sequences, equivalents or precursor sequences. The term “derivatives” also includes at least one functional fragment of the polynucleotide.

In the context of the present invention “functional” is intended for example as “maintaining their activity”.

In the context of the present invention, the vector as above defined is preferably selected from the group consisting of: plasmids, viral vectors and phages, more preferably the viral vector is a lentiviral vector.

In the context of the present invention, the host cell as above defined is preferably selected from the group consisting of: bacterial cells, fungal cells, insect cells, animal cells, plant cells, preferably being an animal cell.

Compositions comprising a mixture of antibodies which specifically bind to the marker(s); and an anti-cancer vaccine can be made in vitro. Preferably the composition is made under conditions which render it suitable for use as a pharmaceutical composition.

Pharmaceutical compositions may be sterile and pyrogen-free. The components of the composition can also be administered separately to a patient within a period of time such that they are both within the patient's body at the same time. Such a time-separated administration leads to formation of the mixture of antibodies and vaccine within the patient's body. If the antibody and vaccine are to be administered in a time-separated fashion, they may be supplied together in a kit. Within the kit the components may be separately packaged or contained. Other components such as excipients, carriers, other immune modulators or adjuvants, instructions for administration of the antibody and the vaccine, and injection devices can be supplied in the kit as well. Instructions can be in a written, video, or audio form, can be contained on paper, an electronic medium, or even as a reference to another source, such as a website or reference manual.

Anti-marker antibodies of the invention can be used to increase the magnitude of anti-cancer response of the cancer patient to the anti-cancer vaccine or anti-cancer therapy.

It can also be used to increase the number of responders in a population of cancer patients. Thus the antibodies can be used to overcome immune suppression found in patients refractory to anti-cancer vaccines or treatment. The anti-cancer vaccines can be any that are known in the art, including, but not limited to whole tumor cell vaccines, isolated tumor antigens or polypeptides comprising one or more epitopes of tumor antigens.

Expression of marker in T-cells can be modulated at the transcriptional or translational level. Agents which are capable of such modulation can be identified using the screening assays described below.

Translation of marker mRNA can be inhibited by using ribozymes, antisense molecules, small interference RNA (siRNA; See Elbashir, S. M. et al., “Duplexes of 21-nucleotide RNAs mediate RNA interference in cultured mammalian cells”, Nature 411: 494-498 (2001)) or small molecule inhibitors of this process which target marker mRNA.

Antisense technology can be used to control gene expression through triple-helix formation or antisense DNA or RNA, both of which methods are based on binding of a polynucleotide to DNA or RNA. For example, the 5′coding portion of the polynucleotide sequence, which codes for the mature polypeptides of the present invention, is used to design an antisense RNA oligonucleotide of from about 10 to 40 base pairs in length. A DNA oligonucleotide is designed to be complementary to a region of the gene involved in transcription (triple helix—see Lee et al., Nucl. Acids Res., 6: 3073 (1979); Cooney et al, Science, 241: 456 (1988); and Dervan et al., Science, 251: 1360 (1991)), thereby preventing transcription and the production of the marker. The antisense RNA oligonucleotide hybridizes to the mRNA in vivo and blocks translation of the mRNA molecule into the marker polypeptide (Antisense—Okano, J. Neurochem., 56: 560 (1991); Oligodeoxynucleotides as Antisense Inhibitors of Gene Expression, CRC Press, Boca Raton, Fla. (1988)). The oligonucleotides described above can also be delivered to cells by antisense expression constructs such that the antisense RNA or DNA may be expressed in vivo to inhibit production of the marker. Such constructs are well known in the art. Antisense constructs, antisense oligonucleotides, RNA interference constructs or siRNA duplex RNA molecules can be used to interfere with expression of the marker.

Typically, at least 15, 17, 19, or 21 nucleotides of the complement of marker mRNA sequence are sufficient for an antisense molecule. Typically at least 19, 21, 22, or 23 nucleotides of marker are sufficient for an RNA interference molecule. Preferably an RNA interference molecule will have a 2 nucleotide 3′ overhang. If the RNA interference molecule is expressed in a cell from a construct, for example from a hairpin molecule or from an inverted repeat of the desired marker sequence, then the endogenous cellular machinery will create the overhangs. siRNA molecules can be prepared by chemical synthesis, in vitro transcription, or digestion of long dsRNA by Rnase III or Dicer. These can be introduced into cells by transfection, electroporation, or other methods known in the art. (See Hannon, G J, 2002, RNA Interference, Nature 418:244-251; Bernstein E et al., 2002, The rest is silence. RNA 7:1509-1521; Hutvagner G et aL9 RNAi: Nature harbors a double-strand. Curr. Opin. Genetics & Development 12: 225-232, 2002, A system for stable expression of short interfering RNAs in mammalian cells. Science 296: 550-553; Lee N S, Dohjima T, Bauer G, Li H, Li M-J, Ehsani A, Salvaterra P, and Rossi J. (2002). Expression of small interfering RNAs targeted against HIV-1 rev transcripts in human cells. Nature Biotechnol. 20: 500-505; Miyagishi M, and Taira K. (2002). U6-promoter-driven siRNAs with four uridine 3′ overhangs efficiently suppress targeted gene expression in mammalian cells. Nature Biotechnol. 20: 497-500; Paddison P J, Caudy A A, Bernstein E, Hannon G J, and Conklin D S. (2002). Short hairpin RNAs (shRNAs) induce sequence-specific silencing in mammalian cells. Genes & Dev. 16: 948-958; Paul C P, Good P D, Winer I, and Engelke D R. (2002). Effective expression of small interfering RNA in human cells. Nature Biotechnol. 20: 505-508; Sui G, Soohoo C, Affar E-B, Gay F, Shi Y, Forester W C, and Shi Y. (2002). A DNA vector-based RNAi technology to suppress gene expression in mammalian cells. Proc. Natl. Acad. Sci. USA 99 (6): 5515-5520; Yu J-Y, DeRuiter S L, and Turner D L. (2002). RNA interference by expression of short-interfering RNAs and hairpin RNAs in mammalian cells. Proc. Natl. Acad. Sci. USA 99 (9): 6047-6052).

In addition to known modulators, additional modulators of markers activity that are useful in the methods of the invention can be identified using two-hybrid screens, conventional biochemical approaches, and cell-based screening techniques, such as screening candidate molecules for an ability to bind to marker or screening for compounds which inhibit marker activity in cell culture.

This provides a simple in vitro assay system to screen for marker activity modulators. The method may identify agents that directly interact with and modulate the marker, as well as agents that indirectly modulate marker activity by affecting a step in the marker signal transduction pathway.

Cell-based assays employing cells which express the marker can employ cells which are isolated from mammals and which naturally express the marker. Alternatively, cells which have been genetically engineered to express the marker can be used. Preferably the genetically engineered cells are T-cells.

Agents which modulate the marker activity by modulating the markergene expression can be identified in cell based screening assays by measuring amounts of the marker protein in the cells in the presence and absence of candidate agents. The marker protein can be detected and measured, for example, by flow cytometry using anti-marker specific monoclonal antibodies. Marker mRNA can also be detected and measured using techniques known in the art, including but not limited to Northern blot, RT-PCR, and array hybridization.

In accordance with the teachings of the invention, marker inhibitors may be administered to an organism to increase the number of T-cells in the organism. This method may be useful for treating organisms suffering from conditions resulting in a low T-cell population.

Such conditions include disorders involving unwanted cellular invasion or growth, such as tumor growth or cancer. Marker inhibitors may also be useful when administered in combination with conventional therapeutics to treat T-cell proliferation sensitive disorders.

For instance, a tumor, which is a T-cell proliferation sensitive disorder, is conventionally treated with a chemotherapeutic agent which functions by killing rapidly dividing cells. The marker inhibitors of the invention when administered in conjunction with a chemotherapeutic agent enhance the tumoricidal effect of the chemotherapeutic agent by stimulating T-cell proliferation to enhance the immunological rejection of the tumor cells.

In accordance with the teachings of the invention, marker activators (agonists) or expression enhancers may be administered to an organism to decrease the number of T-cells, in particular tumor-infiltrating regulatory T cells, in the organism and thereby decrease deleterious T-cell activity. The methods of the invention may be applied to any organism which contains T-cells that express the marker. This includes, but is not limited to, any mammal and particularly includes humans and mice.

When methods of the invention are carried out in vivo, the effective amount of the marker modulator used will vary with the particular modulator being used, the particular condition being treated, the age and physical condition of the subject being treated, the severity of the condition, the duration of the treatment, the nature of the concurrent therapy (if any), the specific route of administration and similar factors within the knowledge and expertise of the health practitioner. For example, an effective amount can depend upon the degree to which an individual has abnormally depressed levels of T cells.

When administered, the pharmaceutical preparations of the invention are applied in pharmaceutically-acceptable amounts and in pharmaceutically-acceptably compositions.

Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts. Marker modulators may be combined, optionally, with a pharmaceutically-acceptable carrier. The term “pharmaceutically-acceptable carrier” as used herein means one or more compatible solid or liquid filler, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction which would substantially impair the desired pharmaceutical efficacy.

The pharmaceutical compositions may contain suitable buffering agents, including: acetic acid in a salt; citric acid in a salt; boric acid in a salt; and phosphoric acid in a salt. The pharmaceutical compositions also may contain, optionally, suitable preservatives, such as: benzalkonium chloride; chlorobutanol; parabens and thimerosal.

Compositions suitable for parenteral administration conveniently comprise a sterile aqueous preparation of the anti-inflammatory agent, which is preferably isotonic with the blood of the recipient. This aqueous preparation may be formulated according to known methods using suitable dispersing or wetting agents and suspending agents.

The sterile injectable preparation also may be a sterile injectable solution or suspension in a non-toxic parenterally-acceptable diluent or solvent, for example, as a solution in butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables.

Carrier formulation suitable for oral, subcutaneous, intravenous, intramuscular, etc. administrations can be found in Remington's Pharmaceutical Sciences, Mack Publishing Co., Easton, Pa.

A variety of administration routes are available. The particular mode selected will depend, of course, upon the particular drug selected, the severity of the condition being treated and the dosage required for therapeutic efficacy. The methods of the invention, generally speaking, may be practiced using any mode of administration that is medically acceptable, meaning any mode that produces effective levels of the active compounds without causing clinically unacceptable adverse effects. Such modes of administration include oral, rectal, topical, nasal, interdermal, or parenteral routes.

The term “parenteral” includes subcutaneous, intravenous, intramuscular, or infusion.

Intravenous or intramuscular routes are not particularly suitable for long-term therapy and prophylaxis. They could, however, be preferred in emergency situations. Oral administration will be preferred because of the convenience to the patient as well as the dosing schedule. The pharmaceutical compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active agent into association with a liquid carrier, a finely divided solid carrier, or both, and then, if necessary, shaping the product. Compositions suitable for oral administration may be presented as discrete units, such as capsules, tablets, lozenges, each containing a predetermined amount of the active agent. Other compositions include suspensions in aqueous liquids or non-aqueous liquids such as a syrup, elixir or an emulsion.

Other delivery systems can include time-release, delayed release or sustained release delivery systems. Such systems can avoid repeated administrations of the active agent, increasing convenience to the subject and the physician. Many types of release delivery systems are available and known to those of ordinary skill in the art. They include polymer base systems such as poly (lactide-glycolide), copolyoxalates, polycaprolactones, polyesteramides, polyorthoesters, polyhydroxybutyric acid, and polyanhydrides.

Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which the anti-inflammatory agent is contained in a form within a matrix such as those described in U.S. Pat. Nos. 4,452,775, 4,667,014, 4,748,034 and 5,239,660 and (b) diffusional systems in which an active component permeates at a controlled rate from a polymer such as described in U.S. Pat. Nos. 3,832,253, and 3,854,480. In addition, pump-based hardware delivery systems can be used, some of which are adapted for implantation.

Use of a long-term sustained release implant may be particularly suitable for treatment of chronic conditions. Long-term release, are used herein, means that the implant is constructed and arranged to deliver therapeutic levels of the active ingredient for at least 30 days, and preferably 60 days. Long-term sustained release implants are well-known to those of ordinary skill in the art and include some of the release systems described above. While the invention has been described with respect to specific examples including presently preferred modes of carrying out the invention, those skilled in the art will appreciate that there are numerous variations and permutations of the above described systems and techniques that fall within the spirit and scope of the invention as set forth in the appended claims.

The invention will be illustrated by means of non-limiting examples in reference to the following figures.

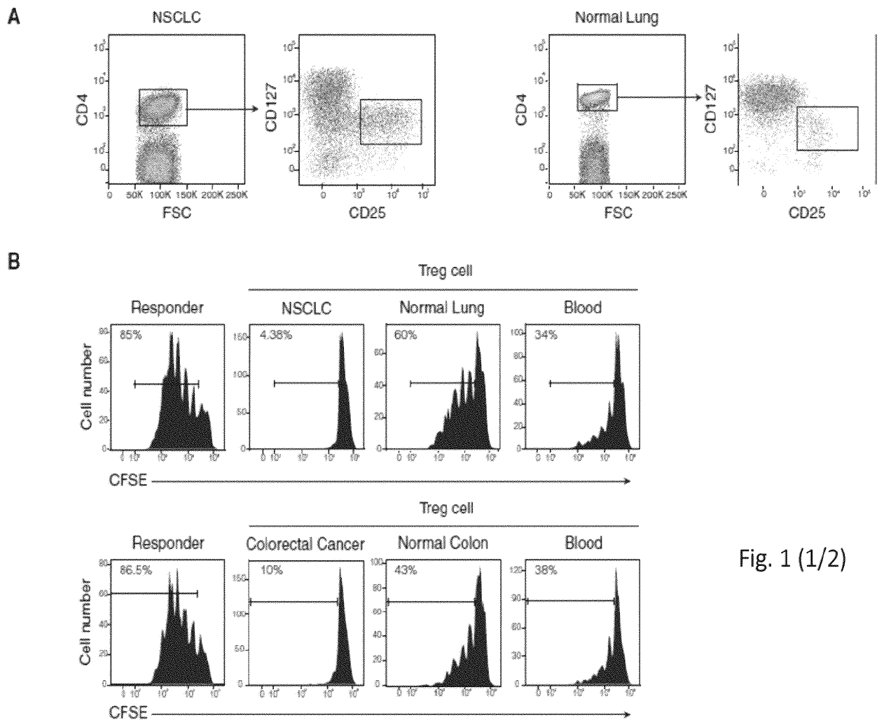

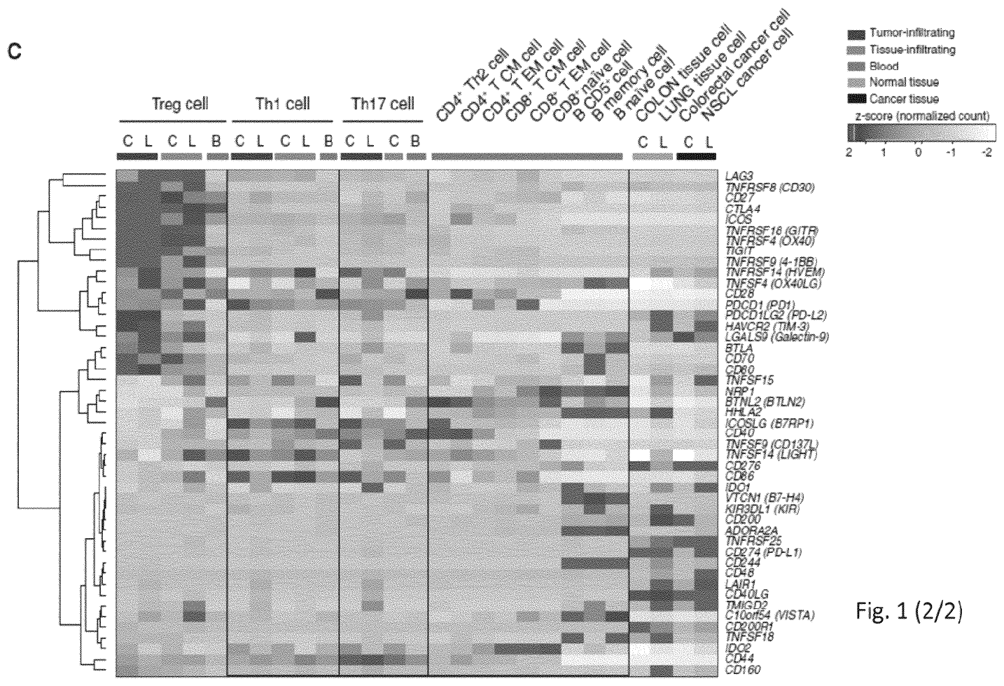

FIG. 1. Purification, functional characterization and expression of immune checkpoints in tumor infiltrating cells.

(A) Representation of the sorting strategy of Treg cells infiltrating tumor or normal tissue.

(B) Representative flow cytometry plots showing suppressive activity of Treg cells isolated from tumor (NSCLC or CRC), normal lung and blood of the same patient. 4×10⁵ carboxyfluorescein diacetate succinimidyl ester (CFSE)-labeled CD4+ naïve T cells from healthy donors were cocultured with an equal number of Treg cells for 4 days with a CD3-specific mAb and CD1c+CD11c+ dendritic cells. Percentage of proliferating cells are indicated. Data are representative of three independent experiments.

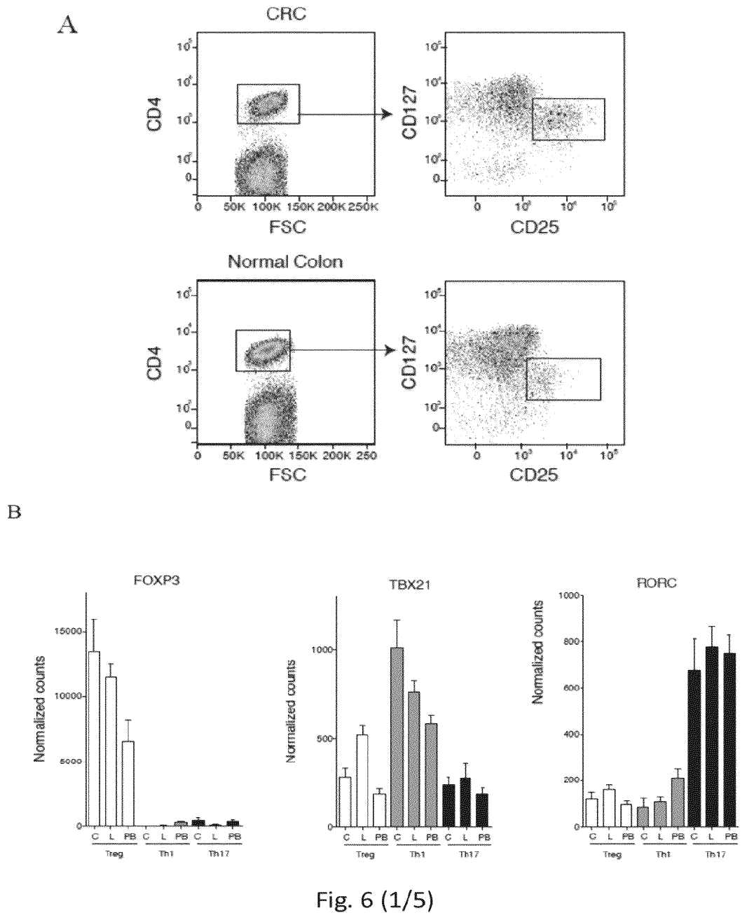

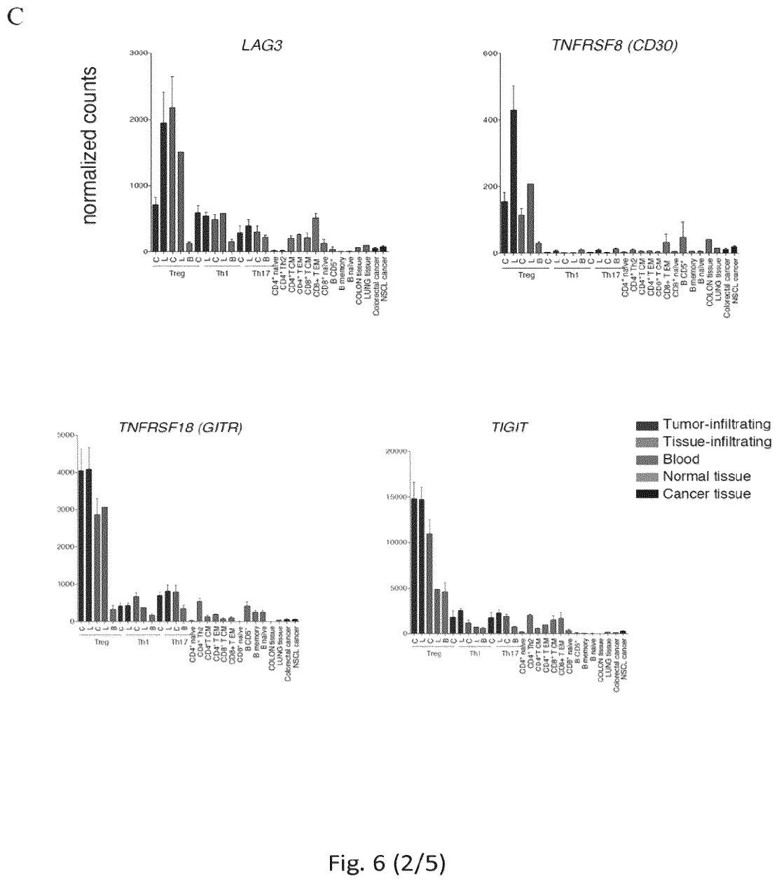

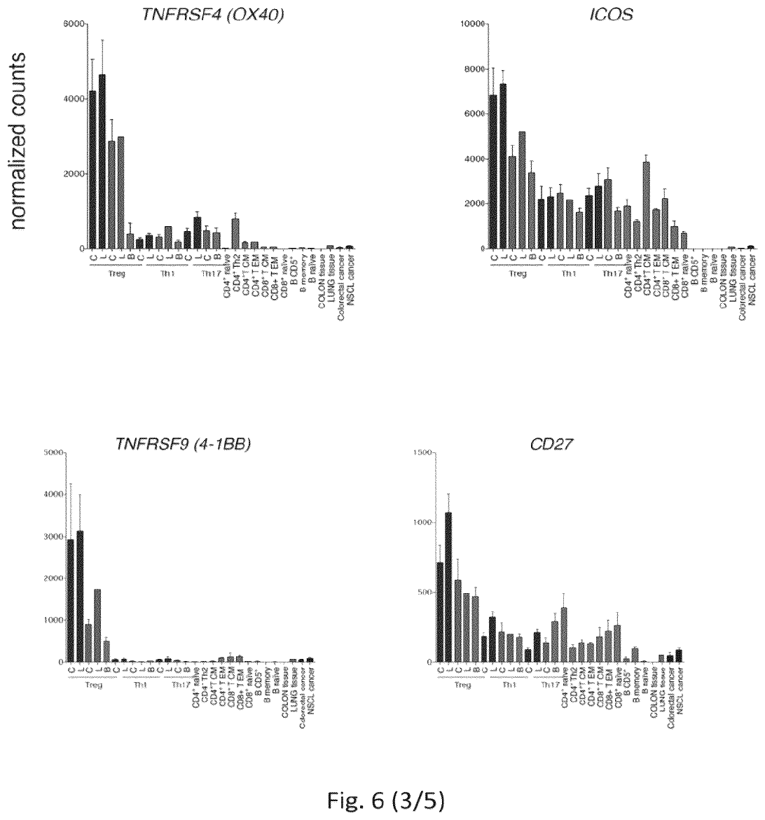

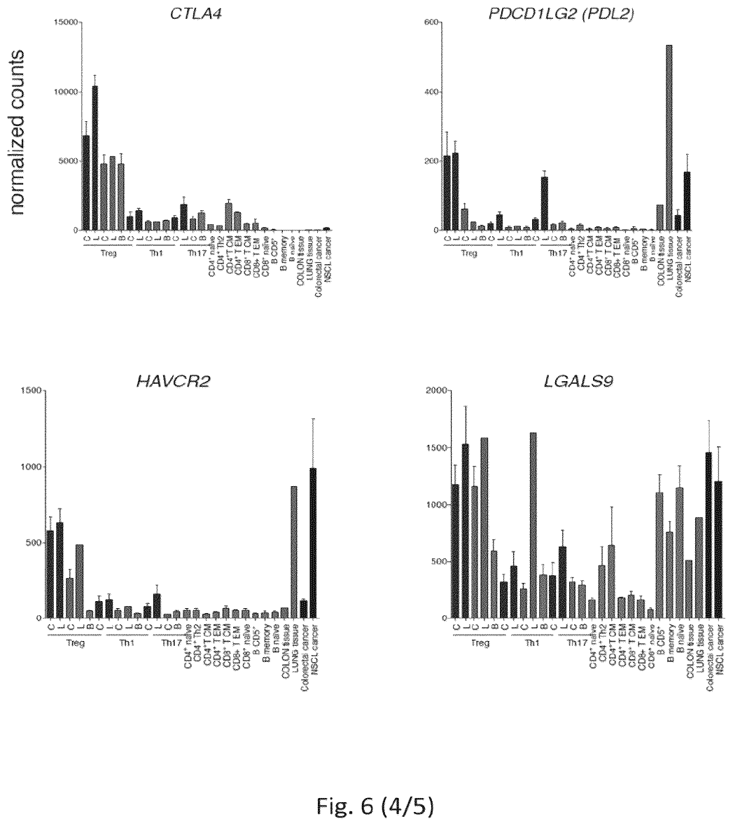

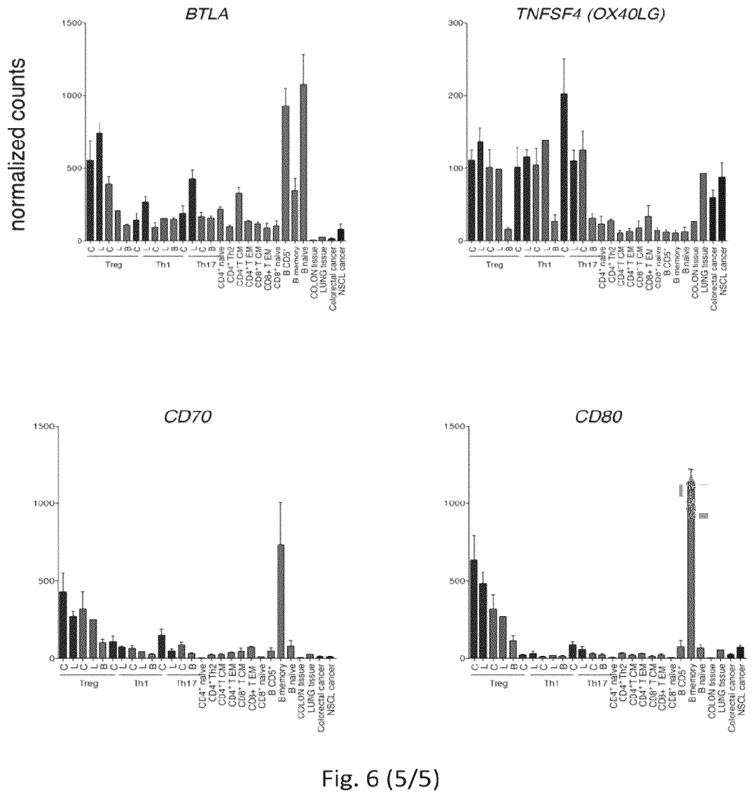

(C) Z-score normalized RNA-seq expression values of immunecheckpoints genes are represented as a heatmap. Cell populations are reported in the upper part of the graph, while gene names have been assigned to heatmap rows. Hierarchical clustering results are shown as a dendrogram drawn on the left side of the matrix. Colon tissues are indicated as C, lung tissues as L and peripheral blood as B. See also FIG. 6.

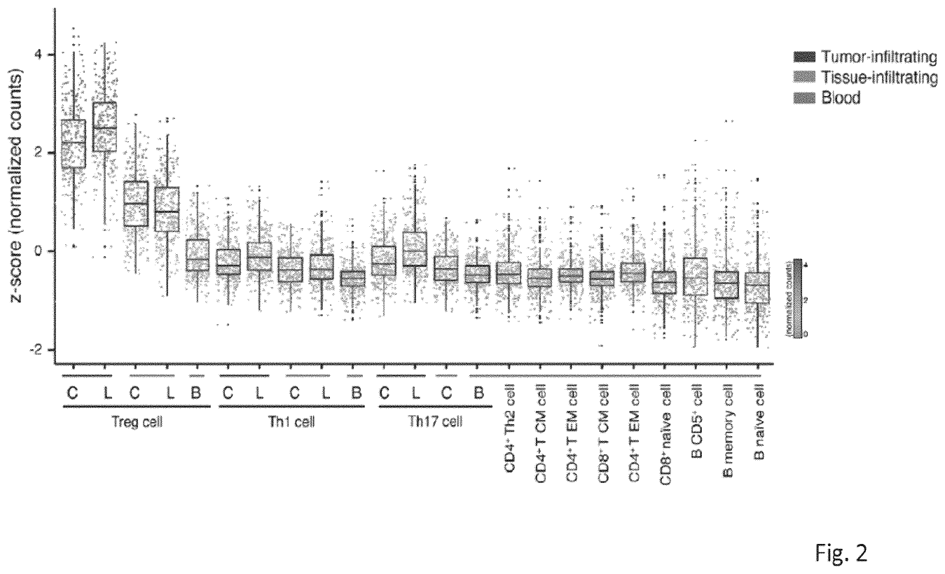

FIG. 2. Differential expression analysis identifies co-regulated genes in tumor infiltrating Treg cells

Z-score normalized expression values of genes that are preferentially expressed in tumor-infiltrating Tregs (Wilcoxon Mann Whitney test p<2.2×10−16) over the listed cell subsets are represented as boxed plots. Colon tissues are indicated as C, lung tissues as L and peripheral blood as B.

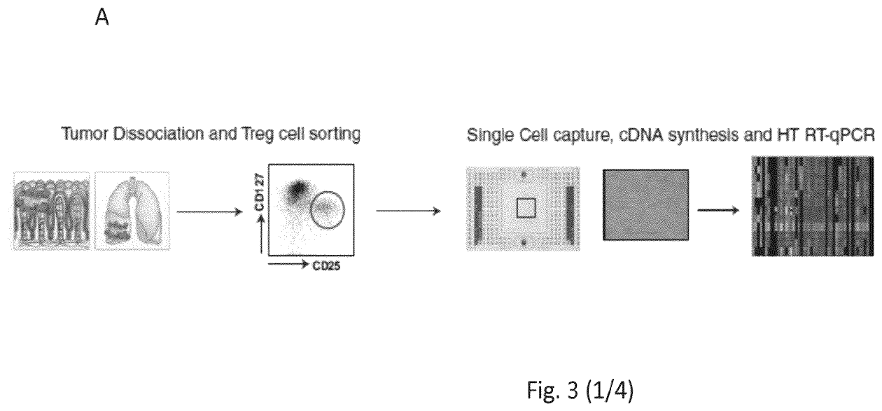

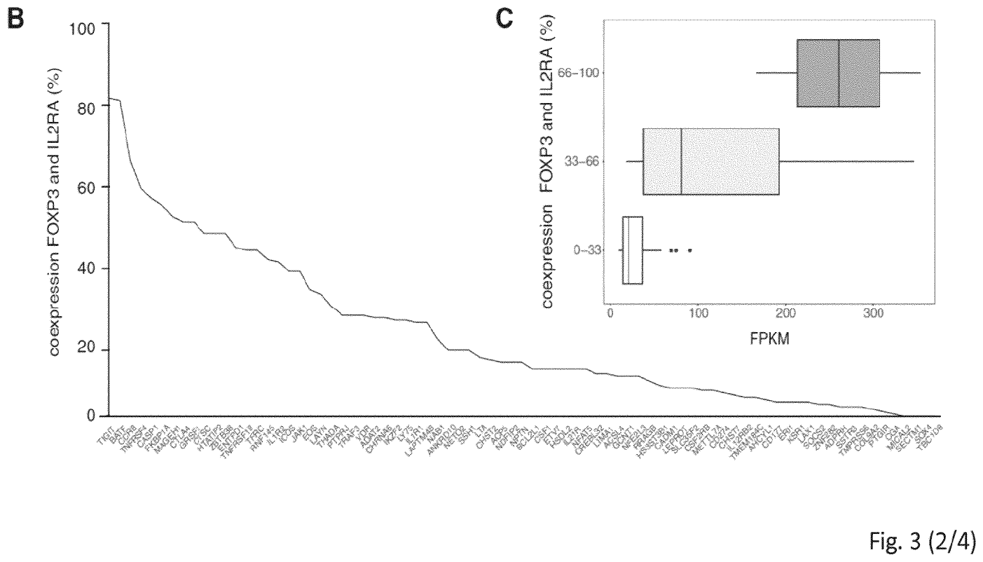

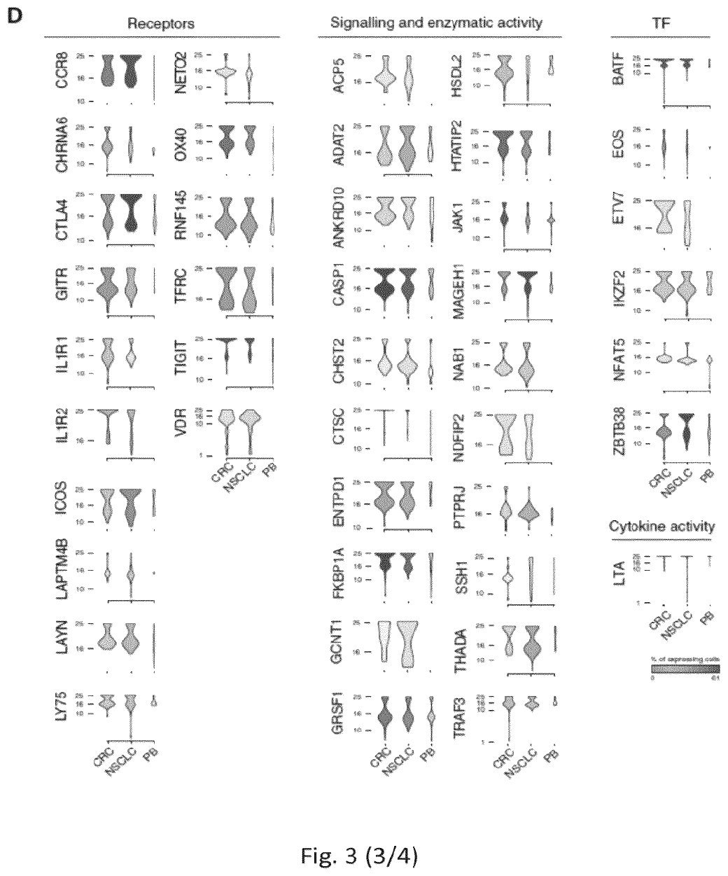

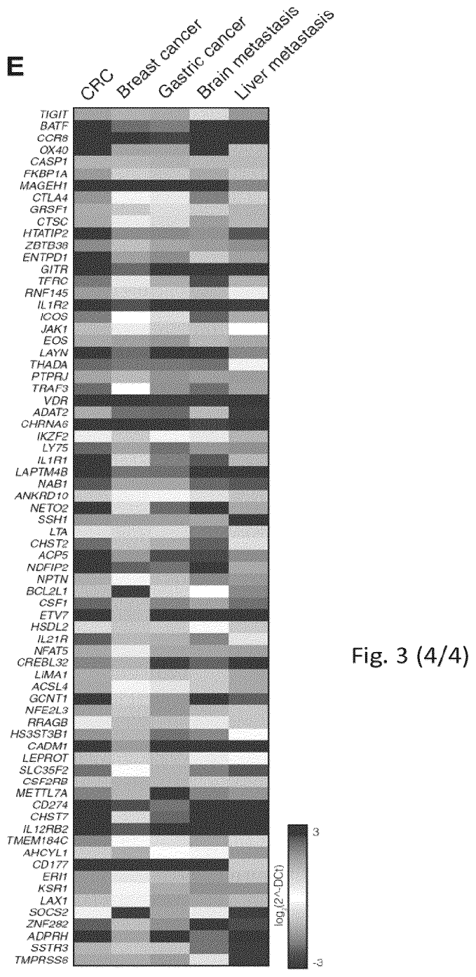

FIG. 3. Single cell analysis of tumor infiltrating Treg cells

(A) Schematic representation of the experimental workflow. Experiments were performed on Treg cells infiltrating CRC, NSCLC, or isolated from peripheral blood of healthy donors (PB); five samples were collected for each tissue.

(B) Percentage of co-expression of signature genes with FOXP3 and IL2RA is depicted.

(C) Expression levels of the signature genes classified by the percentage of co-expression are represented as box plot.

(D) Expression distribution (violin plots) in Treg cells infiltrating CRC, NSCLC or PB. Plots representing the ontology classes of receptors, signaling and enzymatic activity, cytokine activity and transcription factors are shown (Wilcoxon Mann Whitney test p<0.05). Gray scale gradient indicates the percentage of cells expressing each gene in Treg cells isolated from the three compartments.

(E) Gene expression analysis of tumor Treg signature genes in different tumor types. Expression values are expressed as log 2 (2\hat{ }−DCt).

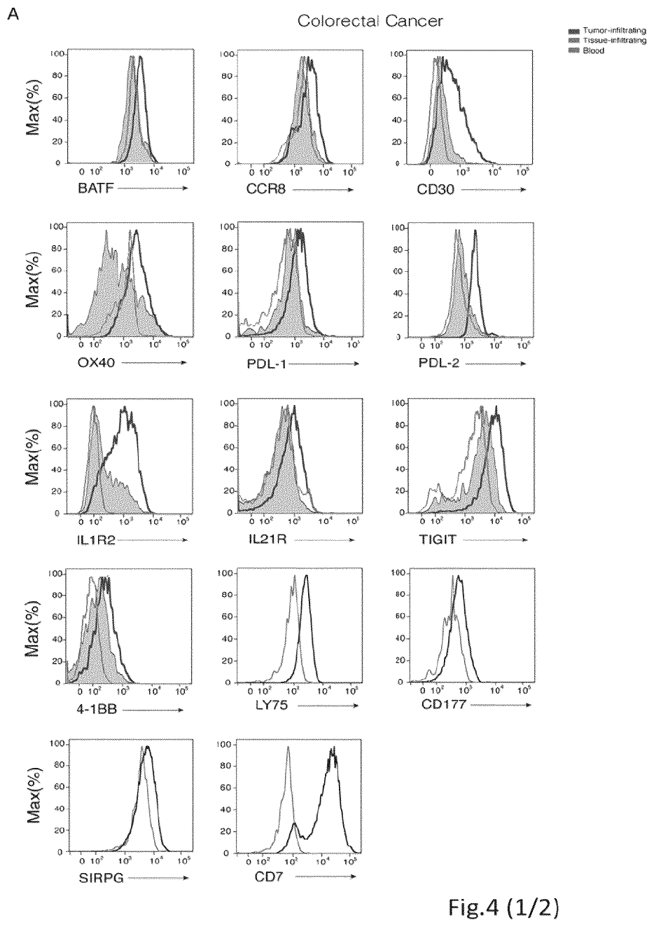

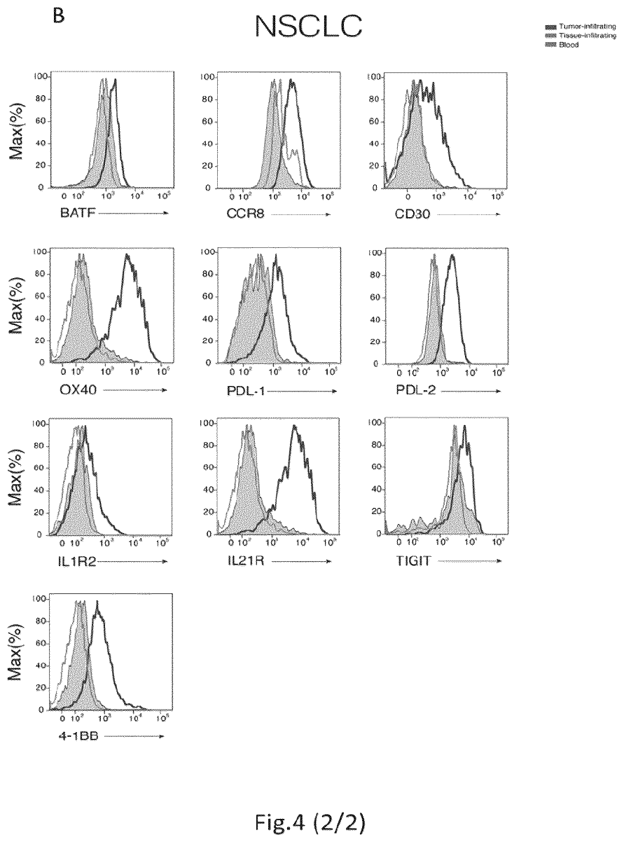

FIG. 4. Expression of tumor-infiltrating Treg cells protein signatures in CRC and NSCLC samples.

(A and B) Representative flow cytometry plots for tumor normal tissue infiltrating Treg cells and peripheral blood Treg cells analyzed for the expression of the indicated proteins.

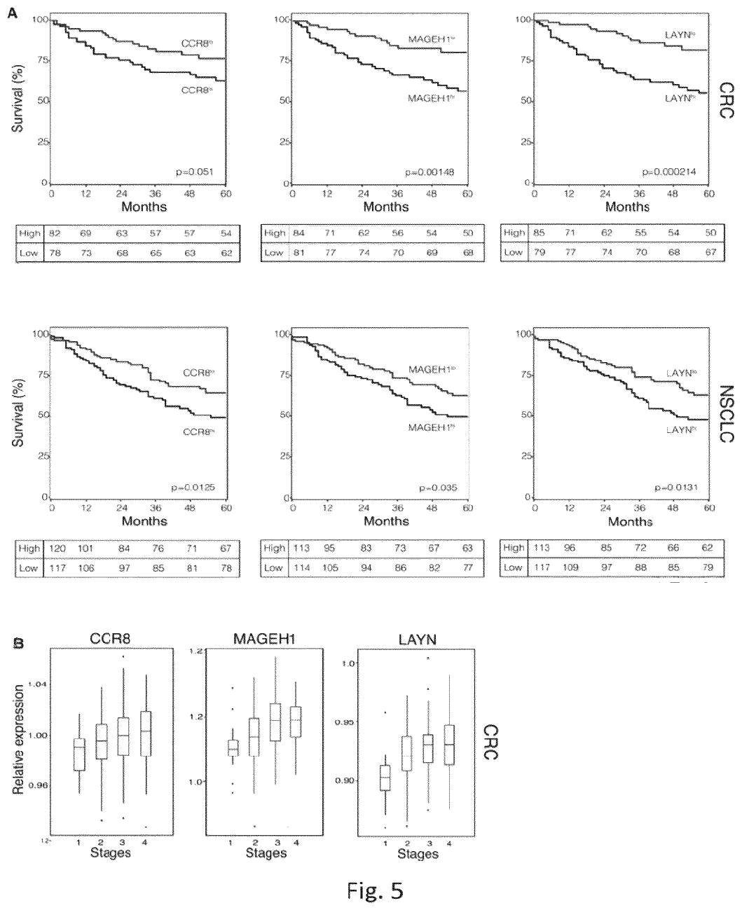

FIG. 5. Prognostic value of signature transcripts of tumor infiltrating Treg cells.

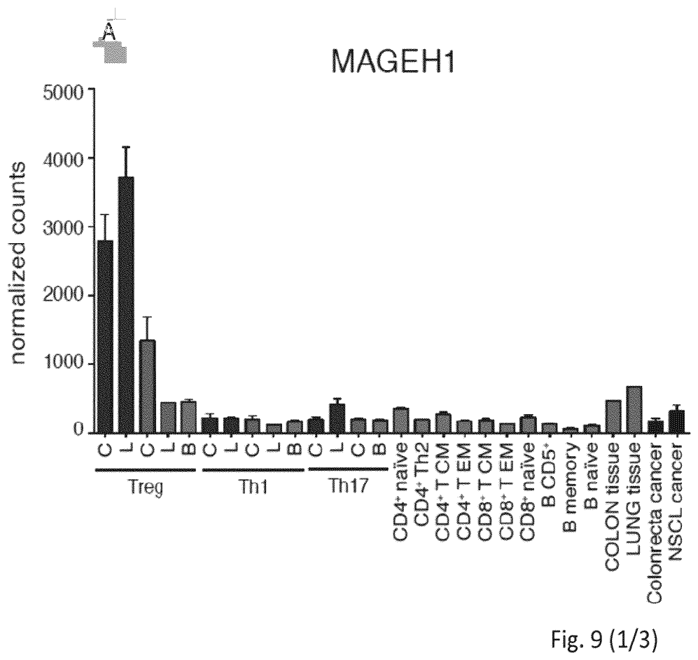

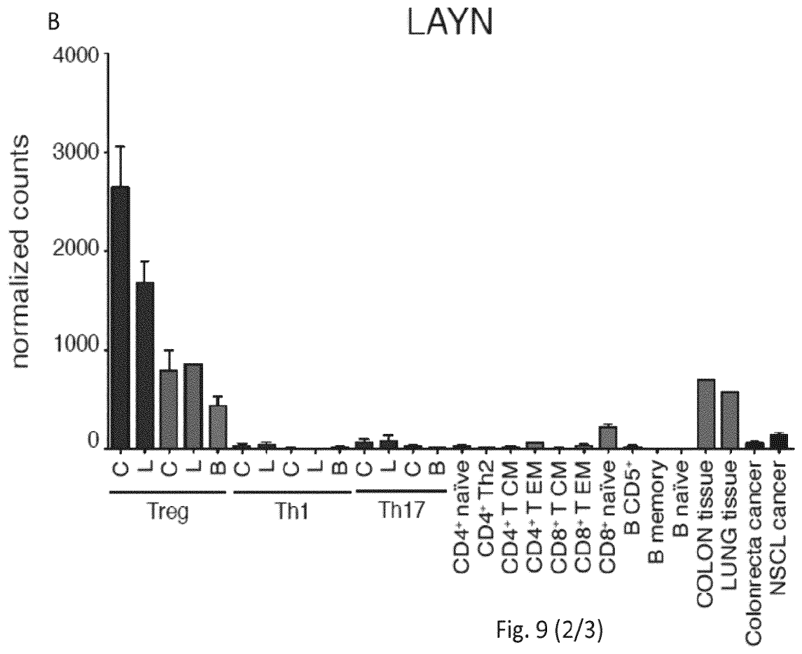

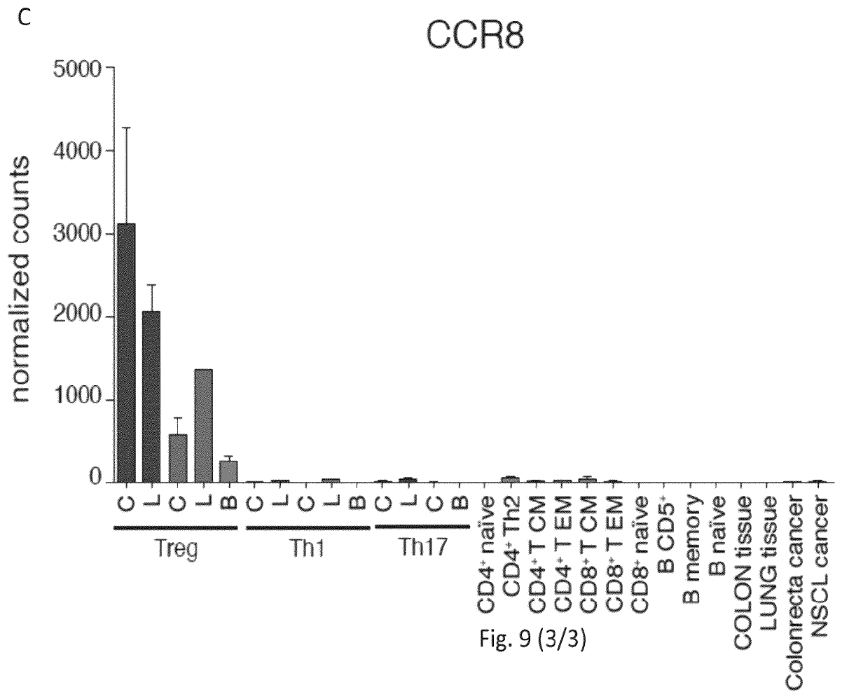

(A) Kaplan-Meier survival curve comparing the high and low expression of the tumor Treg signature transcripts (CCR8, MAGEH1, LAYN) normalized to the CD3G for the CRC (n=177) and NSCLC(n=263) studies. Univariate analysis confirmed a significant difference in overall survival curve comparing patients with high and low expression. Statistical significance was determined by the log-rank test. (CRC: p=0.05 for CCR8, p=1.48×10−3 for MAGEH1, p=2.1×10−4 for LAYN; NSCLC: p=0.0125 for CCR8, p=0.035 for MAGEH1, p=0.0131 for LAYN) Each table depicts the Kaplan Meier estimates at the specified time points. (B) Expression distributions of CCR8, MAGEH1 and LAYN according to tumor staging at the time of surgery in the cohort of CRC patients. See also FIG. 9.

FIG. 6 related to FIG. 1. Transcriptome analysis of tumor infiltrating lymphocytes.

(A) Representation of the sorting strategy of Treg cells infiltrating colorectal tumor or normal tissue.

(B) RNA-seq expression values (normalized counts) of FOXP3, TBX21 and RORC in CD4+ Th1, Th17 and Treg cells from CRC (C), NSCLC (L) or peripheral blood (PB) of healthy donors.

(C) RNA-seq normalized counts data for selected immune checkpoints and their ligands are shown as histogram plot. Cell population names are reported in the lower part of each graph, while gene names are shown in the upper part.

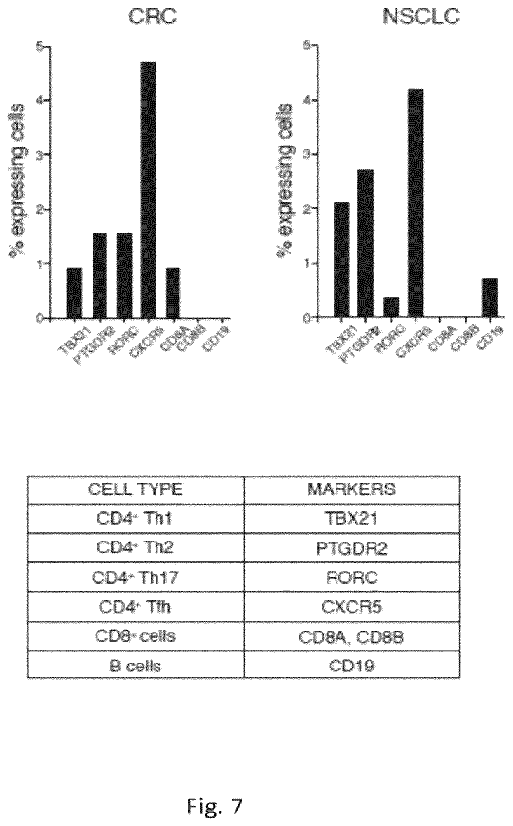

FIG. 7 related to FIG. 3. Single-cell analysis of tumor infiltrating Treg cells.

Assessment of CD4+ Treg, Th1, Th17, Th2, CD8+ T cells and B cell markers expression (percentage of expressing cells) in single Treg cells purified from NSCLC and CRC.

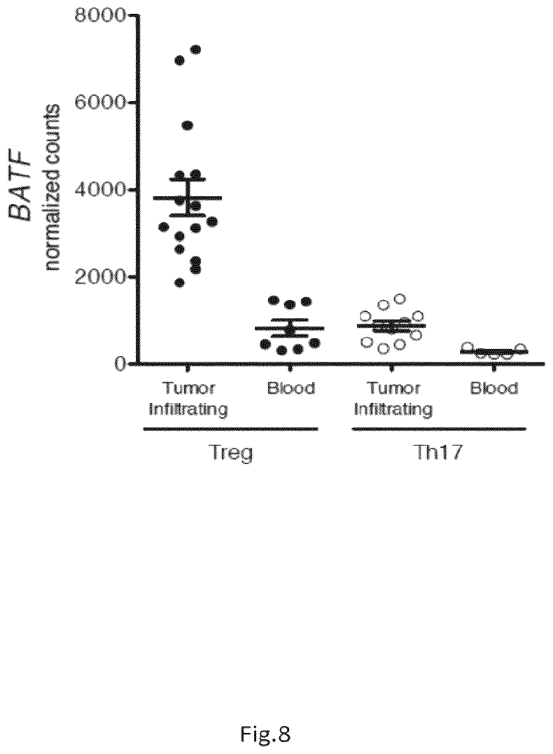

FIG. 8 related to FIG. 4. Comparison of BATF expression in CD4+ Treg vs Th17 cells.

BATF expression levels (RNA-seq normalized counts data) in CD4+ Treg and Th17 subsets isolated from tumor tissue or peripheral blood

FIG. 9 related to FIG. 5. Expression levels of tumour-infiltrating Treg signature genes.

RNA-seq normalized counts data of three tumour-infiltrating Treg signature genes (MAGEH1 (panel A), LAYN (panel B) and CCR8 (panel C)) across listed cell populations.

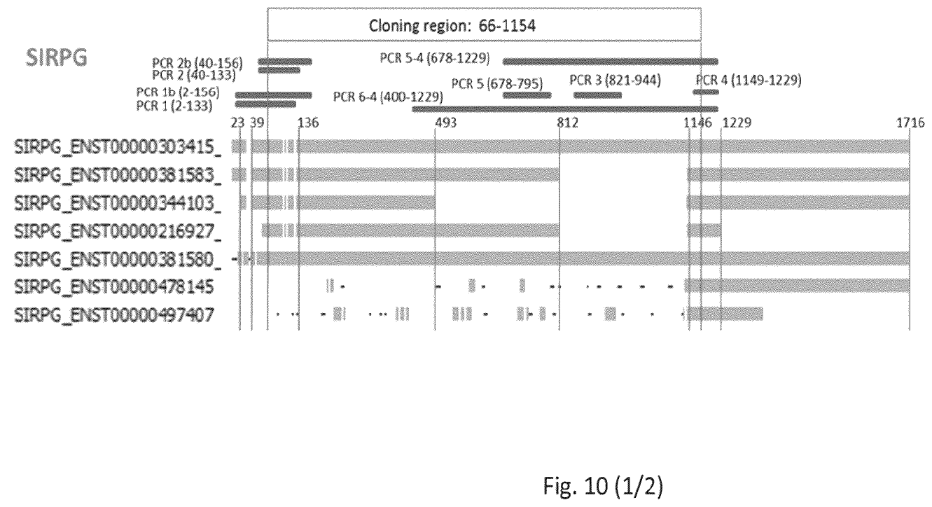

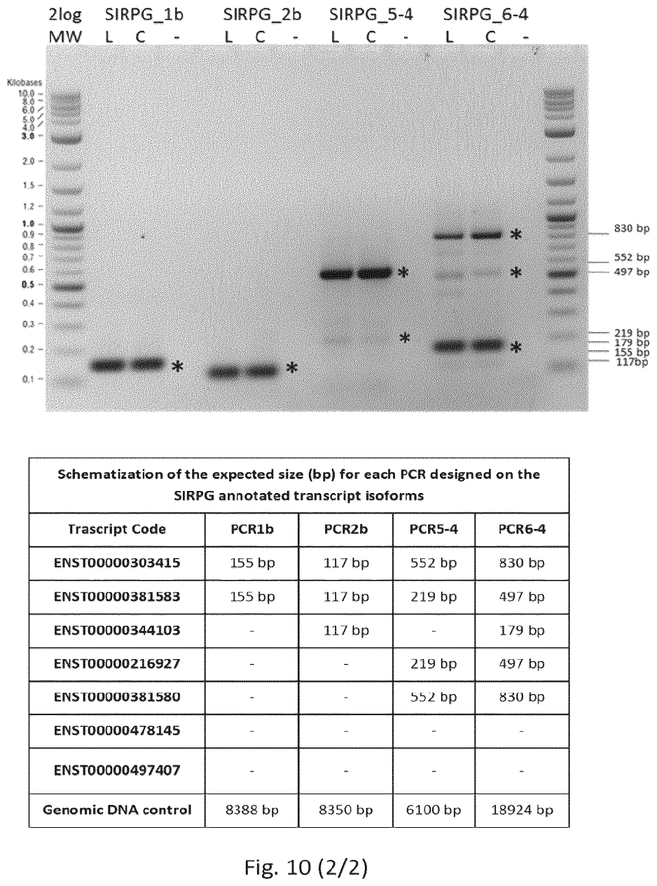

FIG. 10. Results of RT-PCR analysis done on cDNA from Tumor infiltrating Treg cells (L=NSCLC, C=CRC, −=ntc) with specific primers able to discriminate the different transcript isoforms annotated for SIRPG.

### DETAILED DESCRIPTION OF THE INVENTION

### Experimental Procedures

### Human Primary Tissues

Primary human lung or colorectal tumors and non-neoplastic counterparts were obtained respectively from fifteen and fourteen patients who underwent surgery for therapeutic purposes at Fondazione IRCCS Ca' Granda, Policlinico or San Gerardo Hospitals (Italy).

Records were available for all cases and included patients' age at diagnosis, gender, smoking habit (for lung cancer patients), clinicopathological staging (Sobin et al., 2009), tumor histotype and grade (Table II). No patient received palliative surgery or neoadjuvant chemo- and/or radiotherapy. Informed consent was obtained from all patients, and the study was approved by the Institutional Review Board of the Fondazione IRCCS Ca' Granda (approval n. 30/2014).

Non-small-cell lung cancer (NSCLC) were cut into pieces and single-cell suspensions were prepared by using the Tumor Dissociation Kit, human and the gentleMACS™ Dissociator (Miltenyi Biotech cat. 130-095-929) according to the accompanying standard protocol. Cell suspensions were than isolated by ficoll-hypaque density-gradient centrifugation (Amersham Bioscience). Colorectal cancer (CRC) specimens were cut into pieces and incubated in DTT 0.1 mM (Sigma-Aldrich) for 10 min, then extensively washed in HBSS (Thermo Scientific) and incubated in 1 mM EDTA (Sigma-Aldrich) for 50 min at 37° C. in the presence of 5% CO2. They were then washed and incubated in type D collagenase solution 0.5 mg/mL (Roche Diagnostic) for 4 h at 37° C. Supernatants containing tumor infiltrating lymphocytes were filtered through 100 μm cell strainer, centrifuged and fractionated 1800×g for 30 min at 4° C. on a four-step gradient consisting of 100%, 60%, and 40% and 30% Percoll solutions (Pharmacia). The T cell fraction was recovered from the inter-face between the 60% and 40% Percoll layers.

CD4 T cell subsets were purified by FACS sorting using the following fluorochrome conjugated antibodies: anti-CD4 APC/Cy7 (Biolegend clone OKT4), anti-CD27 Pacific Blue (Biolegend, clone M-T271), anti-IL7R PE (Milteniy, clone MB15-18C9), anti-CD25 PE/Cy7 (eBioscience, clone BC96), anti-CXCR3 PE/Cy5 (BD, clone 1C6/CXCR3), anti-CCR6 APC (Biolegend, clone G034E3) and anti-CCR5 FITC (Biolegend, clone j418F1) using a FACSAria II (BD).

### Flow Cytometry

To validate surface marker expression cells were directly stained with the following fluorochrome-conjugated antibodies and analyzed by flow cytometry: anti-CD4 (Biolegend, clone OKT4); anti-PD-L2 (Biolegend, Clone CL24F.10C12); anti-CD127 (eBioscience, clone RDR5); anti-BATF (eBioscience, clone MBM7C7), anti-GITR (eBioscience, clone eBIOAITR), anti-CD25 (Miltenyi, clone 4E3) and anti 4-1BB (eBioscience clone 4B4) anti CCR8(Biolegend clone L263G8) anti CD30 (eBioscience, clone Ber-H2) anti PD-L1 (Biolegend clone 29E.2A3) anti TIGIT (eBioscience, clone MBSA43) anti IL1 R2 (R and D clone 34141) IL21R (Biolegend clone 2G1-K12) anti OX40 (Biolegend clone Ber-ACT35). Intracellular staining was performed using eBioscience Foxp3 staining kit according to the manufactured's protocol (eBioscience cat 00-5523-00). Briefly cells were harvested and fixed for 30 min in fixation/permeabilization buffer at 4° C., and than stained with anti-FOXP3 antibody (eBioscience, clone 236A/E7) and anti-BATF (eBioscience clone MBM7C7) in permeabilisation buffer for 30 min at 4° C. Cells were then washed two times, resuspended in FACS washing buffer and analyzed by flow cytometry.

### Suppression Assay.

4×10⁴ carboxyfluorescein diacetate succinimidyl ester (CFSE)-labeled (1 μM) responders Naive⁺ T cells from healthy donors were cocultured with different E/T ratio with unlabeled CD127⁻CD25^{low}CD4⁺ T cells sorted from TILs or PBMCs of patients with CRC or NSCLC, using FACS Aria II (BD Biosciences), in the presence of CD11c⁺CD1c⁺dentritic cells as antigen-presenting cells and 0.5 mg/ml anti-CD3 (OKT3) mAb. Proliferation of CFSE-labeled cells was assessed by flow cytometry after 96 hr culture. RNA Isolation and RNA Sequencing

RNA from tumor-infiltrating lymphocytes was isolated using mirVana Isolation Kit.

Residual contaminating genomic DNA was removed from the total RNA fraction using Turbo DNA-free (Thermo Fisher). The RNA yields were quantified using the QuantiFluor RNA System (Promega) and the RNA quality was assessed by the Agilent 2100 Bioanalyzer (Agilent). Libraries for Illumina sequencing were constructed from 50 ng of total RNA with the Illumina TruSeq RNA Sample Preparation Kit v2 (Set A). The generated libraries were loaded on to the cBot (Illumina) for clustering on a HiSeq Flow Cell v3. The flow cell was then sequenced using a HiSeq 2500 in High Output mode (Illumina). A paired-end (2×125) run was performed.

### RNA-Seq Data Analysis

Raw .fastq files were analyzed using FastQC v0.11.3, and adapter removal was performed using cutadapt 1.8. Cutadapt is run both for reverse and forward sequences with default parameters [-anywhere <adapter1>-anywhere <adapter2>-overlap 10-times 2-mask-adapter]. Adapter sequences used for libraries preparation are

Adapter1:
(SEQ ID NO: 710)
AGATCGGAAGAGCACACGTCTGAACTCCAGTCACNNNNNNATCTCGTATG
CCGTCTTCTGCTTG
Adapter2:
(SEQ ID NO: 711)
AGATCGGAAGAGCGTCGTGTAGGGAAAGAGTGTAGATCTCGGTGGTCGCC
GTATCATT

Trimming was performed on raw reads using Trimmomatic (Bolger et al., 2014): standard parameters for phred33 encoding were used: ILLUMINACLIP (LEADING:3 TRAILING:3 SLIDINGWINDOW:4:15), MINLEN parameter was set to 50.

Mapping and quantification: reads mapping to the reference genome (GRCh38) was performed on quality-checked and trimmed reads using STAR 2.4.1c: [STAR-genomeDir <index_star>-runThreadN <cpu_number>-readFilesin <trimmed>_R1.fastq.gz<trimmed>_R2_P.fastq.gz-readFilesCommand zcat]. The reference annotation is Ensembl v80. The overlap of reads with annotation features found in the reference .gtf was calculated using HT-seq v0.6.1. The output computed for each sample (raw read counts) was then used as input for DESeq2 analysis. Raw counts were normalized using DESeq2's function ‘rlog’, and normalized counts were used to perform and visualize Principal Component Analysis (PCA) results (using DESeq2's ‘plotPCA’ function).

Differential expression analysis: differential expression analyses of tumor-infiltrating CD4+ Treg/Th1/Th17 subsets vs. CD4+ Treg/Th1/Th17 from PBMC were performed using DESeq2. Upregulated/downregulated genes were selected for subsequent analyses if their expression values were found to exceed the threshold of 0.05 FDR (Benjamini-Hochberg correction).

Capturing of Single Cells, Preparation of cDNA and Single-Cell PCR

Treg cells from 5 CRC and 5 NSCLC specimens were isolated as previously described (See also Table II).Single cells were captured on a microfluidic chip on the C1 System (Fluidigm) and whole-transcriptome amplified. cDNA was prepared on chip using the SMARTer Ultra Low RNA kit (Clontech). Cells were loaded onto the chip at a concentration of 3-5E5 cells/ml, stained for viability (LIVE/DEAD cell viability assay; Thermo Fisher) and imaged by phase-contrast and fluorescence microscopy to assess the number and viability of cells per capture site. Only single, live cells were included in the analysis. For qPCR experiments, harvested cDNA was pre-amplified using a 0.2× pool of primers prepared from the same gene expression assays to be used for qPCR.

Pre-amplification allows for multiplex sequence-specific amplification 78 targets. In detail, a 1.25 μl aliquot of single cell cDNA was pre-amplified in a final volume of 5 μl using 1 μl of PreAmp Master Mix (Fluidigm) and 1.25 μl pooled TaqMan assay mix (0.2×). cDNA went through amplification by denaturing at 95° C. for 15 s, and annealing and amplification at 60° C. for 4 min for 20 cycles. After cycling, pre-amplified cDNA was diluted 1:5 by adding μl TE Buffer to the final 5 μl reaction volume for a total volume of 25 μl. Single-cell gene expression experiments were performed using the 96×96 quantitative PCR (qPCR) DynamicArray microfluidic chips (Fluidigm). A 2.25 μl aliquot of amplified cDNA was mixed with 2.5 μl of TaqMan Fast Adavanced Master Mix (Thermo Fisher) and 0.25 μl of Fluidigm's “sample loading agent,” then inserted into one of the chip “sample” inlets. A 2.5 μl aliquot of each 20×TaqMan assay was mixed with 2.5 μl of Fluidigm's “assay loading agent” and individually inserted into one of the chip “assay” inlets. Samples and probes were loaded into 96×96 chips using an IFC Controller HX (Fluidigm), then transferred to a BioMark real-time PCR reader (Fluidigm) following manufacturer's instructions. A list of the 78 TaqMan assays used in this study is provided below.

TABLE V
Related to FIG. 3.
List of TaqMan Probes and assay number
used in RT-qPCR single-cell experiments
Taqman Assays Numbers

| | Assay | | |
|---|---|---|---|
| Gene Name | Number | Gene Name | Assay Number |
| BCL2L1 | Hs00235329_m1 | ACP5 | Hs00356261_m1 |
| EOS | Hs00223842_m1 | BATF | Hs00232390_m1 |
| AHCYL1 | Hs00198382_m1 | SLC35F2 | Hs00233850_m1 |
| NFE2L3 | Hs00852569_m1 | LAX1 | Hs00214948_m1 |
| IL12RB2 | Hs00155486_m1 | CCR8 | Hs00174764_m1 |
| CD177 | Hs00360669_m1 | ADPRH | Hs00153890_m1 |
| OX40 | HS00937194_g1 | IKZF2 | Hs00212361_m1 |
| METTL7A | Hs00204042_m1 | C5F2RB | Hs00166144_m1 |
| ENTPD1 | HS00969339_m1 | NDFIP2 | Hs00324851_m1 |
| NFAT5 | Hs00232437_m1 | CADM1 | Hs00942508_m1 |
| CT9C | Hs00175188_m1 | ICOS | Hs00359999_m1 |
| SSH1 | Hs00368014_m1 | COL9A2 | Hs00156712_m1 |
| TMEM184C | Hs00217311_m1 | LTA | Hs00236874_m1 |
| HTATIP2 | Hs03091727_m1 | MAGEH1 | Hs00371974_s1 |
| HSDL2 | Hs00953689_m1 | IL21R | Hs00222310_m1 |
| FOXP3 | Hs01085834_m1 | S6TR3 | Hs01066399_m1 |
| IL2RA | Hs00907778_m1 | RNF145 | Hs01066399_m1 |
| LIMA1 | Hs01033646_m1 | LAPTM4B | Hs00363282_m1 |
| NAB1 | Hs00428619_m1 | GRSF1 | Hs00909877_m1 |
| ACSL4 | Hs00244871_m1 | ANKRD10 | Hs00214321_m1 |
| ERI1 | Hs00405251_m1 | NPTN | Hs01033353_m1 |
| FKEP1A | Hs00356621_g1 | HS3ST3B1 | Hs00797512_s1 |
| LEPROT | Hs00956627_s1 | TRAF3 | Hs00936781_m1 |
| NETO2 | Hs00983152_m1 | RRAGB | Hs01099787_m1 |
| VDR | Hs00172113_m1 | ZBT3S | Hs00257315_s1 |
| CSF1 | Hs00174164_m1 | TIGIT | Hs00545087_m1 |
| GITR | Hs00188346_m1 | TFRC | Hs00951083_m1 |
| IL1R2 | Hs01030384_m1 | JAK1 | Hs01026982_m1 |
| IL1R1 | Hs00991010_m1 | KSR1 | Hs00300134_m1 |
| LAYN | Hs00379511_m1 | ZNF202 | Hs00411965_m1 |
| THADA | Hs00736554_m1 | PTPRJ | Hs01119326_m1 |
| CTLA4 | Hs00175480_m1 | CHRNA6 | Hs02563909_s1 |
| CHST2 | Hs01921028_s1 | IL2RB | Hs01081597_m1 |
| CHST7 | Hs00219871_m1 | TBX21 | Hs00203436_m1 |
| LRBA | Hs01032231_m1 | RORC | Hs01076112_m1 |
| ETV7 | Hs00903229_m1 | CXCR5 | Hs00540548_s1 |
| LY75 | Hs00982383_m1 | CD8A | Hs00233520_m1 |
| ADAT2 | Hs00699339_m1 | CD8B | Hs00174762_m1 |
| GCNT1 | Hs00155243_m1 | PTGDR2 | Hs00173717_m1 |
| CASP1 | Hs00354836_m1 | CD19 | Hs01047410_g1 |

Single-cell data analysis: The Quality Threshold in the BioMark™ Analysis software is a qualitative tool designed to measure the “quality” of each amplification curve. Basically, each amplification curve is compared to an ideal exponential curve and as the quality score approaches 1 the closer it is to ideal. The further the curve is from ideal, its quality score approaches 0. The default cutoff of 0.65 is an arbitrary value set by Fluidigm. Any curve above 0.65 passes. Any curve below, fails. Baseline correction was set on Linear (Derivative)[default]. Ct Threshold Method was set on Auto (Detectors). This method independently calculates a threshold for each detector on a chip. For clustering and downstream analysis, raw Cts have been converted to Log 2Exp by using a Limit of Detection (LOD) of 35, which corresponds to the last PCR cycle. Co-expression analysis has been performed by considering both CRC and NSCLC samples on those genes for which both FOXP3 and IL2RA were co-expressed at least to 2%. Gene's levels above the background were depicted as violin plots after log 2 scale transformation by ggplot2 (v. 2.1.10). The violin color gradient is the percentage of cells that are expressing the gene of interest and the upper bound of the color scale is the maximum percentage of cells that express a gene of the whole geneset.

Procedure for the removal of transcripts whose expression values are affected by the ‘dropout’ effect. Single-cell qPCR data are inherently noisy, and due the limitations of current technologies the expression patterns of a certain number of genes may be affected by the ‘dropout effect’. Inventors performed a gene selection procedure in order to take into account this ‘dropout’ effect and discard those genes whose expression values cannot be reliably used in a binary comparison (tumor-peripheral vs blood). Inventors fitted a number of parametric distributions to the ratios of detected genes on the total number of tumor cells (both NSCLC and CRC) and selected the reciprocal inverse Gaussian continuous random variable as best fit.

Inventors then calculated the median value of the fitted distribution and discarded those genes whose detection ratio is less than this threshold value (at least 8.4% of detection). Inventors reasoned that these genes are more likely to be affected by the ‘dropout’ effect. With this threshold inventors selected 45 genes for which a non-parametric T-test (Wilcoxon Mann Whitney test p<0.05) has been performed (by comparing tumor vs. peripheral blood samples).

### Meta Analysis Kaplan-Meier and Stage Correlation

Statistical analysis was performed by using the R survival package (Therneau T. 2013). Survival times were calculated as the number of days from initial pathological diagnosis to death, or the number of days from initial pathological diagnosis to the last time the patient was reported to be alive. The Kaplan-Meier (KM) was used to compare the high and low expression levels of the tumor-Treg cell signature transcripts in either CRC (GSE17536) and NSCLC (GSE41271) patients. For both studies annotation was normalized to four tumor stages (1,2,3,4). For study GSE41271 five patients were excluded due to incomplete or inaccurate annotation (GSM1012883, GSM1012884, GSM1012885, GSM1013100, GSM1012888), retaining a total of two hundred and sixty three patients. Patients from both studies were labeled as ‘High’ ‘Low’ whether or not their relative expression values exceeded a decision boundary (mean of the samples). Inventors define \ddot{x}ᵢⱼ to denote the relative expression of the gene i for the n samples of the study normalized to the CD3 level:

$${\tilde{x}}_{i , j} = \frac{{x}_{i , j}}{{x}_{CD 3 G , j}} ; \qquad i = ( CCR 8 , MAGEH 1 , LAYN ) \qquad j = 1 , 2 , … , n samples$$

To classify a patient, a threshold on the \ddot{x}ᵢⱼ is required and defined as

$${T}_{( Upper Lower )} = median ( {\tilde{x}}_{i , j} ) \pm \frac{σ ( {\tilde{x}}_{i , j} )}{10}$$

where T_{(Upper,Lower)} represent the upper and lower extreme of the decision boundary:

$$\{ \begin{matrix}{\tilde{x}}_{i , j} > {T}_{Upper} High \\ {\tilde{x}}_{i , j} < {T}_{Lower} Low \\ {T}_{Upper} \leq {\tilde{x}}_{i , j} \leq {T}_{Lower} excluded\end{matrix}$$

Inventors examined the prognostic significance of tumor Treg cells transcripts by using log-rank statistics; a p-value of less than 0.05 was considered statistically significant. Since the log-rank test resulted in a p-value of less than 0.05, a post stage comparison by means of box plot representation was performed in order to evaluate the correlation degree between the expression level of the transcripts and tumor stages in the cohort of CRC patients. The annotation was normalized to four tumor stages (1,2,3,4).

### ACCESSION NUMBERS

The accession numbers for the present data are as follows: ENA: PRJEB11844 for RNA-seq tumor and tissue infiltrating lymphocytes; ArrayExpress: E-MTAB-2319 for RNA-seq human lymphocytes datasets; ArrayExpress: E-MTAB-513 for Illumina Human BodyMap 2.0 project; GEO: GSE50760 for RNA-seq datasets CRC; GEO: GSE40419 for RNA-seq datasets NSCLC; GEO: GSE17536 for CRC expression profiling by array; and GEO: GSE41271 for NSCLC expression profiling by array.

### Prediction of Surface-Exposed and Membrane-Associated Proteins

The probability of surface exposure of the proteins encoded by the genes of interest was determined by a combination of four different cell localization prediction algorithms: Yloc (Briesemeister et al, 2010), TMHMM (http://www.cbs.dtu.dk/services/TMHMM/), SignalP (http://www.cbs.dtu.dk/services/SignalP/) and Phobius (KAII et. al., 2007). In particular Yloc is a interpretable system offering multiple predictive models in animal version; inventors used both YLoc-LowRes predicting into 4 location (nucleus, cytoplasm, mitochodrion, secretory pathway) and Yloc-HighRes predicting into 9 locations (extracellular space, plasma membrane, nucleus, cytoplasm, mitochodrion, endoplasmic reticulum, peroxisome, Golgi apparatus, and lysosome).

TMHMM and SignalP were developed by the bioinformatic unit of the technical University of Denmark for the prediction of transmembrane helices and the presence and location of signal peptide cleavage sites in amino acid sequences, respectively. Phobius is a combined transmembrane topology and signal peptide predictor.

### RT-PCR Analysis of Transcript Isoforms Expressed by Tumor-Infiltrating Regulatory T Cells (Treg Cells)

Total RNA was extracted from tumor Treg cells (NSCLC or CRC) using miRCURY RNA isolation kit (Exiqon) and 1 μg was reverse transcribed with iScript reverse transcription supermix (BIORAD). Afterwards, 25 ng of cDNA were amplified with DreamTaq Green PCR Master Mix (ThermoScientific) using multiple gene-specific primers able to discriminate the different isoforms. PCR products were run on agarose gel. The expression of specific transcripts was assessed based on the expected band size.

### Results

### Tumor Infiltrating Tregs Cells Upregulate Immune Checkpoints and are Highly Suppressive

To assess the gene expression landscape of tumor infiltrating CD4⁺ T cells, the inventors isolated different CD4⁺ lymphocytes subsets from two different tumors, NSCLC and CRC, from the adjacent normal tissues, and from peripheral blood samples. From all these tissues, the inventors purified by flow cytometry (FIGS. 1A and 6A and 6B) CD4⁺ Treg (36 samples from 18 individuals), Th1 (30 samples from 21 individuals) and Th17 (22 samples from 14 individuals) cells (Table I and Table II).

TABLE 1
Purification and RNA-Sequencing of Human Primary Lymphocyte Subsets

| | | | | Mapped |
|---|---|---|---|---|
| | | Sorting | Number of | Reads |
| Tissue | Subset | Phenotype | Samples | (M) |
| NSCLC | CD4+ Treg | CD4⁺ CD127⁻ CD25⁺ | 8 | 587 |
| | CD4⁺ Th1 | CD4⁺ CXCR3⁺ CCR6⁻ | 8 | 409 |
| | CD4⁺ Th17 | CD4⁺ CCR6⁺ CXCR3⁻ | 6 | 206 |
| CRC | CD4⁺ Treg | CD4⁺ CD127⁻ CD25⁺ | 7 | 488 |
| | CD4⁺ Th1 | CD4⁺ CXCR3⁺ CCR6⁻ | 5 | 266 |
| | CD4⁺ Th17 | CD4⁺ CCR6⁺ CXCR3− | 5 | 308 |
| Lung | CD4⁺ Treg | CD4⁺ CD127⁻ CD25⁺ | 1 (pool | 73 |
| (normal tissue) | | | of 6) | |
| | CD4⁺ Th1 | CD4⁺ CXCR3⁺ CCR6⁻ | 1 (pool | 76 |
| | | | of 6) | |
| Colon | CD4⁺ Treg | CD4⁺ CD127⁻ CD25⁺ | 7 | 404 |
| (normal tissue) | CD4⁺ Th1 | CD4⁺ CXCR3⁺ CCR6⁻ | 6 | 352 |
| | CD4⁺ Th17 | CD4⁺ CCR6⁺ CXCR3⁻ | 6 | 284 |
| PB (healthy donor) | CD4⁺ Treg | CD4⁺ CD127⁻ CD25⁺ | 8 | 259 |
| | CD4⁺ Th1 | CD4⁺ CXCR3⁺ CCR6⁻ | 5 | 70 |
| | CD4⁺ Th17 | CD4⁺ CCR6⁺ CXCR3⁻ | 5 | 77 |

For each cell subsets profiled by RNA-sequencing tissue of origin, surface marker combinations used for sorting, number of profiled samples, as well as number of mapped sequencing reads are indicated. M, million; CRC, colorectal cancer; NSCLC, non-small cell lung cancer; PB, peripheral blood.

TABLE II
Table II related to Table I. Patients information and histological analysis.
For each cell subset profiled by RNA-sequencing, patient records are shown including: age at diagnosis,
gender, smoking habit (for lung cancer patients), clinicopathological staging (TNM classification) tumor
histotype and grade. For Treg cell isolated for qPCR experiment the same information are available,
including also the number of live cells captured from each tumor and available for single-cell analysis.

| NSCLC | | | | | | | | | |
|---|---|---|---|---|---|---|---|---|---|
| PATIENTS | | | | | | | | | |
| LIST (RNA | | | | | | | SMOKE | | |
| SEQUENCING) | (T)Th1 | (T)Th17 | (T)Treg | (H)Th1 | (H)Th17 | (H)Treg | HABIT | STATUS | GENDER |
| PATIENT1 | | | SQ_0342 | | | | PREVIOUS | ALIVE | M |
| | | | | | | | SMOKER | | |
| | | | | | | | >15 y | | |
| PATIENT2 | | | SQ_0339 | | | | PREVIOUS | ALIVE | M |
| | | | | | | | SMOKER | | |
| | | | | | | | <15 y | | |
| PATIENT3 | SQ_0365 | SQ_0375 | | | | | PREVIOUS | ALIVE | M |
| | | | | | | | SMOKER | | |
| | | | | | | | <15 y | | |
| PATIENT4 | SQ_0366 | SQ_0374 | SQ_0341 | | | | PREVIOUS | ALIVE | M |
| | | | | | | | SMOKER | | |
| | | | | | | | >15 y | | |
| PATIENT5 | SQ_0364 | SQ_0373 | | SQ_0350 | | SQ_0351 | SMOKER | DEAD | M |
| PATIENT6 | SQ_0358 | | SQ_0336 | SQ_0350 | | SQ_0351 | PREVIOUS | ALIVE | M |
| | | | | | | | SMOKER | | |
| | | | | | | | <15 y | | |
| PATIENT7 | SQ_0363 | SQ_0376 | SQ_0334 | SQ_0350 | | SQ_0351 | PREVIOUS | ALIVE | M |
| | | | | | | | SMOKER | | |
| | | | | | | | <15 y | | |
| PATIENT8 | SQ_0357 | | SQ_0337 | SQ_0350 | | SQ_0351 | PREVIOUS | ALIVE | M |
| | | | | | | | SMOKER | WITH | |
| | | | | | | | <15 y | RELAPSE | |
| PATIENT9 | SQ_0404 | SQ_0408 | SQ_0398 | SQ_0350 | | SQ_0351 | SMOKER | ALIVE | F |
| PATIENT10 | SQ_0403 | SQ_0407 | SQ_0396 | SQ_0350 | | SQ_0351 | PREVIOUS | ALIVE | F |
| | | | | | | | SMOKER | | |
| | | | | | | | >15 y | | |

| NSCLC | | | ADCA | | | | | |
|---|---|---|---|---|---|---|---|---|
| PATIENTS | | HISTO- | SUBTYPE | | | | | |
| LIST (RNA | | TYPE | (PRE- | | pTNM: | pTNM: | pTNM: | |
| SEQUENCING) | AGE(y) | MAJOR | DOMINANT) | GRADE | T | N | M | STAGE |
| PATIENT1 | 84 | SCC | | G3 | 2b | 0 | 0 | IIA |
| PATIENT2 | 83 | SCC | | G3 | 2a | 0 | 0 | IB |
| PATIENT3 | 72 | SCC | | G3 | 2 | 2 | 0 | IIIA |
| PATIENT4 | 79 | SCC | | G3 | 2a | 0 | 0 | IB |
| PATIENT5 | 66 | SCC | | G3 | 3 | 2 | 0 | IIIA |
| PATIENT6 | 71 | SCC | | G3 | 4 | 1 | 0 | IIIA |
| PATIENT7 | 78 | SCC | | G3 | 2b | 1 | 0 | IB |
| PATIENT8 | 77 | ADCA | SOLID | G3 | 2 | 2 | 0 | IIIA |
| PATIENT9 | 69 | ADCA | SOLID | G3 | 1a | 0 | 0 | IA |
| PATIENT10 | 77 | ADCA | ACINAR | G3 | 1a | 0 | 0 | IA |

NSLC = Non Small Cell Lung Cancer
ADC = Adenocarcinoma
SCC = Squamous Cell Carcinoma
(T) = Tumor Sample
(H) = Healthy Tissue

| TUMOR | | | | | | |
|---|---|---|---|---|---|---|
| INFILTRATING | | | | | | |
| TREG FROM | | | | | | |
| NSCLC | | | | | HISTO- | |
| (SINGLE | SMOKE | | | | TYPE | ADCA SUBTYPE |
| CELL qPCR) | HABIT | STATUS | GENDER | AGE(y) | MAJOR | PREDOMINANT |
| PATIENT1 | NEVER | ALIVE | F | 65 | ADCA | ACINAR and |
| | SMOKER | | | | | PAPILLARY |
| PATIENT2 | PREVIOUS | ALIVE | M | 62 | ADCA | SOLID |
| | SMOKER | | | | | |
| | <15 y | | | | | |
| PATIENT3 | NEVER | ALIVE | F | 63 | ADCA | ACINAR |
| | SMOKER | | | | | |
| PATIENT4 | SMOKER | ALIVE | M | 66 | SCC | |
| PATIENT5 | SMOKER | ALIVE | M | 68 | SCC | |
| TUMOR | | | | | | |
| INFILTRATING | | | | | | |
| TREG FROM | | | | | | |

| NSCLC | | | | | | |
|---|---|---|---|---|---|---|
| (SINGLE | | pTNM: | pTNM: | pTNM: | | CAPTURED |
| CELL qPCR | GRADE | T | N | M | STAGE | SINGLE CELLS |
| PATIENT1 | G2 | 2a | 0 | 0 | IB | 71 |
| PATIENT2 | G2 | 1b | 0 | 0 | IA | 61 |
| PATIENT3 | G1 | 1a | 0 | 0 | IA | 44 |
| PATIENT4 | G2 | 2a | 0 | 0 | IB | 55 |
| PATIENT5 | G3 | 1b | 0 | 0 | IA | 55 |

NSLC = Non Small Cell Lung Cancer
ADC = Adenocarcinoma
SCC = Squamous Cell Carcinoma
(T) = Tumor Sample
(H) = Healthy Tissue

| CRC | | | | | | | |
|---|---|---|---|---|---|---|---|
| PATIENTS | | | | | | | |
| LIST (RNA | | | | | | | |
| SEQUENCING) | (T) Th1 | (T) Th17 | (T) Treg | (H) Th1 | (H) Th17 | (H) Treg | GENDER |
| PATIENT1 | | | SQ_0389 | SQ_0386 | SQ_0387 | SQ_0388 | M |
| PATIENT2 | SQ_0427 | SQ_0434 | | | | SQ_0418 | F |
| PATIENT3 | SQ_0423 | SQ_0436 | SQ_0411 | | | | M |
| PATIENT4 | SQ_0426 | SQ_0437 | SQ_0413 | SQ_0428 | SQ_0439 | SQ_0417 | M |
| PATIENT5 | SQ_0425 | | SQ_0412 | SQ_0429 | SQ_0441 | SQ_0422 | M |
| PATIENT6 | SQ_0424 | | SQ_0415 | SQ_0431 | SQ_0442 | SQ_0421 | M |
| PATIENT7 | | SQ_0435 | SQ_0416 | SQ_0432 | SQ_0438 | SQ_0420 | F |
| PATIENT8 | | | SQ_0414 | | | | F |
| PATIENT9 | | SQ_0433 | | SQ_0430 | SQ_0440 | SQ_0419 | M |
| CRC | | | | | | | |
| PATIENTS | | HISTO- | | | | | |
| LIST (RNA | | TYPE | | pTNM: | pTNM: | pTNM: | |
| SEQUENCING) | AGE(y) | MAJOR | GRADE | T | N | M | STAGE |
| PATIENT1 | 76 | ADC | G2 | 3 | 1A | 0 | IIIB |
| PATIENT2 | 68 | ADC | G2 | 3 | 0 | 0 | IIA |
| PATIENT3 | 80 | ADC | G2 | 4B | 1B | 0 | IIIB |
| PATIENT4 | 79 | ADC | G2 | 3 | 1A | 0 | IIIB |
| PATIENT5 | 78 | ADC | G2 | 3 | 0 | 0 | IIA |
| PATIENT6 | 69 | MUC | — | 3 | 1B | 0 | IIIB |
| | | ADC | | | | | |
| PATIENT7 | 84 | ADC | G2 | 4B | 0 | 0 | IIC |
| PATIENT8 | 75 | MUC | — | 3 | 1C | 0 | IIIB |
| | | ADC | | | | | |
| PATIENT9 | 54 | ADC | G2 | 2 | 0 | 0 | I |
| ADC = Adenocarcinoma | | | | | | | |
| MUC ADC = Mucinous Adenocarcinoma | | | | | | | |
| CRIB ADC = Cribrous Adenocarcinoma | | | | | | | |
| (T) = Subsets purified from Tumor Sample | | | | | | | |
| (H) = Subsets purified from Healthy Tissue | | | | | | | |
| TUMOR | | | | | | | |
| INFILTRATING | | | | | | | |
| TREG FROM | | | HISTO- | | | | |
| CRC (SINGLE | | | TYPE | | pTNM: | | |
| CELL qPCR) | GENDER | AGE(y) | MAJOR | GRADE | T | | |
| PATIENT1 | M | 64 | ADC | 2 | 3 | | |
| PATIENT2 | M | 59 | CRIB | — | 3 | | |
| | | | ADC | | | | |
| PATIENT3 | F | 75 | MUC | — | 4A | | |
| | | | ADC | | | | |

| | | | | | |
|---|---|---|---|---|---|
| PATIENT4 | M | 71 | ADC | 1 | 3 |
| PATIENT5 | M | 64 | ADC | 2 | 3 |

TUMOR

| INFILTRATING | | | | |
|---|---|---|---|---|
| TREG FROM CRC | pTNM: | pTNM: | | CAPTURED |
| (SINGLE CELL qPCR) | N | M | STAGE | SINGLE CELLS |
| PATIENT1 | 0 | 0 | IIA | 62 |
| PATIENT2 | 0 | 0 | IIA | 66 |
| PATIENT3 | 2B | 0 | IIIC | 65 |
| PATIENT4 | 0 | 0 | IIA | 63 |
| PATIENT5 | 0 | 0 | IIA | 64 |

ADC = Adenocarcinoma
MUC ADC = Mucinous Adenocarcinoma
CRIB ADC = Cribrous Adenocarcinoma
(T) = Subsets purified from Tumor Sample
(H) = Subsets purified from Healthy Tissue

CRC: colorectal cancer;
NSCLC: non-small cell lung cancer;
(T): Tumor Sample;
(H): Healthy Tissue;
ADC: Adenocarcinoma;
SCC: Squamous Cell Carcinoma;
MUC ADC: Mucinous Adenocarcinoma.

To assess Treg cell function, inventors tested their suppressor activity and showed that Treg cells infiltrating either type of tumor tissues have a remarkably stronger suppressive activity in vitro compared to Treg cells isolated from the adjacent normal tissue and peripheral blood of the same patients (FIG. 1B).

The polyadenylated RNA fraction extracted from the sorted CD4+ Treg, Th1, and Th17 cells was then analyzed by pair-end RNA sequencing obtaining about 4 billion mapped “reads” (Table 1). First, inventors interrogated RNA-sequencing data of CD4+ T cells infiltrating both CRC and NSCLC and their matched normal tissues, to quantitate mRNA expression of known immune checkpoints and their ligands. Second, inventors analyzed RNA-seq data of CRC and NSCLC, as well as of normal colon and lung samples.

Inventors found that several immune checkpoints and their ligands transcripts were strikingly upregulated in tumor infiltrating Treg cells compared to both normal tissue and peripheral blood-derived Treg cells, as well as to T and B lymphocyte subsets purified from peripheral blood mononuclear cells (PBMCs) (FIGS. 10C and 60 and Table Ill).

| | Treg_Tumor_ | Treg_Tumor_ | Treg_Tissue_ | Treg_Tissue_ | Treg healthy |
|---|---|---|---|---|---|
| GENE | Infiltrating | Infiltrating | Inflitrating | Inflitrating | Peripheral |
| NAME | CRC | NSCLC | Colon | Lung | Blood |
| ADORA2A | 14.69 | 24.06 | 17.97 | 44.84 | 18.52 |
| BTLA | 554.04 | 742.11 | 389.51 | 208.76 | 108.2 |
| BTNL2 | 0 | 0.14 | 0.29 | 0 | 0.75 |
| (BTLN2) | | | | | |
| C10orf54 | 779.38 | 872.36 | 555.47 | 1405.63 | 1111.37 |
| (VISTA) | | | | | |
| CD160 | 58.39 | 38.24 | 51.87 | 34.54 | 36.55 |
| CD200 | 268.39 | 283.21 | 282.05 | 104.64 | 99.59 |
| CD200R1 | 95.89 | 136.08 | 81.36 | 349.99 | 59.03 |
| CD244 | 34.46 | 31.21 | 29.59 | 128.35 | 47.8 |
| CD27 | 710.13 | 1068.55 | 583.58 | 496.38 | 468.93 |
| CD274 | 1050.94 | 645.66 | 576.59 | 390.71 | 120.19 |
| (PD-L1) | | | | | |
| CD276 | 16.85 | 72.3 | 10.44 | 65.98 | 3.61 |
| CD28 | 4770.41 | 4585.17 | 5446.29 | 3687.01 | 5179.32 |
| CD40 | 112.04 | 161.29 | 80.64 | 93.3 | 34.71 |
| CD40LG | 135.51 | 143.07 | 360.09 | 418.55 | 104.22 |
| CD44 | 13049.36 | 8518.98 | 13513.69 | 19851 | 16013.71 |
| CD48 | 346.61 | 489.78 | 494.58 | 594.83 | 1523.63 |
| CD70 | 426.35 | 269.38 | 318.97 | 249.48 | 101.67 |
| CD80 | 632.12 | 483.34 | 318.48 | 269.06 | 114.41 |
| CD86 | 29.52 | 78.86 | 52.72 | 278.86 | 3.87 |
| CTLA4 | 6798.82 | 10378.3 | 4810.74 | 5340.06 | 4806.23 |
| HAVCR2 | 577.57 | 633.27 | 265.84 | 487.62 | 49.81 |
| (TIM-3) | | | | | |
| HHLA2 | 3.41 | 3.66 | 4.47 | 9.28 | 12.7 |
| ICOS | 6830.94 | 7339.08 | 4119.2 | 5211.71 | 3398.28 |
| ICOSLG | 58.02 | 8.86 | 59.13 | 33.5 | 76.5 |
| (B7RP1) | | | | | |
| IDO1 | 3.86 | 83.81 | 9.51 | 5.15 | 2.36 |
| IDO2 | 0.22 | 2.25 | 1.41 | 5.15 | 1.58 |
| KIR3DL1 | 0.38 | 0.43 | 0.28 | 4.64 | 0.9 |
| (KIR) | | | | | |
| LAG3 | 705.14 | 1956.22 | 2181.52 | 1505.63 | 127.02 |
| LAIR1 | 277.06 | 194.09 | 551.94 | 874.72 | 346.22 |
| LGALS9 | 1175.81 | 1530.47 | 1160.89 | 1593.26 | 592.56 |
| (Galectin-9) | | | | | |
| NRP1 | 7.38 | 36.24 | 8.89 | 106.7 | 8.59 |
| PDCD1LG2 | 214.51 | 223.04 | 61.89 | 25.77 | 12.12 |
| (PD-L2) | | | | | |
| PDCD1 | 467.22 | 496.56 | 405.01 | 676.27 | 111.26 |
| (PD1) | | | | | |
| TIGIT | 14821.45 | 14747.79 | 10986.74 | 4901.41 | 4611.14 |
| TMIGD2 | 28.38 | 16.64 | 78.3 | 75.77 | 71.27 |
| TNFRSF14 | 2230.85 | 2677.32 | 2297.43 | 2675.7 | 2274.82 |
| (HVEM) | | | | | |
| TNFRSF18 | 4038.86 | 4078.14 | 2871.78 | 3071.57 | 333.36 |
| (GITR) | | | | | |
| TNFRSF25 | 5236.86 | 4188.61 | 4986.56 | 5111.71 | 3587.58 |
| TNFRSF4 | 4222.16 | 4642.56 | 2873.16 | 2992.18 | 400.56 |
| (OX40) | | | | | |
| TNFRSF8 | 155.59 | 430.23 | 115.57 | 208.24 | 30.89 |
| (CD30) | | | | | |
| TNFRSF9 | 2921.72 | 3128.82 | 898.69 | 1739.13 | 502.86 |
| (4-1BB) | | | | | |
| TNFSF14 | 148.57 | 183.77 | 223.49 | 421.12 | 105.12 |
| (LIGHT) | | | | | |
| TNFSF15 | 1.58 | 3.75 | 0.89 | 25.77 | 1.23 |
| TNFSF18 | 0.4 | 1.11 | 0.53 | 0 | 0.45 |
| TNFSF4 | 110.82 | 136.82 | 100.95 | 98.97 | 16.33 |
| (OX40LG) | | | | | |
| TNFSF9 | 26.79 | 19.48 | 19.72 | 29.9 | 7.41 |
| (CD137L) | | | | | |
| VTCN1 | 1.12 | 4.49 | 1.48 | 1.55 | 2.65 |
| (B7-H4) | | | | | |

RNA-seq normalized counts data for selected immune checkpoints genes and their ligands in all the subsets analyzed.

These findings highlight the specific expression patterns of immune checkpoints and their ligands in tumor infiltrating Treg and effector cells and suggest that their functional relevance should be investigated directly at tumor sites.

### Tumor-Infiltrating Treg Cells Express a Specific Gene Signature

The inventors then asked whether tumor infiltrating Treg cells could be defined by specific gene expression patterns.

To identify signature transcripts of tumor-infiltrating Treg cells, the inventors included in the expression pattern analyses the transcriptome dataset they previously obtained from different T and B lymphocyte subsets purified from PBMCs (Ranzani et al., 2015). In so doing, the inventors obtained a signature of 328 transcripts whose expression is higher in tumor infiltrating Treg cells (Wilcoxon Mann Whitney test p<2.2×10−16) (FIG. 2, and Table IV compared to the other lymphocyte subsets purified from non-tumoral tissues and from PBMCs of healthy or neoplastic patients.

| | Treg_Tumor_ | Treg_Tumor_ | Treg_Tissue_ | Treg_Tissue_ | Treg healthy |
|---|---|---|---|---|---|
| Gene | Infiltrating | Infiltrating | Inflitrating | Inflitrating | Peripheral |
| Name | CRC | NSCLC | Colon | Lung | Blood |
| AC019206.1 | 15.41 | 8.72 | 12.89 | 12.04 | 29.46 |
| ACAA2 | 305.76 | 499.02 | 497.41 | 526.58 | 614.28 |
| ACOT9 | 918.3 | 803.71 | 1361.82 | 2180.66 | 1272.07 |
| ACOX3 | 183.48 | 384.73 | 469.06 | 506.97 | 439.27 |
| ACP5 | 267.7 | 837.72 | 859.77 | 1872.29 | 1483.27 |
| ACSL4 | 1154.87 | 1384.88 | 1903.56 | 2170.94 | 2043.91 |
| ACTA2 | 86.65 | 270.74 | 108.76 | 234.86 | 232.15 |
| ACTG2 | 10.69 | 6.16 | 22.68 | 21.11 | 36.14 |
| ADAM10 | 2378.26 | 3051.7 | 2545.29 | 3600.38 | 3167.56 |
| ADAT2 | 927.45 | 1272.17 | 1214.4 | 2094.25 | 3103.21 |
| ADPRH | 136.34 | 460.61 | 352.57 | 836.7 | 718.74 |
| AHCYL1 | 914.19 | 1271.5 | 1269.55 | 1835.94 | 1711.94 |
| AHCYL2 | 305.15 | 570.67 | 525.24 | 790.1 | 856.25 |
| AKAP5 | 174.24 | 264 | 358.75 | 709.28 | 535.97 |
| AKIP1 | 261.47 | 273.85 | 225.25 | 436.84 | 360.48 |
| ANKRD10 | 2251.92 | 3433.73 | 2805.08 | 4192.8 | 4672.81 |
| ARHGEF12 | 1371.05 | 2064.05 | 1536.04 | 3069.77 | 2637.79 |
| ARHGEF4 | 19.42 | 71.47 | 28.87 | 195.02 | 252.84 |
| ARL6IP5 | 3008.69 | 4385.74 | 4051.43 | 4983.16 | 4712.48 |
| ARNTL2 | 20.4 | 201.3 | 281.95 | 560.77 | 445.13 |
| ATP13A3 | 3776.14 | 4020.7 | 4688.02 | 6688.94 | 6967.94 |
| ATP2C1 | 1491.87 | 1399.81 | 1553.57 | 2029.41 | 1819.78 |
| AURKA | 24.56 | 50.12 | 79.89 | 66.37 | 87.07 |
| BATF | 820.97 | 3325.93 | 1698.92 | 5052.64 | 2727.65 |
| BCL2L1 | 212.64 | 478.8 | 537.61 | 554.11 | 892.28 |
| BIRC5 | 14.74 | 20.27 | 20.62 | 25.03 | 44.99 |
| C17orf96 | 19 | 174.31 | 159.79 | 239.88 | 377.03 |
| C5orf63 | 146.45 | 201.44 | 112.88 | 228.2 | 357.09 |
| CABLES1 | 59.04 | 196.68 | 125.77 | 473.94 | 386.73 |
| CACNB2 | 67.43 | 50.49 | 40.21 | 169.83 | 105.62 |
| CADM1 | 113.76 | 602.72 | 115.46 | 1766.12 | 901.32 |
| CALM3 | 2474.48 | 2829.3 | 2675.18 | 2954.03 | 4107.03 |
| CARD16 | 370.31 | 696.36 | 493.29 | 1220.7 | 823.89 |
| CARD17 | 41.87 | 96.94 | 54.12 | 101.19 | 132.95 |
| CASP1 | 925.29 | 1453.84 | 1521.09 | 2028.95 | 1980.45 |
| CASQ1 | 52.11 | 31.21 | 24.74 | 135.08 | 174.95 |
| CCNB2 | 18.28 | 27.62 | 34.02 | 51.57 | 58.08 |
| CCR8 | 255.66 | 578.27 | 1355.63 | 3127.33 | 2069.11 |
| CD177 | 2.36 | 204.74 | 299.99 | 718.58 | 470.27 |
| CD27 | 468.93 | 583.58 | 496.38 | 710.13 | 1068.55 |
| CD274 | 120.19 | 576.59 | 390.71 | 1050.94 | 645.66 |
| CD7 | 1622.12 | 6900.01 | 2829.82 | 9053.96 | 6919.59 |
| CDCA2 | 19.24 | 35.09 | 49.48 | 68.21 | 49.95 |
| CDH24 | 57.67 | 57.11 | 89.69 | 148.93 | 105.02 |
| CDK6 | 602.97 | 2175.36 | 2463.85 | 3580.4 | 3238.58 |
| CEACAM1 | 360.01 | 340.84 | 326.28 | 381.79 | 732.86 |
| CENPM | 43,72 | 39.12 | 61.85 | 72.94 | 61.32 |
| CEP55 | 56.18 | 88.17 | 223.71 | 220.17 | 273.64 |
| CGA | 1.08 | 13.59 | 22.68 | 334.28 | 9.73 |
| CHRNA6 | 14.46 | 218.49 | 67.52 | 336.38 | 504.28 |
| CHST11 | 1822.7 | 2085.92 | 2806.11 | 2790.19 | 2535.23 |
| CHST2 | 75.46 | 218.75 | 156.7 | 458.24 | 604.97 |
| CHST7 | 141.3 | 341.87 | 426.79 | 1087.21 | 333.3 |
| CIT | 89.25 | 105.13 | 155.15 | 150.2 | 262.67 |
| CLNK | 153.06 | 288.36 | 248.96 | 340.12 | 528.54 |
| CNIH1 | 1028.31 | 1005.46 | 935.03 | 2336.95 | 1101.87 |
| COL9A2 | 149.87 | 278.77 | 357.72 | 889.47 | 805.72 |
| CORO1B | 481.34 | 667.37 | 861.83 | 774.65 | 1040.47 |
| COX10 | 305.31 | 399.33 | 397.93 | 447.17 | 612.29 |
| CRADD | 77.04 | 155.66 | 277.31 | 394.31 | 306.61 |
| CREB3L2 | 739.04 | 1289.66 | 1415.94 | 2984.54 | 2590.37 |
| CSF1 | 313.09 | 1629.13 | 1609.75 | 2204.79 | 3288.67 |
| CSF2RB | 1069.75 | 1275.49 | 1290.69 | 2036.76 | 2531.99 |
| CTLA4 | 4806.23 | 4810.74 | 5340.06 | 6798.82 | 10378.3 |
| CTSC | 1026.76 | 2196.93 | 2514.88 | 3030.74 | 2767.27 |
| CTTNBP2NL | 85 | 200.53 | 248.45 | 500.75 | 267.16 |
| CX3CR1 | 9.57 | 63.99 | 123.71 | 341.79 | 293.28 |
| CXCL13 | 1.07 | 255.23 | 1145.33 | 1270.98 | 11433.26 |
| CYB5B | 714.26 | 1129.39 | 947.4 | 1156.4 | 1221.22 |
| CYP7B1 | 9.83 | 210.33 | 29.38 | 186.99 | 161.17 |
| DCPS | 153.25 | 210.26 | 210.82 | 191.31 | 271.71 |
| DFNB31 | 561.87 | 1636.56 | 1727.79 | 4251.83 | 2526.15 |
| DIRAS3 | 1.9 | 4.59 | 3.61 | 26.01 | 35.64 |
| DLGAP5 | 7.89 | 14.46 | 20.62 | 27.41 | 49.7 |
| DNPH1 | 160.15 | 650.05 | 321.13 | 683.55 | 576.77 |
| DOC2B | 10.47 | 3.42 | 5.15 | 14.23 | 238.86 |
| DPYSL2 | 208.98 | 189.08 | 580.4 | 591.32 | 618.42 |
| EBI3 | 7.47 | 103.59 | 56.7 | 148.96 | 200.74 |
| ECEL1 | 3.7 | 150.7 | 34.02 | 199.17 | 794.51 |
| EGLN1 | 977.29 | 969.32 | 1021.11 | 1381.2 | 1271.06 |
| EML2 | 861.51 | 1601.25 | 1643.25 | 2156.04 | 1957.43 |
| ENTPD1 | 752.88 | 2078.17 | 1447.38 | 4321.79 | 4162.57 |
| ERI1 | 354.33 | 862.86 | 932.45 | 1200.06 | 1070.15 |
| ETFA | 414.08 | 586.15 | 534.01 | 615.35 | 689.14 |
| ETV7 | 93.62 | 511.26 | 361.85 | 728.85 | 1111.55 |
| EVA1B | 21.39 | 35.63 | 26.8 | 42.86 | 47.36 |
| F5 | 2343.39 | 2346.94 | 2499.41 | 4868.41 | 4729.97 |
| FAAH2 | 244.19 | 431.76 | 209.27 | 737.44 | 699.42 |
| FAIM2 | 15.05 | 33.47 | 57.21 | 69.26 | 117.28 |
| FAM184A | 192.41 | 742.47 | 525.24 | 706.33 | 891.02 |
| FAM19A2 | 311.38 | 204.56 | 302.57 | 264.46 | 748.09 |
| FAM98B | 314.26 | 664.69 | 491.22 | 698.92 | 657.42 |
| FAS | 2337.14 | 5167.46 | 2712.81 | 5982.39 | 3656.21 |
| FBXO45 | 460.56 | 783.06 | 631.43 | 964.13 | 894.23 |
| FCRL3 | 1161.64 | 1997.02 | 938.63 | 3281.36 | 2699.01 |
| FKBP1A | 733.83 | 1240.62 | 1174.19 | 1377.67 | 1578.09 |
| FLNB | 1671.04 | 1363.04 | 1394.81 | 3395.38 | 2307.44 |
| FLVCR2 | 69.84 | 579.55 | 388.13 | 744.8 | 528.01 |
| FNDC3B | 377.47 | 501.27 | 506.17 | 1111.07 | 531.12 |
| FOXA1 | 2.7 | 11.87 | 17.01 | 70.68 | 18.22 |
| FOXM1 | 56.39 | 74.94 | 108.24 | 88.16 | 125.31 |
| FOXP3 | 6586.98 | 10713.12 | 6060.66 | 13483.77 | 11472.41 |
| FUCA2 | 107.56 | 175.46 | 160.82 | 249.54 | 315.45 |
| GADD45A | 745.14 | 1431.9 | 884.51 | 3681.24 | 1396.98 |
| GCNT1 | 99.22 | 632.16 | 608.75 | 1133.62 | 845.83 |
| GK | 637.31 | 1994.73 | 2430.34 | 5200.55 | 2065.35 |
| GLB1 | 563.96 | 819.22 | 873.17 | 1077.84 | 854.94 |
| GLCCI1 | 1557.57 | 3211.73 | 1753.04 | 3189.77 | 2909.06 |
| GLDC | 19.25 | 20.56 | 25.26 | 31.21 | 74.61 |
| GLRX | 1213.06 | 1251.64 | 1512.85 | 1764.61 | 1872 |
| GNG4 | 5.08 | 79.18 | 64.43 | 197.1 | 343.93 |
| GNG8 | 11.94 | 63.28 | 10.82 | 67.63 | 175.16 |
| GRSF1 | 1277.4 | 1725.67 | 1397.9 | 2899.76 | 2343.4 |
| GSK3B | 1099.5 | 1267.18 | 1208.73 | 1333.16 | 1454.67 |
| GTF3C6 | 313.17 | 579.04 | 445.86 | 617.48 | 597.55 |
| GTSF1L | 13.67 | 20.36 | 15.46 | 44.6 | 99.03 |
| HADHB | 1179.61 | 1207.14 | 1287.59 | 1396.89 | 1521.16 |
| HAP1 | 92.39 | 180.51 | 74.22 | 292.97 | 577 |
| HAVCR2 | 49.81 | 265.84 | 487.62 | 577.57 | 633.27 |
| HECW2 | 17.63 | 98.93 | 38.66 | 111.21 | 177.5 |
| HIBCH | 124.32 | 290.04 | 226.8 | 348.34 | 332.88 |
| HIVEP3 | 358.34 | 649.68 | 893.27 | 1091.96 | 1316.89 |
| HJURP | 8.55 | 18.52 | 15.98 | 27.13 | 39.99 |
| HOXA1 | 16.66 | 15.22 | 14.95 | 25.57 | 44.75 |
| HPRT1 | 442.58 | 532.66 | 542.25 | 811.75 | 724.15 |
| HPSE | 248.88 | 676.54 | 515.45 | 674.09 | 754.04 |
| HS3ST3B1 | 1222.43 | 1930.88 | 1980.87 | 2609.49 | 2431.83 |
| HSDL2 | 242.56 | 611.72 | 285.56 | 785.27 | 921.97 |
| HTATIP2 | 567.61 | 1439.29 | 997.4 | 3285.86 | 1576.24 |
| ICA1 | 94.65 | 371.57 | 113.91 | 487.68 | 411.64 |
| ICOS | 3398.28 | 4119.2 | 5211.71 | 6830.94 | 7339.08 |
| IGFLR1 | 67.43 | 78.13 | 92.78 | 108.12 | 185.13 |
| IKZF2 | 6061.48 | 6317.6 | 4919.45 | 9983.52 | 8551.49 |
| IKZF4 | 1422.66 | 2362.49 | 1258.21 | 3745.25 | 3958.19 |
| IL12RB2 | 120.8 | 369.84 | 509.78 | 835.92 | 877.51 |
| IL17REL | 9.74 | 23.21 | 34.02 | 52.62 | 57.04 |
| IL1R1 | 506.51 | 9670.81 | 2766.42 | 7852.18 | 5585.89 |
| IL1R2 | 41.72 | 1225.4 | 526.79 | 2117.34 | 1793.21 |
| IL1RL1 | 17.37 | 135.26 | 44.33 | 715.42 | 71.67 |
| IL1RL2 | 8.65 | 76.53 | 28.35 | 74.81 | 59.47 |
| IL21R | 708.61 | 1355.83 | 1715.93 | 3092.3 | 3514.36 |
| IL2RA | 5244.31 | 9685.38 | 5627.68 | 11454.42 | 12731.31 |
| IL2RB | 6716.4 | 14249.6 | 12502.75 | 17733 | 18564.35 |
| IL32 | 4332.08 | 13202.73 | 9755.92 | 11766.98 | 13883.45 |
| IL7 | 117.66 | 230.78 | 165.97 | 257.71 | 178.1 |
| INPP1 | 124.25 | 497.01 | 312.88 | 458.2 | 487.93 |
| INPP5F | 787.92 | 2172.55 | 830.9 | 2189.48 | 1549.46 |
| ISOC1 | 233.44 | 329.49 | 400.5 | 514.43 | 335.93 |
| ITFG1 | 313.34 | 324.11 | 402.05 | 396.94 | 511.86 |
| JAK1 | 10779.78 | 11919.66 | 10072.4 | 17755.9 | 11521.32 |
| JAKMIP1 | 291.14 | 387.49 | 1063.89 | 756.36 | 953.47 |
| KAT2B | 3145.05 | 3910.01 | 4756.57 | 5520.88 | 4632.76 |
| KIF14 | 20.18 | 25.43 | 31.96 | 36.73 | 59.61 |
| KIF15 | 20.64 | 29.67 | 51.03 | 41.9 | 68.63 |
| KIF20A | 9.84 | 14.93 | 7.22 | 20.97 | 32.72 |
| KLHDC7B | 131.39 | 211.42 | 188.65 | 245.3 | 394.73 |
| KSR1 | 837.87 | 1569.86 | 1176.77 | 2241.36 | 1847.72 |
| LAPTM4B | 86.42 | 369.78 | 181.44 | 938.88 | 738.38 |
| LAX1 | 1135.24 | 1155.91 | 1406.15 | 1721.7 | 1854.78 |
| LAYN | 441.73 | 796.76 | 859.25 | 2650.24 | 1681.25 |
| LEPR | 58.77 | 130.22 | 129.38 | 137.47 | 237.88 |
| LEPROT | 614.73 | 860.55 | 676.79 | 1044.66 | 1296.13 |
| LHFP | 1.58 | 10.38 | 9.79 | 18.09 | 63.16 |
| LIMA1 | 404.55 | 727.57 | 1017.5 | 1064.46 | 1570.15 |
| LMCD1 | 115.76 | 104.74 | 112.37 | 257.92 | 404.7 |
| LOC388813 | 7.42 | 45.99 | 28.87 | 86.3 | 60.63 |
| LRG1 | 17.67 | 61.54 | 46.39 | 71.6 | 78.3 |
| LRRC61 | 98.78 | 291.45 | 138.66 | 292.51 | 314.79 |
| LTA | 214.07 | 516.57 | 270.61 | 351.26 | 747.01 |
| LXN | 67.37 | 91.06 | 75.77 | 114.23 | 133.43 |
| LY75 | 249.92 | 970.85 | 680.91 | 1302.79 | 1624.82 |
| MAGEH1 | 461.13 | 1349.51 | 448.96 | 2800.36 | 3719.29 |
| MALT1 | 3362.14 | 3568.46 | 2743.74 | 5892.86 | 4776.24 |
| MAP1LC3A | 70,92 | 110,44 | 119,07 | 272,07 | 169,3 |
| MAP3K5 | 1865.12 | 2189.99 | 1787.06 | 2822.55 | 2265.54 |
| MAST4 | 1053.08 | 2239.36 | 2198.39 | 3373.36 | 1855.42 |
| MAT2B | 2305.62 | 4050.5 | 2959.2 | 4435.41 | 4159.25 |
| MCCC2 | 737.75 | 875.78 | 873.69 | 1018.1 | 1245.79 |
| MELK | 28.77 | 50.08 | 83.5 | 72.28 | 83.06 |
| METTL7A | 280.99 | 442.99 | 385.04 | 845.09 | 1671.74 |
| METTL8 | 318.99 | 882.21 | 377.82 | 880.99 | 1413.12 |
| MGME1 | 236.76 | 332.08 | 342.77 | 400.19 | 552.69 |
| MGST2 | 54.22 | 87.18 | 69.59 | 147.04 | 148.13 |
| MICAL2 | 354.6 | 1601.79 | 1813.35 | 1910.22 | 3188.92 |
| MINPP1 | 85.19 | 204.32 | 211.85 | 243.22 | 290.02 |
| MKI67 | 192.68 | 206.77 | 518.03 | 372.61 | 650.04 |
| MREG | 120.75 | 119.91 | 226.28 | 229.41 | 325.33 |
| MYL6B | 122.13 | 182.71 | 107.73 | 174.22 | 252.52 |
| MYO5C | 95.68 | 122.36 | 157.21 | 130.81 | 347.49 |
| NAB1 | 508.21 | 973.74 | 1261.31 | 1831.77 | 1227.51 |
| NCALD | 111.73 | 163.32 | 272.67 | 283.43 | 370.26 |
| NCAM1 | 7.88 | 58.27 | 39.69 | 207.45 | 213.23 |
| NCF4 | 509.63 | 630.55 | 880.39 | 894.67 | 1176.84 |
| NCOA1 | 2088.38 | 2062.57 | 1941.7 | 2367.54 | 2618.11 |
| NDFIP2 | 77.99 | 529.73 | 618.54 | 829.53 | 987.25 |
| NEMP2 | 382.56 | 478.4 | 475.76 | 565.18 | 634.41 |
| NETO2 | 145.84 | 559.95 | 773.69 | 1490.82 | 1137.73 |
| NEURL3 | 4.04 | 29.74 | 12.37 | 24.02 | 35.49 |
| NFAT5 | 2075.17 | 3880.92 | 3923.6 | 4786.04 | 5295.06 |
| NFE2L3 | 279.28 | 590.19 | 560.29 | 743.24 | 1114.26 |
| NFYC | 588.49 | 713.51 | 756.16 | 733.52 | 798.27 |
| NHS | 7.27 | 18.73 | 55.15 | 60.16 | 159.44 |
| NPTN | 525.86 | 838.02 | 897.91 | 1007.87 | 969.1 |
| NTNG2 | 117.04 | 296.81 | 534.52 | 669.43 | 1001.58 |
| NTRK1 | 20.85 | 27.9 | 155.15 | 88.29 | 161.78 |
| NUSAP1 | 199.28 | 266.11 | 445.86 | 635.51 | 365.17 |
| NXT2 | 221.6 | 263.39 | 226.8 | 285.15 | 302.01 |
| OSBP2 | 111.03 | 89.82 | 127.83 | 195.47 | 244.93 |
| PAK2 | 4621.62 | 6173.86 | 5024.6 | 7194.78 | 6376.28 |
| PAM | 582.52 | 904.05 | 1069.56 | 1365.03 | 1631.64 |
| PANX2 | 3.7 | 76.02 | 15.46 | 97.12 | 71.72 |
| PAQR4 | 16.99 | 46.54 | 62.37 | 92.6 | 65.27 |
| PARD6G | 55.86 | 172.18 | 249.99 | 546.52 | 182.4 |
| PARK7 | 1271.06 | 1563.96 | 1283.47 | 1764.8 | 1764.91 |
| PCTP | 49.2 | 173.47 | 163.4 | 253.27 | 270.62 |
| PDCD1LG2 | 12.12 | 61.89 | 25.77 | 214.51 | 223.04 |
| PDGFA | 6.19 | 38.74 | 159.79 | 154.17 | 153.03 |
| PEX3 | 179.31 | 239.78 | 205.66 | 326.61 | 291.17 |
| PGM2 | 316.91 | 419.51 | 454.63 | 471.89 | 487.85 |
| PHKA1 | 8.59 | 19.98 | 28.87 | 107.79 | 109.7 |
| PIGU | 147.54 | 205.18 | 184.53 | 220.25 | 265.12 |
| PLA2G4C | 22.16 | 128.81 | 65.98 | 245.65 | 159.6 |
| PPM1G | 1974.96 | 2324.16 | 2563.85 | 2751.69 | 2598.5 |
| PRDX3 | 466.56 | 854.12 | 745.34 | 890.58 | 1052.67 |
| PRKCDBP | 4.45 | 6.8 | 19.07 | 28.51 | 27.92 |
| PROB1 | 53.7 | 140.39 | 109.79 | 177.19 | 272.89 |
| PTGIR | 96.17 | 147.61 | 107.21 | 214.61 | 449.25 |
| PTP4A3 | 134.06 | 262.63 | 463.39 | 340.08 | 667.84 |
| PTPRJ | 2654.92 | 3999.84 | 5584.38 | 6101.63 | 7239.3 |
| PTTG1 | 211.97 | 198.56 | 236.59 | 302.53 | 335.68 |
| RAB15 | 160.6 | 470.25 | 302.05 | 420.06 | 519.4 |
| RAD51AP1 | 29.89 | 46.33 | 40.21 | 49.23 | 51.73 |
| RASAL1 | 18.87 | 53.37 | 50 | 87.38 | 238.78 |
| RBKS | 67.62 | 56.45 | 133.5 | 141.16 | 85.46 |
| RCBTB1 | 1154.33 | 1312.01 | 1131.41 | 1960.76 | 1384.84 |
| RDH10 | 194.04 | 311.58 | 467.51 | 658.5 | 1448.57 |
| REXO2 | 487.9 | 832.35 | 648.44 | 852.58 | 987.43 |
| RFK | 378.31 | 396.91 | 292.26 | 460.78 | 452.8 |
| RGS1 | 16547.6 | 15176.27 | 18057.75 | 23425.18 | 17168.17 |
| RHOC | 78.07 | 230.17 | 207.21 | 317.85 | 290.86 |
| RMI2 | 19.46 | 76.58 | 39.69 | 70.44 | 73.47 |
| RNF145 | 1625.11 | 3074.78 | 2117.47 | 4417.29 | 3266.94 |
| RNF207 | 41.75 | 469.3 | 314.94 | 723.56 | 765.87 |
| RRAGB | 281.49 | 274.98 | 196.9 | 384.81 | 506.1 |
| RYBP | 1861.27 | 2273.72 | 2496.32 | 3178.31 | 2818.02 |
| SEC14L6 | 6.42 | 86.23 | 27.32 | 179.47 | 274.97 |
| SEC24A | 718 | 917.25 | 1157.7 | 1259.04 | 1062.95 |
| SECTM1 | 69.01 | 1347.35 | 725.75 | 2354.1 | 1511.04 |
| SEPT3 | 15.6 | 59.23 | 49.48 | 149.11 | 244.4 |
| SGPP2 | 428.14 | 656.73 | 364.94 | 1001.71 | 809.92 |
| SH3RF2 | 20.9 | 18.3 | 65.98 | 98.4 | 196.34 |
| SIRPG | 433.99 | 605.49 | 317 | 575.41 | 1245.12 |
| SLC16A1 | 947.47 | 1385.08 | 1532.43 | 2050.74 | 1460.73 |
| SLC25A12 | 246.72 | 323.6 | 423.18 | 406.15 | 498.91 |
| SLC35E3 | 385.3 | 451.16 | 370.09 | 582.86 | 653.13 |
| SLC35F2 | 378.22 | 795.55 | 688.64 | 1130.81 | 880.5 |
| SLC41A1 | 1194.29 | 1119.86 | 1164.92 | 1401.41 | 1630.88 |
| SLC41A2 | 13.45 | 356.73 | 114.95 | 482.48 | 395.27 |
| SMAD1 | 15.34 | 53.93 | 30.41 | 63.54 | 87.46 |
| SMS | 565.6 | 760.65 | 719.57 | 818.12 | 735.99 |
| SNAP47 | 310.71 | 503.77 | 577.82 | 690.31 | 696.18 |
| SOCS2 | 245.77 | 405.76 | 463.39 | 605.25 | 611.78 |
| SOX4 | 128.76 | 244.57 | 218.04 | 1205.78 | 715.01 |
| SPATA24 | 38.86 | 77.02 | 36.6 | 66.43 | 94.41 |
| SPATC1 | 7.97 | 10.96 | 19.59 | 61.51 | 55.84 |
| SPATS2L | 366.98 | 891.61 | 1172.13 | 1430.11 | 1531.61 |
| SSH1 | 1890.01 | 3432.55 | 2771.06 | 4390.36 | 4552.26 |
| SSTR3 | 230.28 | 248.12 | 341.74 | 240.77 | 901.25 |
| STAC | 11.63 | 48.36 | 39.69 | 75.94 | 71.4 |
| STARD7 | 2415.01 | 3185.95 | 3024.66 | 3809.46 | 3445.47 |
| STRIP2 | 103.39 | 1002.96 | 540.19 | 716.49 | 1192.77 |
| SYT11 | 1078.51 | 1733.37 | 2080.36 | 2110.18 | 2818.39 |
| TADA3 | 677.14 | 893.74 | 852.04 | 880.43 | 1189.01 |
| TBC1D8 | 53.89 | 374.1 | 265.97 | 817.36 | 1087.39 |
| TDRD3 | 461.34 | 383.25 | 520.09 | 584.64 | 643.84 |
| TFRC | 3608.04 | 4612.18 | 5640.05 | 8107.35 | 10082.21 |
| THADA | 1102.51 | 1505.13 | 1467.48 | 3472.21 | 3171.99 |
| TIGIT | 4611.14 | 10986.74 | 4901.41 | 14821.45 | 14747.79 |
| TM9SF2 | 2048.03 | 2689.14 | 2665.91 | 2935.98 | 3358.4 |
| TMA16 | 172.88 | 180.92 | 137.11 | 304.24 | 192.53 |
| TMEM140 | 273.98 | 640.28 | 574.73 | 917.16 | 691 |
| TMEM184C | 520.19 | 508.83 | 599.98 | 1170.37 | 519.43 |
| TMOD1 | 14.75 | 72.22 | 32.47 | 150.93 | 89.62 |
| TMPRSS3 | 70.84 | 352.78 | 321.64 | 540.8 | 1106.85 |
| TMPRSS6 | 113.53 | 548.87 | 265.97 | 698.41 | 985.34 |
| TNFRSF18 | 333.36 | 2871.78 | 3071.57 | 4038.86 | 4078.14 |
| TNFRSF4 | 400.56 | 2873.16 | 2992.18 | 4222.16 | 4642.56 |
| TNFRSF8 | 30.89 | 115.57 | 208.24 | 155.59 | 430.23 |
| TNFRSF9 | 502.86 | 898.69 | 1739.13 | 2921.72 | 3128.82 |
| TNIP3 | 28.73 | 485.83 | 213.91 | 324.53 | 419.8 |
| TOR4A | 141.27 | 291.3 | 346.9 | 358.98 | 326.51 |
| TOX2 | 237.46 | 860.48 | 490.71 | 861.08 | 1264.13 |
| TP73 | 7.86 | 31.27 | 39.69 | 78.27 | 93.99 |
| TPMT | 357.13 | 354.93 | 305.66 | 480.15 | 519.82 |
| TPP1 | 2589.92 | 6024.92 | 4380.81 | 7164.96 | 6236.83 |
| TPX2 | 106.25 | 89.08 | 184.02 | 150.35 | 202.77 |
| TRAF3 | 1140.85 | 3231.25 | 2706.11 | 4078.84 | 3554.01 |
| TRIB1 | 927.27 | 1820.64 | 1482.95 | 2402.58 | 1469.85 |
| TRIM16 | 160.05 | 115.2 | 121.13 | 240.55 | 210.13 |
| TSPAN17 | 709.59 | 1721.26 | 1322.64 | 1685.38 | 1865.69 |
| TSPAN5 | 372.4 | 1167.46 | 723.69 | 1230.67 | 1398.7 |
| TST | 3.8 | 26.32 | 26.8 | 39.78 | 41.65 |
| TTBK1 | 13.41 | 164.27 | 99.48 | 380.69 | 460.64 |
| TTC22 | 237.9 | 386.91 | 323.19 | 483.96 | 451.61 |
| TWIST1 | 4.21 | 94.46 | 21.65 | 95.32 | 195.78 |
| UGP2 | 1950.41 | 3283.79 | 2562.82 | 3399.18 | 2864.71 |
| USP51 | 48.1 | 133.95 | 28.87 | 233.48 | 291.46 |
| UXS1 | 1661.1 | 2156.16 | 1600.47 | 2614.66 | 1914.74 |
| VANGL1 | 97.19 | 192.58 | 248.96 | 263.46 | 289.05 |
| VDR | 123 | 992.41 | 1771.6 | 2616.68 | 3656.18 |
| VWA5A | 426.29 | 550.67 | 373.7 | 604.53 | 739.57 |
| WDHD1 | 101.74 | 126.37 | 140.2 | 136.76 | 193.58 |
| WDTC1 | 1220.3 | 3855.35 | 2029.33 | 4398.54 | 3774.61 |
| WSB1 | 2837.49 | 3876.77 | 4697.29 | 5090.18 | 5383.33 |
| XKRX | 16.06 | 71.84 | 90.2 | 115.05 | 101.81 |
| YIPF1 | 310.29 | 351.68 | 285.04 | 354.44 | 456.27 |
| YIPF6 | 342.01 | 687.07 | 705.14 | 1078.09 | 793.2 |
| ZBED2 | 87.53 | 94.86 | 522.15 | 230.51 | 1238.63 |
| ZBTB38 | 1986.89 | 5405.41 | 3134.97 | 6174.05 | 4680.43 |
| ZC3H12C | 123.76 | 159.39 | 518.54 | 1191.95 | 985.54 |
| ZG16B | 3.42 | 17.03 | 15.46 | 32.31 | 32.59 |
| ZMAT3 | 529.91 | 925.46 | 822.66 | 1077.17 | 1234.3 |
| ZMYND8 | 585.94 | 675.31 | 711.84 | 850.29 | 1131.01 |
| ZNF280C | 181.86 | 444.81 | 326.28 | 635.21 | 467.78 |
| ZNF280D | 698.54 | 973.93 | 616.48 | 1061.55 | 1290.04 |
| ZNF282 | 374.36 | 1273.4 | 2253.55 | 2562.43 | 3165.99 |
| ZNF334 | 6.95 | 26.52 | 17.53 | 40.03 | 100.33 |
| ZWINT | 60.55 | 73.28 | 101.03 | 87.1 | 105.4 |

Altogether, the data show that Treg cells display the most pronounced differences in transcripts expression among CD4+ T cell subsets infiltrating normal and tumor tissues.

The inventors defined a subset of signature genes that describe the specific gene expression profile of tumor infiltrating Treg cells.

### Gene Signature of Tumor-Infiltrating Treg Cells is Present in Primary and Metastatic Human Tumors

The inventors then looked at the single cell level for the differential expression profile of signature genes of tumor infiltrating Treg cells. The inventors isolated CD4+ T cells from CRC and 5 NSCLC tumor samples as well as from 5 PBMCs of healthy individuals (Table II), purified Treg cells, and using an automated microfluidic system (Cl Fluidigm) captured single cells (a total of 858 Treg cells: 320 from CRC and 286 from NSCLC; 252 from PBMCs of healthy individuals). The inventors then assessed by high throughput RT-qPCR (Biomark HD, Fluidigm) the expression of 79 genes selected among the highly expressed (>10 FKPM) tumor Treg cell signature genes (FIGS. 3A, 3C and 7). Notably, it was found that the vast majority (75 over 79; 95%) of the tumor-infiltrating Treg cell signatures were co-expressed with bona fide Treg cell markers (i.e., FOXP3+ and IL2RA) (FIG. 3B). The percentage of co-expression between these Treg cell markers and the 79 genes selected among the tumor-infiltrating-Treg-cell signature genes ranged between 81% of TIGIT and 0.59% of CGA (FIG. 3B). The expression of Treg signature genes in the RNA-seq of the whole Treg cell population correlated with the percentage of single cells expressing the different genes (FIG. 3C). In order to reduce the “drop-out” effect of the single cell data (i.e., events in which a transcript is detected in one cell but not in another one because the transcript is ‘missed’ during the reverse-transcription step) (Kharchenko et al., 2014), a threshold (median value t=8.4%) was defined based on the expression distribution for each transcript and discarded genes below this threshold. The forty-five signature transcripts of tumor infiltrating Treg cells detected above this threshold were in most cases significantly over-expressed in Treg cells from both tumors (39 over 45, 87%; Wilcoxon Mann Whitney test p<0.05) or in one tumor type (43 over 45, 96%; FIG. 3D). Homogeneity of the purified tissue infiltrating Treg cells can be affected by the carry-over of cells from other lymphocyte subsets. To quantitate this possible contamination, the single cell RT-qPCR analyses of Treg cells was performed including markers specific for other lymphocytes subsets (i.e., Th1, Th2, Th17, Tfh, CD8 T cells, B cells) (FIG. 7). Our data showed that only a very low fraction of the purified single cells displayed markers of lymphocytes subsets different from Treg cells (FIG. 7).

The overlap between the signature genes in the CRC and NSCLC infiltrating Treg cells (FIG. 2) prompted us to assess whether this signature were also enriched in Treg cells infiltrating other tumors. RNA was thus extracted from Treg cells infiltrating breast cancer, gastric cancer, brain metastasis of NSCLC, and liver metastasis of CRC. It was found by RT-qPCR that tumor infiltrating Treg signatures genes were mostly upregulated also in these tumors (FIG. 3E).

Overall these data show that the tumor-infiltrating Treg cell signature genes are co-expressed at single cell level with FOXP3 and IL2RA and that several primary and metastatic human tumors express the tumor-infiltrating Treg cell signature.

### Gene Signature of Tumor Infiltrating Treg Cells is Translated in a Protein Signature

The inventors then assessed at the single cell level by flow cytometry the protein expression of ten representative signature genes present in CRC and NSCLC infiltrating Treg cells, adjacent normal tissues, and patients PBMCs. Of the ten proteins, two are proteins (OX40 and TIGIT) whose relevance for Treg cells biology has been demonstrated (Joller et al., 2014; Voo et al., 2013), seven are proteins (BATF, CCR8, CD30, IL-1 R2, IL-21R, PDL-1 and PDL-2) whose expression has never been described in tumor-infiltrating Treg cells, and one protein, 4-1BB, is a co-stimulatory receptor expressed on several hematopoietic cells, whose expression on Treg cells has been shown to mark antigen-activated cells (Schoenbrunn et al., 2012). Our findings showed that all these proteins were upregulated (FIGS. 4A and 4B), at different extent, in tumor infiltrating Treg cells compared to the Treg cells resident in normal tissues.

Altogether, our data show there is a molecular signature of tumor infiltrating Treg cells, which can be detected both at the mRNA and at the protein levels.

Expression of Tumor Treg Signature Genes is Negatively Correlated with Patients Survival

In an attempt to correlate our findings with clinical outcome, the inventors asked whether the expression of the tumor-Treg signature transcripts correlated with disease prognosis in CRC and NSCLC patients. The inventors therefore interrogated for expression of Treg signature genes transcriptomic datasets obtained from resected tumor tissues of a cohort of 177 CRC patients (GSE17536 (Smith et al., 2010) and of a cohort of 263 NSCLC patients (GSE41271—(Sato et al., 2013), and correlated high and low gene expression levels with the 5-years survival data. Among those genes whose expression is highly enriched in tumor infiltrating Treg cells, LAYN, MAGEH1 and CCR8 were selected as they are the three genes more selectively expressed (FIG. 9A-C). To normalize for differences in T cell densities within the resected tumor tissues, the inventors used the ratio between expression of the selected signature genes and CD3G. Remarkably, it was found that high expression of the three signature genes is in all cases correlated with a significantly reduced survival (FIG. 5A). Interestingly, it was also observed that expressions of the three signature genes increased with tumor staging of CRC patients (FIG. 5B).

In conclusion, high expression in the whole tumor samples of three genes (LAYN, MAGEH1 and CCR8) that are specifically and highly expressed in tumor infiltrating Treg cells, correlates with a poor prognosis in both NSCLC and CRC patients.

### Selection of Potential Targets Specifically Over-Expressed on the Surface of Tumor-Infiltrating Treg

All annotated protein isoforms encoded by the 328 genes and retrievable in the public database EnsEMBL (http://www.ensembl.org) were simultaneously analysed with the four prediction algorithms and genes encoding at least one isoform predicted to be surface exposed were considered as potential targets.

Out of 328 genes, 193 encode for at least one potential cell surface protein isoform on the basis of at least one of the four predictors. The list of protein isoforms predicted to be membrane-associated is reported in Table VI.

TABLE VI

| | | | | | SEQ ID |
|---|---|---|---|---|---|
| | | | | | No of |
| | | | | | the aa |
| | | | | | sequence |
| | | | | | of the |
| Gene | | ENSG ID | | | protein |
| name | Description | release87 | ENST ID | ENSP ID | isoform |
| LAYN | Layilin | ENSG00000204381 | ENST00000375614 | ENSP00000364764 | 1 |
| | | | ENST00000375615 | ENSP00000364765 | 2 |
| | | | ENST00000436913 | ENSP00000392942 | 3 |
| | | | ENST00000525126 | ENSP00000434328 | 4 |
| | | | ENST00000525866 | ENSP00000434300 | 5 |
| | | | ENST00000528924 | ENSP00000486561 | 6 |
| | | | ENST00000530962 | ENSP00000431627 | 7 |
| | | | ENST00000533265 | ENSP00000434972 | 8 |
| | | | ENST00000533999 | ENSP00000432434 | 9 |
| CCR8 | C—C chemokine receptor | ENSG00000179934 | ENST00000326306 | ENSP00000326432 | 10 |
| | type 8 | | ENST00000414803 | ENSP00000390104 | 11 |
| IL21R | Interleukin-21 receptor | ENSG00000103522 | ENST00000337929 | ENSP00000338010 | 12 |
| | | | ENST00000395754 | ENSP00000379103 | 13 |
| | | | ENST00000564089 | ENSP00000456707 | 14 |
| FUCA2 | Plasma alpha-L- | ENSG00000001036 | ENST00000002165 | ENSP00000002165 | 15 |
| | fucosidase | | ENST00000451668 | ENSP00000398119 | 16 |
| ICA1 | Islet cell autoantigen 1 | ENSG00000003147 | ENST00000407906 | ENSP00000386021 | 17 |
| COX10 | Protoheme IX | ENSG00000006695 | ENST00000261643 | ENSP00000261643 | 18 |
| | farnesyltransferase, mit. | | | | |
| IL32 | Interleukin-32 | ENSG00000008517 | ENST00000008180 | ENSP00000008180 | 19 |
| | | | ENST00000396890 | ENSP00000380099 | 20 |
| | | | ENST00000525228 | ENSP00000431740 | 21 |
| | | | ENST00000525377 | ENSP00000433866 | 22 |
| | | | ENST00000530890 | ENSP00000433747 | 23 |
| | | | ENST00000534507 | ENSP00000431775 | 24 |
| | | | ENST00000548246 | ENSP00000447979 | 25 |
| | | | ENST00000548476 | ENSP00000449483 | 26 |
| | | | ENST00000548807 | ENSP00000448354 | 27 |
| | | | ENST00000551513 | ENSP00000449147 | 28 |
| | | | ENST00000552356 | ENSP00000446978 | 29 |
| | | | ENST00000552936 | ENSP00000447033 | 30 |
| ETV7 | Transcription factor | ENSG00000010030 | ENST00000339796 | ENSP00000342260 | 31 |
| | ETV7 | | ENST00000627426 | ENSP00000486712 | 32 |
| ATP2C1 | Calcium-transporting | ENSG00000017260 | ENST00000328560 | ENSP00000329664 | 33 |
| | ATPase type 2C member 1 | | ENST00000359644 | ENSP00000352665 | 34 |
| | | | ENST00000422190 | ENSP00000402677 | 35 |
| | | | ENST00000428331 | ENSP00000395809 | 36 |
| | | | ENST00000504381 | ENSP00000425320 | 37 |
| | | | ENST00000504571 | ENSP00000422489 | 38 |
| | | | ENST00000504612 | ENSP00000425228 | 39 |
| | | | ENST00000504948 | ENSP00000423330 | 40 |
| | | | ENST00000505072 | ENSP00000427625 | 41 |
| | | | ENST00000505330 | ENSP00000423774 | 42 |
| | | | ENST00000507194 | ENSP00000427087 | 43 |
| | | | ENST00000507488 | ENSP00000421326 | 44 |
| | | | ENST00000508297 | ENSP00000421261 | 45 |
| | | | ENST00000508532 | ENSP00000424783 | 46 |
| | | | ENST00000508660 | ENSP00000424930 | 47 |
| | | | ENST00000509662 | ENSP00000426849 | 48 |
| | | | ENST00000510168 | ENSP00000427461 | 49 |
| | | | ENST00000513801 | ENSP00000422872 | 50 |
| | | | ENST00000515854 | ENSP00000422890 | 51 |
| | | | ENST00000533801 | ENSP00000432956 | 52 |
| FAS | Fatty acid synthase | ENSG00000026103 | ENST00000352159 | ENSP00000345601 | 53 |
| | | | ENST00000355279 | ENSP00000347426 | 54 |
| | | | ENST00000355740 | ENSP00000347979 | 55 |
| | | | ENST00000357339 | ENSP00000349896 | 56 |
| | | | ENST00000479522 | ENSP00000424113 | 57 |
| | | | ENST00000484444 | ENSP00000420975 | 58 |
| | | | ENST00000488877 | ENSP00000425159 | 59 |
| | | | ENST00000492756 | ENSP00000422453 | 60 |
| | | | ENST00000494410 | ENSP00000423755 | 61 |
| | | | ENST00000612663 | ENSP00000477997 | 62 |
| PEX3 | Peroxisomal biogenesis | ENSG00000034693 | ENST00000367591 | ENSP00000356563 | 63 |
| | factor 3 | | ENST00000367592 | ENSP00000356564 | 64 |
| TSPAN17 | Tetraspanin-17 | ENSG00000048140 | ENST00000298564 | ENSP00000298564 | 65 |
| | | | ENST00000310032 | ENSP00000309036 | 66 |
| | | | ENST00000503030 | ENSP00000425975 | 67 |
| | | | ENST00000503045 | ENSP00000425212 | 68 |
| | | | ENST00000504168 | ENSP00000423957 | 69 |
| | | | ENST00000507471 | ENSP00000423610 | 70 |
| | | | ENST00000508164 | ENSP00000422053 | 71 |
| | | | ENST00000515708 | ENSP00000426650 | 72 |
| COL9A2 | Collagen alpha-2(IX) | ENSG00000049089 | ENST00000372736 | ENSP00000361821 | 73 |
| | chain | | ENST00000372748 | ENSP00000361834 | 74 |
| | | | ENST00000417105 | ENSP00000388493 | 75 |
| NFE2L3 | Nuclear factor erythroid | ENSG00000050344 | ENST00000056233 | ENSP00000056233 | 76 |
| | 2-related factor 3 | | | | |
| TNIP3 | TNFAIP3-interacting | ENSG00000050730 | ENST00000515036 | ENSP00000424284 | 77 |
| | prot.3 | | | | |
| LY75 | Lymphocyte antigen 75 | ENSG00000054219 | ENST00000263636 | ENSP00000263636 | 78 |
| YIPF1 | Protein YIPF1 | ENSG00000058799 | ENST00000072644 | ENSP00000072644 | 79 |
| | | | ENST00000371399 | ENSP00000360452 | 80 |
| | | | ENST00000412288 | ENSP00000416507 | 81 |
| | | | ENST00000464950 | ENSP00000432266 | 82 |
| ISOC1 | Isochorismatase domain- | ENSG00000066583 | ENST00000173527 | ENSP00000173527 | 83 |
| | containing protein 1 | | ENST00000514194 | ENSP00000421273 | 84 |
| ACSL4 | Long-chain-fatty-acid-- | ENSG00000068366 | ENST00000340800 | ENSP00000339787 | 85 |
| | CoA ligase 4 | | ENST00000469796 | ENSP00000419171 | 86 |
| | | | ENST00000469857 | ENSP00000423077 | 87 |
| | | | ENST00000502391 | ENSP00000425408 | 88 |
| | | | ENST00000504980 | ENSP00000421425 | 89 |
| | | | ENST00000508092 | ENSP00000425378 | 90 |
| MAST4 | Microtubule-assoc.serine/ | ENSG00000069020 | ENST00000434115 | ENSP00000396765 | 91 |
| | threonine-protein kinase 4 | | | | |
| LMCD1 | LIM and cysteine-rich | ENSG00000071282 | ENST00000456506 | ENSP00000405049 | 92 |
| | domains protein 1 | | | | |
| TFRC | Transferrin receptor | ENSG00000072274 | ENST00000360110 | ENSP00000353224 | 93 |
| | protein 1 | | ENST00000392396 | ENSP00000376197 | 94 |
| | | | ENST00000421258 | ENSP00000402839 | 95 |
| | | | ENST00000426789 | ENSP00000414015 | 96 |
| PANX2 | Pannexin-2 | ENSG00000073150 | ENST00000159647 | ENSP00000159647 | 97 |
| | | | ENST00000395842 | ENSP00000379183 | 98 |
| | | | ENST00000402472 | ENSP00000384148 | 99 |
| FNDC3B | Fibronectin type III | ENSG00000075420 | ENST00000336824 | ENSP00000338523 | 100 |
| | domain-containing | | ENST00000415807 | ENSP00000411242 | 101 |
| | protein 3B | | ENST00000416957 | ENSP00000389094 | 102 |
| | | | ENST00000421757 | ENSP00000408496 | 103 |
| | | | ENST00000423424 | ENSP00000392471 | 104 |
| IL12RB2 | Interleukin-12 receptor | ENSG00000081985 | ENST00000262345 | ENSP00000262345 | 105 |
| | subunit beta-2 | | ENST00000371000 | ENSP00000360039 | 106 |
| | | | ENST00000441640 | ENSP00000400959 | 107 |
| | | | ENST00000541374 | ENSP00000445276 | 108 |
| | | | ENST00000544434 | ENSP00000442443 | 109 |
| STARD7 | StAR-related lipid | ENSG00000084090 | ENST00000337288 | ENSP00000338030 | 110 |
| | transfer protein 7, | | | | |
| | mitochondrial | | | | |
| SSH1 | Protein phosphatase | ENSG00000084112 | ENST00000546697 | ENSP00000446652 | 111 |
| | Slingshot homolog 1 | | ENST00000548522 | ENSP00000448586 | 112 |
| MGST2 | Microsomal glutathione | ENSG00000085871 | ENST00000265498 | ENSP00000265498 | 113 |
| | S-transferase 2 | | ENST00000503816 | ENSP00000423008 | 114 |
| | | | ENST00000506797 | ENSP00000424278 | 115 |
| | | | ENST00000616265 | ENSP00000482639 | 116 |
| ACOX3 | Peroxisomal acyl- | ENSG00000087008 | ENST00000514423 | ENSP00000427321 | 117 |
| | coenzyme A oxidase 3 | | | | |
| ANKRD10 | Ankyrin repeat domain- | ENSG00000088448 | ENST00000603993 | ENSP00000474638 | 118 |
| | containing protein 10 | | | | |
| FKBP1A | Peptidyl-prolyl cis-trans | ENSG00000088832 | ENST00000612074 | ENSP00000480846 | 119 |
| | isomerase FKBP1A | | ENST00000614856 | ENSP00000482758 | 120 |
| | | | ENST00000618612 | ENSP00000478093 | 121 |
| SIRPG | Signal-regulatory protein | ENSG00000089012 | ENST00000216927 | ENSP00000216927 | 122 |
| | gamma | | ENST00000303415 | ENSP00000305529 | 123 |
| | | | ENST00000344103 | ENSP00000342759 | 124 |
| | | | ENST00000381580 | ENSP00000370992 | 125 |
| | | | ENST00000381583 | ENSP00000370995 | 126 |
| WHRN | Whirlin | ENSG00000095397 | ENST00000374059 | ENSP00000363172 | 127 |
| CENPM | Centromere protein M | ENSG00000100162 | ENST00000215980 | ENSP00000215980 | 128 |
| | | | ENST00000402338 | ENSP00000384731 | 129 |
| | | | ENST00000402420 | ENSP00000384132 | 130 |
| | | | ENST00000404067 | ENSP00000384814 | 131 |
| | | | ENST00000407253 | ENSP00000384743 | 132 |
| NCF4 | Neutrophil cytosol factor 4 | ENSG00000100365 | ENST00000447071 | ENSP00000414958 | 133 |
| CSF2RB | Cytokine receptor | ENSG00000100368 | ENST00000262825 | ENSP00000262825 | 134 |
| | common subunit beta | | ENST00000403662 | ENSP00000384053 | 135 |
| | | | ENST00000406230 | ENSP00000385271 | 136 |
| | | | ENST00000421539 | ENSP00000393585 | 137 |
| CNIH1 | Protein cornichon | ENSG00000100528 | ENST00000216416 | ENSP00000216416 | 138 |
| | homolog 1 | | ENST00000395573 | ENSP00000378940 | 139 |
| | | | ENST00000553660 | ENSP00000452457 | 140 |
| | | | ENST00000554683 | ENSP00000452466 | 141 |
| | | | ENST00000556113 | ENSP00000451142 | 142 |
| | | | ENST00000557659 | ENSP00000451640 | 143 |
| | | | ENST00000557690 | ENSP00000451852 | 144 |
| PIGU | Phosphatidylinositol | ENSG00000101464 | ENST00000217446 | ENSP00000217446 | 145 |
| | glycan anchor | | ENST00000374820 | ENSP00000363953 | 146 |
| | biosynthesis class U | | ENST00000438215 | ENSP00000395755 | 147 |
| | protein | | | | |
| NDFIP2 | NEDD4 family- | ENSG00000102471 | ENST00000218652 | ENSP00000218652 | 148 |
| | interacting protein 2 | | ENST00000487865 | ENSP00000419200 | 149 |
| | | | ENST00000612570 | ENSP00000480798 | 150 |
| | | | ENST00000620924 | ENSP00000480881 | 151 |
| ACP5 | Tartrate-resistant acid | ENSG00000102575 | ENST00000218758 | ENSP00000218758 | 152 |
| | phosphatase type 5 | | ENST00000412435 | ENSP00000392374 | 153 |
| | | | ENST00000433365 | ENSP00000413456 | 154 |
| | | | ENST00000589792 | ENSP00000468685 | 155 |
| | | | ENST00000590420 | ENSP00000468509 | 156 |
| | | | ENST00000590832 | ENSP00000465127 | 157 |
| | | | ENST00000591319 | ENSP00000464831 | 158 |
| | | | ENST00000592828 | ENSP00000468767 | 159 |
| NFAT5 | Nuclear factor of | ENSG00000102908 | ENST00000567990 | ENSP00000455115 | 160 |
| | activated T-cells 5 | | | | |
| CYB5B | Cytochrome b5 type B | ENSG00000103018 | ENST00000307892 | ENSP00000308430 | 161 |
| | | | ENST00000512062 | ENSP00000423679 | 162 |
| | | | ENST00000568237 | ENSP00000464102 | 163 |
| LAPTM4B | Lysosomal-associated | ENSG00000104341 | ENST00000445593 | ENSP00000402301 | 164 |
| | transmembrane protein 4B | | ENST00000517924 | ENSP00000429868 | 165 |
| | | | ENST00000521545 | ENSP00000428409 | 166 |
| | | | ENST00000619747 | ENSP00000482533 | 167 |
| IL7 | Interleukin-7 | ENSG00000104432 | ENST00000263851 | ENSP00000263851 | 168 |
| | | | ENST00000379113 | ENSP00000368408 | 169 |
| | | | ENST00000518982 | ENSP00000430272 | 170 |
| | | | ENST00000520215 | ENSP00000428364 | 171 |
| | | | ENST00000520269 | ENSP00000427750 | 172 |
| | | | ENST00000520317 | ENSP00000427800 | 173 |
| | | | ENST00000541183 | ENSP00000438922 | 174 |
| EBI3 | Interleukin-27 subunit | ENSG00000105246 | ENST00000221847 | ENSP00000221847 | 175 |
| | beta | | | | |
| PLA2G4C | Cytosolic phospholipase | ENSG00000105499 | ENST00000595161 | ENSP00000469528 | 176 |
| | A2 gamma | | ENST00000595487 | ENSP00000471328 | 177 |
| | | | ENST00000596352 | ENSP00000471759 | 178 |
| | | | ENST00000598488 | ENSP00000468972 | 179 |
| GLCCI1 | Glucocorticoid-induced | ENSG00000106415 | ENST00000430798 | ENSP00000396171 | 180 |
| | transcript 1 protein | | | | |
| MINPP1 | Multiple inositol | ENSG00000107789 | ENST00000371994 | ENSP00000361062 | 181 |
| | polyphosphate | | ENST00000371996 | ENSP00000361064 | 182 |
| | phosphatase 1 | | ENST00000536010 | ENSP00000437823 | 183 |
| WSB1 | WD repeat and SOCS | ENSG00000109046 | ENST00000581440 | ENSP00000462737 | 184 |
| | box-containing protein 1 | | ENST00000582208 | ENSP00000463621 | 185 |
| | | | ENST00000583193 | ENSP00000462595 | 186 |
| | | | ENST00000583742 | ENSP00000462365 | 187 |
| HTATIP2 | Oxidoreductase HTATIP2 | ENSG00000109854 | ENST00000419348 | ENSP00000392985 | 188 |
| | | | ENST00000530266 | ENSP00000436548 | 189 |
| | | | ENST00000532081 | ENSP00000432107 | 190 |
| | | | ENST00000532505 | ENSP00000432338 | 191 |
| CTSC | Dipeptidyl peptidase 1 | ENSG00000109861 | ENST00000227266 | ENSP00000227266 | 192 |
| | | | ENST00000524463 | ENSP00000432541 | 193 |
| | | | ENST00000527018 | ENSP00000432556 | 194 |
| | | | ENST00000528020 | ENSP00000433229 | 195 |
| | | | ENST00000529974 | ENSP00000433539 | 196 |
| VWA5A | von Willebrand factor A | ENSG00000110002 | ENST00000392744 | ENSP00000376501 | 197 |
| | domain-containing | | ENST00000392748 | ENSP00000376504 | 198 |
| | protein 5A | | ENST00000456829 | ENSP00000407726 | 199 |
| SLC35F2 | Solute carrier family 35 | ENSG00000110660 | ENST00000375682 | ENSP00000364834 | 200 |
| | member F2 | | ENST00000525071 | ENSP00000434307 | 201 |
| | | | ENST00000525815 | ENSP00000436785 | 202 |
| | | | ENST00000532513 | ENSP00000433783 | 203 |
| VDR | Vitamin D3 receptor | ENSG00000111424 | ENST00000547065 | ENSP00000449074 | 204 |
| SEC24A | Protein transport protein | ENSG00000113615 | ENST00000398844 | ENSP00000381823 | 205 |
| | Sec24A | | | | |
| IL1R2 | Interleukin-1 receptor | ENSG00000115590 | ENST00000332549 | ENSP00000330959 | 206 |
| | type 2 | | ENST00000393414 | ENSP00000377066 | 207 |
| | | | ENST00000441002 | ENSP00000414611 | 208 |
| | | | ENST00000457817 | ENSP00000408415 | 209 |
| IL1R1 | Interleukin-1 receptor | ENSG00000115594 | ENST00000409288 | ENSP00000386478 | 210 |
| | type 1 | | ENST00000409329 | ENSP00000387131 | 211 |
| | | | ENST00000409589 | ENSP00000386555 | 212 |
| | | | ENST00000409929 | ENSP00000386776 | 213 |
| | | | ENST00000410023 | ENSP00000386380 | 214 |
| | | | ENST00000413623 | ENSP00000407017 | 215 |
| | | | ENST00000422532 | ENSP00000390349 | 216 |
| | | | ENST00000424272 | ENSP00000415366 | 217 |
| | | | ENST00000428279 | ENSP00000410461 | 218 |
| | | | ENST00000430171 | ENSP00000408101 | 219 |
| | | | ENST00000442590 | ENSP00000393296 | 220 |
| | | | ENST00000450319 | ENSP00000411627 | 221 |
| | | | ENST00000452403 | ENSP00000401646 | 222 |
| IL1RL2 | Interleukin-1 receptor- | ENSG00000115598 | ENST00000264257 | ENSP00000264257 | 223 |
| | like 2 | | ENST00000421464 | ENSP00000387611 | 224 |
| | | | ENST00000441515 | ENSP00000413348 | 225 |
| IL1RL1 | Interleukin-1 receptor- | ENSG00000115602 | ENST00000233954 | ENSP00000233954 | 226 |
| | like 1 | | ENST00000311734 | ENSP00000310371 | 227 |
| | | | ENST00000404917 | ENSP00000384822 | 228 |
| | | | ENST00000409584 | ENSP00000386618 | 229 |
| | | | ENST00000427077 | ENSP00000391120 | 230 |
| | | | ENST00000447231 | ENSP00000409437 | 231 |
| UXS1 | UDP-glucuronic acid | ENSG00000115652 | ENST00000283148 | ENSP00000283148 | 232 |
| | decarboxylase 1 | | ENST00000409501 | ENSP00000387019 | 233 |
| | | | ENST00000441952 | ENSP00000416656 | 234 |
| | | | ENST00000457835 | ENSP00000399316 | 235 |
| SLC25A12 | Calcium-binding | ENSG00000115840 | ENST00000426896 | ENSP00000413968 | 236 |
| | mitochondrial carrier | | | | |
| | protein Aralar1 | | | | |
| THADA | Thyroid adenoma- | ENSG00000115970 | ENST00000403856 | ENSP00000385469 | 237 |
| | associated protein | | | | |
| LEPR | Leptin receptor | ENSG00000116678 | ENST00000344610 | ENSP00000340884 | 238 |
| | | | ENST00000349533 | ENSP00000330393 | 239 |
| | | | ENST00000371058 | ENSP00000360097 | 240 |
| | | | ENST00000371059 | ENSP00000360098 | 241 |
| | | | ENST00000371060 | ENSP00000360099 | 242 |
| | | | ENST00000406510 | ENSP00000384025 | 243 |
| | | | ENST00000616738 | ENSP00000483390 | 244 |
| MREG | Melanoregulin | ENSG00000118242 | ENST00000263268 | ENSP00000263268 | 245 |
| | | | ENST00000620139 | ENSP00000484331 | 246 |
| FLVCR2 | Feline leukemia virus | ENSG00000119686 | ENST00000238667 | ENSP00000238667 | 247 |
| | subgroup C receptor- | | ENST00000539311 | ENSP00000443439 | 248 |
| | related protein 2 | | ENST00000553341 | ENSP00000452584 | 249 |
| | | | ENST00000553587 | ENSP00000451603 | 250 |
| | | | ENST00000554580 | ENSP00000451781 | 251 |
| | | | ENST00000555027 | ENSP00000452453 | 252 |
| | | | ENST00000555058 | ENSP00000451104 | 253 |
| | | | ENST00000556856 | ENSP00000452468 | 254 |
| SOCS2 | Suppressor of cytokine | ENSG00000120833 | ENST00000548537 | ENSP00000448709 | 255 |
| | signaling 2 | | ENST00000549510 | ENSP00000474888 | 256 |
| RDH10 | Retinol dehydrogenase 10 | ENSG00000121039 | ENST00000240285 | ENSP00000240285 | 257 |
| | | | ENST00000519380 | ENSP00000428132 | 258 |
| | | | ENST00000521928 | ENSP00000429727 | 259 |
| LAX1 | Lymphocyte | ENSG00000122188 | ENST00000367217 | ENSP00000356186 | 260 |
| | transmembrane adapter 1 | | ENST00000442561 | ENSP00000406970 | 261 |
| ZWINT | ZW10 interactor | ENSG00000122952 | ENST00000489649 | ENSP00000473330 | 262 |
| ACOT9 | Acyl-coenzyme A | ENSG00000123130 | ENST00000336430 | ENSP00000336580 | 263 |
| | thioesterase 9, | | ENST00000379303 | ENSP00000368605 | 264 |
| | mitochondrial | | ENST00000494361 | ENSP00000420238 | 265 |
| TM9SF2 | Transmembrane 9 | ENSG00000125304 | ENST00000376387 | ENSP00000365567 | 266 |
| | superfamily member 2 | | | | |
| HS3ST3B1 | Heparan sulfate | ENSG00000125430 | ENST00000360954 | ENSP00000354213 | 267 |
| | glucosamine 3-O- | | ENST00000466596 | ENSP00000436078 | 268 |
| | sulfotransferase 3B1 | | | | |
| EML2 | Echinoderm | ENSG00000125746 | ENST00000245925 | ENSP00000245925 | 269 |
| | microtubule-associated | | ENST00000586195 | ENSP00000465339 | 270 |
| | protein-like 2 | | ENST00000586405 | ENSP00000465885 | 271 |
| | | | ENST00000586770 | ENSP00000465786 | 272 |
| | | | ENST00000587152 | ENSP00000468312 | 273 |
| | | | ENST00000587484 | ENSP00000465994 | 274 |
| | | | ENST00000588272 | ENSP00000466100 | 275 |
| | | | ENST00000588308 | ENSP00000468329 | 276 |
| | | | ENST00000589876 | ENSP00000464789 | 277 |
| | | | ENST00000590018 | ENSP00000468373 | 278 |
| | | | ENST00000590043 | ENSP00000464804 | 279 |
| | | | ENST00000590819 | ENSP00000464950 | 280 |
| | | | ENST00000591721 | ENSP00000468470 | 281 |
| | | | ENST00000592853 | ENSP00000468383 | 282 |
| | | | ENST00000593255 | ENSP00000467941 | 283 |
| MGME1 | Mitochondrial genome | ENSG00000125871 | ENST00000377704 | ENSP00000366933 | 284 |
| | maintenance | | ENST00000377709 | ENSP00000366938 | 285 |
| | exonuclease 1 | | ENST00000377710 | ENSP00000366939 | 286 |
| IGFLR1 | IGF-like family receptor 1 | ENSG00000126246 | ENST00000246532 | ENSP00000246532 | 287 |
| | | | ENST00000588018 | ENSP00000468545 | 288 |
| | | | ENST00000588992 | ENSP00000465962 | 289 |
| | | | ENST00000591277 | ENSP00000468644 | 290 |
| | | | ENST00000591748 | ENSP00000476009 | 291 |
| | | | ENST00000592537 | ENSP00000466181 | 292 |
| | | | ENST00000592693 | ENSP00000474913 | 293 |
| | | | ENST00000592889 | ENSP00000467750 | 294 |
| MYO5C | Unconventional myosin- | ENSG00000128833 | ENST00000261839 | ENSP00000261839 | 295 |
| | Vc | | | | |
| ITFG1 | T-cell | ENSG00000129636 | ENST00000320640 | ENSP00000319918 | 296 |
| | immunomodulatory | | ENST00000544001 | ENSP00000441062 | 297 |
| | protein | | ENST00000563730 | ENSP00000455630 | 298 |
| | | | ENST00000565262 | ENSP00000457665 | 299 |
| | | | ENST00000565940 | ENSP00000459192 | 300 |
| SYT11 | Synaptotagmin-11 | ENSG00000132718 | ENST00000368324 | ENSP00000357307 | 301 |
| SLC41A1 | Solute carrier family 41 | ENSG00000133065 | ENST00000367137 | ENSP00000356105 | 302 |
| | member 1 | | | | |
| ATP13A3 | Probable cation- | ENSG00000133657 | ENST00000256031 | ENSP00000256031 | 303 |
| | transporting ATPase | | ENST00000429136 | ENSP00000402550 | 304 |
| | 13A3 | | ENST00000439040 | ENSP00000416508 | 305 |
| | | | ENST00000446356 | ENSP00000410767 | 306 |
| | | | ENST00000457986 | ENSP00000406234 | 307 |
| | | | ENST00000619199 | ENSP00000482200 | 308 |
| MICAL2 | Protein-methionine | ENSG00000133816 | ENST00000379612 | ENSP00000368932 | 309 |
| | sulfoxide oxidase | | | | |
| | MICAL2 | | | | |
| CABLES1 | CDK5 and ABL1 enzyme | ENSG00000134508 | ENST00000256925 | ENSP00000256925 | 310 |
| | substrate 1 | | ENST00000579963 | ENSP00000464435 | 311 |
| HAVCR2 | Hepatitis A virus cellular | ENSG00000135077 | ENST00000307851 | ENSP00000312002 | 312 |
| | receptor 2 | | ENST00000522593 | ENSP00000430873 | 313 |
| CGA | Chromogranin-A | ENSG00000135346 | ENST00000369582 | ENSP00000358595 | 314 |
| | | | ENST00000610310 | ENSP00000482232 | 315 |
| | | | ENST00000625577 | ENSP00000486666 | 316 |
| | | | ENST00000627148 | ENSP00000486024 | 317 |
| | | | ENST00000630630 | ENSP00000487300 | 318 |
| FAIM2 | Protein lifeguard 2 | ENSG00000135472 | ENST00000320634 | ENSP00000321951 | 319 |
| | | | ENST00000547871 | ENSP00000449360 | 320 |
| | | | ENST00000550195 | ENSP00000447715 | 321 |
| | | | ENST00000550635 | ENSP00000449711 | 322 |
| | | | ENST00000550890 | ENSP00000450132 | 323 |
| | | | ENST00000552669 | ENSP00000446771 | 324 |
| | | | ENST00000552863 | ENSP00000449957 | 325 |
| ARHGEF4 | Rho guanine nucleotide | ENSG00000136002 | ENST00000392953 | ENSP00000376680 | 326 |
| | exchange factor 4 | | | | |
| SLC41A2 | Solute carrier family 41 | ENSG00000136052 | ENST00000258538 | ENSP00000258538 | 327 |
| | member 2 | | ENST00000437220 | ENSP00000391377 | 328 |
| NUSAP1 | Nucleolar and spindle- | ENSG00000137804 | ENST00000557840 | ENSP00000453428 | 329 |
| | associated protein 1 | | ENST00000559046 | ENSP00000452725 | 330 |
| ADAM10 | Disintegrin and | ENSG00000137845 | ENST00000260408 | ENSP00000260408 | 331 |
| | metalloproteinase | | ENST00000396136 | ENSP00000456542 | 332 |
| | domain-containing | | ENST00000402627 | ENSP00000386056 | 333 |
| | protein 10 | | ENST00000439637 | ENSP00000391930 | 334 |
| | | | ENST00000461408 | ENSP00000481779 | 335 |
| | | | ENST00000558004 | ENSP00000452704 | 336 |
| | | | ENST00000559053 | ENSP00000453952 | 337 |
| | | | ENST00000561288 | ENSP00000452639 | 338 |
| HADHB | Trifunctional enzyme | ENSG00000138029 | ENST00000545822 | ENSP00000442665 | 339 |
| | subunit beta, | | | | |
| | mitochondrial | | | | |
| CD27 | CD27 antigen | ENSG00000139193 | ENST00000266557 | ENSP00000266557 | 340 |
| CDH24 | Cadherin-24 | ENSG00000139880 | ENST00000267383 | ENSP00000267383 | 341 |
| | | | ENST00000397359 | ENSP00000380517 | 342 |
| | | | ENST00000487137 | ENSP00000434821 | 343 |
| | | | ENST00000554034 | ENSP00000452493 | 344 |
| | | | ENST00000610348 | ENSP00000478078 | 345 |
| ETFA | Electron transfer | ENSG00000140374 | ENST00000560044 | ENSP00000452942 | 346 |
| | alpha, mitochondrial | | ENST00000560309 | ENSP00000453753 | 347 |
| | flavoprotein subunit | | | | |
| KSR1 | Kinase suppressor of Ras 1 | ENSG00000141068 | ENST00000580163 | ENSP00000463204 | 348 |
| SECTM1 | Secreted and | ENSG00000141574 | ENST00000269389 | ENSP00000269389 | 349 |
| | transmembrane protein 1 | | ENST00000580437 | ENSP00000463904 | 350 |
| | | | ENST00000581691 | ENSP00000463114 | 351 |
| | | | ENST00000581864 | ENSP00000464111 | 352 |
| | | | ENST00000581954 | ENSP00000464385 | 353 |
| | | | ENST00000582290 | ENSP00000462294 | 354 |
| | | | ENST00000582563 | ENSP00000463120 | 355 |
| | | | ENST00000583093 | ENSP00000462563 | 356 |
| EVA1B | Protein eva-1 homolog B | ENSG00000142694 | ENST00000270824 | ENSP00000270824 | 357 |
| CTTNBP2NL | CTTNBP2 N-terminal-like | ENSG00000143079 | ENST00000271277 | ENSP00000271277 | 358 |
| | protein | | ENST00000441739 | ENSP00000390976 | 359 |
| CASQ1 | Calsequestrin-1 | ENSG00000143318 | ENST00000368078 | ENSP00000357057 | 360 |
| ARL6IP5 | PRA1 family protein 3 | ENSG00000144746 | ENST00000273258 | ENSP00000273258 | 361 |
| | | | ENST00000478935 | ENSP00000420138 | 362 |
| | | | ENST00000484921 | ENSP00000419374 | 363 |
| | | | ENST00000485444 | ENSP00000419021 | 364 |
| ADPRH | [Protein ADP- | ENSG00000144843 | ENST00000357003 | ENSP00000349496 | 365 |
| | ribosylarginine] | | ENST00000465513 | ENSP00000417430 | 366 |
| | hydrolase | | ENST00000478399 | ENSP00000420200 | 367 |
| | | | ENST00000478927 | ENSP00000417528 | 368 |
| | | | ENST00000481816 | ENSP00000419703 | 369 |
| PAM | Peptidyl-glycine alpha- | ENSG00000145730 | ENST00000304400 | ENSP00000306100 | 370 |
| | amidating | | ENST00000345721 | ENSP00000302544 | 371 |
| | monooxygenase | | ENST00000346918 | ENSP00000282992 | 372 |
| | | | ENST00000348126 | ENSP00000314638 | 373 |
| | | | ENST00000438793 | ENSP00000396493 | 374 |
| | | | ENST00000455264 | ENSP00000403461 | 375 |
| | | | ENST00000504691 | ENSP00000424203 | 376 |
| | | | ENST00000505654 | ENSP00000421569 | 377 |
| | | | ENST00000506006 | ENSP00000423611 | 378 |
| | | | ENST00000509832 | ENSP00000423763 | 379 |
| | | | ENST00000511477 | ENSP00000421823 | 380 |
| | | | ENST00000511839 | ENSP00000426448 | 381 |
| | | | ENST00000512073 | ENSP00000420851 | 382 |
| RNF145 | RING finger protein 145 | ENSG00000145860 | ENST00000274542 | ENSP00000274542 | 383 |
| | | | ENST00000424310 | ENSP00000409064 | 384 |
| | | | ENST00000518802 | ENSP00000430955 | 385 |
| | | | ENST00000519865 | ENSP00000430397 | 386 |
| | | | ENST00000520638 | ENSP00000429071 | 387 |
| | | | ENST00000521606 | ENSP00000430753 | 388 |
| | | | ENST00000611185 | ENSP00000482720 | 389 |
| TMEM140 | Transmembrane protein | ENSG00000146859 | ENST00000275767 | ENSP00000275767 | 390 |
| | 140 | | | | |
| CHST7 | Carbohydrate | ENSG00000147119 | ENST00000276055 | ENSP00000276055 | 391 |
| | sulfotransferase 7 | | | | |
| CHRNA6 | Neuronal acetylcholine | ENSG00000147434 | ENST00000276410 | ENSP00000276410 | 392 |
| | receptor subunit alpha-6 | | ENST00000533810 | ENSP00000434659 | 393 |
| | | | ENST00000534622 | ENSP00000433871 | 394 |
| PTPRJ | Receptor-type tyrosine- | ENSG00000149177 | ENST00000418331 | ENSP00000400010 | 395 |
| | protein phosphatase eta | | ENST00000440289 | ENSP00000409733 | 396 |
| | | | ENST00000527952 | ENSP00000435618 | 397 |
| | | | ENST00000534219 | ENSP00000432686 | 398 |
| | | | ENST00000613246 | ENSP00000477933 | 399 |
| | | | ENST00000615445 | ENSP00000479342 | 400 |
| NCAM1 | Neural cell adhesion | ENSG00000149294 | ENST00000316851 | ENSP00000318472 | 401 |
| | molecule 1 | | ENST00000401611 | ENSP00000384055 | 402 |
| | | | ENST00000524916 | ENSP00000478072 | 403 |
| | | | ENST00000526322 | ENSP00000479687 | 404 |
| | | | ENST00000528158 | ENSP00000486241 | 405 |
| | | | ENST00000528590 | ENSP00000480269 | 406 |
| | | | ENST00000529356 | ENSP00000482205 | 407 |
| | | | ENST00000531044 | ENSP00000484943 | 408 |
| | | | ENST00000531817 | ENSP00000475074 | 409 |
| | | | ENST00000533073 | ENSP00000486406 | 410 |
| | | | ENST00000613217 | ENSP00000479353 | 411 |
| | | | ENST00000615112 | ENSP00000480797 | 412 |
| | | | ENST00000615285 | ENSP00000479241 | 413 |
| | | | ENST00000618266 | ENSP00000477835 | 414 |
| | | | ENST00000619839 | ENSP00000480132 | 415 |
| | | | ENST00000620046 | ENSP00000482852 | 416 |
| | | | ENST00000621128 | ENSP00000481083 | 417 |
| | | | ENST00000621518 | ENSP00000477808 | 418 |
| | | | ENST00000621850 | ENSP00000480774 | 419 |
| INPP1 | Inositol polyphosphate | ENSG00000151689 | ENST00000413239 | ENSP00000391415 | 420 |
| | 1-phosphatase | | ENST00000444194 | ENSP00000404732 | 421 |
| | | | ENST00000451089 | ENSP00000410662 | 422 |
| | | | ENST00000458193 | ENSP00000412119 | 423 |
| JAKMIP1 | Janus kinase and | ENSG00000152969 | ENST00000409021 | ENSP00000386711 | 424 |
| | protein 1 | | ENST00000409371 | ENSP00000387042 | 425 |
| | microtubule-interacting | | | | |
| RHOC | Rho-related GTP-binding | ENSG00000155366 | ENST00000468093 | ENSP00000431392 | 426 |
| | protein RhoC | | ENST00000484280 | ENSP00000434310 | 427 |
| | | | ENST00000528831 | ENSP00000432209 | 428 |
| SLC16A1 | Monocarboxylate | ENSG00000155380 | ENST00000369626 | ENSP00000358640 | 429 |
| | transporter 1 | | ENST00000429288 | ENSP00000397106 | 430 |
| | | | ENST00000443580 | ENSP00000399104 | 431 |
| | | | ENST00000458229 | ENSP00000416167 | 432 |
| | | | ENST00000538576 | ENSP00000441065 | 433 |
| CXCL13 | C—X—C motif chemokine | ENSG00000156234 | ENST00000286758 | ENSP00000286758 | 434 |
| | 13 | | | | |
| SH3RF2 | Putative E3 ubiquitin- | ENSG00000156463 | ENST00000359120 | ENSP00000352028 | 435 |
| | protein ligase SH3RF2 | | ENST00000511217 | ENSP00000424497 | 436 |
| NPTN | Neuroplastin | ENSG00000156642 | ENST00000345330 | ENSP00000290401 | 437 |
| | | | ENST00000351217 | ENSP00000342958 | 438 |
| | | | ENST00000562924 | ENSP00000456349 | 439 |
| | | | ENST00000563691 | ENSP00000457028 | 440 |
| | | | ENST00000565325 | ENSP00000457470 | 441 |
| AHCYL2 | Adenosylhomocysteinase 3 | ENSG00000158467 | ENST00000466924 | ENSP00000419346 | 442 |
| PTGIR | Prostacyclin receptor | ENSG00000160013 | ENST00000291294 | ENSP00000291294 | 443 |
| | | | ENST00000594275 | ENSP00000469408 | 444 |
| | | | ENST00000596260 | ENSP00000468970 | 445 |
| | | | ENST00000597185 | ENSP00000470566 | 446 |
| | | | ENST00000598865 | ENSP00000470799 | 447 |
| TMPRSS3 | Transmembrane | ENSG00000160183 | ENST00000291532 | ENSP00000291532 | 448 |
| | protease serine 4 | | ENST00000398397 | ENSP00000381434 | 449 |
| | | | ENST00000398405 | ENSP00000381442 | 450 |
| | | | ENST00000433957 | ENSP00000411013 | 451 |
| FCRL3 | Fc receptor-like protein 3 | ENSG00000160856 | ENST00000368184 | ENSP00000357167 | 452 |
| | | | ENST00000368186 | ENSP00000357169 | 453 |
| | | | ENST00000477837 | ENSP00000433430 | 454 |
| | | | ENST00000485028 | ENSP00000434331 | 455 |
| | | | ENST00000492769 | ENSP00000435487 | 456 |
| | | | ENST00000496769 | ENSP00000473680 | 457 |
| PAQR4 | Progestin and adipoQ | ENSG00000162073 | ENST00000293978 | ENSP00000293978 | 458 |
| | receptor family member 4 | | ENST00000318782 | ENSP00000321804 | 459 |
| | | | ENST00000572687 | ENSP00000459418 | 460 |
| | | | ENST00000574988 | ENSP00000458683 | 461 |
| | | | ENST00000576565 | ENSP00000460326 | 462 |
| ZG16B | Zymogen granule | ENSG00000162078 | ENST00000382280 | ENSP00000371715 | 463 |
| | protein 16 homolog B | | ENST00000570670 | ENSP00000460793 | 464 |
| | | | ENST00000571723 | ENSP00000458847 | 465 |
| | | | ENST00000572863 | ENSP00000461740 | 466 |
| SGPP2 | Sphingosine-1- | ENSG00000163082 | ENST00000321276 | ENSP00000315137 | 467 |
| | phosphate phosphatase 2 | | | | |
| NEURL3 | E3 ubiquitin-protein | ENSG00000163121 | ENST00000310865 | ENSP00000479456 | 468 |
| | ligase NEURL1B | | ENST00000435380 | ENSP00000480933 | 469 |
| KIF15 | Kinesin-like protein | ENSG00000163808 | ENST00000438321 | ENSP00000406939 | 470 |
| | KIF15 | | | | |
| TMEM184C | Transmembrane protein | ENSG00000164168 | ENST00000296582 | ENSP00000296582 | 471 |
| | 184C | | ENST00000505999 | ENSP00000421159 | 472 |
| | | | ENST00000508208 | ENSP00000425940 | 473 |
| C5ORF63 | Glutaredoxin-like protein | ENSG00000164241 | ENST00000296662 | ENSP00000453964 | 474 |
| | C5orf63 | | ENST00000508527 | ENSP00000475157 | 475 |
| | | | ENST00000509733 | ENSP00000475415 | 476 |
| | | | ENST00000535381 | ENSP00000454153 | 477 |
| | | | ENST00000606042 | ENSP00000475733 | 478 |
| | | | ENST00000606937 | ENSP00000475810 | 479 |
| | | | ENST00000607731 | ENSP00000476160 | 480 |
| MELK | Maternal embryonic | ENSG00000165304 | ENST00000495529 | ENSP00000487536 | 481 |
| | leucine zipper kinase | | ENST00000536329 | ENSP00000443550 | 482 |
| | | | ENST00000536987 | ENSP00000439184 | 483 |
| | | | ENST00000543751 | ENSP00000441596 | 484 |
| | | | ENST00000626154 | ENSP00000486558 | 485 |
| FAAH2 | Fatty-acid amide | ENSG00000165591 | ENST00000374900 | ENSP00000364035 | 486 |
| | hydrolase 2 | | | | |
| TPP1 | Alpha-tocopherol | ENSG00000166340 | ENST00000299427 | ENSP00000299427 | 487 |
| | transfer protein | | ENST00000436873 | ENSP00000398136 | 488 |
| | | | ENST00000528571 | ENSP00000434647 | 489 |
| | | | ENST00000528657 | ENSP00000435001 | 490 |
| CX3CR1 | CX3C chemokine | ENSG00000168329 | ENST00000358309 | ENSP00000351059 | 491 |
| | receptor 1 | | ENST00000399220 | ENSP00000382166 | 492 |
| | | | ENST00000412814 | ENSP00000408835 | 493 |
| | | | ENST00000435290 | ENSP00000394960 | 494 |
| | | | ENST00000541347 | ENSP00000439140 | 495 |
| | | | ENST00000542107 | ENSP00000444928 | 496 |
| TSPAN5 | Tetraspanin-5 | ENSG00000168785 | ENST00000305798 | ENSP00000307701 | 497 |
| | | | ENST00000505184 | ENSP00000423916 | 498 |
| | | | ENST00000508798 | ENSP00000421808 | 499 |
| | | | ENST00000511651 | ENSP00000426248 | 500 |
| | | | ENST00000511800 | ENSP00000422548 | 501 |
| | | | ENST00000515287 | ENSP00000423504 | 502 |
| | | | ENST00000515440 | ENSP00000422351 | 503 |
| UGP2 | UTP--glucose-1- | ENSG00000169764 | ENST00000467999 | ENSP00000418642 | 504 |
| | uridylyltransferase | | ENST00000496334 | ENSP00000420760 | 505 |
| | phosphate | | | | |
| GLB1 | Beta-galactosidase | ENSG00000170266 | ENST00000307363 | ENSP00000306920 | 506 |
| | | | ENST00000307377 | ENSP00000305920 | 507 |
| | | | ENST00000399402 | ENSP00000382333 | 508 |
| | | | ENST00000415454 | ENSP00000411813 | 509 |
| | | | ENST00000436768 | ENSP00000387989 | 510 |
| | | | ENST00000438227 | ENSP00000401250 | 511 |
| | | | ENST00000440656 | ENSP00000411769 | 512 |
| | | | ENST00000446732 | ENSP00000407365 | 513 |
| | | | ENST00000450835 | ENSP00000403264 | 514 |
| SPATA24 | Spermatogenesis- | ENSG00000170469 | ENST00000514983 | ENSP00000423424 | 515 |
| | associated protein 24 | | | | |
| RBKS | Ribokinase | ENSG00000171174 | ENST00000449378 | ENSP00000413789 | 516 |
| NETO2 | Neuropilin and tolloid- | ENSG00000171208 | ENST00000303155 | ENSP00000306726 | 517 |
| | like protein 2 | | ENST00000562435 | ENSP00000455169 | 518 |
| | | | ENST00000562559 | ENSP00000454213 | 519 |
| | | | ENST00000563078 | ENSP00000456818 | 520 |
| | | | ENST00000564667 | ENSP00000457133 | 521 |
| LRG1 | Leucine-rich alpha-2- | ENSG00000171236 | ENST00000306390 | ENSP00000302621 | 522 |
| | glycoprotein | | | | |
| FAM98B | Protein FAM98B | ENSG00000171262 | ENST00000491535 | ENSP00000453166 | 523 |
| | | | ENST00000559431 | ENSP00000453926 | 524 |
| CHST11 | Carbohydrate | ENSG00000171310 | ENST00000303694 | ENSP00000305725 | 525 |
| | sulfotransferase 11 | | ENST00000546689 | ENSP00000448678 | 526 |
| | | | ENST00000547956 | ENSP00000449093 | 527 |
| | | | ENST00000549260 | ENSP00000450004 | 528 |
| ECEL1 | Endothelin-converting | ENSG00000171551 | ENST00000304546 | ENSP00000302051 | 529 |
| | enzyme-like 1 | | ENST00000409941 | ENSP00000386333 | 530 |
| BCL2L1 | Bcl-2-like protein 1 | ENSG00000171552 | ENST00000307677 | ENSP00000302564 | 531 |
| | | | ENST00000376055 | ENSP00000365223 | 532 |
| | | | ENST00000376062 | ENSP00000365230 | 533 |
| MALT1 | Mucosa-associated | ENSG00000172175 | ENST00000345724 | ENSP00000304161 | 534 |
| | lymphoid tissue | | ENST00000348428 | ENSP00000319279 | 535 |
| | lymphoma translocation | | ENST00000591792 | ENSP00000467222 | 536 |
| | protein 1 | | | | |
| CYP7B1 | 25-hydroxycholesterol 7- | ENSG00000172817 | ENST00000310193 | ENSP00000310721 | 537 |
| | alpha-hydroxylase | | | | |
| HPSE | Heparanase | ENSG00000173083 | ENST00000311412 | ENSP00000308107 | 538 |
| | | | ENST00000405413 | ENSP00000384262 | 539 |
| | | | ENST00000507150 | ENSP00000426139 | 540 |
| | | | ENST00000508891 | ENSP00000421827 | 541 |
| | | | ENST00000509906 | ENSP00000421038 | 542 |
| | | | ENST00000512196 | ENSP00000423265 | 543 |
| | | | ENST00000513463 | ENSP00000421365 | 544 |
| VANGL1 | Vang-like protein 1 | ENSG00000173218 | ENST00000310260 | ENSP00000310800 | 545 |
| | | | ENST00000355485 | ENSP00000347672 | 546 |
| | | | ENST00000369509 | ENSP00000358522 | 547 |
| | | | ENST00000369510 | ENSP00000358523 | 548 |
| CD7 | T-cell antigen CD7 | ENSG00000173762 | ENST00000312648 | ENSP00000312027 | 549 |
| | | | ENST00000578509 | ENSP00000464565 | 550 |
| | | | ENST00000581434 | ENSP00000464546 | 551 |
| | | | ENST00000582480 | ENSP00000464182 | 552 |
| | | | ENST00000583376 | ENSP00000463489 | 553 |
| | | | ENST00000584284 | ENSP00000463612 | 554 |
| HAP1 | Huntingtin-associated | ENSG00000173805 | ENST00000455021 | ENSP00000397242 | 555 |
| | protein 1 | | | | |
| FBXO45 | F-box/SPRY domain- | ENSG00000174013 | ENST00000440469 | ENSP00000389868 | 556 |
| | containing protein 1 | | | | |
| CHST2 | Carbohydrate | ENSG00000175040 | ENST00000309575 | ENSP00000307911 | 557 |
| | sulfotransferase 2 | | | | |
| RM12 | RecQ-mediated genome | ENSG00000175643 | ENST00000572173 | ENSP00000461206 | 558 |
| | instability protein 2 | | | | |
| SLC35E3 | Solute carrier family 35 | ENSG00000175782 | ENST00000398004 | ENSP00000381089 | 559 |
| | member E3 | | ENST00000431174 | ENSP00000403769 | 560 |
| ZBTB38 | Zinc finger and BTB | ENSG00000177311 | ENST00000503809 | ENSP00000422051 | 561 |
| | domain-containing | | | | |
| | protein 38 | | | | |
| YIPF6 | Protein YIPF6 | ENSG00000181704 | ENST00000374622 | ENSP00000363751 | 562 |
| | | | ENST00000451537 | ENSP00000401799 | 563 |
| | | | ENST00000462683 | ENSP00000417573 | 564 |
| CREB3L2 | Cyclic AMP-responsive | ENSG00000182158 | ENST00000330387 | ENSP00000329140 | 565 |
| | element-binding protein | | ENST00000420629 | ENSP00000402889 | 566 |
| | 3-like protein 2 | | ENST00000456390 | ENSP00000403550 | 567 |
| XKRX | XK-related protein 2 | ENSG00000182489 | ENST00000372956 | ENSP00000362047 | 568 |
| | | | ENST00000468904 | ENSP00000419884 | 569 |
| CADM1 | Cell adhesion molecule 1 | ENSG00000182985 | ENST00000331581 | ENSP00000329797 | 570 |
| | | | ENST00000452722 | ENSP00000395359 | 571 |
| | | | ENST00000536727 | ENSP00000440322 | 572 |
| | | | ENST00000537058 | ENSP00000439817 | 573 |
| | | | ENST00000540951 | ENSP00000445375 | 574 |
| | | | ENST00000542447 | ENSP00000439176 | 575 |
| | | | ENST00000542450 | ENSP00000442001 | 576 |
| | | | ENST00000543540 | ENSP00000439847 | 577 |
| | | | ENST00000545380 | ENSP00000442387 | 578 |
| | | | ENST00000612235 | ENSP00000483648 | 579 |
| | | | ENST00000612471 | ENSP00000483793 | 580 |
| | | | ENST00000616271 | ENSP00000484516 | 581 |
| | | | ENST00000621043 | ENSP00000482840 | 582 |
| | | | ENST00000621709 | ENSP00000482924 | 583 |
| LHFP | Lipoma HMGIC fusion | ENSG00000183722 | ENST00000379589 | ENSP00000368908 | 584 |
| | partner | | | | |
| CSF1 | Macrophage colony- | ENSG00000184371 | ENST00000329608 | ENSP00000327513 | 585 |
| | stimulating factor 1 | | ENST00000357302 | ENSP00000349854 | 586 |
| | | | ENST00000369801 | ENSP00000358816 | 587 |
| | | | ENST00000369802 | ENSP00000358817 | 588 |
| | | | ENST00000420111 | ENSP00000407317 | 589 |
| | | | ENST00000488198 | ENSP00000433837 | 590 |
| | | | ENST00000525659 | ENSP00000431547 | 591 |
| | | | ENST00000527192 | ENSP00000434527 | 592 |
| PTP4A3 | Protein tyrosine | ENSG00000184489 | ENST00000329397 | ENSP00000332274 | 593 |
| | phosphatase type IVA 3 | | ENST00000349124 | ENSP00000331730 | 594 |
| | | | ENST00000520105 | ENSP00000428758 | 595 |
| | | | ENST00000521578 | ENSP00000428976 | 596 |
| | | | ENST00000523147 | ENSP00000428725 | 597 |
| | | | ENST00000524028 | ENSP00000430332 | 598 |
| OSBP2 | Oxysterol-binding | ENSG00000184792 | ENST00000445781 | ENSP00000411497 | 599 |
| | protein 2 | | | | |
| METTL7A | Methyltransferase-like | ENSG00000185432 | ENST00000332160 | ENSP00000331787 | 600 |
| | protein 7A | | ENST00000547104 | ENSP00000447542 | 601 |
| | | | ENST00000548553 | ENSP00000448785 | 602 |
| | | | ENST00000550097 | ENSP00000448286 | 603 |
| | | | ENST00000550502 | ENSP00000450239 | 604 |
| TMPRSS6 | Transmembrane | ENSG00000187045 | ENST00000346753 | ENSP00000334962 | 605 |
| | protease serine 6 | | ENST00000381792 | ENSP00000371211 | 606 |
| | | | ENST00000406725 | ENSP00000385453 | 607 |
| | | | ENST00000406856 | ENSP00000384964 | 608 |
| | | | ENST00000423761 | ENSP00000400317 | 609 |
| | | | ENST00000429068 | ENSP00000392433 | 610 |
| | | | ENST00000442782 | ENSP00000397691 | 611 |
| GCNT1 | Beta-1,3-galactosyl-O- | ENSG00000187210 | ENST00000376730 | ENSP00000365920 | 612 |
| | glycosyl-glycoprotein | | ENST00000442371 | ENSP00000415454 | 613 |
| | beta-1,6-N- | | ENST00000444201 | ENSP00000390703 | 614 |
| | acetylglucosaminyltransferase | | | | |
| MAGEH1 | Melanoma-associated | ENSG00000187601 | ENST00000342972 | ENSP00000343706 | 615 |
| | antigen H1 | | | | |
| NEMP2 | Nuclear envelope | ENSG00000189362 | ENST00000343105 | ENSP00000340087 | 616 |
| | integral membrane | | ENST00000409150 | ENSP00000386292 | 617 |
| | protein 2 | | ENST00000414176 | ENSP00000404283 | 618 |
| | | | ENST00000421038 | ENSP00000410306 | 619 |
| | | | ENST00000444545 | ENSP00000403867 | 620 |
| NTNG2 | Netrin-G2 | ENSG00000196358 | ENST00000372179 | ENSP00000361252 | 621 |
| | | | ENST00000393229 | ENSP00000376921 | 622 |
| PDGFA | Platelet-derived growth | ENSG00000197461 | ENST00000354513 | ENSP00000346508 | 623 |
| | factor subunit A | | ENST00000400761 | ENSP00000383572 | 624 |
| | | | ENST00000402802 | ENSP00000383889 | 625 |
| | | | ENST00000405692 | ENSP00000384673 | 626 |
| PDCD1LG2 | Programmed cell death 1 | ENSG00000197646 | ENST00000397747 | ENSP00000380855 | 627 |
| | ligand 2 | | | | |
| TOR4A | Torsin-4A | ENSG00000198113 | ENST00000357503 | ENSP00000350102 | 628 |
| HIBCH | 3-hydroxyisobutyryl-CoA | ENSG00000198130 | ENST00000392333 | ENSP00000376145 | 629 |
| | hydrolase, mitochondrial | | ENST00000414928 | ENSP00000414820 | 630 |
| NTRK1 | High affinity nerve | ENSG00000198400 | ENST00000358660 | ENSP00000351486 | 631 |
| | growth factor receptor | | ENST00000368196 | ENSP00000357179 | 632 |
| | | | ENST00000392302 | ENSP00000376120 | 633 |
| | | | ENST00000497019 | ENSP00000436804 | 634 |
| | | | ENST00000524377 | ENSP00000431418 | 635 |
| FAM19A2 | Protein FAM19A2 | ENSG00000198673 | ENST00000416284 | ENSP00000393987 | 636 |
| | | | ENST00000548780 | ENSP00000449310 | 637 |
| | | | ENST00000549379 | ENSP00000447584 | 638 |
| | | | ENST00000549958 | ENSP00000447280 | 639 |
| | | | ENST00000550003 | ENSP00000449457 | 640 |
| | | | ENST00000551449 | ENSP00000449632 | 641 |
| | | | ENST00000551619 | ENSP00000447305 | 642 |
| | | | ENST00000552075 | ENSP00000449516 | 643 |
| F5 | Coagulation factor V | ENSG00000198734 | ENST00000367796 | ENSP00000356770 | 644 |
| | | | ENST00000367797 | ENSP00000356771 | 645 |
| GK | Glycerol kinase | ENSG00000198814 | ENST00000378943 | ENSP00000368226 | 646 |
| | | | ENST00000427190 | ENSP00000401720 | 647 |
| | | | ENST00000488296 | ENSP00000419771 | 648 |
| INPP5F | Phosphatidylinositide | ENSG00000198825 | ENST00000490818 | ENSP00000487706 | 649 |
| | phosphatase SAC2 | | ENST00000631572 | ENSP00000488726 | 650 |
| CD177 | CD177 antigen | ENSG00000204936 | ENST00000378012 | ENSP00000367251 | 651 |
| | | | ENST00000607855 | ENSP00000483817 | 652 |
| | | | ENST00000618265 | ENSP00000479536 | 653 |
| LEPROT | Leptin Receptor | ENSG00000213625 | ENST00000371065 | ENSP00000360104 | 654 |
| | Overlapping Transcrip | | ENST00000613538 | ENSP00000483521 | 655 |
| TRIM16 | Tripartite motif- | ENSG00000221926 | ENST00000579219 | ENSP00000463639 | 656 |
| | containing protein 16 | | | | |
| LTA | Lymphotoxin-alpha | ENSG00000226979 | ENST00000418386 | ENSP00000413450 | 657 |
| | | | ENST00000454783 | ENSP00000403495 | 658 |
| PROB1 | Proline-rich basic protein 1 | ENSG00000228672 | ENST00000434752 | ENSP00000416033 | 659 |
| SSTR3 | Somatostatin receptor | ENSG00000278195 | ENST00000610913 | ENSP00000480971 | 660 |
| | type 3 | | ENST00000617123 | ENSP00000481325 | 661 |
| CEACAM1 | Carcinoembryonic | ENSG00000079385 | ENST00000161559 | ENSP00000161559 | 662 |
| | antigen-related cell | | ENST00000352591 | ENSP00000244291 | 663 |
| | adhesion molecule 1 | | ENST00000358394 | ENSP00000351165 | 664 |
| | | | ENST00000403444 | ENSP00000384709 | 665 |
| | | | ENST00000403461 | ENSP00000384083 | 666 |
| | | | ENST00000471298 | ENSP00000472633 | 667 |
| | | | ENST00000599389 | ENSP00000471918 | 668 |
| | | | ENST00000600172 | ENSP00000471566 | 669 |
| CTLA4 | Cytotoxic T-lymphocyte | ENSG00000163599 | ENST00000295854 | ENSP00000295854 | 670 |
| | protein 4 | | ENST00000302823 | ENSP00000303939 | 671 |
| | | | ENST00000427473 | ENSP00000409707 | 672 |
| | | | ENST00000472206 | ENSP00000417779 | 673 |
| TIGIT | T-cell immunoreceptor | ENSG00000181847 | ENST00000383671 | ENSP00000373167 | 674 |
| | with Ig and ITIM | | ENST00000461158 | ENSP00000418917 | 675 |
| | domains | | ENST00000481065 | ENSP00000420552 | 676 |
| | | | ENST00000484319 | ENSP00000419706 | 677 |
| | | | ENST00000486257 | ENSP00000419085 | 678 |
| IL2RA | Interleukin-2 receptor | ENSG00000134460 | ENST00000256876 | ENSP00000256876 | 679 |
| | subunit alpha | | ENST00000379954 | ENSP00000369287 | 680 |
| | | | ENST00000379959 | ENSP00000369293 | 681 |
| ENTPD1 | Ectonucleoside | ENSG00000138185 | ENST00000371205 | ENSP00000360248 | 682 |
| | triphosphate | | ENST00000371207 | ENSP00000360250 | 683 |
| | diphosphohydrolase 1 | | ENST00000453258 | ENSP00000390955 | 684 |
| | | | ENST00000483213 | ENSP00000489333 | 685 |
| | | | ENST00000543964 | ENSP00000442968 | 686 |
| | | | ENST00000635076 | ENSP00000489250 | 687 |
| ICOS | Inducible T-cell | ENSG00000163600 | ENST00000316386 | ENSP00000319476 | 688 |
| | costimulator | | ENST00000435193 | ENSP00000415951 | 689 |
| TNFRSF4 | Tumor necrosis factor | ENSG00000186827 | ENST00000379236 | ENSP00000368538 | 690 |
| | receptor superfamily | | | | |
| | member 4 | | | | |
| TNFRSF18 | Tumor necrosis factor | ENSG00000186891 | ENST00000328596 | ENSP00000328207 | 691 |
| | receptor superfamily | | ENST00000379265 | ENSP00000368567 | 692 |
| | member 18 | | ENST00000379268 | ENSP00000368570 | 693 |
| | | | ENST00000486728 | ENSP00000462735 | 694 |
| TNFRSF8 | Tumor necrosis factor | ENSG00000120949 | ENST00000263932 | ENSP00000263932 | 695 |
| | receptor superfamily | | ENST00000417814 | ENSP00000390650 | 696 |
| | member 8 | | ENST00000514649 | ENSP00000421938 | 697 |
| CD274 | Programmed cell death 1 | ENSG00000120217 | ENST00000381573 | ENSP00000370985 | 698 |
| | ligand 1 | | ENST00000381577 | ENSP00000370989 | 699 |
| IL2RB | Interleukin-2 receptor | ENSG00000100385 | ENST00000216223 | ENSP00000216223 | 700 |
| | subunit beta | | ENST00000429622 | ENSP00000402685 | 701 |
| | | | ENST00000445595 | ENSP00000401020 | 702 |
| | | | ENST00000453962 | ENSP00000403731 | 703 |
| TNFRSF9 | Tumor necrosis factor | ENSG00000049249 | ENST00000377507 | ENSP00000366729 | 704 |
| | receptor superfamily | | ENST00000474475 | ENSP00000465272 | 705 |
| | member 9 | | ENST00000615230 | ENSP00000478699 | 706 |
| IKZF2 | Zinc finger protein Helios | ENSG00000030419 | ENST00000442445 | ENSP00000390045 | 707 |

Genes of table VI are characterized by their Ensembl Gene accession number (ENSG), retrievable in the public database EnsEMBL (http://www.ensembl.org). Each related protein isoform is characterized by an Ensembl transcript accession number (ENST) and an Ensembl protein accession number (ENSP).

### Identification of Transcript Isoforms Expressed by Tumor-Treg Cells

An important aspect to be verified in the selection of potential targets of tumor-T reg is that the protein isoforms predicted to be surface exposed/membrane associated by the cell localization algorithms are indeed expressed in tumor Treg cells. Thus, total RNA was extracted from tumor Treg cells isolated from NSCLC or CRC samples and subjected to RT-PCR using specific primer pairs able to discriminate the different isoforms annotated for each gene. Exemplificative results of protein isoforms predicted to be surface exposed and detected in tumor T reg cells is reported in Table VII. Moreover, an example of RT-PCR analysis carried out for SIRPG is reported in FIG. 10.

TABLE VII
Representative examples of transcripts detected
in tumor-infiltrating Treg cells

| GENE | |
|---|---|
| SYMBOL | Surface predicted isoform detected in Tumor Treg cells |
| CCR8 | ENST00000326306 |
| LAYN | ENST00000375614 and/or ENST00000533265 and/or |
| | ENST00000375615 and/or ENST00000525126 |
| CD7 | ENST00000312648 and/or ENST00000584284 |
| CXCL13 | ENST00000286758 |
| FCRL3 | ENST00000492769 and/or ENST00000368184 and/or |
| | ENST00000368186 and/or ENST00000485028 |
| IL1R2 | ENST00000332549 and/or ENST00000393414 |
| IL21R | ENST00000337929 and/or ENST00000395754 and/or |
| | ENST00000564089 |
| NTNG2 | ENST00000393229 |
| SIRPG | ENST00000303415 and/or ENST00000216927 and/or |
| | ENST00000344103 and/or ENST00000381580 and/or |
| | ENST00000381583 |
| TSPAN5 | ENST00000305798 and/or ENST00000505184 |
| TMPRSS3 | ENST00000291532 |
| TMPRSS6 | ENST00000406725 and/or ENST00000406856 |
| NDFIP2 | ENST00000218652 |

### Discussion

Diversity of tumor infiltrating Treg cells should be fully elucidated to understand their functional relevance and prognostic significance in different types of cancer, and to possibly improve the therapeutic efficacy of Treg cell modulation through the selective depletion of tumor infiltrating Treg cells. The transcriptome analysis performed on CRC- and NSCLC-infiltrating T cells showed that tumor-infiltrating Treg cells are different from both circulating and normal tissue-infiltrating Tregs, suggesting that the tumor microenvironment influences specific gene expression in Treg cells. Our findings further support the view that Treg cells from different tissues are instructed by environmental factors to display different gene expression profiles (Panduro et al., 2016). Indeed the list of signature genes includes a number of molecules that are consistently upregulated in tumor infiltrating Treg cells isolated from different tumor types, and these signature genes would have not been identified if the inventors had not profiled specifically tumor infiltrating Treg cells. It was found tumor-infiltrating-Treg signature genes are not only largely shared between CRC and NSCLC infiltrating cells, but are also conserved in breast and gastric cancers as well as in CRC and NSCLC metastatic tumors (in liver and brain respectively) suggesting that expression of these genes is a common feature of tumor infiltrating Treg cells that may correlate with Treg cells specific function within the tumor microenvironment. Although our knowledge on the function of immune checkpoints on lymphocytes is still incomplete, agonist or antagonist monoclonal antibodies targeting checkpoints are in clinical development. Interestingly, it has been found that some of these checkpoints (such as GITR, OX40, TIGIT, LAG-3 and TIM-3) and some of their ligands (such as OX40LG, Galectin-9, CD70) are upregulated also in tumor infiltrating Treg cells, and this fact should be taken into account in interpreting clinical results with checkpoint inhibitors. Indeed, it is likely that assessment of the expression of checkpoints and of their ligands on the various subsets of tumor infiltrating lymphocytes will help to elucidate conflicting results and provide the rationale for combination therapies. Therefore, expression pattern of checkpoints should be evaluated both in tumor infiltrating lymphocytes and in tumor cells. Single-cell analysis on selected tumor Treg signature genes confirmed the whole transcriptomic data and provided information on the expression frequency of these genes. Tumor infiltrating Treg cells express with high frequency genes that are associated with increased suppressor activity, such as the well characterized OX40, CTLA4 and GITR. Moreover, there are a number of interesting and less expected genes the specific expression of which was validated also at the protein level. For example, IL-1 R2 upregulation could be another mechanism that tumor resident Treg cells employ to dampen anti-tumor immune responses through the neutralization of IL-1β function on effector cells. PD-L1 and PD-L2 expression has been recently reported on activated T cells or APCs (Boussiotis et al., 2014; Lesterhuis et al., 2011; Messal et al., 2011) but, to the best of our knowledge, neither PD-L2 nor PD-L1 expression has ever been reported in Treg cells, and our finding that they are overexpressed in tumor infiltrating Treg cells adds an additional level of complexity to the PD1/PD-Ls immunomodulatory axis within the tumor microenvironment. BATF is a transcription factor that has been mainly associated to Th17 development and CD8⁺ T cells differentiation (Murphy et al., 2013). Our findings show that BATF transcript is upregulated in tumor infiltrating Treg cells more than in tumor infiltrating Th17 cells (FIG. 8). Interestingly, expression of BATF in CD8⁺ T cells is induced by IL-21 (Xin et al., 2015), and it was found that IL21R is highly expressed in tumor-infiltrating Treg cells (FIG. 4).

It was showed that tumor infiltrating Treg cells express high amounts of 4-1 BB (CD137) a marker of TcR mediated activation (Schoenbrunn et al., 2012) and have shown they display very high suppressor function on effector T cell proliferation. It could be that expression of the signature genes correlated with the enhanced suppressive ability and so contributed to the establishment of a strong immunosuppressive environment at tumor sites. A corollary to our findings would have that increased number of Treg cells in the tumor environment should associate with a worst clinical outcome. In fact, when LAYN, MAGEH1 and CCR8 (which represent three of the most enriched genes in tumor infiltrating Treg cells) are highly detected in whole tumor samples there is a significant worsening of the 5 years survival of both CRC and NSCLC patients. Although, the functional roles in Treg cells of LAYN, a transmembrane protein with homology to c-type lectin (Borowsky and Hynes, 1998), and of MAGEH1, a member of the Melanoma Antigen Gene family (Weon and Potts, 2015) are unknown, the high expression of the chemokine receptor CCR8 is instead intriguing. Indeed CCL18, the ligand of CCR8 (Islam et al., 2013), is highly expressed in different tumors including NSCLC (Chen et al., 2011; Schutyser et al., 2005). The high specificity of CCR8 expression on tumor infiltrating Treg cells suggests it could be a new interesting therapeutic target to inhibit Treg cells trafficking to tumor sites, without disturbing recruitment of other effector T cells that do not express CCR8. Considerable efforts have been recently put in the development of sophisticated bioinformatics approaches that exploit lymphocyte gene expression data to understand the immune-modulatory networks at tumor sites, to predict clinical responses to immune-therapies, and to define novel therapeutic targets (Bindea et al., 2013a; Bindea et al., 2013b; Gentles et al., 2015). The data here presented represent the first comprehensive RNA-sequencing analysis performed on tumor-infiltrating human CD4⁺ Treg, Th1 and Th17 cells. Our findings highlight the relevance of assessing gene expression patterns of lymphocyte at tumor-sites and suggest that generation of more transcriptomic data of tumor-infiltrating lymphocyte subsets purified from different cancer types may contribute to a better understanding of the dynamics underlying immune modulation in the tumor microenvironment. Moreover, our data represent a resource to generate and validate novel hypotheses that will increase our knowledge on tumor infiltrating Treg cell biology and should lead to the identification of new therapeutic targets.

### REFERENCES

Arpaia, N., Green, J. A., Moltedo, B., Arvey, A., Hemmers, S., Yuan, S., Treuting, P. M., and Rudensky, A. Y. (2015). A Distinct Function of Regulatory T Cells in Tissue Protection. Cell 162,1078-1089.
Bindea, G., Galon, J., and Mlecnik, B. (2013a). CluePedia Cytoscape plugin: pathway insights using integrated experimental and in silico data. Bioinformatics 29, 661-663.
Bindea, G., Mlecnik, B., Tosolini, M., Kirilovsky, A., Waldner, M., Obenauf, A. C., Angell, H., Fredriksen, T., Lafontaine, L., Berger, A., et al. (2013b). Spatiotemporal dynamics of intratumoral immune cells reveal the immune landscape in human cancer. Immunity 39, 782-795.
Bolger, A. M., Lohse, M., and Usadel, B. (2014). Trimmomatic: a flexible trimmer for Illumina sequence data. Bioinformatics 30, 2114-2120.
Borowsky, M. L., and Hynes, R. O. (1998). Layilin, a novel talin-binding transmembrane protein homologous with C-type lectins, is localized in membrane ruffles. J Cell Biol 143, 429-442.
Boussiotis, V. A., Chatterjee, P., and Li, L. (2014). Biochemical signaling of PD-1 on T cells and its functional implications. Cancer J 20, 265-271.
Burzyn, D., Kuswanto, W., Kolodin, D., Shadrach, J. L., Cerletti, M., Jang, Y., Sefik, E., Tan, T. G., Wagers, A. J., Benoist, C., et al. (2013). A special population of regulatory T cells potentiates muscle repair. Cell 155, 1282-1295.
Campbell, D. J., and Koch, M. A. (2011). Phenotypical and functional specialization of FOXP3+ regulatory T cells. Nat Rev Immunol 11, 119-130.
Carthon, B. C., Wolchok, J. D., Yuan, J., Kamat, A., Ng Tang, D. S., Sun, J., Ku, G., Troncoso, P., Logothetis, C. J., Allison, J. P., et al. (2010). Preoperative CTLA-4 blockade: tolerability and immune monitoring in the setting of a presurgical clinical trial. Clin Cancer Res 16, 2861-2871.
Chen, J., Yao, Y., Gong, C., Yu, F., Su, S., Chen, J., Liu, B., Deng, H., Wang, F., Lin, L., et al. (2011). CCL 8 from tumor-associated macrophages promotes breast cancer metastasis via PITPNM3. Cancer Cell 19, 541-555.
Cipolletta, D., Feuerer, M., Li, A., Kamei, N., Lee, J., Shoelson, S. E., Benoist, C., and Mathis, D. (2012). PPAR-gamma is a major driver of the accumulation and phenotype of adipose tissue Treg cells. Nature 486, 549-553.
Duhen, T., Duhen, R., Lanzavecchia, A., Sallusto, F., and Campbell, D. J. (2012). Functionally distinct subsets of human FOXP3+ Treg cells that phenotypically mirror effector Th cells. Blood 119, 4430-4440.
Fridman, W. H., Pages, F., Sautes-Fridman, C., and Galon, J. (2012). The immune contexture in human tumours: impact on clinical outcome. Nat Rev Cancer 12, 298-306.
Galluzzi, L., Buque, A., Kepp, O., Zitvogel, L., and Kroemer, G. (2015). Immunological Effects of Conventional Chemotherapy and Targeted Anticancer Agents. Cancer Cell 28, 690-714.
Geginat, J., Paroni, M., Maglie, S., Alfen, J. S., Kastirr, I., Gruarin, P., De Simone, M., Pagani, M., and Abrignani, S. (2014). Plasticity of human CD4 T cell subsets. Front Immunol 5, 630.
Gentles, A. J., Newman, A. M., Liu, C. L., Bratman, S. V., Feng, W., Kim, D., Nair, V. S., Xu, Y., Khuong, A., Hoang, C. D., et al. (2015). The prognostic landscape of genes and infiltrating immune cells across human cancers. Nat Med 21, 938-945.
Gonzalez-Pons, M., and Cruz-Correa, M. (2015). Colorectal Cancer Biomarkers: Where Are We Now?Biomed Res Int 2015, 149014.
Hodi, F. S., O'Day, S. J., McDermott, D. F., Weber, R. W., Sosman, J. A., Haanen, J. B., Gonzalez, R., Robert, C., Schadendorf, D., Hassel, J. C., et al. (2010). Improved survival with ipilimumab in patients with metastatic melanoma. N Engl J Med 363, 711-723.
Huang da, W., Sherman, B. T., and Lempicki, R. A. (2009). Systematic and integrative analysis of large gene lists using DAVID bioinformatics resources. Nat Protoc 4, 44-57.
Islam, S. A., Ling, M. F., Leung, J., Shreffler, W. G., and Luster, A. D. (2013). Identification of human CCR8 as a CCL18 receptor. J Exp Med 210,1889-1898.
Jacobs, J., Smits, E., Lardon, F., Pauwels, P., and Deschoolmeester, V. (2015). Immune Checkpoint Modulation in Colorectal Cancer: What's New and What to Expect. J Immunol Res 2015, 158038.
Jamal-Hanjani, M., Thanopoulou, E., Peggs, K. S., Quezada, S. A., and Swanton, C. (2013). Tumour heterogeneity and immune-modulation. Curr Opin Pharmacol 13, 497-503.
Joller, N., Lozano, E., Burkett, P. R., Patel, B., Xiao, S., Zhu, C., Xia, J., Tan, T. G., Sefik, E., Yajnik, V., et al. (2014). Treg cells expressing the coinhibitory molecule TIGIT selectively inhibit proinflammatory Th1 and Th17 cell responses. Immunity 40, 569-581.
Josefowicz, S. Z., Lu, L. F., and Rudensky, A. Y. (2012). Regulatory T cells: mechanisms of differentiation and function. Annu Rev Immunol 30, 531-564.
Kharchenko, P. V., Silberstein, L., and Scadden, D. T. (2014). Bayesian approach to single-cell differential expression analysis. Nat Methods 11, 740-742.
Kroemer, G., Galluzzi, L., Zitvogel, L., and Fridman, W. H. (2015). Colorectal cancer: the first neoplasia found to be under immunosurveillance and the last one to respond to immunotherapy?Oncoimmunology 4, e1058597.
Le, D. T., Uram, J. N., Wang, H., Bartlett, B. R., Kemberling, H., Eyring, A. D., Skora, A. D., Luber, B. S., Azad, N. S., Laheru, D., et al. (2015). PD-1 Blockade in Tumors with Mismatch-Repair Deficiency. N Engl J Med 372, 2509-2520.
Lesterhuis, W. J., Steer, H., and Lake, R. A. (2011). PD-L2 is predominantly expressed by Th2 cells. Mol Immunol 49,1-3.
Loffler-Wirth, H., Kalcher, M., and Binder, H. (2015). oposSOM: R-package for high-dimensional portraying of genome-wide expression landscapes on bioconductor. Bioinformatics 31, 3225-3227.
Marabelle, A., Kohrt, H., Sagiv-Barfi, I., Ajami, B., Axtell, R. C., Zhou, G., Rajapaksa, R., Green, M. R., Torchia, J., Brody, J., et al. (2013). Depleting tumor-specific Tregs at a single site eradicates disseminated tumors. J Clin Invest 123, 2447-2463.
Messal, N., Serriari, N. E., Pastor, S., Nunes, J. A., and Olive, D. (2011). PD-L2 is expressed on activated human T cells and regulates their function. Mol Immunol 48, 2214-2219.
Munn, D. H., and Bronte, V. (2015). Immune suppressive mechanisms in the tumor microenvironment. Curr Opin Immunol 39,1-6.
Murphy, T. L., Tussiwand, R., and Murphy, K. M. (2013). Specificity through cooperation: BATF-IRF interactions control immune-regulatory networks. Nat Rev Immunol 13, 499-509.
Nishikawa, H., and Sakaguchi, S. (2010). Regulatory T cells in tumor immunity. Int J Cancer 127, 759-767.
Panduro, M., Benoist, C., and Mathis, D. (2016). Tissue Tregs. Annu Rev Immunol 34, 609-633.
Pardoll, D. M. (2012). The blockade of immune checkpoints in cancer immunotherapy. Nat Rev Cancer 12, 252-264.
Peggs, K. S., Quezada, S. A., Chambers, C. A., Korman, A. J., and Allison, J. P. (2009). Blockade of CTLA-4 on both effector and regulatory T cell compartments contributes to the antitumor activity of anti-CTLA-4 antibodies. J Exp Med 206, 1717-1725.
Ranzani, V., Rossetti, G., Panzeri, I., Arrigoni, A., Bonnal, R. J., Curti, S., Gruarin, P., Provasi, E., Sugliano, E., Marconi, M., et al. (2015). The long intergenic noncoding RNA landscape of human lymphocytes highlights the regulation of T cell differentiation by linc-MAF-4. Nature immunology 16, 318-325.
Ripley, B. D. (1996) Pattern Recognition and Neural Networks. Cambridge: Cambridge University Press. [i, 2, 8, 36, 42, 59, 69, 73, 79, 91, 92]
Sakaguchi, S., Yamaguchi, T., Nomura, T., and Ono, M. (2008). Regulatory T cells and immune tolerance. Cell 133, 775-787.
Sato, M., Larsen, J. E., Lee, W., Sun, H., Shames, D. S., Dalvi, M. P., Ramirez, R. D., Tang, H., DiMaio, J. M., Gao, B., et al. (2013). Human lung epithelial cells progressed to malignancy through specific oncogenic manipulations. Mol Cancer Res 11, 638-650.
Schoenbrunn, A., Frentsch, M., Kohler, S., Keye, J., Dooms, H., Moewes, B., Dong, J., Loddenkemper, C., Sieper, J., Wu, P., et al. (2012). A converse 4-1 BB and CD40 ligand expression pattern delineates activated regulatory T cells (Treg) and conventional T cells enabling direct isolation of alloantigen-reactive natural Foxp3+ Treg. J Immunol 189, 5985-5994.
Schutyser, E., Richmond, A., and Van Damme, J. (2005). Involvement of CC chemokine ligand 18 (CCL18) in normal and pathological processes. J Leukoc Biol 78,14-26.
Selby, M. J., Engelhardt, J. J., Quigley, M., Henning, K. A., Chen, T., Srinivasan, M., and Korman, A. J. (2013). Anti-CTLA-4 antibodies of IgG2a isotype enhance antitumor activity through reduction of intratumoral regulatory T cells. Cancer Immunol Res 1, 32-42.
Sharma, P., and Allison, J. P. (2015). Immune checkpoint targeting in cancer therapy: toward combination strategies with curative potential. Cell 161, 205-214.
Simpson, T. R., Li, F., Montalvo-Ortiz, W., Sepulveda, M. A., Bergerhoff, K., Arce, F., Roddie, C., Henry, J. Y., Yagita, H., Wolchok, J. D., et al. (2013). Fc-dependent depletion of tumor-infiltrating regulatory T cells co-defines the efficacy of anti-CTLA-4 therapy against melanoma. J Exp Med 210, 1695-1710.
Sledzinska, A., Menger, L., Bergerhoff, K., Peggs, K. S., and Quezada, S. A. (2015). Negative immune checkpoints on T lymphocytes and their relevance to cancer immunotherapy. Mol Oncol.
Smith, J. J., Deane, N. G., Wu, F., Merchant, N. B., Zhang, B., Jiang, A., Lu, P., Johnson, J. C., Schmidt, C., Bailey, C. E., et al. (2010). Experimentally derived metastasis gene expression profile predicts recurrence and death in patients with colon cancer. Gastroenterology 138, 958-968.
Sobin, L. H., Gospodarowicz, M. K., Wittekind, C., International Union against Cancer., and ebrary Inc. (2009). TNM classification of malignant tumours (Chichester, West Sussex, UK; Hoboken, NJ: Wiley-Blackwell,), pp. 100-105, 138-146, 7^{th} edition
Teng, M. W., Ngiow, S. F., von Scheidt, B., McLaughlin, N., Sparwasser, T., and Smyth, M. J. (2010). Conditional regulatory T-cell depletion releases adaptive immunity preventing carcinogenesis and suppressing established tumor growth. Cancer Res 70, 7800-7809.
Therneau T. 2013. A package for survival analysis in S. R package version 2.37-4. Topalian, S. L., Drake, C. G., and Pardoll, D. M. (2015). Immune checkpoint blockade: a common denominator approach to cancer therapy. Cancer Cell 27, 450-461.
Torre, L. A., Bray, F., Siegel, R. L., Ferlay, J., Lortet-Tieulent, J., and Jemal, A. (2015). Global cancer statistics, 2012. CA Cancer J Clin 65, 87-108.
Twyman-Saint Victor, C., Rech, A. J., Maity, A., Rengan, R., Pauken, K. E., Stelekati, E., Benci, J. L., Xu, B., Dada, H., Odorizzi, P. M., et al. (2015). Radiation and dual checkpoint blockade activate non-redundant immune mechanisms in cancer. Nature 520, 373-377.
van den Eertwegh, A. J., Versluis, J., van den Berg, H. P., Santegoets, S. J., van Moorselaar, R. J., van der Sluis, T. M., Gall, H. E., Harding, T. C., Jooss, K., Lowy, I., et al. (2012). Combined immunotherapy with granulocyte-macrophage colony-stimulating factor-transduced allogeneic prostate cancer cells and ipilimumab in patients with metastatic castration-resistant prostate cancer: a phase 1 dose-escalation trial. Lancet Oncol 13, 509-517.
Voo, K. S., Bover, L., Harline, M. L., Vien, L. T., Facchinetti, V., Arima, K., Kwak, L. W., and Liu, Y. J. (2013). Antibodies targeting human OX40 expand effector T cells and block inducible and natural regulatory T cell function. J Immunol 191, 3641-3650.
Walter, S., Weinschenk, T., Stenzl, A., Zdrojowy, R., Pluzanska, A., Szczylik, C., Staehler, M., Brugger, W., Dietrich, P. Y., Mendrzyk, R., et al. (2012). Multipeptide immune response to cancer vaccine IMA901 after single-dose cyclophosphamide associates with longer patient survival. Nat Med 18, 1254-1261.
Weon, J. L., and Potts, P. R. (2015). The MAGE protein family and cancer. Curr Opin Cell Biol 37, 1-8.
Wirth, H., von Bergen, M., and Binder, H. (2012). Mining SOM expression portraits: feature selection and integrating concepts of molecular function. BioData Min 5, 18.
Xin, G., Schauder, D. M., Lainez, B., Weinstein, J. S., Dai, Z., Chen, Y., Esplugues, E., Wen, R., Wang, D., Parish, I. A., et al. (2015). A Critical Role of IL-21-Induced BATF in Sustaining CD8-T-Cell-Mediated Chronic Viral Control. Cell Rep 13,1118-1124.
Yang, J. C., Hughes, M., Kammula, U., Royal, R., Sherry, R. M., Topalian, S. L., Suri, K. B., Levy, C., Allen, T., Mavroukakis, S., et al. (2007). Ipilimumab (anti-CTLA4 antibody) causes regression of metastatic renal cell cancer associated with enteritis and hypophysitis. J Immunother 30, 825-830.
Zitvogel, L., Galluzzi, L., Smyth, M. J., and Kroemer, G. (2013). Mechanism of action of conventional and targeted anticancer therapies: reinstating immunosurveillance. Immunity 39, 74-88.
Sebastian Briesemeister, Jörg RahnenfOhrer, and Oliver Kohlbacher, (2010). Going from where to why—interpretable prediction of protein subcellular localization, Bioinformatics, 26(9):1232-1238.
Lukas Käll, Anders Krogh and Erik L. L. Sonnhammer. Advantages of combined transmembrane topology and signal peptide prediction—the Phobius web server. Nucleic Acids Res., 35:W429-32, July 2007

1.-25. (canceled)

26. A method for identifying an antibody acting as an anti-tumoral agent that depletes tumor-infiltrating regulatory T cells, comprising the steps of:
a) assaying candidate antibodies for their binding specificity to CCR8;
b) selecting antibodies having a specific binding activity to CCR8;
c) testing the specific binding antibodies in a cell system comprising tumor infiltrating regulatory T cells for their capacity of inhibiting proliferation and/or inducing an apoptotic response of the tumor infiltrating regulatory T cells, thereby identifying an antibody that depletes tumor-infiltrating regulatory T cells.

27. The method of claim 26, wherein the depletion of tumor-infiltrating regulatory T cells comprises inducing antibody-dependent cell-mediated cytotoxicity (ADCC).

28. An in vitro method for monitoring the efficacy of a therapeutic treatment in a subject of a solid tumor which is a non-small cell lung cancer or a metastasis derived therefrom, said method comprising the steps of:
a) obtaining an isolated biological sample containing tumor infiltrating T regulatory cells from the subject;
b) detecting CCR8 in said biological sample;
c) comparing the detected CCR8 to a control selected from a biological sample obtained from the same subject before initiation of the therapeutic treatment or taken at a time during the course of the therapeutic treatment, wherein a lower amount of CCR8 in the biological sample than in the control indicates effective treatment of the tumor.


SEQUENCE LISTING (791 sequences)

SEQ ID NO: 1 - protein, 374 aa, Homo sapiens
MRPGTALQAVLLAVLLVGLRAATGRLLSGQPVCRGGTQRPCYKVIYFHDTSRRLNFEEAKEACRRDGGQLVSIESEDEQKLIEKFIENLLPSDGDFWIGLRRREEKQSNSTACQDLYAWTDGSISQFRNWYVDEPSCGSEVCVVMYHQPSAPAGIGGPYMFQWNDDRCNMKNNFICKYSDEKPAVPSREAEGEETELTTPVLPEETQEEDAKKTFKESREAALNLAYILIPSIPLLLLLVVTTVVCWVWICRKRKREQPDPSTKKQHTIWPSPHQGNSPDLEVYNVIRKQSEADLAETRPDLKNISFRVCSGEATPDDMSCDYDNMAVNPSESGFVTLVSVESGFVTNDIYEFSPDQMGRSKESGWVENEIYGY

SEQ ID NO: 2 - protein, 382 aa, Homo sapiens
MRPGTALQAVLLAVLLVGLRAATGRLLSASDLDLRGGQPVCRGGTQRPCYKVIYFHDTSRRLNFEEAKEACRRDGGQLVSIESEDEQKLIEKFIENLLPSDGDFWIGLRRREEKQSNSTACQDLYAWTDGSISQFRNWYVDEPSCGSEVCVVMYHQPSAPAGIGGPYMFQWNDDRCNMKNNFICKYSDEKPAVPSREAEGEETELTTPVLPEETQEEDAKKTFKESREAALNLAYILIPSIPLLLLLVVTTVVCWVWICRKRKREQPDPSTKKQHTIWPSPHQGNSPDLEVYNVIRKQSEADLAETRPDLKNISFRVCSGEATPDDMSCDYDNMAVNPSESGFVTLVSVESGFVTNDIYEFSPDQMGRSKESGWVENEIYGY

SEQ ID NO: 3 - protein, 229 aa, Homo sapiens
MVTSGLGSGGVRRNKAIAQPARTFMLGLMAAYHNLEKPAVPSREAEGEETELTTPVLPEETQEEDAKKTFKESREAALNLAYILIPSIPLLLLLVVTTVVCWVWICRKRKREQPDPSTKKQHTIWPSPHQGNSPDLEVYNVIRKQSEADLAETRPDLKNISFRVCSGEATPDDMSCDYDNMAVNPSESGFVTLVSVESGFVTNDIYEFSPDQMGRSKESGWVENEIYGY

SEQ ID NO: 4 - protein, 270 aa, Homo sapiens
MRPGTALQAVLLAVLLVGLRAATGRLLSASDLDLRGGQPVCRGGTQRPCYKVIYFHDTSRRLNFEEAKEACRRDGGQLVSIESEDEQKLIEKFIENLLPSDGDFWIGLRRREEKQSNSTACQDLYAWTDGSISQFRNWYVDEPSCGSEVCVVMYHQPSAPAGIGGPYMFQWNDDRCNMKNNFICKYSDEKPAVPSREAEGEETELTTPVLPEETQEEDAKKTFKESREAALNLAYILIPSIPLLLLLVVTTVVCWVWICRKRQKTGAARP

SEQ ID NO: 5 - protein, 134 aa, Homo sapiens
MRPGTALQAVLLAVLLVGLRAATGRLLSGQPVCRGGTQRPCYKVIYFHDTSRRLNFEEAKEACRRDGGQLVSIESEDEQKLIEKFIENLLPSDGDFWIGLRRREEKQSNSTACQDLYAWTDGSISQFRETSSSF

SEQ ID NO: 6 - protein, 108 aa, Homo sapiens
MVTSGLGSGGVRRNKAIAQPARTFMLGLMAAYHNLEKPAVPSREAEGEETELTTPVLPEETQEEDAKKTFKESREAALNLAYILIPSIPLLLLLVVTTVVCWVWICRK

SEQ ID NO: 7 - protein, 109 aa, Homo sapiens
MYHQPSAPAGIGGPYMFQWNDDRCNMKNNFICKYSDEKPAVPSREAEGEETELTTPVLPEETQEEDAKKTFKESREAALNLAYILIPSIPLLLLLVVTTVVCWVWICRK

SEQ ID NO: 8 - protein, 262 aa, Homo sapiens
MRPGTALQAVLLAVLLVGLRAATGRLLSGQPVCRGGTQRPCYKVIYFHDTSRRLNFEEAKEACRRDGGQLVSIESEDEQKLIEKFIENLLPSDGDFWIGLRRREEKQSNSTACQDLYAWTDGSISQFRNWYVDEPSCGSEVCVVMYHQPSAPAGIGGPYMFQWNDDRCNMKNNFICKYSDEKPAVPSREAEGEETELTTPVLPEETQEEDAKKTFKESREAALNLAYILIPSIPLLLLLVVTTVVCWVWICRKRQKTGAARP

SEQ ID NO: 9 - protein, 49 aa, Homo sapiens
MYHQPSAPAGIGGPYMFQWNDDRCNMKNNFICKYSDEKPAVPSREAEGE

SEQ ID NO: 10 - protein, 355 aa, Homo sapiens
MDYTLDLSVTTVTDYYYPDIFSSPCDAELIQTNGKLLLAVFYCLLFVFSLLGNSLVILVLVVCKKLRSITDVYLLNLALSDLLFVFSFPFQTYYLLDQWVFGTVMCKVVSGFYYIGFYSSMFFITLMSVDRYLAVVHAVYALKVRTIRMGTTLCLAVWLTAIMATIPLLVFYQVASEDGVLQCYSFYNQQTLKWKIFTNFKMNILGLLIPFTIFMFCYIKILHQLKRCQNHNKTKAIRLVLIVVIASLLFWVPFNVVLFLTSLHSMHILDGCSISQQLTYATHVTEIISFTHCCVNPVIYAFVGEKFKKHLSEIFQKSCSQIFNYLGRQMPRESCEKSSSCQQHSSRSSSVDYIL

SEQ ID NO: 11 - protein, 118 aa, Homo sapiens
MDYTLDLSVTTVTDYYYPDIFSSPCDAELIQTNDLLSAGPVGVWDCNVQSGVWLLLHWLLQQHVFHHPHECGQVPGCCPCRVCPKGEDDQDGHNAVPGSMANRHYGYHPIASVLPSGL

SEQ ID NO: 12 - protein, 538 aa, Homo sapiens
MPRGWAAPLLLLLLQGGWGCPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKEGWNPHLLLLLLLVIVFIPAFWSLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 13 - protein, 538 aa, Homo sapiens; identical to SEQ ID NO: 12

SEQ ID NO: 14 - protein, 538 aa, Homo sapiens; identical to SEQ ID NO: 12

SEQ ID NO: 15 - protein, 467 aa, Homo sapiens
MRPQELPRLAFPLLLLLLLLLPPPPCPAHSATRFDPTWESLDARQLPAWFDQAKFGIFIHWGVFSVPSFGSEWFWWYWQKEKIPKYVEFMKDNYPPSFKYEDFGPLFTAKFFNANQWADIFQASGAKYIVLTSKHHEGFTLWGSEYSWNWNAIDEGPKRDIVKELEVAIRNRTDLRFGLYYSLFEWFHPLFLEDESSSFHKRQFPVSKTLPELYELVNNYQPEVLWSDGDGGAPDQYWNSTGFLAWLYNESPVRGTVVTNDRWGAGSICKHGGFYTCSDRYNPGHLLPHKWENCMTIDKLSWGYRREAGISDYLTIEELVKQLVETVSCGGNLLMNIGPTLDGTISVVFEERLRQMGSWLKVNGEAIYETHTWRSQNDTVTPDVWYTSKPKEKLVYAIFLKWPTSGQLFLGHPKAILGATEVKLLGHGQPLNWISLEQNGIMVELPQLTIHQMPCKWGWALALTNVI

SEQ ID NO: 16 - protein, 109 aa, Homo sapiens
QLVETVSCGGNLLMNIGPTLDGTISVVFEERLRQMGSWLKVNGEAIYETHTWRSQNDTVTPDVWYTSKPKEKLVYAIFLKWPTSGQLFLGHPKAILGATEVRGNFTLFL

SEQ ID NO: 17 - protein, 350 aa, Homo sapiens
MSGHKCSYPWDLQDRYAQDKSVVNKMQQKYWETKQAFIKATGKKEDEHVVASDADLDAKLELFHSIQRTCLDLSKAIVLYQKRICFLSQEENELGKFLRSQGFQDKTRAGKMMQATGKALCFSSQQRLALRNPLCRFHQEVETFRHRAISDTWLTVNRMEQCRTEYRGALLWMKDVSQELDPDLYKQMEKFRKVQTQVRLAKKNFDKLKMDVCQKVDLLGASRCNLLSHMLATYQTTLLHFWEKTSHTMAAIHESFKGYQPYEFTTLKVWKLEGPLENLEKSKVLFISQAKSIAESIIIPGLRMLKRELCPQQSLFLSFFLSFFFFFFFFTASGFNTMRIYCNPKARIEP

SEQ ID NO: 18 - protein, 443 aa, Homo sapiens
MAASPHTLSSRLLTGCVGGSVWYLERRTIQDSPHKFLHLLRNVNKQWITFQHFSFLKRMYVTQLNRSHNQQVRPKPEPVASPFLEKTSSGQAKAEIYEMRPLSPPSLSLSRKPNEKELIELEPDSVIEDSIDVGKETKEEKRWKEMKLQVYDLPGILARLSKIKLTALVVSTTAAGFALAPGPFDWPCFLLTSVGTGLASCAANSINQFFEVPFDSNMNRTKNRPLVRGQISPLLAVSFATCCAVPGVAILTLGVNPLTGALGLFNIFLYTCCYTPLKRISIANTWVGAVVGAIPPVMGWTAATGSLDAGAFLLGGILYSWQFPHFNALSWGLREDYSRGGYCMMSVTHPGLCRRVALRHCLALLVLSAAAPVLDITTWTFPIMALPINAYISYLGFRFYVDADRRSSRRLFFCSLWHLPLLLLLMLTCKRPSGGGDAGPPPS

SEQ ID NO: 19 - protein, 168 aa, Homo sapiens
MCFPKVLSDDMKKLKARMVMSSLAELEDDFKEGYLETVAAYYEEQHPELTPLLEKERDGLRCRGNRSPVPDVEDPATEEPGESFCDKVMRWFQAMLQRLQTWWHGVLAWVKEKVVALVHAVQALWKQFQSFCCSLSELFMSSFQSYGAPRGDKEELTPQKCSEPQSSK

SEQ ID NO: 20 - protein, 234 aa, Homo sapiens
MCFPKVLSDDMKKLKARMVMLLPTSAQGLGAWVSACDTEDTVGHLGPWRDKDPALWCQLCLSSQHQAIERFYDKMQNAESGRGQVMSSLAELEDDFKEGYLETVAAYYEEQHPELTPLLEKERDGLRCRGNRSPVPDVEDPATEEPGESFCDKVMRWFQAMLQRLQTWWHGVLAWVKEKVVALVHAVQALWKQFQSFCCSLSELFMSSFQSYGAPRGDKEELTPQKCSEPQSSK

SEQ ID NO: 21 - protein, 131 aa, Homo sapiens
MCFPKVLSDDMKKLKARMDDFKEGYLETVAAYYEEQHPELTPLLEKERDGLRCRGNRSPVPDVEDPATEEPGESFCDKVMRWFQAMLQRLQTWWHGVLAWVKEKVVALVHAVQALWKQFQSFCCSLSELFM

SEQ ID NO: 22 - protein, 442 aa, Homo sapiens
MCFPKVLSDDMKKLKARMVMLLPTSAQGLGAWVSACDTEDTVGHLGPWRDKDPALWCQLCLSSQHQAIERFYDKMQNAESGRGQVMSSLAELEDDFKEGYLETVAAYYEEQHPELTPLLEKERDGLRCRGNRSPVPDVEDPATEEPGESFCDKVMRWFQAMLQRLQTWWHGVLAWVKEKVVALVHAVQALWKQFQSFCCSLSELFMSSFQSYGATRGARRSMCFPKVLSDDMKKLKARMVMLLPTSAQGLGAWVSACDTEDTVGHLGPWRDKDPALWCQLCLSSQHQAIERFYDKMQNAESGRGQVMSSLAELEDDFKEGYLETVAAYYEEQHPELTPLLEKERDGLRCRGNRSPVPDVEDPATEEPGESFCDKVMRWFQAMLQRLQTWWHGVLAWVKEKVVALVHAVQALWKQFQSFCCSLSELFMSSFQSYGATRGARRS

SEQ ID NO: 23 - protein, 168 aa, Homo sapiens; identical to SEQ ID NO: 19

SEQ ID NO: 24 - protein, 234 aa, Homo sapiens; identical to SEQ ID NO: 20

SEQ ID NO: 25 - protein, 148 aa, Homo sapiens
MCFPKVLSDDMKKLKARMVMSSLAELEELTPLLEKERDGLRCRGNRSPVPDVEDPATEEPGESFCDKVMRWFQAMLQRLQTWWHGVLAWVKEKVVALVHAVQALWKQFQSFCCSLSELFMSSFQSYGAPRGDKEELTPQKCSEPQSSK

SEQ ID NO: 26 - protein, 234 aa, Homo sapiens; identical to SEQ ID NO: 20

SEQ ID NO: 27 - protein, 225 aa, Homo sapiens
MCFPKVLSDDMKKLKARMVMLLPTSAQGLGAWVSACDTEDTVGHLGPWRDKDPALWCQLCLSSQHQAIERFYDKMQNAESGRGQVMSSLAELEDDFKEGYLETVAAYYEEQHPELTPLLEKERDGLRCRGNRSPVPDVEDPATEEPGESFCDKVMRWFQAMLQRLQTWWHGVLAWVKEKVVALVHAVQALWKQFQSFCCSLSELFMSSFQSYGAPRGDKEELTPQ

SEQ ID NO: 28 - protein, 225 aa, Homo sapiens
MCFPKVLSDDMKKLKARMVMLLPTSAQGLGAWVSACDTEDTVGHLGPWRDKDPALWCQLCLSSQHQAIERFYDKMQNAESGRGQDDFKEGYLETVAAYYEEQHPELTPLLEKERDGLRCRGNRSPVPDVEDPATEEPGESFCDKVMRWFQAMLQRLQTWWHGVLAWVKEKVVALVHAVQALWKQFQSFCCSLSELFMSSFQSYGAPRGDKEELTPQKCSEPQSSK

SEQ ID NO: 29 - protein, 168 aa, Homo sapiens; identical to SEQ ID NO: 19

SEQ ID NO: 30 - protein, 212 aa, Homo sapiens
MCFPKGLGAWVSACDTEDTVGHLGPWRDKDPALWCQLCLSSQHQAIERFYDKMQNAESGRGQVMSSLAELEDDFKEGYLETVAAYYEEQHPELTPLLEKERDGLRCRGNRSPVPDVEDPATEEPGESFCDKVMRWFQAMLQRLQTWWHGVLAWVKEKVVALVHAVQALWKQFQSFCCSLSELFMSSFQSYGAPRGDKEELTPQKCSEPQSSK

SEQ ID NO: 31 - protein, 317 aa, Homo sapiens
MQEGELAISPISPVAAMPPLGTHVQARCEAQINLLGEGGICKLPGRLRIQPALWSREDVLHWLRWAEQEYSLPCTAEHGFEMNGRALCILTKDDFRHRAPSSGDVLYELLQYIKTQRRALVCGPFFGGIFRLKTPTQHSPVPPEEVTGPSQMDTRRGHLLQPPDPGLTSNFGHLDDPGLARWTPGKEESLNLCHCAELGCRTQGVCSFPAMPQAPIDGRIADCRLLWDYVYQLLLDTRYEPYIKWEDKDAKIFRVVDPNGLARLWGNHKNRVNMTYEKMSRALRHYYKLNIIKKEPGQKLLFRNGLQLIFIFIWSFQ

SEQ ID NO: 32 - protein, 262 aa, Homo sapiens
MQEGELAISPISPVAAMPPLGTHVQARCEAQINLLGEGGICKLPGRLRDVLYELLQYIKTQRRALVCGPFFGGIFRLKTPTQHSPVPPEEVTGPSQMDTRRGHLLQPPDPGLTSNFGHLDDPGLARWTPGKEESLNLCHCAELGCRTQGVCSFPAMPQAPIDGRIADCRLLWDYVYQLLLDTRYEPYIKWEDKDAKIFRVVDPNGLARLWGNHKNRVNMTYEKMSRALRHYYKLNIIKKEPGQKLLFRNGLQLIFIFIWSFQ

SEQ ID NO: 33 - protein, 888 aa, Homo sapiens
MKVARFQKIPNGENETMIPVLTSKKASELPVSEVASILQADLQNGLNKCEVSHRRAFHGWNEFDISEDEPLWKKYISQFKNPLIMLLLASAVISVLMHQFDDAVSITVAILIVVTVAFVQEYRSEKSLEELSKLVPPECHCVREGKLEHTLARDLVPGDTVCLSVGDRVPADLRLFEAVDLSIDESSLTGETTPCSKVTAPQPAATNGDLASRSNIAFMGTLVRCGKAKGVVIGTGENSEFGEVFKMMQAEEAPKTPLQKSMDLLGKQLSFYSFGIIGIIMLVGWLLGKDILEMFTISVSLAVAAIPEGLPIVVTVTLALGVMRMVKKRAIVKKLPIVETLGCCNVICSDKTGTLTKNEMTVTHIFTSDGLHAEVTGVGYNQFGEVIVDGDVVHGFYNPAVSRIVEAGCVCNDAVIRNNTLMGKPTEGALIALAMKMGLDGLQQDYIRKAEYPFSSEQKWMAVKCVHRTQQDRPEICFMKGAYEQVIKYCTTYQSKGQTLTLTQQQRDVYQQEKARMGSAGLRVLALASGPELGQLTFLGLVGIIDPPRTGVKEAVTTLIASGVSIKMITGDSQETAVAIASRLGLYSKTSQSVSGEEIDAMDVQQLSQIVPKVAVFYRASPRHKMKIIKSLQKNGSVVAMTGDGVNDAVALKAADIGVAMGQTGTDVCKEAADMILVDDDFQTIMSAIEEGKGIYNNIKNFVRFQLSTSIAALTLISLATLMNFPNPLNAMQILWINIIMDGPPAQSLGVEPVDKDVIRKPPRNWKDSILTKNLILKILVSSIIIVCGTLFVFWRELRDNVITPRDTTMTFTCFVFFDMFNALSSRSQTKSVFEIGLCSNRMFCYAVLGSIMGQLLVIYFPPLQKVFQTESLSILGLALGEEWTAAG

SEQ ID NO: 34 - protein, 949 aa, Homo sapiens
MKVARFQKIPNGENETMIPVLTSKKASELPVSEVASILQADLQNGLNKCEVSHRRAFHGWNEFDISEDEPLWKKYISQFKNPLIMLLLASAVISVLMHQFDDAVSITVAILIVVTVAFVQEYRSEKSLEELSKLVPPECHCVREGKLEHTLARDLVPGDTVCLSVGDRVPADLRLFEAVDLSIDESSLTGETTPCSKVTAPQPAATNGDLASRSNIAFMGTLVRCGKAKGVVIGTGENSEFGEVFKMMQAEEAPKTPLQKSMDLLGKQLSFYSFGIIGIIMLVGWLLGKDILEMFTISVSLAVAAIPEGLPIVVTVTLALGVMRMVKKRAIVKKLPIVETLGCCNVICSDKTGTLTKNEMTVTHIFTSDGLHAEVTGVGYNQFGEVIVDGDVVHGFYNPAVSRIVEAGCVCNDAVIRNNTLMGKPTEGALIALAMKMGLDGLQQDYIRKAEYPFSSEQKWMAVKCVHRTQQDRPEICFMKGAYEQVIKYCTTYQSKGQTLTLTQQQRDVYQQEKARMGSAGLRVLALASGPELGQLTFLGLVGIIDPPRTGVKEAVTTLIASGVSIKMITGDSQETAVAIASRLGLYSKTSQSVSGEEIDAMDVQQLSQIVPKVAVFYRASPRHKMKIIKSLQKNGSVVAMTGDGVNDAVALKAADIGVAMGQTGTDVCKEAADMILVDDDFQTIMSAIEEGKGIYNNIKNFVRFQLSTSIAALTLISLATLMNFPNPLNAMQILWINIIMDGPPAQSLGVEPVDKDVIRKPPRNWKDSILTKNLILKILVSSIIIVCGTLFVFWRELRDNVITPRDTTMTFTCFVFFDMFNALSSRSQTKSVFEIGLCSNRMFCYAVLGSIMGQLLVIYFPPLQKVFQTESLSILDLLFLLGLTSSVCIVAEIIKKVERSREKIQKHVSSTSSSFLEVWLWERSGQQLVEIHPHLETGLPLTEDVSCV

SEQ ID NO: 35 - protein, 939 aa, Homo sapiens
MKVARFQKIPNGENETMIPVLTSKKASELPVSEVASILQADLQNGLNKCEVSHRRAFHGWNEFDISEDEPLWKKYISQFKNPLIMLLLASAVISVLMHQFDDAVSITVAILIVVTVAFVQEYRSEKSLEELSKLVPPECHCVREGKLEHTLARDLVPGDTVCLSVGDRVPADLRLFEAVDLSIDESSLTGETTPCSKVTAPQPAATNGDLASRSNIAFMGTLVRCGKAKGVVIGTGENSEFGEVFKMMQAEEAPKTPLQKSMDLLGKQLSFYSFGIIGIIMLVGWLLGKDILEMFTISVSLAVAAIPEGLPIVVTVTLALGVMRMVKKRAIVKKLPIVETLGCCNVICSDKTGTLTKNEMTVTHIFTSDGLHAEVTGVGYNQFGEVIVDGDVVHGFYNPAVSRIVEAGCVCNDAVIRNNTLMGKPTEGALIALAMKMGLDGLQQDYIRKAEYPFSSEQKWMAVKCVHRTQQDRPEICFMKGAYEQVIKYCTTYQSKGQTLTLTQQQRDVYQQEKARMGSAGLRVLALASGPELGQLTFLGLVGIIDPPRTGVKEAVTTLIASGVSIKMITGDSQETAVAIASRLGLYSKTSQSVSGEEIDAMDVQQLSQIVPKVAVFYRASPRHKMKIIKSLQKNGSVVAMTGDGVNDAVALKAADIGVAMGQTGTDVCKEAADMILVDDDFQTIMSAIEEGKGIYNNIKNFVRFQLSTSIAALTLISLATLMNFPNPLNAMQILWINIIMDGPPAQSLGVEPVDKDVIRKPPRNWKDSILTKNLILKILVSSIIIVCGTLFVFWRELRDNVITPRDTTMTFTCFVFFDMFNALSSRSQTKSVFEIGLCSNRMFCYAVLGSIMGQLLVIYFPPLQKVFQTESLSILDLLFLLGLTSSVCIVAEIIKKVERSREKIQKHVWLWERSGQQLVEIHPHLETGLPLTEDVSCV

SEQ ID NO: 36 - protein, 919 aa, Homo sapiens
MKVARFQKIPNGENETMIPVLTSKKASELPVSEVASILQADLQNGLNKCEVSHRRAFHGWNEFDISEDEPLWKKYISQFKNPLIMLLLASAVISVLMHQFDDAVSITVAILIVVTVAFVQEYRSEKSLEELSKLVPPECHCVREGKLEHTLARDLVPGDTVCLSVGDRVPADLRLFEAVDLSIDESSLTGETTPCSKVTAPQPAATNGDLASRSNIAFMGTLVRCGKAKGVVIGTGENSEFGEVFKMMQAEEAPKTPLQKSMDLLGKQLSFYSFGIIGIIMLVGWLLGKDILEMFTISVSLAVAAIPEGLPIVVTVTLALGVMRMVKKRAIVKKLPIVETLGCCNVICSDKTGTLTKNEMTVTHIFTSDGLHAEVTGVGYNQFGEVIVDGDVVHGFYNPAVSRIVEAGCVCNDAVIRNNTLMGKPTEGALIALAMKMGLDGLQQDYIRKAEYPFSSEQKWMAVKCVHRTQQDRPEICFMKGAYEQVIKYCTTYQSKGQTLTLTQQQRDVYQQEKARMGSAGLRVLALASGPELGQLTFLGLVGIIDPPRTGVKEAVTTLIASGVSIKMITGDSQETAVAIASRLGLYSKTSQSVSGEEIDAMDVQQLSQIVPKVAVFYRASPRHKMKIIKSLQKNGSVVAMTGDGVNDAVALKAADIGVAMGQTGTDVCKEAADMILVDDDFQTIMSAIEEGKGIYNNIKNFVRFQLSTSIAALTLISLATLMNFPNPLNAMQILWINIIMDGPPAQSLGVEPVDKDVIRKPPRNWKDSILTKNLILKILVSSIIIVCGTLFVFWRELRDNVITPRDTTMTFTCFVFFDMFNALSSRSQTKSVFEIGLCSNRMFCYAVLGSIMGQLLVIYFPPLQKVFQTESLSILDLLFLLGLTSSVCIVAEIIKKVERSREKIQKHVSSTSSSFLEV

SEQ ID NO: 37 - protein, 944 aa, Homo sapiens
MDSLLPPSRFSYFKKYPLHAIRRYLSTLRNQRAEEQVARFQKIPNGENETMIPVLTSKKASELPVSEVASILQFKNPLIMLLLASAVISVLMHQFDDAVSITVAILIVVTVAFVQEYRSEKSLEELSKLVPPECHCVREGKLEHTLARDLVPGDTVCLSVGDRVPADLRLFEAVDLSIDESSLTGETTPCSKVTAPQPAATNGDLASRSNIAFMGTLVRCGKAKGVVIGTGENSEFGEVFKMMQAEEAPKTPLQKSMDLLGKQLSFYSFGIIGIIMLVGWLLGKDILEMFTISVSLAVAAIPEGLPIVVTVTLALGVMRMVKKRAIVKKLPIVETLGCCNVICSDKTGTLTKNEMTVTHIFTSDGLHAEVTGVGYNQFGEVIVDGDVVHGFYNPAVSRIVEAGCVCNDAVIRNNTLMGKPTEGALIALAMKMGLDGLQQDYIRKAEYPFSSEQKWMAVKCVHRTQQDRPEICFMKGAYEQVIKYCTTYQSKGQTLTLTQQQRDVYQQEKARMGSAGLRVLALASGPELGQLTFLGLVGIIDPPRTGVKEAVTTLIASGVSIKMITGDSQETAVAIASRLGLYSKTSQSVSGEEIDAMDVQQLSQIVPKVAVFYRASPRHKMKIIKSLQKNGSVVAMTGDGVNDAVALKAADIGVAMGQTGTDVCKEAADMILVDDDFQTIMSAIEEGKGIYNNIKNFVRFQLSTSIAALTLISLATLMNFPNPLNAMQILWINIIMDGPPAQSLGVEPVDKDVIRKPPRNWKDSILTKNLILKILVSSIIIVCGTLFVFWRELRDNVITPRDTTMTFTCFVFFDMFNALSSRSQTKSVFEIGLCSNRMFCYAVLGSIMGQLLVIYFPPLQKVFQTESLSILDLLFLLGLTSSVCIVAEIIKKVERSREKIQKHVSSTSSSFLEVWLWERSGQQLVEIHPHLETGLPLTEDVSCV

SEQ ID NO: 38 - protein, 215 aa, Homo sapiens
VTVTLALGVMRMVKKRAIVKKLPIVETLGCCNVICSDKTGTLTKNEMTVTHIFTSDGLHAEVTGVGYNQFGEVIVDGDVVHGFYNPAVSRIVEAGCVCNDAVIRNNTLMGKPTEGALIALAMKMGLDGLQQDYIRKAEYPFSSEQKWMAVKCVHRTQQDRPEICFMKGAYEQVIKYCTTYQSKGQTLTLTQQQRDVYQQEKARMGSAGLRVYLVF

SEQ ID NO: 39 - protein, 903 aa, Homo sapiens
XKCEVSHRRAFHGWNEFDISEDEPLWKKYISQFKNPLIMLLLASAVISVLMHQFDDAVSITVAILIVVTVAFVQEYRSEKSLEELSKLVPPECHCVREGKLEHTLARDLVPGDTVCLSVGDRVPADLRLFEAVDLSIDESSLTGETTPCSKVTAPQPAATNGDLASRSNIAFMGTLVRCGKAKGVVIGTGENSEFGEVFKMMQAEEAPKTPLQKSMDLLGKQLSFYSFGIIGIIMLVGWLLGKDILEMFTISVSLAVAAIPEGLPIVVTVTLALGVMRMVKKRAIVKKLPIVETLGCCNVICSDKTGTLTKNEMTVTHIFTSDGLHAEVTGVGYNQFGEVIVDGDVVHGFYNPAVSRIVEAGCVCNDAVIRNNTLMGKPTEGALIALAMKMGLDGLQQDYIRKAEYPFSSEQKWMAVKCVHRTQQDRPEICFMKGAYEQVIKYCTTYQSKGQTLTLTQQQRDVYQQEKARMGSAGLRVLALASGPELGQLTFLGLVGIIDPPRTGVKEAVTTLIASGVSIKMITGDSQETAVAIASRLGLYSKTSQSVSGEEIDAMDVQQLSQIVPKVAVFYRASPRHKMKIIKSLQKNGSVVAMTGDGVNDAVALKAADIGVAMGQTGTDVCKEAADMILVDDDFQTIMSAIEEGKGIYNNIKNFVRFQLSTSIAALTLISLATLMNFPNPLNAMQILWINIIMDGPPAQSLGVEPVDKDVIRKPPRNWKDSILTKNLILKILVSSIIIVCGTLFVFWRELRDNVITPRDTTMTFTCFVFFDMFNALSSRSQTKSVFEIGLCSNRMFCYAVLGSIMGQLLVIYFPPLQKVFQTESLSILDLLFLLGLTSSVCIVAEIIKKVERSREKIQKHVSSTSSSFLEVWLWERSGQQLVEIHPHLETGLPLTEDVSCV

SEQ ID NO: 40 - protein, 903 aa, Homo sapiens
MIPVLTSKKASELPVSEVASILQADLQNGLNKCEVSHRRAFHGWNEFDISEDEPLWKKYISQFKNPLIMLLLASAVISVLMHQFDDAVSITVAILIVVTVAFVQEYRSEKSLEELSKLVPPECHCVREGKLEHTLARDLVPGDTVCLSVGDRVPADLRLFEAVDLSIDESSLTGETTPCSKVTAPQPAATNGDLASRSNIAFMGTLVRCGKAKGVVIGTGENSEFGEVFKMMQAEEAPKTPLQKSMDLLGKQLSFYSFGIIGIIMLVGWLLGKDILEMFTISVSLAVAAIPEGLPIVVTVTLALGVMRMVKKRAIVKKLPIVETLGCCNVICSDKTGTLTKNEMTVTHIFTSDGLHAEVTGVGYNQFGEVIVDGDVVHGFYNPAVSRIVEAGCVCNDAVIRNNTLMGKPTEGALIALAMKMGLDGLQQDYIRKAEYPFSSEQKWMAVKCVHRTQQDRPEICFMKGAYEQVIKYCTTYQSKGQTLTLTQQQRDVYQQEKARMGSAGLRVLALASGPELGQLTFLGLVGIIDPPRTGVKEAVTTLIASGVSIKMITGDSQETAVAIASRLGLYSKTSQSVSGEEIDAMDVQQLSQIVPKVAVFYRASPRHKMKIIKSLQKNGSVVAMTGDGVNDAVALKAADIGVAMGQTGTDVCKEAADMILVDDDFQTIMSAIEEGKGIYNNIKNFVRFQLSTSIAALTLISLATLMNFPNPLNAMQILWINIIMDGPPAQSLGVEPVDKDVIRKPPRNWKDSILTKNLILKILVSSIIIVCGTLFVFWRELRDNVITPRDTTMTFTCFVFFDMFNALSSRSQTKSVFEIGLCSNRMFCYAVLGSIMGQLLVIYFPPLQKVFQTESLSILDLLFLLGLTSSVCIVAEIIKKVERSREKIQKHVSSTSSSFLEV

SEQ ID NO: 41 - protein, 120 aa, Homo sapiens
MKVARFQKIPNGENETMIPVLTSKKASELPVSEVASILQADLQNGLNKCEVSHRRAFHGWNEFDISEDEPLWKKYISQFKNPLIMLLLASAVISVLMHQFDDAVSITVAILIVVTVAFVQ

SEQ ID NO: 42 - protein, 953 aa, Homo sapiens
MDSLLPPSRFSYFKKYPLHAIRRYLSTLRNQRAEEQVARFQKIPNGENETMIPVLTSKKASELPVSEVASILQADLQNGLNKCEVSHRRAFHGWNEFDISEDEPLWKKYISQFKNPLIMLLLASAVISVLMHQFDDAVSITVAILIVVTVAFVQEYRSEKSLEELSKLVPPECHCVREGKLEHTLARDLVPGDTVCLSVGDRVPADLRLFEAVDLSIDESSLTGETTPCSKVTAPQPAATNGDLASRSNIAFMGTLVRCGKAKGVVIGTGENSEFGEVFKMMQAEEAPKTPLQKSMDLLGKQLSFYSFGIIGIIMLVGWLLGKDILEMFTISVSLAVAAIPEGLPIVVTVTLALGVMRMVKKRAIVKKLPIVETLGCCNVICSDKTGTLTKNEMTVTHIFTSDGLHAEVTGVGYNQFGEVIVDGDVVHGFYNPAVSRIVEAGCVCNDAVIRNNTLMGKPTEGALIALAMKMGLDGLQQDYIRKAEYPFSSEQKWMAVKCVHRTQQDRPEICFMKGAYEQVIKYCTTYQSKGQTLTLTQQQRDVYQQEKARMGSAGLRVLALASGPELGQLTFLGLVGIIDPPRTGVKEAVTTLIASGVSIKMITGDSQETAVAIASRLGLYSKTSQSVSGEEIDAMDVQQLSQIVPKVAVFYRASPRHKMKIIKSLQKNGSVVAMTGDGVNDAVALKAADIGVAMGQTGTDVCKEAADMILVDDDFQTIMSAIEEGKGIYNNIKNFVRFQLSTSIAALTLISLATLMNFPNPLNAMQILWINIIMDGPPAQSLGVEPVDKDVIRKPPRNWKDSILTKNLILKILVSSIIIVCGTLFVFWRELRDNVITPRDTTMTFTCFVFFDMFNALSSRSQTKSVFEIGLCSNRMFCYAVLGSIMGQLLVIYFPPLQKVFQTESLSILDLLFLLGLTSSVCIVAEIIKKVERSREKIQKHVSSTSSSFLEV

SEQ ID NO: 43 - protein, 50 aa, Homo sapiens
LVDDDFQTIMSAIEEGKGIYNNIKNFVRFQLSTFCGSILLWMDPQLRALE

SEQ ID NO: 44 - protein, 973 aa, Homo sapiens
MDSLLPPSRFSYFKKYPLHAIRRYLSTLRNQRAEEQVARFQKIPNGENETMIPVLTSKKASELPVSEVASILQADLQNGLNKCEVSHRRAFHGWNEFDISEDEPLWKKYISQFKNPLIMLLLASAVISVLMHQFDDAVSITVAILIVVTVAFVQEYRSEKSLEELSKLVPPECHCVREGKLEHTLARDLVPGDTVCLSVGDRVPADLRLFEAVDLSIDESSLTGETTPCSKVTAPQPAATNGDLASRSNIAFMGTLVRCGKAKGVVIGTGENSEFGEVFKMMQAEEAPKTPLQKSMDLLGKQLSFYSFGIIGIIMLVGWLLGKDILEMFTISVSLAVAAIPEGLPIVVTVTLALGVMRMVKKRAIVKKLPIVETLGCCNVICSDKTGTLTKNEMTVTHIFTSDGLHAEVTGVGYNQFGEVIVDGDVVHGFYNPAVSRIVEAGCVCNDAVIRNNTLMGKPTEGALIALAMKMGLDGLQQDYIRKAEYPFSSEQKWMAVKCVHRTQQDRPEICFMKGAYEQVIKYCTTYQSKGQTLTLTQQQRDVYQQEKARMGSAGLRVLALASGPELGQLTFLGLVGIIDPPRTGVKEAVTTLIASGVSIKMITGDSQETAVAIASRLGLYSKTSQSVSGEEIDAMDVQQLSQIVPKVAVFYRASPRHKMKIIKSLQKNGSVVAMTGDGVNDAVALKAADIGVAMGQTGTDVCKEAADMILVDDDFQTIMSAIEEGKGIYNNIKNFVRFQLSTSIAALTLISLATLMNFPNPLNAMQILWINIIMDGPPAQSLGVEPVDKDVIRKPPRNWKDSILTKNLILKILVSSIIIVCGTLFVFWRELRDNVITPRDTTMTFTCFVFFDMFNALSSRSQTKSVFEIGLCSNRMFCYAVLGSIMGQLLVIYFPPLQKVFQTESLSILDLLFLLGLTSSVCIVAEIIKKVERSREKIQKHVWLWERSGQQLVEIHPHLETGLPLTEDVSCV

SEQ ID NO: 45 - protein, 82 aa, Homo sapiens
MIPVLTSKKASELPVSEVASILQADLQNGLNKCEVSHRRAFHGWNEFDISEDEPLWKKYISQFKNPLIMLLLASAVISVLMH

SEQ ID NO: 46 - protein, 919 aa, Homo sapiens; identical to SEQ ID NO: 36

SEQ ID NO: 47 - protein, 314 aa, Homo sapiens
XEGALIALAMKMGLDGLQQDYIRKAEYPFSSEQKWMAVKCVHRTQQDRPEICFMKGAYEQVIKYCTTYQSKGQTLTLTQQQRDVYQQEKARMGSAGLRASRLGLYSKTSQSVSGEEIDAMDVQQLSQIVPKVAVFYRASPRHKMKIIKSLQKNGSVVAMTGDGVNDAVALKAADIGVAMGQTGTDVCKEAADMILVDDDFQTIMSAIEEGKGIYNNIKNFVRFQLSTSIAALTLISLATLMNFPNPLNAMQILWINIIMDGPPAQSLGVEPVDKDVIRKPPRNWKDSILTKNLILKILVSSIIIVCGTLFVFWR

SEQ ID NO: 48 - protein, 102 aa, Homo sapiens
MKVARFQKIPNGENETMIPVLTSKKASELPVSEVASILQADLQNGLNKCEVSHRRAFHGWNEFDISEDEPLWKKYISQFKNPLIMLLLASAVISVLMHQFDD

SEQ ID NO: 49 - protein, 919 aa, Homo sapiens; identical to SEQ ID NO: 36

SEQ ID NO: 50 - protein, 923 aa, Homo sapiens
MIPVLTSKKASELPVSEVASILQADLQNGLNKCEVSHRRAFHGWNEFDISEDEPLWKKYISQFKNPLIMLLLASAVISVLMHQFDDAVSITVAILIVVTVAFVQEYRSEKSLEELSKLVPPECHCVREGKLEHTLARDLVPGDTVCLSVGDRVPADLRLFEAVDLSIDESSLTGETTPCSKVTAPQPAATNGDLASRSNIAFMGTLVRCGKAKGVVIGTGENSEFGEVFKMMQAEEAPKTPLQKSMDLLGKQLSFYSFGIIGIIMLVGWLLGKDILEMFTISVSLAVAAIPEGLPIVVTVTLALGVMRMVKKRAIVKKLPIVETLGCCNVICSDKTGTLTKNEMTVTHIFTSDGLHAEVTGVGYNQFGEVIVDGDVVHGFYNPAVSRIVEAGCVCNDAVIRNNTLMGKPTEGALIALAMKMGLDGLQQDYIRKAEYPFSSEQKWMAVKCVHRTQQDRPEICFMKGAYEQVIKYCTTYQSKGQTLTLTQQQRDVYQQEKARMGSAGLRVLALASGPELGQLTFLGLVGIIDPPRTGVKEAVTTLIASGVSIKMITGDSQETAVAIASRLGLYSKTSQSVSGEEIDAMDVQQLSQIVPKVAVFYRASPRHKMKIIKSLQKNGSVVAMTGDGVNDAVALKAADIGVAMGQTGTDVCKEAADMILVDDDFQTIMSAIEEGKGIYNNIKNFVRFQLSTSIAALTLISLATLMNFPNPLNAMQILWINIIMDGPPAQSLGVEPVDKDVIRKPPRNWKDSILTKNLILKILVSSIIIVCGTLFVFWRELRDNVITPRDTTMTFTCFVFFDMFNALSSRSQTKSVFEIGLCSNRMFCYAVLGSIMGQLLVIYFPPLQKVFQTESLSILDLLFLLGLTSSVCIVAEIIKKVERSREKIQKHVWLWERSGQQLVEIHPHLETGLPLTEDVSCV

SEQ ID NO: 51 - protein, 131 aa, Homo sapiens
MDLLGKQLSFYSFGIIGIIMLVGWLLGKDILEMFTISVSLAVAAIPEGLPIVVTVTLALGVMRMVKKRAIVKKLPIVETLGCCNVICSDKTGTLTKNEMTVTHIFTSDGLHAEVTGVGYNQFGEVIVDGDV

SEQ ID NO: 52 - protein, 888 aa, Homo sapiens; identical to SEQ ID NO: 33

SEQ ID NO: 53 - protein, 233 aa, Homo sapiens [sequence omitted]

SEQ ID NO: 54 - protein, 220 aa, Homo sapiens
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPMLT

SEQ ID NO: 55 - protein, 335 aa, Homo sapiens
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 56 - protein, 314 aa, Homo sapiens
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEVKRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 57 - protein, 86 aa, Homo sapiens
MLGIWTLLPLVLTSVARLSSKSVNAQVTDINSKGLELRKTVTTVETQNLEGLHHDGQFCHKPCPPDVNMESSRNAHSPATPSAKRK

SEQ ID NO: 58 - protein, 103 aa, Homo sapiens
MLGIWTLLPLVLTSVARLSSKSVNAQVTDINSKGLELRKTVTTVETQNLEGLHHDGQFCHKPCPPDVNMESSRNAHSPATPSAKRKDPDLTWGGFVFFFCQFH

SEQ ID NO: 59 - protein, 149 aa, Homo sapiens
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHDVNMESSRNAHSPATPSAKRKDPDLTWGGFVFFFCQFH

SEQ ID NO: 60 - protein, 132 aa, Homo sapiens
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHDVNMESSRNAHSPATPSAKRK

SEQ ID NO: 61 - protein, 149 aa, Homo sapiens; identical to SEQ ID NO: 59

SEQ ID NO: 62 - protein, 244 aa, Homo sapiens
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPMESCSIAQAGVQGCNHGSLQPQSPGLK

SEQ ID NO: 63 - protein, 373 aa, Homo sapiens
MLRSVWNFLKRHKKKCIFLGTVLGGVYILGKYGQKKIREIQEREAAEYIAQARRQYHFESNQRTCNMTVLSMLPTLREALMQQLNSESLTALLKNRPSNKLEIWEDLKIISFTRSTVAVYSTCMLVVLLRVQLNIIGGYIYLDNAAVGKNGTTILAPPDVQQQYLSSIQHLLGDGLTELITVIKQAVQKVLGSVSLKHSLSLLDLEQKLKEIRNLVEQHKSSSWINKDGSKPLLCHYMMPDEETPLAVQACGLSPRDITTIKLLNETRDMLESPDFSTVLNTCLNRGFSRLLDNMAEFFRPTEQDLQHGNSMNSLSSVSLPLAKIIPIVNGQIHSVCSETPSHFVQDLLTMEQVKDFAANVYEAFSTPQQLEK

SEQ ID NO: 64 - protein, 182 aa, Homo sapiens
MLRSVWNFLKRHKKKCIFLGTVLGVLSMLPTLREALMQQLNSESLTALLKNRPSNKLEIWEDLKIISFTRSTVAVYSTCMLVVLLRVQLNIIGGYIYLDNAAVGKNGTTILAPPDVQQQYLSSIQHLLGDGLTELITVIKQAVQKVLGSVSLKHSLSLLDLEQKLKEIRNLVEQHKSSSWIN

SEQ ID NO: 65 - protein, 188 aa, Homo sapiens
MPGKHQHFQEPEVGCCGKYFLFGFNIVFWFSVFLGLIFFLELATGILAFVFKDWIRDQLNLFINNNVKAYRDDIDLQNLIDFAQEYEDVLNTQCGYDVRLKLELEQQGFIHTKGCVGQFEKWLQDNLIVVAGVFMGIALLQVPLWPHVPLPLPGPPSLSPHLSSVLQIFGICLAQNLVSDIKAVKANW

SEQ ID NO: 66 - protein, 332 aa, Homo sapiens
MPGKHQHFQEPEVGCCGKYFLFGFNIVFWVLGALFLAIGLWAWGEKGVLSNISALTDLGGLDPVWLFVVVGGVMSVLGFAGCIGALRENTFLLKFFSVFLGLIFFLELATGILAFVFKDWIRDQLNLFINNNVKAYRDDIDLQNLIDFAQEYWSCCGARGPNDWNLNIYFNCTDLNPSRERCGVPFSCCVRDPAEDVLNTQCGYDVRLKLVRGELEQQGFIHTKGCVGQFEKWLQDNLIVVAGVFMGIALLQIFGICLAQNLVSDIKAVKANWSKWNDDFENHWLTPTISEVLSTAGPQQNSLTGAPGPAPPSRHVFFGLGGLYPEPTFKNW

SEQ ID NO: 67 - protein, 58 aa, Homo sapiens
MPGKHQHFQEPEVGCCGKYFLFGFNIVFWVSAGCWEPCSWLSASGPGVRRAFSRTSQR

SEQ ID NO: 68 - protein, 309 aa, Homo sapiens
MPGKHQHFQEPEVGCCGKYFLFGFNIVFWVLGALFLAIGLWAWGEKGVLSNISALTDLGGLDPVWLFVVVGGVMSVLGFAGCIGALRENTFLLKFDWIRDQLNLFINNNVKAYRDDIDLQNLIDFAQEYWSCCGARGPNDWNLNIYFNCTDLNPSRERCGVPFSCCVRDPAEDVLNTQCGYDVRLKLVRGELEQQGFIHTKGCVGQFEKWLQDNLIVVAGVFMGIALLQIFGICLAQNLVSDIKAVKANWSKWNDDFENHWLTPTISEVLSTAGPQQNSLTGAPGPAPPSRHVFFGLGGLYPEPTFKNW

SEQ ID NO: 69 - protein, 257 aa, Homo sapiens
MPGKHQHFQEPEVGCCGKYFLFGFNIVFWVLGALFLAIGLWAWGEKGVLSNISALTDLGGLDPVWLFVVVGGVMSVLGFAGCIGALRENTFLLKFLATGILAFVFKDWIRDQLNLFINNNVKAYRDDIDLQNLIDFAQEYWSCCGARGPNDWNLNIYFNCTDLNPSRERCGVPFSCCVRDPAEDVLNTQCGYDVRLKLELEQQGFIHTKGCVGQFEKWLQDNLIVVAGVFMGIALLQIFGICLAQNLVSDIKAVKAN

SEQ ID NO: 70 - protein, 203 aa, Homo sapiens
XGKHQHFQEPEVGCCGKYFLFGFNIVFWFSVFLGLIFFLELATGILAFVFKDWIRDQLNLFINNNVKAYRDDIDLQNLIDFAQEYWSCCGARGPNDWNLNIYFNCTDLNPSRERCGVPFSCCVRDPAEDVLNTQCGYDVRLKLELEQQGFIHTKGCVGQFEKWLQDNLIVVAGVFMGIALLQIFGICLAQNLVSDIKAVKANW

SEQ ID NO: 71 - protein, 329 aa, Homo sapiens
MPGKHQHFQEPEVGCCGKYFLFGFNIVFWVLGALFLAIGLWAWGEKGVLSNISALTDLGGLDPVWLFVVVGGVMSVLGFAGCIGALRENTFLLKFFSVFLGLIFFLELATGILAFVFKDWIRDQLNLFINNNVKAYRDDIDLQNLIDFAQEYWSCCGARGPNDWNLNIYFNCTDLNPSRERCGVPFSCCVRDPAEDVLNTQCGYDVRLKLELEQQGFIHTKGCVGQFEKWLQDNLIVVAGVFMGIALLQIFGICLAQNLVSDIKAVKANWSKWNDDFENHWLTPTISEVLSTAGPQQNSLTGAPGPAPPSRHVFFGLGGLYPEPTFKNW

SEQ ID NO: 72 - protein, 263 aa, Homo sapiens
MPGKHQHFQEPEVGCCGKYFLFGFNIVFWVLGALFLAIGLWAWGEKGVLSNISALTDLGGLDPVWLFVVVGGVMSVLGFAGCIGALRENTFLLKFFSVFLGLIFFLELATGILAFVFKDWIRDQLNLFINNNVKAYRDDIDLQNLIDFAQEYWSCCGARGPNDWNLNIYFNCTDLNPSRERCGVPFSCCVRDPAEDVLNTQCGYDVRLKLELEQQGFIHTKGCVGQFEKWLQDNLIVVAGVFMGIALLQIFGICLAQNLEQME

SEQ ID NO: 73 - protein, 83 aa, Homo sapiens
MAAATASPRSLLVLLQVVVLALAQIRGPPGERGPPGPPGPPGVPGSDGIDGPPGPVGLPGEIGIRGPKGDPGPDGPSGPPGPP

SEQ ID NO: 74 - protein, 689 aa, Homo sapiens
MAAATASPRSLLVLLQVVVLALAQIRGPPGERGPPGPPGPPGVPGSDGIDGDNGPPGKAGPPGPKGEPGKAGPDGPDGKPGIDGLTGAKGEPGPMGIPGVKGQPGLPGPPGLPGPGFAGPPGPPGPVGLPGEIGIRGPKGDPGPDGPSGPPGPPGKPGRPGTIQGLEGSADFLCPTNCPPGMKGPPGLQGVKGHAGKRGILGDPGHQGKPGPKGDVGASGEQGIPGPPGPQGIRGYPGMAGPKGETGPHGYKGMVGAIGATGPPGEEGPRGPPGRAGEKGDEGSPGIRGPQGITGPKGATGPPGINGKDGTPGTPGMKGSAGQAGQPGSPGHQGLAGVPGQPGTKGGPGDQGEPGPQGLPGFSGPPGKEGEPGPRGEIGPQGIMGQKGDQGERGPVGQPGPQGRQGPKGEQGPPGIPGPQGLPGVKGDKGSPGKTGPRGKVGDPGVAGLPGEKGEKGESGEPGPKGQQGVRGEPGYPGPSGDAGAPGVQGYPGPPGPRGLAGNRGVPGQPGRQGVEGRDATDQHIVDVALKMLQEQLAEVAVSAKREALGAVGMMGPPGPPGPPGYPGKQGPHGHPGPRGVPGIVGAVGQIGNTGPKGKRGEKGDPGEVGRGHPGMPGPPGIPGLPGRPGQAINGKDGDRGSPGAPGEAGRPGLPGPVGLPGFCEPAACLGASAYASARLTEPGSIKGP

SEQ ID NO: 75 - protein, 226 aa, Homo sapiens
XSPFRGPPGAAGARRGPPGERGPPGPPGPPGVPGSDGIDGDNGPPGKAGPPGPKGEPGKAGPDGPDGKPGIDGLTGAKGEPGPMGIPGVKGQPGLPGPPGLPGPGFAGPPGPPGPVGLPGEIGIRGPKGDPGPDGPSGPPGPPGKPGRPGTIQGLEGSADFLCPTNCPPGMKGPPGLQGVKGHAGKRGILGDPGHQGKPGPKGDVGASGEQGIPGPPGPQGIRGYP

SEQ ID NO: 76 - protein, 694 aa, Homo sapiens
MKHLKRWWSAGGGLLHLTLLLSLAGLRVDLDLYLLLPPPTLLQDELLFLGGPASSAYALSPFSASGGWGRAGHLHPKGRELDPAAPPEGQLLREVRALGVPFVPRTSVDAWLVHSVAAGSADEAHGLLGAAAASSTGGAGASVDGGSQAVQGGGGDPRAARSGPLDAGEEEKAPAEPTAQVPDAGGCASEENGVLREKHEAVDHSSQHEENEERVSAQKENSLQQNDDDENKIAEKPDWEAEKTTESRNERHLNGTDTSFSLEDLFQLLSSQPENSLEGISLGDIPLPGSISDGMNSSAHYHVNFSQAISQDVNLHEAILLCPNNTFRRDPTARTSQSQEPFLQLNSHTTNPEQTLPGTNLTGFLSPVDNHMRNLTSQDLLYDLDINIFDEINLMSLATEDNFDPIDVSQLFDEPDSDSGLSLDSSHNNTSVIKSNSSHSVCDEGAIGYCTDHESSSHHDLEGAVGGYYPEPSKLCHLDQSDSDFHGDLTFQHVFHNHTYHLQPTAPESTSEPFPWPGKSQKIRSRYLEDTDRNLSRDEQRAKALHIPFSVDEIVGMPVDSFNSMLSRYYLTDLQVSLIRDIRRRGKNKVAAQNCRKRKLDIILNLEDDVCNLQAKKETLKREQAQCNKAINIMKQKLHDLYHDIFSRLRDDQGRPVNPNHYALQCTHDGSILIVPKELVASGHKKETQKGKRK

SEQ ID NO: 77 - protein, 86 aa, Homo sapiens
CNCGLVFHLQDPWVPTGPGAVQKQREHPPDYQWYALDQLPPDVQHKANESAILSTACFYGLVRPPRSGLYKLSYNQNPTSILIKYT

SEQ ID NO: 78 - protein, 1722 aa, Homo sapiens
MRTGWATPRRPAGLLMLLFWFFDLAEPSGRAANDPFTIVHGNTGKCIKPVYGWIVADDCDETEDKLWKWVSQHRLFHLHSQKCLGLDITKSVNELRMFSCDSSAMLWWKCEHHSLYGAARYRLALKDGHGTAISNASDVWKKGGSEESLCDQPYHEIYTRDGNSYGRPCEFPFLIDGTWHHDCILDEDHSGPWCATTLNYEYDRKWGICLKPENGCEDNWEKNEQFGSCYQFNTQTALSWKEAYVSCQNQGADLLSINSAAELTYLKEKEGIAKIFWIGLNQLYSARGWEWSDHKPLNFLNWDPDRPSAPTIGGSSCARMDAESGLWQSFSCEAQLPYVCRKPLNNTVELTDVWTYSDTRCDAGWLPNNGFCYLLVNESNSWDKAHAKCKAFSSDLISIHSLADVEVVVTKLHNEDIKEEVWIGLKNINIPTLFQWSDGTEVTLTYWDENEPNVPYNKTPNCVSYLGELGQWKVQSCEEKLKYVCKRKGEKLNDASSDKMCPPDEGWKRHGETCYKIYEDEVPFGTNCNLTITSRFEQEYLNDLMKKYDKSLRKYFWTGLRDVDSCGEYNWATVGGRRRAVTFSNWNFLEPASPGGCVAMSTGKSVGKWEVKDCRSFKALSICKKMSGPLGPEEASPKPDDPCPEGWQSFPASLSCYKVFHAERIVRKRNWEEAERFCQALGAHLSSFSHVDEIKEFLHFLTDQFSGQHWLWIGLNKRSPDLQGSWQWSDRTPVSTIIMPNEFQQDYDIRDCAAVKVFHRPWRRGWHFYDDREFIYLRPFACDTKLEWVCQIPKGRTPKTPDWYNPDRAGIHGPPLIIEGSEYWFVADLHLNYEEAVLYCASNHSFLATITSFVGLKAIKNKIANISGDGQKWWIRISEWPIDDHFTYSRYPWHRFPVTFGEECLYMSAKTWLIDLGKPTDCSTKLPFICEKYNVSSLEKYSPDSAAKVQCSEQWIPFQNKCFLKIKPVSLTFSQASDTCHSYGGTLPSVLSQIEQDFITSLLPDMEATLWIGLRWTAYEKINKWTDNRELTYSNFHPLLVSGRLRIPENFFEEESRYHCALILNLQKSPFTGTWNFTSCSERHFVSLCQKYSEVKSRQTLQNASETVKYLNNLYKIIPKTLTWHSAKRECLKSNMQLVSITDPYQQAFLSVQALLHNSSLWIGLFSQDDELNFGWSDGKRLHFSRWAETNGQLEDCVVLDTDGFWKTVDCNDNQPGAICYYSGNETEKEVKPVDSVKCPSPVLNTPWIPFQNCCYNFIITKNRHMATTQDEVHTKCQKLNPKSHILSIRDEKENNFVLEQLLYFNYMASWVMLGITYRNKSLMWFDKTPLSYTHWRAGRPTIKNEKFLAGLSTDGFWDIQTFKVIEEAVYFHQHSILACKIEMVDYKEEYNTTLPQFMPYEDGIYSVIQKKVTWYEALNMCSQSGGHLASVHNQNGQLFLEDIVKRDGFPLWVGLSSHDGSESSFEWSDGSTFDYIPWKGQTSPGNCVLLDPKGTWKHEKCNSVKDGAICYKPTKSKKLSRLTYSSRCPAAKENGSRWIQYKGHCYKSDQALHSFSEAKKLCSKHDHSATIVSIKDEDENKFVSRLMRENNNITMRVWLGLSQHSVDQSWSWLDGSEVTFVKWENKSKSGVGRCSMLIASNETWKKVECEHGFGRVVCKVPLGPDYTAIAIIVATLSILVLMGGLIWFLFQRHRLHLAGFSSVRYAQGVNEDEIMLPSFHD

SEQ ID NO: 79 - protein, 306 aa, Homo sapiens
MAAVDDLQFEEFGNAATSLTANPDATTVNIEDPGETPKHQPGSPRGSGREEDDELLGNDDSDKTELLAGQKKSSPFWTFEYYQTFFDVDTYQVFDRIKGSLLPIPGKNFVRLYIRSNPDLYGPFWICATLVFAIAISGNLSNFLIHLGEKTYHYVPEFRKVSIAATIIYAYAWLVPLALWGFLMWRNSKVMNIVSYSFLEIVCVYGYSLFIYIPTAILWIIPQKAVRWILVMIALGISGSLLAMTFWPAVREDNRRVALATIVTIVLLHMLLSVGCLAYFFDAPEMDHLPTTTATPNQTVAAAKSS

SEQ ID NO: 80 - protein, 123 aa, Homo sapiens
MWRNSKVMNIVSYSFLEIVCVYGYSLFIYIPTAILWIIPQKAVRWILVMIALGISGSLLAMTFWPAVREDNRRVALATIVTIVLLHMLLSVGCLAYFFDAPEMDHLPTTTATPNQTVAAAKSS

SEQ ID NO: 81 - protein, 217 aa, Homo sapiens
MAAVDDLQFEEFGNAATSLTANPDATTVNIEDPGETPKHQPGSPRGSGREEDDELLGNDDSDKTELLAGQKKSSPFWTFEYYQTFFDVDTYQVFDRIKGSLLPIPGKNFVRLYIRSNPDLYGPFWICATLVFAIAISGNLSNFLIHLGEKTYHYVPEFRKVSIAATIIYAYAWLVPLALWGFLMWRNSKVMNIVSYSFLEIVCVYGYSLFIYIPTAV

SEQ ID NO: 82 - protein, 306 aa, Homo sapiens; identical to SEQ ID NO: 79

SEQ ID NO: 83 - protein, 298 aa, Homo sapiens
MAAAEPAVLALPNSGAGGAGAPSGTVPVLFCFSVFARPSSVPHGAGYELLIQKFLSLYGDQIDMHRKFVVQLFAEEWGQYVDLPKGFAVSERCKVRLVPLQIQLTTLGNLTPSSTVFFCCDMQERFRPAIKYFGDIISVGQRLLQGARILGIPVIVTEQYPKGLGSTVQEIDLTGVKLVLPKTKFSMVLPEVEAALAEIPGVRSVVLFGVETHVCIQQTALELVGRGVEVHIVADATSSRSMMDRMFALERLARTGIIVTTSEAVLLQLVADKDHPKFKEIQNLIKASAPESGLLSKV

SEQ ID NO: 84 - protein, 188 aa, Homo sapiens
MAAAEPAVLALPNSGAGGAGAPSGTVPVLFCFSVFARPSSVPHGAGYELLIQKFLSLYGDQIDMHRKFVVQLFAEEWGQYVDLPKGFAVSERCKLTTLGNLTPSSTVFFCCDMQERFRPAIKYFGDIISVGQRLLQGARILGIPVIVTEQYPKGLGSTVQEIDLTGVKLVLPKTKFSMVLPEVEAALA

SEQ ID NO: 85 - protein, 711 aa, Homo sapiens
MKLKLNVLTIILLPVHLLITIYSALIFIPWYFLTNAKKKNAMAKRIKAKPTSDKPGSPYRSVTHFDSLAVIDIPGADTLDKLFDHAVSKFGKKDSLGTREILSEENEMQPNGKVFKKLILGNYKWMNYLEVNRRVNNFGSGLTALGLKPKNTIAIFCETRAEWMIAAQTCFKYNFPLVTLYATLGKEAVVHGLNESEASYLITSVELLESKLKTALLDISCVKHIIYVDNKAINKAEYPEGFEIHSMQSVEELGSNPENLGIPPSRPTPSDMAIVMYTSGSTGRPKGVMMHHSNLIAGMTGQCERIPGLGPKDTYIGYLPLAHVLELTAEISCFTYGCRIGYSSPLTLSDQSSKIKKGSKGDCTVLKPTLMAAVPEIMDRIYKNVMSKVQEMNYIQKTLFKIGYDYKLEQIKKGYDAPLCNLLLFKKVKALLGGNVRMMLSGGAPLSPQTHRFMNVCFCCPIGQGYGLTESCGAGTVTEVTDYTTGRVGAPLICCEIKLKDWQEGGYTINDKPNPRGEIVIGGQNISMGYFKNEEKTAEDYSVDENGQRWFCTGDIGEFHPDGCLQIIDRKKDLVKLQAGEYVSLGKVEAALKNCPLIDNICAFAKSDQSYVISFVVPNQKRLTLLAQQKGVEGTWVDICNNPAMEAEILKEIREAANAMKLERFEIPIKVRLSPEPWTPETGLVTDAFKLKRKELRNHYLKDIERMYGGK

SEQ ID NO: 86 - protein, 711 aa, Homo sapiens; identical to SEQ ID NO: 85

SEQ ID NO: 87 - protein, 54 aa, Homo sapiens
MKLKLNVLTIILLPVHLLITIYSALIFIPWYFLTNAKKKNAMAKRIKAKPTSDK

SEQ ID NO: 88 - protein, 145 aa, Homo sapiens
MKLKLNVLTIILLPVHLLITIYSALIFIPWYFLTNAKKKNAMAKRIKAKPTSDKPGSPYRSVTHFDSLAVIDIPGADTLDKLFDHAVSKFGKKDSLGTREILSEENEMQPNGKVFKKLILGNYKWMNYLEVNRRVNNFGSGLTAL

SEQ ID NO: 89 - protein, 126 aa, Homo sapiens
MKLKLNVLTIILLPVHLLITIYSALIFIPWYFLTNAKKKNAMAKRIKAKPTSDKPGSPYRSVTHFDSLAVIDIPGADTLDKLFDHAVSKFGKKDSLGTREILSEENEMQPNGKVFKKLILGNYKWM

SEQ ID NO: 90 - protein, 128 aa, Homo sapiens
MKLKLNVLTIILLPVHLLITIYSALIFIPWYFLTNAKKKNAMAKRIKAKPTSDKPGSPYRSVTHFDSLAVIDIPGADTLDKLFDHAVSKFGKKDSLGTREILSEENEMQPNGKVFKKLILGNYKWMNY

SEQ ID NO: 91 - protein, 80 aa, Homo sapiens
NLVRMRSQALGQSAPSLTASLKELSLPRRGSLIDSQKWNCLVKRSLSPTPSSPGSPCSPLLAFHFWSPVCPNAGRTSPLG

SEQ ID NO: 92 - protein, 58 aa, Homo sapiens
MAKVAKDLNPGVKKMSLGQLQSARGVACLGCKGTCSGFEPHSWRDCSTWSSSPRRSSQ

SEQ ID NO: 93 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 94 - protein, 760 aa, Homo sapiens; identical to SEQ ID NO: 93

SEQ ID NO: 95 - protein, 81 aa, Homo sapiens
MMDQARSAFSNLFGGEPLSYTRFSLARQVDGDNSHVEMKLAVDEEENADNNTKANVTKPKRCSGSICYGTIAVIVFFLIGC

SEQ ID NO: 96 - protein, 118 aa, Homo sapiens
AAAEVAGQFVIKLTHDVELNLDYERYNSQLLSFVRDLNQYRADIKEMGLSLQWLYSARGDFFRATSRLTTDFGNAEKTDRFVMKKLNDRVMREGPQMMLLLTDARPSNHFLSPLLSLH

SEQ ID NO: 97 - protein, 643 aa, Homo sapiens
MHHLLEQSADMATALLAGEKLRELILPGAQDDKAGALAALLLQLKLELPFDRVVTIGTVLVPILLVTLVFTKNFAEEPIYCYTPHNFTRDQALYARGYCWTELRDALPGVDASLWPSLFEHKFLPYALLAFAAIMYVPALGWEFLASTRLTSELNFLLQEIDNCYHRAAEGRAPKIEKQIQSKGPGITEREKREIIENAEKEKSPEQNLFEKYLERRGRSNFLAKLYLARHVLILLLSAVPISYLCTYYATQKQNEFTCALGASPDGAAGAGPAVRVSCKLPSVQLQRIIAGVDIVLLCVMNLIILVNLIHLFIFRKSNFIFDKLHKVGIKTRRQWRRSQFCDINILAMFCNENRDHIKSLNRLDFITNESDLMYDNVVRQLLAALAQSNHDATPTVRDSGVQTVDPSANPAEPDGAAEPPVVKRPRKKMKWIPTSNPLPQPFKEPLAIMRVENSKAEKPKPARRKTATDTLIAPLLDRSAHHYKGGGGDPGPGPAPAPAPPPAPDKKHARHFSLDVHPYILGTKKAKAEAVPAALPASRSQEGGFLSQAEDCGLGLAPAPIKDAPLPEKEIPYPTEPARAGLPSGGPFHVRSPPAAPAVAPLTPASLGKAEPLTILSRNATHPLLHINTLSSSPPSTSRERS

SEQ ID NO: 98 - protein, 677 aa, Homo sapiens
MHHLLEQSADMATALLAGEKLRELILPGAQDDKAGALAALLLQLKLELPFDRVVTIGTVLVPILLVTLVFTKNFAEEPIYCYTPHNFTRDQALYARGYCWTELRDALPGVDASLWPSLFEHKFLPYALLAFAAIMYVPALGWEFLASTRLTSELNFLLQEIDNCYHRAAEGRAPKIEKQIQSKGPGITEREKREIIENAEKEKSPEQNLFEKYLERRGRSNFLAKLYLARHVLILLLSAVPISYLCTYYATQKQNEFTCALGASPDGAAGAGPAVRVSCKLPSVQLQRIIAGVDIVLLCVMNLIILVNLIHLFIFRKSNFIFDKLHKVGIKTRRQWRRSQFCDINILAMFCNENRDHIKSLNRLDFITNESDLMYDNVVRQLLAALAQSNHDATPTVRDSGVQTVDPSANPAEPDGAAEPPVVKRPRKKMKWIPTSNPLPQPFKEPLAIMRVENSKAEKPKPARRKTATDTLIAPLLDRSAHHYKGGGGDPGPGPAPAPAPPPAPDKKHARHFSLDVHPYILGTKKAKAEAVPAALPASRSQEGGFLSQAEDCGLGLAPAPIKDAPLPEKEIPYPTEPARAGLPSGGPFHVRSPPAAPAVAPLTPASLGKAEPLTILSRNATHPLLHINTLYEAREEEDGGPRLPQDVGDLIAIPAPQQILIATFDEPRTVVSTVEF

SEQ ID NO: 99 - protein, 77 aa, Homo sapiens
MATALLAGEKLRELILPGAQDDKAGALAALLLQLKLELPFDRVVTIGTVLVPILLVTLVFTKNFAGWTLFSGSWDWL

SEQ ID NO: 100 - protein, 1204 aa, Homo sapiens
MYVTMMMTDQIPLELPPLLNGEVAMMPHLVNGDAAQQVILVQVNPGETFTIRAEDGTLQCIQGPAEVPMMSPNGSIPPIHVPPGYISQVIEDSTGVRRVVVTPQSPECYPPSYPSAMSPTHHLPPYLTHHPHFIHNSHTAYYPPVTGPGDMPPQFFPQHHLPHTIYGEQEIIPFYGMSTYITREDQYSKPPHKKLKDRQIDRQNRLNSPPSSIYKSSCTTVYNGYGKGHSGGSGGGGSGSGPGIKKTERRARSSPKSNDSDLQEYELEVKRVQDILSGIEKPQVSNIQARAVVLSWAPPVGLSCGPHSGLSFPYSYEVALSDKGRDGKYKIIYSGEELECNLKDLRPATDYHVRVYAMYNSVKGSCSEPVSFTTHSCAPECPFPPKLAHRSKSSLTLQWKAPIDNGSKITNYLLEWDEGKRNSGFRQCFFGSQKHCKLTKLCPAMGYTFRLAARNDIGTSGYSQEVVCYTLGNIPQMPSAPRLVRAGITWVTLQWSKPEGCSPEEVITYTLEIQEDENDNLFHPKYTGEDLTCTVKNLKRSTQYKFRLTASNTEGKSCPSEVLVCTTSPDRPGPPTRPLVKGPVTSHGFSVKWDPPKDNGGSEILKYLLEITDGNSEANQWEVAYSGSATEYTFTHLKPGTLYKLRACCISTGGHSQCSESLPVRTLSIAPGQCRPPRVLGRPKHKEVHLEWDVPASESGCEVSEYSVEMTEPEDVASEVYHGPELECTVGNLLPGTVYRFRVRALNDGGYGPYSDVSEITTAAGPPGQCKAPCISCTPDGCVLVGWESPDSSGADISEYRLEWGEDEESLELIYHGTDTRFEIRDLLPAAQYCCRLQAFNQAGAGPYSELVLCQTPASAPDPVSTLCVLEEEPLDAYPDSPSACLVLNWEEPCNNGSEILAYTIDLGDTSITVGNTTMHVMKDLLPETTYRIRIQAINEIGAGPFSQFIKAKTRPLPPLPPRLECAAAGPQSLKLKWGDSNSKTHAAEDIVYTLQLEDRNKRFISIYRGPSHTYKVQRLTEFTCYSFRIQAASEAGEGPFSETYTFSTTKSVPPTIKAPRVTQLEGNSCEILWETVPSMKGDPVNYILQVLVGRESEYKQVYKGEEATFQISGLQTNTDYRFRVCACRRCLDTSQELSGAFSPSAAFVLQRSEVMLTGDMGSLDDPKMKSMMPTDEQFAAIIVLGFATLSILFAFILQYFLMK

SEQ ID NO: 101 - protein, 1204 aa, Homo sapiens; identical to SEQ ID NO: 100

SEQ ID NO: 102 - protein, 1204 aa, Homo sapiens; identical to SEQ ID NO: 100

SEQ ID NO: 103 - protein, 71 aa, Homo sapiens
MYVTMMMTDQIPLELPPLLNGEVAMMPHLVNGDAAQQVILVQVNPGETFTIRAEDGTLQCIQDEVVKRACD

SEQ ID NO: 104 - protein, 71 aa, Homo sapiens; identical to SEQ ID NO: 103

SEQ ID NO: 105 - protein, 862 aa, Homo sapiens
MAHTFRGCSLAFMFIITWLLIKAKIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEEEPTGMLDVWYMKRHIDYSRQQISLFWKNLSVSEARGKILHYQVTLQELTGGKAMTQNITGHTSWTTVIPRTGNWAVAVSAANSKGSSLPTRINIMNLCEAGLLAPRQVSANSEGMDNILVTWQPPRKDPSAVQEYVVEWRELHPGGDTQVPLNWLRSRPYNVSALISENIKSYICYEIRVYALSGDQGGCSSILGNSKHKAPLSGPHINAITEEKGSILISWNSIPVQEQMGCLLHYRIYWKERDSNSQPQLCEIPYRVSQNSHPINSLQPRVTYVLWMTALTAAGESSHGNEREFCLQGKANWMAFVAPSICIAIIMVGIFSTHYFQQKVFVLLAALRPQWCSREIPDPANSTCAKKYPIAEEKTQLPLDRLLIDWPTPEDPEPLVISEVLHQVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 106 - protein, 659 aa, Homo sapiens
MAHTFRGCSLAFMFIITWLLIKAKIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEEEPTGMLDVWYMKRHIDYSRQQISLFWKNLSVSEARGKILHYQVTLQELTGGKAMTQNITGHTSWTTVIPRTGNWAVAVSAANSKGSSLPTRINIMNLCEAGLLAPRQVSANSEGMDNILVTWQPPRKDPSAVQEYVVEWRELHPGGDTQVPLNWLRSRPYNVSALISENIKSYICYEIRVYALSGDQGGCSSILGNSKHKAPLSGPHINAITEEKGSILISWNSIPVQEQMGCLLHYRIYWKERDSNSQPQLCEIPYRVSQNSHPINSLQPRVTYVLWMTALTAAGESSHGNEREFCLQGKANWMAFVAPSICIAIIMVGIFSTHYFQQKRRHSCPWTGS

SEQ ID NO: 107 - protein, 230 aa, Homo sapiens
XQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEEEPTGMLDVWYMKRHIDYSRQQISLFWKVSFKRQLKTQGNKTEG

SEQ ID NO: 108 - protein, 635 aa, Homo sapiens
MAHTFRGCSLAFMFIITWLLIKAKIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEEEPTGMLDVWYMKRHIDYSRQQISLFWKNLSVSEARGKILHYQVTLQELTGGKAMTQNITGHTSWTTVIPRTGNWAVAVSAANSKGSSLPTRINIMNLCEAGLLAPRQVSANSEGMDNILVTWQPPRKDPSAVQEYVVEWRELHPGGDTQVPLNWLRSRPYNVSALISENIKSYICYEIRVYALSGDQGGCSSILGNSKHKAPLSGPHINAITEEKGSILISWNSIPVQEQMGCLLHYRIYWKERDSNSQPQLCEIPYRVSQNSHPINSLQPRVTYVLWMTALTAAGESSHGNEREFCLQGEDTAALGQAPDRLAHA

SEQ ID NO: 109 - protein, 776 aa, Homo sapiens
MAHTFRGCSLAFMFIITWLLIKAKIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEEEPTGMLDVWYMKRHIDYSRQQISLFWKNLSVSEARGKILHYQVTLQELTGGKAMTQNITGHTSWTTVIPRTGNWAVAVSAANSKGSSLPTRINIMNLCEAGLLAPRQVSANSEGMDNILVTWQPPRKDPSAVQEYVVEWRELHPGGDTQVPLNWLRSRPYNVSALISEIPYRVSQNSHPINSLQPRVTYVLWMTALTAAGESSHGNEREFCLQGKANWMAFVAPSICIAIIMVGIFSTHYFQQKVFVLLAALRPQWCSREIPDPANSTCAKKYPIAEEKTQLPLDRLLIDWPTPEDPEPLVISEVLHQVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 110 - protein, 370 aa, Homo sapiens
MLPRRLLAAWLAGTRGGGLLALLANQCRFVTGLRVRRAQQIAQLYGRLYSESSRRVLLGRLWRRLHGRPGHASALMAALAGVFVWDEERIQEEELQRSINEMKRLEEMSNMFQSSGVQHHPPEPKAQTEGNEDSEGKEQRWEMVMDKKHFKLWRRPITGTHLYQYRVFGTYTDVTPRQFFNVQLDTEYRKKWDALVIKLEVIERDVVSGSEVLHWVTHFPYPMYSRDYVYVRRYSVDQENNMMVLVSRAVEHPSVPESPEFVRVRSYESQMVIRPHKSFDENGFDYLLTYSDNPQTVFPRYCVSWMVSSGMPDFLEKLHMATLKAKNMEIKVKDYISAKPLEMSSEAKATSQSSERKNEGSCGPARIEYA

SEQ ID NO: 111 - protein, 154 aa, Homo sapiens
MVSKAKVIVNGWLFPACLGFCLSESFFMVKGAALFLQQGSSPQGQRSLQHPHKHAGDLPQHLQVMINLLRCEDRIKLAVRLESAWADRVRYMVVVYSSGRQDTEENILLGVDFSSKESKSCTIGMVLRLWSDTKIHLDGDGGFSVSTAGRMHIF

SEQ ID NO: 112 - protein, 37 aa, Homo sapiens
MALVTLQRSPTPSAASSSASNSELEAGSEEDRKLNLR

SEQ ID NO: 113 - protein, 147 aa, Homo sapiens
MAGNSILLAAVSILSACQQSYFALQVGKARLKYKVTPPAVTGSPEFERVFRAQQNCVEFYPIFIITLWMAGWYFNQVFATCLGLVYIYGRHLYFWGYSEAAKKRITGFRLSLGILALLTLLGALGIANSFLDEYLDLNIAKKLRRQF

SEQ ID NO: 114 - protein, 147 aa, Homo sapiens; identical to SEQ ID NO: 113

SEQ ID NO: 115 - protein, 77 aa, Homo sapiens
MAGNSILLAAVSILSACQQSYFALQVGKARLKYKVTPPAVTGSPEFERVFRAHFCYLSGSGVHIWPSPILLGIFRSC

SEQ ID NO: 116 - protein, 147 aa, Homo sapiens; identical to SEQ ID NO: 113

SEQ ID NO: 117 - protein, 110 aa, Homo sapiens
MFKSPLKVPALIQCLGMYDSSLAAKYLLHSLVFGSAVYSSGSERHLTYIQKIFRMEIFGCFALTELSHGSNTKAIRTTAHYDPATEEFIIHSPDFEAAKFWVGNMGKTAT

SEQ ID NO: 118 - protein, 121 aa, Homo sapiens
XGHQNVFPNHISVGTNRKRCLEDSEDFGVKKARTEAQSLDSAVPLTNGDTEDDADKMHVDREFAVVTGGSGQFPVSCNNNPMVEDTKQQESGSVGPKEIEIYTVSAMQTPCRCRNQYAYYF

SEQ ID NO: 119 - protein, 37 aa, Homo sapiens
MGVQVETISPGDGRTFPKRGQTCVVHYTGGDCILVNE

SEQ ID NO: 120 - protein, 129 aa, Homo sapiens
MGVQVETISPGDGRTFPKRGQTCVVHYTGMLEDGKKFDSSRDRNKPFKFMLGKQEVIRGWEEGVAQVCSLICFVLLSSIYLLYFFIGGIFIRFKNKKSEKSHPHAIPSDQYPHKKLLLLMFLVLWFKHI

SEQ ID NO: 121 - protein, 97 aa, Homo sapiens
MGVQVETISPGDGRTFPKRGQTCVVHYTDECGSESQTDYISRLCLWCHWAPRHHPTTCHSRLRCGASKTGMTGMASSLSSLFLDLPWRDLVPPDMCT

SEQ ID NO: 122 - protein, 276 aa, Homo sapiens
MPVPASWPHPPGPFLLLTLLLGLTEVAGEEELQMIQPEKLLLVTVGKTATLHCTVTSLLPVGPVLWFRGVGPGRELIYNQKEGHFPRVTTVSDLTKRNNMDFSIRISSITPADVGTYYCVKFRKGSPENVEFKSGPGTEMALGAKPSAPVVLGPAARTTPEHTVSFTCESHGFSPRDITLKWFKNGNELSDFQTNVDPTGQSVAYSIRSTARVVLDPWDVRSQVICEVAHVTLQGDPLRGTANLSEAIRGPASSLTALLLIAVLLGPIYVPWKQKT

SEQ ID NO: 123 - protein, 387 aa, Homo sapiens
MPVPASWPHPPGPFLLLTLLLGLTEVAGEEELQMIQPEKLLLVTVGKTATLHCTVTSLLPVGPVLWFRGVGPGRELIYNQKEGHFPRVTTVSDLTKRNNMDFSIRISSITPADVGTYYCVKFRKGSPENVEFKSGPGTEMALGAKPSAPVVLGPAARTTPEHTVSFTCESHGFSPRDITLKWFKNGNELSDFQTNVDPTGQSVAYSIRSTARVVLDPWDVRSQVICEVAHVTLQGDPLRGTANLSEAIRVPPTLEVTQQPMRVGNQVNVTCQVRKFYPQSLQLTWSENGNVCQRETASTLTENKDGTYNWTSWFLVNISDQRDDVVLTCQVKHDGQLAVSKRLALEVTVHQKDQSSDATPGPASSLTALLLIAVLLGPIYVPWKQKT

SEQ ID NO: 124 - protein, 170 aa, Homo sapiens
MPVPASWPHPPGPFLLLTLLLGLTEVAGEEELQMIQPEKLLLVTVGKTATLHCTVTSLLPVGPVLWFRGVGPGRELIYNQKEGHFPRVTTVSDLTKRNNMDFSIRISSITPADVGTYYCVKFRKGSPENVEFKSGPGTEMALGGPASSLTALLLIAVLLGPIYVPWKQKT

SEQ ID NO: 125 - protein, 354 aa, Homo sapiens
MIQPEKLLLVTVGKTATLHCTVTSLLPVGPVLWFRGVGPGRELIYNQKEGHFPRVTTVSDLTKRNNMDFSIRISSITPADVGTYYCVKFRKGSPENVEFKSGPGTEMALGAKPSAPVVLGPAARTTPEHTVSFTCESHGFSPRDITLKWFKNGNELSDFQTNVDPTGQSVAYSIRSTARVVLDPWDVRSQVICEVAHVTLQGDPLRGTANLSEAIRVPPTLEVTQQPMRVGNQVNVTCQVRKFYPQSLQLTWSENGNVCQRETASTLTENKDGTYNWTSWFLVNISDQRDDVVLTCQVKHDGQLAVSKRLALEVTVHQKDQSSDATPGPASSLTALLLIAVLLGPIYVPWKQKT

SEQ ID NO: 126 - protein, 276 aa, Homo sapiens; identical to SEQ ID NO: 122

SEQ ID NO: 127 - protein, 556 aa, Homo sapiens
MHGSLEALLFLPQVTLSLAHAHLICSNAQLEMCVFPHRFLGDLTTEGINKPGFYKGPAGSQVTLSSLGNQTRVLLEEQARHLLNEQEHATMAYYLDEYRGGSVSVEALVMALFKLLNTHAKFSLLSEVRGTISPQDLERFDHLVLRREIESMKARQPPGPGAGDTYSMVSYSDTGSSTGSHGTSTTVSSARNTLDLEETGEAVQGNINALPDVSVDDVRSTSQGLSSFKPLPRPPPLAQGNDLPLGQPRKLGREDLQPPSSMPSCSGTVFSAPQNRSPPAGTAPTPGTSSAQDLPSSPIYASVSPANPSSKRPLDAHLALVNQHPIGPFPRVQSPPHLKSPSAEATVAGGCLLPPSPSGHPDQTGTNQHFVMVEVHRPDSEPDVNEVRALPQTRTASTLSQLSDSGQTLSEDSGVDAGEAEASAPGRGRQSVSTKSRSSKELPRNERPTDGANKPPGLLEPTSTLVRVKKSAATLGIAIEGGANTRQPLPRIVTIQRGGSAHNCGQLKVGHVILEVNGLTLRGKEHREAARIIAEAFKTKDRDYIDFLVTEFNVML

SEQ ID NO: 128 - protein, 180 aa, Homo sapiens
MSVLRPLDKLPGLNTATILLVGTEDALLQQLADSMLKEDCASELKVHLAKSLPLPSSVNRPRIDLIVFVVNLHSKYSLQNTEESLRHVDASFFLGKVCFLATGAGRESHCSIHRHTVVKLAHTYQSPLLYCDLEVEGFRATMAQRLVRVLQICAGHVPGVSALNLLSLLRSSEGPSLEDL

SEQ ID NO: 129 - protein, 146 aa, Homo sapiens
MLKEDCASELKVHLAKSLPLPSSVNRPRIDLIVFVVNLHSKYSLQNTEESLRHVDASFFLGKVCFLATGAGRESHCSIHRHTVVKLAHTYQSPLLYCDLEVEGFRATMAQRLVRVLQICAGHVPGVSALNLLSLLRSSEGPSLEDL

SEQ ID NO: 130 - protein, 125 aa, Homo sapiens
MLKEDCASELKVHLAKSLPLPSSVNRPRIDLIVFVVNLHSKYRIREARTSAFSVVKFCSLVCFLTLAWPPQSPEHRGVPAPCGCQLLLGEGVFPRHRCWAGEPLQHSPAHRGEAGPHLSKPPALL

SEQ ID NO: 131 - protein, 73 aa, Homo sapiens
MLKEDCASELKVHLAKSLPLPSSVNRPRIDLIVFVVNLHSKYSLQNTEESLRHVDASFFLGKVCFLATGGGRL

SEQ ID NO: 132 - protein, 107 aa, Homo sapiens
MSVLRPLDKLPGLNTATILLVGTEDALLQQLADSMLKEDCASELKVHLAKSLPLPSSVNRPRIDLIVFVVNLHSKYSLQNTEESLRHVDASFFLGKVCFLATGGGRL

SEQ ID NO: 133 - protein, 166 aa, Homo sapiens
MRIPALNAYMKSLLSLPVWVLMDEDVRIFFYQSPYDSEQVPQALRRLRPRTRKVKSVSPQGNSVDRMAAPRAEALFDFTGNSKLELNFKAGDVIFLLSRINKDWLEGTVRGATGIFPLSFVKILKDFPEEDDPTNWLRCYYYEDTISTIKSVAWEGGACPAFLPSL

SEQ ID NO: 134 - protein, 889 aa, Homo sapiens
MVLAQGLLSMALLALCWERSLAGAEETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGYNGIWSEWSEARSWDTESVLPMWVLALIVIFLTIAVLLALRFCGIYGYRLRRKWEEKIPNPSKSHLFQNGSAELWPPGSMSAFTSGSPPHQGPWGSRFPELEGVFPVGFGDSEVSPLTIEDPKHVCDPPSGPDTTPAASDLPTEQPPSPQPGPPAASHTPEKQASSFDFNGPYLGPPHSRSLPDILGQPEPPQEGGSQKSPPPGSLEYLCLPAGGQVQLVPLAQAMGPGQAVEVERRPSQGAAGSPSLESGGGPAPPALGPRVGGQDQKDSPVAIPMSSGDTEDPGVASGYVSSADLVFTPNSGASSVSLVPSLGLPSDQTPSLCPGLASGPPGAPGPVKSGFEGYVELPPIEGRSPRSPRNNPVPPEAKSPVLNPGERPADVSPTSPQPEGLLVLQQVGDYCFLPGLGPGPLSLRSKPSSPGPGPEIKNLDQAFQVKKPPGQAVPQVPVIQLFKALVVSVMSVGPPVP

SEQ ID NO: 135 - protein, 897 aa, Homo sapiens
MVLAQGLLSMALLALCWERSLAGAEETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGYNGIWSEWSEARSWDTESVLPMWVLALIVIFLTIAVLLALRFCGIYGYRLRRKWEEKIPNPSKSHLFQNGSAELWPPGSMSAFTSGSPPHQGPWGSRFPELEGVFPVGFGDSEVSPLTIEDPKHVCDPPSGPDTTPAASDLPTEQPPSPQPGPPAASHTPEKQASSFDFNGPYLGPPHSRSLPDILGQPEPPQEGGSQKSPPPGSLEYLCLPAGGQVQLVPLAQAMGPGQAVEVERRPSQGAAGSPSLESGGGPAPPALGPRVGGQDQKDSPVAIPMSSGDTEDPGVASGYVSSADLVFTPNSGASSVSLVPSLGLPSDQTPSLCPGLASGPPGAPGPVKSGFEGYVELPPIEGRSPRSPRNNPVPPEAKSPVLNPGERPADVSPTSPQPEGLLVLQQVGDYCFLPGLGPGPLSLRSKPSSPGPGPEIKNLDQAFQVKKPPGQAVPQVPVIQLFKALKQQDYLSLPPWEVNKPGEVC

SEQ ID NO: 136 - protein, 903 aa, Homo sapiens
MVLAQGLLSMALLALCWERSLAGAEETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGSAVLLREEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGYNGIWSEWSEARSWDTESVLPMWVLALIVIFLTIAVLLALRFCGIYGYRLRRKWEEKIPNPSKSHLFQNGSAELWPPGSMSAFTSGSPPHQGPWGSRFPELEGVFPVGFGDSEVSPLTIEDPKHVCDPPSGPDTTPAASDLPTEQPPSPQPGPPAASHTPEKQASSFDFNGPYLGPPHSRSLPDILGQPEPPQEGGSQKSPPPGSLEYLCLPAGGQVQLVPLAQAMGPGQAVEVERRPSQGAAGSPSLESGGGPAPPALGPRVGGQDQKDSPVAIPMSSGDTEDPGVASGYVSSADLVFTPNSGASSVSLVPSLGLPSDQTPSLCPGLASGPPGAPGPVKSGFEGYVELPPIEGRSPRSPRNNPVPPEAKSPVLNPGERPADVSPTSPQPEGLLVLQQVGDYCFLPGLGPGPLSLRSKPSSPGPGPEIKNLDQAFQVKKPPGQAVPQVPVIQLFKALKQQDYLSLPPWEVNKPGEVC

SEQ ID NO: 137 - protein, 229 aa, Homo sapiens
MPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIP

SEQ ID NO: 138 - protein, 144 aa, Homo sapiens
MAFTFAAFCYMLALLLTAALIFFAIWHIIAFDELKTDYKNPIDQCNTLNPLVLPEYLIHAFFCVMFLCAAEWLTLGLNMPLLAYHIWRYMSRPVMSGPGLYDPTTIMNADILAYCQKEGWCKLAFYLLAFFYYLYGMIYVLVSS

SEQ ID NO: 139 - protein, 96 aa, Homo sapiens
MAFTFAAFCYMLALLLTAALIFFAIWHIIAFDELKTDYKNPIDQCNTLNPLVLPEYLIHAFFCVMFLCAAEWLTLGLNMPLLAYHIWSMIYVLVSS

SEQ ID NO: 140 - protein, 121 aa, Homo sapiens
MAFTFAAFCYMLALLLTAALIFFAIWHLVLPEYLIHAFFCVMFLCAAEWLTLGLNMPLLAYHIWRYMSRPVMSGPGLYDPTTIMNADILAYCQKEGWCKLAFYLLAFFYYLYGMIYVLVSS

SEQ ID NO: 141 - protein, 33 aa, Homo sapiens
MAFTFAAFCYMLALLLTAALIFFAIWHDKEEFG

SEQ ID NO: 142 - protein, 136 aa, Homo sapiens
MAFTFAAFCYMLALLLTAALIFFAIWHIIAFDELKTDYKNPIDQCNTLNPLVLPEYLIHAFFCVMFLCAAEWLTLGLNMPLLAYHIWRYMSRPVMSGPGLYDPTTIMNADILAYCQKEGWCKLAFYLLAFFYYLYG

SEQ ID NO: 143 - protein, 53 aa, Homo sapiens
MAFTFAAFCYMLALLLTAALIFFAIWHIIAFDELKTDYKNPIDQCNTLNPVYE

SEQ ID NO: 144 - protein, 160 aa, Homo sapiens
MAFTFAAFCYMLALLLTAALIFFAIWHIIAFDELKTDYKNPIDQCNTLNPTVEKVKKIKRVKIALKLVLPEYLIHAFFCVMFLCAAEWLTLGLNMPLLAYHIWRYMSRPVMSGPGLYDPTTIMNADILAYCQKEGWCKLAFYLLAFFYYLYGMIYVLVSS

SEQ ID NO: 145 - protein, 435 aa, Homo sapiens
MAAPLVLVLVVAVTVRAALFRSSLAEFISERVEVVSPLSSWKRVVEGLSLLDLGVSPYSGAVFHETPLIIYLFHFLIDYAELVFMITDALTAIALYFAIQDFNKVVFKKQKLLLELDQYAPDVAELIRTPMEMRYIPLKVALFYLLNPYTILSCVAKSTCAINNTLIAFFILTTIKGSAFLSAIFLALATYQSLYPLTLFVPGLLYLLQRQYIPVKMKSKAFWIFSWEYAMMYVGSLVVIICLSFFLLSSWDFIPAVYGFILSVPDLTPNIGLFWYFFAEMFEHFSLFFVCVFQINVFFYTIPLAIKLKEHPIFFMFIQIAVIAIFKSYPTVGDVALYMAFFPVWNHLYRFLRNIFVLTCIIIVCSLLFPVLWHLWIYAGSANSNFFYAITLTFNVGQILLISDYFYAFLRREYYLTHGLYLTAKDGTEAMLVLK

SEQ ID NO: 146 - protein, 415 aa, Homo sapiens
MAAPLVLVLVVAVTVRAALFRSSLAEFISERVEVVSPLSSWKRVVEGLSLLDLGVSPYSGAVFHEITDALTAIALYFAIQDFNKVVFKKQKLLLELDQYAPDVAELIRTPMEMRYIPLKVALFYLLNPYTILSCVAKSTCAINNTLIAFFILTTIKGSAFLSAIFLALATYQSLYPLTLFVPGLLYLLQRQYIPVKMKSKAFWIFSWEYAMMYVGSLVVIICLSFFLLSSWDFIPAVYGFILSVPDLTPNIGLFWYFFAEMFEHFSLFFVCVFQINVFFYTIPLAIKLKEHPIFFMFIQIAVIAIFKSYPTVGDVALYMAFFPVWNHLYRFLRNIFVLTCIIIVCSLLFPVLWHLWIYAGSANSNFFYAITLTFNVGQILLISDYFYAFLRREYYLTHGLYLTAKDGTEAMLVLK

SEQ ID NO: 147 - protein, 170 aa, Homo sapiens [sequence omitted]

SEQ ID NO: 148 - protein, 336 aa, Homo sapiens
MARRRSQRVCASGPSMLNSARGAPELLRGTATNAEVSAAAAGATGSEELPPGDRGCRNGGGRGPAATTSSTGVAVGAEHGEDSLSRKPDPEPGRMDHHQPGTGRYQVLLNEEDNSESSAIEQPPTSNPAPQIVQAASSAPALETDSSPPPYSSITVEVPTTSDTEVYGEFYPVPPPYSVATSLPTYDEAEKAKAAAMAAAAAETSQRIQEEECPPRDDFSDADQLRVGNDGIFMLAFFMAFIFNWLGFCLSFCITNTIAGRYGAICGFGLSLIKWILIVRFSDYFTGYFNGQYWLWWIFLVLGLLLFFRGFVNYLKVRNMSESMAAAHRTRYFFLL

SEQ ID NO: 149 - protein, 222 aa, Homo sapiens
MDHHQPGTGRYQVLLNEEDNSESSAIEQPPTSNPAPQIVQAASSAPALETDSSPPPYSSITVEVPTTSDTEVYGEFYPVPPPYSVATSLPTYDEAEKAKAAAMAAAAAETSQRIQEEECPPRDDFSDADQLRVGNDGIFMLAFFTGRYGAICGFGLSLIKWILIVRFSDYFTGYFNGQYWLWWIFLVLGLLLFFRGFVNYLKVRNMSESMAAAHRTRYFFLL

SEQ ID NO: 150 - protein, 336 aa, Homo sapiens; identical to SEQ ID NO: 148

SEQ ID NO: 151 - protein, 242 aa, Homo sapiens
MDHHQPGTGRYQVLLNEEDNSESSAIEQPPTSNPAPQIVQAASSAPALETDSSPPPYSSITVEVPTTSDTEVYGEFYPVPPPYSVATSLPTYDEAEKAKAAAMAAAAAETSQRIQEEECPPRDDFSDADQLRVGNDGIFMLAFFMAFIFNWLGFCLSFCITNTIAGRYGAICGFGLSLIKWILIVRFSDYFTGYFNGQYWLWWIFLVLGLLLFFRGFVNYLKVRNMSESMAAAHRTRYFFLL

SEQ ID NO: 152 - protein, 325 aa, Homo sapiens
MDMWTALLILQALLLPSLADGATPALRFVAVGDWGGVPNAPFHTAREMANAKEIARTVQILGADFILSLGDNFYFTGVQDINDKRFQETFEDVFSDRSLRKVPWYVLAGNHDHLGNVSAQIAYSKISKRWNFPSPFYRLHFKIPQTNVSVAIFMLDTVTLCGNSDDFLSQQPERPRDVKLARTQLSWLKKQLAAAREDYVLVAGHYPVWSIAEHGPTHCLVKQLRPLLATYGVTAYLCGHDHNLQYLQDENGVGYVLSGAGNFMDPSKRHQRKVPNGYLRFHYGTEDSLGGFAYVEISSKEMTVTYIEASGKSLFKTRLPRRARP

SEQ ID NO: 153 - protein, 325 aa, Homo sapiens; identical to SEQ ID NO: 152

SEQ ID NO: 154 - protein, 325 aa, Homo sapiens; identical to SEQ ID NO: 152

SEQ ID NO: 155 - protein, 74 aa, Homo sapiens
MDMWTALLILQALLLPSLADGATPALRFVAVGDWGGVPNAPFHTAREMANAKEIARTVQILGADFILSLGDNFY

SEQ ID NO: 156 - protein, 59 aa, Homo sapiens
MDMWTALLILQALLLPSLGISWTPQSGTSARSPTAICASTMGLKTHWVALPMWRSAPKR

SEQ ID NO: 157 - protein, 124 aa, Homo sapiens
MDMWTALLILQALLLPSLADGATPALRFVAVGDWGGVPNAPFHTAREMANAKEIARTVQILGADFILSLGDNFYFTGVQDINDKRFQETFEDVFSDRSLRKVPWYVLAGNHDHLGNVSAQIAYS

SEQ ID NO: 158 - protein, 52 aa, Homo sapiens
MDMWTALLILQALLLPSLADGATPALRFVAVGDWGGVPNAPFHTAREMANAK

SEQ ID NO: 159 - protein, 325 aa, Homo sapiens; identical to SEQ ID NO: 152

SEQ ID NO: 160 - protein, 119 aa, Homo sapiens
MPSDFISLLSADLDLESPKSLYSRDSLKLHPSQNFHRAGLLEESVYDLLPKELQLPPSRETSVASMSQTSGGEAGSPPPAVVAAGIPEMFILCILMCMILRFKKGFASEAGSVCIKNDL

SEQ ID NO: 161 - protein, 150 aa, Homo sapiens
MSGSMATAEASGSDGKGQEVETSVTYYRLEEVAKRNSLKELWLVIHGRVYDVTRFLNEHPGGEEVLLEQAGVDASESFEDVGHSSDAREMLKQYYIGDIHPSDLKPESGSKDPSKNDTCKSCWAYWILPIIGAVLLGFLYRYYTSESKSS

SEQ ID NO: 162 - protein, 146 aa, Homo sapiens
MATAEASGSDGKGQEVETSVTYYRLEEVAKRNSLKELWLVIHGRVYDVTRFLNEHPGGEEVLLEQAGVDASESFEDVGHSSDAREMLKQYYIGDIHPSDLKPESGSKDPSKNDTCKSCWAYWILPIIGAVLLGFLYRYYTSESKSS

SEQ ID NO: 163 - protein, 64 aa, Homo sapiens
KRNSLKELWLVIHGRVYDVTRFLNEDPSKNDTCKSCWAYWILPIIGAVLLGFLYRYYTSESKSS

SEQ ID NO: 164 - protein, 317 aa, Homo sapiens
MTSRTRVTWPSPPRPLPVPAAAAVAFGAKGTDPAEARSSRGIEEAGPRAHGRAGREPERRRSRQQRRGGLQARRSTLLKTCARARATAPGAMKMVAPWTRFYSNSCCLCCHVRTGTILLGVWYLIINAVVLLILLSALADPDQYNFSSSELGGDFEFMDDANMCIAIAISLLMILICAMATYGAYKQRAAWIIPFFCYQIFDFALNMLVAITVLIYPNSIQEYIRQLPPNFPYRDDVMSVNPTCLVLIILLFISIILTFKGYLISCVWNCYRYINGRNSSDVLVYVTSNDTTVLLPPYDDATVNGAAKEPPPPYVSA

SEQ ID NO: 165 - protein, 152 aa, Homo sapiens
MKMVAPWTRFYSNSCCLCCHVRTGTILLGVWYLIINAVVLLILLSALADPDQYNFSSSELGGDFEFMDDAKILFNLSADMCIAIAISLLMILICAMATYGAYKQRAAWIIPFFCYQIFDFALNMLVAITVLIYPNSIQEYIRQLPPNFPYRD

SEQ ID NO: 166 - protein, 226 aa, Homo sapiens
MKMVAPWTRFYSNSCCLCCHVRTGTILLGVWYLIINAVVLLILLSALADPDQYNFSSSELGGDFEFMDDANMCIAIAISLLMILICAMATYGAYKQRAAWIIPFFCYQIFDFALNMLVAITVLIYPNSIQEYIRQLPPNFPYRDDVMSVNPTCLVLIILLFISIILTFKGYLISCVWNCYRYINGRNSSDVLVYVTSNDTTVLLPPYDDATVNGAAKEPPPPYVSA

SEQ ID NO: 167 - protein, 317 aa, Homo sapiens; identical to SEQ ID NO: 164

SEQ ID NO: 168 - protein, 177 aa, Homo sapiens
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 169 - protein, 115 aa, Homo sapiens
MFHVSFRYIFGLPPLILVLLPVASSDCDIEGKDGKQYESVLMVSIDQLLDSMKEIGSNCLNNEFNFFKRHICDANKEENKSLKEQKKLNDLCFLKRLLQEIKTCWNKILMGTKEH

SEQ ID NO: 170 - protein, 71 aa, Homo sapiens
MFHVSFRYIFGLPPLILVLLPVASSDCDIEGKDGKQYESVLMVSIDQLLNQPYKKKEKRTERKQQSAMSNN

SEQ ID NO: 171 - protein, 37 aa, Homo sapiens
MFHVSFRYIFGLPPLILVLLPVASSDCDIEGQHERNW

SEQ ID NO: 172 - protein, 133 aa, Homo sapiens
MFHVSFRYIFGLPPLILVLLPVASSDCDIEGKDGKQYESVLMVSIDQLLDSMKEIGSNCLNNEFNFFKRHICDANKVKGRKPAALGEAQPTKSLEENKSLKEQKKLNDLCFLKRLLQEIKTCWNKILMGTKEH

SEQ ID NO: 173 - protein, 37 aa, Homo sapiens; identical to SEQ ID NO: 171

SEQ ID NO: 174 - protein, 159 aa, Homo sapiens
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQEENKSLKEQKKLNDLCFLKRLLQEIKTCWNKILMGTKEH

SEQ ID NO: 175 - protein, 229 aa, Homo sapiens
MTPQLLLALVLWASCPPCSGRKGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGK

SEQ ID NO: 176 - protein, 130 aa, Homo sapiens
XMLENWTRTSLEKQEQPHEDPERKGSLSNLMDFVKKTGICASKWEWGTTHNFLYKHGGIRDKIMSSRKHLHLVDAGLAINTPFPLVLPPTREVHLILSFDFSAGDPFEHPRSATSNELLCMSVSSAVKLG

SEQ ID NO: 177 - protein, 36 aa, Homo sapiens
MRTRPRPRLRLECNGMISAHWHLHLLSSSDSPPSAS

SEQ ID NO: 178 - protein, 47 aa, Homo sapiens
MKEQGLLDAVTYLAGVSGSTWAISSLYTNDGDMEALEADLKHRFTRQ

SEQ ID NO: 179 - protein, 43 aa, Homo sapiens
MRTRPRPRLRVSLLLPRLECNGMISAHWHLHLLSSSDSPPSAS

SEQ ID NO: 180 - protein, 194 aa, Homo sapiens [sequence omitted]

SEQ ID NO: 181 - protein, 312 aa, Homo sapiens
MLRAPGCLLRTSVAPAAALAAALLSSLARCSLLEPRDPVASSLSPYFGTKTRYEDVNPVLLSGPEAPWRDPELLEGTCTPVQLVALIRHGTRYPTVKQIRKLRQLHGLLQARGSRDGGASSTGSRDLGAALADWPLWYADWMDGQLVEKGRQDMRQLALRLASLFPALFSRENYGRLRLITSSKHRCMDSSAAFLQGLWQHYHPGLPPPDVADMEFGPPTVNDKLMRFFDHCEKFLTEVEKNATALYHVEAFKTGPEMQNILKKVAATLQVPVNDLNAGLSQFLLQSSSSLVMQRLFFHCFLSWATSKTRNP

SEQ ID NO: 182 - protein, 487 aa, Homo sapiens
MLRAPGCLLRTSVAPAAALAAALLSSLARCSLLEPRDPVASSLSPYFGTKTRYEDVNPVLLSGPEAPWRDPELLEGTCTPVQLVALIRHGTRYPTVKQIRKLRQLHGLLQARGSRDGGASSTGSRDLGAALADWPLWYADWMDGQLVEKGRQDMRQLALRLASLFPALFSRENYGRLRLITSSKHRCMDSSAAFLQGLWQHYHPGLPPPDVADMEFGPPTVNDKLMRFFDHCEKFLTEVEKNATALYHVEAFKTGPEMQNILKKVAATLQVPVNDLNADLIQVAFFTCSFDLAIKGVKSPWCDVFDIDDAKVLEYLNDLKQYWKRGYGYTINSRSSCTLFQDIFQHLDKAVEQKQRSQPISSPVILQFGHAETLLPLLSLMGYFKDKEPLTAYNYKKQMHRKFRSGLIVPYASNLIFVLYHCENAKTPKEQFRVQMLLNEKVLPLAYSQETVSFYEDLKNHYKDILQSCQTSEECELARANSTSDEL

SEQ ID NO: 183 - protein, 286 aa, Homo sapiens
MCLFQLCGLVRYMEFGPPTVNDKLMRFFDHCEKFLTEVEKNATALYHVEAFKTGPEMQNILKKVAATLQVPVNDLNADLIQVAFFTCSFDLAIKGVKSPWCDVFDIDDAKVLEYLNDLKQYWKRGYGYTINSRSSCTLFQDIFQHLDKAVEQKQRSQPISSPVILQFGHAETLLPLLSLMGYFKDKEPLTAYNYKKQMHRKFRSGLIVPYASNLIFVLYHCENAKTPKEQFRVQMLLNEKVLPLAYSQETVSFYEDLKNHYKDILQSCQTSEECELARANSTSDEL

SEQ ID NO: 184 - protein, 207 aa, Homo sapiens
MASFPPRVNEKEIVFLFFLLVRLRTIGELLAPAAPFDKKCGRENWTVAFAPDGSYFAWSQGHRTVKLVPWSQCLQNFLLHGTKNVTNSSSLRLPRQNSDGGQKNKPREHIIDCGDIVWSLAFGSSVPEKQSRCVNIEWHRFRFGQDQLLLATGLNNGRIKIWDVYTGKLLLNLVDHTEVVRDLTFAPDGSLILVSASRDKTLRVWDL

SEQ ID NO: 185 - protein, 74 aa, Homo sapiens
MASFPPRVNEKEIVRLRTIGELLAPAAPFDKKCGRENWTVAFAPDGSYFAWSQGHRTVKLVPWSQCLQNLKTPP

SEQ ID NO: 186 - protein, 54 aa, Homo sapiens
MASFPPRVNEKEIGNMMKVLRGHQNWVYSCAFSPDSSMLCSVGASKAVVAAILV

SEQ ID NO: 187 - protein, 78 aa, Homo sapiens
MASFPPRVNEKEIAPAAPFDKKCGRENWTVAFAPDGSYFAWSQGHRTVKLVPWSQCLQNFLLHGTKNVTNSSSLRLPR

SEQ ID NO: 188 - protein, 276 aa, Homo sapiens
MAGPAALSAAAAAALAAALLLLRREDPGPGAGPSMAETEALSKLREDFRMQNKSVFILGASGETGRVLLKEILEQGLFSKVTLIGRRKLTFDEEAYKNVNQEVVDFEKLDDYASAFQGHDVGFCCLGTTRGKAGAEGFVRVDRDYVLKSAELAKAGGCKHFNLLSSKGADKSSNFLYLQVKGEVEAKVEELKFDRYSVFRPGVLLCDRQESRPGEWLVRKFFGSLPDSWASGHSVPVVTVVRAMLNNVVRPRDKQMELLENKAIHDLGKAHGSLKP

SEQ ID NO: 189 - protein, 133 aa, Homo sapiens
MAETEALSKLREDFRMQNKSVFILGASGETGRVLLKEILEQGLFSKVTLIGRRKLTFDEEAYKNVNQEVVDFEKLDDYASAFQGHDVGFCCLGTTRGKAGAVRKAYALFPFCWPVISRILFLLTLFLCACCNA

SEQ ID NO: 190 - protein, 133 aa, Homo sapiens; identical to SEQ ID NO: 189

SEQ ID NO: 191 - protein, 133 aa, Homo sapiens; identical to SEQ ID NO: 189

SEQ ID NO: 192 - protein, 463 aa, Homo sapiens
MGAGPSLLLAALLLLLSGDGAVRCDTPANCTYLDLLGTWVFQVGSSGSQRDVNCSVMGPQEKKVVVYLQKLDTAYDDLGNSGHFTIIYNQGFEIVLNDYKWFAFFKYKEEGSKVTTYCNETMTGWVHDVLGRNWACFTGKKVGTASENVYVNIAHLKNSQEKYSNRLYKYDHNFVKAINAIQKSWTATTYMEYETLTLGDMIRRSGGHSRKIPRPKPAPLTAEIQQKILHLPTSWDWRNVHGINFVSPVRNQASCGSCYSFASMGMLEARIRILTNNSQTPILSPQEVVSCSQYAQGCEGGFPYLIAGKYAQDFGLVEEACFPYTGTDSPCKMKEDCFRYYSSEYHYVGGFYGGCNEALMKLELVHHGPMAVAFEVYDDFLHYKKGIYHHTGLRDPFNPFELTNHAVLLVGYGTDSASGMDYWIVKNSWGTGWGENGYFRIRRGTDECAIESIAVAATPIPKL

SEQ ID NO: 193 - protein, 137 aa, Homo sapiens
MGAGPSLLLAALLLLLSGDGAVRCDTPANCTYLDLLGTWVFQVGSSGSQRDVNCSVMGPQEKKVVVYLQKLDTAYDDLGNSGHFTIIYNQGFEIVLNDYKWFAFFKDVTDFISHLFMQLGTVGIYDLPHLRNKLVIK

SEQ ID NO: 194 - protein, 245 aa, Homo sapiens
XVVDSPLARSARLPGPQEKKVVVYLQKLDTAYDDLGNSGHFTIIYNQGFEIVLNDYKWFAFFKYKEEGSKVTTYCNETMTGWVHDVLGRNWACFTGKKVGTASENVYVNIAHLKNSQEKYSNRLYKYDHNFVKAINAIQKSWTATTYMEYETLTLGDMIRRSGGHSRKIPRPKPAPLTAEIQQKILHLPTSWDWRNVHGINFVSPVRNQASCGSCYSFASMGMLEARIRILTNNSQTPILSPQEV

SEQ ID NO: 195 - protein, 36 aa, Homo sapiens
XDVTDFISHLFMQLGTVGIYDLPHLRNKLAMNRRWG

SEQ ID NO: 196 - protein, 141 aa, Homo sapiens
MGAGPSLLLAALLLLLSGDGAVRCDTPANCTYLDLLGTWVFQVGSSGSQRDVNCSVMGPQEKKVVVYLQKLDTAYDDLGNSGHFTIIYNQGFEIVLNDYKWFAFFKDVTDFISHLFMQLGTVGIYDLPHLRNKLAMNRRWG

SEQ ID NO: 197 - protein, 431 aa, Homo sapiens
MSSVCDMSFLQKSCITMVHFCGLLTLHREPVPLKSISVSVNIYEFVAGVSATLNYENEEKVPLEAFFVFPMDEDSAVYSFEALVDGKKIVAELQDKMKARTNYEKAISQGHQAFLLEGDSSSRDVFSCNVGNLQPGSKAAVTLKYVQELPLEADGALRFVLPAVLNPRYQFSGSSKDSCLNVKTPIVPVEDLPYTLSMVATIDSQHGIEKVQSNCPLSPTEYLGEDKTSAQVSLAAGHKFDRDVELLIYYNEVHTPSVVLEMGMPNMKPGHLMGDPSAMVSFYPNIPEDQPSNTCGEFIFLMDRSGSMQSPMSSQDTSQLRIQAAKETLILLLKSLPIGCYFNIYGFGSSYEACFPESVKYTQQTMEEALGRVKLMQADLGGTEILAPLQNIYRGPSIPGHPLQLFVFTDGEVTDTFSVIKEVRINRQKHR

SEQ ID NO: 198 - protein, 786 aa, Homo sapiens
MVHFCGLLTLHREPVPLKSISVSVNIYEFVAGVSATLNYENEEKVPLEAFFVFPMDEDSAVYSFEALVDGKKIVAELQDKMKARTNYEKAISQGHQAFLLEGDSSSRDVFSCNVGNLQPGSKAAVTLKYVQELPLEADGALRFVLPAVLNPRYQFSGSSKDSCLNVKTPIVPVEDLPYTLSMVATIDSQHGIEKVQSNCPLSPTEYLGEDKTSAQVSLAAGHKFDRDVELLIYYNEVHTPSVVLEMGMPNMKPGHLMGDPSAMVSFYPNIPEDQPSNTCGEFIFLMDRSGSMQSPMSSQDTSQLRIQAAKETLILLLKSLPIGCYFNIYGFGSSYEACFPESVKYTQQTMEEALGRVKLMQADLGGTEILAPLQNIYRGPSIPGHPLQLFVFTDGEVTDTFSVIKEVRINRQKHRCFSFGIGEGTSTSLIKGIARASGGTSEFITGKDRMQSKALRTLKRSLQPVVEDVSLSWHLPPGLSAKMLSPEQTVIFRGQRLISYAQLTGRMPAAETTGEVCLKYTLQGKTFEDKVTFPLQPKPDVNLTIHRLAAKSLLQTKDMGLRETPASDKKDALNLSLESGVISSFTAFIAINKELNKPVQGPLAHRDVPRPILLGASAPLKIKCQSGFRKALHSDRPPSASQPRGELMCYKAKTFQMDDYSLCGLISHKDQHSPGFGENHLVQLIYHQNANGSWDLNEDLAKILGMSLEEIMAAQPAELVDSSGWATILAVIWLHSNGKDLKCEWELLERKAVAWMRAHAGSTMPSVVKAAITFLKSSVDPAIFAF

SEQ ID NO: 199 - protein, 786 aa, Homo sapiens; identical to SEQ ID NO: 198

SEQ ID NO: 200 - protein, 327 aa, Homo sapiens
MLSLCICGTAITSQYLAERYKVNTPMLQSFINYCLLFLIYTVMLAFRSGSDNLLVILKRKWWKYILLGLADVEANYVIVRAYQYTTLTSVQLLDCFGIPVLMALSWFILHARYRVIHFIAVAVCLLGVGTMVGADILAGREDNSGSDVLIGDILVLLGASLYAISNVCEEYIVKKLSRQEFLGMVGLFGTIISGIQLLIVEYKDIASIHWDWKIALLFVAFALCMFCLYSFMPLVIKVTSATSVNLGILTADLYSLFVGLFLFGYKFSGLYILSFTVIMVGFILYCSTPTRTAEPAESSVPPVTSIGIDNLGLKLEENLQETHSAVL

SEQ ID NO: 201 - protein, 357 aa, Homo sapiens
MEADSPAGPGAPEPLAEGAAAEFSSLLRRIKGKLFTWNILKTIALGQMLSLCICGTAITSQYLAERYKVNTPMLQSFINYCLLFLIYTVMLAFRSGSDNLLVILKRKWWKYILLGLADVEANYVIVRAYQYTTLTSVQLLDCFGIPVLMALSWFILHARYRVIHFIAVAVCLLGVGTMVGADILAGREDNSGSDVLIGDILVLLGASLYAISNVCEEYIVKKLSRQEFLGMVGLFGTIISGIQLLIVEYKDIASIHWDWKIALLFVAFALCMFCLYSFMPLVIKVTSATSVNLGILTADLYSLFVGLFLFGYKDSTSCPSLSSWWGLSCTAPPLLARPSRLKAACLQSPALGLTTWG

SEQ ID NO: 202 - protein, 374 aa, Homo sapiens
MEADSPAGPGAPEPLAEGAAAEFSSLLRRIKGKLFTWNILKTIALGQMLSLCICGTAITSQYLAERYKVNTPMLQSFINYCLLFLIYTVMLAFRSGSDNLLVILKRKWWKYILLGLADVEANYVIVRAYQYTTLTSVQLLDCFGIPVLMALSWFILHARYRVIHFIAVAVCLLGVGTMVGADILAGREDNSGSDVLIGDILVLLGASLYAISNVCEEYIVKKLSRQEFLGMVGLFGTIISGIQLLIVEYKDIASIHWDWKIALLFVAFALCMFCLYSFMPLVIKVTSATSVNLGILTADLYSLFVGLFLFGYKFSGLYILSFTVIMVGFILYCSTPTRTAEPAESSVPPVTSIGIDNLGLKLEENLQETHSAVL

SEQ ID NO: 203 - protein, 265 aa, Homo sapiens
MEADSPAGPGAPEPLAEGAAAEFSSLLRRIKGKLFTWNILKTIALGQMLSLCICGTAITSQYLAERYKVNTPMLQSFINYCLLFLIYTVMLAFRSGSDNLLVILKRKWWKYILLGLADVEANYVIVRAYQYTTLTSVQLLDCFGIPVLMALSWFILHARYRVIHFIAVAVCLLGVGTMVGADILAGREDNSGSDVLIGDILVLLGASLYAISNVCEEYIVKKLSRQEFLGMVGLFGTIISGIQLPAVRGICPVYVLPVQLHAIGD

SEQ ID NO: 204 - protein, 49 aa, Homo sapiens
MEAMAASTSLPDPGDFDRNVPRICGVCGDRATGFHFNAMTCEGCKGFFR

SEQ ID NO: 205 - protein, 1093 aa, Homo sapiens
MSQPGIPASGGAPASLQAQNGAALASGSPYTNGPVQNALLSSQESVSQGYNFQLPGSYPHPIPAKTLNPVSGQSNYGGSQGSGQTLNRPPVASNPVTPSLHSGPAPRMPLPASQNPATTPMPSSSFLPEANLPPPLNWQYNYPSTASQTNHCPRASSQPTVSGNTSLTTNHQYVSSGYPSLQNSFIKSGPSVPPLVNPPLPTTFQPGAPHGPPPAGGPPPVRALTPLTSSYRDVPQPLFNSAVNQEGITSNTNNGSMVVHSSYDEIEGGGLLATPQLTNKNPKMSRSVGYSYPSLPPGYQNTTPPGATGVPPSSLNYPSGPQAFTQTPLGANHLTTSMSGLSLQPEGLRVVNLLQERNMLPSTPLKPPVPNLHEDIQKLNCNPELFRCTLTSIPQTQALLNKAKLPLGLLLHPFKDLVQLPVVTSSTIVRCRSCRTYINPFVSFLDQRRWKCNLCYRVNDVPEEFLYNPLTRVYGEPHRRPEVQNATIEFMAPSEYMLRPPQPPVYLFVFDVSHNAVETGYLNSVCQSLLDNLDLLPGNTRTKIGFITFDSTIHFYGLQESLSQPQMLIVSDIEDVFIPMPENLLVNLNESKELVQDLLKTLPQMFTKTLETQSALGPALQAAFKLMSPTGGRMSVFQTQLPTLGVGALKPREEPNHRSSAKDIHMTPSTDFYKKLALDCSGQQVAVDLFLLSGQYSDLASLGCISRYSAGSVYYYPSYHHQHNPVQVQKLQKELQRYLTRKIGFEAVMRIRCTKGLSIHTFHGNFFVRSTDLLSLPNVNPDAGYAVQMSVEESLTDTQLVSFQSALLYTSSKGERRIRVHTLCLPVVSTLNDVFLGADVQAISGLLANMAVDRSMTASLSDARDALVNAVIDSLSAYRSSVLSNQQPGLMVPFSLRLFPLFVLALLKQKSFQTGTNARLDERIFAMCQVKNQPLVYLMLTTHPSLYRVDNLSDEGALNISDRTIPQPPILQLSVEKLSRDGAFLMDAGSVLMLWVGKNCTQNFLSQVLGVQNYASIPQPMTDLPELDTPESARIIAFISWLREQRPFFPILYVIRDESPMKANFLQNMIEDRTESALSYYEFLLHIQQQVNK

SEQ ID NO: 206 - protein, 398 aa, Homo sapiens
MLRLYVLVMGVSAFTLQPAAHTGAARSCRFRGRHYKREFRLEGEPVALRCPQVPYWLWASVSPRINLTWHKNDSARTVPGEEETRMWAQDGALWLLPALQEDSGTYVCTTRNASYCDKMSIELRVFENTDAFLPFISYPQILTLSTSGVLVCPDLSEFTRDKTDVKIQWYKDSLLLDKDNEKFLSVRGTTHLLVHDVALEDAGYYRCVLTFAHEGQQYNITRSIELRIKKKKEETIPVIISPLKTISASLGSRLTIPCKVFLGTGTPLTTMLWWTANDTHIESAYPGGRVTEGPRQEYSENNENYIEVPLIFDPVTREDLHMDFKCVVHNTLSFQTLRTTVKEASSTFSWGIVLAPLSLAFLVLGGIWMHRRCKHRTGKADGLTVLWPHHQDFQSYPK

SEQ ID NO: 207 - protein, 398 aa, Homo sapiens; identical to SEQ ID NO: 206

SEQ ID NO: 208 - protein, 296 aa, Homo sapiens
MLRLYVLVMGVSAFTLQPAAHTGAARSCRFRGRHYKREFRLEGEPVALRCPQVPYWLWASVSPRINLTWHKNDSARTVPGEEETRMWAQDGALWLLPALQEDSGTYVCTTRNASYCDKMSIELRVFENTDAFLPFISYPQILTLSTSGVLVCPDLSEFTRDKTDVKIQWYKDSLLLDKDNEKFLSVRGTTHLLVHDVALEDAGYYRCVLTFAHEGQQYNITRSIELRIKKKKEETIPVIISPLKTISASLGSRLTIPCKVFLGTGTPLTTMLWWTANDTHIESAYPGGRVTEGPRQ

SEQ ID NO: 209 - protein, 229 aa, Homo sapiens
MLRLYVLVMGVSAFTLQPAAHTGAARSCRFRGRHYKREFRLEGEPVALRCPQVPYWLWASVSPRINLTWHKNDSARTVPGEEETRMWAQDGALWLLPALQEDSGTYVCTTRNASYCDKMSIELRVFENTDAFLPFISYPQILTLSTSGVLVCPDLSEFTRDKTDVKIQWYKDSLLLDKDNEKFLSVRGTTHLLVHDVALEDAGYYRCVLTFAHEGQQYNITRSIELRIK

SEQ ID NO: 210 - protein, 447 aa, Homo sapiens
MKVLLRLICFIALLISSLEADKCKEREEKIILVSSANEIDVRPCPLNPNEHKGTITWYKDDSKTPVSTEQASRIHQHKEKLWFVPAKVEDSGHYYCVVRNSSYCLRIKISAKFVENEPNLCYNAQAIFKQKLPVAGDGGLVCPYMEFFKNENNELPKLQWYKDCKPLLLDNIHFSGVKDRLIVMNVAEKHRGNYTCHASYTYLGKQYPITRVIEFITLEENKPTRPVIVSPANETMEVDLGSQIQLICNVTGQLSDIAYWKWNGSVIDEDDPVLGEDYYSVENPANKRRSTLITVLNISEIESRFYKHPFTCFAKNTHGIDAAYIQLIYPVTNFQKHMIGICVTLTVIIVCSVFIYKIFKIDIVLWYRDSCYDFLPIKASDGKTYDAYILYPKTVGEGSTSDCDIFVFKVLPEVLEKQCGYKLFIYGRDDYVGEGMCVMEQSKGLLL

SEQ ID NO: 211 - protein, 447 aa, Homo sapiens; identical to SEQ ID NO: 210

SEQ ID NO: 212 - protein, 173 aa, Homo sapiens
MKVLLRLICFIALLISSLEADKCKEREEKIILVSSANEIDVRPCPLNPNEHKGTITWYKDDSKTPVSTEQASRIHQHKEKLWFVPAKVEDSGHYYCVVRNSSYCLRIKISAKFVENEPNLCYNAQAIFKQKLPVAGDGGLVCPYMEFFKNENNELPKLQWYKVAVSRVWATAL

SEQ ID NO: 213 - protein, 538 aa, Homo sapiens
MKVLLRLICFIALLISSLEADKCKEREEKIILVSSANEIDVRPCPLNPNEHKGTITWYKDDSKTPVSTEQASRIHQHKEKLWFVPAKVEDSGHYYCVVRNSSYCLRIKISAKFVENEPNLCYNAQAIFKQKLPVAGDGGLVCPYMEFFKNENNELPKLQWYKDCKPLLLDNIHFSGVKDRLIVMNVAEKHRGNYTCHASYTYLGKQYPITRVIEFITLEENKPTRPVIVSPANETMEVDLGSQIQLICNVTGQLSDIAYWKWNGSVIDEDDPVLGEDYYSVENPANKRRSTLITVLNISEIESRFYKHPFTCFAKNTHGIDAAYIQLIYPVTNFQKHMIGICVTLTVIIVCSVFIYKIFKIDIVLWYRDSCYDFLPIKVLPEVLEKQCGYKLFIYGRDDYVGEDIVEVINENVKKSRRLIIILVRETSGFSWLGGSSEEQIAMYNALVQDGIKVVLLELEKIQDYEKMPESIKFIKQKHGAIRWSGDFTQGPQSAKTRFWKNVRYHMPVQRRSPSSKHQLLSPATKEKLQREAHVPLG

SEQ ID NO: 214 - protein, 569 aa, Homo sapiens
MKVLLRLICFIALLISSLEADKCKEREEKIILVSSANEIDVRPCPLNPNEHKGTITWYKDDSKTPVSTEQASRIHQHKEKLWFVPAKVEDSGHYYCVVRNSSYCLRIKISAKFVENEPNLCYNAQAIFKQKLPVAGDGGLVCPYMEFFKNENNELPKLQWYKDCKPLLLDNIHFSGVKDRLIVMNVAEKHRGNYTCHASYTYLGKQYPITRVIEFITLEENKPTRPVIVSPANETMEVDLGSQIQLICNVTGQLSDIAYWKWNGSVIDEDDPVLGEDYYSVENPANKRRSTLITVLNISEIESRFYKHPFTCFAKNTHGIDAAYIQLIYPVTNFQKHMIGICVTLTVIIVCSVFIYKIFKIDIVLWYRDSCYDFLPIKASDGKTYDAYILYPKTVGEGSTSDCDIFVFKVLPEVLEKQCGYKLFIYGRDDYVGEDIVEVINENVKKSRRLIIILVRETSGFSWLGGSSEEQIAMYNALVQDGIKVVLLELEKIQDYEKMPESIKFIKQKHGAIRWSGDFTQGPQSAKTRFWKNVRYHMPVQRRSPSSKHQLLSPATKEKLQREAHVPLG

SEQ ID NO: 215 - protein, 119 aa, Homo sapiens
MKVLLRLICFIALLISSLEADKCKEREEKIILVSSANEIDVRPCPLNPNEHKGTITWYKDDSKTPVSTEQASRIHQHKEKLWFVPAKVEDSGHYYCVVRIANLYFLTIYTLVESKIGSS

SEQ ID NO: 216 - protein, 124 aa, Homo sapiens
XKCKEREEKIILVSSANEIDVRPCPLNPNEHKGTITWYKDDSKTPVSTEQASRIHQHKEKLWFVPAKVEDSGHYYCVETEDLCALIWSFLKMKIMSYLNYSGIRIANLYFLTIYTLVESKIGSS

SEQ ID NO: 217 - protein, 447 aa, Homo sapiens; identical to SEQ ID NO: 210

SEQ ID NO: 218 - protein, 298 aa, Homo sapiens
MEFFKNENNELPKLQWYKDCKPLLLDNIHFSGVKDRLIVMNVAEKHRGNYTCHASYTYLGKQYPITRVIEFITLEENKPTRPVIVSPANETMEVDLGSQIQLICNVTGQLSDIAYWKWNGSVIDEDDPVLGEDYYSVENPANKRRSTLITVLNISEIESRFYKHPFTCFAKNTHGIDAAYIQLIYPVTNFQKHMIGICVTLTVIIVCSVFIYKIFKIDIVLWYRDSCYDFLPIKASDGKTYDAYILYPKTVGEGSTSDCDIFVFKVLPEVLEKQCGYKLFIYGRDDYVGEDIVEVINE

SEQ ID NO: 219 - protein, 182 aa, Homo sapiens
MKVLLRLICFIALLISSLEADKCKEREEKIILVSSANEIDVRPCPLNPNEHKGTITWYKDDSKTPVSTEQASRIHQHKEKLWFVPAKVEDSGHYYCVVRNSSYCLRIKISAKFVENEPNLCYNAQAIFKQKLPVAGDGGLVCPYMEFFKNENNELPKLQWYKDCKPLLLDNIHFSGVKDRLI

SEQ ID NO: 220 - protein, 114 aa, Homo sapiens
MKVLLRLICFIALLISSLEADKCKEREEKIILVSSANEIDVRPCPLNPNEHKGTITWYKDDSKTPVSTEQASRIHQHKEKLWFVPAKVEDSGHYYCVVRNSSYCLRIKISAKFV

SEQ ID NO: 221 - protein, 106 aa, Homo sapiens
MKVLLRLICFIALLISSLEADKCKEREEKIILVSSANEIDVRPCPLNPNEHKGTITWYKDDSKTPVSTEQASRIHQHKEKLWFVPAKVEDSGHYYCVVRNSSYCLR

SEQ ID NO: 222 - protein, 80 aa, Homo sapiens
MKVLLRLICFIALLISSLEADKCKEREEKIILVSSANEIDVRPCPLNPNEHKGTITWYKDDSKTPVSTEQASRIHQHKEK

SEQ ID NO: 223 - protein, 575 aa, Homo sapiens
MWSLLLCGLSIALPLSVTADGCKDIFMKNEILSASQPFAFNCTFPPITSGEVSVTWYKNSSKIPVSKIIQSRIHQDETWILFLPMEWGDSGVYQCVIKGRDSCHRIHVNLTVFEKHWCDTSIGGLPNLSDEYKQILHLGKDDSLTCHLHFPKSCVLGPIKWYKDCNEIKGERFTVLETRLLVSNVSAEDRGNYACQAILTHSGKQYEVLNGITVSITERAGYGGSVPKIIYPKNHSIEVQLGTTLIVDCNVTDTKDNTNLRCWRVNNTLVDDYYDESKRIREGVETHVSFREHNLYTVNITFLEVKMEDYGLPFMCHAGVSTAYIILQLPAPDFRAYLIGGLIALVAVAVSVVYIYNIFKIDIVLWYRSAFHSTETIVDGKLYDAYVLYPKPHKESQRHAVDALVLNILPEVLERQCGYKLFIFGRDEFPGQAVANVIDENVKLCRRLIVIVVPESLGFGLLKNLSEEQIAVYSALIQDGMKVILIELEKIEDYTVMPESIQYIKQKHGAIRWHGDFTEQSQCMKTKFWKTVRYHMPPRRCRPFPPVQLLQHTPCYRTAGPELGSRRKKCTLTTG

SEQ ID NO: 224 - protein, 172 aa, Homo sapiens
MWSLLLCGLSIALPLSVTADGCKDIFMKNEILSASQPFAFNCTFPPITSGEVSVTWYKNSSKIPVSKIIQSRIHQDETWILFLPMEWGDSGVYQCVIKGRDSCHRIHVNLTVFEKHWCDTSIGGLPNLSDEYKQILHLGKDDSLTCHLHFPKSCVLGPIKWYKDCNEIKGER

SEQ ID NO: 225 - protein, 457 aa, Homo sapiens
MTGLVSLSYFPLSTRSCALQSCSPVWGCGPCCSAGCPSPFHCLSQQDCNEIKGERFTVLETRLLVSNVSAEDRGNYACQAILTHSGKQYEVLNGITVSIKRAGYGGSVPKIIYPKNHSIEVQLGTTLIVDCNVTDTKDNTNLRCWRVNNTLVDDYYDESKRIREGVETHVSFREHNLYTVNITFLEVKMEDYGLPFMCHAGVSTAYIILQLPAPDFRAYLIGGLIALVAVAVSVVYIYNIFKIDIVLWYRSAFHSTETIVDGKLYDAYVLYPKPHKESQRHAVDALVLNILPEVLERQCGYKLFIFGRDEFPGQAVANVIDENVKLCRRLIVIVVPESLGFGLLKNLSEEQIAVYSALIQDGMKVILIELEKIEDYTVMPESIQYIKQKHGAIRWHGDFTEQSQCMKTKFWKTVRYHMPPRRCRPFPPVQLLQHTPCYRTAGPELGSRRKKCTLTTG

SEQ ID NO: 226 - protein, 556 aa, Homo sapiens
MGFWILAILTILMYSTAAKFSKQSWGLENEALIVRCPRQGKPSYTVDWYYSQTNKSIPTQERNRVFASGQLLKFLPAAVADSGIYTCIVRSPTFNRTGYANVTIYKKQSDCNVPDYLMYSTVSGSEKNSKIYCPTIDLYNWTAPLEWFKNCQALQGSRYRAHKSFLVIDNVMTEDAGDYTCKFIHNENGANYSVTATRSFTVKDEQGFSLFPVIGAPAQNEIKEVEIGKNANLTCSACFGKGTQFLAAVLWQLNGTKITDFGEPRIQQEEGQNQSFSNGLACLDMVLRIADVKEEDLLLQYDCLALNLHGLRRHTVRLSRKNPIDHHSIYCIIAVCSVFLMLINVLVIILKMFWIEATLLWRDIAKPYKTRNDGKLYDAYVVYPRNYKSSTDGASRVEHFVHQILPDVLENKCGYTLCIYGRDMLPGEDVVTAVETNIRKSRRHIFILTPQITHNKEFAYEQEVALHCALIQNDAKVILIEMEALSELDMLQAEALQDSLQHLMKVQGTIKWREDHIANKRSLNSKFWKHVRYQMPVPSKIPRKASSLTPLAAQKQ

SEQ ID NO: 227 - protein, 328 aa, Homo sapiens
MGFWILAILTILMYSTAAKFSKQSWGLENEALIVRCPRQGKPSYTVDWYYSQTNKSIPTQERNRVFASGQLLKFLPAAVADSGIYTCIVRSPTFNRTGYANVTIYKKQSDCNVPDYLMYSTVSGSEKNSKIYCPTIDLYNWTAPLEWFKNCQALQGSRYRAHKSFLVIDNVMTEDAGDYTCKFIHNENGANYSVTATRSFTVKDEQGFSLFPVIGAPAQNEIKEVEIGKNANLTCSACFGKGTQFLAAVLWQLNGTKITDFGEPRIQQEEGQNQSFSNGLACLDMVLRIADVKEEDLLLQYDCLALNLHGLRRHTVRLSRKNPSKECF

SEQ ID NO: 228 - protein, 211 aa, Homo sapiens
MYSTVSGSEKNSKIYCPTIDLYNWTAPLEWFKNCQALQGSRYRAHKSFLVIDNVMTEDAGDYTCKFIHNENGANYSVTATRSFTVKDEQGFSLFPVIGAPAQNEIKEVEIGKNANLTCSACFGKGTQFLAAVLWQLNGTKITDFGEPRIQQEEGQNQSFSNGLACLDMVLRIADVKEEDLLLQYDCLALNLHGLRRHTVRLSRKNPSKECF

SEQ ID NO: 229 - protein, 314 aa, Homo sapiens
MGFWILAILTILMYSTAAKFSKQSWGLENEALIVRCPRQGKPSYTVDWYYSQTNKSIPTQERNRVFASGQLLKFLPAAVADSGIYTCIVRSPTFNRTGYANVTIYKKQSDCNVPDYLMYSTVSGSEKNSKIYCPTIDLYNWTAPLEWFKNCQALQGSRYRAHKSFLVIDNVMTEDAGDYTCKFIHNENGANYSVTATRSFTVKDEQGFSLFPVIGAPAQNEIKEVEIGTQFLAAVLWQLNGTKITDFGEPRIQQEEGQNQSFSNGLACLDMVLRIADVKEEDLLLQYDCLALNLHGLRRHTVRLSRKNPSKECF

SEQ ID NO: 230 - protein, 259 aa, Homo sapiens
MGFWILAILTILMYSTAAKFSKQSWGLENEALIVRCPRQGKPSYTVDWYYSQTNKSIPTQERNRVFASGQLLKFLPAAVADSGIYTCIVRSPTFNRTGYANVTIYKKQSDCNVPDYLMYSTVSGSEKNSKIYCPTIDLYNWTAPLEWFKNCQALQGSRYRAHKSFLVIDNVMTEDAGDYTCKFIHNENGANYSVTATRSFTVKVWCQSFCKLKKSLIFSNTHWIQSLMRGFVMVYYGVHKCCRVVFNLCLQYFQHHQWP

SEQ ID NO: 231 - protein, 38 aa, Homo sapiens
MGFWILAILTILMYSTAAKFSKQSWGLENEALIVRCPR

SEQ ID NO: 232 - protein, 425 aa, Homo sapiens
MVSKALLRLVSAVNRRRMKLLLGIALLAYVASVWGNFVNMSFLLNRSIQENGELKIESKIEEMVEPLREKIRDLEKSFTQKYPPVKFLSEKDRKRILITGGAGFVGSHLTDKLMMDGHEVTVVDNFFTGRKRNVEHWIGHENFELINHDVVEPLYIEVDQIYHLASPASPPNYMYNPIKTLKTNTIGTLNMLGLAKRVGARLLLASTSEVYGDPEVHPQSEDYWGHVNPIGPRACYDEGKRVAETMCYAYMKQEGVEVRVARIFNTFGPRMHMNDGRVVSNFILQALQGEPLTVYGSGSQTRAFQYVSDLVNGLVALMNSNVSSPVNLGNPEEHTILEFAQLIKNLVGSGSEIQFLSEAQDDPQKRKPDIKKAKLMLGWEPVVPLEEGLNKAIHYFRKELEYQANNQYIPKPKPARIKKGRTRHS

SEQ ID NO: 233 - protein, 420 aa, Homo sapiens
MVSKALLRLVSAVNRRRMKLLLGIALLAYVASVWGNFVNMRSIQENGELKIESKIEEMVEPLREKIRDLEKSFTQKYPPVKFLSEKDRKRILITGGAGFVGSHLTDKLMMDGHEVTVVDNFFTGRKRNVEHWIGHENFELINHDVVEPLYIEVDQIYHLASPASPPNYMYNPIKTLKTNTIGTLNMLGLAKRVGARLLLASTSEVYGDPEVHPQSEDYWGHVNPIGPRACYDEGKRVAETMCYAYMKQEGVEVRVARIFNTFGPRMHMNDGRVVSNFILQALQGEPLTVYGSGSQTRAFQYVSDLVNGLVALMNSNVSSPVNLGNPEEHTILEFAQLIKNLVGSGSEIQFLSEAQDDPQKRKPDIKKAKLMLGWEPVVPLEEGLNKAIHYFRKELEYQANNQYIPKPKPARIKKGRTRHS

SEQ ID NO: 234 - protein, 171 aa, Homo sapiens [sequence omitted]

SEQ ID NO: 235 - protein, 190 aa, Homo sapiens
MVEPLREKIRDLEKSFTQKYPPVKFLSEKDRKRILITGGAGFVGSHLTDKLMMDGHEVTVVDNFFTGRKRNVEHWIGHENFELINHDVVEPLYIEVDQIYHLASPASPPNYMYNPIKTLKTNTIGTLNMLGLAKRVGARLLLASTSEVYGDPEVHPQSEDYWGHVNPIGPRACYDEGKRVAETMCYAYMK

SEQ ID NO: 236 - protein, 210 aa, Homo sapiens
MAVKVQTTKRGDPHELRNIFLQYASTEVDGERYMTPEDFVQRYLGLYNDPNSNPKIVQLLAGVADQTKDGLISYQEFLAFESVLCAPDSMFIVAFQLFDKSGNGEVTFGGVLWPHRWYSSSFQAHSWNRHGGSLGEECQPRSEKMSKKFLDRLLFIIISLLTGIVNLSDCILGITGRSILTTQNSRSFSRSCNWNMQDKPLHSKTKAKVA

SEQ ID NO: 237 - protein, 870 aa, Homo sapiens
MGVKKKKEMQVAALTICHQDLETLKSFADVEGKNLASLLLHCVQLTDGVSQIHYIKQIVPLLEKADKNGMCDPTIQSCLDILAGIYLSLSLKNPLKKVLASSLNSLPDFFLPEAMHRFTSRLQEELNTTDLYSYRKVTDNISSCMENFNLGRASVNNLLKNVLHFLQKSLIEILEENRKCAGNHIIQTQLMNDLLVGIRVSMMLVQKVQDFQGNLWKTSDSPIWQNMCGLLSIFTKVLSDDDLLQTVQSTSGLAIILFIKTMFHPSEKIPHLISSVLLRSVDCTSVPEWFMSSCRSLCCGDISQSAVLFLCQGTLAMLDWQNGSMGRSGEALLLDTAHVLFTLSSQIKEPTLEMFLSRILASWTNSAIQVLESSSPSLTDSLNGNSSIVGRLLEYVYTHWEHPLDALRHQTKIMFKNLLQMHRLTVEGADFVPDPFFVELTESLLRLEWHIKGKYTCLGCLVECIGVEHILAIDKTIPSQILEVMGDQSLVPYASDLLETMFRNHKSHLKSQTAESSWIDQWHETWVSPLLFILCEGNLDQKSYVIDYYLPKLLSYSPESLQYMVKILQTSIDAKTGQEQSFPSLGSCNSRGALGALMACLRIARAHGHLQSATDTWENLVSDARIKQGLIHQHCQVRIDTLGLLCESNRSTEIVSMEEMQWIQFFITYNLNSQSPGVRQQICSLLKKLFCRIQESSQVLYKLEQSKSKREPENELTKQHPSVSLQQYKNFMSSICNSLFEALFPGSSYSTRFSALTILGSIAEVFHVPEGKCLIMLGEVIFVFFQNILILHFALFEVSVLLPFFEVSFLPYNSCLYIMKFASKGQKRKKLSPFSLIIICQPQSIIFMDYAYYGKSSSYYLTNLFVAVLR

SEQ ID NO: 238 - protein, 906 aa, Homo sapiens
MICQKFCVVLLHWEFIYVITAFNLSYPITPWRFKLSCMPPNSTYDYFLLPAGLSKNTSNSNGHYETAVEPKFNSSGTHFSNLSKTTFHCCFRSEQDRNCSLCADNIEGKTFVSTVNSLVFQQIDANWNIQCWLKGDLKLFICYVESLFKNLFRNYNYKVHLLYVLPEVLEDSPLVPQKGSFQMVHCNCSVHECCECLVPVPTAKLNDTLLMCLKITSGGVIFQSPLMSVQPINMVKPDPPLGLHMEITDDGNLKISWSSPPLVPFPLQYQVKYSENSTTVIREADKIVSATSLLVDSILPGSSYEVQVRGKRLDGPGIWSDWSTPRVFTTQDVIYFPPKILTSVGSNVSFHCIYKKENKIVPSKEIVWWMNLAEKIPQSQYDVVSDHVSKVTFFNLNETKPRGKFTYDAVYCCNEHECHHRYAELYVIDVNINISCETDGYLTKMTCRWSTSTIQSLAESTLQLRYHRSSLYCSDIPSIHPISEPKDCYLQSDGFYECIFQPIFLLSGYTMWIRINHSLGSLDSPPTCVLPDSVVKPLPPSSVKAEITINIGLLKISWEKPVFPENNLQFQIRYGLSGKEVQWKMYEVYDAKSKSVSLPVPDLCAVYAVQVRCKRLDGLGYWSNWSNPAYTVVMDIKVPMRGPEFWRIINGDTMKKEKNVTLLWKPLMKNDSLCSVQRYVINHHTSCNGTWSEDVGNHTKFTFLWTEQAHTVTVLAINSIGASVANFNLTFSWPMSKVNIVQSLSAYPLNSSCVIVSWILSPSDYKLMYFIIEWKNLNEDGEIKWLRISSSVKKYYIHDHFIPIEKYQFSLYPIFMEGVGKPKIINSFTQDDIEKHQSDAGLYVIVPVIISSSILLLGTLLISHQRMKKLFWEDVPNPKNCSWAQGLNFQKKMPGTKELLGGGWLT

SEQ ID NO: 239 - protein, 1165 aa, Homo sapiens
MICQKFCVVLLHWEFIYVITAFNLSYPITPWRFKLSCMPPNSTYDYFLLPAGLSKNTSNSNGHYETAVEPKFNSSGTHFSNLSKTTFHCCFRSEQDRNCSLCADNIEGKTFVSTVNSLVFQQIDANWNIQCWLKGDLKLFICYVESLFKNLFRNYNYKVHLLYVLPEVLEDSPLVPQKGSFQMVHCNCSVHECCECLVPVPTAKLNDTLLMCLKITSGGVIFQSPLMSVQPINMVKPDPPLGLHMEITDDGNLKISWSSPPLVPFPLQYQVKYSENSTTVIREADKIVSATSLLVDSILPGSSYEVQVRGKRLDGPGIWSDWSTPRVFTTQDVIYFPPKILTSVGSNVSFHCIYKKENKIVPSKEIVWWMNLAEKIPQSQYDVVSDHVSKVTFFNLNETKPRGKFTYDAVYCCNEHECHHRYAELYVIDVNINISCETDGYLTKMTCRWSTSTIQSLAESTLQLRYHRSSLYCSDIPSIHPISEPKDCYLQSDGFYECIFQPIFLLSGYTMWIRINHSLGSLDSPPTCVLPDSVVKPLPPSSVKAEITINIGLLKISWEKPVFPENNLQFQIRYGLSGKEVQWKMYEVYDAKSKSVSLPVPDLCAVYAVQVRCKRLDGLGYWSNWSNPAYTVVMDIKVPMRGPEFWRIINGDTMKKEKNVTLLWKPLMKNDSLCSVQRYVINHHTSCNGTWSEDVGNHTKFTFLWTEQAHTVTVLAINSIGASVANFNLTFSWPMSKVNIVQSLSAYPLNSSCVIVSWILSPSDYKLMYFIIEWKNLNEDGEIKWLRISSSVKKYYIHDHFIPIEKYQFSLYPIFMEGVGKPKIINSFTQDDIEKHQSDAGLYVIVPVIISSSILLLGTLLISHQRMKKLFWEDVPNPKNCSWAQGLNFQKPETFEHLFIKHTASVTCGPLLLEPETISEDISVDTSWKNKDEMMPTTVVSLLSTTDLEKGSVCISDQFNSVNFSEAEGTEVTYEDESQRQPFVKYATLISNSKPSETGEEQGLINSSVTKCFSSKNSPLKDSFSNSSWEIEAQAFFILSDQHPNIISPHLTFSEGLDELLKLEGNFPEENNDKKSIYYLGVTSIKKRESGVLLTDKSRVSCPFPAPCLFTDIRVLQDSCSHFVENNINLGTSSKKTFASYMPQFQTCSTQTHKIMENKMCDLTV

SEQ ID NO: 240 - protein, 906 aa, Homo sapiens; identical to SEQ ID NO: 238

SEQ ID NO: 241 - protein, 958 aa, Homo sapiens
MICQKFCVVLLHWEFIYVITAFNLSYPITPWRFKLSCMPPNSTYDYFLLPAGLSKNTSNSNGHYETAVEPKFNSSGTHFSNLSKTTFHCCFRSEQDRNCSLCADNIEGKTFVSTVNSLVFQQIDANWNIQCWLKGDLKLFICYVESLFKNLFRNYNYKVHLLYVLPEVLEDSPLVPQKGSFQMVHCNCSVHECCECLVPVPTAKLNDTLLMCLKITSGGVIFQSPLMSVQPINMVKPDPPLGLHMEITDDGNLKISWSSPPLVPFPLQYQVKYSENSTTVIREADKIVSATSLLVDSILPGSSYEVQVRGKRLDGPGIWSDWSTPRVFTTQDVIYFPPKILTSVGSNVSFHCIYKKENKIVPSKEIVWWMNLAEKIPQSQYDVVSDHVSKVTFFNLNETKPRGKFTYDAVYCCNEHECHHRYAELYVIDVNINISCETDGYLTKMTCRWSTSTIQSLAESTLQLRYHRSSLYCSDIPSIHPISEPKDCYLQSDGFYECIFQPIFLLSGYTMWIRINHSLGSLDSPPTCVLPDSVVKPLPPSSVKAEITINIGLLKISWEKPVFPENNLQFQIRYGLSGKEVQWKMYEVYDAKSKSVSLPVPDLCAVYAVQVRCKRLDGLGYWSNWSNPAYTVVMDIKVPMRGPEFWRIINGDTMKKEKNVTLLWKPLMKNDSLCSVQRYVINHHTSCNGTWSEDVGNHTKFTFLWTEQAHTVTVLAINSIGASVANFNLTFSWPMSKVNIVQSLSAYPLNSSCVIVSWILSPSDYKLMYFIIEWKNLNEDGEIKWLRISSSVKKYYIHDHFIPIEKYQFSLYPIFMEGVGKPKIINSFTQDDIEKHQSDAGLYVIVPVIISSSILLLGTLLISHQRMKKLFWEDVPNPKNCSWAQGLNFQKMLEGSMFVKSHHHSLISSTQGHKHCGRPQGPLHRKTRDLCSLVYLLTLPPLLSYDPAKSPSVRNTQE

SEQ ID NO: 242 - protein, 896 aa, Homo sapiens
MICQKFCVVLLHWEFIYVITAFNLSYPITPWRFKLSCMPPNSTYDYFLLPAGLSKNTSNSNGHYETAVEPKFNSSGTHFSNLSKTTFHCCFRSEQDRNCSLCADNIEGKTFVSTVNSLVFQQIDANWNIQCWLKGDLKLFICYVESLFKNLFRNYNYKVHLLYVLPEVLEDSPLVPQKGSFQMVHCNCSVHECCECLVPVPTAKLNDTLLMCLKITSGGVIFQSPLMSVQPINMVKPDPPLGLHMEITDDGNLKISWSSPPLVPFPLQYQVKYSENSTTVIREADKIVSATSLLVDSILPGSSYEVQVRGKRLDGPGIWSDWSTPRVFTTQDVIYFPPKILTSVGSNVSFHCIYKKENKIVPSKEIVWWMNLAEKIPQSQYDVVSDHVSKVTFFNLNETKPRGKFTYDAVYCCNEHECHHRYAELYVIDVNINISCETDGYLTKMTCRWSTSTIQSLAESTLQLRYHRSSLYCSDIPSIHPISEPKDCYLQSDGFYECIFQPIFLLSGYTMWIRINHSLGSLDSPPTCVLPDSVVKPLPPSSVKAEITINIGLLKISWEKPVFPENNLQFQIRYGLSGKEVQWKMYEVYDAKSKSVSLPVPDLCAVYAVQVRCKRLDGLGYWSNWSNPAYTVVMDIKVPMRGPEFWRIINGDTMKKEKNVTLLWKPLMKNDSLCSVQRYVINHHTSCNGTWSEDVGNHTKFTFLWTEQAHTVTVLAINSIGASVANFNLTFSWPMSKVNIVQSLSAYPLNSSCVIVSWILSPSDYKLMYFIIEWKNLNEDGEIKWLRISSSVKKYYIHDHFIPIEKYQFSLYPIFMEGVGKPKIINSFTQDDIEKHQSDAGLYVIVPVIISSSILLLGTLLISHQRMKKLFWEDVPNPKNCSWAQGLNFQKRTDIL

SEQ ID NO: 243 - protein, 232 aa, Homo sapiens
MMPTTVVSLLSTTDLEKGSVCISDQFNSVNFSEAEGTEVTYEDESQRQPFVKYATLISNSKPSETGEEQGLINSSVTKCFSSKNSPLKDSFSNSSWEIEAQAFFILSDQHPNIISPHLTFSEGLDELLKLEGNFPEENNDKKSIYYLGVTSIKKRESGVLLTDKSRVSCPFPAPCLFTDIRVLQDSCSHFVENNINLGTSSKKTFASYMPQFQTCSTQTHKIMENKMCDLTV

SEQ ID NO: 244 - protein, 896 aa, Homo sapiens; identical to SEQ ID NO: 242

SEQ ID NO: 245 - protein, 214 aa, Homo sapiens
MGLRDWLRTVCCCCGCECLEERALPEKEPLVSDNNPYSSFGATLVRDDEKNLWSMPHDVSHTEADDDRTLYNLIVIRNQQAKDSEEWQKLNYDIHTLRQVRREVRNRWKCILEDLGFQKEADSLLSVTKLSTISDSKNTRKAREMLLKLAEETNIFPTSWELSERYLFVVDRLIALDAAEEFFKLARRTYPKKPGVPCLADGQKELHYLPFPSP

SEQ ID NO: 246 - protein, 214 aa, Homo sapiens; identical to SEQ ID NO: 245

SEQ ID NO: 247 - protein, 526 aa, Homo sapiens
MVNEGPNQEESDDTPVPESALQADPSVSVHPSVSVHPSVSINPSVSVHPSSSAHPSALAQPSGLAHPSSSGPEDLSVIKVSRRRWAVVLVFSCYSMCNSFQWIQYGSINNIFMHFYGVSAFAIDWLSMCYMLTYIPLLLPVAWLLEKFGLRTIALTGSALNCLGAWVKLGSLKPHLFPVTVVGQLICSVAQVFILGMPSRIASVWFGANEVSTACSVAVFGNQLGIAIGFLVPPVLVPNIEDRDELAYHISIMFYIIGGVATLLLILVIIVFKEKPKYPPSRAQSLSYALTSPDASYLGSIARLFKNLNFVLLVITYGLNAGAFYALSTLLNRMVIWHYPGEEVNAGRIGLTIVIAGMLGAVISGIWLDRSKTYKETTLVVYIMTLVGMVVYTFTLNLGHLWVVFITAGTMGFFMTGYLPLGFEFAVELTYPESEGISSGLLNISAQVFGIIFTISQGQIIDNYGTKPGNIFLCVFLTLGAALTAFIKADLRRQKANKETLENKLQEEEEESNTSKVPTAVSEDHL

SEQ ID NO: 248 - protein, 321 aa, Homo sapiens
MSADNSSTICVCRSVRQELGIAIGFLVPPVLVPNIEDRDELAYHISIMFYIIGGVATLLLILVIIVFKEKPKYPPSRAQSLSYALTSPDASYLGSIARLFKNLNFVLLVITYGLNAGAFYALSTLLNRMVIWHYPGEEVNAGRIGLTIVIAGMLGAVISGIWLDRSKTYKETTLVVYIMTLVGMVVYTFTLNLGHLWVVFITAGTMGFFMTGYLPLGFEFAVELTYPESEGISSGLLNISAQVFGIIFTISQGQIIDNYGTKPGNIFLCVFLTLGAALTAFIKADLRRQKANKETLENKLQEEEEESNTSKVPTAVSEDHL

SEQ ID NO: 249 - protein, 203 aa, Homo sapiens
MFYIIGGVATLLLILVIIGLNAGAFYALSTLLNRMVIWHYPGEEVNAGRIGLTIVIAGMLGAVISGIWLDRSKTYKETTLVVYIMTLVGMVVYTFTLNLGHLWVVFITAGTMGFFMTGYLPLGFEFAVELTYPESEGISSGLLNISAQVFGIIFTISQGQIIDNYGTKPGNIFLCVFLTLGAALTAFIKADLRRQKANKETLE

SEQ ID NO: 250 - protein, 133 aa, Homo sapiens
MFYIIGGVATLLLILVIIVFKEKPKYPPSRAQSLSYALTSPDASYLGSIARLFKNLNFVLLVITYGLNAGAFYALSTLLNRMVIWHYPGEEVNAGRIGLTIVIAGMLGAVISGIWLDRSKTYKNLCHPNCLIQ

SEQ ID NO: 251 - protein, 203 aa, Homo sapiens
MSPYGISSLGNGLSKWLSLNAGAFYALSTLLNRMVIWHYPGEEVNAGRIGLTIVIAGMLGAVISGIWLDRSKTYKETTLVVYIMTLVGMVVYTFTLNLGHLWVVFITAGTMGFFMTGYLPLGFEFAVELTYPESEGISSGLLNISAQVFGIIFTISQGQIIDNYGTKPGNIFLCVFLTLGAALTAFIKADLRRQKANKETLEN

SEQ ID NO: 252 - protein, 241 aa, Homo sapiens
MCPDLHPSKYVQPPLSSSSSPLKGGQEAFSPLGLNAGAFYALSTLLNRMVIWHYPGEEVNAGRIGLTIVIAGMLGAVISGIWLDRSKTYKETTLVVYIMTLVGMVVYTFTLNLGHLWVVFITAGTMGFFMTGYLPLGFEFAVELTYPESEGISSGLLNISAQVFGIIFTISQGQIIDNYGTKPGNIFLCVFLTLGAALTAFIKADLRRQKANKETLENKLQEEEEESNTSKVPTAVSEDHL

SEQ ID NO: 253 - protein, 88 aa, Homo sapiens
MFYIIGGVATLLLILVIIVFKEKPKYPPSRAQSLSYALTSPDASYLGSIARLFKNLNFVLLVITYGLNAGAFYALSTLLNRMVIWHYP

SEQ ID NO: 254 - protein, 93 aa, Homo sapiens
MFYIIGGVATLLLILVIIVFKEKPKYPPSRAQSLSYALTSPDASYLGSIARLFKNLNFVLLVITYETPRGGGGEQHQQSAHCCVRGSSLRGRW

SEQ ID NO: 255 - protein, 102 aa, Homo sapiens
MTLRCLEPSGNGGEGTRSQWGTAGSAEEPSPQAARLAKALRELGQTGREPIGRDACGRERLPKEAARKRGRGGKQRIRWKYVPCFQGTAESTLAGSWVLGNA

SEQ ID NO: 256 - protein, 46 aa, Homo sapiens
XAPSLQHLCRLTINKCTGAIWGLPLPTRLKDYLEEYKFQPAVFYPG

SEQ ID NO: 257 - protein, 341 aa, Homo sapiens
MNIVVEFFVVTFKVLWAFVLAAARWLVRPKEKSVAGQVCLITGAGSGLGRLFALEFARRRALLVLWDINTQSNEETAGMVRHIYRDLEAADAAALQAGNGEEEILPHCNLQVFTYTCDVGKRENVYLTAERVRKEVGEVSVLVNNAGVVSGHHLLECPDELIERTMMVNCHAHFWTTKAFLPTMLEINHGHIVTVASSLGLFSTAGVEDYCASKFGVVGFHESLSHELKAAEKDGIKTTLVCPYLVDTGMFRGCRIRKEIEPFLPPLKPDYCVKQAMKAILTDQPMICTPRLMYIVTFMKSILPFEAVVCMYRFLGADKCMYPFIAQRKQATNNNEAKNGI

SEQ ID NO: 258 - protein, 176 aa, Homo sapiens
MMVNCHAHFWTTKAFLPTMLEINHGHIVTVASSLGLFSTAGVEDYCASKFGVVGFHESLSHELKAAEKDGIKTTLVCPYLVDTGMFRGCRIRKEIEPFLPPLKPDYCVKQAMKAILTDQPMICTPRLMYIVTFMKSILPFEAVVCMYRFLGADKCMYPFIAQRKQATNNNEAKNGI

SEQ ID NO: 259 - protein, 62 aa, Homo sapiens
MMVNCHAHFWTTKAFLPTMLEINHGHIVTVASSLGLFSTAGVEDYCASKFGVVGFHESLSHE

SEQ ID NO: 260 - protein, 382 aa, Homo sapiens
MRSHFLQWALATSRNKDQITNIFSGFAGLLAILLVVAVFCILWNWNKRKKRQVPYLRVTVMPLLTLPQTRQRAKNIYDILPWRQEDLGRHESRSMRIFSTESLLSRNSESPEHVPSQAGNAFQEHTAHIHATEYAVGIYDNAMVPQMCGNLTPSAHCINVRASRDCASISSEDSHDYVNVPTAEEIAETLASTKSPSRNLFVLPSTQKLEFTEERDEGCGDAGDCTSLYSPGAEDSDSLSNGEGSSQISNDYVNMTGLDLSAIQERQLWVAFQCCRDYENVPAADPSGSQQQAEKDVPSSNIGHVEDKTDDPGTHVQCVKRTFLASGDYADFQPFTQSEDSQMKHREEMSNEDSSDYENVLTAKLGGRDSEQGPGTQLLPDE

SEQ ID NO: 261 - protein, 398 aa, Homo sapiens
MDGVTPTLSTIRGRTLESSTLHVTPRSLDRNKDQITNIFSGFAGLLAILLVVAVFCILWNWNKRKKRQVPYLRVTVMPLLTLPQTRQRAKNIYDILPWRQEDLGRHESRSMRIFSTESLLSRNSESPEHVPSQAGNAFQEHTAHIHATEYAVGIYDNAMVPQMCGNLTPSAHCINVRASRDCASISSEDSHDYVNVPTAEEIAETLASTKSPSRNLFVLPSTQKLEFTEERDEGCGDAGDCTSLYSPGAEDSDSLSNGEGSSQISNDYVNMTGLDLSAIQERQLWVAFQCCRDYENVPAADPSGSQQQAEKDVPSSNIGHVEDKTDDPGTHVQCVKRTFLASGDYADFQPFTQSEDSQMKHREEMSNEDSSDYENVLTAKLGGRDSEQGPGTQLLPDE

SEQ ID NO: 262 - protein, 48 aa, Homo sapiens
MEAAETEAEAAALEVLAEVAGILEPVGLQEEAELPAKILVEFVVVCTR

SEQ ID NO: 263 - protein, 439 aa, Homo sapiens
MRRAALRLCALGKGQLTPGRGLTQGPQNPKKQGIFHIHEVRDKLREIVGASTNWRDHVKAMEERKLLHSFLAKSQDGLPPRRMKDSYIEVLLPLGSEPELREKYLTVQNTVRFGRILEDLDSLGVLICYMHNKIHSAKMSPLSIVTALVDKIDMCKKSLSPEQDIKFSGHVSWVGKTSMEVKMQMFQLHGDEFCPVLDATFVMVARDSENKGPAFVNPLIPESPEEEELFRQGELNKGRRIAFSSTSLLKMAPSAEERTTIHEMFLSTLDPKTISFRSRVLPSNAVWMENSKLKSLEICHPQERNIFNRIFGGFLMRKAYELAWATACSFGGSRPFVVAVDDIMFQKPVEVGSLLFLSSQVCFTQNNYIQVRVHSEVASLQEKQHTTTNVFHFTFMSEKEVPLVFPKTYGESMLYLDGQRHFNSMSGPATLRKDYLVEP

SEQ ID NO: 264 - protein, 448 aa, Homo sapiens
MRRAALRLCALGKGQLTPGRGLTQGPQNPKKQGIFHIHEACSSIHVNHVRDKLREIVGASTNWRDHVKAMEERKLLHSFLAKSQDGLPPRRMKDSYIEVLLPLGSEPELREKYLTVQNTVRFGRILEDLDSLGVLICYMHNKIHSAKMSPLSIVTALVDKIDMCKKSLSPEQDIKFSGHVSWVGKTSMEVKMQMFQLHGDEFCPVLDATFVMVARDSENKGPAFVNPLIPESPEEEELFRQGELNKGRRIAFSSTSLLKMAPSAEERTTIHEMFLSTLDPKTISFRSRVLPSNAVWMENSKLKSLEICHPQERNIFNRIFGGFLMRKAYELAWATACSFGGSRPFVVAVDDIMFQKPVEVGSLLFLSSQVCFTQNNYIQVRVHSEVASLQEKQHTTTNVFHFTFMSEKEVPLVFPKTYGESMLYLDGQRHFNSMSGPATLRKDYLVEP

SEQ ID NO: 265 - protein, 46 aa, Homo sapiens
MRRAALRLCALGKGQLTPGRGLTQGPQNPKKQGIFHIHEGKHGVSV

SEQ ID NO: 266 - protein, 663 aa, Homo sapiens
MSARLPVLSPPRWPRLLLLSLLLLGAVPGPRRSGAFYLPGLAPVNFCDEEKKSDECKAEIELFVNRLDSVESVLPYEYTAFDFCQASEGKRPSENLGQVLFGERIEPSPYKFTFNKKETCKLVCTKTYHTEKAEDKQKLEFLKKSMLLNYQHHWIVDNMPVTWCYDVEDGQRFCNPGFPIGCYITDKGHAKDACVISSDFHERDTFYIFNHVDIKIYYHVVETGSMGARLVAAKLEPKSFKHTHIDKPDCSGPPMDISNKASGEIKIAYTYSVSFEEDDKIRWASRWDYILESMPHTHIQWFSIMNSLVIVLFLSGMVAMIMLRTLHKDIARYNQMDSTEDAQEEFGWKLVHGDIFRPPRKGMLLSVFLGSGTQILIMTFVTLFFACLGFLSPANRGALMTCAVVLWVLLGTPAGYVAARFYKSFGGEKWKTNVLLTSFLCPGIVFADFFIMNLILWGEGSSAAIPFGTLVAILALWFCISVPLTFIGAYFGFKKNAIEHPVRTNQIPRQIPEQSFYTKPLPGIIMGGILPFGCIFIQLFFILNSIWSHQMYYMFGFLFLVFIILVITCSEATILLCYFHLCAEDYHWQWRSFLTSGFTAVYFLIYAVHYFFSKLQITGTASTILYFGYTMIMVLIFFLFTGTIGFFACFWFVTKIYSVVKVD

SEQ ID NO: 267 - protein, 390 aa, Homo sapiens
MGQRLSGGRSCLDVPGRLLPQPPPPPPPVRRKLALLFAMLCVWLYMFLYSCAGSCAAAPGLLLLGSGSRAAHDPPALATAPDGTPPRLPFRAPPATPLASGKEMAEGAASPEEQSPEVPDSPSPISSFFSGSGSKQLPQAIIIGVKKGGTRALLEFLRVHPDVRAVGAEPHFFDRSYDKGLAWYRDLMPRTLDGQITMEKTPSYFVTREAPARISAMSKDTKLIVVVRDPVTRAISDYTQTLSKRPDIPTFESLTFKNRTAGLIDTSWSAIQIGIYAKHLEHWLRHFPIRQMLFVSGERLISDPAGELGRVQDFLGLKRIITDKHFYFNKTKGFPCLKKAEGSSRPHCLGKTKGRTHPEIDREVVRRLREFYRPFNLKFYQMTGHDFGWD

SEQ ID NO: 268 - protein, 390 aa, Homo sapiens; identical to SEQ ID NO: 267

SEQ ID NO: 269 - protein, 649 aa, Homo sapiens
MSSFGAGKTKEVIFSVEDGSVKMFLRGRPVPMMIPDELAPTYSLDTRSELPSCRLKLEWVYGYRGRDCRANLYLLPTGEIVYFVASVAVLYSVEEQRQRHYLGHNDDIKCLAIHPDMVTIATGQVAGTTKEGKPLPPHVRIWDSVSLSTLHVLGLGVFDRAVCCVGFSKSNGGNLLCAVDESNDHMLSVWDWAKETKVVDVKCSNEAVLVATFHPTDPTVLITCGKSHIYFWTLEGGSLSKRQGLFEKHEKPKYVLCVTFLEGGDVVTGDSGGNLYVWGKGGNRITQAVLGAHDGGVFGLCALRDGTLVSGGGRDRRVVLWGSDYSKLQEVEVPEDFGPVRTVAEGHGDTLYVGTTRNSILQGSVHTGFSLLVQGHVEELWGLATHPSRAQFVTCGQDKLVHLWSSDSHQPLWSRIIEDPARSAGFHPSGSVLAVGTVTGRWLLLDTETHDLVAIHTDGNEQISVVSFSPDGAYLAVGSHDNLVYVYTVDQGGRKVSRLGKCSGHSSFITHLDWAQDSSCFVTNSGDYEILYWDPATCKQITSADAVRNMEWATATCVLGFGVFGIWSEGADGTDINAVARSHDGKLLASADDFGKVHLFSYPCCQPRALSHKYGGHSSHVTNVAFLWDDSMALTTGGKDTSVLQWRVV

SEQ ID NO: 270 - protein, 145 aa, Homo sapiens
MSSFGAGKTKEVIFSVAWPSTQIWSPSPRDRWREPLRKGSRCRPTCASGTQFPSPPYTCWAWGCLTEPCAVWASPNLMEATCCVQWMNPMITCSRCGTGPRRPRWWMSSAPMRLYWWPPSTPRTPLCLSPAGNLTSTSGPWRGAA

SEQ ID NO: 271 - protein, 76 aa, Homo sapiens
XGRKVSRLGKCSGHSSFITHLDWAQDSSCFVTNSGDYEILYLEMGFHHLGQAGLELLTSGDPARCCGSCCLLGLQT

SEQ ID NO: 272 - protein, 225 aa, Homo sapiens
MFLRGRPVPMMIPDELAPTYSLDTRSELPSCRLKLEWVYGYRGRDCRANLYLLPTGEIVYFVASVAVLYSVEEQRQRHYLGHNDDIKCLAIHPDMVTIATGQVAGTTKEGKPLPPHVRIWDSVSLSTLHVLGLGVFDRAVCCVGFSKSNGGNLLCAVDESNDHMLSVWDWAKETKVVDVKCSNEAVLVATFHPTDPTVLITCGKSHIYFWTLEGGSLSKRQGLFE

SEQ ID NO: 273 - protein, 850 aa, Homo sapiens
MLERRALLWQREAGPGWGDRARAGTGGAGGGCGGAMAERGPAFCGLYDTSSLLRYCNDDNLSGTSGMEVDDRVSALEQRLQLQEDELAVLKAALADALRRLRACEEQGAALRARGTPKGRAPPRLGTTASVCQLLKGLPTRTPLNGSGPPRRVGGYATSPSSPKKEATSGRSSVRRYLSPERLASVRREDPRSRTTSSSSNCSAKKEGKTKEVIFSVEDGSVKMFLRGRPVPMMIPDELAPTYSLDTRSELPSCRLKLEWVYGYRGRDCRANLYLLPTGEIVYFVASVAVLYSVEEQRQRHYLGHNDDIKCLAIHPDMVTIATGQVAGTTKEGKPLPPHVRIWDSVSLSTLHVLGLGVFDRAVCCVGFSKSNGGNLLCAVDESNDHMLSVWDWAKETKVVDVKCSNEAVLVATFHPTDPTVLITCGKSHIYFWTLEGGSLSKRQGLFEKHEKPKYVLCVTFLEGGDVVTGDSGGNLYVWGKGGNRITQAVLGAHDGGVFGLCALRDGTLVSGGGRDRRVVLWGSDYSKLQEVEVPEDFGPVRTVAEGHGDTLYVGTTRNSILQGSVHTGFSLLVQGHVEELWGLATHPSRAQFVTCGQDKLVHLWSSDSHQPLWSRIIEDPARSAGFHPSGSVLAVGTVTGRWLLLDTETHDLVAIHTDGNEQISVVSFSPDGAYLAVGSHDNLVYVYTVDQGGRKVSRLGKCSGHSSFITHLDWAQDSSCFVTNSGDYEILYWDPATCKQITSADAVRNMEWATATCVLGFGVFGIWSEGADGTDINAVARSHDGKLLASADDFGKVHLFSYPCCQPRALSHKYGGHSSHVTNVAFLWDDSMALTTGGKDTSVLQWRVV

SEQ ID NO: 274 - protein, 73 aa, Homo sapiens
XHQPLWSRIIEDPARSAGFHPSGSVLAVGTVTGRWLLLDTETHDLVAIHTDGNEQISVVSFSPGPFQFYHPPG

SEQ ID NO: 275 - protein, 121 aa, Homo sapiens
XLCAVDESNDHMLSVWDWAKETKVVDVKVPEDFGPVRTVAEGHGDTLYVGTTRNSILQGSVHTGFSLLVQGHVEELWGLATHPSRAQFVTCGQDKLVHLWSSDSHQPLWSRIIEDPARSAG

SEQ ID NO: 276 - protein, 427 aa, Homo sapiens
MSSFGAGKTKEVIFSVEDGSVKMFLRGRPVPMMIPDELAPTYSLDTRSELPSCRLKLEWVYGYRGRDCRANLYLLPTGEIVYFVASVAVLYSVEEQRQRHYLGHNDDIKCLAIHPDMVTIATGQVAGTTKEGKPLPPHVRIWDSVSLSTLHVLGLGVFDRAVCCVGFSKSNGGNLLCAVDESNDHMLSVWDWAKETKVVDVKCSNEAVLVATFHPTDPTVLITCGKSHIYFWTLEGGSLSKRQGLFEKHEKPKYVLCVTFLEGGDVVTGDSGGNLYVWGKGGNRITQAVLGAHDGGVFGLCALRDGTLVSGGGRDRRVVLWGSDYSKLQEVEVPEDFGPVRTVAEGHGDTLYVGTTRNSILQGSVHTGFSLLVQGHVEELWGLATHPSRAQFVTCGQDKLVHLWSSDSHQPLWSRIIEMAAAGHGDP

SEQ ID NO: 277 - protein, 773 aa, Homo sapiens
MSSFGAGKTKEVIFSVEDGSVKMFLRGRPVPMMIPDELAPTYSLDTRSELPSCRLKLEWVYGYRGRDCRANLYLLPTGEIVYFVASVAVLYSVEEQRQRHYLGHNDDIKCLAIHPDMVTIATGQVAGTTKEGKPLPPHVRIWDSVSLSTLHVLGLGVFDRAVCCVGFSKSNGGNLLCAVDESNDHMLSVWDWAKETKVVDVKCSNEAVLVATFHPTDPTVLITCGKSHIYFWTLEGGSLSKRQGLFEKHEKPKYVLCVTFLEGGDVVTGDSGGNLYVWGKGGNRITQAVLGAHDGGVFGLCALRDGTLVSGGGRDRRVVLWGSDYSKLQEVEVPEDFGPVRTVAEGHGDTLYVGTTRNSILQGSVHTGFSLLVQGHVEELWGLATHPSRAQFVTCGQDKLVHLWSSDSHQPLWSRIIEDPARSAGFHPSGSVLAVGTVTGRWLLLDTETHDLVAIHTDGNEQISVVSFSPDGAYLAVGSHDNLVYVYTVDQGGRKVSRLGKCSGHSSFITHLDWAQDSSCFVTNSGDYEILYWDPATCKQITSADAVRNMEWATATCVLGFGVFGIWSEGADGTDINAVARSHDGKLLASADDFGKVHLFSYPCCQPRVLGKWPSAAEAALSSGIRVLFRPLVASLGPAWKNVPRKFCWKSAGDSPRDPPPRPQGHSQPSFPSSGPWETAVCKFYRQGRWREVRVFASWAPPCIPHSQWLWFPSSEPQFLWELELSLETGNRLPPCPFCPRLAMASCPLRMVPWGRPILCGFLHSAHPSINCP

SEQ ID NO: 278 - protein, 98 aa, Homo sapiens
MFLRGRPVPMMIPDELAPTYSLDTRSELPSCRLKLEWVYGYRGRDCRANLYLLPTGEIVYFVASVAVLYSVEEQRQRHYLGHNDDIKCLAIHPDMVTI

SEQ ID NO: 279 - protein, 111 aa, Homo sapiens
MFLRGRPVPMMIPDELAPTYSLDTRSELPSCRLKLEWVYGYRGRDCRANLYLLPTGEIVYFVASVAVLYSVEEQRQRHYLGHNDDIKCLAIHPDMVTIATGQVAGTTKEGK

SEQ ID NO: 280 - protein, 51 aa, Homo sapiens
WTLEGGSLSKRQGLFEKHEKPKYVLCVTFLEGGDVVTGDSGGNLYVWGKGP

SEQ ID NO: 281 - protein, 148 aa, Homo sapiens
MFLRGRPVPMMIPDELAPTYSLDTRSELPSCRLKLEWVYGYRGRDCRANLYLLPTGEIVYFVASVAVLYSVEEQRQRHYLGHNDDIKCLAIHPDMVTIATGQVAGTTKEGKPLPPHVRIWDSVSLSTLHVLGLGVFDRAVCCVGFSKS

SEQ ID NO: 282 - protein, 91 aa, Homo sapiens
PLPPHVRIWDSVSLSTLHVLGLGVFDRAVCCVGFSKSNGGNLLCAVDESNDHMLSVWDWAKETKVVDVKAGVQWHNLGSLQPPPPRFQRFS

SEQ ID NO: 283 - protein, 133 aa, Homo sapiens
MFLRGRPVPMMIPDELAPTYSLDTRSELPSCRLKLEWVYGYRGRDCRANLYLLPTGEIVYFVASVAVLYSVEEQRQRHYLGHNDDIKCLAIHPDMVTIATGQVAGTTKEGKPLPPHVRIWDSVSLSTLHVLGL

SEQ ID NO: 284 - protein, 252 aa, Homo sapiens
MKMKLFQTICRQLRSSKFSVESAALVAFSTSSYSCGRKKKVNPYEEVDQEKYSNLVQSVLSSRGVAQTPGSVEEDALLCGPVSKHKLPNQGEDRRVPQNWFPIFNPERSDKPNASDPSVPLKIPLQRNVIPSVTRVLQQTMTKQQVFLLERWKQRMILELGEDGFKEYTSSSMWLNCGGLQRWITCPPTFHGCRALFPVLDQVASSTRRIYGKEKEPEYSETRIFRIGSKLLFGNIQHLLTVWEHILLFTPV

SEQ ID NO: 285 - protein, 264 aa, Homo sapiens
MKMKLFQTICRQLRSSKFSVESAALVAFSTSSYSCGRKKKVNPYEEVDQEKYSNLVQSVLSSRGVAQTPGSVEEDALLCGPVSKHKLPNQDVFLQGKRFHEALESILSPQETLKERDENLLKSGYIESVQHILKDVSGVRALESAVQHETLNYIGLLDCVAEYQGKLCVIDWKTSEKPKPFIQSTFDNPLQVVAYMGAMNHDTNYSFQVQCGLIVVAYKDGSPAHPHFMDAELCSQYWTKWLLRLEEYTEKKKNQNIQKPEYSE

SEQ ID NO: 286 - protein, 344 aa, Homo sapiens
MKMKLFQTICRQLRSSKFSVESAALVAFSTSSYSCGRKKKVNPYEEVDQEKYSNLVQSVLSSRGVAQTPGSVEEDALLCGPVSKHKLPNQGEDRRVPQNWFPIFNPERSDKPNASDPSVPLKIPLQRNVIPSVTRVLQQTMTKQQVFLLERWKQRMILELGEDGFKEYTSNVFLQGKRFHEALESILSPQETLKERDENLLKSGYIESVQHILKDVSGVRALESAVQHETLNYIGLLDCVAEYQGKLCVIDWKTSEKPKPFIQSTFDNPLQVVAYMGAMNHDTNYSFQVQCGLIVVAYKDGSPAHPHFMDAELCSQYWTKWLLRLEEYTEKKKNQNIQKPEYSE

SEQ ID NO: 287 - protein, 355 aa, Homo sapiens
MGPGRCLLTALLLLALAPPPEASQYCGRLEYWNPDNKCCSSCLQRFGPPPCPDYEFRENCGLNDHGDFVTPPFRKCSSGQCNPDGAELCSPCGGGAVTPTPAAGGGRTPWRCRERPVPAKGHCPLTPGNPGAPSSQERSSPASSIAWRTPEPVPQQAWPNFLPLVVLVLLLTLAVIAILLFILLWHLCWPKEKADPYPYPGLVCGVPNTHTPSSSHLSSPGALETGDTWKEASLLPLLSRELSSLASQPLSRLLDELEVLEELIVLLDPEPGPGGGMAHGTTRHLAARYGLPAAWSTFAYSLRPSRSPLRALIEMVVAREPSASLGQLGTHLAQLGRADALRVLSKLGSSGVCWA

SEQ ID NO: 288 - protein, 132 aa, Homo sapiens
MGPGRCLLTALLLLALAPPPEASQYCGRLEYWNPDNKCCSSCLQRFGPPPCPDYEFRENCGLNDHGDFVTPPFRKCSSGQCNPDGAELCSPCGGGAVTPTPAAGGGRTPWRCREAGPCQGALPPHTWKPRRP

SEQ ID NO: 289 - protein, 187 aa, Homo sapiens
MGPGRCLLTALLLLALAPPPEASQYCGRLEYWNPDNKCCSSCLQRFGPPPCPGALETGDTWKEASLLPLLSRELSSLASQPLSRLLDELEVLEELIVLLDPEPGPGGGMAHGTTRHLAARYGLPAAWSTFAYSLRPSRSPLRALIEMVVAREPSASLGQLGTHLAQLGRADALRVLSKLGSSGVCWA

SEQ ID NO: 290 - protein, 138 aa, Homo sapiens
MGPGRCLLTALLLLALAPPPEASQYCGRLEYWNPDNKCCSSCLQRFGPPPCPDYEFRENCGLNDHGDFVTPPFRKCSSGQCNPDGAELCSPCGGGAVTPTPAAGGGRTPWRCRERPVPAKGHCPLTPGNPGAPSSQER

SEQ ID NO: 291 - protein, 74 aa, Homo sapiens
XNCGLNDHGDFVTPPFRKCSSGQCNPDGAELCSPCGGGAVTPTPAAGGGRTPWRCREAPWRQGTHGRRPHYFHS

SEQ ID NO: 292 - protein, 355 aa, Homo sapiens; identical to SEQ ID NO: 287

SEQ ID NO: 293 - protein, 60 aa, Homo sapiens
XGRLEYWNPDNKCCSSCLQRFGPPPCPGENPRPSLAWAELAEAGPCQGALPPHTWKPRRP

SEQ ID NO: 294 - protein, 167 aa, Homo sapiens
MGPGRCLLTALLLLALAPPPEASQYCGRLEYWNPDNKCCSSCLQRFGPPPCPELSSLASQPLSRLLDELEVLEELIVLLDPEPGPGGGMAHGTTRHLAARYGLPAAWSTFAYSLRPSRSPLRALIEMVVAREPSASLGQLGTHLAQLGRADALRVLSKLGSSGVCWA

SEQ ID NO: 295 - protein, 1742 aa, Homo sapiens
MAVAELYTQYNRVWIPDPEEVWKSAEIAKDYRVGDKVLRLLLEDGTELDYSVNPESLPPLRNPDILVGENDLTALSYLHEPAVLHNLRIRFAESKLIYTYSGIILVAMNPYKQLPIYGDAIIHAYSGQNMGDMDPHIFAVAEEAYKQMARNNRNQSIIVSGESGAGKTVSARYAMRYFATVSKSGSNAHVEDKVLASNPITEAVGNAKTTRNDNSSRFGKYTEISFDEQNQIIGANMSTYLLEKSRVVFQSENERNYHIFYQLCASAQQSEFKHLKLGSAEEFNYTRMGGNTVIEGVNDRAEMVETQKTFTLLGFKEDFQMDVFKILAAILHLGNVQITAVGNERSSVSEDDSHLKVFCELLGLESGRVAQWLCNRKIVTSSETVVKPMTRPQAVNARDALAKKIYAHLFDFIVERINQALQFSGKQHTFIGVLDIYGFETFDVNSFEQFCINYANEKLQQQFNMHVFKLEQEEYMKEDIPWTLIDFYDNQPVIDLIEAKMGILELLDEECLLPHGTDENWLQKLYNNFVNRNPLFEKPRMSNTSFVIQHFADKVEYKCEGFLEKNRDTVYDMLVEILRASKFHLCANFFQENPTPPSPFGSMITVKSAKQVIKPNSKHFRTTVGSKFRSSLYLLMETLNATTPHYVRCIKPNDEKLPFEFDSKRIVQQLRACGVLETIRISAQSYPSRWTYIEFYSRYGILMTKQELSFSDKKEVCKVVLHRLIQDSNQYQFGKTKIFFRAGQVAYLEKLRLDKLRQSCVMVQKHMRGWLQRKKFLRERRAALIIQQYFRGQQTVRKAITAVALKEAWAAIIIQKHCRGYLVRSLYQLIRMATITMQAYSRGFLARRRYRKMLEEHKAVILQKYARAWLARRRFQSIRRFVLNIQLTYRVQRLQKKLEDQNKENHGLVEKLTSLAALRAGDVEKIQKLEAELEKAATHRRNYEEKGKRYRDAVEEKLAKLQKHNSELETQKEQIQLKLQEKTEELKEKMDNLTKQLFDDVQKEERQRMLLEKSFELKTQDYEKQIQSLKEEIKALKDEKMQLQHLVEGEHVTSDGLKAEVARLSKQVKTISEFEKEIELLQAQKIDVEKHVQSQKREMREKMSEITKQLLESYDIEDVRSRLSVEDLEHLNEDGELWFAYEGLKKATRVLESHFQSQKDCYEKEIEALNFKVVHLSQEINHLQKLFREENDINESIRHEVTRLTSENMMIPDFKQQISELEKQKQDLEIRLNEQAEKMKGKLEELSNQLHRSQEEEGTQRKALEAQNEIHTKEKEKLIDKIQEMQEASDHLKKQFETESEVKCNFRQEASRLTLENRDLEEELDMKDRVIKKLQDQVKTLSKTIGKANDVHSSSGPKEYLGMLQYKREDEAKLIQNLILDLKPRGVVVNMIPGLPAHILFMCVRYADSLNDANMLKSLMNSTINGIKQVVKEHLEDFEMLSFWLSNTCHFLNCLKQYSGEEEFMKHNSPQQNKNCLNNFDLSEYRQILSDVAIRIYHQFIIIMEKNIQPIIVPGMLEYESLQGISGLKPTGFRKRSSSIDDTDGYTMTSVLQQLSYFYTTMCQNGLDPELVRQAVKQLFFLIGAVTLNSLFLRKDMCSCRKGMQIRCNISYLEEWLKDKNLQNSLAKETLEPLSQAAWLLQVKKTTDSDAKEIYERCTSLSAVQIIKILNSYTPIDDFEKRVTPSFVRKVQALLNSREDSSQLMLDTKYLFQVTFPFTPSPHALEMIQIPSSFKLGFLNRL

SEQ ID NO: 296 - protein, 612 aa, Homo sapiens
MAAAGRLPSSWALFSPLLAGLALLGVGPVPARALHNVTAELFGAEAWGTLAAFGDLNSDKQTDLFVLRERNDLIVFLADQNAPYFKPKVKVSFKNHSALITSVVPGDYDGDSQMDVLLTYLPKNYAKSELGAVIFWGQNQTLDPNNMTILNRTFQDEPLIMDFNGDLIPDIFGITNESNQPQILLGGNLSWHPALTTTSKMRIPHSHAFIDLTEDFTADLFLTTLNATTSTFQFEIWENLDGNFSVSTILEKPQNMMVVGQSAFADFDGDGHMDHLLPGCEDKNCQKSTIYLVRSGMKQWVPVLQDFSNKGTLWGFVPFVDEQQPTEIPIPITLHIGDYNMDGYPDALVILKNTSGSNQQAFLLENVPCNNASCEEARRMFKVYWELTDLNQIKDAMVATFFDIYEDGILDIVVLSKGYTKNDFAIHTLKNNFEADAYFVKVIVLSGLCSNDCPRKITPFGVNQPGPYIMYTTVDANGYLKNGSAGQLSQSAHLALQLPYNVLGLGRSANFLDHLYVGIPRPSGEKSIRKQEWTAIIPNSQLIVIPYPHNVPRSWSAKLYLTPSNIVLLTAIALIGVCVFILAIIGILHWQEKKADDREKRQEAHRFHFDAM

SEQ ID NO: 297 - protein, 464 aa, Homo sapiens
MDVLLTYLPKNYAKSELGAVIFWGQNQTLDPNNMTILNRTFQDEPLIMDFNGDLIPDIFGITNESNQPQILLGGNLSWHPALTTTSKMRIPHSHAFIDLTEDFTADLFLTTLNATTSTFQFEIWENLDGNFSVSTILEKPQNMMVVGQSAFADFDGDGHMDHLLPGCEDKNCQKSTIYLVRSGMKQWVPVLQDFSNKGTLWGFVPFVDEQQPTEIPIPITLHIGDYNMDGYPDALVILKNTSGSNQQAFLLENVPCNNASCEEARRMFKVYWELTDLNQIKDAMVATFFDIYEDGILDIVVLSKGYTKNDFAIHTLKNNFEADAYFVKVIVLSGLCSNDCPRKITPFGVNQPGPYIMYTTVDANGYLKNGSAGQLSQSAHLALQLPYNVLGLGRSANFLDHLYVGIPRPSGEKSIRKQEWTAIIPNSQLIVIPYPHNVPRSSHRCLCFHLGNNWHFTLAGKESR

SEQ ID NO: 298 - protein, 74 aa, Homo sapiens
MDVLLTYLPKNYAKSELGAVIFWGQNQTLDPNNMTILNRTFQDEPLIMDFNGDLIPDIFGITNESNQPQILLGG

SEQ ID NO: 299 - protein, 178 aa, Homo sapiens
VLSKGYTKNDFAIHTLKNNFEADAYFVKVIVLSGLCSNDCPRKITPFGVNQPGPYIMYTTVDANGYLKNGSGRSANFLDHLYVGIPRPSGEKSIRKQEWTAIIPNSQLIVIPYPHNVPRSWSAKLYLTPSNIVLLTAIALIGVCVFILAIIGILHWQEKKADDREKRQEAHRFHFDAM

SEQ ID NO: 300 - protein, 113 aa, Homo sapiens
XKPKVKVSFKNHSALITSVVPGDYDGDSQMDVLLTYLPKNYAKSELGAVIFWGQNQTLDPNNMTILNRTFQDEPLIMDFNGDLIPDIFGITNESNQPQILLGGHTTLNAPHLF

SEQ ID NO: 301 - protein, 431 aa, Homo sapiens
MAEITNIRPSFDVSPVVAGLIGASVLVVCVSVTVFVWSCCHQQAEKKQKNPPYKFIHMLKGISIYPETLSNKKKIIKVRRDKDGPGREGGRRNLLVDAAEAGLLSRDKDPRGPSSGSCIDQLPIKMDYGEELRSPITSLTPGESKTTSPSSPEEDVMLGSLTFSVDYNFPKKALVVTIQEAHGLPVMDDQTQGSDPYIKMTILPDKRHRVKTRVLRKTLDPVFDETFTFYGIPYSQLQDLVLHFLVLSFDRFSRDDVIGEVMVPLAGVDPSTGKVQLTRDIIKRNIQKCISRGELQVSLSYQPVAQRMTVVVLKARHLPKMDITGLSGNPYVKVNVYYGRKRIAKKKTHVKKCTLNPIFNESFIYDIPTDLLPDISIEFLVIDFDRTTKNEVVGRLILGAHSVTASGAEHWREVCESPRKPVAKWHSLSEY

SEQ ID NO: 302 - protein, 513 aa, Homo sapiens
MSSKPEPKDVHQLNGTGPSASPCSSDGPGREPLAGTSEFLGPDGAGVEVVIESRANAKGVREEDALLENGSQSNESDDVSTDRGPAPPSPLKETSFSIGLQVLFPFLLAGFGTVAAGMVLDIVQHWEVFQKVTEVFILVPALLGLKGNLEMTLASRLSTAANIGHMDTPKELWRMITGNMALIQVQATVVGFLASIAAVVFGWIPDGHFSIPHAFLLCASSVATAFIASLVLGMIMIGVIIGSRKIGINPDNVATPIAASLGDLITLALLSGISWGLYLELNHWRYIYPLVCAFFVALLPVWVVLARRSPATREVLYSGWEPVIIAMAISSVGGLILDKTVSDPNFAGMAVFTPVINGVGGNLVAVQASRISTFLHMNGMPGENSEQAPRRCPSPCTTFFSPDVNSRSARVLFLLVVPGHLVFLYTISCMQGGHTTLTLIFIIFYMTAALLQVLILLYIADWMVHWMWGRGLDPDNFSIPYLTALGDLLGTGLLALSFHVLWLIGDRDTDVGD

SEQ ID NO: 303 - protein, 1226 aa, Homo sapiens
MDREERKTINQGQEDEMEIYGYNLSRWKLAIVSLGVICSGGFLLLLLYWMPEWRVKATCVRAAIKDCEVVLLRTTDEFKMWFCAKIRVLSLETYPVSSPKSMSNKLSNGHAVCLIENPTEENRHRISKYSQTESQQIRYFTHHSVKYFWNDTIHNFDFLKGLDEGVSCTSIYEKHSAGLTKGMHAYRKLLYGVNEIAVKVPSVFKLLIKEVLNPFYIFQLFSVILWSTDEYYYYALAIVVMSIVSIVSSLYSIRKQYVMLHDMVATHSTVRVSVCRVNEEIEEIFSTDLVPGDVMVIPLNGTIMPCDAVLINGTCIVNESMLTGESVPVTKTNLPNPSVDVKGIGDELYNPETHKRHTLFCGTTVIQTRFYTGELVKAIVVRTGFSTSKGQLVRSILYPKPTDFKLYRDAYLFLLCLVAVAGIGFIYTIINSILNEVQVGVIIIESLDIITITVPPALPAAMTAGIVYAQRRLKKIGIFCISPQRINICGQLNLVCFDKTGTLTEDGLDLWGIQRVENARFLSPEENVCNEMLVKSQFVACMATCHSLTKIEGVLSGDPLDLKMFEAIGWILEEATEEETALHNRIMPTVVRPPKQLLPESTPAGNQEMELFELPATYEIGIVRQFPFSSALQRMSVVARVLGDRKMDAYMKGAPEAIAGLCKPETVPVDFQNVLEDFTKQGFRVIALAHRKLESKLTWHKVQNISRDAIENNMDFMGLIIMQNKLKQETPAVLEDLHKANIRTVMVTGDSMLTAVSVARDCGMILPQDKVIIAEALPPKDGKVAKINWHYADSLTQCSHPSAIDPEAIPVKLVHDSLEDLQMTRYHFAMNGKSFSVILEHFQDLVPKLMLHGTVFARMAPDQKTQLIEALQNVDYFVGMCGDGANDCGALKRAHGGISLSELEASVASPFTSKTPSISCVPNLIREGRAALITSFCVFKFMALYSIIQYFSVTLLYSILSNLGDFQFLFIDLAIILVVVFTMSLNPAWKELVAQRPPSGLISGALLFSVLSQIIICIGFQSLGFFWVKQQPWYEVWHPKSDACNTTGSGFWNSSHVDNETELDEHNIQNYENTTVFFISSFQYLIVAIAFSKGKPFRQPCYKNYFFVFSVIFLYIFILFIMLYPVASVDQVLQIVCVPYQWRVTMLIIVLVNAFVSITVEESVDRWGKCCLPWALGCRKKTPKAKYMYLAQELLVDPEWPPKPQTTTEAKALVKENGSCQIITIT

SEQ ID NO: 304 - protein, 162 aa, Homo sapiens
XPFRQPCYKNYFFVFSVIFLYIFILFIMLYPVASVDQVLQIVCVPYQWRVTMLIIVLVNAFVSITVENFFLDMVLWKVVFNRDKQGEYRFSTTQPPQESVDRWGKCCLPWALGCRKKTPKAKYMYLAQELLVDPEWPPKPQTTTEAKALVKENGSCQIITIT

SEQ ID NO: 305 - protein, 1226 aa, Homo sapiens; identical to SEQ ID NO: 303

SEQ ID NO: 306 - protein, 47 aa, Homo sapiens
MDREERKTINQGQEDEMEIYGYNLSRWKLAIVSLGVICSGGFLLLLL

SEQ ID NO: 307 - protein, 103 aa, Homo sapiens
MDREERKTINQGQEDEMEIYGYNLSRWKLAIVSLGVICSGGFLLLLLYWMPEWRVKATCVRAAIKDCEVVLLRTTDEFKMWFCAKIRVLSLETYPVSSPKSMS

SEQ ID NO: 308 - protein, 701 aa, Homo sapiens
ISEIEEIFSTDLVPGDVMVIPLNGTIMPCDAVLINGTCIVNESMLTGESVPVTKTNLPNPSVDVKGIGDELYNPETHKRHTLFCGTTVIQTRFYTGELVKAIVVRTGFSTSKGQLVRSILYPKPTDFKLYRDAYLFLLCLVAVAGIGFIYTIINSILNEVQVGVIIIESLDIITITVPPALPAAMTAGIVYAQRRLKKIGIFCISPQRINICGQLNLVCFDKTGTLTEDGLDLWGIQRVENARFLSPEENVCNEMLVKSQFVACMATCHSLTKIEGVLSGDPLDLKMFEAIGWILEEATEEETALHNRIMPTVVRPPKQLLPESTPAGNQEMELFELPATYEIGIVRQFPFSSALQRMSVVARVLGDRKMDAYMKGAPEAIAGLCKPETVPVDFQNVLEDFTKQGFRVIALAHRKLESKLTWHKVQNISRDAIENNMDFMGLIIMQNKLKQETPAVLEDLHKANIRTVMVTGDSMLTAVSVARDCGMILPQDKVIIAEALPPKDGKVAKINWHYADSLTQCSHPSAIDPEAIPVKLVHDSLEDLQMTRYHFAMNGKSFSVILEHFQDLVPKLMLHGTVFARMAPDQKTQLIEALQNVDYFVGMCGDGANDCGALKRAHGGISLSELEASVASPFTSKTPSISCVPNLIREGRAALITSFCELALFSIVTYSLDHFIISILISNMLVLFFSDFHNCAFYSLV

SEQ ID NO: 309 - protein, 976 aa, Homo sapiens
MGENEDEKQAQAGQVFENFVQASTCKGTLQAFNILTRHLDLDPLDHRNFYSKLKSKVTTWKAKALWYKLDKRGSHKEYKRGKSCTNTKCLIVGGGPCGLRTAIELAYLGAKVVVVEKRDSFSRNNVLHLWPFTIHDLRGLGAKKFYGKFCAGSIDHISIRQLQLILFKVALMLGVEIHVNVEFVKVLEPPEDQENQKIGWRAEFLPTDHSLSEFEFDVIIGADGRRNTLEGFRRKEFRGKLAIAITANFINRNSTAEAKVEEISGVAFIFNQKFFQDLKEETGIDLENIVYYKDCTHYFVMTAKKQSLLDKGVIINDYIDTEMLLCAENVNQDNLLSYAREAADFATNYQLPSLDFAMNHYGQPDVAMFDFTCMYASENAALVRERQAHQLLVALVGDSLLEPFWPMGTGCARGFLAAFDTAWMVKSWNQGTPPLELLAERESLYRLLPQTTPENINKNFEQYTLDPGTRYPNLNSHCVRPHQVKHLYITKELEHYPLERLGSVRRSVNLSRKESDIRPSKLLTWCQQQTEGYQHVNVTDLTTSWRSGLALCAIIHRFRPELINFDSLNEDDAVENNQLAFDVAEREFGIPPVTTGKEMASAQEPDKLSMVMYLSKFYELFRGTPLRPVDSWRKNYGENADLSLAKSSISNNYLNLTFPRKRTPRVDGQTGENDMNKRRRKGFTNLDEPSNFSSRSLGSNQECGSSKEGGNQNKVKSMANQLLAKFEESTRNPSLMKQEKKSPSGFHFHPSHLRTVHPQESMRKSFPLNLGGSDTCYFCKKRVYVMERLSAEGHFFHRECFRCSICATTLRLAAYTFDCDEGKFYCKPHFIHCKTNSKQRKRRAELKQQREEEATWQEQEAPRRDTPTESSCAVAAIGTLEGSPPGISTSFFRKVLGWPLRLPRDLCNWMQGLLQAAGLHIRDNAYNYCYMYELLSLGLPLLWAFSEVLAAMYRESEGSLESICNWVLRCFPVKLR

SEQ ID NO: 310 - protein, 633 aa, Homo sapiens
MAAAAAAATTAACSSGSAGTDAAGASGLQQPPPQPQPQPAAAAPAQPPPEPPRKPRMDPRRRQAALSFLTNISLDGRLPPQDAEWGGGEEGGAAKPGAGGACGARTRFSLLAAAERGGCIALAAPGTPAAGLAAGSGPCLPQPSSLPPLIPGGHATVSGPGVARGFASPLGAGRASGEQWQPPRPAPLAACAQLQLLDGSGAAGQEELEEDDAFISVQVPAAAFLGSGTPGSGSGSRGRLNSFTQGILPIAFSRPTSQNYCSLEQPGQGGSTSAFEQLQRSRRRLISQRSSLETLEDIEENAPLRRCRTLSGSPRPKNFKKIHFIKNMRQHDTRNGRIVLISGRRSFCSIFSVLPYRDSTQVGDLKLDGGRQSTGAVSLKEIIGLEGVELGADGKTVSYTQFLLPTNAFGARRNTIDSTSSFSQFRNLSHRSLSIGRASGTQGSLDTGSDLGDFMDYDPNLLDDPQWPCGKHKRVLIFPSYMTTVIDYVKPSDLKKDMNETFKEKFPHIKLTLSKIRSLKREMRKLAQEDCGLEEPTVAMAFVYFEKLALKGKLNKQNRKLCAGACVLLAAKIGSDLKKHEVKHLIDKLEEKFRLNRRELIAFEFPVLVALEFALHLPEHEVMPHYRRLVQSS

SEQ ID NO: 311 - protein, 38 aa, Homo sapiens
MRQHDTRNGRIVLISGRRSFCSIFSVLPYRDSTQVGQQ

SEQ ID NO: 312 - protein, 301 aa, Homo sapiens
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP

SEQ ID NO: 313 - protein, 273 aa, Homo sapiens
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAETQTLGSLPDINLTQISTLANELRDSRLANDLRDSGATIRIGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP

SEQ ID NO: 314 - protein, 116 aa, Homo sapiens
MQLSFWSHCRCFCMFSIPLLMCRVRDQICDCPECTLQENPFFSQPGAPILQCMGCCFSRAYPTPLRSKKTMLVQKNVTSESTCCVAKSYNRVTVMGGFKVENHTACHCSTCYYHKS

SEQ ID NO: 315 - protein, 147 aa, Homo sapiens
MDYYRKYAAIFLVTLSVFLHVLHSAPDVQETGFHHVAQAALKLLSSSNPPTKASQSARITDCPECTLQENPFFSQPGAPILQCMGCCFSRAYPTPLRSKKTMLVQKNVTSESTCCVAKSYNRVTVMGGFKVENHTACHCSTCYYHKS

SEQ ID NO: 316 - protein, 62 aa, Homo sapiens
MDYYRKYAAIFLVTLSVFLHVLHSAPDVQDCPECTLQENPFFSQPGAPILQCMGCCFSRAYP

SEQ ID NO: 317 - protein, 116 aa, Homo sapiens
MDYYRKYAAIFLVTLSVFLHVLHSAPDVQDCPECTLQENPFFSQPGAPILQCMGCCFSRAYPTPLRSKKTMLVQKNVTSESTCCVAKSYNRVTVMGGFKVENHTACHCSTCYYHKS

SEQ ID NO: 318 - protein, 101 aa, Homo sapiens
MDYYRKYAAIFLVTLSVFLHVLHSAPDVQDCPECTLQENPFFSQPGAPILQCMGCCFSRAYPTPLRSKKTMLVQKNVTSESTCCVAKSYNRVRTSRSPEAF

SEQ ID NO: 319 - protein, 316 aa, Homo sapiens
MTQGKLSVANKAPGTEGQQQVHGEKKEAPAVPSAPPSYEEATSGEGMKAGAFPPAPTAVPLHPSWAYVDPSSSSSYDNGFPTGDHELFTTFSWDDQKVRRVFVRKVYTILLIQLLVTLAVVALFTFCDPVKDYVQANPGWYWASYAVFFATYLTLACCSGPRRHFPWNLILLTVFTLSMAYLTGMLSSYYNTTSVLLCLGITALVCLSVTVFSFQTKFDFTSCQGVLFVLLMTLFFSGLILAILLPFQYVPWLHAVYAALGAGVFTLFLALDTQLLMGNRRHSLSPEEYIFGALNIYLDIIYIFTFFLQLFGTNRE

SEQ ID NO: 320 - protein, 111 aa, Homo sapiens
MKAGAFPPAPTAVPLHPSWAYVDPSSSSSYDNGFPTGDHELFTTFSWDDQKVRRVFVRKVYTILLIQLLVTLAVVALFTFCDPVKDYVQANPGWYWASYAVFFATYLTLAC

SEQ ID NO: 321 - protein, 208 aa, Homo sapiens
MTQGKLSVANKAPGTEGQQQVHGEKKEAPAVPSAPPSYEEATSGEGMKAGAFPPAPTAVPLHPSWAYVDPSSSSSYDNGFPTGDHELFTTFSWDDQKVRRVFVRKNPSHPQVYTILLIQLLVTLAVVALFTFCDPVKDYVQANPGWYWASYAVFFATYLTLACCSGPRRHFPWNLILLTVFTLSMAYLTGMLSSSTSPPARACSSCFS

SEQ ID NO: 322 - protein, 248 aa, Homo sapiens
MTQGKLSVANKAPGTEGQQQVHGEKKEAPAVPSAPPSYEEATSGEGMKAGAFPPAPTAVPLHPSWAYVDPSSSSSYDNGFPTGDHELFTTFSWDDQKVRRVFVRKVYTILLIQLLVTLAVVALFTFCDPVKDYVQANPGWYWASYAVFFATYLTLACCSGPRRHFPWNLILLTVFTLSMAYLTGMLSSYYNTTSVLLCLGITALVCLSVTVFSFQTKFDFTSCQGVLFVLLMTLFFSGLILAILLPFQ

SEQ ID NO: 323 - protein, 270 aa, Homo sapiens
MKAGAFPPAPTAVPLHPSWAYVDPSSSSSYDNGFPTGDHELFTTFSWDDQKVRRVFVRKVYTILLIQLLVTLAVVALFTFCDPVKDYVQANPGWYWASYAVFFATYLTLACCSGPRRHFPWNLILLTVFTLSMAYLTGMLSSYYNTTSVLLCLGITALVCLSVTVFSFQTKFDFTSCQGVLFVLLMTLFFSGLILAILLPFQYVPWLHAVYAALGAGVFTLFLALDTQLLMGNRRHSLSPEEYIFGALNIYLDIIYIFTFFLQLFGTNRE

SEQ ID NO: 324 - protein, 266 aa, Homo sapiens
MVYTSHYKSPGRRPLKTHPTVTYWRVNEGSSSSYDNGFPTGDHELFTTFSWDDQKVRRVFVRKVYTILLIQLLVTLAVVALFTFCDPVKDYVQANPGWYWASYAVFFATYLTLACCSGPRRHFPWNLILLTVFTLSMAYLTGMLSSYYNTTSVLLCLGITALVCLSVTVFSFQTKFDFTSCQGVLFVLLMTLFFSGLILAILLPFQYVPWLHAVYAALGAGVFTLFLALDTQLLMGNRRHSLSPEEYIFGALNIYLDIIYIFTFFL

SEQ ID NO: 325 - protein, 145 aa, Homo sapiens
XYVQANPGWYWASYAVFFATYLTLACCSGPRRHFPWNLILLTVFTLSMAYLTGMLSSYYNTTSVLLCLGITALVCLSVTVFSFQTKFDFTSCQGVLFVLLMTLFFSGLILAILLPFQYFLALDTQLLMGNRRHSLSPEEYIFGAL

SEQ ID NO: 326 - protein, 695 aa, Homo sapiens
XEGGQGPRGLGTVPWLRDLPGSENHMPWEEPAGEKPSCSHSQKAFHMEPAQKPCFTTDMVTWALLCISAETVRGEAPSQPRGIPHRSPVSVDDLWLEKTQRKKLQKQAHVERRLHIGAVHKDGVKCWRKTIITSPESLNLPRRSHPLSQSAPTGLNHMGWPEHTPGTAMPDGALDTAVCADEVGSEEDLYDDLHSSSHHYSHPGGGGEQLAINELISDGSVVCAEALWDHVTMDDQELGFKAGDVIEVMDATNREWWWGRVADGEGWFPASFVRLRVNQDEPADDDAPLAGNSGAEDGGAEAQSSKDQMRTNVINEILSTERDYIKHLRDICEGYVRQCRKRADMFSEEQLRTIFGNIEDIYRCQKAFVKALEQRFNRERPHLSELGACFLEHQADFQIYSEYCNNHPNACVELSRLTKLSKYVYFFEACRLLQKMIDISLDGFLLTPVQKICKYPLQLAELLKYTHPQHRDFKDVEAALHAMKNVAQLINERKRRLENIDKIAQWQSSIEDWEGEDLLVRSSELIYSGELTRVTQPQAKSQQRMFFLFDHQLIYCKKDLLRRDVLYYKGRLDMDGLEVVDLEDGKDRDLHVSIKNAFRLHRGATGDSHLLCTRKPEQKQRWLKAFAREREQVQLDQETGFSITELQRKQAMLNASKQQVTGKPKGRRTAAPPPRLPGPYPADIIPFSEPQSQAS

SEQ ID NO: 327 - protein, 573 aa, Homo sapiens
MTNSKGRSITDKTSGGPSSGGGFVDWTLRLNTIQSDKFLNLLLSMVPVIYQKNQEDRHKKANGIWQDGLSTAVQTFSNRSEQHMEYHSFSEQSFHANNGHASSSCSQKYDDYANYNYCDGRETSETTAMLQDEDISSDGDEDAIVEVTPKLPKESSGIMALQILVPFLLAGFGTVSAGMVLDIVQHWEVFRKVTEVFILVPALLGLKGNLEMTLASRLSTAVNIGKMDSPIEKWNLIIGNLALKQVQATVVGFLAAVAAIILGWIPEGKYYLDHSILLCSSSVATAFIASLLQGIIMVGVIVGSKKTGINPDNVATPIAASFGDLITLAILAWISQGLYSCLETYYYISPLVGVFFLALTPIWIIIAAKHPATRTVLHSGWEPVITAMVISSIGGLILDTTVSDPNLVGIVVYTPVINGIGGNLVAIQASRISTYLHLHSIPGELPDEPKGCYYPFRTFFGPGVNNKSAQVLLLLVIPGHLIFLYTIHLMKSGHTSLTIIFIVVYLFGAVLQVFTLLWIADWMVHHFWRKGKDPDSFSIPYLTALGDLLGTALLALSFHFLWLIGDRDGDVGD

SEQ ID NO: 328 - protein, 149 aa, Homo sapiens
XDDYANYNYCDGRETSETTAMLQDEDISSDGDEDAIVEVTPKLPKESSGIMALQILVPFLLAGFGTVSAGMVLDIVQVNIGKMDSPIEKWNLIIGNLALKQVQATVVGFLAAVAAIILGWIPEGKYYLDHSILLCSSSVATAFIASLLQ

SEQ ID NO: 329 - protein, 68 aa, Homo sapiens
XLKALKGYIKHEARKGNENQDSSTSKRKTLCGFYSHQPTTLARPVLWPCKSEYLGSEGVTQALCYLCS

SEQ ID NO: 330 - protein, 76 aa, Homo sapiens
MIIPSLEELDSLKYSDLQNLAKSLGLRANLRATKLLKALKGYIKHEARKGNENQFHSCCPGWSAMTRSRLTATSTS

SEQ ID NO: 331 - protein, 748 aa, Homo sapiens
MVLLRVLILLLSWAAGMGGQYGNPLNKYIRHYEGLSYNVDSLHQKHQRAKRAVSHEDQFLRLDFHAHGRHFNLRMKRDTSLFSDEFKVETSNKVLDYDTSHIYTGHIYGEEGSFSHGSVIDGRFEGFIQTRGGTFYVEPAERYIKDRTLPFHSVIYHEDDINYPHKYGPQGGCADHSVFERMRKYQMTGVEEVTQIPQEEHAANGPELLRKKRTTSAEKNTCQLYIQTDHLFFKYYGTREAVIAQISSHVKAIDTIYQTTDFSGIRNISFMVKRIRINTTADEKDPTNPFRFPNIGVEKFLELNSEQNHDDYCLAYVFTDRDFDDGVLGLAWVGAPSGSSGGICEKSKLYSDGKKKSLNTGIITVQNYGSHVPPKVSHITFAHEVGHNFGSPHDSGTECTPGESKNLGQKENGNYIMYARATSGDKLNNNKFSLCSIRNISQVLEKKRNNCFVESGQPICGNGMVEQGEECDCGYSDQCKDECCFDANQPEGRKCKLKPGKQCSPSQGPCCTAQCAFKSKSEKCRDDSDCAREGICNGFTALCPASDPKPNFTDCNRHTQVCINGQCAGSICEKYGLEECTCASSDGKDDKELCHVCCMKKMDPSTCASTGSVQWSRHFSGRTITLQPGSPCNDFRGYCDVFMRCRLVDADGPLARLKKAIFSPELYENIAEWIVAHWWAVLLMGIALIMLMAGFIKICSVHTPSSNPKLPPPKPLPGTLKRRRPPQPIQQPQRQRPRESYQMGHMRR

SEQ ID NO: 332 - protein, 58 aa, Homo sapiens
XEEGSFSHGSVIDGRFEGFIQTRGGTFYVEPAERYIKDRTLPFHSVIYHEDDISEGLN

SEQ ID NO: 333 - protein, 71 aa, Homo sapiens
MVLLRVLILLLSWAAGMGDRDFDDGVLGLAWVGAPSGSSGGICEKSKLYSDVPTMGKLHSKEKPIKSSSPN

SEQ ID NO: 334 - protein, 125 aa, Homo sapiens
MVLLRVLILLLSWAAGMGGQYGNPLNKYIRHYEGLSYNVDSLHQKHQRAKRAVSHEDQFLRLDFHAHGRHFNLRMKRDTSLFSDEFKVETSNKVLDYDTSHIYTGHIYDYPHKYGPQGGCADHSV

SEQ ID NO: 335 - protein, 59 aa, Homo sapiens
MVLLRVLILLLSWAAGMGAPARWRIPKSSPPPRPAAQTRGKLCCLQLGSCIVLYPARRS

SEQ ID NO: 336 - protein, 115 aa, Homo sapiens
MVLLRVLILLLSWAAGMGGQYGNPLNKYIRHYEGLSYNVDSLHQKHQRAKRAVSHEDQFLRLDFHAHGRDGVLPCCPGWSQTPGLKRSTCLSLPQCWDYRCEPSPMISVFDYPHK

SEQ ID NO: 337 - protein, 148 aa, Homo sapiens
MVLLRVLILLLSWAAGMGDRDFDDGVLGLAWVGAPSGSSGGICEKSKLYSDGKKKSLNTGIITVQNYGSHVPPKVSHITFAHEVGHNFGSPHDSGTECTPGESKNLGQKENGNYIMYARATSGDKLNNNKFSLCSIRNISQVLEKKRN

SEQ ID NO: 338 - protein, 38 aa, Homo sapiens
MVLLRVLILLLSWAAGMGVPTMGKLHSKEKPIKSSSPN

SEQ ID NO: 339 - protein, 452 aa, Homo sapiens
MTLVSGWLLYGWIIAVQTKTKKTLAKPNIRNVVVVDGVRTPFLLSGTSYKDLMPHDLARAALTGLLHRTSVPKEVVDYIIFGTVIQEVKTSNVAREAALGAGFSDKTPAHTVTMACISANQAMTTGVGLIASGQCDVIVAGGVELMSDVPIRHSRKMRKLMLDLNKAKSMGQRLSLISKFRFNFLAPELPAVSEFSTSETMGHSADRLAAAFAVSRLEQDEYALRSHSLAKKAQDEGLLSDVVPFKVPGKDTVTKDNGIRPSSLEQMAKLKPAFIKPYGTVTAANSSFLTDGASAMLIMAEEKALAMGYKPKAYLRDFMYVSQDPKDQLLLGPTYATPKVLEKAGLTMNDIDAFEFHEAFSGQILANFKAMDSDWFAENYMGRKTKVGLPPLEKFNNWGGSLSLGHPFGATGCRLVMAAANRLRKEGGQYGLVAACAAGGQGHAMIVEAYPK

SEQ ID NO: 340 - protein, 260 aa, Homo sapiens
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHQRRKYRSNKGESPVEPAEPCHYSCPREEEGSTIPIQEDYRKPEPACSP

SEQ ID NO: 341 - protein, 819 aa, Homo sapiens
MWGLVRLLLAWLGGWGCMGRLAAPARAWAGSREHPGPALLRTRRSWVWNQFFVIEEYAGPEPVLIGKLHSDVDRGEGRTKYLLTGEGAGTVFVIDEATGNIHVTKSLDREEKAQYVLLAQAVDRASNRPLEPPSEFIIKVQDINDNPPIFPLGPYHATVPEMSNVGTSVIQVTAHDADDPSYGNSAKLVYTVLDGLPFFSVDPQTGVVRTAIPNMDRETQEEFLVVIQAKDMGGHMGGLSGSTTVTVTLSDVNDNPPKFPQSLYQFSVVETAGPGTLVGRLRAQDPDLGDNALMAYSILDGEGSEAFSISTDLQGRDGLLTVRKPLDFESQRSYSFRVEATNTLIDPAYLRRGPFKDVASVRVAVQDAPEPPAFTQAAYHLTVPENKAPGTLVGQISAADLDSPASPIRYSILPHSDPERCFSIQPEEGTIHTAAPLDREARAWHNLTVLATELGWSWGPERGWVPLLVAEWSAPAAPPQRSPVGSAVGIPQDSSAQASRVQVAIQTLDENDNAPQLAEPYDTFVCDSAAPGQLIQVIRALDRDEVGNSSHVSFQGPLGPDANFTVQDNRDGSASLLLPSRPAPPRHAPYLVPIELWDWGQPALSSTATVTVSVCRCQPDGSVASCWPEAHLSAAGLSTGALLAIITCVGALLALVVLFVALRRQKQEALMVLEEEDVRENIITYDDEGGGEEDTEAFDITALQNPDGAAPPAPGPPARRDVLPRARVSRQPRPPGPADVAQLLALRLREADEDPGVPPYDSVQVYGYEGRGSSCGSLSSLGSGSEAGGAPGPAEPLDDWGPLFRTLAELYGAKEPPAP

SEQ ID NO: 342 - protein, 819 aa, Homo sapiens; identical to SEQ ID NO: 341

SEQ ID NO: 343 - protein, 781 aa, Homo sapiens
MWGLVRLLLAWLGGWGCMGRLAAPARAWAGSREHPGPALLRTRRSWVWNQFFVIEEYAGPEPVLIGKLHSDVDRGEGRTKYLLTGEGAGTVFVIDEATGNIHVTKSLDREEKAQYVLLAQAVDRASNRPLEPPSEFIIKVQDINDNPPIFPLGPYHATVPEMSNVGTSVIQVTAHDADDPSYGNSAKLVYTVLDGLPFFSVDPQTGVVRTAIPNMDRETQEEFLVVIQAKDMGGHMGGLSGSTTVTVTLSDVNDNPPKFPQSLYQFSVVETAGPGTLVGRLRAQDPDLGDNALMAYSILDGEGSEAFSISTDLQGRDGLLTVRKPLDFESQRSYSFRVEATNTLIDPAYLRRGPFKDVASVRVAVQDAPEPPAFTQAAYHLTVPENKAPGTLVGQISAADLDSPASPIRYSILPHSDPERCFSIQPEEGTIHTAAPLDREARAWHNLTVLATELDSSAQASRVQVAIQTLDENDNAPQLAEPYDTFVCDSAAPGQLIQVIRALDRDEVGNSSHVSFQGPLGPDANFTVQDNRDGSASLLLPSRPAPPRHAPYLVPIELWDWGQPALSSTATVTVSVCRCQPDGSVASCWPEAHLSAAGLSTGALLAIITCVGALLALVVLFVALRRQKQEALMVLEEEDVRENIITYDDEGGGEEDTEAFDITALQNPDGAAPPAPGPPARRDVLPRARVSRQPRPPGPADVAQLLALRLREADEDPGVPPYDSVQVYGYEGRGSSCGSLSSLGSGSEAGGAPGPAEPLDDWGPLFRTLAELYGAKEPPAP

SEQ ID NO: 344 - protein, 781 aa, Homo sapiens; identical to SEQ ID NO: 343

SEQ ID NO: 345 - protein, 314 aa, Homo sapiens
MWGLVRLLLAWLGGWGCMGRLAAPARAWAGSREHPGPALLRTRRSWVWNQFFVIEEYAGPEPVLIGKLHSDVDRGEGRTKYLLTGEGAGTVFVIDEATGNIHVTKSLDREEKAQYVLLAQAVDRASNRPLEPPSEFIIKIITCVGALLALVVLFVALRRQKQEALMVLEEEDVRENIITYDDEGGGEEDTEAFDITALQNPDGAAPPAPGPPARRDVLPRARVSRQPRPPGPADVAQLLALRLREADEDPGVPPYDSVQVYGYEGRGSSCGSLSSLGSGSEAGGAPGPAEPLDDWGPLFRTLAELYGAKEPPAP

SEQ ID NO: 346 - protein, 45 aa, Homo sapiens
MFRAAAPGQLRRARRGFPMLPRLVSNSCPQVIHLPRPPKVLELQA

SEQ ID NO: 347 - protein, 45 aa, Homo sapiens; identical to SEQ ID NO: 346

SEQ ID NO: 348 - protein, 46 aa, Homo sapiens
MNEAKVKETLRRCGASGDECGRLQYALTCLRKVTGLGGEHKEDSSW

SEQ ID NO: 349 - protein, 248 aa, Homo sapiens
MQTCPLAFPGHVSQALGTLLFLAASLSAQNEGWDSPICTEGVVSVSWGENTVMSCNISNAFSHVNIKLRAHGQESAIFNEVAPGYFSRDGWQLQVQGGVAQLVIKGARDSHAGLYMWHLVGHQRNNRQVTLEVSGAEPQSAPDTGFWPVPAVVTAVFILLVALVMFAWYRCRCSQQRREKKFFLLEPQMKVAALRAGAQQGLSRASAELWTPDSEPTPRPLALVFKPSPLGALELLSPQPLFPYAADP

SEQ ID NO: 350 - protein, 149 aa, Homo sapiens
MQTCPLAFPGHVSQALGTLLFLAASLSAQNEGWDSPICTEGVVSVSWGENTVMSCNISNAFSHVNIKLRAHGQESAIFNEVAPGYFSRDGWQLQVQGGVAQLVIKGARDSHAGLYMWHLVGHQRNNRQVTLEVSGAAVPSNAGRRSSSS

SEQ ID NO: 351 - protein, 178 aa, Homo sapiens
MQTCPLAFPGHVSQALGTLLFLAASLSAQNEGWDSPICTEGVVSVSWGENTVMSCNISNAFSHVNIKLRAHGQESAIFNEVAPGYFSRDGWQLQVQGGVAQLVIKGARDSHAGLYMWHLVGHQRNNRQVTLEVSGAEPQSAPDTGFWPVPAVVTAVFILLVALVMFAWYRCRCSQQRR

SEQ ID NO: 352 - protein, 43 aa, Homo sapiens
MQTCPLAFPGHVSQALGTLLFLAASLSAQNEAACPRAGERHLQ

SEQ ID NO: 353 - protein, 44 aa, Homo sapiens
MQTCPLAFPGHVSQALGTLLFLAASLSAQNEGWDSPICTEGVVS

SEQ ID NO: 354 - protein, 85 aa, Homo sapiens
MQTCPLAFPGHVSQALGTLLFLAASLSAQNEGWDSPICTEGVVSVSWGENTVMSCNISNAFSHVNIKLRAHGQESAIFNEVAPGY

SEQ ID NO: 355 - protein, 170 aa, Homo sapiens
MQTCPLAFPGHVSQALGTLLFLAASLSAQNEGWDSPICTEGVVSVSWGENTVMSCNISNAFSHVNIKLRAHGQESAIFNEVAPGYFSRDGWQLQVQGGVAQLVIKGARDSHAGLYMWHLVGHQRNNRQVTLEVSEPRGLKDRASAQLLGPGTSRGAEPQSAPDTGFWPVP

SEQ ID NO: 356 - protein, 147 aa, Homo sapiens
MQTCPLAFPGHVSQALGTLLFLAASLSAQNEGWDSPICTEGVVSVSWGENTVMSCNISNAFSHVNIKLRAHGQESAIFNEVAPGYFSRDGWQLQVQGGVAQLVIKGARDSHAGLYMWHLVGHQRNNRQVTLEVSEPRGLKDRASAQL

SEQ ID NO: 357 - protein, 165 aa, Homo sapiens
MDAPRRDMELLSNSLAAYAHIRANPESFGLYFVLGVCFGLLLTLCLLVISISWAPRPRPRGPAQRRDPRSSTLEPEDDDEDEEDTVTRLGPDDTLPGPELSAEPDGPLNVNVFTSAEELERAQRLEERERILREIWRTGQPDLLGTGTLGPSPTATGTLGRMHYY

SEQ ID NO: 358 - protein, 639 aa, Homo sapiens
MNLEKLSKPELLTLFSILEGELEARDLVIEALKAQHRDTFIEERYGKYNISDPLMALQRDFETLKEKNDGEKQPVCTNPLSILKVVMKQCKNMQERMLSQLAAAESRHRKVILDLEEERQRHAQDTAEGDDVTYMLEKERERLTQQLEFEKSQVKKFEKEQKKLSSQLEEERSRHKQLSSMLVLECKKATNKAAEEGQKAGELSLKLEKEKSRVSKLEEELAAERKRGLQTEAQVEKQLSEFDIEREQLRAKLNREENRTKTLKEEMESLKKIVKDLEASHQHSSPNEQLKKPVTVSKGTATEPLMLMSVFCQTESFPAERTHGSNIAKMTNTGLPGPATPAYSYAKTNGHCDPEIQTTRELTAGNNVENQVPPREKSVALAQEKPVENGGCPVGIETPVPMPSPLSSSGSSLSPSSTASSSLTSSPCSSPVLTKRLLGSSASSPGYQSSYQVGINQRFHAARHKFQSQADQDQQASGLQSPPSRDLSPTLIDNSAAKQLARNTVTQVLSRFTSQQGPIKPVSPNSSPFGTDYRNLANTANPRGDTSHSPTPGKVSSPLSPLSPGIKSPTIPRAERGNPPPIPPKKPGLTPSPSATTPLTKTHSQAASLTTAEDLASSCSSNTVVANGKDVELLLPTSS

SEQ ID NO: 359 - protein, 271 aa, Homo sapiens
MNLEKLSKPELLTLFSILEGELEARDLVIEALKAQHRDTFIEERYGKYNISDPLMALQRDFETLKEKNDGEKQPVCTNPLSILKVVMKQCKNMQERMLSQLAAAESRHRKVILDLEEERQRHAQDTAEGDDVTYMLEKERERLTQQLEFEKSQVKKFEKEQKKLSSQLEEERSRHKQLSSMLVLECKKATNKAAEEGQKAGELSLKLEKEKSRVSKLEEELAAERKRGLQTEAQVEKQLSEFDIEREQLRAKLNREENRTKTLKEEMESLK

SEQ ID NO: 360 - protein, 396 aa, Homo sapiens
MSATDRMGPRAVPGLRLALLLLLVLGTPKSGVQGQEGLDFPEYDGVDRVINVNAKNYKNVFKKYEVLALLYHEPPEDDKASQRQFEMEELILELAAQVLEDKGVGFGLVDSEKDAAVAKKLGLTEVDSMYVFKGDEVIEYDGEFSADTIVEFLLDVLEDPVELIEGERELQAFENIEDEIKLIGYFKSKDSEHYKAFEDAAEEFHPYIPFFATFDSKVAKKLTLKLNEIDFYEAFMEEPVTIPDKPNSEEEIVNFVEEHRRSTLRKLKPESMYETWEDDMDGIHIVAFAEEADPDGFEFLETLKAVAQDNTENPDLSIIWIDPDDFPLLVPYWEKTFDIDLSAPQIGVVNVTDADSVWMEMDDEEDLPSAEELEDWLEDVLEGEINTEDDDDDDDD

SEQ ID NO: 361 - protein, 188 aa, Homo sapiens
MDVNIAPLRAWDDFFPGSDRFARPDFRDISKWNNRVVSNLLYYQTNYLVVAAMMISIVGFLSPFNMILGGIVVVLVFTGFVWAAHNKDVLRRMKKRYPTTFVMVVMLASYFLISMFGGVMVFVFGITFPLLLMFIHASLRLRNLKNKLENKMEGIGLKRTPMGIVLDALEQQEEGINRLTDYISKVKE

SEQ ID NO: 362 - protein, 88 aa, Homo sapiens
MDVNIAPLRAWDDFFPGSDRFARPDFRDISKWNNRVVSNLLYYQTNYLVVAAMMISIVGFLSPFNPPIVSEVSRVFMKSNGKWITRFL

SEQ ID NO: 363 - protein, 93 aa, Homo sapiens
MDVNIAPLRAWDDFFPGSDRFARPDFRDISKWNNRVVSNLLYYQTNYLVVAAMMISIVGLECNGVISAHCNLHLLSSSDSPASASQVAGITGF

SEQ ID NO: 364 - protein, 59 aa, Homo sapiens
MDVNIAPLRAWDDFFPGSDRFARPDFRDISKWNNRVVSNLLYYQTNYLVVAAMMISIVG

SEQ ID NO: 365 - protein, 357 aa, Homo sapiens
MEKYVAAMVLSAAGDALGYYNGKWEFLQDGEKIHRQLAQLGGLDALDVGRWRVSDDTVMHLATAEALVEAGKAPKLTQLYYLLAKHYQDCMEDMDGRAPGGASVHNAMQLKPGKPNGWRIPFNSHEGGCGAAMRAMCIGLRFPHHSQLDTLIQVSIESGRMTHHHPTGYLGALASALFTAYAVNSRPPLQWGKGLMELLPEAKKYIVQSGYFVEENLQHWSYFQTKWENYLKLRGILDGESAPTFPESFGVKERDQFYTSLSYSGWGGSSGHDAPMIAYDAVLAAGDSWKELAHRAFFHGGDSDSTAAIAGCWWGVMYGFKGVSPSNYEKLEYRNRLEETARALYSLGSKEDTVISL

SEQ ID NO: 366 - protein, 357 aa, Homo sapiens; identical to SEQ ID NO: 365

SEQ ID NO: 367 - protein, 357 aa, Homo sapiens; identical to SEQ ID NO: 365

SEQ ID NO: 368 - protein, 357 aa, Homo sapiens; identical to SEQ ID NO: 365

SEQ ID NO: 369 - protein, 125 aa, Homo sapiens
MEKYVAAMVLSAAGDALGYYNGKWEFLQDGEKIHRQLAQLGGLDALDVGRWRVSDDTVMHLATAEALVEAGKAPKLTQLYYLLAKHYQDCMEDMDGRAPGGASVHNAMQLKPGKPNGWRIPFNSH

SEQ ID NO: 370 - protein, 974 aa, Homo sapiens
MAGRVPSLLVLLVFPSSCLAFRSPLSVFKRFKETTRPFSNECLGTTRPVVPIDSSDFALDIRMPGVTPKQSDTYFCMSMRIPVDEEAFVIDFKPRASMDTVHHMLLFGCNMPSSTGSYWFCDEGTCTDKANILYAWARNAPPTRLPKGVGFRVGGETGSKYFVLQVHYGDISAFRDNNKDCSGVSLHLTRLPQPLIAGMYLMMSVDTVIPAGEKVVNSDISCHYKNYPMHVFAYRVHTHHLGKVVSGYRVRNGQWTLIGRQSPQLPQAFYPVGHPVDVSFGDLLAARCVFTGEGRTEATHIGGTSSDEMCNLYIMYYMEAKHAVSFMTCTQNVAPDMFRTIPPEANIPIPVKSDMVMMHEHHKETEYKDKIPLLQQPKREEEEVLDQGDFYSLLSKLLGEREDVVHVHKYNPTEKAESESDLVAEIANVVQKKDLGRSDAREGAEHERGNAILVRDRIHKFHRLVSTLRPPESRVFSLQQPPPGEGTWEPEHTGDFHMEEALDWPGVYLLPGQVSGVALDPKNNLVIFHRGDHVWDGNSFDSKFVYQQIGLGPIEEDTILVIDPNNAAVLQSSGKNLFYLPHGLSIDKDGNYWVTDVALHQVFKLDPNNKEGPVLILGRSMQPGSDQNHFCQPTDVAVDPGTGAIYVSDGYCNSRIVQFSPSGKFITQWGEESSGSSPLPGQFTVPHSLALVPLLGQLCVADRENGRIQCFKTDTKEFVREIKHSSFGRNVFAISYIPGLLFAVNGKPHFGDQEPVQGFVMNFSNGEIIDIFKPVRKHFDMPHDIVASEDGTVYIGDAHTNTVWKFTLTEKLEHRSVKKAGIEVQEIKEAEAVVETKMENKPTSSELQKMQEKQKLIKEPGSGVPVVLITTLLVIPVVVLLAIAIFIRWKKSRAFGADSEHKLETSSGRVLGRFRGKGSGGLNLGNFFASRKGYSRKGFDRLSTEGSDQEKEDDGSESEEEYSAPLPALAPSSS

SEQ ID NO: 371 - protein, 175 aa, Homo sapiens
MAGRVPSLLVLLVFPSSCLAFRSPLSVFKRFKETTRPFSNECLGTTRPVVPIDSSDFALDIRMPGVTPKQLTSSLEPAWILSITCYFLDAICLHPLEVTGFVMKEPVQIKPIFCMPGREMLPLPGSPKVLDSELEERLEVNTLYYRYTMGILVLLEIITRTVLVCPYTSHVCHSL

SEQ ID NO: 372 - protein, 887 aa, Homo sapiens
MAGRVPSLLVLLVFPSSCLAFRSPLSVFKRFKETTRPFSNECLGTTRPVVPIDSSDFALDIRMPGVTPKQSDTYFCMSMRIPVDEEAFVIDFKPRASMDTVHHMLLFGCNMPSSTGSYWFCDEGTCTDKANILYAWARNAPPTRLPKGVGFRVGGETGSKYFVLQVHYGDISAFRDNNKDCSGVSLHLTRLPQPLIAGMYLMMSVDTVIPAGEKVVNSDISCHYKNYPMHVFAYRVHTHHLGKVVSGYRVRNGQWTLIGRQSPQLPQAFYPVGHPVDVSFGDLLAARCVFTGEGRTEATHIGGTSSDEMCNLYIMYYMEAKHAVSFMTCTQNVAPDMFRTIPPEANIPIPVKSDMVMMHEHHKETEYKDKIPLLQQPKREEEEVLDQGDFYSLLSKLLGEREDVVHVHKYNPTEKAESESDLVAEIANVVQKKDLGRSDAREGAEHERGNAILVRDRIHKFHRLVSTLRPPESRVFSLQQPPPGEGTWEPEHTGDFHMEEALDWPGVYLLPGQVSGVALDPKNNLVIFHRGDHVWDGNSFDSKFVYQQIGLGPIEEDTILVIDPNNAAVLQSSGKNLFYLPHGLSIDKDGNYWVTDVALHQVFKLDPNNKEGPVLILGRSMQPGSDQNHFCQPTDVAVDPGTGAIYVSDGYCNSRIVQFSPSGKFITQWGEESSGSSPLPGQFTVPHSLALVPLLGQLCVADRENGRIQCFKTDTKEFVREIKHSSFGRNVFAISYIPGLLFAVNGKPHFGDQEPVQGFVMNFSNGEIIDIFKPVRKHFDMPHDIVASEDGTVYIGDAHTNTVWKFTLTEKLEHRSVKKAGIEVQEIKGKGSGGLNLGNFFASRKGYSRKGFDRLSTEGSDQEKEDDGSESEEEYSAPLPALAPSSS

SEQ ID NO: 373 - protein, 866 aa, Homo sapiens
MAGRVPSLLVLLVFPSSCLAFRSPLSVFKRFKETTRPFSNECLGTTRPVVPIDSSDFALDIRMPGVTPKQSDTYFCMSMRIPVDEEAFVIDFKPRASMDTVHHMLLFGCNMPSSTGSYWFCDEGTCTDKANILYAWARNAPPTRLPKGVGFRVGGETGSKYFVLQVHYGDISAFRDNNKDCSGVSLHLTRLPQPLIAGMYLMMSVDTVIPAGEKVVNSDISCHYKNYPMHVFAYRVHTHHLGKVVSGYRVRNGQWTLIGRQSPQLPQAFYPVGHPVDVSFGDLLAARCVFTGEGRTEATHIGGTSSDEMCNLYIMYYMEAKHAVSFMTCTQNVAPDMFRTIPPEANIPIPVKSDMVMMHEHHKETEYKDKIPLLQQPKREEEEVLDQDFHMEEALDWPGVYLLPGQVSGVALDPKNNLVIFHRGDHVWDGNSFDSKFVYQQIGLGPIEEDTILVIDPNNAAVLQSSGKNLFYLPHGLSIDKDGNYWVTDVALHQVFKLDPNNKEGPVLILGRSMQPGSDQNHFCQPTDVAVDPGTGAIYVSDGYCNSRIVQFSPSGKFITQWGEESSGSSPLPGQFTVPHSLALVPLLGQLCVADRENGRIQCFKTDTKEFVREIKHSSFGRNVFAISYIPGLLFAVNGKPHFGDQEPVQGFVMNFSNGEIIDIFKPVRKHFDMPHDIVASEDGTVYIGDAHTNTVWKFTLTEKLEHRSVKKAGIEVQEIKEAEAVVETKMENKPTSSELQKMQEKQKLIKEPGSGVPVVLITTLLVIPVVVLLAIAIFIRWKKSRAFGDSEHKLETSSGRVLGRFRGKGSGGLNLGNFFASRKGYSRKGFDRLSTEGSDQEKEDDGSESEEEYSAPLPALAPSSS

SEQ ID NO: 374 - protein, 973 aa, Homo sapiens [sequence omitted]

SEQ ID NO: 375 - protein, 905 aa, Homo sapiens
MAGRVPSLLVLLVFPSSCLAFRSPLSVFKRFKETTRPFSNECLGTTRPVVPIDSSDFALDIRMPGVTPKQSDTYFCMSMRIPVDEEAFVIDFKPRASMDTVHHMLLFGCNMPSSTGSYWFCDEGTCTDKANILYAWARNAPPTRLPKGVGFRVGGETGSKYFVLQVHYGDISAFRDNNKDCSGVSLHLTRLPQPLIAGMYLMMSVDTVIPAGEKVVNSDISCHYKNYPMHVFAYRVHTHHLGKVVSGYRVRNGQWTLIGRQSPQLPQAFYPVGHPVDVSFGDLLAARCVFTGEGRTEATHIGGTSSDEMCNLYIMYYMEAKHAVSFMTCTQNVAPDMFRTIPPEANIPIPVKSDMVMMHEHHKETEYKDKIPLLQQPKREEEEVLDQGDFYSLLSKLLGEREDVVHVHKYNPTEKAESESDLVAEIANVVQKKDLGRSDAREGAEHERGNAILVRDRIHKFHRLVSTLRPPESRVFSLQQPPPGEGTWEPEHTGDFHMEEALDWPGVYLLPGQVSGVALDPKNNLVIFHRGDHVWDGNSFDSKFVYQQIGLGPIEEDTILVIDPNNAAVLQSSGKNLFYLPHGLSIDKDGNYWVTDVALHQVFKLDPNNKEGPVLILGRSMQPGSDQNHFCQPTDVAVDPGTGAIYVSDGYCNSRIVQFSPSGKFITQWGEESSGSSPLPGQFTVPHSLALVPLLGQLCVADRENGRIQCFKTDTKEFVREIKHSSFGRNVFAISYIPGLLFAVNGKPHFGDQEPVQGFVMNFSNGEIIDIFKPVRKHFDMPHDIVASEDGTVYIGDAHTNTVWKFTLTEKLEHRSVKKAGIEVQEIKDSEHKLETSSGRVLGRFRGKGSGGLNLGNFFASRKGYSRKGFDRLSTEGSDQEKEDDGSESEEEYSAPLPALAPSSS

SEQ ID NO: 376 - protein, 250 aa, Homo sapiens
XRIQCFKTDTKEFVREIKHSSFGRNVFAISYIPGLLFAVNGKPHFGDQEPVQGFVMNFSNGEIIDIFKPVRKHFDMPHDIVASEDGTVYIGDAHTNTVWKFTLTEKLEHRSVKKAGIEVQEIKEAEAVVETKMENKPTSSELQKMQEKQKLIKEPGSGVPVVLITTLLVIPVVVLLAIAIFIRWKKSRAFGGKGSGGLNLGNFFASRKGYSRKGFDRLSTEGSDQEKEDDGSESEEEYSAPLPALAPSSS

SEQ ID NO: 377 - protein, 29 aa, Homo sapiens
MAGRVPSLLVLLVFPSSCLAFRSPLSVFK

SEQ ID NO: 378 - protein, 28 aa, Homo sapiens
MAGRVPSLLVLLVFPSSCLAFRSPLSVF

SEQ ID NO: 379 - protein, 53 aa, Homo sapiens
MAGRVPSLLVLLVFPSSCLAFRSPLSVFKRFKETTRPFSNECLGTTRPVVPID

SEQ ID NO: 380 - protein, 42 aa, Homo sapiens
MAGRVPSLLVLLVFPSSCLAFRSPLSVFKRFKETTRPFSNEC

SEQ ID NO: 381 - protein, 68 aa, Homo sapiens
MAGRVPSLLVLLVFPSSCLAFRSPLSVFKRFKETTRPFSNECLGTTRPVVPIDSSDFALDIRMPGVTP

SEQ ID NO: 382 - protein, 25 aa, Homo sapiens
MAGRVPSLLVLLVFPSSCLAFRSPL

SEQ ID NO: 383 - protein, 691 aa, Homo sapiens
MMRNHRIASSLCGDQVFSKKKKKKKKNNMAAKEKLEAVLNVALRVPSIMLLDVLYRWDVSSFFQQIQRSSLSNNPLFQYKYLALNMHYVGYILSVVLLTLPRQHLVQLYLYFLTALLLYAGHQISRDYVRSELEFAYEGPMYLEPLSMNRFTTALIGQLVVCTLCSCVMKTKQIWLFSAHMLPLLARLCLVPLETIVIINKFAMIFTGLEVLYFLGSNLLVPYNLAKSAYRELVQVVEVYGLLALGMSLWNQLVVPVLFMVFWLVLFALQIYSYFSTRDQPASRERLLFLFLTSIAECCSTPYSLLGLVFTVSFVALGVLTLCKFYLQGYRAFMNDPAMNRGMTEGVTLLILAVQTGLIELQVVHRAFLLSIILFIVVASILQSMLEIADPIVLALGASRDKSLWKHFRAVSLCLFLLVFPAYMAYMICQFFHMDFWLLIIISSSILTSLQVLGTLFIYVLFMVEEFRKEPVENMDDVIYYVNGTYRLLEFLVALCVVAYGVSETIFGEWTVMGSMIIFIHSYYNVWLRAQLGWKSFLLRRDAVNKIKSLPIATKEQLEKHNDICAICYQDMKSAVITPCSHFFHAGCLKKWLYVQETCPLCHCHLKNSSQLPGLGTEPVLQPHAGAEQNVMFQEGTEPPGQEHTPGTRIQEGSRDNNEYIARRPDNQEGAFDPKEYPHSAKDEAHPVESA

SEQ ID NO: 384 - protein, 663 aa, Homo sapiens
MAAKEKLEAVLNVALRVPSIMLLDVLYRWDVSSFFQQIQRSSLSNNPLFQYKYLALNMHYVGYILSVVLLTLPRQHLVQLYLYFLTALLLYAGHQISRDYVRSELEFAYEGPMYLEPLSMNRFTTALIGQLVVCTLCSCVMKTKQIWLFSAHMLPLLARLCLVPLETIVIINKFAMIFTGLEVLYFLGSNLLVPYNLAKSAYRELVQVVEVYGLLALGMSLWNQLVVPVLFMVFWLVLFALQIYSYFSTRDQPASRERLLFLFLTSIAECCSTPYSLLGLVFTVSFVALGVLTLCKFYLQGYRAFMNDPAMNRGMTEGVTLLILAVQTGLIELQVVHRAFLLSIILFIVVASILQSMLEIADPIVLALGASRDKSLWKHFRAVSLCLFLLVFPAYMAYMICQFFHMDFWLLIIISSSILTSLQVLGTLFIYVLFMVEEFRKEPVENMDDVIYYVNGTYRLLEFLVALCVVAYGVSETIFGEWTVMGSMIIFIHSYYNVWLRAQLGWKSFLLRRDAVNKIKSLPIATKEQLEKHNDICAICYQDMKSAVITPCSHFFHAGCLKKWLYVQETCPLCHCHLKNSSQLPGLGTEPVLQPHAGAEQNVMFQEGTEPPGQEHTPGTRIQEGSRDNNEYIARRPDNQEGAFDPKEYPHSAKDEAHPVESA

SEQ ID NO: 385 - protein, 693 aa, Homo sapiens
MHRDRISPSNSPTWSLQVFSKKKKKKKKNNMAAKEKLEAVLNVALRVPSIMLLDVLYRWDVSSFFQQIQRSSLSNNPLFQYKYLALNMHYVGYILSVVLLTLPRQHLVQLYLYFLTALLLYAGHQISRDYVRSELEFAYEGPMYLEPLSMNRFTTALIGQLVVCTLCSCVMKTKQIWLFSAHMLPLLARLCLVPLETIVIINKFAMIFTGLEVLYFLGSNLLVPYNLAKSAYRELVQVVEVYGLLALGMSLWNQLVVPVLFMVFWLVLFALQIYSYFSTRDQPASRERLLFLFLTSIAECCSTPYSLLGLVFTVSFVALGVLTLCKFYLQGYRAFMNDPAMNRGMTEGVTLLILAVQTGLIELQVVHRAFLLSIILFIVVASILQSMLEIADPIVLALGASRDKSLWKHFRAVSLCLFLLVFPAYMAYMICQFFHMDFWLLIIISSSILTSLQVLGTLFIYVLFMVEEFRKEPVENMDDVIYYVNGTYRLLEFLVALCVVAYGVSETIFGEWTVMGSMIIFIHSYYNVWLRAQLGWKSFLLRRDAVNKIKSLPIATKEQLEKHNDICAICYQDMKSAVITPCSHFFHAGCLKKWLYVQETCPLCHCHLKNSSQLPGLGTEPVLQPHAGAEQNVMFQEGTEPPGQEHTPGTRIQEGSRDNNEYIARRPDNQEGAFDPKEYPHSAKDEAHPVESA

SEQ ID NO: 386 - protein, 663 aa, Homo sapiens; identical to SEQ ID NO: 384

SEQ ID NO: 387 - protein, 677 aa, Homo sapiens
MVFSKKKKKKKKNNMAAKEKLEAVLNVALRVPSIMLLDVLYRWDVSSFFQQIQRSSLSNNPLFQYKYLALNMHYVGYILSVVLLTLPRQHLVQLYLYFLTALLLYAGHQISRDYVRSELEFAYEGPMYLEPLSMNRFTTALIGQLVVCTLCSCVMKTKQIWLFSAHMLPLLARLCLVPLETIVIINKFAMIFTGLEVLYFLGSNLLVPYNLAKSAYRELVQVVEVYGLLALGMSLWNQLVVPVLFMVFWLVLFALQIYSYFSTRDQPASRERLLFLFLTSIAECCSTPYSLLGLVFTVSFVALGVLTLCKFYLQGYRAFMNDPAMNRGMTEGVTLLILAVQTGLIELQVVHRAFLLSIILFIVVASILQSMLEIADPIVLALGASRDKSLWKHFRAVSLCLFLLVFPAYMAYMICQFFHMDFWLLIIISSSILTSLQVLGTLFIYVLFMVEEFRKEPVENMDDVIYYVNGTYRLLEFLVALCVVAYGVSETIFGEWTVMGSMIIFIHSYYNVWLRAQLGWKSFLLRRDAVNKIKSLPIATKEQLEKHNDICAICYQDMKSAVITPCSHFFHAGCLKKWLYVQETCPLCHCHLKNSSQLPGLGTEPVLQPHAGAEQNVMFQEGTEPPGQEHTPGTRIQEGSRDNNEYIARRPDNQEGAFDPKEYPHSAKDEAHPVESA

SEQ ID NO: 388 - protein, 680 aa, Homo sapiens
MAEVVFSKKKKKKKKNNMAAKEKLEAVLNVALRVPSIMLLDVLYRWDVSSFFQQIQRSSLSNNPLFQYKYLALNMHYVGYILSVVLLTLPRQHLVQLYLYFLTALLLYAGHQISRDYVRSELEFAYEGPMYLEPLSMNRFTTALIGQLVVCTLCSCVMKTKQIWLFSAHMLPLLARLCLVPLETIVIINKFAMIFTGLEVLYFLGSNLLVPYNLAKSAYRELVQVVEVYGLLALGMSLWNQLVVPVLFMVFWLVLFALQIYSYFSTRDQPASRERLLFLFLTSIAECCSTPYSLLGLVFTVSFVALGVLTLCKFYLQGYRAFMNDPAMNRGMTEGVTLLILAVQTGLIELQVVHRAFLLSIILFIVVASILQSMLEIADPIVLALGASRDKSLWKHFRAVSLCLFLLVFPAYMAYMICQFFHMDFWLLIIISSSILTSLQVLGTLFIYVLFMVEEFRKEPVENMDDVIYYVNGTYRLLEFLVALCVVAYGVSETIFGEWTVMGSMIIFIHSYYNVWLRAQLGWKSFLLRRDAVNKIKSLPIATKEQLEKHNDICAICYQDMKSAVITPCSHFFHAGCLKKWLYVQETCPLCHCHLKNSSQLPGLGTEPVLQPHAGAEQNVMFQEGTEPPGQEHTPGTRIQEGSRDNNEYIARRPDNQEGAFDPKEYPHSAKDEAHPVESA

SEQ ID NO: 389 - protein, 663 aa, Homo sapiens; identical to SEQ ID NO: 384

SEQ ID NO: 390 - protein, 185 aa, Homo sapiens
MAGPRPRWRDQLLFMSIIVLVIVVICLMFYALLWEAGNLTDLPNLRIGFYNFCLWNEDTSTLQCHQFPELEALGVPRVGLGLARLGVYGSLVLTLFAPQPLLLAQCNSDERAWRLAVGFLAVSSVLLAGGLGLFLSYVWKWVRLSLPGPGFLALGSAQALLILLLIAMAVFPLRAERAESKLESC

SEQ ID NO: 391 - protein, 486 aa, Homo sapiens
MKGRRRRRREYCKFALLLVLYTLVLLLVPSVLDGGRDGDKGAEHCPGLQRSLGVWSLEAAAAGEREQGAEARAAEEGGANQSPRFPSNLSGAVGEAVSREKQHIYVHATWRTGSSFLGELFNQHPDVFYLYEPMWHLWQALYPGDAESLQGALRDMLRSLFRCDFSVLRLYAPPGDPAARAPDTANLTTAALFRWRTNKVICSPPLCPGAPRARAEVGLVEDTACERSCPPVAIRALEAECRKYPVVVIKDVRLLDLGVLVPLLRDPGLNLKVVQLFRDPRAVHNSRLKSRQGLLRESIQVLRTRQRGDRFHRVLLAHGVGARPGGQSRALPAAPRADFFLTGALEVICEAWLRDLLFARGAPAWLRRRYLRLRYEDLVRQPRAQLRRLLRFSGLRALAALDAFALNMTRGAAYGADRPFHLSARDAREAVHAWRERLSREQVRQVEAACAPAMRLLAYPRSGEEGDAEQPREGETPLEMDADGAT

SEQ ID NO: 392 - protein, 494 aa, Homo sapiens
MLTSKGQGFLHGGLCLWLCVFTPFFKGCVGCATEERLFHKLFSHYNQFIRPVENVSDPVTVHFEVAITQLANVDEVNQIMETNLWLRHIWNDYKLRWDPMEYDGIETLRVPADKIWKPDIVLYNNAVGDFQVEGKTKALLKYNGMITWTPPAIFKSSCPMDITFFPFDHQNCSLKFGSWTYDKAEIDLLIIGSKVDMNDFWENSEWEIIDASGYKHDIKYNCCEEIYTDITYSFYIRRLPMFYTINLIIPCLFISFLTVLVFYLPSDCGEKVTLCISVLLSLTVFLLVITETIPSTSLVVPLVGEYLLFTMIFVTLSIVVTVFVLNIHYRTPTTHTMPRWVKTVFLKLLPQVLLMRWPLDKTRGTGSDAVPRGLARRPAKGKLASHGEPRHLKECFHCHKSNELATSKRRLSHQPLQWVVENSEHSPEVEDVINSVQFIAENMKSHNETKEVEDDWKYVAMVVDRVFLWVFIIVCVFGTAGLFLQPLLGNTGKS

SEQ ID NO: 393 - protein, 111 aa, Homo sapiens
METNLWLRHIWNDYKLRWDPMEYDGIETLRVPADKIWKPDIVLYNNAVGDFQVEGKTKALLKYNGMITWTPPAIFKSSCPMDITFFPFDHQNCSLKFGSWTYDKAEIDLLI

SEQ ID NO: 394 - protein, 479 aa, Homo sapiens
MLTSKGQGFLHGGLCLWLCVFTPFFKGCVGCATEERLFHKLFSHYNQFIRPVENVSDPVTVHFEVAITQLANVIWNDYKLRWDPMEYDGIETLRVPADKIWKPDIVLYNNAVGDFQVEGKTKALLKYNGMITWTPPAIFKSSCPMDITFFPFDHQNCSLKFGSWTYDKAEIDLLIIGSKVDMNDFWENSEWEIIDASGYKHDIKYNCCEEIYTDITYSFYIRRLPMFYTINLIIPCLFISFLTVLVFYLPSDCGEKVTLCISVLLSLTVFLLVITETIPSTSLVVPLVGEYLLFTMIFVTLSIVVTVFVLNIHYRTPTTHTMPRWVKTVFLKLLPQVLLMRWPLDKTRGTGSDAVPRGLARRPAKGKLASHGEPRHLKECFHCHKSNELATSKRRLSHQPLQWVVENSEHSPEVEDVINSVQFIAENMKSHNETKEVEDDWKYVAMVVDRVFLWVFIIVCVFGTAGLFLQPLLGNTGKS

SEQ ID NO: 395 - protein, 1337 aa, Homo sapiens
MKPAAREARLPPRSPGLRWALPLLLLLLRLGQILCAGGTPSPIPDPSVATVATGENGITQISSTAESFHKQNGTGTPQVETNTSEDGESSGANDSLRTPEQGSNGTDGASQKTPSSTGPSPVFDIKAVSISPTNVILTWKSNDTAASEYKYVVKHKMENEKTITVVHQPWCNITGLRPATSYVFSITPGIGNETWGDPRVIKVITEPIPVSDLRVALTGVRKAALSWSNGNGTASCRVLLESIGSHEELTQDSRLQVNISGLKPGVQYNINPYLLQSNKTKGDPLGTEGGLDASNTERSRAGSPTAPVHDESLVGPVDPSSGQQSRDTEVLLVGLEPGTRYNATVYSQAANGTEGQPQAIEFRTNAIQVFDVTAVNISATSLTLIWKVSDNESSSNYTYKIHVAGETDSSNLNVSEPRAVIPGLRSSTFYNITVCPVLGDIEGTPGFLQVHTPPVPVSDFRVTVVSTTEIGLAWSSHDAESFQMHITQEGAGNSRVEITTNQSIIIGGLFPGTKYCFEIVPKGPNGTEGASRTVCNRTVPSAVFDIHVVYVTTTEMWLDWKSPDGASEYVYHLVIESKHGSNHTSTYDKAITLQGLIPGTLYNITISPEVDHVWGDPNSTAQYTRPSNVSNIDVSTNTTAATLSWQNFDDASPTYSYCLLIEKAGNSSNATQVVTDIGITDATVTELIPGSSYTVEIFAQVGDGIKSLEPGRKSFCTDPASMASFDCEVVPKEPALVLKWTCPPGANAGFELEVSSGAWNNATHLESCSSENGTEYRTEVTYLNFSTSYNISITTVSCGKMAAPTRNTCTTGITDPPPPDGSPNITSVSHNSVKVKFSGFEASHGPIKAYAVILTTGEAGHPSADVLKYTYEDFKKGASDTYVTYLIRTEEKGRSQSLSEVLKYEIDVGNESTTLGYYNGKLEPLGSYRACVAGFTNITFHPQNKGLIDGAESYVSFSRYSDAVSLPQDPGVICGAVFGCIFGALVIVTVGGFIFWRKKRKDAKNNEVSFSQIKPKKSKLIRVENFEAYFKKQQADSNCGFAEEYEDLKLVGISQPKYAAELAENRGKNRYNNVLPYDISRVKLSVQTHSTDDYINANYMPGYHSKKDFIATQGPLPNTLKDFWRMVWEKNVYAIIMLTKCVEQGRTKCEEYWPSKQAQDYGDITVAMTSEIVLPEWTIRDFTVKNIQTSESHPLRQFHFTSWPDHGVPDTTDLLINFRYLVRDYMKQSPPESPILVHCSAGVGRTGTFIAIDRLIYQIENENTVDVYGIVYDLRMHRPLMVQTEDQYVFLNQCVLDIVRSQKDSKVDLIYQNTTAMTIYENLAPVTTFGKTNGYIA

SEQ ID NO: 396 - protein, 539 aa, Homo sapiens
MKPAAREARLPPRSPGLRWALPLLLLLLRLGQILCAGGTPSPIPDPSVATVATGENGITQISSTAESFHKQNGTGTPQVETNTSEDGESSGANDSLRTPEQGSNGTDGASQKTPSSTGPSPVFDIKAVSISPTNVILTWKSNDTAASEYKYVVKHKMENEKTITVVHQPWCNITGLRPATSYVFSITPGIGNETWGDPRVIKVITEPIPVSDLRVALTGVRKAALSWSNGNGTASCRVLLESIGSHEELTQDSRLQVNISGLKPGVQYNINPYLLQSNKTKGDPLGTEGGLDASNTERSRAGSPTAPVHDESLVGPVDPSSGQQSRDTEVLLVGLEPGTRYNATVYSQAANGTEGQPQAIEFRTNAIQVFDVTAVNISATSLTLIWKVSDNESSSNYTYKIHVAGETDSSNLNVSEPRAVIPGLRSSTFYNITVCPVLGDIEGTPGFLQVHTPPVPVSDFRVTVVSTTEIGLAWSSHDAESFQMHITQEGAGNSRVEITTNQSIIIGGLFPGTKYCFEIVPKGPNGTEGASRTVCNRTG

SEQ ID NO: 397 - protein, 147 aa, Homo sapiens [sequence omitted]

SEQ ID NO: 398 - protein, 120 aa, Homo sapiens
MKPAAREARLPPRSPGLRWALPLLLLLLRLGQILCAGGRPSPVFDIKAVSISPTNVILTWKSNDTAASEYKYVVKHKMENEKTITVVHQPWCNITGLRPATSYVFSITPGIGNETWGDPR

SEQ ID NO: 399 - protein, 1338 aa, Homo sapiens [sequence omitted]

SEQ ID NO: 400 - protein, 1342 aa, Homo sapiens
MKPAAREARLPPRSPGLRWALPLLLLLLRLGQILCAGGTPSPIPDPSVATVATGENGITQISSTAESFHKQNGTGTPQVETNTSEDGESSGANDSLRTPEQGSNGTDGASQKTPSSTGPSPVFDIKAVSISPTNVILTWKSNDTAASEYKYVVKHKMENEKTITVVHQPWCNITGLRPATSYVFSITPGIGNETWGDPRVIKVITEPIPVSDLRVALTGVRKAALSWSNGNGTASCRVLLESIGSHEELTQDSRLQVNISGLKPGVQYNINPYLLQSNKTKGDPLGTEGGLDASNTERSRAGSPTAPVHDESLVGPVDPSSGQQSRDTEVLLVGLEPGTRYNATVYSQAANGTEGQPQAIEFRTNAIQVFDVTAVNISATSLTLIWKVSDNESSSNYTYKIHVAGETDSSNLNVSEPRAVIPGLRSSTFYNITVCPVLGDIEGTPGFLQVHTPPVPVSDFRVTVVSTTEIGLAWSSHDAESFQMHITQEGAGNSRVEITTNQSIIIGGLFPGTKYCFEIVPKGPNGTEGASRTVCNRTVPSAVFDIHVVYVTTTEMWLDWKSPDGASEYVYHLVIESKHGSNHTSTYDKAITLQGLIPGTLYNITISPEVDHVWGDPNSTAQYTRPSNVSNIDVSTNTTAATLSWQNFDDASPTYSYCLLIEKAGNSSNATQVVTDIGITDATVTELIPGSSYTVEIFAQVGDGIKSLEPGRKSFCTDPASMASFDCEVVPKEPALVLKWTCPPGANAGFELEVSSGAWNNATHLESCSSENGTEYRTEVTYLNFSTSYNISITTVSCGKMAAPTRNTCTTGITDPPPPDGSPNITSVSHNSVKVKFSGFEASHGPIKAYAVILTTGEAGHPSADVLKYTYEDFKKGASDTYVTYLIRTEEKGRSQSLSEVLKYEIDVGNESTTLGYYNGKLEPLGSYRCLLRACVAGFTNITFHPQNKGLIDGAESYVSFSRYSDAVSLPQDPGVICGAVFGCIFGALVIVTVGGFIFWRKKRKDAKNNEVSFSQIKSLKRSKLIRVENFEAYFKKQQADSNCGFAEEYEDLKLVGISQPKYAAELAENRGKNRYNNVLPYDISRVKLSVQTHSTDDYINANYMPGYHSKKDFIATQGPLPNTLKDFWRMVWEKNVYAIIMLTKCVEQGRTKCEEYWPSKQAQDYGDITVAMTSEIVLPEWTIRDFTVKNIQTSESHPLRQFHFTSWPDHGVPDTTDLLINFRYLVRDYMKQSPPESPILVHCSAGVGRTGTFIAIDRLIYQIENENTVDVYGIVYDLRMHRPLMVQTEDQYVFLNQCVLDIVRSQKDSKVDLIYQNTTAMTIYENLAPVTTFGKTNGYIA

SEQ ID NO: 401 - protein, 858 aa, Homo sapiens
MLQTKDLIWTLFFLGTAVSLQVDIVPSQGEISVGESKFFLCQVAGDAKDKDISWFSPNGEKLTPNQQRISVVWNDDSSSTLTIYNANIDDAGIYKCVVTGEDGSESEATVNVKIFQKLMFKNAPTPQEFREGEDAVIVCDVVSSLPPTIIWKHKGRDVILKKDVRFIVLSNNYLQIRGIKKTDEGTYRCEGRILARGEINFKDIQVIVNVPPTIQARQNIVNATANLGQSVTLVCDAEGFPEPTMSWTKDGEQIEQEEDDEKYIFSDDSSQLTIKKVDKNDEAEYICIAENKAGEQDATIHLKVFAKPKITYVENQTAMELEEQVTLTCEASGDPIPSITWRTSTRNISSEEKASWTRPEKQETLDGHMVVRSHARVSSLTLKSIQYTDAGEYICTASNTIGQDSQSMYLEVQYAPKLQGPVAVYTWEGNQVNITCEVFAYPSATISWFRDGQLLPSSNYSNIKIYNTPSASYLEVTPDSENDFGNYNCTAVNRIGQESLEFILVQADTPSSPSIDQVEPYSSTAQVQFDEPEATGGVPILKYKAEWRAVGEEVWHSKWYDAKEASMEGIVTIVGLKPETTYAVRLAALNGKGLGEISAASEFKTQPVQGEPSAPKLEGQMGEDGNSIKVNLIKQDDGGSPIRHYLVRYRALSSEWKPEIRLPSGSDHVMLKSLDWNAEYEVYVVAENQQGKSKAAHFVFRTSAQPTAIPANGSPTSGLSTGAIVGILIVIFVLLLVVVDITCYFLNKCGLFMCIAVNLCGKAGPGAKGKDMEEGKAAFSKDESKEPIVEVRTEEERTPNHDGGKHTEPNETTPLTEPEKGPVEAKPECQETETKPAPAEVKTVPNDATQTKENESKA

SEQ ID NO: 402 - protein, 725 aa, Homo sapiens
MLQTKDLIWTLFFLGTAVSLQVDIVPSQGEISVGESKFFLCQVAGDAKDKDISWFSPNGEKLTPNQQRISVVWNDDSSSTLTIYNANIDDAGIYKCVVTGEDGSESEATVNVKIFQKLMFKNAPTPQEFREGEDAVIVCDVVSSLPPTIIWKHKGRDVILKKDVRFIVLSNNYLQIRGIKKTDEGTYRCEGRILARGEINFKDIQVIVNVPPTIQARQNIVNATANLGQSVTLVCDAEGFPEPTMSWTKDGEQIEQEEDDEKYIFSDDSSQLTIKKVDKNDEAEYICIAENKAGEQDATIHLKVFAKPKITYVENQTAMELEEQVTLTCEASGDPIPSITWRTSTRNISSEEKTLDGHMVVRSHARVSSLTLKSIQYTDAGEYICTASNTIGQDSQSMYLEVQYAPKLQGPVAVYTWEGNQVNITCEVFAYPSATISWFRDGQLLPSSNYSNIKIYNTPSASYLEVTPDSENDFGNYNCTAVNRIGQESLEFILVQADTPSSPSIDQVEPYSSTAQVQFDEPEATGGVPILKYKAEWRAVGEEVWHSKWYDAKEASMEGIVTIVGLKPETTYAVRLAALNGKGLGEISAASEFKTQPVREPSAPKLEGQMGEDGNSIKVNLIKQDDGGSPIRHYLVRYRALSSEWKPEIRLPSGSDHVMLKSLDWNAEYEVYVVAENQQGKSKAAHFVFRTSAQPTAIPATLGGNSASYTFVSLLFSAVTLLLLC

SEQ ID NO: 403 - protein, 84 aa, Homo sapiens
XQDDGGSPIRHYLVRYRALSSEWKPEIRLPSGSDHVMLKSLDWNAEYEVYVVAENQQGKSKAAHFVFRTSAQPTAIPVTLLLLC

SEQ ID NO: 404 - protein, 305 aa, Homo sapiens
YNTPSASYLEVTPDSENDFGNYNCTAVNRIGQESLEFILVQADTPSSPSIDQVEPYSSTAQVQFDEPEATGGVPILKYKAEWRAVGEEVWHSKWYDAKEASMEGIVTIVGLKPETTYAVRLAALNGKGLGEISAASEFKTQPVHSPPPPASASSSTPVPLSPPDTTWPLPALATTEPAREPSAPKLEGQMGEDGNSIKVNLIKQDDGGSPIRHYLVRYRALSSEWKPEIRLPSGSDHVMLKSLDWNAEYEVYVVAENQQGKSKAAHFVFRTSAQPTAIPATLGGNSASYTFVSLLFSAVTLLLLC

SEQ ID NO: 405 - protein, 468 aa, Homo sapiens
XSGSDHVMLKSLDWNAEYEVYVVAENQQGKSKAAHFVFRTSAQPTAIPANGSPTSGLSTGAIVGILIVIFVLLLVVVDITCYFLNKCGLFMCIAVNLCGKAGPGAKGKDMEEGKAAFSKDESKEPIVEVRTEEERTPNHDGGKHTEPNETTPLTEPELPADTTATVEDMLPSVTTVTTNSDTITETFATAQNSPTSETTTLTSSIAPPATATPDSNSVPAGQATPSKGPSASAPSPAPASAPKVAPLVDLSDTPTSTPAASNLSSSVLANQGAVLSPSAPAGVGEASKAPPASKPTPAPVPTPTGAASPLAAAAAPATEAPQAKQEAPSTKGPDPEPTQPGAAKSPAEAATALASPKSEAASVSTTNPSQGEDFKMDEGNFKTPDIDLAKDVFAALGSPAPAAGASGQAPELAPSTADSSVSPAPAKTEKGPVEAKPECQETETKPAPAEVKTVPNDATQTKENESKA

SEQ ID NO: 406 - protein, 372 aa, Homo sapiens
XTPSSPSIDQVEPYSSTAQVQFDEPEATGGVPILKYKAEWRAVGEEVWHSKWYDAKEASMEGIVTIVGLKPETTYAVRLAALNGKGLGEISAASEFKTQPVHSPPPPASASSSTPVPLSPPDKGEPSAPKLEGQMGEDGNSIKVNLIKQDDGGSPIRHYLVRYRALSSEWKPEIRLPSGSDHVMLKSLDWNAEYEVYVVAENQQGKSKAAHFVFRTSAQPTAIPANGSPTSGLSTGAIVGILIVIFVLLLVVVDITCYFLNKCGLFMCIAVNLCGKAGPGAKGKDMEEGKAAFSKDESKEPIVEVRTEEERTPNHDGGKHTEPNETTPLTEPEKGPVEAKPECQETETKPAPAEVKTVPNDATQTKENESKA

SEQ ID NO: 407 - protein, 364 aa, Homo sapiens
MLQTKDLIWTLFFLGTAVSLQVDIVPSQGEISVGESKFFLCQVAGDAKDKDISWFSPNGEKLTPNQQRISVVWNDDSSSTLTIYNANIDDAGIYKCVVTGEDGSESEATVNVKIFQKLMFKNAPTPQEFREGEDAVIVCDVVSSLPPTIIWKHKGRDVILKKDVRFIVLSNNYLQIRGIKKTDEGTYRCEGRILARGEINFKDIQVIVNVPPTIQARQNIVNATANLGQSVTLVCDAEGFPEPTMSWTKDGEQIEQEEDDEKYIFSDDSSQLTIKKVDKNDEAEYICIAENKAGEQDATIHLKVFAKPKITYVENQTAMELEEQVTLTCEASGDPIPSITWRTSTRNISSEEKASWTRPEKQEV

SEQ ID NO: 408 - protein, 848 aa, Homo sapiens
MLQTKDLIWTLFFLGTAVSLQVDIVPSQGEISVGESKFFLCQVAGDAKDKDISWFSPNGEKLTPNQQRISVVWNDDSSSTLTIYNANIDDAGIYKCVVTGEDGSESEATVNVKIFQKLMFKNAPTPQEFREGEDAVIVCDVVSSLPPTIIWKHKGRDVILKKDVRFIVLSNNYLQIRGIKKTDEGTYRCEGRILARGEINFKDIQVIVNVPPTIQARQNIVNATANLGQSVTLVCDAEGFPEPTMSWTKDGEQIEQEEDDEKYIFSDDSSQLTIKKVDKNDEAEYICIAENKAGEQDATIHLKVFAKPKITYVENQTAMELEEQVTLTCEASGDPIPSITWRTSTRNISSEEKTLDGHMVVRSHARVSSLTLKSIQYTDAGEYICTASNTIGQDSQSMYLEVQYAPKLQGPVAVYTWEGNQVNITCEVFAYPSATISWFRDGQLLPSSNYSNIKIYNTPSASYLEVTPDSENDFGNYNCTAVNRIGQESLEFILVQADTPSSPSIDQVEPYSSTAQVQFDEPEATGGVPILKYKAEWRAVGEEVWHSKWYDAKEASMEGIVTIVGLKPETTYAVRLAALNGKGLGEISAASEFKTQPVQGEPSAPKLEGQMGEDGNSIKVNLIKQDDGGSPIRHYLVRYRALSSEWKPEIRLPSGSDHVMLKSLDWNAEYEVYVVAENQQGKSKAAHFVFRTSAQPTAIPANGSPTSGLSTGAIVGILIVIFVLLLVVVDITCYFLNKCGLFMCIAVNLCGKAGPGAKGKDMEEGKAAFSKDESKEPIVEVRTEEERTPNHDGGKHTEPNETTPLTEPEKGPVEAKPECQETETKPAPAEVKTVPNDATQTKENESKA

SEQ ID NO: 409 - protein, 165 aa, Homo sapiens
EFILVQADTPSSPSIDQVEPYSSTAQVQFDEPEATGGVPILKYKAEWRAVGEEVWHSKWYDAKEASMEGIVTIVGLKPETTYAVRLAALNGKGLGEISAASEFKTQPVHSPPPPASASSSTPVPLSPPDTTWPLPALATTEPAKNIAQNHCCNMFQAGLHNALMK

SEQ ID NO: 410 - protein, 357 aa, Homo sapiens
XFILVQADTPSSPSIDQVEPYSSTAQVQFDEPEATGGVPILKYKAEWRAVGEEVWHSKWYDAKEASMEGIVTIVGLKPETTYAVRLAALNGKGLGEISAASEFKTQPVREPSAPKLEGQMGEDGNSIKVNLIKQDDGGSPIRHYLVRYRALSSEWKPEIRLPSGSDHVMLKSLDWNAEYEVYVVAENQQGKSKAAHFVFRTSAQPTAIPANGSPTSGLSTGAIVGILIVIFVLLLVVVDITCYFLNKCGLFMCIAVNLCGKAGPGAKGKDMEEGKAAFSKDESKEPIVEVRTEEERTPNHDGGKHTEPNETTPLTEPEKGPVEAKPECQETETKPAPAEVKTVPNDATQTKENESKA

SEQ ID NO: 411 - protein, 133 aa, Homo sapiens
MLQTKDLIWTLFFLGTAAKPKITYVENQTAMELEEQVTLTCEASGDPIPSITWRTSTRNISSEEKTLDGHMVVRSHARVSSLTLKSIQYTDAGEYICTASNTIGQDSQSMYLEVQYAPKLQGPVAVYTWEGNQ

SEQ ID NO: 412 - protein, 858 aa, Homo sapiens [sequence omitted]

SEQ ID NO: 413 - protein, 884 aa, Homo sapiens [sequence omitted]

SEQ ID NO: 414 - protein, 848 aa, Homo sapiens [sequence omitted]

SEQ ID NO: 415 - protein, 884 aa, Homo sapiens
MLQTKDLIWTLFFLGTAVSLQVDIVPSQGEISVGESKFFLCQVAGDAKDKDISWFSPNGEKLTPNQQRISVVWNDDSSSTLTIYNANIDDAGIYKCVVTGEDGSESEATVNVKIFQKLMFKNAPTPQEFREGEDAVIVCDVVSSLPPTIIWKHKGRDVILKKDVRFIVLSNNYLQIRGIKKTDEGTYRCEGRILARGEINFKDIQVIVNVPPTIQARQNIVNATANLGQSVTLVCDAEGFPEPTMSWTKDGEQIEQEEDDEKYIFSDDSSQLTIKKVDKNDEAEYICIAENKAGEQDATIHLKVFAKPKITYVENQTAMELEEQVTLTCEASGDPIPSITWRTSTRNISSEEKASWTRPEKQEVHAPWNWQVGRQKGQAGSAGFPGSHETLDGHMVVRSHARVSSLTLKSIQYTDAGEYICTASNTIGQDSQSMYLEVQYAPKLQGPVAVYTWEGNQVNITCEVFAYPSATISWFRDGQLLPSSNYSNIKIYNTPSASYLEVTPDSENDFGNYNCTAVNRIGQESLEFILVQADTPSSPSIDQVEPYSSTAQVQFDEPEATGGVPILKYKAEWRAVGEEVWHSKWYDAKEASMEGIVTIVGLKPETTYAVRLAALNGKGLGEISAASEFKTQPVQGEPSAPKLEGQMGEDGNSIKVNLIKQDDGGSPIRHYLVRYRALSSEWKPEIRLPSGSDHVMLKSLDWNAEYEVYVVAENQQGKSKAAHFVFRTSAQPTAIPANGSPTSGLSTGAIVGILIVIFVLLLVVVDITCYFLNKCGLFMCIAVNLCGKAGPGAKGKDMEEGKAAFSKDESKEPIVEVRTEEERTPNHDGGKHTEPNETTPLTEPEKGPVEAKPECQETETKPAPAEVKTVPNDATQTKENESKA

SEQ ID NO: 416 - protein, 136 aa, Homo sapiens [sequence omitted]

SEQ ID NO: 417 - protein, 726 aa, Homo sapiens
MLQTKDLIWTLFFLGTAVSLQVDIVPSQGEISVGESKFFLCQVAGDAKDKDISWFSPNGEKLTPNQQRISVVWNDDSSSTLTIYNANIDDAGIYKCVVTGEDGSESEATVNVKIFQKLMFKNAPTPQEFREGEDAVIVCDVVSSLPPTIIWKHKGRDVILKKDVRFIVLSNNYLQIRGIKKTDEGTYRCEGRILARGEINFKDIQVIVNVPPTIQARQNIVNATANLGQSVTLVCDAEGFPEPTMSWTKDGEQIEQEEDDEKYIFSDDSSQLTIKKVDKNDEAEYICIAENKAGEQDATIHLKVFAKPKITYVENQTAMELEEQVTLTCEASGDPIPSITWRTSTRNISSEEKTLDGHMVVRSHARVSSLTLKSIQYTDAGEYICTASNTIGQDSQSMYLEVQYAPKLQGPVAVYTWEGNQVNITCEVFAYPSATISWFRDGQLLPSSNYSNIKIYNTPSASYLEVTPDSENDFGNYNCTAVNRIGQESLEFILVQADTPSSPSIDQVEPYSSTAQVQFDEPEATGGVPILKYKAEWRAVGEEVWHSKWYDAKEASMEGIVTIVGLKPETTYAVRLAALNGKGLGEISAASEFKTQPVQGEPSAPKLEGQMGEDGNSIKVNLIKQDDGGSPIRHYLVRYRALSSEWKPEIRLPSGSDHVMLKSLDWNAEYEVYVVAENQQGKSKAAHFVFRTSAQPTAIPATLGGNSASYTFVSLLFSAVTLLLLC

SEQ ID NO: 418 - protein, 761 aa, Homo sapiens [sequence omitted]

SEQ ID NO: 419 - protein, 761 aa, Homo sapiens
MLQTKDLIWTLFFLGTAVSLQVDIVPSQGEISVGESKFFLCQVAGDAKDKDISWFSPNGEKLTPNQQRISVVWNDDSSSTLTIYNANIDDAGIYKCVVTGEDGSESEATVNVKIFQKLMFKNAPTPQEFREGEDAVIVCDVVSSLPPTIIWKHKGRDVILKKDVRFIVLSNNYLQIRGIKKTDEGTYRCEGRILARGEINFKDIQVIVNVPPTIQARQNIVNATANLGQSVTLVCDAEGFPEPTMSWTKDGEQIEQEEDDEKYIFSDDSSQLTIKKVDKNDEAEYICIAENKAGEQDATIHLKVFAKPKITYVENQTAMELEEQVTLTCEASGDPIPSITWRTSTRNISSEEKTLDGHMVVRSHARVSSLTLKSIQYTDAGEYICTASNTIGQDSQSMYLEVQYAPKLQGPVAVYTWEGNQVNITCEVFAYPSATISWFRDGQLLPSSNYSNIKIYNTPSASYLEVTPDSENDFGNYNCTAVNRIGQESLEFILVQADTPSSPSIDQVEPYSSTAQVQFDEPEATGGVPILKYKAEWRAVGEEVWHSKWYDAKEASMEGIVTIVGLKPETTYAVRLAALNGKGLGEISAASEFKTQPVHSPPPPASASSSTPVPLSPPDTTWPLPALATTEPAKGEPSAPKLEGQMGEDGNSIKVNLIKQDDGGSPIRHYLVRYRALSSEWKPEIRLPSGSDHVMLKSLDWNAEYEVYVVAENQQGKSKAAHFVFRTSAQPTAIPATLGGNSASYTFVSLLFSAVTLLLLC

SEQ ID NO: 420 - protein, 88 aa, Homo sapiens
MSDILRELLCVSEKAANIARACRQQEALFQLLIEEKKEGEKNKKFAVDFKTLADVLVQEVIKQNMENKFPGLEKNIFGEESNEFTNDW

SEQ ID NO: 421 - protein, 190 aa, Homo sapiens
MSDILRELLCVSEKAANIARACRQQEALFQLLIEEKKEGEKNKKFAVDFKTLADVLVQEVIKQNMENKFPGLEKNIFGEESNEFTNDWGEKITLRLCSTEEETAELLSKVLNGNKVASEALARVVHQDVAFTDPTLDSTEINVPQDILGIWVDPIDSTYQYIKGSADIKSNQGIFPCGLQCVTILIGVYD

SEQ ID NO: 422 - protein, 30 aa, Homo sapiens
MSDILRELLCVSEKAANIARACRQQEALFQ

SEQ ID NO: 423 - protein, 68 aa, Homo sapiens
MSDILRELLCVSEKAANIARACRQQEALFQLLIEEKKEGEKNKKFAVDFKTLADVLVQEVIKQNMENK

SEQ ID NO: 424 - protein, 831 aa, Homo sapiens
MSKKGRSKGEKPEMETDAVQMANEELRAKLTSIQIEFQQEKSKVGKLRERLQEAKLEREQEQRRHTAYISELKAKLHEEKTKELQALREGLIRQHEQEAARTAKIKEGELQRLQATLNVLRDGAADKVKTALLTEAREEARRAFDGERLRLQQEILELKAARKQAEEALSNCMQADKTKAADLRAAYQAHQDEVHRIKRECERDIRRLMDEIKGKDRVILALEKELGVQAGQTQKLLLQKEALDEQLVQVKEAERHHSSPKRELPPGIGDMVELMGVQDQHMDERDVRRFQLKIAELNSVIRKLEDRNTLLADERNELLKRSRETEVQLKPLVEKNKRMNKKNEDLLQSIQRMEEKIKNLTRENVEMKEKLSAQASLKRHTSLNDLSLTRDEQEIEFLRLQVLEQQHVIDDLSLERERLLRSKRHRGKSLKPPKKHVVETFFGFDEESVDSETLSETSYNTDRTDRTPATPEEDLDDATAREEADLRFCQLTREYQALQRAYALLQEQVGGTLDAEREARTREQLQADLLRCQAKIEDLEKLLVEKGQDSKWVEEKQLLIRTNQDLLEKIYRLEMEENQLKNEMQDAKDQNELLEFRVLELEERERRSPAFNLQITTFPENHSSALQLFCHQEGVKDVNVSELMKKLDILGDNGNLRNEEQVAIIQAGTVLALCEKWLKQIEGTEAALTQKMLDLEKEKDLFSRQKGYLEEELDYRKQALDQAYLKIQDLEATLYTALQQEPGRRAGEALSEGQREDLQAAVEKVRRQILRQSREFDSQILRERMELLQQAQQRIRELEDKLEFQKRHLKELEEKFLFLFLFFSLAFILWP

SEQ ID NO: 425 - protein, 646 aa, Homo sapiens
MSKKGRSKGEKPEMETDAVQMANEELRAKLTSIQIEFQQEKSKMDEIKGKDRVILALEKELGVQAGQTQKLLLQKEALDEQLVQVKEAERHHSSPKRELPPGIGDMVELMGVQDQHMDERDVRRFQLKIAELNSVIRKLEDRNTLLADERNELLKRSRETEVQLKPLVEKNKRMNKKNEDLLQSIQRMEEKIKNLTRENVEMKEKLSAQASLKRHTSLNDLSLTRDEQEIEFLRLQVLEQQHVIDDLSLKHVVETFFGFDEESVDSETLSETSYNTDRTDRTPATPEEDLDDATAREEADLRFCQLTREYQALQRAYALLQEQVGGTLDAEREARTREQLQADLLRCQAKIEDLEKLLVEKGQDSKWVEEKQLLIRTNQDLLEKIYRLEMEENQLKNEMQDAKDQNELLEFRVLELEERERRSPAFNLQITTFPENHSSALQLFCHQEGVKDVNVSELMKKLDILGDNGNLRNEEQVAIIQAGTVLALCEKWLKQIEGTEAALTQKMLDLEKEKDLFSRQKGYLEEELDYRKQALDQAYLKIQDLEATLYTALQQEPGRRAGEALSEGQREDLQAAVEKVRRQILRQSREFDSQILRERMELLQQAQQRIRELEDKLEFQKRHLKELEEKFLFLFLFFSLAFILWP

SEQ ID NO: 426 - protein, 66 aa, Homo sapiens
MAAIRKKLVIVGDGACGKTCLLIVFSKDQFPEVYVPTVFENYIADIEVDGKQTGSVTWAESLCLWV

SEQ ID NO: 427 - protein, 66 aa, Homo sapiens; identical to SEQ ID NO: 426

SEQ ID NO: 428 - protein, 66 aa, Homo sapiens; identical to SEQ ID NO: 426

SEQ ID NO: 429 - protein, 500 aa, Homo sapiens
MPPAVGGPVGYTPPDGGWGWAVVIGAFISIGFSYAFPKSITVFFKEIEGIFHATTSEVSWISSIMLAVMYGGGPISSILVNKYGSRIVMIVGGCLSGCGLIAASFCNTVQQLYVCIGVIGGLGLAFNLNPALTMIGKYFYKRRPLANGLAMAGSPVFLCTLAPLNQVFFGIFGWRGSFLILGGLLLNCCVAGALMRPIGPKPTKAGKDKSKASLEKAGKSGVKKDLHDANTDLIGRHPKQEKRSVFQTINQFLDLTLFTHRGFLLYLSGNVIMFFGLFAPLVFLSSYGKSQHYSSEKSAFLLSILAFVDMVARPSMGLVANTKPIRPRIQYFFAASVVANGVCHMLAPLSTTYVGFCVYAGFFGFAFGWLSSVLFETLMDLVGPQRFSSAVGLVTIVECCPVLLGPPLLGRLNDMYGDYKYTYWACGVVLIISGIYLFIGMGINYRLLAKEQKANEQKKESKEEETSIDVAGKPNEVTKAAESPDQKDTDGGPKEEESPV

SEQ ID NO: 430 - protein, 215 aa, Homo sapiens
MPPAVGGPVGYTPPDGGWGWAVVIGAFISIGFSYAFPKSITVFFKEIEGIFHATTSEVSWISSIMLAVMYGGGPISSILVNKYGSRIVMIVGGCLSGCGLIAASFCNTVQQLYVCIGVIGGLGLAFNLNPALTMIGKYFYKRRPLANGLAMAGSPVFLCTLAPLNQVFFGIFGWRGSFLILGGLLLNCCVAGALMRPIGPKPTKAGKDKSKASLE

SEQ ID NO: 431 - protein, 296 aa, Homo sapiens
MPPAVGGPVGYTPPDGGWGWAVVIGAFISIGFSYAFPKSITVFFKEIEGIFHATTSEVSWISSIMLAVMYGGGPISSILVNKYGSRIVMIVGGCLSGCGLIAASFCNTVQQLYVCIGVIGGLGLAFNLNPALTMIGKYFYKRRPLANGLAMAGSPVFLCTLAPLNQVFFGIFGWRGSFLILGGLLLNCCVAGALMRPIGPKPTKAGKDKSKASLEKAGKSGVKKDLHDANTDLIGRHPKQEKRSVFQTINQFLDLTLFTHRGFLLYLSGNVIMFFGLFAPLVFLSSYGKSQHYSSE

SEQ ID NO: 432 - protein, 456 aa, Homo sapiens
MPPAVGGPVGYTPPDGGWGWAVVIGAFISIGFSYAFPKSITVFFKEIEGIFHATTSEVSWISSIMLAVMYGGGPISSILVNKYGSRIVMIVGGCLSGCGLIAASFCNTVQQLYVCIGVIGGLGLAFNLNPALTMIGKYFYKRRPLANGLAMAGSPVFLCTLAPLNQVFFGIFGWRGSFLILGGLLLNCCVAGALMRPIGPKPTKAGKDKSKASLEKAGKSGVKKDLHDANTDLIGRHPKQEKRSVFQTINQFLDLTLFTHRGFLLYLSGNVIMFFGLFAPLVFLSSYGKSQHYSSEKSAFLLSILAFVDMVARPSMGLVANTKPIRPRIQYFFAASVVANGVCHMLAPLSTTYVGFCVYAGFFGFAFGWLSSVLFETLMDLVGPQRFSSAVGLVTIVECCPVLLGPPLLGRLNDMYGDYKYTYWACGVVLIISGIYLFIGMGINYRLLAKEQKANE

SEQ ID NO: 433 - protein, 500 aa, Homo sapiens; identical to SEQ ID NO: 429

SEQ ID NO: 434 - protein, 109 aa, Homo sapiens
MKFISTSLLLMLLVSSLSPVQGVLEVYYTSLRCRCVQESSVFIPRRFIDRIQILPRGNGCPRKEIIVWKKNKSIVCVDPQAEWIQRMMEVLRKRSSSTLPVPVFKRKIP

SEQ ID NO: 435 - protein, 729 aa, Homo sapiens
MDDLTLLDLLECPVCFEKLDVTAKVLPCQHTFCKPCLQRVFKAHKELRCPECRTPVFSNIEALPANLLLVRLLDGVRSGQSSGRGGSFRRPGTMTLQDGRKSRTNPRRLQASPFRLVPNVRIHMDGVPRAKALCNYRGQNPGDLRFNKGDIILLRRQLDENWYQGEINGISGNFPASSVEVIKQLPQPPPLCRALYNFDLRGKDKSENQDCLTFLKDDIITVISRVDENWAEGKLGDKVGIFPILFVEPNLTARHLLEKNKGRQSSRTKNLSLVSSSSRGNTSTLRRGPGSRRKVPGQFSITTALNTLNRMVHSPSGRHMVEISTPVLISSSNPSVITQPMEKADVPSSCVGQVSTYHPAPVSPGHSTAVVSLPGSQQHLSANMFVALHSYSAHGPDELDLQKGEGVRVLGKCQDGWLRGVSLVTGRVGIFPNNYVIPIFRKTSSFPDSRSPGLYTTWTLSTSSVSSQGSISEGDPRQSRPFKSVFVPTAIVNPVRSTAGPGTLGQGSLRKGRSSMRKNGSLQRPLQSGIPTLVVGSLRRSPTMVLRPQQFQFYQPQGIPSSPSAVVVEMGSKPALTGEPALTCISRGSEAWIHSAASSLIMEDKEIPIKSEPLPKPPASAPPSILVKPENSRNGIEKQVKTVRFQNYSPPPTKHYTSHPTSGKPEQPATLKASQPEAASLGPEMTVLFAHRSGCHSGQQTDLRRKSALGKATTLVSTASGTQTVFPSK

SEQ ID NO: 436 - protein, 729 aa, Homo sapiens; identical to SEQ ID NO: 435

SEQ ID NO: 437 - protein, 398 aa, Homo sapiens
MSGSSLPSALALSLLLVSGSLLPGPGAAQNAGFVKSPMSETKLTGDAFELYCDVVGSPTPEIQWWYAEVNRAESFRQLWDGARKRRVTVNTAYGSNGVSVLRITRLTLEDSGTYECRASNDPKRNDLRQNPSITWIRAQATISVLQKPRIVTSEEVIIRDSPVLPVTLQCNLTSSSHTLTYSYWTKNGVELSATRKNASNMEYRINKPRAEDSGEYHCVYHFVSAPKANATIEVKAAPDITGHKRSENKNEGQDATMYCKSVGYPHPDWIWRKKENGMPMDIVNTSGRFFIINKENYTELNIVNLQITEDPGEYECNATNAIGSASVVTVLRVRSHLAPLWPFLGILAEIIILVVIIVVYEKRKRPDEVPDDDEPAGPMKTNSTNNHKDKNLRQRNTN

SEQ ID NO: 438 - protein, 282 aa, Homo sapiens
MSGSSLPSALALSLLLVSGSLLPGPGAAQNEPRIVTSEEVIIRDSPVLPVTLQCNLTSSSHTLTYSYWTKNGVELSATRKNASNMEYRINKPRAEDSGEYHCVYHFVSAPKANATIEVKAAPDITGHKRSENKNEGQDATMYCKSVGYPHPDWIWRKKENGMPMDIVNTSGRFFIINKENYTELNIVNLQITEDPGEYECNATNAIGSASVVTVLRVRSHLAPLWPFLGILAEIIILVVIIVVYEKRKRPDEVPDDDEPAGPMKTNSTNNHKDKNLRQRNTN

SEQ ID NO: 439 - protein, 278 aa, Homo sapiens
MSGSSLPSALALSLLLVSGSLLPGPGAAQNEPRIVTSEEVIIRDSPVLPVTLQCNLTSSSHTLTYSYWTKNGVELSATRKNASNMEYRINKPRAEDSGEYHCVYHFVSAPKANATIEVKAAPDITGHKRSENKNEGQDATMYCKSVGYPHPDWIWRKKENGMPMDIVNTSGRFFIINKENYTELNIVNLQITEDPGEYECNATNAIGSASVVTVLRVRSHLAPLWPFLGILAEIIILVVIIVVYEKRKRPDEVPDAGPMKTNSTNNHKDKNLRQRNTN

SEQ ID NO: 440 - protein, 394 aa, Homo sapiens
MSGSSLPSALALSLLLVSGSLLPGPGAAQNAGFVKSPMSETKLTGDAFELYCDVVGSPTPEIQWWYAEVNRAESFRQLWDGARKRRVTVNTAYGSNGVSVLRITRLTLEDSGTYECRASNDPKRNDLRQNPSITWIRAQATISVLQKPRIVTSEEVIIRDSPVLPVTLQCNLTSSSHTLTYSYWTKNGVELSATRKNASNMEYRINKPRAEDSGEYHCVYHFVSAPKANATIEVKAAPDITGHKRSENKNEGQDATMYCKSVGYPHPDWIWRKKENGMPMDIVNTSGRFFIINKENYTELNIVNLQITEDPGEYECNATNAIGSASVVTVLRVRSHLAPLWPFLGILAEIIILVVIIVVYEKRKRPDEVPDAGPMKTNSTNNHKDKNLRQRNTN

SEQ ID NO: 441 - protein, 184 aa, Homo sapiens
MSGSSLPSALALSLLLVSGSLLPGPGAAQNAAPDITGHKRSENKNEGQDATMYCKSVGYPHPDWIWRKKENGMPMDIVNTSGRFFIINKENYTELNIVNLQITEDPGEYECNATNAIGSASVVTVLRVRSHLAPLWPFLGILAEIIILVVIIVVYEKRKRPDEVPDDDEPAGPMKTNSTNNHKD

SEQ ID NO: 442 - protein, 518 aa, Homo sapiens
XRKWSRQFLLSFWCTVCSAHQGTIFLSLKIQFADQKQEFNKRPTKIGRRSLSRSISQSSTDSYSSAASYTDSSDDETSPRDKQQKNSKGSSDFCVKNIKQAEFGRREIEIAEQEMPALMALRKRAQGEKPLAGAKIVGCTHITAQTAVLMETLGALGAQCRWAACNIYSTLNEVAAALAESGFPVFAWKGESEDDFWWCIDRCVNVEGWQPNMILDDGGDLTHWIYKKYPNMFKKIKGIVEESVTGVHRLYQLSKAGKLCVPAMNVNDSVTKQKFDNLYCCRESILDGLKRTTDMMFGGKQVVVCGYGEVGKGCCAALKAMGSIVYVTEIDPICALQACMDGFRLVKLNEVIRQVDIVITCTGNKNVVTREHLDRMKNSCIVCNMGHSNTEIDVASLRTPELTWERVRSQVDHVIWPDGKRIVLLAEGRLLNLSCSTVPTFVLSITATTQALALIELYNAPEGRYKQDVYLLPKKMDEYVASLHLPTFDAHLTELTDEQAKYLGLNKNGPFKPNYYRY

SEQ ID NO: 443 - protein, 386 aa, Homo sapiens [sequence omitted]

SEQ ID NO: 444 - protein, 143 aa, Homo sapiens
MTVVMAVCSLPLTIRCFTQAVAPDSSSEMGDLLAFRFYAFNPILDPWVFILFRKAVFQRLKLWVCCLCLGPAHGDSQTPLSQLASGRRDPRAPSAPVGKEGSCVPLSAWGEGQVEPLPPTQQSSGSAVGTSSKAEASVACSLC

SEQ ID NO: 445 - protein, 296 aa, Homo sapiens
MADSCRNLTYVRGSVGPATSTLMFVAGVVGNGLALGILSARRPARPSAFAVLVTGLAATDLLGTSFLSPAVFVAYARNSSLLGLARGGPALCDAFAFAMTFFGLASMLILFAMAVERCLALSHPYLYAQLDGPRCARLALPAIYAFCVLFCALPLLGLGQHQQYCPGSWCFLRMRWAQPGGAAFSLAYAGLVALLVAAIFLCNGSVTLSLCRMYRQQKRHQGSLGPRPRTGEDEVDHLILLALMTVVMAVCSLPLTHVERPRQDRMRWLTPVIPALWEAEAGGSFEVRSSRPVWPT

SEQ ID NO: 446 - protein, 115 aa, Homo sapiens
MGDLLAFRFYAFNPILDPWVFILFRKAVFQRLKLWVCCLCLGPAHGDSQTPLSQLASGRRDPRAPSAPVGKEGSCVPLSAWGEGQVEPLPPTQQSSGSAVGTSSKAEASVACSLC

SEQ ID NO: 447 - protein, 174 aa, Homo sapiens
MYRQQKRHQGSLGPRPRTGEDEVDHLILLALMTVVMAVCSLPLTIRCFTQAVAPDSSSEMGDLLAFRFYAFNPILDPWVFILFRKAVFQRLKLWVCCLCLGPAHGDSQTPLSQLASGRRDPRAPSAPVGKEGSCVPLSAWGEGQVEPLPPTQQSSGSAVGTSSKAEASVACSLC

SEQ ID NO: 448 - protein, 454 aa, Homo sapiens
MGENDPPAVEAPFSFRSLFGLDDLKISPVAPDADAVAAQILSLLPLKFFPIIVIGIIALILALAIGLGIHFDCSGKYRCRSSFKCIELIARCDGVSDCKDGEDEYRCVRVGGQNAVLQVFTAASWKTMCSDDWKGHYANVACAQLGFPSYVSSDNLRVSSLEGQFREEFVSIDHLLPDDKVTALHHSVYVREGCASGHVVTLQCTACGHRRGYSSRIVGGNMSLLSQWPWQASLQFQGYHLCGGSVITPLWIITAAHCVYDLYLPKSWTIQVGLVSLLDNPAPSHLVEKIVYHSKYKPKRLGNDIALMKLAGPLTFNEMIQPVCLPNSEENFPDGKVCWTSGWGATEDGAGDASPVLNHAAVPLISNKICNHRDVYGGIISPSMLCAGYLTGGVDSCQGDSGGPLVCQERRLWKLVGATSFGIGCAEVNKPGVYTRVTSFLDWIHEQMERDLKT

SEQ ID NO: 449 - protein, 344 aa, Homo sapiens
MGENDPPAVEAPFSFRSLFGLDDLKISPVAPDADAVAAQILSLLPLKFFPIIVIGIIALILALAIGLGIHFDCSGKYRCRSSFKCIELIARCDGVSDCKDGEDEYRCVRVGGQNAVLQVFTAASWKTMCSDDWKGHYANVACAQLGFPSYVSSDNLRVSSLEGQFREEFVSIDHLLPDDKVTALHHSVYVREGCASGHVVTLQCTACGHRRGYSSRIVGGNMSLLSQWPWQASLQFQGYHLCGGSVITPLWIITAAHCVYDLYLPKSWTIQVGLVSLLDNPAPSHLVEKIVYHSKYKPKRLGNDIALMKLAGPLTFNGTSGSLCGSAALPLFQEDLQLLIEAFL

SEQ ID NO: 450 - protein, 451 aa, Homo sapiens
MGENDPPAVEAPFSFRSLFGLDDLKISPVAPDAVAAQILSLLPLKFFPIIVIGIIALILALAIGLGIHFDCSGKYRCRSSFKCIELIARCDGVSDCKDGEDEYRCVRVGGQNAVLQVFTAASWKTMCSDDWKGHYANVACAQLGFPSYVSSDNLRVSSLEGQFREEFVSIDHLLPDDKVTALHHSVYVREGCASGHVVTLQCTACGHRRGYSSRIVGGNMSLLSQWPWQASLQFQGYHLCGGSVITPLWIITAAHCVYDLYLPKSWTIQVGLVSLLDNPAPSHLVEKIVYHSKYKPKRLGNDIALMKLAGPLTFNEMIQPVCLPNSEENFPDGKVCWTSGWGATEDGGDASPVLNHAAVPLISNKICNHRDVYGGIISPSMLCAGYLTGGVDSCQGDSGGPLVCQERRLWKLVGATSFGIGCAEVNKPGVYTRVTSFLDWIHEQMERDLKT

SEQ ID NO: 451 - protein, 453 aa, Homo sapiens
MGENDPPAVEAPFSFRSLFGLDDLKISPVAPDADAVAAQILSLLPLKFFPIIVIGIIALILALAIGLGIHFDCSGKYRCRSSFKCIELIARCDGVSDCKDGEDEYRCVRVGGQNAVLQVFTAASWKTMCSDDWKGHYANVACAQLGFPSYVSSDNLRVSSLEGQFREEFVSIDHLLPDDKVTALHHSVYVREGCASGHVVTLQCTACGHRRGYSSRIVGGNMSLLSQWPWQASLQFQGYHLCGGSVITPLWIITAAHCVYDLYLPKSWTIQVGLVSLLDNPAPSHLVEKIVYHSKYKPKRLGNDIALMKLAGPLTFNEMIQPVCLPNSEENFPDGKVCWTSGWGATEDGGDASPVLNHAAVPLISNKICNHRDVYGGIISPSMLCAGYLTGGVDSCQGDSGGPLVCQERRLWKLVGATSFGIGCAEVNKPGVYTRVTSFLDWIHEQMERDLKT

SEQ ID NO: 452 - protein, 734 aa, Homo sapiens
MLLWLLLLILTPGREQSGVAPKAVLLLNPPWSTAFKGEKVALICSSISHSLAQGDTYWYHDEKLLKIKHDKIQITEPGNYQCKTRGSSLSDAVHVEFSPDWLILQALHPVFEGDNVILRCQGKDNKNTHQKVYYKDGKQLPNSYNLEKITVNSVSRDNSKYHCTAYRKFYILDIEVTSKPLNIQVQELFLHPVLRASSSTPIEGSPMTLTCETQLSPQRPDVQLQFSLFRDSQTLGLGWSRSPRLQIPAMWTEDSGSYWCEVETVTHSIKKRSLRSQIRVQRVPVSNVNLEIRPTGGQLIEGENMVLICSVAQGSGTVTFSWHKEGRVRSLGRKTQRSLLAELHVLTVKESDAGRYYCAADNVHSPILSTWIRVTVRIPVSHPVLTFRAPRAHTVVGDLLELHCESLRGSPPILYRFYHEDVTLGNSSAPSGGGASFNLSLTAEHSGNYSCDADNGLGAQHSHGVSLRVTVPVSRPVLTLRAPGAQAVVGDLLELHCESLRGSFPILYWFYHEDDTLGNISAHSGGGASFNLSLTTEHSGNYSCEADNGLGAQHSKVVTLNVTGTSRNRTGLTAAGITGLVLSILVLAAAAALLHYARARRKPGGLSATGTSSHSPSECQEPSSSRPSRIDPQEPTHSKPLAPMELEPMYSNVNPGDSNPIYSQIWSIQHTKENSANCPMMHQEHEELTVLYSELKKTHPDDSAGEASSRGRAHEEDDEENYENVPRVLLASDH

SEQ ID NO: 453 - protein, 742 aa, Homo sapiens
MLLWLLLLILTPGREQSGVAPKAVLLLNPPWSTAFKGEKVALICSSISHSLAQGDTYWYHDEKLLKIKHDKIQITEPGNYQCKTRGSSLSDAVHVEFSPDWLILQALHPVFEGDNVILRCQGKDNKNTHQKVYYKDGKQLPNSYNLEKITVNSVSRDNSKYHCTAYRKFYILDIEVTSKPLNIQVQELFLHPVLRASSSTPIEGSPMTLTCETQLSPQRPDVQLQFSLFRDSQTLGLGWSRSPRLQIPAMWTEDSGSYWCEVETVTHSIKKRSLRSQIRVQRVPVSNVNLEIRPTGGQLIEGENMVLICSVAQGSGTVTFSWHKEGRVRSLGRKTQRSLLAELHVLTVKESDAGRYYCAADNVHSPILSTWIRVTVRIPVSHPVLTFRAPRAHTVVGDLLELHCESLRGSPPILYRFYHEDVTLGNSSAPSGGGASFNLSLTAEHSGNYSCDADNGLGAQHSHGVSLRVTVPVSRPVLTLRAPGAQAVVGDLLELHCESLRGSFPILYWFYHEDDTLGNISAHSGGGASFNLSLTTEHSGNYSCEADNGLGAQHSKVVTLNVTGTSRNRTGLTAAGITGLVLSILVLAAAAALLHYARARRKPGGLSATGTSSHSPSECQEPSSSRPSRIDPQEPTHSKPLAPMELEPMYSNVNPGDSNPIYSQIWSIQHTKENSANCPMMHQEHEELTVLYSELKKTHPDDSAGEASSRGRAHEEDDEENYENILNPRKNKVQDFPCLCNT

SEQ ID NO: 454 - protein, 639 aa, Homo sapiens
MLLWLLLLILTPGREQSGVAPKAVLLLNPPWSTAFKGEKVALICSSISHSLAQGDTYWYHDEKLLKIKHDKIQITEPGNYQCKTRGSSLSDAVHVEFSPDWLILQALHPVFEGDNVILRCQGKDNKNTHQKVYYKDGKQLPNSYNLEKITVNSVSRDNSKYHCTAYRKFYILDIEVTSKPLNIQVQGVPVSNVNLEIRPTGGQLIEGENMVLICSVAQGSGTVTFSWHKEGRVRSLGRKTQRSLLAELHVLTVKESDAGRYYCAADNVHSPILSTWIRVTVRIPVSHPVLTFRAPRAHTVVGDLLELHCESLRGSPPILYRFYHEDVTLGNSSAPSGGGASFNLSLTAEHSGNYSCDADNGLGAQHSHGVSLRVTVPVSRPVLTLRAPGAQAVVGDLLELHCESLRGSFPILYWFYHEDDTLGNISAHSGGGASFNLSLTTEHSGNYSCEADNGLGAQHSKVVTLNVTGTSRNRTGLTAAGITGLVLSILVLAAAAALLHYARARRKPGGLSATGTSSHSPSECQEPSSSRPSRIDPQEPTHSKPLAPMELEPMYSNVNPGDSNPIYSQIWSIQHTKENSANCPMMHQEHEELTVLYSELKKTHPDDSAGEASSRGRAHEEDDEENYENVPRVLLASDH

SEQ ID NO: 455 - protein, 734 aa, Homo sapiens; identical to SEQ ID NO: 452

SEQ ID NO: 456 - protein, 740 aa, Homo sapiens
MLLWLLLLILTPGREQSGVAPKAVLLLNPPWSTAFKGEKVALICSSISHSLAQGDTYWYHDEKLLKIKHDKIQITEPGNYQCKTRGSSLSDAVHVEFSPDWLILQALHPVFEGDNVILRCQGKDNKNTHQKVYYKDGKQLPNSYNLEKITVNSVSRDNSKYHCTAYRKFYILDIEVTSKPLNIQVQELFLHPVLRASSSTPIEGSPMTLTCETQLSPQRPDVQLQFSLFRDSQTLGLGWSRSPRLQIPAMWTEDSGSYWCEVETVTHSIKKRSLRSQIRVQRVPVSNVNLEIRPTGGQLIEGENMVLICSVAQGSGTVTFSWHKEGRVRSLGRKTQRSLLAELHVLTVKESDAGRYYCAADNVHSPILSTWIRVTVRTLLSPSVPVSHPVLTFRAPRAHTVVGDLLELHCESLRGSPPILYRFYHEDVTLGNSSAPSGGGASFNLSLTAEHSGNYSCDADNGLGAQHSHGVSLRVTVPVSRPVLTLRAPGAQAVVGDLLELHCESLRGSFPILYWFYHEDDTLGNISAHSGGGASFNLSLTTEHSGNYSCEADNGLGAQHSKVVTLNVTGTSRNRTGLTAAGITGLVLSILVLAAAAALLHYARARRKPGGLSATGTSSHSPSECQEPSSSRPSRIDPQEPTHSKPLAPMELEPMYSNVNPGDSNPIYSQIWSIQHTKENSANCPMMHQEHEELTVLYSELKKTHPDDSAGEASSRGRAHEEDDEENYENVPRVLLASDH

SEQ ID NO: 457 - protein, 256 aa, Homo sapiens
MLLWLLLLILTPGREQSGVAPKAVLLLNPPWSTAFKGEKVALICSSISHSLAQGDTYWYHDEKLLKIKHDKIQITEPGNYQCKTRGSSLSDAVHVEFSPDWLILQALHPVFEGDNVILRCQGKDNKNTHQKVYYKDGKQLPNSYNLEKITVNSVSRDNSKYHCTAYRKFYILDIEVTSKPLNIQVQELFLHPVLRASSSTPIEGSPMTLTCETQLSPQRPDVQLQFSLFRDSQTLGLGWSRSPRLQIPAMWTEDSG

SEQ ID NO: 458 - protein, 234 aa, Homo sapiens
MAFLAGPRLLDWASSPPHLQFNKFVLTGYRPASSGSGCLRSLFYLHNELGNIYTHGSVLYHLFMCHQGGSAVYARLLALDMCGVCLVNTLGALPIIHCTLACRPWLRPAALVGYTVLSGVAGWRALTAPSTSARLRAFGWQAAARLLVFGARGVGLGSGAPGSLPCYLRMDALALLGGLVNVARLPERWGPGRFDYWGNSHQIMHLLSVGSILQLHAGVVPDLLWAAHHACPRD

SEQ ID NO: 459 - protein, 273 aa, Homo sapiens
MAFLAGPRLLDWASSPPHLQFNKFVLTGYRPASSGSGCLRSLFYLHNELGNIYTHGLALLGFLVLVPMTMPWGQLGKDGWLGGTHCVACLAPPAGSVLYHLFMCHQGGSAVYARLLALDMCGVCLVNTLGALPIIHCTLACRPWLRPAALVGYTVLSGVAGWRALTAPSTSARLRAFGWQAAARLLVFGARGVGLGSGAPGSLPCYLRMDALALLGGLVNVARLPERWGPGRFDYWGNSHQIMHLLSVGSILQLHAGVVPDLLWAAHHACPRD

SEQ ID NO: 460 - protein, 199 aa, Homo sapiens
MAFLAGPRLLDWASSPPHLQFNKFVLTGYRPASSGSGCLRSLFYLHNELGNIYTHGALPIIHCTLACRPWLRPAALVGYTVLSGVAGWRALTAPSTSARLRAFGWQAAARLLVFGARGVGLGSGAPGSLPCYLRMDALALLGGLVNVARLPERWGPGRFDYWGNSHQIMHLLSVGSILQLHAGVVPDLLWAAHHACPRD

SEQ ID NO: 461 - protein, 206 aa, Homo sapiens
MTMPWGQLGKDGWLGGTHCVACLAPPAGSVLYHLFMCHQGGSAVYARLLALDMCGVCLVNTLGALPIIHCTLACRPWLRPAALVGYTVLSGVAGWRALTAPSTSARLRAFGWQAAARLLVFGARGVGLGSGAPGSLPCYLRMDALALLGGLVNVARLPERWGPGRFDYWGNSHQIMHLLSVGSILQLHAGVVPDLLWAAHHACPRD

SEQ ID NO: 462 - protein, 206 aa, Homo sapiens; identical to SEQ ID NO: 461

SEQ ID NO: 463 - protein, 208 aa, Homo sapiens
MGAQGAQESIKAMWRVPGTTRRPVTGESPGMHRPEAMLLLLTLALLGGPTWAGKMYGPGGGKYFSTTEDYDHEITGLRVSVGLLLVKSVQVKLGDSWDVKLGALGGNTQEVTLQPGEYITKVFVAFQAFLRGMVMYTSKDRYFYFGKLDGQISSAYPSQEGQVLVGIYGQYQLLGIKSIGFEWNYPLEEPTTEPPVNLTYSANSPVGR

SEQ ID NO: 464 - protein, 95 aa, Homo sapiens
XAQGAQESIKAMWRVPGTTRRPVTGESPGMHRPEAMLLLLTLALLGGPTWAGKMYGPGGGKYFSTTEDYDHEITGLRVSVGLLLVKRFLEGVIYE

SEQ ID NO: 465 - protein, 69 aa, Homo sapiens
XRRPVTGESPGMHRPEAMLLLLTLALLGGPTWAGKMYGPGGGKYFSTTEDYDHEITGLRVSVGLLLVKR

SEQ ID NO: 466 - protein, 178 aa, Homo sapiens
MHRPEAMLLLLTLALLGGPTWAGKMYGPGGGKYFSTTEDYDHEITGLRVSVGLLLVKSVQVKLGDSWDVKLGALGGNTQEVTLQPGEYITKVFVAFQAFLRGMVMYTSKDRYFYFGKLDGQISSAYPSQEGQVLVGIYGQYQLLGIKSIGFEWNYPLEEPTTEPPVNLTYSANSPVGR

SEQ ID NO: 467 - protein, 399 aa, Homo sapiens
MAELLRSLQDSQLVARFQRRCGLFPAPDEGPRENGADPTERAARVPGVEHLPAANGKGGEAPANGLRRAAAPEAYVQKYVVKNYFYYYLFQFSAALGQEVFYITFLPFTHWNIDPYLSRRLIIIWVLVMYIGQVAKDVLKWPRPSSPPVVKLEKRLIAEYGMPSTHAMAATAIAFTLLISTMDRYQYPFVLGLVMAVVFSTLVCLSRLYTGMHTVLDVLGGVLITALLIVLTYPAWTFIDCLDSASPLFPVCVIVVPFFLCYNYPVSDYYSPTRADTTTILAAGAGVTIGFWINHFFQLVSKPAESLPVIQNIPPLTTYMLVLGLTKFAVGIVLILLVRQLVQNLSLQVLYSWFKVVTRNKEARRRLEIEVPYKFVTYTSVGICATTFVPMLHRFLGLP

SEQ ID NO: 468 - protein, 128 aa, Homo sapiens
MDVYGTTKAIELLDPTASRLPTPMPWDLSNKAVPEPKGFGPKDLALPTGNLKATWHLCLREHLPTLHTRRGVCHLLLSRCQHPPCALRPHILLQILCLAGLQRYGQVPCVPLADRGGSPCAGPSCSEG

SEQ ID NO: 469 - protein, 104 aa, Homo sapiens
MDVYGTTKAIELLDPTASRLPTPMPWDLSNKAVPEPKATPGEECAICFYHAANTRLVPCGHTYFCRYCAWRVFSDTAKCPVCRWQIEAVAPAQGPPALRVEEGS

SEQ ID NO: 470 - protein, 83 aa, Homo sapiens
MAPGCKIMKVMPSKFLCEFVLLQKDLGQLMESRTYAYLCCPPRVSGCTPTLSPRPSRLIMLQMWIPLRNLYSQLWLKALWSLA

SEQ ID NO: 471 - protein, 438 aa, Homo sapiens
MPCTCTWRNWRQWIRPLVAVIYLVSIVVAVPLCVWELQKLEVGIHTKAWFIAGIFLLLTIPISLWVILQHLVHYTQPELQKPIIRILWMVPIYSLDSWIALKYPGIAIYVDTCRECYEAYVIYNFMGFLTNYLTNRYPNLVLILEAKDQQKHFPPLCCCPPWAMGEVLLFRCKLGVLQYTVVRPFTTIVALICELLGIYDEGNFSFSNAWTYLVIINNMSQLFAMYCLLLFYKVLKEELSPIQPVGKFLCVKLVVFVSFWQAVVIALLVKVGVISEKHTWEWQTVEAVATGLQDFIICIEMFLAAIAHHYTFSYKPYVQEAEEGSCFDSFLAMWDVSDIRDDISEQVRHVGRTVRGHPRKKLFPEDQDQNEHTSLLSSSSQDAISIASSMPPSPMGHYQGFGHTVTPQTTPTTAKISDEILSDTIGEKKEPSDKSVDS

SEQ ID NO: 472 - protein, 72 aa, Homo sapiens
LGIYDEGNFSFSNAWTYLVIINNMSQLFAMYCLLLFYKVLKEELSPIQPVGKFLCVKLVVFVSFCIRGNGKL

SEQ ID NO: 473 - protein, 261 aa, Homo sapiens
MPCTCTWRNWRQWIRPLVAVIYLVSIVVAVPLCVWELQKLEVGIHTKAWFIAGIFLLLTIPISLWVILQHLVHYTQPELQKPIIRILWMVPIYSLDSWIALKYPGIAIYVDTCRECYEAYVIYNFMGFLTNYLTNRYPNLVLILEAKDQQKHFPPLCCCPPWAMGEVLLFRCKLGVLQYTVVRPFTTIVALICELLGIYDEGNFSFSNAWTYLVIINNMSQLFAMYCLLLFYKVLKEELSPIQPVGKFLCVKLVVFVSFWP

SEQ ID NO: 474 - protein, 115 aa, Homo sapiens
MLWFQGNSMQLARSSFGLFLRNCSASKTTLPVLTLFTKDPCPLCDEAKEVLKPYENRFILQEVNITLPENSVWYERYKFDIPVFHLNGQFLMMHRVNTSKLEKQLLKLEQQSTGG

SEQ ID NO: 475 - protein, 55 aa, Homo sapiens
MLWFQGNSMQLARSSFGLFLRNCSASKTTLPVLTLFTKEFLSTRSLFACCHPCKM

SEQ ID NO: 476 - protein, 59 aa, Homo sapiens
MLWFQGNSMQLARSSFGLFLRNCSASKTTLPVLTLFTKDPCPLCDEAKEVLKPYENRVI

SEQ ID NO: 477 - protein, 138 aa, Homo sapiens
MLWFQGNSMQLARSSFGLFLRNCSASKTTLPVLTLFTKDPCPLCDEAKEVLKPYENRQPYKDQKLPGTRRRRSPSSPSHPHMASQSGKRYNLTLNQVLSFDYDMGLDAPKTISSDCGAFYCLRMFKSPDMTCCFYPKQ

SEQ ID NO: 478 - protein, 57 aa, Homo sapiens
KTTLPVLTLFTKDPCPLCDEAKEVLKPYENRFLMMHRVNTSKLEKQLLKLEQQSTGG

SEQ ID NO: 479 - protein, 138 aa, Homo sapiens; identical to SEQ ID NO: 477

SEQ ID NO: 480 - protein, 89 aa, Homo sapiens
MLWFQGNSMQLARSSFGLFLRNCSASKTTLPVLTLFTKDPCPLCDEAKEVLKPYENRRSTSQSLGNAIYRKKVLHSCCCPGGLNFLMKE

SEQ ID NO: 481 - protein, 215 aa, Homo sapiens
MKDYDELLKYYELHETIGTGGFAKVKLACHILTGEMVAIKIMDKNTLGSDLPRIKTEIEALKNLRHQHICQLYHVLETANKIFMVLEENLLFDEYHKLKLIDFGLCAKPKGNKDYHLQTCCGSLAYAAPELIQGKSYLGSEADVWSMGILLYVLMCGFLPFDDDNVMALYKKIMRGKYDVPKWLSPSSILLLQQMLQVDPKKRISMKNLLNHPWI

SEQ ID NO: 482 - protein, 580 aa, Homo sapiens
MMNFSNIMNYMKLLGQYCPGGELFDYIISQDRLSEEETRVVFRQIVSAVAYVHSQGYAHRDLKPENLLFDEYHKLKLIDFGLCAKPKGNKDYHLQTCCGSLAYAAPELIQGKSYLGSEADVWSMGILLYVLMCGFLPFDDDNVMALYKKIMRGKYDVPKWLSPSSILLLQQMLQVDPKKRISMKNLLNHPWIMQDYNYPVEWQSKNPFIHLDDDCVTELSVHHRNNRQTMEDLISLWQYDHLTATYLLLLAKKARGKPVRLRLSSFSCGQASATPFTDIKSNNWSLEDVTASDKNYVAGLIDYDWCEDDLSTGAATPRTSQFTKYWTESNGVESKSLTPALCRTPANKLKNKENVYTPKSAVKNEEYFMFPEPKTPVNKNQHKREILTTPNRYTTPSKARNQCLKETPIKIPVNSTGTDKLMTGVISPERRCRSVELDLNQAHMEETPKRKGAKVFGSLERGLDKVITVLTRSKRKGSARDGPRRLKLHYNVTTTRLVNPDQLLNEIMSILPKKHVDFVQKGYTLKCQTQSDFGKVTMQFELEVCQLQKPDVVGIRRQRLKGDAWVYKRLVEDILSSCKV

SEQ ID NO: 483 - protein, 520 aa, Homo sapiens
MVLEENLLFDEYHKLKLIDFGLCAKPKGNKDYHLQTCCGSLAYAAPELIQGKSYLGSEADVWSMGILLYVLMCGFLPFDDDNVMALYKKIMRGKYDVPKWLSPSSILLLQQMLQVDPKKRISMKNLLNHPWIMQDYNYPVEWQSKNPFIHLDDDCVTELSVHHRNNRQTMEDLISLWQYDHLTATYLLLLAKKARGKPVRLRLSSFSCGQASATPFTDIKSNNWSLEDVTASDKNYVAGLIDYDWCEDDLSTGAATPRTSQFTKYWTESNGVESKSLTPALCRTPANKLKNKENVYTPKSAVKNEEYFMFPEPKTPVNKNQHKREILTTPNRYTTPSKARNQCLKETPIKIPVNSTGTDKLMTGVISPERRCRSVELDLNQAHMEETPKRKGAKVFGSLERGLDKVITVLTRSKRKGSARDGPRRLKLHYNVTTTRLVNPDQLLNEIMSILPKKHVDFVQKGYTLKCQTQSDFGKVTMQFELEVCQLQKPDVVGIRRQRLKGDAWVYKRLVEDILSSCKV

SEQ ID NO: 484 - protein, 619 aa, Homo sapiens
MMNFSNIMNYMKLLGQSDLPRIKTEIEALKNLRHQHICQLYHVLETANKIFMVLEYCPGGELFDYIISQDRLSEEETRVVFRQIVSAVAYVHSQGYAHRDLKPENLLFDEYHKLKLIDFGLCAKPKGNKDYHLQTCCGSLAYAAPELIQGKSYLGSEADVWSMGILLYVLMCGFLPFDDDNVMALYKKIMRGKYDVPKWLSPSSILLLQQMLQVDPKKRISMKNLLNHPWIMQDYNYPVEWQSKNPFIHLDDDCVTELSVHHRNNRQTMEDLISLWQYDHLTATYLLLLAKKARGKPVRLRLSSFSCGQASATPFTDIKSNNWSLEDVTASDKNYVAGLIDYDWCEDDLSTGAATPRTSQFTKYWTESNGVESKSLTPALCRTPANKLKNKENVYTPKSAVKNEEYFMFPEPKTPVNKNQHKREILTTPNRYTTPSKARNQCLKETPIKIPVNSTGTDKLMTGVISPERRCRSVELDLNQAHMEETPKRKGAKVFGSLERGLDKVITVLTRSKRKGSARDGPRRLKLHYNVTTTRLVNPDQLLNEIMSILPKKHVDFVQKGYTLKCQTQSDFGKVTMQFELEVCQLQKPDVVGIRRQRLKGDAWVYKRLVEDILSSCKV

SEQ ID NO: 485 - protein, 129 aa, Homo sapiens
MVLEYCPGGELFDYIISQDRLSEEETRVVFRQIVSAVAYVHSQGYAHRDLKPENLLFDEYHKLKLIDFGLCAKPKGNKDYHLQTCCGSLAYAAPELIQGKSYLGSEMFGAWAYCYMFLCVDFYHLMMIM

SEQ ID NO: 486 - protein, 532 aa, Homo sapiens
MAPSFTARIQLFLLRALGFLIGLVGRAALVLGGPKFASKTPRPVTEPLLLLSGMQLAKLIRQRKVKCIDVVQAYINRIKDVNPMINGIVKYRFEEAMKEAHAVDQKLAEKQEDEATLENKWPFLGVPLTVKEAFQLQGMPNSSGLMNRRDAIAKTDATVVALLKGAGAIPLGITNCSELCMWYESSNKIYGRSNNPYDLQHIVGGSSGGEGCTLAAACSVIGVGSDIGGSIRMPAFFNGIFGHKPSPGVVPNKGQFPLAVGAQELFLCTGPMCRYAEDLAPMLKVMAGPGIKRLKLDTKVHLKDLKFYWMEHDGGSFLMSKVDQDLIMTQKKVVVHLETILGASVQHVKLKKMKYSFQLWIAMMSAKGHDGKEPVKFVDLLGDHGKHVSPLWELIKWCLGLSVYTIPSIGLALLEEKLRYSNEKYQKFKAVEESLRKELVDMLGDDGVFLYPSHPTVAPKHHVPLTRPFNFAYTGVFSALGLPVTQCPLGLNAKGLPLGIQVVAGPFNDHLTLAVAQYLEKTFGGWVCPGKF

SEQ ID NO: 487 - protein, 563 aa, Homo sapiens
MGLQACLLGLFALILSGKCSYSPEPDQRRTLPPGWVSLGRADPEEELSLTFALRQQNVERLSELVQAVSDPSSPQYGKYLTLENVADLVRPSPLTLHTVQKWLLAAGAQKCHSVITQDFLTCWLSIRQAELLLPGAEFHHYVGGPTETHVVRSPHPYQLPQALAPHVDFVGGLHRFPPTSSLRQRPEPQVTGTVGLHLGVTPSVIRKRYNLTSQDVGSGTSNNSQACAQFLEQYFHDSDLAQFMRLFGGNFAHQASVARVVGQQGRGRAGIEASLDVQYLMSAGANISTWVYSSPGRHEGQEPFLQWLMLLSNESALPHVHTVSYGDDEDSLSSAYIQRVNTELMKAAARGLTLLFASGDSGAGCWSVSGRHQFRPTFPASSPYVTTVGGTSFQEPFLITNEIVDYISGGGFSNVFPRPSYQEEAVTKFLSSSPHLPPSSYFNASGRAYPDVAALSDGYWVVSNRVPIPWVSGTSASTPVFGGILSLINEHRILSGRPPLGFLNPRLYQQHGAGLFDVTRGCHESCLDEEVEGQGFCSGPGWDPVTGWGTPNFPALLKTLLNP

SEQ ID NO: 488 - protein, 177 aa, Homo sapiens
MGLQACLLGLFALILSGKCSYSPEPDQRRTLPPGWVSLGRADPEEELSLTFALRQQNVERLSELVQAVSDPSSPQYGKYLTLENVADLVRPSPLTLHTVQKWLLAAGAQKCHSVITQDFLTCWLSIRQAELLLPGAEFHHYVGGPTETHVAGMRDRSPSCSGSCCSVMSQPCHMCIL

SEQ ID NO: 489 - protein, 39 aa, Homo sapiens
MGLQACLLGLFALILSGKCSYSPEPDQRRTKIPDPRECG

SEQ ID NO: 490 - protein, 30 aa, Homo sapiens
MGLQACLLGLFALILSGKCSYSPEPDQRRT

SEQ ID NO: 491 - protein, 387 aa, Homo sapiens
MREPLEAFKLADLDFRKSSLASGWRMASGAFTMDQFPESVTENFEYDDLAEACYIGDIVVFGTVFLSIFYSVIFAIGLVGNLLVVFALTNSKKPKSVTDIYLLNLALSDLLFVATLPFWTHYLINEKGLHNAMCKFTTAFFFIGFFGSIFFITVISIDRYLAIVLAANSMNNRTVQHGVTISLGVWAAAILVAAPQFMFTKQKENECLGDYPEVLQEIWPVLRNVETNFLGFLLPLLIMSYCYFRIIQTLFSCKNHKKAKAIKLILLVVIVFFLFWTPYNVMIFLETLKLYDFFPSCDMRKDLRLALSVTETVAFSHCCLNPLIYAFAGEKFRRYLYHLYGKCLAVLCGRSVHVDFSSSESQRSRHGSVLSSNFTYHTSDGDALLLL

SEQ ID NO: 492 - protein, 355 aa, Homo sapiens
MDQFPESVTENFEYDDLAEACYIGDIVVFGTVFLSIFYSVIFAIGLVGNLLVVFALTNSKKPKSVTDIYLLNLALSDLLFVATLPFWTHYLINEKGLHNAMCKFTTAFFFIGFFGSIFFITVISIDRYLAIVLAANSMNNRTVQHGVTISLGVWAAAILVAAPQFMFTKQKENECLGDYPEVLQEIWPVLRNVETNFLGFLLPLLIMSYCYFRIIQTLFSCKNHKKAKAIKLILLVVIVFFLFWTPYNVMIFLETLKLYDFFPSCDMRKDLRLALSVTETVAFSHCCLNPLIYAFAGEKFRRYLYHLYGKCLAVLCGRSVHVDFSSSESQRSRHGSVLSSNFTYHTSDGDALLLL

SEQ ID NO: 493 - protein, 106 aa, Homo sapiens
MDQFPESVTENFEYDDLAEACYIGDIVVFGTVFLSIFYSVIFAIGLVGNLLVVFALTNSKKPKSVTDIYLLNLALSDLLFVATLPFWTHYLINEKGLHNAMCKFTT

SEQ ID NO: 494 - protein, 166 aa, Homo sapiens
MDQFPESVTENFEYDDLAEACYIGDIVVFGTVFLSIFYSVIFAIGLVGNLLVVFALTNSKKPKSVTDIYLLNLALSDLLFVATLPFWTHYLINEKGLHNAMCKFTTAFFFIGFFGSIFFITVISIDRYLAIVLAANSMNNRTVQHGVTISLGVWAAAILVAAPQFM

SEQ ID NO: 495 - protein, 355 aa, Homo sapiens; identical to SEQ ID NO: 492

SEQ ID NO: 496 - protein, 355 aa, Homo sapiens; identical to SEQ ID NO: 492

SEQ ID NO: 497 - protein, 268 aa, Homo sapiens
MSGKHYKGPEVSCCIKYFIFGFNVIFWFLGITFLGIGLWAWNEKGVLSNISSITDLGGFDPVWLFLVVGGVMFILGFAGCIGALRENTFLLKFFSVFLGIIFFLELTAGVLAFVFKDWIKDQLYFFINNNIRAYRDDIDLQNLIDFTQEYWQCCGAFGADDWNLNIYFNCTDSNASRERCGVPFSCCTKDPAEDVINTQCGYDARQKPEVDQQIVIYTKGCVPQFEKWLQDNLTIVAGIFIGIALLQIFGICLAQNLVSDIEAVRASW

SEQ ID NO: 498 - protein, 197 aa, Homo sapiens
MFILGFAGCIGALRENTFLLKFFSVFLGIIFFLELTAGVLAFVFKDWIKDQLYFFINNNIRAYRDDIDLQNLIDFTQEYWQCCGAFGADDWNLNIYFNCTDSNASRERCGVPFSCCTKDPAEDVINTQCGYDARQKPEVDQQIVIYTKGCVPQFEKWLQDNLTIVAGIFIGIALLQIFGICLAQNLVSDIEAVRASW

SEQ ID NO: 499 - protein, 193 aa, Homo sapiens
MSGKHYKGPEVSCCIKYFIFGFNVIFWFLGITFLGIGLWAWNEKGVLSNISSITDLGGFDPVWLFLVVGGVMFILGFAGCIGALRENTFLLKFFSVFLGIIFFLELTAGVLAFVFKDWIKDQLYFFINNNIRAYRDDIDLQNLIDFTQEYIPMQVESDVAFHSPAALKIPQKMSSTLSVAMMPGKNQKLTSRL

SEQ ID NO: 500 - protein, 60 aa, Homo sapiens
MFILGFAGCIGALRENTFLLKFFSVFLGIIFFLELTAGVLAFVFKDWIKDQLYFFINNNI

SEQ ID NO: 501 - protein, 38 aa, Homo sapiens
MFILGFAGCIGALRENTFLLKFLKSPVLTEQAENYTFL

SEQ ID NO: 502 - protein, 110 aa, Homo sapiens
MFILGFAGCIGALRENTFLLKFFSVFLGIIFFLELTAGVLAFVFKDWIKDQLYFFINNNIRAYRDDIDLQNLIDFTQEYWQCCGAFGADDWNLNIYFNCTDSNASRERCG

SEQ ID NO: 503 - protein, 87 aa, Homo sapiens
XVSCCIKYFIFGFNVIFWFLGITFLGIGLWAWNEKGVLSNISSITDLGGFDPVWLFLVVGGVMFILGFAGCIGALRENTFLLKFDKQ

SEQ ID NO: 504 - protein, 29 aa, Homo sapiens
SFASLKSGTCACTQTGRAGEAGGLRKAGS

SEQ ID NO: 505 - protein, 37 aa, Homo sapiens
MLLYPAGFSFASLKSGTCACTQTGRAGEAGGLRKAGS

SEQ ID NO: 506 - protein, 677 aa, Homo sapiens
MPGFLVRILPLLLVLLLLGPTRGLRNATQRMFEIDYSRDSFLKDGQPFRYISGSIHYSRVPRFYWKDRLLKMKMAGLNAIQTYVPWNFHEPWPGQYQFSEDHDVEYFLRLAHELGLLVILRPGPYICAEWEMGGLPAWLLEKESILLRSSDPDYLAAVDKWLGVLLPKMKPLLYQNGGPVITVQVENEYGSYFACDFDYLRFLQKRFRHHLGDDVVLFTTDGAHKTFLKCGALQGLYTTVDFGTGSNITDAFLSQRKCEPKGPLINSEFYTGWLDHWGQPHSTIKTEAVASSLYDILARGASVNLYMFIGGTNFAYWNGANSPYAAQPTSYDYDAPLSEAGDLTEKYFALRNIIQKFEKVPEGPIPPSTPKFAYGKVTLEKLKTVGAALDILCPSGPIKSLYPLTFIQVKQHYGFVLYRTTLPQDCSNPAPLSSPLNGVHDRAYVAVDGIPQGVLERNNVITLNITGKAGATLDLLVENMGRVNYGAYINDFKGLVSNLTLSSNILTDWTIFPLDTEDAVCSHLGGWGHRDSGHHDEAWAHNSSNYTLPAFYMGNFSIPSGIPDLPQDTFIQFPGWTKGQVWINGFNLGRYWPARGPQLTLFVPQHILMTSAPNTITVLELEWAPCSSDDPELCAVTFVDRPVIGSSVTYDHPSKPVEKRLMPPPPQKNKDSWLDHV

SEQ ID NO: 507 - protein, 546 aa, Homo sapiens
MPGFLVRILPLLLVLLLLGPTRGLRNATQRMFEIDYSRDSFLKDGQPFRYISGSIHYSRVPRFYWKDRLLKMKMAGLNAIQTLPGSCGQVVGSPSAQDEASPLSEWRASYNSAGSNITDAFLSQRKCEPKGPLINSEFYTGWLDHWGQPHSTIKTEAVASSLYDILARGASVNLYMFIGGTNFAYWNGANSPYAAQPTSYDYDAPLSEAGDLTEKYFALRNIIQKFEKVPEGPIPPSTPKFAYGKVTLEKLKTVGAALDILCPSGPIKSLYPLTFIQVKQHYGFVLYRTTLPQDCSNPAPLSSPLNGVHDRAYVAVDGIPQGVLERNNVITLNITGKAGATLDLLVENMGRVNYGAYINDFKGLVSNLTLSSNILTDWTIFPLDTEDAVCSHLGGWGHRDSGHHDEAWAHNSSNYTLPAFYMGNFSIPSGIPDLPQDTFIQFPGWTKGQVWINGFNLGRYWPARGPQLTLFVPQHILMTSAPNTITVLELEWAPCSSDDPELCAVTFVDRPVIGSSVTYDHPSKPVEKRLMPPPPQKNKDSWLDHV

SEQ ID NO: 508 - protein, 647 aa, Homo sapiens
MFEIDYSRDSFLKDGQPFRYISGSIHYSRVPRFYWKDRLLKMKMAGLNAIQTYVPWNFHEPWPGQYQFSEDHDVEYFLRLAHELGLLVILRPGPYICAEWEMGGLPAWLLEKESILLRSSDPDYLAAVDKWLGVLLPKMKPLLYQNGGPVITVQVENEYGSYFACDFDYLRFLQKRFRHHLGDDVVLFTTDGAHKTFLKCGALQGLYTTVDFGTGSNITDAFLSQRKCEPKGPLINSEFYTGWLDHWGQPHSTIKTEAVASSLYDILARGASVNLYMFIGGTNFAYWNGANSPYAAQPTSYDYDAPLSEAGDLTEKYFALRNIIQKFEKVPEGPIPPSTPKFAYGKVTLEKLKTVGAALDILCPSGPIKSLYPLTFIQVKQHYGFVLYRTTLPQDCSNPAPLSSPLNGVHDRAYVAVDGIPQGVLERNNVITLNITGKAGATLDLLVENMGRVNYGAYINDFKGLVSNLTLSSNILTDWTIFPLDTEDAVCSHLGGWGHRDSGHHDEAWAHNSSNYTLPAFYMGNFSIPSGIPDLPQDTFIQFPGWTKGQVWINGFNLGRYWPARGPQLTLFVPQHILMTSAPNTITVLELEWAPCSSDDPELCAVTFVDRPVIGSSVTYDHPSKPVEKRLMPPPPQKNKDSWLDHV

SEQ ID NO: 509 - protein, 185 aa, Homo sapiens
MPGFLVRILPLLLVLLLLGPTRGLRVENEYGSYFACDFDYLRFLQKRFRHHLGDDVVLFTTDGAHKTFLKCGALQGLYTTVDFGTGSNITDAFLSQRKCEPKGPLINSEFYTGWLDHWGQPHSTIKTEAVASSLYDILARGASVNLYMFIGGTNFAYWNGANSPYAAQPTSYDYDAPLSEAGDLT

SEQ ID NO: 510 - protein, 147 aa, Homo sapiens
MPGFLVRILPLLLVLLLLGPTRGLRSRFLPWAFHLPRQAPKSQLPLHKRGTKTAPNEHASSNRSGRRRRRQQWNATQRMFEIDYSRDSFLKDGQPFRYISGSIHYSRVPRFYWKDRLLKMKMAGLNAIQTYVPWNFHEPWPGQYQFS

SEQ ID NO: 511 - protein, 41 aa, Homo sapiens
MPGFLVRILPLLLVLLLLGPTRGLRITWQLWTSGWESFCPR

SEQ ID NO: 512 - protein, 114 aa, Homo sapiens
MGGLPAWLLEKESILLRSSDPDYLAAVDKWLGVLLPKMKPLLYQNGGPVITVQVENEYGSYFACDFDYLRFLQKRFRHHLGDDVVLFTTDGAHKTFLKCGALQGLYTTVDFGTG

SEQ ID NO: 513 - protein, 84 aa, Homo sapiens
MFEIDYSRDSFLKDGQPFRYISGSIHYSRVPRFYWKDRLLKMKMAGLNAIQTLPGSCGQVVGSPSAQDEASPLSEWRASYNSAG

SEQ ID NO: 514 - protein, 102 aa, Homo sapiens
MFEIDYSRDSFLKDGQPFRYISGSIHYSRVPRFYWKDRLLKMKMAGLNAIQTYVPWNFHEPWPGQYQFSEDHDVEYFLRLAHELGLLVILRPGPYICAEWEM

SEQ ID NO: 515 - protein, 150 aa, Homo sapiens
XLGWSKAGSGSVCLALDQLRDVIESQEELIHQLRNVMVLQDENFVSKEEFQAVEKKLVEEKAAHAKTKVLLAKEEEKLQFALGEVEVLSKQLEKEKLAFEKALSSVKSKVLQESSKKDQLITKCNGITCLTSGSRSSRRATWPRCWTRSI

SEQ ID NO: 516 - protein, 63 aa, Homo sapiens
MAASGEPQRQWQEEVAAVVVVGSCMTDLVRRMCMRICYVHFCCQPTPQLQNCSSLCVITCQEN

SEQ ID NO: 517 - protein, 518 aa, Homo sapiens
MALERLCSVLKVLLITVLVVEGIAVAQKTQDGQNIGIKHIPATQCGIWVRTSNGGHFASPNYPDSYPPNKECIYILEAAPRQRIELTFDEHYYIEPSFECRFDHLEVRDGPFGFSPLIDRYCGVKSPPLIRSTGRFMWIKFSSDEELEGLGFRAKYSFIPDPDFTYLGDCQFELSGADGIVRSSQVEQEEKTKPGQAVDCIWTIKATPKAKIYLRFLDYQMEHSNECKRNFVAVYDGSSSIENLKAKFCSTVANDVMLKTGIGVIRMWADEGSRLSRFRMLFTSFVEPPCTSSTFFCHSNMCINNSLVCNGVQNCAYPWDENHCKEKKKAGVFEQITKTHGTIIGITSGIVLVLLIISILVQVKQPRKKVMACKTAFNKTGFQEVFDPPHYELFSLRDKEISADLADLSEELDNYQKMRRSSTASRCIHDHHCGSQASSVKQSRTNLSSMELPFRNDFAQPQPMKTFNSTFKKSSYTFKQGHECPEQALEDRVMEEIPCEIYVRGREDSAQASISIDF

SEQ ID NO: 518 - protein, 525 aa, Homo sapiens
MALERLCSVLKVLLITVLVVEGIAVAQKTQDGQNIGIKHIPATQCGIWVRTSNGGHFASPNYPDSYPPNKECIYILEAAPRQRIELTFDEHYYIEPSFECRFDHLEVRDGPFGFSPLIDRYCGVKSPPLIRSTGRFMWIKFSSDEELEGLGFRAKYSFIPDPDFTYLGGILNPIPDCQFELSGADGIVRSSQVEQEEKTKPGQAVDCIWTIKATPKAKIYLRFLDYQMEHSNECKRNFVAVYDGSSSIENLKAKFCSTVANDVMLKTGIGVIRMWADEGSRLSRFRMLFTSFVEPPCTSSTFFCHSNMCINNSLVCNGVQNCAYPWDENHCKEKKKAGVFEQITKTHGTIIGITSGIVLVLLIISILVQVKQPRKKVMACKTAFNKTGFQEVFDPPHYELFSLRDKEISADLADLSEELDNYQKMRRSSTASRCIHDHHCGSQASSVKQSRTNLSSMELPFRNDFAQPQPMKTFNSTFKKSSYTFKQGHECPEQALEDRVMEEIPCEIYVRGREDSAQASISIDF

SEQ ID NO: 519 - protein, 365 aa, Homo sapiens
XLIDRYCGVKSPPLIRSTGRFMWIKFSSDEELEGLGFRAKYSFIPDPDFTYLGDCQFELSGADGIVRSSQVEQEEKTKPGQAVDCIWTIKATPKAKIYLRFLDYQMEHSNECKRNFVAVYDGSSSIENLKAKFCSTVANDVMLKTGIGVIRMWADEGSRLSRFRMLFTSFVEQKKKAGVFEQITKTHGTIIGITSGIVLVLLIISILVQVKQPRKKVMACKTAFNKTGFQEVFDPPHYELFSLRDKEISADLADLSEELDNYQKMRRSSTASRCIHDHHCGSQASSVKQSRTNLSSMELPFRNDFAQPQPMKTFNSTFKKSSYTFKQGHECPEQALEDRVMEEIPCEIYVRGREDSAQASISIDF

SEQ ID NO: 520 - protein, 194 aa, Homo sapiens
XTYLGGILNPIPDCQFELSGADGIVRSSQVEQEEKTKPGQAVDCIWTIKATPKAKIYLRFLDYQMEHSNECKRNFVAVYDGSSSIENLKAKFCSTVANDVMLKTGIGVIRMWADEGSRLSRFRMLFTSFVEQKKKAGVFEQITKTHGTIIGITSGIVLVLLIISILVQVKQPRKKVMACKTAFNKTGFQEVFDP

SEQ ID NO: 521 - protein, 228 aa, Homo sapiens
XSSSIENLKAKFCSTVANDVMLKTGIGVIRMWADEEKKKAGVFEQITKTHGTIIGITSGIVLVLLIISILVQVKQPRKKVMACKTAFNKTGFQEVFDPPHYELFSLRDKEISADLADLSEELDNYQKMRRSSTASRCIHDHHCGSQASSVKQSRTNLSSMELPFRNDFAQPQPMKTFNSTFKKSSYTFKQGHECPEQALEDRVMEEIPCEIYVRGREDSAQASISIDF

SEQ ID NO: 522 - protein, 347 aa, Homo sapiens
MSSWSRQRPKSPGGIQPHVSRTLFLLLLLAASAWGVTLSPKDCQVFRSDHGSSISCQPPAEIPGYLPADTVHLAVEFFNLTHLPANLLQGASKLQELHLSSNGLESLSPEFLRPVPQLRVLDLTRNALTGLPPGLFQASATLDTLVLKENQLEVLEVSWLHGLKALGHLDLSGNRLRKLPPGLLANFTLLRTLDLGENQLETLPPDLLRGPLQLERLHLEGNKLQVLGKDLLLPQPDLRYLFLNGNKLARVAAGAFQGLRQLDMLDLSNNSLASVPEGLWASLGQPNWDMRDGFDISGNPWICDQNLSDLYRWLQAQKDKMFSQNDTRCAGPEAVKGQTLLAVAKSQ

SEQ ID NO: 523 - protein, 330 aa, Homo sapiens
MRGPEPGPQPTMEGDVLDTLEALGYKGPLLEEQALTKAAEGGLSSPEFSELCIWLGSQIKSLCNLEESITSAGRDDLESFQLEISGFLKEMACPYSVLISGDIKDRLKKKEDCLKLLLFLSTELQASQILQNKKHKNSQLDKNSEVYQEVQAMFDTLGIPKSTTSDIPHMLNQVESKVKDILSKVQKNHVGKPLLKMDLNSEQAEQLERINDALSCEYECRRRMLMKRLDVTVQSFGWSDRAKVKTDDIARIYQPKRYALSPKTTITMAHLLAAREDLSKIIRTSSGTSREKTACAINKVGVSFSTVENELMISYLMFLQILVYFSFMSW

SEQ ID NO: 524 - protein, 101 aa, Homo sapiens
ISGDIKDRLKKKEDCLKLLLFLSTELQASQILQNKKHKNSQLDKNSEVYQEVQAMFDTLGIPKSTTSDIPHMLNQVESKVKDILSKIHMEEVVVVVVVVVE

SEQ ID NO: 525 - protein, 352 aa, Homo sapiens
MKPALLEVMRMNRICRMVLATCLGSFILVIFYFQSMLHPVMRRNPFGVDICCRKGSRSPLQELYNPIQLELSNTAVLHQMRRDQVTDTCRANSATSRKRRVLTPNDLKHLVVDEDHELIYCYVPKVACTNWKRLMMVLTGRGKYSDPMEIPANEAHVSANLKTLNQYSIPEINHRLKSYMKFLFVREPFERLVSAYRNKFTQKYNISFHKRYGTKIIKRQRKNATQEALRKGDDVKFEEFVAYLIDPHTQREEPFNEHWQTVYSLCHPCHIHYDLVGKYETLEEDSNYVLQLAGVGSYLKFPTYAKSTRTTDEMTTEFFQNISSEHQTQLYEVYKLDFLMFNYSVPSYLKLE

SEQ ID NO: 526 - protein, 72 aa, Homo sapiens
MKPALLEVMRMNRICRMVLATCLGSFILVIFYFQIMRRNPFGVDICCRKGSRSPLQELYNPIQVSFKHSVSM

SEQ ID NO: 527 - protein, 77 aa, Homo sapiens
MKPALLEVMRMNRICRMVLATCLGSFILVIFYFQSMLHPVMRRNPFGVDICCRKGSRSPLQELYNPIQVSFKHSVSM

SEQ ID NO: 528 - protein, 347 aa, Homo sapiens
MKPALLEVMRMNRICRMVLATCLGSFILVIFYFQIMRRNPFGVDICCRKGSRSPLQELYNPIQLELSNTAVLHQMRRDQVTDTCRANSATSRKRRVLTPNDLKHLVVDEDHELIYCYVPKVACTNWKRLMMVLTGRGKYSDPMEIPANEAHVSANLKTLNQYSIPEINHRLKSYMKFLFVREPFERLVSAYRNKFTQKYNISFHKRYGTKIIKRQRKNATQEALRKGDDVKFEEFVAYLIDPHTQREEPFNEHWQTVYSLCHPCHIHYDLVGKYETLEEDSNYVLQLAGVGSYLKFPTYAKSTRTTDEMTTEFFQNISSEHQTQLYEVYKLDFLMFNYSVPSYLKLE

SEQ ID NO: 529 - protein, 775 aa, Homo sapiens
MEPPYSLTAHYDEFQEVKYVSRCGAGGARGASLPPGFPLGAARSATGARSGLPRWNRREVCLLSGLVFAAGLCAILAAMLALKYLGPVAAGGGACPEGCPERKAFARAARFLAANLDASIDPCQDFYSFACGGWLRRHAIPDDKLTYGTIAAIGEQNEERLRRLLARPGGGPGGAAQRKVRAFFRSCLDMREIERLGPRPMLEVIEDCGGWDLGGAEERPGVAARWDLNRLLYKAQGVYSAAALFSLTVSLDDRNSSRYVIRIDQDGLTLPERTLYLAQDEDSEKILAAYRVFMERVLSLLGADAVEQKAQEILQVEQQLANITVSEHDDLRRDVSSMYNKVTLGQLQKITPHLRWKWLLDQIFQEDFSEEEEVVLLATDYMQQVSQLIRSTPHRVLHNYLVWRVVVVLSEHLSPPFREALHELAQEMEGSDKPQELARVCLGQANRHFGMALGALFVHEHFSAASKAKVQQLVEDIKYILGQRLEELDWMDAETRAAARAKLQYMMVMVGYPDFLLKPDAVDKEYEFEVHEKTYFKNILNSIRFSIQLSVKKIRQEVDKSTWLLPPQALNAYYLPNKNQMVFPAGILQPTLYDPDFPQSLNYGGIGTIIGHELTHGYDDWGGQYDRSGNLLHWWTEASYSRFLRKAECIVRLYDNFTVYNQRVNGKHTLGENIADMGGLKLAYHAYQKWVREHGPEHPLPRLKYTHDQLFFIAFAQNWCIKRRSQSIYLQVLTDKHAPEHYRVLGSVSQFEEFGRAFHCPKDSPMNPAHKCSVW

SEQ ID NO: 530 - protein, 773 aa, Homo sapiens
MEPPYSLTAHYDEFQEVKYVSRCGAGGARGASLPPGFPLGAARSATGARSGLPRWNRREVCLLSGLVFAAGLCAILAAMLALKYLGPVAAGGGACPEGCPERKAFARAARFLAANLDASIDPCQDFYSFACGGWLRRHAIPDDKLTYGTIAAIGEQNEERLRRLLARPGGGPGGAAQRKVRAFFRSCLDMREIERLGPRPMLEVIEDCGGWDLGGAEERPGVAARWDLNRLLYKAQGVYSAAALFSLTVSLDDRNSSRYVIRIDQDGLTLPERTLYLAQDEDSEKILAAYRVFMERVLSLLGADAVEQKAQEILQVEQQLANITVSEHDDLRRDVSSMYNKVTLGQLQKITPHLRWKWLLDQIFQEDFSEEEEVVLLATDYMQQVSQLIRSTPHRVLHNYLVWRVVVVLSEHLSPPFREALHELAQEMEGSDKPQELARVCLGQANRHFGMALGALFVHEHFSAASKAKVQQLVEDIKYILGQRLEELDWMDAETRAAARAKLQYMMVMVGYPDFLLKPDAVDKEYEFEVHEKTYFKNILNSIRFSIQLSVKKIRQEVDKWLLPPQALNAYYLPNKNQMVFPAGILQPTLYDPDFPQSLNYGGIGTIIGHELTHGYDDWGGQYDRSGNLLHWWTEASYSRFLRKAECIVRLYDNFTVYNQRVNGKHTLGENIADMGGLKLAYHAYQKWVREHGPEHPLPRLKYTHDQLFFIAFAQNWCIKRRSQSIYLQVLTDKHAPEHYRVLGSVSQFEEFGRAFHCPKDSPMNPAHKCSVW

SEQ ID NO: 531 - protein, 233 aa, Homo sapiens
MSQSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNNAAAESRKGQERFNRWFLTGMTVAGVVLLGSLFSRK

SEQ ID NO: 532 - protein, 170 aa, Homo sapiens
MSQSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQDTFVELYGNNAAAESRKGQERFNRWFLTGMTVAGVVLLGSLFSRK

SEQ ID NO: 533 - protein, 233 aa, Homo sapiens; identical to SEQ ID NO: 531

SEQ ID NO: 534 - protein, 813 aa, Homo sapiens
MSLLGDPLQALPPSAAPTGPLLAPPAGATLNRLREPLLRRLSELLDQAPEGRGWRRLAELAGSRGRLRLSCLDLEQCSLKVLEPEGSPSLCLLKLMGEKGCTVTELSDFLQAMEHTEVLQLLSPPGIKITVNPESKAVLAGQFVKLCCRATGHPFVQYQWFKMNKEIPNGNTSELIFNAVHVKDAGFYVCRVNNNFTFEFSQWSQLDVCDIPESFQRSVDGVSESKLQICVEPTSQKLMPGSTLVLQCVAVGSPIPHYQWFKNELPLTHETKKLYMVPYVDLEHQGTYWCHVYNDRDSQDSKKVEIIIDELNNLGHPDNKEQTTDQPLAKDKVALLIGNMNYREHPKLKAPLVDVYELTNLLRQLDFKVVSLLDLTEYEMRNAVDEFLLLLDKGVYGLLYYAGHGYENFGNSFMVPVDAPNPYRSENCLCVQNILKLMQEKETGLNVFLLDMCRKRNDYDDTIPILDALKVTANIVFGYATCQGAEAFEIQHSGLANGIFMKFLKDRLLEDKKITVLLDEVAEDMGKCHLTKGKQALEIRSSLSEKRALTDPIQGTEYSAESLVRNLQWAKAHELPESMCLKFDCGVQIQLGFAAEFSNVMIIYTSIVYKPPEIIMCDAYVTDFPLDLDIDPKDANKGTPEETGSYLVSKDLPKHCLYTRLSSLQKLKEHLVFTVCLSYQYSGLEDTVEDKQEVNVGKPLIAKLDMHRGLGRKTCFQTCLMSNGPYQSSAATSGGAGHYHSLQDPFHGVYHSHPGNPSNVTPADSCHCSRTPDAFISSFAHHASCHFSRSNVPVETTDEIPFSFSDRLRISEK

SEQ ID NO: 535 - protein, 824 aa, Homo sapiens
MSLLGDPLQALPPSAAPTGPLLAPPAGATLNRLREPLLRRLSELLDQAPEGRGWRRLAELAGSRGRLRLSCLDLEQCSLKVLEPEGSPSLCLLKLMGEKGCTVTELSDFLQAMEHTEVLQLLSPPGIKITVNPESKAVLAGQFVKLCCRATGHPFVQYQWFKMNKEIPNGNTSELIFNAVHVKDAGFYVCRVNNNFTFEFSQWSQLDVCDIPESFQRSVDGVSESKLQICVEPTSQKLMPGSTLVLQCVAVGSPIPHYQWFKNELPLTHETKKLYMVPYVDLEHQGTYWCHVYNDRDSQDSKKVEIIIGRTDEAVECTEDELNNLGHPDNKEQTTDQPLAKDKVALLIGNMNYREHPKLKAPLVDVYELTNLLRQLDFKVVSLLDLTEYEMRNAVDEFLLLLDKGVYGLLYYAGHGYENFGNSFMVPVDAPNPYRSENCLCVQNILKLMQEKETGLNVFLLDMCRKRNDYDDTIPILDALKVTANIVFGYATCQGAEAFEIQHSGLANGIFMKFLKDRLLEDKKITVLLDEVAEDMGKCHLTKGKQALEIRSSLSEKRALTDPIQGTEYSAESLVRNLQWAKAHELPESMCLKFDCGVQIQLGFAAEFSNVMIIYTSIVYKPPEIIMCDAYVTDFPLDLDIDPKDANKGTPEETGSYLVSKDLPKHCLYTRLSSLQKLKEHLVFTVCLSYQYSGLEDTVEDKQEVNVGKPLIAKLDMHRGLGRKTCFQTCLMSNGPYQSSAATSGGAGHYHSLQDPFHGVYHSHPGNPSNVTPADSCHCSRTPDAFISSFAHHASCHFSRSNVPVETTDEIPFSFSDRLRISEK

SEQ ID NO: 536 - protein, 119 aa, Homo sapiens
MGEKGCTVTELSDFLQAMEHTEVLQLLSPPGIKITVNPESKAVLAGQFVKLCCRATGHPFVQYQWFKMNKEIPNGNTSELIFNAVHVKDAGFYVCRVNNNFTFEFSQWSQLDVCDIPES

SEQ ID NO: 537 - protein, 506 aa, Homo sapiens
MAGEVSAATGRFSLERLGLPGLALAAALLLLALCLLVRRTRRPGEPPLIKGWLPYLGVVLNLRKDPLRFMKTLQKQHGDTFTVLLGGKYITFILDPFQYQLVIKNHKQLSFRVFSNKLLEKAFSISQLQKNHDMNDELHLCYQFLQGKSLDILLESMMQNLKQVFEPQLLKTTSWDTAELYPFCSSIIFEITFTTIYGKVIVCDNNKFISELRDDFLKFDDKFAYLVSNIPIELLGNVKSIREKIIKCFSSEKLAKMQGWSEVFQSRQDVLEKYYVHEDLEIGAHHLGFLWASVANTIPTMFWAMYYLLRHPEAMAAVRDEIDRLLQSTGQKKGSGFPIHLTREQLDSLICLESSIFEALRLSSYSTTIRFVEEDLTLSSETGDYCVRKGDLVAIFPPVLHGDPEIFEAPEEFRYDRFIEDGKKKTTFFKRGKKLKCYLMPFGTGTSKCPGRFFALMEIKQLLVILLTYFDLEIIDDKPIGLNYSRLLFGIQYPDSDVLFRYKVKS

SEQ ID NO: 538 - protein, 543 aa, Homo sapiens
MLLRSKPALPPPLMLLLLGPLGPLSPGALPRPAQAQDVVDLDFFTQEPLHLVSPSFLSVTIDANLATDPRFLILLGSPKLRTLARGLSPAYLRFGGTKTDFLIFDPKKESTFEERSYWQSQVNQDICKYGSIPPDVEEKLRLEWPYQEQLLLREHYQKKFKNSTYSRSSVDVLYTFANCSGLDLIFGLNALLRTADLQWNSSNAQLLLDYCSSKGYNISWELGNEPNSFLKKADIFINGSQLGEDFIQLHKLLRKSTFKNAKLYGPDVGQPRRKTAKMLKSFLKAGGEVIDSVTWHHYYLNGRTATKEDFLNPDVLDIFISSVQKVFQVVESTRPGKKVWLGETSSAYGGGAPLLSDTFAAGFMWLDKLGLSARMGIEVVMRQVFFGAGNYHLVDENFDPLPDYWLSLLFKKLVGTKVLMASVQGSKRRKLRVYLHCTNTDNPRYKEGDLTLYAINLHNVTKYLRLPYPFSNKQVDKYLLRPLGPHGLLSKSVQLNGLTLKMVDDQTLPPLMEKPLRPGSSLGLPAFSYSFFVIRNAKVAACI

SEQ ID NO: 539 - protein, 543 aa, Homo sapiens; identical to SEQ ID NO: 538

SEQ ID NO: 540 - protein, 226 aa, Homo sapiens
MLLRSKPALPPPLMLLLLGPLGPLSPGALPRPAQAQDVVDLDFFTQEPLHLVSPSFLSVTIDANLATDPRFLILLGSPKLRTLARGLSPAYLRFGGTKTDFLIFDPKKESTFEERSYWQSQVNQDICKYGSIPPDVEEKLRLEWPYQEQLLLREHYQKKFKNSTYSRSSVDVLYTFANCSGLDLIFGLNALLRTADLQWNSSNAQLLLDYCSSKGYNISWELGNAS

SEQ ID NO: 541 - protein, 168 aa, Homo sapiens
MLLRSKPALPPPLMLLLLGPLGPLSPGALPRPAQAQDVVDLDFFTQEPLHLVSPSFLSVTIDANLATDPRFLILLGSPKLRTLARGLSPAYLRFGGTKTDFLIFDPKKESTFEERSYWQSQVNQDICKYGSIPPDVEEKLRLEWPYQEQLLLREHYQKKFKNSTYSTS

SEQ ID NO: 542 - protein, 380 aa, Homo sapiens
MLLRSKPALPPPLMLLLLGPLGPLSPGALPRPAQAQDVVDLDFFTQEPLHLVSPSFLSVTIDANLATDPRFLILLGSPKLRTLARGLSPAYLRFGGTKTDFLIFDPKKESTFEERSYWQSQVNQDICKYGSIPPDVEEKLRLEWPYQEQLLLREHYQKKFKNSTYSRSSVDVLYTFANCSGLDLIFGLNALLRTADLQWNSSNAQLLLDYCSSKGYNISWELGNEPNSFLKKADIFINGSQLGEDFIQLHKLLRKSTFKNAKLYGPDVGQPRRKTAKMLKSFLKAGGEVIDSVTWHHYYLNGRTATKEDFLNPDVLDIFISSVQKVFQVVESTRPGKKVWLGETSSAYGGGAPLLSDTFAAGFMIIGYLFCSRNWWAPRC

SEQ ID NO: 543 - protein, 469 aa, Homo sapiens
MLLRSKPALPPPLMLLLLGPLGPLSPGALPRPAQAQDVVDLDFFTQEPLHLVSPSFLSVTIDANLATDPRFLILLGSPKLRTLARGLSPAYLRFGGTKTDFLIFDPKKESTFEERSYWQSQVNQDICKYGSIPPDVEEKLRLEWPYQEQLLLREHYQKKFKNSTYSRSSVDVLYTFANCSGLDLIFGLNALLRTADLQWNSSNAQLLLDYCSSKGYNISWELGNEPNSFLKKADIFINGSQLGEDFIQLHKLLRKSTFKNAKLYGPDVGQPRRKTAKMLKSFLKAGGEVIDSVTWHHYYLNGRTATKEDFLNPDVLDIFISSVQKVFQDYWLSLLFKKLVGTKVLMASVQGSKRRKLRVYLHCTNTDNPRYKEGDLTLYAINLHNVTKYLRLPYPFSNKQVDKYLLRPLGPHGLLSKSVQLNGLTLKMVDDQTLPPLMEKPLRPGSSLGLPAFSYSFFVIRNAKVAACI

SEQ ID NO: 544 - protein, 485 aa, Homo sapiens
MLLRSKPALPPPLMLLLLGPLGPLSPGALPRPAQAQDVVDLDFFTQEPLHLVSPSFLSVTIDANLATDPRFLILLGSPKLRTLARGLSPAYLRFGGTKTDFLIFDPKKESTFEERSYWQSQVNQDICKYGSIPPDVEEKLRLEWPYQEQLLLREHYQKKFKNSTYSKPNSFLKKADIFINGSQLGEDFIQLHKLLRKSTFKNAKLYGPDVGQPRRKTAKMLKSFLKAGGEVIDSVTWHHYYLNGRTATKEDFLNPDVLDIFISSVQKVFQVVESTRPGKKVWLGETSSAYGGGAPLLSDTFAAGFMWLDKLGLSARMGIEVVMRQVFFGAGNYHLVDENFDPLPDYWLSLLFKKLVGTKVLMASVQGSKRRKLRVYLHCTNTDNPRYKEGDLTLYAINLHNVTKYLRLPYPFSNKQVDKYLLRPLGPHGLLSKSVQLNGLTLKMVDDQTLPPLMEKPLRPGSSLGLPAFSYSFFVIRNAKVAACI

SEQ ID NO: 545 - protein, 524 aa, Homo sapiens
MDTESTYSGYSYYSSHSKKSHRQGERTRERHKSPRNKDGRGSEKSVTIQPPTGEPLLGNDSTRTEEVQDDNWGETTTAITGTSEHSISQEDIARISKDMEDSVGLDCKRYLGLTVASFLGLLVFLTPIAFILLPPILWRDELEPCGTICEGLFISMAFKLLILLIGTWALFFRKRRADMPRVFVFRALLLVLIFLFVVSYWLFYGVRILDSRDRNYQGIVQYAVSLVDALLFIHYLAIVLLELRQLQPMFTLQVVRSTDGESRFYSLGHLSIQRAALVVLENYYKDFTIYNPNLLTASKFRAAKHMAGLKVYNVDGPSNNATGQSRAMIAAAARRRDSSHNELYYEEAEHERRVKKRKARLVVAVEEAFIHIQRLQAEEQQKAPGEVMDPREAAQAIFPSMARALQKYLRITRQQNYHSMESILQHLAFCITNGMTPKAFLERYLSAGPTLQYDKDRWLSTQWRLVSDEAVTNGLRDGIVFVLKCLDFSLVVNVKKIPFIILSEEFIDPKSHKFVLRLQSETSV

SEQ ID NO: 546 - protein, 524 aa, Homo sapiens; identical to SEQ ID NO: 545

SEQ ID NO: 547 - protein, 524 aa, Homo sapiens; identical to SEQ ID NO: 545

SEQ ID NO: 548 - protein, 522 aa, Homo sapiens
MDTESTYSGYSYYSSHSKKSHRQGERTRERHKSPRNKDGRGSEKSVTIQPPTGEPLLGNDSTRTEEDDNWGETTTAITGTSEHSISQEDIARISKDMEDSVGLDCKRYLGLTVASFLGLLVFLTPIAFILLPPILWRDELEPCGTICEGLFISMAFKLLILLIGTWALFFRKRRADMPRVFVFRALLLVLIFLFVVSYWLFYGVRILDSRDRNYQGIVQYAVSLVDALLFIHYLAIVLLELRQLQPMFTLQVVRSTDGESRFYSLGHLSIQRAALVVLENYYKDFTIYNPNLLTASKFRAAKHMAGLKVYNVDGPSNNATGQSRAMIAAAARRRDSSHNELYYEEAEHERRVKKRKARLVVAVEEAFIHIQRLQAEEQQKAPGEVMDPREAAQAIFPSMARALQKYLRITRQQNYHSMESILQHLAFCITNGMTPKAFLERYLSAGPTLQYDKDRWLSTQWRLVSDEAVTNGLRDGIVFVLKCLDFSLVVNVKKIPFIILSEEFIDPKSHKFVLRLQSETSV

SEQ ID NO: 549 - protein, 240 aa, Homo sapiens
MAGPPRLLLLPLLLALARGLPGALAAQEVQQSPHCTTVPVGASVNITCSTSGGLRGIYLRQLGPQPQDIIYYEDGVVPTTDRRFRGRIDFSGSQDNLTITMHRLQLSDTGTYTCQAITEVNVYGSGTLVLVTEEQSQGWHRCSDAPPRASALPAPPTGSALPDPQTASALPDPPAASALPAALAVISFLLGLGLGVACVLARTQIKKLCSWRDKNSAACVVYEDMSHSRCNTLSSPNQYQ

SEQ ID NO: 550 - protein, 121 aa, Homo sapiens
MHRLQLSDTGTYTCQAITEVNVYGSGTLVLVTEEQSQGWHRCSDAPPRASALPAPPTGSALPDPQTASALPDPPAASALPAALAVISFLLGLGLGVACVLARTQVSVSPSCHLHPKDCSLS

SEQ ID NO: 551 - protein, 35 aa, Homo sapiens
MAGPPRLLLLPLLLALARGLPGALAAQAGACVGST

SEQ ID NO: 552 - protein, 160 aa, Homo sapiens
MAGPPRLLLLPLLLALARGLPGALAAQGRTFSVLLARLMVTAQVLPRGAAVSPLHDCPRGSLRQHHLLHQRGPAWDLPEAARATAPRHHLLRGRGGAHYGQTVPGPHRLLRVPGQPDYHHAPPAAVGHWHLHLPGHHGGQCLRLRHPGPGDRGTVPRMAQ

SEQ ID NO: 553 - protein, 140 aa, Homo sapiens
MHRLQLSDTGTYTCQAITEVNVYGSGTLVLVTEEQSQGWHRCSDAPPRASALPAPPTGSALPDPQTASALPDPPAASALPAALAVISFLLGLGLGVACVLARTQIKKLCSWRDKNSAACVVYEDMSHSRCNTLSSPNQYQ

SEQ ID NO: 554 - protein, 221 aa, Homo sapiens
MAGPPRLLLLPLLLALARGLPGALAAQEVQQSPHCTTVPVGASVNITCSTSGGLRGIYLRQLGPQPQDIIYYEDGVVPTTDRRFRGRIDFSGSQDNLTITMHRLQLSDTGTYTCQAITEVNVYGSGTLVLVTEEQSQGWHRCSDAPPRASALPAPPTGSALPDPQTASALPDPPAASALPAALAVISFLLGLGLGVACVLARTQVSVSPSCHLHPKDCSLS

SEQ ID NO: 555 - protein, 40 aa, Homo sapiens
XNYEMPRGDTSSLRAPAEDLRWVEKTLSLLYSPQVAGSSR

SEQ ID NO: 556 - protein, 107 aa, Homo sapiens
MQCQGYVALLGSDDQSWGWNLVDNNLLHNGEVNGSFPQCNNAPKYQIGERIRVILDMEDKTLAFERGYEFLGVAFRGLPKVCLYPAVSAVYGNTEVTLVYLGKPLDG

SEQ ID NO: 557 - protein, 530 aa, Homo sapiens
MSRSPQRALPPGALPRLLQAAPAAAPRALLPQWPRRPGRRWPASPLGMKVFRRKALVLCAGYALLLVLTMLNLLDYKWHKEPLQQCNPDGPLGAAAGAAGGSWGRPGPPPAGPPRAHARLDLRTPYRPPAAAVGAAPAAAAGMAGVAAPPGNGTRGTGGVGDKRQLVYVFTTWRSGSSFFGELFNQNPEVFFLYEPVWHVWQKLYPGDAVSLQGAARDMLSALYRCDLSVFQLYSPAGSGGRNLTTLGIFGAATNKVVCSSPLCPAYRKEVVGLVDDRVCKKCPPQRLARFEEECRKYRTLVIKGVRVFDVAVLAPLLRDPALDLKVIHLVRDPRAVASSRIRSRHGLIRESLQVVRSRDPRAHRMPFLEAAGHKLGAKKEGVGGPADYHALGAMEVICNSMAKTLQTALQPPDWLQGHYLVVRYEDLVGDPVKTLRRVYDFVGLLVSPEMEQFALNMTSGSGSSSKPFVVSARNATQAANAWRTALTFQQIKQVEEFCYQPMAVLGYERVNSPEEVKDLSKTLLRKPRL

SEQ ID NO: 558 - protein, 84 aa, Homo sapiens
MKQTQVGSLFSLGIRNPEPGPVSGTAVPRQLAWKSGKYVMVMGVVQACSPEPCLQAVKMTDLSDNPIHESMWELEVEDLHRNIP

SEQ ID NO: 559 - protein, 313 aa, Homo sapiens
MALLVDRVRGHWRIAAGLLFNLLVSICIVFLNKWIYVYHGFPNMSLTLVHFVVTWLGLYICQKLDIFAPKSLPPSRLLLLALSFCGFVVFTNLSLQNNTIGTYQLAKAMTTPVIIAIQTFCYQKTFSTRIQLTLIPITLGVILNSYYDVKFNFLGMVFAALGVLVTSLYQVWVGAKQHELQVNSMQLLYYQAPMSSAMLLVAVPFFEPVFGEGGIFGPWSVSALLMVLLSGVIAFMVNLSIYWIIGNTSPVTYNMFGHFKFCITLFGGYVLFKDPLSINQALGILCTLFGILAYTHFKLSEQEGSRSKLAQRP

SEQ ID NO: 560 - protein, 125 aa, Homo sapiens
QAPMSSAMLLVAVPFFEPVFGEGGIFGPWSVSALSTRLECSGTIMAHCSLSLLGSSNPPASTSPVAGTTGAHQHTWLIFIFFVETEFCHVAQADLKLLSSSHLSASASRSAGITGLSHHTWPVIS

SEQ ID NO: 561 - protein, 34 aa, Homo sapiens
MTVMSLSRDLKDDFHSDTVLSILNEQRIRGILCD

SEQ ID NO: 562 - protein, 193 aa, Homo sapiens
MAEAEESPGDPGTASPRPLMRDLKAVGKKFMHVLYPRKSNTLLRDWDLWGPLILCVTLALMLQRDSADSEKDGGPQFAEVFVIVWFGAVTITLNSKLLGGNISFFQSLCVLGYCILPLTVAMLICRLVLLADPGPVNFMVRLFVVIVMFAWSIVASTAFLADSQPPNRRALAVYPVFLFYFVISWMILTFTPQ

SEQ ID NO: 563 - protein, 188 aa, Homo sapiens
MAEAEESPGDPGTASPRPLMRDLKAVGKKFMHVLYPRKSNTLLRDWDLWGPLILCVTLALMLQRDSADSEKDGGPQFAEVFVIVWFGAVTITLNSKLLGGNISFFQSLCVLGYCILPLTVAMLICRLVLLADPGPVNFMVRLFVVIVMFAWSIVASTAFLADSQPPNRRALAVYPVFLFYFVISWMIL

SEQ ID NO: 564 - protein, 236 aa, Homo sapiens
MAEAEESPGDPGTASPRPLFAGLSDISISQDIPVEGEITIPMRSRIREFDSSTLNESVRNTIMRDLKAVGKKFMHVLYPRKSNTLLRDWDLWGPLILCVTLALMLQRDSADSEKDGGPQFAEVFVIVWFGAVTITLNSKLLGGNISFFQSLCVLGYCILPLTVAMLICRLVLLADPGPVNFMVRLFVVIVMFAWSIVASTAFLADSQPPNRRALAVYPVFLFYFVISWMILTFTPQ

SEQ ID NO: 565 - protein, 520 aa, Homo sapiens
MEVLESGEQGVLQWDRKLSELSEPGDGEALMYHTHFSELLDEFSQNVLGQLLNDPFLSEKSVSMEVEPSPTSPAPLIQAEHSYSLCEEPRAQSPFTHITTSDSFNDDEVESEKWYLSTDFPSTSIKTEPVTDEPPPGLVPSVTLTITAISTPLEKEEPPLEMNTGVDSSCQTIIPKIKLEPHEVDQFLNFSPKEAPVDHLHLPPTPPSSHGSDSEGSLSPNPRLHPFSLPQTHSPSRAAPRAPSALSSSPLLTAPHKLQGSGPLVLTEEEKRTLIAEGYPIPTKLPLSKSEEKALKKIRRKIKNKISAQESRRKKKEYMDSLEKKVESCSTENLELRKKVEVLENTNRTLLQQLQKLQTLVMGKVSRTCKLAGTQTGTCLMVVVLCFAVAFGSFFQGYGPYPSATKMALPSQHSLQEPYTASVVRSRNLLIYEEHSPPEESSSPGSAGELGGWDRGSSLLRVSGLESRPDVDLPHFIISNETSLEKSVLLELQQHLVSAKLEGNETLKVVELDRRVNTTF

SEQ ID NO: 566 - protein, 240 aa, Homo sapiens [sequence omitted]

SEQ ID NO: 567 - protein, 460 aa, Homo sapiens
MEVLESGEQGVLQWDRKLSELSEPGDGEALMYHTHFSELLDEFSQNVLGQLLNDPFLSEKSVSMEVEPSPTSPAPLIQAEHSYSLCEEPRAQSPFTHITTSDSFNDDEVESEKWYLSTDFPSTSIKTEPVTDEPPPGLVPSVTLTITAISTPLEKEEPPLEMNTGVDSSCQTIIPKIKLEPHEVDQFLNFSPKEAPVDHLHLPPTPPSSHGSDSEGSLSPNPRLHPFSLPQTHSPSRAAPRAPSALSSSPLLTAPHKLQGSGPLVLTEEEKRTLIAEGYPIPTKLPLSKSEEKALKKIRRKIKNKISAQESRRKKKEYMDSLEKKVESCSTENLELRKKVEVLENTNRTLLQQLQKLQTLVMGKVSRTCKLAGTQTGTCLMVVVLCFAVAFGSFFQGYGPYPSATKMALPSQHSLQEPYTASVGKTACGKLGRVLFYFPRAGFLSLPKGIFCESPMFKKW

SEQ ID NO: 568 - protein, 449 aa, Homo sapiens
MDRVYEIPEEPNVDPVSSLEEDVIRGANPRFTFPFSILFSTFLYCGEAASALYMVRIYRKNSETYWMTYTFSFFMFSSIMVQLTLIFVHRDLAKDKPLSLFMHLILLGPVIRCLEAMIKYLTLWKKEEQEEPYVSLTRKKMLIDGEEVLIEWEVGHSIRTLAMHRNAYKRMSQIQAFLGSVPQLTYQLYVSLISAEVPLGRVVLMVFSLVSVTYGATLCNMLAIQIKYDDYKIRLGPLEVLCITIWRTLEITSRLLILVLFSATLKLKAVPFLVLNFLIILFEPWIKFWRSGAQMPNNIEKNFSRVGTLVVLISVTILYAGINFSCWSALQLRLADRDLVDKGQNWGHMGLHYSVRLVENVIMVLVFKFFGVKVLLNYCHSLIALQLIIAYLISIGFMLLFFQYLHPLRSLFTHNVVDYLHCVCCHQHPRTRVENSEPPFETEARQSVV

SEQ ID NO: 569 - protein, 139 aa, Homo sapiens
MDRVYEIPEEPNVDPVSSLEEDVIRGANPRFTFPFSILFSTFLYCGEAASALYMVRIYRKNSETYWMTYTFSFFMFSSIMVQLTLIFVHRDLAKDKPLSLFMHLILLGPVISCANGIFPGICHLWGHPLQYVGYPDQVR

SEQ ID NO: 570 - protein, 471 aa, Homo sapiens
MASVVLPSGSQCAAAAAAAAPPGLRLRLLLLLFSAAALIPTGDGQNLFTKDVTVIEGEVATISCQVNKSDDSVIQLLNPNRQTIYFRDFRPLKDSRFQLLNFSSSELKVSLTNVSISDEGRYFCQLYTDPPQESYTTITVLVPPRNLMIDIQKDTAVEGEEIEVNCTAMASKPATTIRWFKGNTELKGKSEVEEWSDMYTVTSQLMLKVHKEDDGVPVICQVEHPAVTGNLQTQRYLEVQYKPQVHIQMTYPLQGLTREGDALELTCEAIGKPQPVMVTWVRVDDEMPQHAVLSGPNLFINNLNKTDNGTYRCEASNIVGKAHSDYMLYVYDPPTTIPPPTTTTTTTTTTTTTILTIITDTTATTEPAVHGLTQLPNSAEELDSEDLSDSRAGEEGSIRAVDHAVIGGVVAVVVFAMLCLLIILGRYFARHKGTYFTHEAKGADDAADADTAIINAEGGQNNSEEKKEYFI

SEQ ID NO: 571 - protein, 442 aa, Homo sapiens
MASVVLPSGSQCAAAAAAAAPPGLRLRLLLLLFSAAALIPTGDGQNLFTKDVTVIEGEVATISCQVNKSDDSVIQLLNPNRQTIYFRDFRPLKDSRFQLLNFSSSELKVSLTNVSISDEGRYFCQLYTDPPQESYTTITVLVPPRNLMIDIQKDTAVEGEEIEVNCTAMASKPATTIRWFKGNTELKGKSEVEEWSDMYTVTSQLMLKVHKEDDGVPVICQVEHPAVTGNLQTQRYLEVQYKPQVHIQMTYPLQGLTREGDALELTCEAIGKPQPVMVTWVRVDDEMPQHAVLSGPNLFINNLNKTDNGTYRCEASNIVGKAHSDYMLYVYDPPTTIPPPTTTTTTTTTTTTTILTIITDSRAGEEGSIRAVDHAVIGGVVAVVVFAMLCLLIILGRYFARHKGTYFTHEAKGADDAADADTAIINAEGGQNNSEEKKEYFI

SEQ ID NO: 572 - protein, 443 aa, Homo sapiens
MASVVLPSGSQCAAAAAAAAPPGLRLRLLLLLFSAAALIPTGDGQNLFTKDVTVIEGEVATISCQVNKSDDSVIQLLNPNRQTIYFRDFRPLKDSRFQLLNFSSSELKVSLTNVSISDEGRYFCQLYTDPPQESYTTITVLVPPRNLMIDIQKDTAVEGEEIEVNCTAMASKPATTIRWFKGNTELKGKSEVEEWSDMYTVTSQLMLKVHKEDDGVPVICQVEHPAVTGNLQTQRYLEVQYKPQVHIQMTYPLQGLTREGDALELTCEAIGKPQPVMVTWVRVDDEMPQHAVLSGPNLFINNLNKTDNGTYRCEASNIVGKAHSDYMLYVYDTTATTEPAVHGLTQLPNSAEELDSEDLSDSRAGEEGSIRAVDHAVIGGVVAVVVFAMLCLLIILGRYFARHKGTYFTHEAKGADDAADADTAIINAEGGQNNSEEKKEYFI

SEQ ID NO: 573 - protein, 453 aa, Homo sapiens
MASVVLPSGSQCAAAAAAAAPPGLRLRLLLLLFSAAALIPTGDGQNLFTKDVTVIEGEVATISCQVNKSDDSVIQLLNPNRQTIYFRDFRPLKDSRFQLLNFSSSELKVSLTNVSISDEGRYFCQLYTDPPQESYTTITVLVPPRNLMIDIQKDTAVEGEEIEVNCTAMASKPATTIRWFKGNTELKGKSEVEEWSDMYTVTSQLMLKVHKEDDGVPVICQVEHPAVTGNLQTQRYLEVQYKPQVHIQMTYPLQGLTREGDALELTCEAIGKPQPVMVTWVRVDDEMPQHAVLSGPNLFINNLNKTDNGTYRCEASNIVGKAHSDYMLYVYDPPTTIPPPTTTTTTTTTTTTTILTIITDTTATTEPAVHDSRAGEEGSIRAVDHAVIGGVVAVVVFAMLCLLIILGRYFARHKGTYFTHEAKGADDAADADTAIINAEGGQNNSEEKKEYFI

SEQ ID NO: 574 - protein, 67 aa, Homo sapiens
MASVVLPSGSQCAAAAAAAAPPGLRLRLLLLLFSAAALIPTGLTSSPRLECGGTISAHCSLGLPVLR

SEQ ID NO: 575 - protein, 414 aa, Homo sapiens
MASVVLPSGSQCAAAAAAAAPPGLRLRLLLLLFSAAALIPTGDGQNLFTKDVTVIEGEVATISCQVNKSDDSVIQLLNPNRQTIYFRDFRPLKDSRFQLLNFSSSELKVSLTNVSISDEGRYFCQLYTDPPQESYTTITVLVPPRNLMIDIQKDTAVEGEEIEVNCTAMASKPATTIRWFKGNTELKGKSEVEEWSDMYTVTSQLMLKVHKEDDGVPVICQVEHPAVTGNLQTQRYLEVQYKPQVHIQMTYPLQGLTREGDALELTCEAIGKPQPVMVTWVRVDDEMPQHAVLSGPNLFINNLNKTDNGTYRCEASNIVGKAHSDYMLYVYDSRAGEEGSIRAVDHAVIGGVVAVVVFAMLCLLIILGRYFARHKGTYFTHEAKGADDAADADTAIINAEGGQNNSEEKKEYFI

SEQ ID NO: 576 - protein, 111 aa, Homo sapiens
MIDIQKDTAVEGEEIEVNCTAMASKPATTIRWFKGNTELKGKSEVEEWSDMYTVTSQLMLKVHKEDDGVPVICQVEHPAVTGNLQTQRYLEVQYKPQVHIQMTYPLQGLTR

SEQ ID NO: 577 - protein, 79 aa, Homo sapiens
MIDIQKDTAVEGEEIEVNCTAMASKPATTIRWFKGNTELKGKSEVEEWSDMYTVTSQLMLKVHKEDDGVPVICQVEHPA

SEQ ID NO: 578 - protein, 413 aa, Homo sapiens
XSVVLPSGSQCAAAAAAAAPPGLRLRLLLLLFSAAALIPTGDGQNLFTKDVTVIEGEVATISCQVNKSDDSVIQLLNPNRQTIYFRDFRPLKDSRFQLLNFSSSELKVSLTNVSISDEGRYFCQLYTDPPQESYTTITVLVPPRNLMIDIQKDTAVEGEEIEVNCTAMASKPATTIRWFKGNTELKGKSEVEEWSDMYTVTSQLMLKVHKEDDGVPVICQVEHPAVTGNLQTQRYLEVQYKPQVHIQMTYPLQGLTREGDALELTCEAIGKPQPVMVTWVRVDDEMPQHAVLSGPNLFINNLNKTDNGTYRCEASNIVGKAHSDYMLYVYDSRAGEEGSIRAVDHAVIGGVVAVVVFAMLCLLIILGRYFARHKGTYFTHEAKGADDAADADTAIINAEGGQNNSEEKKEYFI

SEQ ID NO: 579 - protein, 346 aa, Homo sapiens
GDGQNLFTKDVTVIEGEVATISCQVNKSDDSVIQLLNPNRQTIYFRDFRPLKDSRFQLLNFSSSELKVSLTNVSISDEGRYFCQLYTDPPQESYTTITVLVPPRNLMIDIQKDTAVEGEEIEVNCTAMASKPATTIRWFKGNTELKGKSEVEEWSDMYTVTSQLMLKVHKEDDGVPVICQVEHPAVTGNLQTQRYLEVQYKPQVHIQMTYPLQGLTREGDALELTCEAIGKPQPVMVTWVRVDDEMPQHAVLSGPNLFINNLNKTDNGTYRCEASNIVGKAHSDYMLYVYDSRAGEEGSIRAVDHAVIGGVVAVVVFAMLCLLIILGRYFARHKGLFSLTSSPRIK

SEQ ID NO: 580 - protein, 373 aa, Homo sapiens
GDGQNLFTKDVTVIEGEVATISCQVNKSDDSVIQLLNPNRQTIYFRDFRPLKDSRFQLLNFSSSELKVSLTNVSISDEGRYFCQLYTDPPQESYTTITVLVPPRNLMIDIQKDTAVEGEEIEVNCTAMASKPATTIRWFKGNTELKGKSEVEEWSDMYTVTSQLMLKVHKEDDGVPVICQVEHPAVTGNLQTQRYLEVQYKPQVHIQMTYPLQGLTREGDALELTCEAIGKPQPVMVTWVRVDDEMPQHAVLSGPNLFINNLNKTDNGTYRCEASNIVGKAHSDYMLYVYDSRAGEEGSIRAVDHAVIGGVVAVVVFAMLCLLIILGRYFARHKGTYFTHEAKGADDAADADTAIINAEGGQNNSEEKKEYFI

SEQ ID NO: 581 - protein, 412 aa, Homo sapiens
GDGQNLFTKDVTVIEGEVATISCQVNKSDDSVIQLLNPNRQTIYFRDFRPLKDSRFQLLNFSSSELKVSLTNVSISDEGRYFCQLYTDPPQESYTTITVLVPPRNLMIDIQKDTAVEGEEIEVNCTAMASKPATTIRWFKGNTELKGKSEVEEWSDMYTVTSQLMLKVHKEDDGVPVICQVEHPAVTGNLQTQRYLEVQYKPQVHIQMTYPLQGLTREGDALELTCEAIGKPQPVMVTWVRVDDEMPQHAVLSGPNLFINNLNKTDNGTYRCEASNIVGKAHSDYMLYVYDPPTTIPPPTTTTTTTTTTTTTILTIITDTTATTEPAVHDSRAGEEGSIRAVDHAVIGGVVAVVVFAMLCLLIILGRYFARHKGTYFTHEAKGADDAADADTAIINAEGGQNNSEEKKEYFI

SEQ ID NO: 582 - protein, 430 aa, Homo sapiens
GDGQNLFTKDVTVIEGEVATISCQVNKSDDSVIQLLNPNRQTIYFRDFRPLKDSRFQLLNFSSSELKVSLTNVSISDEGRYFCQLYTDPPQESYTTITVLVPPRNLMIDIQKDTAVEGEEIEVNCTAMASKPATTIRWFKGNTELKGKSEVEEWSDMYTVTSQLMLKVHKEDDGVPVICQVEHPAVTGNLQTQRYLEVQYKPQVHIQMTYPLQGLTREGDALELTCEAIGKPQPVMVTWVRVDDEMPQHAVLSGPNLFINNLNKTDNGTYRCEASNIVGKAHSDYMLYVYDPPTTIPPPTTTTTTTTTTTTTILTIITDTTATTEPAVHGLTQLPNSAEELDSEDLSDSRAGEEGSIRAVDHAVIGGVVAVVVFAMLCLLIILGRYFARHKGTYFTHEAKGADDAADADTAIINAEGGQNNSEEKKEYFI

SEQ ID NO: 583 - protein, 401 aa, Homo sapiens
GDGQNLFTKDVTVIEGEVATISCQVNKSDDSVIQLLNPNRQTIYFRDFRPLKDSRFQLLNFSSSELKVSLTNVSISDEGRYFCQLYTDPPQESYTTITVLVPPRNLMIDIQKDTAVEGEEIEVNCTAMASKPATTIRWFKGNTELKGKSEVEEWSDMYTVTSQLMLKVHKEDDGVPVICQVEHPAVTGNLQTQRYLEVQYKPQVHIQMTYPLQGLTREGDALELTCEAIGKPQPVMVTWVRVDDEMPQHAVLSGPNLFINNLNKTDNGTYRCEASNIVGKAHSDYMLYVYDPPTTIPPPTTTTTTTTTTTTTILTIITDSRAGEEGSIRAVDHAVIGGVVAVVVFAMLCLLIILGRYFARHKGTYFTHEAKGADDAADADTAIINAEGGQNNSEEKKEYFI

SEQ ID NO: 584 - protein, 200 aa, Homo sapiens
MASSLTCTGVIWALLSFLCAATSCVGFFMPYWLWGSQLGKPVSFGTFRRCSYPVHDESRQMMVMVEECGRYASFQGIPSAEWRICTIVTGLGCGLLLLVALTALMGCCVSDLISRTVGRVAGGIQFLGGLLIGAGCALYPLGWDSEEVRQTCGYTSGQFDLGKCEIGWAYYCTGAGATAAMLLCTWLACFSGKKQKHYPY

SEQ ID NO: 585 - protein, 554 aa, Homo sapiens
MTAPGAAGRCPPTTWLGSLLLLVCLLASRSITEEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQDVVTKPDCNCLYPKAIPSSDPASVSPHQPLAPSMAPVAGLTWEDSEGTEGSSLLPGEQPLHTVDPGSAKQRPPRSTCQSFEPPETPVVKDSTIGGSPQPRPSVGAFNPGMEDILDSAMGTNWVPEEASGEASEIPVPQGTELSPSRPGGGSMQTEPARPSNFLSASSPLPASAKGQQPADVTGTALPRVGPVRPTGQDWNHTPQKTDHPSALLRDPPEPGSPRISSLRPQGLSNPSTLSAQPQLSRSHSSGSVLPLGELEGRRSTRDRRSPAEPEGGPASEGAARPLPRFNSVPLTDTGHERQSEGSFSPQLQESVFHLLVPSVILVLLAVGGLLFYRWRRRSHQEPQRADSPLEQPEGSPLTQDDRQVELPV

SEQ ID NO: 586 - protein, 119 aa, Homo sapiens
MTAPGAAGRCPPTTWLGSLLLLVCLLASRSITEEVSEYCSHMIGSGHLQSLQRLIDSQMETSCQITFEFVDQEQLKDPVCYLKKAFLLVQDIMEDTMRFRDNTPNAIAIVQLQELSLRL

SEQ ID NO: 587 - protein, 438 aa, Homo sapiens
MTAPGAAGRCPPTTWLGSLLLLVCLLASRSITEEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQDVVTKPDCNCLYPKAIPSSDPASVSPHQPLAPSMAPVAGLTWEDSEGTEGSSLLPGEQPLHTVDPGSAKQRPPRSTCQSFEPPETPVVKDSTIGGSPQPRPSVGAFNPGMEDILDSAMGTNWVPEEASGEASEIPVPQGTELSPSRPGGGSMQTEPARPSNFLSASSPLPASAKGQQPADVTGHERQSEGSFSPQLQESVFHLLVPSVILVLLAVGGLLFYRWRRRSHQEPQRADSPLEQPEGSPLTQDDRQVELPV

SEQ ID NO: 588 - protein, 554 aa, Homo sapiens; identical to SEQ ID NO: 585

SEQ ID NO: 589 - protein, 256 aa, Homo sapiens
MTAPGAAGRCPPTTWLGSLLLLVCLLASRSITEEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQGHERQSEGSFSPQLQESVFHLLVPSVILVLLAVGGLLFYRWRRRSHQEPQRADSPLEQPEGSPLTQDDRQVELPV

SEQ ID NO: 590 - protein, 285 aa, Homo sapiens
MTAPGAAGRCPPTIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQDVVTKPDCNCLYPKAIPSSDPASVSPHQPLAPSMAPVAGLTWEDSEGTEGSSLLPGEQPLHTVDPGSAKQRPPRSTCQSFEPPETPVVKDSTIGGSPQPRPSVGAFNPGMEDILDSAMGTNWVPEEASGEASEIPVPQGTELSPS

SEQ ID NO: 591 - protein, 113 aa, Homo sapiens
MIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFN

SEQ ID NO: 592 - protein, 150 aa, Homo sapiens
MGWGRLQRISLPSQCLSLLSTWLGSLLLLVCLLASRSITEEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPL

SEQ ID NO: 593 - protein, 173 aa, Homo sapiens
MARMNRPAPVEVSYKHMRFLITHNPTNATLSTFIEDLKKYGATTVVRVCEVTYDKTPLEKDGITVVDWPFDDGAPPPGKVVEDWLSLVKAKFCEAPGSCVAVHCVAGLGRAPVLVALALIESGMKYEDAIQFIRQKRRGAINSKQLTYLEKYRPKQRLRFKDPHTHKTRCCVM

SEQ ID NO: 594 - protein, 148 aa, Homo sapiens
MARMNRPAPVEVSYKHMRFLITHNPTNATLSTFIEDLKKYGATTVVRVCEVTYDKTPLEKDGITVVDWPFDDGAPPPGKVVEDWLSLVKAKFCEAPGSCVAVHCVAGLGRKRRGAINSKQLTYLEKYRPKQRLRFKDPHTHKTRCCVM

SEQ ID NO: 595 - protein, 148 aa, Homo sapiens; identical to SEQ ID NO: 594

SEQ ID NO: 596 - protein, 173 aa, Homo sapiens; identical to SEQ ID NO: 593

SEQ ID NO: 597 - protein, 70 aa, Homo sapiens
MARMNRPAPVEVSYKHMRFLITHNPTNATLSTFIEDLKKYGATTVVRVCEVTYDKTPLEKDGITVVDWPF

SEQ ID NO: 598 - protein, 87 aa, Homo sapiens
MARMNRPAPVEVSYKHMRFLITHNPTNATLSTFIESGMKYEDAIQFIRQKRRGAINSKQLTYLEKYRPKQRLRFKDPHTHKTRCCVM

SEQ ID NO: 599 - protein, 25 aa, Homo sapiens
MINACRDFLELAEIHSRKWQRALQY

SEQ ID NO: 600 - protein, 244 aa, Homo sapiens
MELTIFILRLAIYILTFPLYLLNFLGLWSWICKKWFPYFLVRFTVIYNEQMASKKRELFSNLQEFAGPSGKLSLLEVGCGTGANFKFYPPGCRVTCIDPNPNFEKFLIKSIAENRHLQFERFVVAAGENMHQVADGSVDVVVCTLVLCSVKNQERILREVCRVLRPGGAFYFMEHVAAECSTWNYFWQQVLDPAWHLLFDGCNLTRESWKALERASFSKLKLQHIQAPLSWELVRPHIYGYAVK

SEQ ID NO: 601 - protein, 244 aa, Homo sapiens; identical to SEQ ID NO: 600

SEQ ID NO: 602 - protein, 244 aa, Homo sapiens; identical to SEQ ID NO: 600

SEQ ID NO: 603 - protein, 244 aa, Homo sapiens
XELTIFILRLAIYILTFPLYLLNFLGLWSWICKKWFPYFLVRFTVIYNEQMASKKRELFSNLQEFAGPSGKLSLLEVGCGTGANFKFYPPGCRVTCIDPNPNFEKFLIKSIAENRHLQFERFVVAAGENMHQVADGSVDVVVCTLVLCSVKNQERILREVCRVLRPGGAFYFMEHVAAECSTWNYFWQQVLDPAWHLLFDGCNLTRESWKALERASFSKLKLQHIQAPLSWELVRPHIYGYAVK

SEQ ID NO: 604 - protein, 180 aa, Homo sapiens
MELTIFILRLAIYILTFPLYLLNFLGLWSWICKKWFPYFLVRFTVIYNEQMASKKRELFSNLQEFAGPSGKLSLLEVGCGTGANFKFYPPGCRVTCIDPNPNFEKFLIKSIAENRHLQFERFVVAAGENMHQVADGSVDVVVCTLVLCSVKNQERILREVCRVLRPGGAFYFMEHVAAEC

SEQ ID NO: 605 - protein, 811 aa, Homo sapiens
MLLLFHSKRMPVAEAPQVAGGQGDGGDGEEAEPEGMFKACEDSKRKARGYLRLVPLFVLLALLVLASAGVLLWYFLGYKAEVMVSQVYSGSLRVLNRHFSQDLTRRESSAFRSETAKAQKMLKELITSTRLGTYYNSSSVYSFGEGPLTCFFWFILQIPEHRRLMLSPEVVQALLVEELLSTVNSSAAVPYRAEYEVDPEGLVILEASVKDIAALNSTLGCYRYSYVGQGQVLRLKGPDHLASSCLWHLQGPKDLMLKLRLEWTLAECRDRLAMYDVAGPLEKRLITSVYGCSRQEPVVEVLASGAIMAVVWKKGLHSYYDPFVLSVQPVVFQACEVNLTLDNRLDSQGVLSTPYFPSYYSPQTHCSWHLTVPSLDYGLALWFDAYALRRQKYDLPCTQGQWTIQNRRLCGLRILQPYAERIPVVATAGITINFTSQISLTGPGVRVHYGLYNQSDPCPGEFLCSVNGLCVPACDGVKDCPNGLDERNCVCRATFQCKEDSTCISLPKVCDGQPDCLNGSDEEQCQEGVPCGTFTFQCEDRSCVKKPNPQCDGRPDCRDGSDEEHCDCGLQGPSSRIVGGAVSSEGEWPWQASLQVRGRHICGGALIADRWVITAAHCFQEDSMASTVLWTVFLGKVWQNSRWPGEVSFKVSRLLLHPYHEEDSHDYDVALLQLDHPVVRSAAVRPVCLPARSHFFEPGLHCWITGWGALREGGPISNALQKVDVQLIPQDLCSEVYRYQVTPRMLCAGYRKGKKDACQGDSGGPLVCKALSGRWFLAGLVSWGLGCGRPNYFGVYTRITGVISWIQQVVT

SEQ ID NO: 606 - protein, 824 aa, Homo sapiens
MPVAEAPQVAGGQGDGGDGEEAEPEGMFKACEDSKRKARGYLRLVPLFVLLALLVLASAGVLLWYFLGYKAEVMVSQVYSGSLRVLNRHFSQDLTRRESSAFRSETAKAQKMLKELITSTRLGTYYNSSSVYSFGEGPLTCFFWFILQIPEHRRLMLSPEVVQALLVEELLSTVNSSAAVPYRAEYEVDPEGLVILEASVKDIAALNSTLGCYRYSYVGQGQVLRLKGPDHLASSCLWHLQGPKDLMLKLRLEWTLAECRDRLAMYDVAGPLEKRLITSVYGCSRQEPVVEVLASGAIMAVVWKKGLHSYYDPFVLSVQPVVFQACEVNLTLDNRLDSQGVLSTPYFPSYYSPQTHCSWHLTVPSLDYGLALWFDAYALRRQKYDLPCTQGQWTIQNRRLCGLRILQPYAERIPVVATAGITINFTSQISLTGPGVRVHYGLYNQSDPCPGEFLCSVNGLCVPACDGVKDCPNGLDERNCVCRATFQCKEDSTCISLPKVCDGQPDCLNGSDEEQCQEGVPCGTFTFQCEDRSCVKKPNPQCDGRPDCRDGSDEEHCDCGLQGPSSRIVGGAVSSEGEWPWQASLQVRGRHICGGALIADRWVITAAHCFQEDSMASTVLWTVFLGKVWQNSRWPGEVSFKVSRLLLHPYHEEDSHDYDVALLQLDHPVVRSAAVRPVCLPARSHFFEPGLHCWITGWGALREGALRADAVALFYGWRNQGSETCCCPISNALQKVDVQLIPQDLCSEVYRYQVTPRMLCAGYRKGKKDACQGDSGGPLVCKALSGRWFLAGLVSWGLGCGRPNYFGVYTRITGVISWIQQVVT

SEQ ID NO: 607 - protein, 802 aa, Homo sapiens
MPVAEAPQVAGGQGDGGDGEEAEPEGMFKACEDSKRKARGYLRLVPLFVLLALLVLASAGVLLWYFLGYKAEVMVSQVYSGSLRVLNRHFSQDLTRRESSAFRSETAKAQKMLKELITSTRLGTYYNSSSVYSFGEGPLTCFFWFILQIPEHRRLMLSPEVVQALLVEELLSTVNSSAAVPYRAEYEVDPEGLVILEASVKDIAALNSTLGCYRYSYVGQGQVLRLKGPDHLASSCLWHLQGPKDLMLKLRLEWTLAECRDRLAMYDVAGPLEKRLITSVYGCSRQEPVVEVLASGAIMAVVWKKGLHSYYDPFVLSVQPVVFQACEVNLTLDNRLDSQGVLSTPYFPSYYSPQTHCSWHLTVPSLDYGLALWFDAYALRRQKYDLPCTQGQWTIQNRRLCGLRILQPYAERIPVVATAGITINFTSQISLTGPGVRVHYGLYNQSDPCPGEFLCSVNGLCVPACDGVKDCPNGLDERNCVCRATFQCKEDSTCISLPKVCDGQPDCLNGSDEEQCQEGVPCGTFTFQCEDRSCVKKPNPQCDGRPDCRDGSDEEHCDCGLQGPSSRIVGGAVSSEGEWPWQASLQVRGRHICGGALIADRWVITAAHCFQEDSMASTVLWTVFLGKVWQNSRWPGEVSFKVSRLLLHPYHEEDSHDYDVALLQLDHPVVRSAAVRPVCLPARSHFFEPGLHCWITGWGALREGGPISNALQKVDVQLIPQDLCSEVYRYQVTPRMLCAGYRKGKKDACQGDSGGPLVCKALSGRWFLAGLVSWGLGCGRPNYFGVYTRITGVISWIQQVVT

SEQ ID NO: 608 - protein, 824 aa, Homo sapiens; identical to SEQ ID NO: 606

SEQ ID NO: 609 - protein, 141 aa, Homo sapiens
MPVAEAPQVAGGQGDGGDGEEAEPEGMFKACEDSKRKARGYLRLVPLFVLLALLVLASAGVLLWYFLGYKAEVMVSQVYSGSLRVLNRHFSQDLTRRESSAFRSETAKAQKMLKELITSTRLGTYYNSSSVYSFGEGPLTC

SEQ ID NO: 610 - protein, 71 aa, Homo sapiens
LTLDNRLDSQGVLSTPYFPSYYSPQTHCSWHLTVPSLDYGLALWFDAYALRRQKYDLPCTQGQWTIQNRRV

SEQ ID NO: 611 - protein, 461 aa, Homo sapiens
MLLLFHSKRMPVAEAPQVAGGQGDGGDGEEAEPEGMFKACEDSKRKARGYLRLVPLFVLLALLVLASAGVLLWYFLGYKAEVMVSQVYSGSLRVLNRHFSQDLTRRESSAFRSETAKAQKMLKELITSTRLGTYYNSSSVYSFGEGPLTCFFWFILQIPEHRRLMLSPEVVQALLVEELLSTVNSSAAVPYRAEYEVDPEGLVILEASVKDIAALNSTLGCYRYSYVGQGQVLRLKGPDHLASSCLWHLQGPKDLMLKLRLEWTLAECRDRLAMYDVAGPLEKRLITSVYGCSRQEPVVEVLASGAIMAVVWKKGLHSYYDPFVLSVQPVVFQACEVNLTLDNRLDSQGVLSTPYFPSYYSPQTHCSWHLTVPSLDYGLALWFDAYALRRQKYDLPCTQGQWTIQNRRYHFLSSLWLPFLPPPPSLPSSIVTPSLEAQVPNLRGAARGASRGWGWCQACCP

SEQ ID NO: 612 - protein, 428 aa, Homo sapiens
MLRTLLRRRLFSYPTKYYFMVLVLSLITFSVLRIHQKPEFVSVRHLELAGENPSSDINCTKVLQGDVNEIQKVKLEILTVKFKKRPRWTPDDYINMTSDCSSFIKRRKYIVEPLSKEEAEFPIAYSIVVHHKIEMLDRLLRAIYMPQNFYCIHVDTKSEDSYLAAVMGIASCFSNVFVASRLESVVYASWSRVQADLNCMKDLYAMSANWKYLINLCGMDFPIKTNLEIVRKLKLLMGENNLETERMPSHKEERWKKRYEVVNGKLTNTGTVKMLPPLETPLFSGSAYFVVSREYVGYVLQNEKIQKLMEWAQDTYSPDEYLWATIQRIPEVPGSLPASHKYDLSDMQAVARFVKWQYFEGDVSKGAPYPPCDGVHVRSVCIFGAGDLNWMLRKHHLFANKFDVDVDLFAIQCLDEHLRHKALETLKH

SEQ ID NO: 613 - protein, 428 aa, Homo sapiens; identical to SEQ ID NO: 612

SEQ ID NO: 614 - protein, 428 aa, Homo sapiens; identical to SEQ ID NO: 612

SEQ ID NO: 615 - protein, 219 aa, Homo sapiens
MPRGRKSRRRRNARAAEENRNNRKIQASEASETPMAASVVASTPEDDLSGPEEDPSTPEEASTTPEEASSTAQAQKPSVPRSNFQGTKKSLLMSILALIFIMGNSAKEALVWKVLGKLGMQPGRQHSIFGDPKKIVTEEFVRRGYLIYKPVPRSSPVEYEFFWGPRAHVESSKLKVMHFVARVRNRCSKDWPCNYDWDSDDDAEVEAILNSGARGYSAP

SEQ ID NO: 616 - protein, 85 aa, Homo sapiens
MGPRQGRWWLLLWLPPLATLPVRGEAAAAALSVRRCKALKEKDLIRTSESDCYCYNQNSQVEWKYIWSTMQGDCVLLCGACQEDI

SEQ ID NO: 617 - protein, 417 aa, Homo sapiens
MGPRQGRWWLLLWLPPLATLPVRGEAAAAALSVRRCKALKEKDLIRTSESDCYCYNQNSQVEWKYIWSTMQVKITSPGLFRIVYIAERHNCQYPENILSFIKCVIHNFWIPKESNEITIIINPYRETVCFSVEPVKKIFNYMIHVNRNIMDFKLFLVFVAGVFLFFYARTLSQSPTFYYSSGTVLGVLMTLVFVLLLVKRFIPKYSTFWALMVGCWFASVYIVCQLMEDLKWLWYENRIYVLGYVLIVGFFSFVVCYKHGPLADDRSRSLLMWMLRLLSLVLVYAGVAVPQFAYAAIILLMSSWSLHYPLRACSYMRWKMEQWFTSKELVVKYLTEDEYREQADAETNSALEELRRACRKPDFPSWLVVSRLHTPSKFADFVLGGSHLSPEEISLHEEQYGLGGAFLEEQLFNPSTA

SEQ ID NO: 618 - protein, 138 aa, Homo sapiens
MQVKITSPGLFRIVYIAERHNCQYPENILSFIKCVIHNFWIPKESNEITIIINPYRETVCFSVEPVKKIFNYMIHVNRNIMDFKLFLVFVAGVFLFFYARTLSQSPTFYYSSGTVLGVLMTLVFVLLLVKRFIPKAMS

SEQ ID NO: 619 - protein, 24 aa, Homo sapiens
MQVKITSPGLFRIVYIAERHNCQY

SEQ ID NO: 620 - protein, 35 aa, Homo sapiens
DCYCYNQNSQVEWKYIWSTMQGDCVLLCGACQEDI

SEQ ID NO: 621 - protein, 422 aa, Homo sapiens
MLHLLALFLHCLPLASGDYDICKSWVTTDEGPTWEFYACQPKVMRLKDYVKVKVEPSGITCGDPPERFCSHENPYLCSNECDASNPDLAHPPRLMFDKEEEGLATYWQSITWSRYPSPLEANITLSWNKTVELTDDVVMTFEYGRPTVMVLEKSLDNGRTWQPYQFYAEDCMEAFGMSARRARDMSSSSAHRVLCTEEYSRWAGSKKEKHVRFEVRDRFAIFAGPDLRNMDNLYTRLESAKGLKEFFTLTDLRMRLLRPALGGTYVQRENLYKYFYAISNIEVIGRCKCNLHANLCSMREGSLQCECEHNTTGPDCGKCKKNFRTRSWRAGSYLPLPHGSPNACATAGSFGTLQTPPPGRSPSALRGSRRGLANVKEPAGSRPQISEMLLGCTVTLHQGSVGPHIPPKLSLPDPGGPWLGSQ

SEQ ID NO: 622 - protein, 530 aa, Homo sapiens
MLHLLALFLHCLPLASGDYDICKSWVTTDEGPTWEFYACQPKVMRLKDYVKVKVEPSGITCGDPPERFCSHENPYLCSNECDASNPDLAHPPRLMFDKEEEGLATYWQSITWSRYPSPLEANITLSWNKTVELTDDVVMTFEYGRPTVMVLEKSLDNGRTWQPYQFYAEDCMEAFGMSARRARDMSSSSAHRVLCTEEYSRWAGSKKEKHVRFEVRDRFAIFAGPDLRNMDNLYTRLESAKGLKEFFTLTDLRMRLLRPALGGTYVQRENLYKYFYAISNIEVIGRCKCNLHANLCSMREGSLQCECEHNTTGPDCGKCKKNFRTRSWRAGSYLPLPHGSPNACATAGSFGNCECYGHSNRCSYIDFLNVVTCVSCKHNTRGQHCQHCRLGYYRNGSAELDDENVCIECNCNQIGSVHDRCNETGFCECREGAAGPKCDDCLPTHYWRQGCYPNVCDDDQLLCQNGGTCLQNQRCACPRGYTGVRCEQPRCDPADDDGGLDCDRAPGAAPRPATLLGCLLLLGLAARLGR

SEQ ID NO: 623 - protein, 211 aa, Homo sapiens [sequence omitted]

SEQ ID NO: 624 - protein, 205 aa, Homo sapiens
QRQAAALGSQSPSPAGLRAGAVPPARACLQEAEIPREVIERLARSQIHSIRDLQRLLEIDSVGSEDSLDTSLRAHGVHATKHVPEKRPLPIRRKRSIEEAVPAVCKTRTVIYEIPRSQVDPTSANFLIWPPCVEVKRCTGCCNTSSVKCQPSRVHHRSVKVAKVEYVRKKPKLKEVQVRLEEHLECACATTSLNPDYREEDTDVR

SEQ ID NO: 625 - protein, 196 aa, Homo sapiens
MRTLACLLLLGCGYLAHVLAEEAEIPREVIERLARSQIHSIRDLQRLLEIDSVGSEDSLDTSLRAHGVHATKHVPEKRPLPIRRKRSIEEAVPAVCKTRTVIYEIPRSQVDPTSANFLIWPPCVEVKRCTGCCNTSSVKCQPSRVHHRSVKVAKVEYVRKKPKLKEVQVRLEEHLECACATTSLNPDYREEDTDVR

SEQ ID NO: 626 - protein, 86 aa, Homo sapiens
MRTLACLLLLGCGYLAHVLAEEAEIPREVIERLARSQIHSIRDLQRLLEIDSVGSEDSLDTSLRAHGVHATKHVPEKRPLPIRRKR

SEQ ID NO: 627 - protein, 273 aa, Homo sapiens
MIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWLLHIFIPFCIIAFIFIATVIALRKQLCQKLYSSKDTTKRPVTTTKREVNSAI

SEQ ID NO: 628 - protein, 423 aa, Homo sapiens
MDRGQPSLEPAAAAPRASGRCVIAPVRAVLRLRRRVCVLRKRRLLQPGGGPDVGTGAPRPGCSPRAPRADLDQPKFFTFDSPAELPSRTPRKKRRRSRLVLYPETSRKYRPRVEHRSRAQRCLLLLVAIVGFQVLNAIENLDDNAQRYDLDGLEKALQRAVFGQPAAVSRIVALMRDYLATHVHSRPLLLALHGPSGVGKSHVGRLLARHFRSVLEDSALVLQYHARHHCPEARAAQDCREELARRVADVVARAEAEEKTPLLVLDDVELMPRPLLDELHGFLQPQRSHHFHNAIYVLLSGAGGAEVTRFVLQNASRALPLRPDGFRSAEAAAAQAEEDLRASLLAVLSREHPLWQAAAIVPFLLLDKRDVVSCFRDEMAGEGFFPDQARAENLAAQLSFYRVAGREFAVTGCKQVVATVNLL

SEQ ID NO: 629 - protein, 69 aa, Homo sapiens
XISEAEKAKQKIAPVFFREEYMLNNAVGSCQKPYVALIHGITMGGDCSLMWVEVISCHDSKENLVTSLH

SEQ ID NO: 630 - protein, 52 aa, Homo sapiens
DVGGGYFLPRLQGKLGYFLALTGFRLKGRDVYRAGIATHFVDSEKNGDDTIT

SEQ ID NO: 631 - protein, 793 aa, Homo sapiens
MLRGGRRGQLGWHSWAAGPGSLLAWLILASAGAAPCPDACCPHGSSGLRCTRDGALDSLHHLPGAENLTELYIENQQHLQHLELRDLRGLGELRNLTIVKSGLRFVAPDAFHFTPRLSRLNLSFNALESLSWKTVQGLSLQELVLSGNPLHCSCALRWLQRWEEEGLGGVPEQKLQCHGQGPLAHMPNASCGVPTLKVQVPNASVDVGDDVLLRCQVEGRGLEQAGWILTELEQSATVMKSGGLPSLGLTLANVTSDLNRKNVTCWAENDVGRAEVSVQVNVSFPASVQLHTAVEMHHWCIPFSVDGQPAPSLRWLFNGSVLNETSFIFTEFLEPAANETVRHGCLRLNQPTHVNNGNYTLLAANPFGQASASIMAAFMDNPFEFNPEDPIPDTNSTSGDPVEKKDETPFGVSVAVGLAVFACLFLSTLLLVLNKCGRRNKFGINRPAVLAPEDGLAMSLHFMTLGGSSLSPTEGKGSGLQGHIIENPQYFSDASPSGVHHIKRRDIVLKWELGEGAFGKVFLAECHNLLPEQDKMLVAVKALKEASESARQDFQREAELLTMLQHQHIVRFFGVCTEGRPLLMVFEYMRHGDLNRFLRSHGPDAKLLAGGEDVAPGPLGLGQLLAVASQVAAGMVYLAGLHFVHRDLATRNCLVGQGLVVKIGDFGMSRDIYSTDYYRVGGRTMLPIRWMPPESILYRKFTTESDVWSFGVVLWEIFTYGKQPWYQLSNTEAIDCITQGRELERPRACPPEVYAIMRGCWQREPQQRHSIKDVHARLQALAQAPPVYLDVLG

SEQ ID NO: 632 - protein, 790 aa, Homo sapiens
MLRGGRRGQLGWHSWAAGPGSLLAWLILASAGAAPCPDACCPHGSSGLRCTRDGALDSLHHLPGAENLTELYIENQQHLQHLELRDLRGLGELRNLTIVKSGLRFVAPDAFHFTPRLSRLNLSFNALESLSWKTVQGLSLQELVLSGNPLHCSCALRWLQRWEEEGLGGVPEQKLQCHGQGPLAHMPNASCGVPTLKVQVPNASVDVGDDVLLRCQVEGRGLEQAGWILTELEQSATVMKSGGLPSLGLTLANVTSDLNRKNVTCWAENDVGRAEVSVQVNVSFPASVQLHTAVEMHHWCIPFSVDGQPAPSLRWLFNGSVLNETSFIFTEFLEPAANETVRHGCLRLNQPTHVNNGNYTLLAANPFGQASASIMAAFMDNPFEFNPEDPIPDTNSTSGDPVEKKDETPFGVSVAVGLAVFACLFLSTLLLVLNKCGRRNKFGINRPAVLAPEDGLAMSLHFMTLGGSSLSPTEGKGSGLQGHIIENPQYFSDACVHHIKRRDIVLKWELGEGAFGKVFLAECHNLLPEQDKMLVAVKALKEASESARQDFQREAELLTMLQHQHIVRFFGVCTEGRPLLMVFEYMRHGDLNRFLRSHGPDAKLLAGGEDVAPGPLGLGQLLAVASQVAAGMVYLAGLHFVHRDLATRNCLVGQGLVVKIGDFGMSRDIYSTDYYRVGGRTMLPIRWMPPESILYRKFTTESDVWSFGVVLWEIFTYGKQPWYQLSNTEAIDCITQGRELERPRACPPEVYAIMRGCWQREPQQRHSIKDVHARLQALAQAPPVYLDVLG

SEQ ID NO: 633 - protein, 760 aa, Homo sapiens
MKEAALICLAPSVPPILTVKSWDTMQLRAARSRCTNLLAASYIENQQHLQHLELRDLRGLGELRNLTIVKSGLRFVAPDAFHFTPRLSRLNLSFNALESLSWKTVQGLSLQELVLSGNPLHCSCALRWLQRWEEEGLGGVPEQKLQCHGQGPLAHMPNASCGVPTLKVQVPNASVDVGDDVLLRCQVEGRGLEQAGWILTELEQSATVMKSGGLPSLGLTLANVTSDLNRKNVTCWAENDVGRAEVSVQVNVSFPASVQLHTAVEMHHWCIPFSVDGQPAPSLRWLFNGSVLNETSFIFTEFLEPAANETVRHGCLRLNQPTHVNNGNYTLLAANPFGQASASIMAAFMDNPFEFNPEDPIPDTNSTSGDPVEKKDETPFGVSVAVGLAVFACLFLSTLLLVLNKCGRRNKFGINRPAVLAPEDGLAMSLHFMTLGGSSLSPTEGKGSGLQGHIIENPQYFSDACVHHIKRRDIVLKWELGEGAFGKVFLAECHNLLPEQDKMLVAVKALKEASESARQDFQREAELLTMLQHQHIVRFFGVCTEGRPLLMVFEYMRHGDLNRFLRSHGPDAKLLAGGEDVAPGPLGLGQLLAVASQVAAGMVYLAGLHFVHRDLATRNCLVGQGLVVKIGDFGMSRDIYSTDYYRVGGRTMLPIRWMPPESILYRKFTTESDVWSFGVVLWEIFTYGKQPWYQLSNTEAIDCITQGRELERPRACPPEVYAIMRGCWQREPQQRHSIKDVHARLQALAQAPPVYLDVLG

SEQ ID NO: 634 - protein, 388 aa, Homo sapiens
MKEAALICLAPSVPPILTVKSWDTMQLRAARSRCTNLLAASYIENQQHLQHLELRDLRGLGELRNLTIVKSGLRFVAPDAFHFTPRLSRLNLSFNALESLSWKTVQGLSLQELVLSGNPLHCSCALRWLQRWEEEGLGGVPEQKLQCHGQGPLAHMPNASCGVPTLKVQVPNASVDVGDDVLLRCQVEGRGLEQAGWILTELEQSATVMSRPVCSCTRRWRCTTGASPSLWMGSRHRLCAGSSMAPCSMRPASSSLSSWSRQPMRPCGTGVCASTSPPTSTTATTRCWLPTPSARPPPPSWLPSWTTLSSSTPRTPSLTLTAHLETRWRRRTKHLLGSRWLWAWPSLPASSFLRCSLCSTNVDGETSLGSTARLCWLQRMGWPCPCIS

SEQ ID NO: 635 - protein, 796 aa, Homo sapiens
MLRGGRRGQLGWHSWAAGPGSLLAWLILASAGAAPCPDACCPHGSSGLRCTRDGALDSLHHLPGAENLTELYIENQQHLQHLELRDLRGLGELRNLTIVKSGLRFVAPDAFHFTPRLSRLNLSFNALESLSWKTVQGLSLQELVLSGNPLHCSCALRWLQRWEEEGLGGVPEQKLQCHGQGPLAHMPNASCGVPTLKVQVPNASVDVGDDVLLRCQVEGRGLEQAGWILTELEQSATVMKSGGLPSLGLTLANVTSDLNRKNVTCWAENDVGRAEVSVQVNVSFPASVQLHTAVEMHHWCIPFSVDGQPAPSLRWLFNGSVLNETSFIFTEFLEPAANETVRHGCLRLNQPTHVNNGNYTLLAANPFGQASASIMAAFMDNPFEFNPEDPIPVSFSPVDTNSTSGDPVEKKDETPFGVSVAVGLAVFACLFLSTLLLVLNKCGRRNKFGINRPAVLAPEDGLAMSLHFMTLGGSSLSPTEGKGSGLQGHIIENPQYFSDACVHHIKRRDIVLKWELGEGAFGKVFLAECHNLLPEQDKMLVAVKALKEASESARQDFQREAELLTMLQHQHIVRFFGVCTEGRPLLMVFEYMRHGDLNRFLRSHGPDAKLLAGGEDVAPGPLGLGQLLAVASQVAAGMVYLAGLHFVHRDLATRNCLVGQGLVVKIGDFGMSRDIYSTDYYRVGGRTMLPIRWMPPESILYRKFTTESDVWSFGVVLWEIFTYGKQPWYQLSNTEAIDCITQGRELERPRACPPEVYAIMRGCWQREPQQRHSIKDVHARLQALAQAPPVYLDVLG

SEQ ID NO: 636 - protein, 131 aa, Homo sapiens
MSKRYLQKATKGKLLIIIFIVTLWGKVVSSANHHKAHHVKTGTCEVVALHRCCNKNKIEERSQTVKCSCFPGQVAGTTRAAPSCVDASIVEQKWWCHMQPCLEGEECKVLPDRKGWSCSSGNKVKTTRVTH

SEQ ID NO: 637 - protein, 87 aa, Homo sapiens
MMSKRYLQKATKGKLLIIIFIVTLWGKVVSSANHHKAHHVKTGTCEVVALHRCCNKNKIEERSQTVKCSCFPGQVAGTTRAAPSCVD

SEQ ID NO: 638 - protein, 131 aa, Homo sapiens
MSKRYLQKATKGKLLIIIFIVTLWGKVVSSANHHKAHHVKTGTCEVVALHRCCNKNKIEERSQTVKCSCFPGQVAGTTRAAPSCVDASIVEQKWWCHMQPCLEGEECKVLPDRKGWSCSSGNKVKTTRANV

SEQ ID NO: 639 - protein, 101 aa, Homo sapiens
MVTPITRMSKRYLQKATKGKLLIIIFIVTLWGKVVSSANHHKAHHVKTGTCEVVALHRCCNKNKIEERSQTVKCSCFPGQVAGTTRAAPSCVDASIVEQKW

SEQ ID NO: 640 - protein, 34 aa, Homo sapiens
MQPCLEGEECKVLPDRKGWSCSSGNKVKTTRVTH

SEQ ID NO: 641 - protein, 49 aa, Homo sapiens
MSKRYLQKATKGKLLIIIFIVTLWGKVVSSANHHKGNPLTQEKSSDPQG

SEQ ID NO: 642 - protein, 131 aa, Homo sapiens; identical to SEQ ID NO: 636

SEQ ID NO: 643 - protein, 110 aa, Homo sapiens
MMSKRYLQKATKGKLLIIIFIVTLWGKVVSSANHHKAHHVKTGTCEVVALHRCCNKNKIEERSQTVKCSCFPGQVAGTTRAAPSCVDASIVEQKWWCHMQPCLEGEECKV

SEQ ID NO: 644 - protein, 2229 aa, Homo sapiens
MFPGCPRLWVLVVLGTSWVGWGSQGTEAAQLRQFYVAAQGISWSYRPEPTNSSLNLSVTSFKKIVYREYEPYFKKEKPQSTISGLLGPTLYAEVGDIIKVHFKNKADKPLSIHPQGIRYSKLSEGASYLDHTFPAEKMDDAVAPGREYTYEWSISEDSGPTHDDPPCLTHIYYSHENLIEDFNSGLIGPLLICKKGTLTEGGTQKTFDKQIVLLFAVFDESKSWSQSSSLMYTVNGYVNGTMPDITVCAHDHISWHLLGMSSGPELFSIHFNGQVLEQNHHKVSAITLVSATSTTANMTVGPEGKWIISSLTPKHLQAGMQAYIDIKNCPKKTRNLKKITREQRRHMKRWEYFIAAEEVIWDYAPVIPANMDKKYRSQHLDNFSNQIGKHYKKVMYTQYEDESFTKHTVNPNMKEDGILGPIIRAQVRDTLKIVFKNMASRPYSIYPHGVTFSPYEDEVNSSFTSGRNNTMIRAVQPGETYTYKWNILEFDEPTENDAQCLTRPYYSDVDIMRDIASGLIGLLLICKSRSLDRRGIQRAADIEQQAVFAVFDENKSWYLEDNINKFCENPDEVKRDDPKFYESNIMSNFTLSAINGYVPESITTLGFCFDDTVQWHFCSVGTQNEILTIHFTGHSFIYGKRHEDTLTLFPMRGESVTVTMDNVGTWMLTSMNSSPRSKKLRLKFRDVKCIPDDDEDSYEIFEPPESTVMATRKMHDRLEPEDEESDADYDYQNRLAAALGIRSFRNSSLNQEEEEFNLTALALENGTEFVSSNTDIIVGSNYSSPSNISKFTVNNLAEPQKAPSHQQATTAGSPLRHLIGKNSVLNSSTAEHSSPYSEDPIEDPLQPDVTGIRLLSLGAGEFKSQEHAKHKGPKVERDQAAKHRFSWMKLLAHKVGRHLSQDTGSPSGMRPWEDLPSQDTGSPSRMRPWKDPPSDLLLLKQSNSSKILVGRWHLASEKGSYEIIQDTDEDTAVNNWLISPQNASRAWGESTPLANKPGKQSGHPKFPRVRHKSLQVRQDGGKSRLKKSQFLIKTRKKKKEKHTHHAPLSPRTFHPLRSEAYNTFSERRLKHSLVLHKSNETSLPTDLNQTLPSMDFGWIASLPDHNQNSSNDTGQASCPPGLYQTVPPEEHYQTFPIQDPDQMHSTSDPSHRSSSPELSEMLEYDRSHKSFPTDISQMSPSSEHEVWQTVISPDLSQVTLSPELSQTNLSPDLSHTTLSPELIQRNLSPALGQMPISPDLSHTTLSPDLSHTTLSLDLSQTNLSPELSQTNLSPALGQMPLSPDLSHTTLSLDFSQTNLSPELSHMTLSPELSQTNLSPALGQMPISPDLSHTTLSLDFSQTNLSPELSQTNLSPALGQMPLSPDPSHTTLSLDLSQTNLSPELSQTNLSPDLSEMPLFADLSQIPLTPDLDQMTLSPDLGETDLSPNFGQMSLSPDLSQVTLSPDISDTTLLPDLSQISPPPDLDQIFYPSESSQSLLLQEFNESFPYPDLGQMPSPSSPTLNDTFLSKEFNPLVIVGLSKDGTDYIEIIPKEEVQSSEDDYAEIDYVPYDDPYKTDVRTNINSSRDPDNIAAWYLRSNNGNRRNYYIAAEEISWDYSEFVQRETDIEDSDDIPEDTTYKKVVFRKYLDSTFTKRDPRGEYEEHLGILGPIIRAEVDDVIQVRFKNLASRPYSLHAHGLSYEKSSEGKTYEDDSPEWFKEDNAVQPNSSYTYVWHATERSGPESPGSACRAWAYYSAVNPEKDIHSGLIGPLLICQKGILHKDSNMPMDMREFVLLFMTFDEKKSWYYEKKSRSSWRLTSSEMKKSHEFHAINGMIYSLPGLKMYEQEWVRLHLLNIGGSQDIHVVHFHGQTLLENGNKQHQLGVWPLLPGSFKTLEMKASKPGWWLLNTEVGENQRAGMQTPFLIMDRDCRMPMGLSTGIISDSQIKASEFLGYWEPRLARLNNGGSYNAWSVEKLAAEFASKPWIQVDMQKEVIITGIQTQGAKHYLKSCYTTEFYVAYSSNQINWQIFKGNSTRNVMYFNGNSDASTIKENQFDPPIVARYIRISPTRAYNRPTLRLELQGCEVNGCSTPLGMENGKIENKQITASSFKKSWWGDYWEPFRARLNAQGRVNAWQAKANNNKQWLEIDLLKIKKITAIITQGCKSLSSEMYVKSYTIHYSEQGVEWKPYRLKSSMVDKIFEGNTNTKGHVKNFFNPPIISRFIRVIPKTWNQSIALRLELFGCDIY

SEQ ID NO: 645 - protein, 2224 aa, Homo sapiens
MFPGCPRLWVLVVLGTSWVGWGSQGTEAAQLRQFYVAAQGISWSYRPEPTNSSLNLSVTSFKKIVYREYEPYFKKEKPQSTISGLLGPTLYAEVGDIIKVHFKNKADKPLSIHPQGIRYSKLSEGASYLDHTFPAEKMDDAVAPGREYTYEWSISEDSGPTHDDPPCLTHIYYSHENLIEDFNSGLIGPLLICKKGTLTEGGTQKTFDKQIVLLFAVFDESKSWSQSSSLMYTVNGYVNGTMPDITVCAHDHISWHLLGMSSGPELFSIHFNGQVLEQNHHKVSAITLVSATSTTANMTVGPEGKWIISSLTPKHLQAGMQAYIDIKNCPKKTRNLKKITREQRRHMKRWEYFIAAEEVIWDYAPVIPANMDKKYRSQHLDNFSNQIGKHYKKVMYTQYEDESFTKHTVNPNMKEDGILGPIIRAQVRDTLKIVFKNMASRPYSIYPHGVTFSPYEDEVNSSFTSGRNNTMIRAVQPGETYTYKWNILEFDEPTENDAQCLTRPYYSDVDIMRDIASGLIGLLLICKSRSLDRRGIQRAADIEQQAVFAVFDENKSWYLEDNINKFCENPDEVKRDDPKFYESNIMSTINGYVPESITTLGFCFDDTVQWHFCSVGTQNEILTIHFTGHSFIYGKRHEDTLTLFPMRGESVTVTMDNVGTWMLTSMNSSPRSKKLRLKFRDVKCIPDDDEDSYEIFEPPESTVMATRKMHDRLEPEDEESDADYDYQNRLAAALGIRSFRNSSLNQEEEEFNLTALALENGTEFVSSNTDIIVGSNYSSPSNISKFTVNNLAEPQKAPSHQQATTAGSPLRHLIGKNSVLNSSTAEHSSPYSEDPIEDPLQPDVTGIRLLSLGAGEFKSQEHAKHKGPKVERDQAAKHRFSWMKLLAHKVGRHLSQDTGSPSGMRPWEDLPSQDTGSPSRMRPWKDPPSDLLLLKQSNSSKILVGRWHLASEKGSYEIIQDTDEDTAVNNWLISPQNASRAWGESTPLANKPGKQSGHPKFPRVRHKSLQVRQDGGKSRLKKSQFLIKTRKKKKEKHTHHAPLSPRTFHPLRSEAYNTFSERRLKHSLVLHKSNETSLPTDLNQTLPSMDFGWIASLPDHNQNSSNDTGQASCPPGLYQTVPPEEHYQTFPIQDPDQMHSTSDPSHRSSSPELSEMLEYDRSHKSFPTDISQMSPSSEHEVWQTVISPDLSQVTLSPELSQTNLSPDLSHTTLSPELIQRNLSPALGQMPISPDLSHTTLSPDLSHTTLSLDLSQTNLSPELSQTNLSPALGQMPLSPDLSHTTLSLDFSQTNLSPELSHMTLSPELSQTNLSPALGQMPISPDLSHTTLSLDFSQTNLSPELSQTNLSPALGQMPLSPDPSHTTLSLDLSQTNLSPELSQTNLSPDLSEMPLFADLSQIPLTPDLDQMTLSPDLGETDLSPNFGQMSLSPDLSQVTLSPDISDTTLLPDLSQISPPPDLDQIFYPSESSQSLLLQEFNESFPYPDLGQMPSPSSPTLNDTFLSKEFNPLVIVGLSKDGTDYIEIIPKEEVQSSEDDYAEIDYVPYDDPYKTDVRTNINSSRDPDNIAAWYLRSNNGNRRNYYIAAEEISWDYSEFVQRETDIEDSDDIPEDTTYKKVVFRKYLDSTFTKRDPRGEYEEHLGILGPIIRAEVDDVIQVRFKNLASRPYSLHAHGLSYEKSSEGKTYEDDSPEWFKEDNAVQPNSSYTYVWHATERSGPESPGSACRAWAYYSAVNPEKDIHSGLIGPLLICQKGILHKDSNMPMDMREFVLLFMTFDEKKSWYYEKKSRSSWRLTSSEMKKSHEFHAINGMIYSLPGLKMYEQEWVRLHLLNIGGSQDIHVVHFHGQTLLENGNKQHQLGVWPLLPGSFKTLEMKASKPGWWLLNTEVGENQRAGMQTPFLIMDRDCRMPMGLSTGIISDSQIKASEFLGYWEPRLARLNNGGSYNAWSVEKLAAEFASKPWIQVDMQKEVIITGIQTQGAKHYLKSCYTTEFYVAYSSNQINWQIFKGNSTRNVMYFNGNSDASTIKENQFDPPIVARYIRISPTRAYNRPTLRLELQGCEVNGCSTPLGMENGKIENKQITASSFKKSWWGDYWEPFRARLNAQGRVNAWQAKANNNKQWLEIDLLKIKKITAIITQGCKSLSSEMYVKSYTIHYSEQGVEWKPYRLKSSMVDKIFEGNTNTKGHVKNFFNPPIISRFIRVIPKTWNQSIALRLELFGCDIY

SEQ ID NO: 646 - protein, 553 aa, Homo sapiens
MAASKKAVLGPLVGAVDQGTSSTRFLVFNSKTAELLSHHQVEIKQEFPREGWVEQDPKEILHSVYECIEKTCEKLGQLNIDISNIKAIGVSNQRETTVVWDKITGEPLYNAVVWLDLRTQSTVESLSKRIPGNNNFVKSKTGLPLSTYFSAVKLRWLLDNVRKVQKAVEEKRALFGTIDSWLIWSLTGGVNGGVHCTDVTNASRTMLFNIHSLEWDKQLCEFFGIPMEILPNVRSSSEIYGLMKAGALEGVPISGCLGDQSAALVGQMCFQIGQAKNTYGTGCFLLCNTGHKCVFSDHGLLTTVAYKLGRDKPVYYALEGSVAIAGAVIRWLRDNLGIIKTSEEIEKLAKEVGTSYGCYFVPAFSGLYAPYWEPSARGIICGLTQFTNKCHIAFAALEAVCFQTREILDAMNRDCGIPLSHLQVDGGMTSNKILMQLQADILYIPVVKPSMPETTALGAAMAAGAAEGVGVWSLEPEDLSAVTMERFEPQINAEESEIRYSTWKKAVMKSMGWVTTQSPESGDPSIFCSLPLGFFIVSSMVMLIGARYISGIP

SEQ ID NO: 647 - protein, 559 aa, Homo sapiens
MAASKKAVLGPLVGAVDQGTSSTRFLVFNSKTAELLSHHQVEIKQEFPREGWVEQDPKEILHSVYECIEKTCEKLGQLNIDISNIKAIGVSNQRETTVVWDKITGEPLYNAVVWLDLRTQSTVESLSKRIPGNNNFVKSKTGLPLSTYFSAVKLRWLLDNVRKVQKAVEEKRALFGTIDSWLIWSLTGGVNGGVHCTDVTNASRTMLFNIHSLEWDKQLCEFFGIPMEILPNVRSSSEIYGLMKISHSVKAGALEGVPISGCLGDQSAALVGQMCFQIGQAKNTYGTGCFLLCNTGHKCVFSDHGLLTTVAYKLGRDKPVYYALEGSVAIAGAVIRWLRDNLGIIKTSEEIEKLAKEVGTSYGCYFVPAFSGLYAPYWEPSARGIICGLTQFTNKCHIAFAALEAVCFQTREILDAMNRDCGIPLSHLQVDGGMTSNKILMQLQADILYIPVVKPSMPETTALGAAMAAGAAEGVGVWSLEPEDLSAVTMERFEPQINAEESEIRYSTWKKAVMKSMGWVTTQSPESGDPSIFCSLPLGFFIVSSMVMLIGARYISGIP

SEQ ID NO: 648 - protein, 41 aa, Homo sapiens
SARVAKFRCCLIPDSDSITCIPLKPAFSGMAPDVAYSTPTS

SEQ ID NO: 649 - protein, 57 aa, Homo sapiens
MRLHYRYKEASGYFHTLRAVMRNPEEDGKDTLQCIAEMLQITKQAMGSDLPIIEKKL

SEQ ID NO: 650 - protein, 183 aa, Homo sapiens
MPRSHRPPPTAQRAATAAAAEPKSEGVVAMTFKIFLLFAGLMVKVPVGLYFSCKLLLFQSLMLMSPEDSGFYATIVAVVGLHVQQFSECTLIDATHRDVDVLLLLSNSAYYVAYYDDEVDKVNQYQRLSLENLEKIEIGPEPTLFGKPKFSCMRLHYRYKEASGYFHTLRAVMRNPEEDGKDT

SEQ ID NO: 651 - protein, 145 aa, Homo sapiens
MSAVLLLALLGFILPLPGVQALLCQFGTVQHVWKVSDLPRQWTPKNTSCDSGLGCQDTLMLIESGPQVSLVLSKGCTEAKDQEPRVTEHRMGPGLSLISYTFVCRQEDFCNNLVNSLPLWAPQPPADPMMGAAEGPFGKAEQVDS

SEQ ID NO: 652 - protein, 139 aa, Homo sapiens
MSAVLLLALLGFILPLPGVQALLCQFGTVQHVWKVSDLPRQWTPKNTSCDSGLGCQDTLMLIESGPQVSLVLSKGCTEAKDQEPRVTEHRMGPGLSLISYTFVCRQEDFCNNLVNSLPLWAPQPPAGACGRVGRRGRGC

SEQ ID NO: 653 - protein, 437 aa, Homo sapiens
MSAVLLLALLGFILPLPGVQALLCQFGTVQHVWKVSDLPRQWTPKNTSCDSGLGCQDTLMLIESGPQVSLVLSKGCTEAKDQEPRVTEHRMGPGLSLISYTFVCRQEDFCNNLVNSLPLWAPQPPADPGSLRCPVCLSMEGCLEGTTEEICPKGTTHCYDGLLRLRGGGIFSNLRVQGCMPQPVCNLLNGTQEIGPVGMTENCDMKDFLTCHRGTTIMTHGNLAQEPTDWTTSNTEMCEVGQVCQETLLLLDVGLTSTLVGTKGCSTVGAQNSQKTTIHSAPPGVLVASYTHFCSSDLCNSASSSSVLLNSLPPQAAPVPGDRQCPTCVQPLGTCSSGSPRMTCPRGATHCYDGYIHLSGGGLSTKMSIQGCVAQPSSFLLNHTRQIGIFSAREKRDVQPPASQHEGGGAEGLESLTWGVGLALAPALWWGVVCPSC

SEQ ID NO: 654 - protein, 131 aa, Homo sapiens
MAGVKALVALSFSGAIGLTFLMLGCALEDYGVYWPLFVLIFHAISPIPHFIAKRVTYDSDATSSACRELAYFFTTGIVVSAFGFPVILARVAVIKWGACGLVLAGNAVIFLTIQGFFLIFGRGDDFSWEQW

SEQ ID NO: 655 - protein, 140 aa, Homo sapiens
MRDGKGFLQGFLYFALVALSFSGAIGLTFLMLGCALEDYGVYWPLFVLIFHAISPIPHFIAKRVTYDSDATSSACRELAYFFTTGIVVSAFGFPVILARVAVIKWGACGLVLAGNAVIFLTIQGFFLIFGRGDDFSWEQW

SEQ ID NO: 656 - protein, 140 aa, Homo sapiens
MQFGELLAAVRKAQANVMLFLEEKEQAALSQANGIKAHLEYRSAEMEKSKQELERMAAISNTVQFLEMRMTSRLTRTQHTSISGCRRRTARSPTPRPGSIPTRTSPAGSCTGGRCCPSRVCTCTGTILRWRSSGQAPMLA

SEQ ID NO: 657 - protein, 205 aa, Homo sapiens
MTPPERLFLPRVCGTTLHLLLLGLLLVLLPGAQGLPGVGLTPSAAQTARQHPKMHLAHSTLKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFAL

SEQ ID NO: 658 - protein, 205 aa, Homo sapiens; identical to SEQ ID NO: 657

SEQ ID NO: 659 - protein, 1015 aa, Homo sapiens
MLTALAPPALPGIPRQLPTAPARRQDSSGSSGSYYTAPGSPEPPDVGPDAKGPANWPWVAPGRGAGAQPRLSVSAQNSRQRHGPGSGFPRGPGSGPRPPQPQLRTLPSGEMEVIFGVGPLFGCSGADDREAQQQFTEPAFISPLPPGPASPAAVPRQSQVPDGGSRWATYLELRPRGPSPAAPAQFECVEVALEEGAAPARPRTVPKRQIELRPRPQSPPRAAGAPRPRLLLRTGSLDESLGPLQAAAGFVQTALARKLSPEAPAPSSATFGSTGRSEPETRETARSTHVVLEKAKSRPLRVRDNSAPAKAPRPWPSLRERAIRRDKPAPGTEPLGPVSSSIFLQSEEKIQEARKTRFPREAPDRTVQRARSPPFECRIPSEVPSRAVRPRSPSPPRQTPNGAVRGPRCPSPQNLSPWDRTTRRVSSPLFPEASSEWENQNPAVEETVSRRSPSPPILSQWNQCVAGERSPSLEAPSLWEIPHSAVADAVEPRSSPSPPAFFPWEAPDRPIGTWGPSPQETWDPMGPGSSIAFTQEAQNGLTQEELAPPTPSAPGTPEPTEMQSPSTREISDLAFGGSQQSPEVAAPEPPGSHPVGTLDADKCPEVLGPGEAASGRPRMAIPRPRDVRKLVKTTYAPGFPAGAQGSGLPAPPADPCGEEGGESKTQEPPALGPPAPAHYTSVFIKDFLPVVPHPYEPPEPSFDTVARDASQPNGVLRRRAENSTAKPFKRTEIRLPGALALGRRPEVTSRVRARGPGGENRDVEAQRLVPDGDGRTSPLGGARSSSQRSPVGPAGVRSPRPGSPQMQASPSPGIAPKPKTPPTAPEPAAAVQAPLPREPLALAGRTAPAQPRAASAPPTDRSPQSPSQGARRQPGAAPLGKVLVDPESGRYYFVEAPRQPRLRVLFDPESGQYVEVLLPPSSPGPPHRVYTPLALGLGLYPPAYGPIPSLSLPPSPGPQALGSPQLPWVSEAGPLDGTYYLPVSGTPNPAPPLLLCAPPSSSGPTQPGKGSLFPL

SEQ ID NO: 660 - protein, 418 aa, Homo sapiens
MDMLHPSSVSTTSEPENASSAWPPDATLGNVSAGPSPAGLAVSGVLIPLVYLVVCVVGLLGNSLVIYVVLRHTASPSVTNVYILNLALADELFMLGLPFLAAQNALSYWPFGSLMCRLVMAVDGINQFTSIFCLTVMSVDRYLAVVHPTRSARWRTAPVARTVSAAVWVASAVVVLPVVVFSGVPRGMSTCHMQWPEPAAAWRAGFIIYTAALGFFGPLLVICLCYLLIVVKVRSAGRRVWAPSCQRRRRSERRVTRMVVAVVALFVLCWMPFYVLNIVNVVCPLPEEPAFFGLYFLVVALPYANSCANPILYGFLSYRFKQGFRRVLLRPSRRVRSQEPTVGPPEKTEEEDEEEEDGEESREGGKGKEMNGRVSQITQPGTSGQERPPSRVASKEQQLLPQEASTGEKSSTMRISYL

SEQ ID NO: 661 - protein, 418 aa, Homo sapiens; identical to SEQ ID NO: 660

SEQ ID NO: 662 - protein, 526 aa, Homo sapiens
MGHLSAPLHRVRVPWQGLLLTASLLTFWNPPTTAQLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTELSPVVAKPQIKASKTTVTGDKDSVNLTCSTNDTGISIRWFFKNQSLPSSERMKLSQGNTTLSINPVKREDAGTYWCEVFNPISKNQSDPIMLNVNYNALPQENGLSPGAIAGIVIGVVALVALIAVALACFLHFGKTGRASDQRDLTEHKPSVSNHTQDHSNDPPNKMNEVTYSTLNFEAQQPTQPTSASPSLTATEIIYSEVKKQ

SEQ ID NO: 663 - protein, 430 aa, Homo sapiens
MGHLSAPLHRVRVPWQGLLLTASLLTFWNPPTTAQLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTDNALPQENGLSPGAIAGIVIGVVALVALIAVALACFLHFGKTGRASDQRDLTEHKPSVSNHTQDHSNDPPNKMNEVTYSTLNFEAQQPTQPTSASPSLTATEIIYSEVKKQ

SEQ ID NO: 664 - protein, 461 aa, Homo sapiens
MGHLSAPLHRVRVPWQGLLLTASLLTFWNPPTTAQLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTERQNLTMLPRLDSNSWAQAILPSVSQSAEITDNALPQENGLSPGAIAGIVIGVVALVALIAVALACFLHFGKTGRASDQRDLTEHKPSVSNHTQDHSNDPPNKMNEVTYSTLNFEAQQPTQPTSASPSLTATEIIYSEVKKQ

SEQ ID NO: 665 - protein, 464 aa, Homo sapiens
MGHLSAPLHRVRVPWQGLLLTASLLTFWNPPTTAQLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTELSPVVAKPQIKASKTTVTGDKDSVNLTCSTNDTGISIRWFFKNQSLPSSERMKLSQGNTTLSINPVKREDAGTYWCEVFNPISKNQSDPIMLNVNYNALPQENGLSPGAIAGIVIGVVALVALIAVALACFLHFGKTGSSGPLQ

SEQ ID NO: 666 - protein, 368 aa, Homo sapiens
MGHLSAPLHRVRVPWQGLLLTASLLTFWNPPTTAQLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTDNALPQENGLSPGAIAGIVIGVVALVALIAVALACFLHFGKTGSSGPLQ

SEQ ID NO: 667 - protein, 94 aa, Homo sapiens
MPFNVAEGKEVLLLVHNLPQQLFGYSWYKGERVDGNRQIVGYAIGTQQATPGPANSGRETIYPNASLLIQNVTQNDTGFYTLQVIKSDLVNEEA

SEQ ID NO: 668 - protein, 399 aa, Homo sapiens
MGHLSAPLHRVRVPWQGLLLTASLLTFWNPPTTAQLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTEMAFHHVAKAGLKLLSSSNPPASTSQSAKITDNALPQENGLSPGAIAGIVIGVVALVALIAVALACFLHFGKTGSSGPLQ

SEQ ID NO: 669 - protein, 99 aa, Homo sapiens
MPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFH

SEQ ID NO: 670 - protein, 174 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIAKEKKPSYNRGLCENAPNRARM

SEQ ID NO: 671 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 672 - protein, 137 aa, Homo sapiens
MHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIAKEKKPSYNRGLCENAPNRARM

SEQ ID NO: 673 - protein, 79 aa, Homo sapiens
MACLGFQRHKAQLNLATRTWPCTLLFFLLFIPVFCKAMHVAQPAVVLASSRGIASFVSKEKKPSYNRGLCENAPNRARM

SEQ ID NO: 674 - protein, 244 aa, Homo sapiens
MRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVALTRKKKALRIHSVEGDLRRKSAGQEEWSPSAPSPPGSCVQAEAAPAGLCGEQRGEDCAELHDYFNVLSYRSLGNCSFFTETG

SEQ ID NO: 675 - protein, 169 aa, Homo sapiens
MMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVALTRKKKALRIHSVEGDLRRKSAGQEEWS

SEQ ID NO: 676 - protein, 311 aa, Homo sapiens
MAFLVAHPMQFVYLLTTLCVFNMVFAKLGFSETVFSQRLSFTVLSAVGYFQWQKRPHLLPVGPLGRSMRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVALTRKKKALRIHSVEGDLRRKSAGQEEWSPSAPSPPGSCVQAEAAPAGLCGEQRGEDCAELHDYFNVLSYRSLGNCSFFTETG

SEQ ID NO: 677 - protein, 163 aa, Homo sapiens
MMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVALTRKKKALRIHSVEGDLRRKSA

SEQ ID NO: 678 - protein, 244 aa, Homo sapiens; identical to SEQ ID NO: 674

SEQ ID NO: 679 - protein, 263 aa, Homo sapiens
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEEKPQASPEGRPESETSCLVTTTDFQIQTEMAATMETSIFTTEYQVAVAGCVFLLISVLLLSGLTWQRRQRKSRRTI

SEQ ID NO: 680 - protein, 200 aa, Homo sapiens
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGEEKPQASPEGRPESETSCLVTTTDFQIQTEMAATMETSIFTTEYQVAVAGCVFLLISVLLLSGLTWQRRQRKSRRTI

SEQ ID NO: 681 - protein, 272 aa, Homo sapiens
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 682 - protein, 510 aa, Homo sapiens
MEDTKESNVKTFCSKNILAILGFSSIIAVIALLAVGLTQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHSTYVFLMVLFSLVLFTVAIIGLLIFHKPSYFWKDMV

SEQ ID NO: 683 - protein, 522 aa, Homo sapiens
MGREELFLTFSFSSGFQESNVKTFCSKNILAILGFSSIIAVIALLAVGLTQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHSTYVFLMVLFSLVLFTVAIIGLLIFHKPSYFWKDMV

SEQ ID NO: 684 - protein, 517 aa, Homo sapiens
MKGTKDLTSQQKESNVKTFCSKNILAILGFSSIIAVIALLAVGLTQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHSTYVFLMVLFSLVLFTVAIIGLLIFHKPSYFWKDMV

SEQ ID NO: 685 - protein, 63 aa, Homo sapiens
MERAREVIPRSQHQETPVYLGATAGMRLLRVPGSLLAKRKVPMAGLLSTICWANSVRKQGGSA

SEQ ID NO: 686 - protein, 402 aa, Homo sapiens
MERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHSTYVFLMVLFSLVLFTVAIIGLLIFHKPSYFWKDMV

SEQ ID NO: 687 - protein, 51 aa, Homo sapiens
MEDTKESNVKTFCSKNILAILGFSSIIAVIALLAVGLTQNKALPENVKDGK

SEQ ID NO: 688 - protein, 199 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 689 - protein, 168 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKM

SEQ ID NO: 690 - protein, 277 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 691 - protein, 255 aa, Homo sapiens
MAQHGAMGAFRALCGLALLCALSLGQRPTGGPGCGPGRLLLGTGTDARCCRVHTTRCCRDYPGEECCSEWDCMCVQPEFHCGDPCCTTCRHHPCPPGQGVQSQGKFSFGFQCIDCASGTFSGGHEGHCKPWTDCCWRCRRRPKTPEAASSPRKSGASDRQRRRGGWETCGCEPGRPPGPPTAASPSPGAPQAAGALRSALGRALLPWQQKWVQEGGSDQRPGPCSSAAAAGPCRRERETQSWPPSSLAGPDGVGS

SEQ ID NO: 692 - protein, 234 aa, Homo sapiens
MAQHGAMGAFRALCGLALLCALSLGQRPTGGPGCGPGRLLLGTGTDARCCRVHTTRCCRDYPGEECCSEWDCMCVQPEFHCGDPCCTTCRHHPCPPGQGVQSQGKFSFGFQCIDCASGTFSGGHEGHCKPWTDCTQFGFLTVFPGNKTHNAVCVPGSPPAEPLGWLTVVLLAVAACVLLLTSAQLGLHIWQLRKTQLLLEVPPSTEDARSCQFPEEERGERSAEEKGRLGDLWV

SEQ ID NO: 693 - protein, 241 aa, Homo sapiens
MAQHGAMGAFRALCGLALLCALSLGQRPTGGPGCGPGRLLLGTGTDARCCRVHTTRCCRDYPGEECCSEWDCMCVQPEFHCGDPCCTTCRHHPCPPGQGVQSQGKFSFGFQCIDCASGTFSGGHEGHCKPWTDCTQFGFLTVFPGNKTHNAVCVPGSPPAEPLGWLTVVLLAVAACVLLLTSAQLGLHIWQLRSQCMWPRETQLLLEVPPSTEDARSCQFPEEERGERSAEEKGRLGDLWV

SEQ ID NO: 694 - protein, 169 aa, Homo sapiens
MCVQPEFHCGDPCCTTCRHHPCPPGQGVQSQGKFSFGFQCIDCASGTFSGGHEGHCKPWTDCTQFGFLTVFPGNKTHNAVCVPGSPPAEPLGWLTVVLLAVAACVLLLTSAQLGLHIWQLRSQCMWPRETQLLLEVPPSTEDARSCQFPEEERGERSAEEKGRLGDLWV

SEQ ID NO: 695 - protein, 595 aa, Homo sapiens
MRVLLAALGLLFLGALRAFPQDRPFEDTCHGNPSHYYDKAVRRCCYRCPMGLFPTQQCPQRPTDCRKQCEPDYYLDEADRCTACVTCSRDDLVEKTPCAWNSSRVCECRPGMFCSTSAVNSCARCFFHSVCPAGMIVKFPGTAQKNTVCEPASPGVSPACASPENCKEPSSGTIPQAKPTPVSPATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSCARCVPYPICAAETVTKPQDMAEKDTTFEAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 696 - protein, 483 aa, Homo sapiens
MFCSTSAVNSCARCFFHSVCPAGMIVKFPGTAQKNTVCEPASPGVSPACASPENCKEPSSGTIPQAKPTPVSPATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSCARCVPYPICAAETVTKPQDMAEKDTTFEAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 697 - protein, 55 aa, Homo sapiens
MRVLLAALGLLFLGALRAFPQGCSRHSSAHRGLLTAGSSVSLTTTWMRPTAVQPA

SEQ ID NO: 698 - protein, 176 aa, Homo sapiens
MRIFAVFIFMTYWHLLNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 699 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 700 - protein, 551 aa, Homo sapiens
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 701 - protein, 146 aa, Homo sapiens
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETH

SEQ ID NO: 702 - protein, 88 aa, Homo sapiens
MAAPALSWRLPLLILLLPLATSWASAAVNGTSQFTCFYNSRANISCVWSQDGALQDTSCQVHAWPDRRRWNQTCELLPVSQASWACNL

SEQ ID NO: 703 - protein, 188 aa, Homo sapiens
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQ

SEQ ID NO: 704 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 705 - protein, 87 aa, Homo sapiens
ERDVVCGPSPADLSPGASSVTPPAPAREPGHSPQIISFFLALTSTALLFLLFFLTLRFSVVKRGRKKLLYIFKQHGSSGDTFTILES

SEQ ID NO: 706 - protein, 255 aa, Homo sapiens; identical to SEQ ID NO: 704

SEQ ID NO: 707 - protein, 92 aa, Homo sapiens
MHCTLTMETEAIDGYITCDNELSPEREHSNMAIDLTSSTPNGQHASPSHMTSSKSSFYDFFLCVCVSPATDFNLLGLWGPLTLWYSYCYRFC

SEQ ID NO: 708 - DNA, 660 bp, Homo sapiens
  source 1..660: mol_type = genomic DNA
atgcctcggggacgaaagagtcggcgccgccgtaatgcgagagccgcagaagagaaccgcaacaatcgcaaaatccaggcctcagaggcctccgagacccctatggccgcctctgtggtagcgagcacccccgaagacgacctgagcggccccgaggaagacccgagcactccagaggaggcctctaccacccctgaagaagcctcgagcactgcccaagcacaaaagccttcagtgccccggagcaattttcagggcaccaagaaaagtctcctgatgtctatattagcgctcatcttcatcatgggcaacagcgccaaggaagctctggtctggaaagtgctggggaagttaggaatgcagcctggacgtcagcacagcatctttggagatccgaagaagatcgtcacagaagagtttgtgcgcagagggtacctgatttataaaccggtgccccgtagcagtccggtggagtatgagttcttctgggggccccgagcacacgtggaatcgagcaaactgaaagtcatgcattttgtggcaagggttcgtaaccgatgctctaaagactggccttgtaattatgactgggattcggacgatgatgcagaggttgaggctatcctcaattcaggtgctaggggttattccgccccttaa

SEQ ID NO: 709 - protein, 219 aa, Homo sapiens; identical to SEQ ID NO: 615

SEQ ID NO: 710 - DNA, 64 bp, synthetic construct
  misc_feature 1..64: note = Adapter sequence
  source 1..64: mol_type = other DNA
agatcggaagagcacacgtctgaactccagtcacnnnnnnatctcgtatgccgtcttctgcttg

SEQ ID NO: 711 - DNA, 58 bp, synthetic construct
  misc_feature 1..58: note = Adapter sequence
  source 1..58: mol_type = other DNA
agatcggaagagcgtcgtgtagggaaagagtgtagatctcggtggtcgccgtatcatt

SEQ ID NO: 712 - protein, 116 aa, Homo sapiens
MLRLQMTDGHISCTAVEFSYMSKISLNTPPGTKVKLSGIVDIKNGFLLLNDSNTTVLGGEVEHLIEKWELQRSLSKHNRSNIGTEGGPPPFVPFGQFGRRNSQDKRKCEVLWCDPC

SEQ ID NO: 713 - protein, 651 aa, Homo sapiens
MLRLQMTDGHISCTAVEFSYMSKISLNTPPGTKVKLSGIVDIKNGFLLLNDSNTTVLGGEVEHLIEKWELQRSLSKHNRSNIGTEGGPPPFVPFGQKCVSHVQVDSRELDRRKTLQVTMPVKPTNDNDEFEKQRTAAIAEVAKSKETKTFGGGGGGARSNLNMNAAGNRNREVLQKEKSTKSEGKHEGVYRELVDEKALKHITEMGFSKEASRQALMDNGNNLEAALNVLLTSNKQKPVMGPPLRGRGKGRGRIRSEDEEDLGNARPSAPSTLFDFLESKMGTLNVEEPKSQPQQLHQGQYRSSNTEQNGVKDNNHLRHPPRNDTRQPRNEKPPRFQRDSQNSKSVLEGSGLPRNRGSERPSTSSVSEVWAEDRIKCDRPYSRYDRTKDTSYPLGSQHSDGAFKKRDNSMQSRSGKGPSFAEAKENPLPQGSVDYNNQKRGKRESQTSIPDYFYDRKSQTINNEAFSGIKIEKHFNVNTDYQNPVRSNSFIGVPNGEVEMPLKGRRIGPIKPAGPVTAVPCDDKIFYNSGPKRRSGPIKPEKILESSIPMEYAKMWKPGDECFALYWEDNKFYRAEVEALHSSGMTAVVKFIDYGNYEEVLLSNIKPIQTEAWEEEGTYDQTLEFRRGGDGQPRRSTRPTQQFYQPPRARN

SEQ ID NO: 714 - protein, 651 aa, Homo sapiens; identical to SEQ ID NO: 713

SEQ ID NO: 715 - protein, 129 aa, Homo sapiens
MLRLQMTDGHISCTAVEFSYMSKISLNTPPGTKVKLSGIVDIKNGFLLLNDSNTTVLGGEVEHLIEKWELQRSLSKHNRSNIGTEGGPPPFVPFGQKCVSHVQVDSRELDRRKTLQVTMPVKPTNDNDE

SEQ ID NO: 716 - protein, 744 aa, Homo sapiens
MAQVAGAALSQAGWYLSDEGIEACTSSPDKVNVNDIILIALNTDLRTIGKKFLPSDINSGKVEKLEGPCVLQIQKIRNVAAPKDNEESQAAPRMLRLQMTDGHISCTAVEFSYMSKISLNTPPGTKVKLSGIVDIKNGFLLLNDSNTTVLGGEVEHLIEKWELQRSLSKHNRSNIGTEGGPPPFVPFGQKCVSHVQVDSRELDRRKTLQVTMPVKPTNDNDEFEKQRTAAIAEVAKSKETKTFGGGGGGARSNLNMNAAGNRNREVLQKEKSTKSEGKHEGVYRELVDEKALKHITEMGFSKEASRQALMDNGNNLEAALNVLLTSNKQKPVMGPPLRGRGKGRGRIRSEDEEDLGNARPSAPSTLFDFLESKMGTLNVEEPKSQPQQLHQGQYRSSNTEQNGVKDNNHLRHPPRNDTRQPRNEKPPRFQRDSQNSKSVLEGSGLPRNRGSERPSTSSVSEVWAEDRIKCDRPYSRYDRTKDTSYPLGSQHSDGAFKKRDNSMQSRSGKGPSFAEAKENPLPQGSVDYNNQKRGKRESQTSIPDYFYDRKSQTINNEAFSGIKIEKHFNVNTDYQNPVRSNSFIGVPNGEVEMPLKGRRIGPIKPAGPVTAVPCDDKIFYNSGPKRRSGPIKPEKILESSIPMEYAKMWKPGDECFALYWEDNKFYRAEVEALHSSGMTAVVKFIDYGNYEEVLLSNIKPIQTEAWEEEGTYDQTLEFRRGGDGQPRRSTRPTQQFYQPPRARN

SEQ ID NO: 717 - protein, 651 aa, Homo sapiens; identical to SEQ ID NO: 713

SEQ ID NO: 718 - protein, 650 aa, Homo sapiens
MLRLQMTDGHISCTAVEFSYMSKISLNTPPGTKVKLSGIVDIKNGFLLLNDSNTTVLGGEVEHLIEKWELQRSLSKHNRSNIGTEGGPPPFVPFGQCVSHVQVDSRELDRRKTLQVTMPVKPTNDNDEFEKQRTAAIAEVAKSKETKTFGGGGGGARSNLNMNAAGNRNREVLQKEKSTKSEGKHEGVYRELVDEKALKHITEMGFSKEASRQALMDNGNNLEAALNVLLTSNKQKPVMGPPLRGRGKGRGRIRSEDEEDLGNARPSAPSTLFDFLESKMGTLNVEEPKSQPQQLHQGQYRSSNTEQNGVKDNNHLRHPPRNDTRQPRNEKPPRFQRDSQNSKSVLEGSGLPRNRGSERPSTSSVSEVWAEDRIKCDRPYSRYDRTKDTSYPLGSQHSDGAFKKRDNSMQSRSGKGPSFAEAKENPLPQGSVDYNNQKRGKRESQTSIPDYFYDRKSQTINNEAFSGIKIEKHFNVNTDYQNPVRSNSFIGVPNGEVEMPLKGRRIGPIKPAGPVTAVPCDDKIFYNSGPKRRSGPIKPEKILESSIPMEYAKMWKPGDECFALYWEDNKFYRAEVEALHSSGMTAVVKFIDYGNYEEVLLSNIKPIQTEAWEEEGTYDQTLEFRRGGDGQPRRSTRPTQQFYQPPRARN

SEQ ID NO: 719 - protein, 832 aa, Homo sapiens
MSEAGGAGPGGCGAGAGAGAGPGALPPQPAALPPAPPQGSPCAAAAGGSGACGPATAVAAAGTAEGPGGGGSARIAVKKAQLRSAPRAKKLEKLGVYSACKAEESCKCNGWKNPNPSPTPPRADLQQIIVSLTESCRSCSHALAAHVSHLENVSEEEMNRLLGIVLDVEYLFTCVHKEEDADTKQVYFYLFKLLRKSILQRGKPVVEGSLEKKPPFEKPSIEQGVNNFVQYKFSHLPAKERQTIVELAKMFLNRINYWHLEAPSQRRLRSPNDDISGYKENYTRWLCYCNVPQFCDSLPRYETTQVFGRTLLRSVFTVMRRQLLEQARQEKDKLPLEKRTLILTHFPKFLSMLEEEVYSQNSPIWDQDFLSASSRTSQLGIQTVINPPPVAGTISYNSTSSSLEQPNAGSSSPACKASSGLEANPGEKRKMTDSHVLEEAKKPRVMGDIPMELINEVMSTITDPAAMLGPETNFLSAHSARDEAARLEERRGVIEFHVVGNSLNQKPNKKILMWLVGLQNVFSHQLPRMPKEYITRLVFDPKHKTLALIKDGRVIGGICFRMFPSQGFTEIVFCAVTSNEQVKGYGTHLMNHLKEYHIKHDILNFLTYADEYAIGYFKKQGFSKEIKIPKTKYVGYIKDYEGATLMGCELNPRIPYTEFSVIIKKQKEIIKKLIERKQAQIRKVYPGLSCFKDGVRQIPIESIPGIRETGWKPSGKEKSKEPRDPDQLYSTLKSILQQVKSHQSAWPFMEPVKRTEAPGYYEVIRFPMDLKTMSERLKNRYYVSKKLFMADLQRVFTNCKEYNPPESEYYKCANILEKFFFSKIKEAGLIDK

SEQ ID NO: 720 - protein, 472 aa, Homo sapiens
MLGTVKMEGHETSDWNSYYADTQEAYSSVPVSNMNSGLGSMNSMNTYMTMNTMTTSGNMTPASFNMSYANPGLGAGLSPGAVAGMPGGSAGAMNSMTAAGVTAMGTALSPSGMGAMGAQQAASMNGLGPYAAAMNPCMSPMAYAPSNLGRSRAGGGGDAKTFKRSYPHAKPPYSYISLITMAIQQAPSKMLTLSEIYQWIMDLFPYYRQNQQRWQNSIRHSLSFNDCFVKVARSPDKPGKGSYWTLHPDSGNMFENGCYLRRQKRFKCEKQPGAGGGGGSGSGGSGAKGGPESRKDPSGASNPSADSPLHRGVHGKTGQLEGAPAPGPAASPQTLDHSGATATGGASELKTPASSTAPPISSGPGALASVPASHPAHGLAPHESQLHLKGDPHYSFNHPFSINNLMSSSEQQHKLDFKAYEQALQYSPYGSTLPASLPLGSASVTTRSPIEPSALEPAYYQGVYSRPVLNTS

SEQ ID NO: 721 - protein, 36 aa, Homo sapiens
MLGTVKMEGHETSDWNSYYADTQELAITLQKFNYHY

SEQ ID NO: 722 - protein, 531 aa, Homo sapiens
MVDVGKWPIFTLLSPQEIASIRKACVFGTSASEALYVTDNDEVFVFGLNYSNCLGTGDNQSTLVPKKLEGLCGKKIKSLSYGSGPHVLLSTEDGVVYAWGHNGYSQLGNGTTNQGIAPVQVCTNLLIKQVVEVACGSHHSMALAADGEVFAWGYNNCGQVGSGSTANQPTPRKVTNCLHIKRVVGIACGQTSSMAVLDNGEVYGWGYNGNGQLGLGNNGNQLTPVRVAALHSVCVNQIVCGYAHTLALTDEGLLYAWGANTYGQLGTGNKNNLLSPAHIMVEKERVVEIAACHSAHTSAAKTQGGHVYMWGQCRGQSVILPHLTHFSCTDDVFACFATPAVSWRLLSVEHEDFLTVAESLKKEFDSPETADLKFRIDGKYIHVHKAVLKIRCEHFRSMFQSYWNEDMKEVIEIDQFSYPVYRAFLQYLYTDTVDLPPEDAIGLLDLATSYCENRLKKLCQHIIKRGITVENAFSLFSAAVRYDAEDLEEFCFKFCINHLTEVTQTAAFWQMDGPLLKEFIAKASKCGAFKN

SEQ ID NO: 723 - protein, 531 aa, Homo sapiens; identical to SEQ ID NO: 722

SEQ ID NO: 724 - protein, 420 aa, Homo sapiens
MSGRPRTTSFAESCKPVQQPSAFGSMKVSRDKDGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKVFRPRTPPEAIALCSRLLEYTPTARLTPLEACAHSFFDELRDPNVKLPNGRDTPALFNFTTQELSSNPPLATILIPPHARIQAAASTPTNATAASDANTGDRGQTNNAASASASNST

SEQ ID NO: 725 - protein, 433 aa, Homo sapiens
MSGRPRTTSFAESCKPVQQPSAFGSMKVSRDKDGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKDSSGTGHFTSGVRVFRPRTPPEAIALCSRLLEYTPTARLTPLEACAHSFFDELRDPNVKLPNGRDTPALFNFTTQELSSNPPLATILIPPHARIQAAASTPTNATAASDANTGDRGQTNNAASASASNST

SEQ ID NO: 726 - protein, 326 aa, Homo sapiens
MEKDGLCRADQQYECVAEIGEGAYGKVFKARDLKNGGRFVALKRVRVQTGEEGMPLSTIREVAVLRHLETFEHPNVVRLFDVCTVSRTDRETKLTLVFEHVDQDLTTYLDKVPEPGVPTETIKDMMFQLLRGLDFLHSHRVVHRDLKPQNILVTSSGQIKLADFGLARIYSFQMALTSVVVTLWYRAPEVLLQSSYATPVDLWSVGCIFAEMFRRKPLFRGSSDVDQLGKILDVIGLPGEEDWPRDVALPRQAFHSKSAQPIEKFVTDIDELGKDLLLKCLTFNPAKRISAYSALSHPYFQDLERCKENLDSHLPPSQNTSELNTA

SEQ ID NO: 727 - protein, 326 aa, Homo sapiens; identical to SEQ ID NO: 726

SEQ ID NO: 728 - protein, 113 aa, Homo sapiens
MRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAK

SEQ ID NO: 729 - protein, 149 aa, Homo sapiens [sequence omitted]

SEQ ID NO: 730 - protein, 83 aa, Homo sapiens
MADQLTEEQIAEFKEAFSLFDKDGDGTITTKELGTVMRSLGQNPTEAELQDMINEVDEMIREADIDGDGQVNYEEFVQMMTAK

SEQ ID NO: 731 - protein, 113 aa, Homo sapiens; identical to SEQ ID NO: 728

SEQ ID NO: 732 - protein, 113 aa, Homo sapiens; identical to SEQ ID NO: 728

SEQ ID NO: 733 - protein, 113 aa, Homo sapiens; identical to SEQ ID NO: 728

SEQ ID NO: 734 - protein, 149 aa, Homo sapiens [sequence omitted]

SEQ ID NO: 735 - protein, 474 aa, Homo sapiens
MVQQTNNAENTEALLAGESSDSGAGLELGIASSPTPGSTASTGGKADDPSWCKTPSGHIKRPMNAFMVWSQIERRKIMEQSPDMHNAEISKRLGKRWKLLKDSDKIPFIREAERLRLKHMADYPDYKYRPRKKVKSGNANSSSSAAASSKPGEKGDKVGGSGGGGHGGGGGGGSSNAGGGGGGASGGGANSKPAQKKSCGSKVAGGAGGGVSKPHAKLILAGGGGGGKAAAAAAASFAAEQAGAAALLPLGAAADHHSLYKARTPSASASASSAASASAALAAPGKHLAEKKVKRVYLFGGLGTSSSPVGGVGAGADPSDPLGLYEEEGAGCSPDAPSLSGRSSAASSPAAGRSPADHRGYASLRAASPAPSSAPSHASSSASSHSSSSSSSGSSSSDDEFEDDLLDLNPSSNFESMSLGSFSSSSALDRDLDFNFEPGSGSHFEFPDYCTPEVSEMISGDWLESSISNLVFTY

SEQ ID NO: 736 - protein, 680 aa, Homo sapiens
MKMKKFQIPVSFQDLTVNFTQEEWQQLDPAQRLLYRDVMLENYSNLVSVGYHVSKPDVIFKLEQGEEPWIVEEFSNQNYPDIDDALEKNKEIQDKHLTQTVFFSNKTLITERENVFGKTLNLGMNSVPSRKMPYKCNPGGNSLKTNSEVIVAKKSKENRKIPDGYSGFGKHEKSHLGMKKYRYNPMRKASNQNENLILHQNIQILKQPFDYNKCGKTFFKRAILITQKGRQTERKPNECNECRKTFSKRSTLIVHQRIHTGEKPYVCSDCRKTFRVKTSLTRHRRIHTGERPYECSECRKTFIDKSALIVHQKIHGGEKSYECNECGKTFFRKSALAEHFRSHTGEKPYECKECGNAFSKKSYLVVHQRTHRGEKPNECKECGKTFFCQSALTAHQRIHTGEKPYECSECEKTFFCQSALNVHRRSHTGEKPYECSQCGKFLCTKSALIAHQITHRGKKSYECNECGKFFCHKSTLTIHQRTHTGEKHGVFNKCGRISIVKSNCSQCKRMNTKENLYECSEHGHAVSKNSHLIVHQRTIWERPYECNECGRTYCRKSALTHHQRTHTGQRPYECNECGKTFCQKFSFVEHQRTHTGEKPYECNECGKSFCHKSAFRVHRRIHTGEKPYECNQCGKTYRRLWTLTEHQKIHTGEKPYECNKCEKTFRHKSNFLLHQKSHKE

SEQ ID NO: 737 - protein, 642 aa, Homo sapiens
MLENYSNLVSVGYHVSKPDVIFKLEQGEEPWIVEEFSNQNYPDIDDALEKNKEIQDKHLTQTVFFSNKTLITERENVFGKTLNLGMNSVPSRKMPYKCNPGGNSLKTNSEVIVAKKSKENRKIPDGYSGFGKHEKSHLGMKKYRYNPMRKASNQNENLILHQNIQILKQPFDYNKCGKTFFKRAILITQKGRQTERKPNECNECRKTFSKRSTLIVHQRIHTGEKPYVCSDCRKTFRVKTSLTRHRRIHTGERPYECSECRKTFIDKSALIVHQKIHGGEKSYECNECGKTFFRKSALAEHFRSHTGEKPYECKECGNAFSKKSYLVVHQRTHRGEKPNECKECGKTFFCQSALTAHQRIHTGEKPYECSECEKTFFCQSALNVHRRSHTGEKPYECSQCGKFLCTKSALIAHQITHRGKKSYECNECGKFFCHKSTLTIHQRTHTGEKHGVFNKCGRISIVKSNCSQCKRMNTKENLYECSEHGHAVSKNSHLIVHQRTIWERPYECNECGRTYCRKSALTHHQRTHTGQRPYECNECGKTFCQKFSFVEHQRTHTGEKPYECNECGKSFCHKSAFRVHRRIHTGEKPYECNQCGKTYRRLWTLTEHQKIHTGEKPYECNKCEKTFRHKSNFLLHQKSHKE

SEQ ID NO: 738 - protein, 703 aa, Homo sapiens
MCVFLHLLSYIKDTSFIPGPDCELHPRGMAATGPCSEAPVQGCDAGELQQLGLCGVSDVQRSWSKFCNFPLTGYHVSKPDVIFKLEQGEEPWIVEEFSNQNYPDIDDALEKNKEIQDKHLTQTVFFSNKTLITERENVFGKTLNLGMNSVPSRKMPYKCNPGGNSLKTNSEVIVAKKSKENRKIPDGYSGFGKHEKSHLGMKKYRYNPMRKASNQNENLILHQNIQILKQPFDYNKCGKTFFKRAILITQKGRQTERKPNECNECRKTFSKRSTLIVHQRIHTGEKPYVCSDCRKTFRVKTSLTRHRRIHTGERPYECSECRKTFIDKSALIVHQKIHGGEKSYECNECGKTFFRKSALAEHFRSHTGEKPYECKECGNAFSKKSYLVVHQRTHRGEKPNECKECGKTFFCQSALTAHQRIHTGEKPYECSECEKTFFCQSALNVHRRSHTGEKPYECSQCGKFLCTKSALIAHQITHRGKKSYECNECGKFFCHKSTLTIHQRTHTGEKHGVFNKCGRISIVKSNCSQCKRMNTKENLYECSEHGHAVSKNSHLIVHQRTIWERPYECNECGRTYCRKSALTHHQRTHTGQRPYECNECGKTFCQKFSFVEHQRTHTGEKPYECNECGKSFCHKSAFRVHRRIHTGEKPYECNQCGKTYRRLWTLTEHQKIHTGEKPYECNKCEKTFRHKSNFLLHQKSHKE

SEQ ID NO: 739 - protein, 679 aa, Homo sapiens
MENEKEIPVSFQDLTVNFTQEEWQQLDPAQRLLYRDVMLENYSNLVSVGYHVSKPDVIFKLEQGEEPWIVEEFSNQNYPDIDDALEKNKEIQDKHLTQTVFFSNKTLITERENVFGKTLNLGMNSVPSRKMPYKCNPGGNSLKTNSEVIVAKKSKENRKIPDGYSGFGKHEKSHLGMKKYRYNPMRKASNQNENLILHQNIQILKQPFDYNKCGKTFFKRAILITQKGRQTERKPNECNECRKTFSKRSTLIVHQRIHTGEKPYVCSDCRKTFRVKTSLTRHRRIHTGERPYECSECRKTFIDKSALIVHQKIHGGEKSYECNECGKTFFRKSALAEHFRSHTGEKPYECKECGNAFSKKSYLVVHQRTHRGEKPNECKECGKTFFCQSALTAHQRIHTGEKPYECSECEKTFFCQSALNVHRRSHTGEKPYECSQCGKFLCTKSALIAHQITHRGKKSYECNECGKFFCHKSTLTIHQRTHTGEKHGVFNKCGRISIVKSNCSQCKRMNTKENLYECSEHGHAVSKNSHLIVHQRTIWERPYECNECGRTYCRKSALTHHQRTHTGQRPYECNECGKTFCQKFSFVEHQRTHTGEKPYECNECGKSFCHKSAFRVHRRIHTGEKPYECNQCGKTYRRLWTLTEHQKIHTGEKPYECNKCEKTFRHKSNFLLHQKSHKE

SEQ ID NO: 740 - protein, 173 aa, Homo sapiens
MLENYSNLVSVGYHVSKPDVIFKLEQGEEPWIVEEFSNQNYPDIDDALEKNKEIQDKHLTQTVFFSNKTLITERENVFGKTLNLGMNSVPSRKMPYKCNPGGNSLKTNSEVIVAKKSKENRKIPDGYSGFGKHEKSHLGMKKYRYNPMRKASNQNENLILHQNIQILKQPFDY

SEQ ID NO: 741 - protein, 679 aa, Homo sapiens; identical to SEQ ID NO: 739

SEQ ID NO: 742 - protein, 1455 aa, Homo sapiens
MGGACSSFTTSSSPTIYSTSVTDSKAMQVESCSSAVGVSNRGVSEKQLTSNTVQQHPSTPKRHTVLYISPPPEDLLDNSRMSCQDEGCGLESEQSCSMWMEDSPSNFSNMSTSSYNDNTEVPRKSRKRNPKQRPGVKRRDCEESNMDIFDADSAKAPHYVLSQLTTDNKGNSKAGNGTLENQKGTGVKKSPMLCGQYPVKSEGKELKIVVQPETQHRARYLTEGSRGSVKDRTQQGFPTVKLEGHNEPVVLQVFVGNDSGRVKPHGFYQACRVTGRNTTPCKEVDIEGTTVIEVGLDPSNNMTLAVDCVGILKLRNADVEARIGIAGSKKKSTRARLVFRVNIMRKDGSTLTLQTPSSPILCTQPAGVPEILKKSLHSCSVKGEEEVFLIGKNFLKGTKVIFQENVSDENSWKSEAEIDMELFHQNHLIVKVPPYHDQHITLPVSVGIYVVTNAGRSHDVQPFTYTPDPAAAGALNVNVKKEISSPARPCSFEEAMKAMKTTGCNLDKVNIIPNALMTPLIPSSMIKSEDVTPMEVTAEKRSSTIFKTTKSVGSTQQTLENISNIAGNGSFSSPSSSHLPSENEKQQQIQPKAYNPETLTTIQTQDISQPGTFPAVSASSQLPNSDALLQQATQFQTRETQSREILQSDGTVVNLSQLTEASQQQQQSPLQEQAQTLQQQISSNIFPSPNSVSQLQNTIQQLQAGSFTGSTASGSSGSVDLVQQVLEAQQQLSSVLFSAPDGNENVQEQLSADIFQQVSQIQSGVSPGMFSSTEPTVHTRPDNLLPGRAESVHPQSENTLSNQQQQQQQQQQVMESSAAMVMEMQQSICQAAAQIQSELFPSTASANGNLQQSPVYQQTSHMMSALSTNEDMQMQCELFSSPPAVSGNETSTTTTQQVATPGTTMFQTSSSGDGEETGTQAKQIQNSVFQTMVQMQHSGDNQPQVNLFSSTKSMMSVQNSGTQQQGNGLFQQGNEMMSLQSGNFLQQSSHSQAQLFHPQNPIADAQNLSQETQGSLFHSPNPIVHSQTSTTSSEQMQPPMFHSQSTIAVLQGSSVPQDQQSTNIFLSQSPMNNLQTNTVAQEAFFAAPNSISPLQSTSNSEQQAAFQQQAPISHIQTPMLSQEQAQPPQQGLFQPQVALGSLPPNPMPQSQQGTMFQSQHSIVAMQSNSPSQEQQQQQQQQQQQQQQQQQSILFSNQNTMATMASPKQPPPNMIFNPNQNPMANQEQQNQSIFHQQSNMAPMNQEQQPMQFQSQSTVSSLQNPGPTQSESSQTPLFHSSPQIQLVQGSPSSQEQQVTLFLSPASMSALQTSINQQDMQQSPLYSPQNNMPGIQGATSSPQPQATLFHNTAGGTMNQLQNSPGSSQQTSGMFLFGIQNNCSQLLTSGPATLPDQLMAISQPGQPQNEGQPPVTTLLSQQMPENSPLASSINTNQNIEKIDLLVSLQNQGNNLTGSF

SEQ ID NO: 743 - protein, 70 aa, Homo sapiens
XIIPTLPSDKLSKIQTLKLAARYIDFLYQVLQSDELDSKMASCSYVAHERLSYAFSVWRMEGAWSMSASH

SEQ ID NO: 744 - protein, 135 aa, Homo sapiens
XPAQGKRGKKSAGCGGGGGAGGGGGSSSGGGSPQSYEELQTQRVMANVRERQRTQSLNEAFAALRKIIPTLPSDKLSKIQTLKLAARYIDFLYQVLQSDELDSKMASCSYVAHERLSYAFSVWRMEGAWSMSASH

SEQ ID NO: 745 - protein, 202 aa, Homo sapiens
MMQDVSSSPVSPADDSLSNSEEEPDRQQPPSGKRGGRKRRSSRRSAGGGAGPGGAAGGGVGGGDEPGSPAQGKRGKKSAGCGGGGGAGGGGGSSSGGGSPQSYEELQTQRVMANVRERQRTQSLNEAFAALRKIIPTLPSDKLSKIQTLKLAARYIDFLYQVLQSDELDSKMASCSYVAHERLSYAFSVWRMEGAWSMSASH

SEQ ID NO: 746 - protein, 636 aa, Homo sapiens
MAQSTATSPDGGTTFEHLWSSLEPDSTYFDLPQSSRGNNEVVGGTDSSMDVFHLEGMTTSVMAQFNLLSSTMDQMSSRAASASPYTPEHAASVPTHSPYAQPSSTFDTMSPAPVIPSNTDYPGPHHFEVTFQQSSTAKSATWTYSPLLKKLYCQIAKTCPIQIKVSTPPPPGTAIRAMPVYKKAEHVTDVVKRCPNHELGRDFNEGQSAPASHLIRVEGNNLSQYVDDPVTGRQSVVVPYEPPQVGTEFTTILYNFMCNSSCVGGMNRRPILIIITLEMRDGQVLGRRSFEGRICACPGRDRKADEDHYREQQALNESSAKNGAASKRAFKQSPPAVPALGAGVKKRRHGDEDTYYLQVRGRENFEILMKLKESLELMELVPQPLVDSYRQQQQLLQRPSHLQPPSYGPVLSPMNKVHGGMNKLPSVNQLVGQPPPHSSAATPNLGPVGPGMLNNHGHAVPANGEMSSSHSAQSMVSGSHCTPPPPYHADPSLVSFLTGLGCPNCIEYFTSQGLQSIYHLQNLTIEDLGALKIPEQYRMTIWRGLQDLKQGHDYSTAQQLLRSSNAATISIGGSGELQRQRVMEAVHFRVRHTITIPNRGGPGGGPDEWADFGFDLPDCKARKQPIKEEFTEAEIH

SEQ ID NO: 747 - protein, 403 aa, Homo sapiens
MAQSTATSPDGGTTFEHLWSSLEPDSTYFDLPQSSRGNNEVVGGTDSSMDVFHLEGMTTSVMAQFNLLSSTMDQMSSRAASASPYTPEHAASVPTHSPYAQPSSTFDTMSPAPVIPSNTDYPGPHHFEVTFQQSSTAKSATWTYSPLLKKLYCQIAKTCPIQIKVSTPPPPGTAIRAMPVYKKAEHVTDVVKRCPNHELGRDFNEGQSAPASHLIRVEGNNLSQYVDDPVTGRQSVVVPYEPPQVGTEFTTILYNFMCNSSCVGGMNRRPILIIITLEMRDGQVLGRRSFEGRICACPGRDRKADEDHYREQQALNESSAKNGAASKRAFKQSPPAVPALGAGVKKRRHGDEDTYYLQVRGRENFEILMKLKESLELMELVPQPLVDSYRQQQQLLQRPTWGP

SEQ ID NO: 748 - protein, 499 aa, Homo sapiens
MAQSTATSPDGGTTFEHLWSSLEPDSTYFDLPQSSRGNNEVVGGTDSSMDVFHLEGMTTSVMAQFNLLSSTMDQMSSRAASASPYTPEHAASVPTHSPYAQPSSTFDTMSPAPVIPSNTDYPGPHHFEVTFQQSSTAKSATWTYSPLLKKLYCQIAKTCPIQIKVSTPPPPGTAIRAMPVYKKAEHVTDVVKRCPNHELGRDFNEGQSAPASHLIRVEGNNLSQYVDDPVTGRQSVVVPYEPPQVGTEFTTILYNFMCNSSCVGGMNRRPILIIITLEMRDGQVLGRRSFEGRICACPGRDRKADEDHYREQQALNESSAKNGAASKRAFKQSPPAVPALGAGVKKRRHGDEDTYYLQVRGRENFEILMKLKESLELMELVPQPLVDSYRQQQQLLQRPSHLQPPSYGPVLSPMNKVHGGMNKLPSVNQLVGQPPPHSSAATPNLGPVGPGMLNNHGHAVPANGEMSSSHSAQSMVSGSHCTPPPPYHADPSLVRTWGP

SEQ ID NO: 749 - protein, 555 aa, Homo sapiens
MAQSTATSPDGGTTFEHLWSSLEPDSTYFDLPQSSRGNNEVVGGTDSSMDVFHLEGMTTSVMAQFNLLSSTMDQMSSRAASASPYTPEHAASVPTHSPYAQPSSTFDTMSPAPVIPSNTDYPGPHHFEVTFQQSSTAKSATWTYSPLLKKLYCQIAKTCPIQIKVSTPPPPGTAIRAMPVYKKAEHVTDVVKRCPNHELGRDFNEGQSAPASHLIRVEGNNLSQYVDDPVTGRQSVVVPYEPPQVGTEFTTILYNFMCNSSCVGGMNRRPILIIITLEMRDGQVLGRRSFEGRICACPGRDRKADEDHYREQQALNESSAKNGAASKRAFKQSPPAVPALGAGVKKRRHGDEDTYYLQVRGRENFEILMKLKESLELMELVPQPLVDSYRQQQQLLQRPPRDAQQPWPRSASQRRDEQQPQRPVHGLGVPLHSATPLPRRPQPRQDLGALKIPEQYRMTIWRGLQDLKQGHDYSTAQQLLRSSNAATISIGGSGELQRQRVMEAVHFRVRHTITIPNRGGPGGGPDEWADFGFDLPDCKARKQPIKEEFTEAEIH

SEQ ID NO: 750 - protein, 540 aa, Homo sapiens
MAQSTATSPDGGTTFEHLWSSLEPDSTYFDLPQSSRGNNEVVGGTDSSMDVFHLEGMTTSVMAQFNLLSSTMDQMSSRAASASPYTPEHAASVPTHSPYAQPSSTFDTMSPAPVIPSNTDYPGPHHFEVTFQQSSTAKSATWTYSPLLKKLYCQIAKTCPIQIKVSTPPPPGTAIRAMPVYKKAEHVTDVVKRCPNHELGRDFNEGQSAPASHLIRVEGNNLSQYVDDPVTGRQSVVVPYEPPQVGTEFTTILYNFMCNSSCVGGMNRRPILIIITLEMRDGQVLGRRSFEGRICACPGRDRKADEDHYREQQALNESSAKNGAASKRAFKQSPPAVPALGAGVKKRRHGDEDTYYLQVRGRENFEILMKLKESLELMELVPQPLVDSYRQQQQLLQRPFLTGLGCPNCIEYFTSQGLQSIYHLQNLTIEDLGALKIPEQYRMTIWRGLQDLKQGHDYSTAQQLLRSSNAATISIGGSGELQRQRVMEAVHFRVRHTITIPNRGGPGGGPDEWADFGFDLPDCKARKQPIKEEFTEAEIH

SEQ ID NO: 751 - protein, 450 aa, Homo sapiens
MLYVGDPARHLATAQFNLLSSTMDQMSSRAASASPYTPEHAASVPTHSPYAQPSSTFDTMSPAPVIPSNTDYPGPHHFEVTFQQSSTAKSATWTYSPLLKKLYCQIAKTCPIQIKVSTPPPPGTAIRAMPVYKKAEHVTDVVKRCPNHELGRDFNEGQSAPASHLIRVEGNNLSQYVDDPVTGRQSVVVPYEPPQVGTEFTTILYNFMCNSSCVGGMNRRPILIIITLEMRDGQVLGRRSFEGRICACPGRDRKADEDHYREQQALNESSAKNGAASKRAFKQSPPAVPALGAGVKKRRHGDEDTYYLQVRGRENFEILMKLKESLELMELVPQPLVDSYRQQQQLLQRPSHLQPPSYGPVLSPMNKVHGGMNKLPSVNQLVGQPPPHSSAATPNLGPVGPGMLNNHGHAVPANGEMSSSHSAQSMVSGSHCTPPPPYHADPSLVRTWGP

SEQ ID NO: 752 - protein, 426 aa, Homo sapiens
MLYVGDPARHLATAQFNLLSSTMDQMSSRAASASPYTPEHAASVPTHSPYAQPSSTFDTMSPAPVIPSNTDYPGPHHFEVTFQQSSTAKSATWTYSPLLKKLYCQIAKTCPIQIKVSTPPPPGTAIRAMPVYKKAEHVTDVVKRCPNHELGRDFNEGQSAPASHLIRVEGNNLSQYVDDPVTGRQSVVVPYEPPQVGTEFTTILYNFMCNSSCVGGMNRRPILIIITLEMRDGQVLGRRSFEGRICACPGRDRKADEDHYREQQALNESSAKNGAASKRAFKQSPPAVPALGAGVKKRRHGDEDTYYLQVRGRENFEILMKLKESLELMELVPQPLVDSYRQQQQLLQRPPRDAQQPWPRSASQRRDEQQPQRPVHGLGVPLHSATPLPRRPQPRQFFNRIGVSKLHRVFHLPRVTEHLPPAEPDH

SEQ ID NO: 753 - protein, 587 aa, Homo sapiens
MLYVGDPARHLATAQFNLLSSTMDQMSSRAASASPYTPEHAASVPTHSPYAQPSSTFDTMSPAPVIPSNTDYPGPHHFEVTFQQSSTAKSATWTYSPLLKKLYCQIAKTCPIQIKVSTPPPPGTAIRAMPVYKKAEHVTDVVKRCPNHELGRDFNEGQSAPASHLIRVEGNNLSQYVDDPVTGRQSVVVPYEPPQVGTEFTTILYNFMCNSSCVGGMNRRPILIIITLEMRDGQVLGRRSFEGRICACPGRDRKADEDHYREQQALNESSAKNGAASKRAFKQSPPAVPALGAGVKKRRHGDEDTYYLQVRGRENFEILMKLKESLELMELVPQPLVDSYRQQQQLLQRPSHLQPPSYGPVLSPMNKVHGGMNKLPSVNQLVGQPPPHSSAATPNLGPVGPGMLNNHGHAVPANGEMSSSHSAQSMVSGSHCTPPPPYHADPSLVSFLTGLGCPNCIEYFTSQGLQSIYHLQNLTIEDLGALKIPEQYRMTIWRGLQDLKQGHDYSTAQQLLRSSNAATISIGGSGELQRQRVMEAVHFRVRHTITIPNRGGPGGGPDEWADFGFDLPDCKARKQPIKEEFTEAEIH

SEQ ID NO: 754 - protein, 565 aa, Homo sapiens
MDQMSSRAASASPYTPEHAASVPTHSPYAQPSSTFDTMSPAPVIPSNTDYPGPHHFEVTFQQSSTAKSATWTYSPLLKKLYCQIAKTCPIQIKVSTPPPPGTAIRAMPVYKKAEHVTDVVKRCPNHELGRDFNEGQSAPASHLIRVEGNNLSQYVDDPVTGRQSVVVPYEPPQVGTEFTTILYNFMCNSSCVGGMNRRPILIIITLEMRDGQVLGRRSFEGRICACPGRDRKADEDHYREQQALNESSAKNGAASKRAFKQSPPAVPALGAGVKKRRHGDEDTYYLQVRGRENFEILMKLKESLELMELVPQPLVDSYRQQQQLLQRPSHLQPPSYGPVLSPMNKVHGGMNKLPSVNQLVGQPPPHSSAATPNLGPVGPGMLNNHGHAVPANGEMSSSHSAQSMVSGSHCTPPPPYHADPSLVSFLTGLGCPNCIEYFTSQGLQSIYHLQNLTIEDLGALKIPEQYRMTIWRGLQDLKQGHDYSTAQQLLRSSNAATISIGGSGELQRQRVMEAVHFRVRHTITIPNRGGPGGGPDEWADFGFDLPDCKARKQPIKEEFTEAEIH

SEQ ID NO: 755 - protein, 555 aa, Homo sapiens; identical to SEQ ID NO: 749

SEQ ID NO: 756 - protein, 540 aa, Homo sapiens; identical to SEQ ID NO: 750

SEQ ID NO: 757 - protein, 602 aa, Homo sapiens
MAAEEEAAAGGKVLREENQCIAPVVSSRVSPGTRPTAMGSFSSHMTEFPRKRKGSDSDPSQVEDGEHQVKMKAFREAHSQTEKRRRDKMNNLIEELSAMIPQCNPMARKLDKLTVLRMAVQHLRSLKGLTNSYVGSNYRPSFLQDNELRHLILKTAEGFLFVVGCERGKILFVSKSVSKILNYDQASLTGQSLFDFLHPKDVAKVKEQLSSFDISPREKLIDAKTGLQVHSNLHAGRTRVYSGSRRSFFCRIKSCKISVKEEHGCLPNSKKKEHRKFYTIHCTGYLRSWPPNIVGMEEERNSKKDNSNFTCLVAIGRLQPYIVPQNSGEINVKPTEFITRFAVNGKFVYVDQRATAILGYLPQELLGTSCYEYFHQDDHNNLTDKHKAVLQSKEKILTDSYKFRAKDGSFVTLKSQWFSFTNPWTKELEYIVSVNTLVLGHSEPGEASFLPCSSQSSEESSRQSCMSVPGMSTGTVLGAGSIGTDIANEILDLQRLQSSSYLDDSSPTGLMKDTHTVNCRSMSNKELFPPSPSEMGELEATRQNQSTVAVHSHEPLLSDGAQLDFDALCDNDDTAMAAFMNYLEAEGGLGDPGDFSDIQWTL

SEQ ID NO: 758 - protein, 599 aa, Homo sapiens
MAAEEEAAAGGEVAGGEATAPGKVLREENQCIAPVVSSRVSPGTRPTAMGSFSSHMTEFPRKRKGSDSDPSQEAHSQTEKRRRDKMNNLIEELSAMIPQCNPMARKLDKLTVLRMAVQHLRSLKGLTNSYVGSNYRPSFLQDNELRHLILKTAEGFLFVVGCERGKILFVSKSVSKILNYDQASLTGQSLFDFLHPKDVAKVKEQLSSFDISPREKLIDAKTGLQVHSNLHAGRTRVYSGSRRSFFCRIKSCKISVKEEHGCLPNSKKKEHRKFYTIHCTGYLRSWPPNIVGMEEERNSKKDNSNFTCLVAIGRLQPYIVPQNSGEINVKPTEFITRFAVNGKFVYVDQRATAILGYLPQELLGTSCYEYFHQDDHNNLTDKHKAVLQSKEKILTDSYKFRAKDGSFVTLKSQWFSFTNPWTKELEYIVSVNTLVLGHSEPGEASFLPCSSQSSEESSRQSCMSVPGMSTGTVLGAGSIGTDIANEILDLQRLQSSSYLDDSSPTGLMKDTHTVNCRSMSNKELFPPSPSEMGELEATRQNQSTVAVHSHEPLLSDGAQLDFDALCDNDDTAMAAFMNYLEAEGGLGDPGDFSDIQWTL

SEQ ID NO: 759 - protein, 540 aa, Homo sapiens
MAAEEEAAAGGEVAGGEATAPGKVLREENQCIAPVVSSRVSPGTRPTAMGSFSSHMTEFPRKRKGSDSDPSQEAHSQTEKRRRDKMNNLIEELSAMIPQCNPMARKLDKLTVLRMAVQHLRSLKGLTNSYVGSNYRPSFLQDNELRHLILKTAEGFLFVVGCERGKILFVSKSVSKILNYDQASLTGQSLFDFLHPKDVAKVKEQLSSFDISPREKLIDAKTGLQVHSNLHAGRTRVYSGSRRSFFCRIKSCKISVKEEHGCLPNSKKKEHRKFYTIHCTGYLRSWPPNIVGMEEERNSKKDNSNFTCLVAIGRLQPYIVPQNSGEINVKPTEFITRFAVNGKFVYVDQRATAILGYLPQELLGTSCYEYFHQDDHNNLTDKHKAVLQSKEKILTDSYKFRAKDGSFVTLKSQWFSFTNPWTKELEYIVSVNTLVLGHSEPGEASFLPCSSQSSEESSRQSCMSVPGMSTGTVLGAGSIGTDIANEILDLQRLQSSSYLDDSSPTGLMKDTHTVNCRSVMVHSWISMPYVTMMTQPWLHL

SEQ ID NO: 760 - protein, 622 aa, Homo sapiens
MAAEEEAAAGGKVLREENQCIAPVVSSRVSPGTRPTAMGSFSSHMTEFPRKRKGSDSDPSQSGIMTEKVVEKLSQNPLTYLLSTRIEISASSGSREAHSQTEKRRRDKMNNLIEELSAMIPQCNPMARKLDKLTVLRMAVQHLRSLKGLTNSYVGSNYRPSFLQDNELRHLILKTAEGFLFVVGCERGKILFVSKSVSKILNYDQASLTGQSLFDFLHPKDVAKVKEQLSSFDISPREKLIDAKTGLQVHSNLHAGRTRVYSGSRRSFFCRIKSCKISVKEEHGCLPNSKKKEHRKFYTIHCTGYLRSWPPNIVGMEEERNSKKDNSNFTCLVAIGRLQPYIVPQNSGEINVKPTEFITRFAVNGKFVYVDQRATAILGYLPQELLGTSCYEYFHQDDHNNLTDKHKAVLQSKEKILTDSYKFRAKDGSFVTLKSQWFSFTNPWTKELEYIVSVNTLVLGHSEPGEASFLPCSSQSSEESSRQSCMSVPGMSTGTVLGAGSIGTDIANEILDLQRLQSSSYLDDSSPTGLMKDTHTVNCRSMSNKELFPPSPSEMGELEATRQNQSTVAVHSHEPLLSDGAQLDFDALCDNDDTAMAAFMNYLEAEGGLGDPGDFSDIQWTL

SEQ ID NO: 761 - protein, 588 aa, Homo sapiens
MAAEEEAAAGGKVLREENQCIAPVVSSRVSPGTRPTAMGSFSSHMTEFPRKRKGSDSDPSQEAHSQTEKRRRDKMNNLIEELSAMIPQCNPMARKLDKLTVLRMAVQHLRSLKGLTNSYVGSNYRPSFLQDNELRHLILKTAEGFLFVVGCERGKILFVSKSVSKILNYDQASLTGQSLFDFLHPKDVAKVKEQLSSFDISPREKLIDAKTGLQVHSNLHAGRTRVYSGSRRSFFCRIKSCKISVKEEHGCLPNSKKKEHRKFYTIHCTGYLRSWPPNIVGMEEERNSKKDNSNFTCLVAIGRLQPYIVPQNSGEINVKPTEFITRFAVNGKFVYVDQRATAILGYLPQELLGTSCYEYFHQDDHNNLTDKHKAVLQSKEKILTDSYKFRAKDGSFVTLKSQWFSFTNPWTKELEYIVSVNTLVLGHSEPGEASFLPCSSQSSEESSRQSCMSVPGMSTGTVLGAGSIGTDIANEILDLQRLQSSSYLDDSSPTGLMKDTHTVNCRSMSNKELFPPSPSEMGELEATRQNQSTVAVHSHEPLLSDGAQLDFDALCDNDDTAMAAFMNYLEAEGGLGDPGDFSDIQWTL

SEQ ID NO: 762 - protein, 636 aa, Homo sapiens
MAAEEEAAAGGKVLREENQCIAPVVSSRVSPGTRPTAMGSFSSHMTEFPRKRKGSDSDPSQSGIMTEKVVEKLSQNPLTYLLSTRIEISASSGSRVEDGEHQVKMKAFREAHSQTEKRRRDKMNNLIEELSAMIPQCNPMARKLDKLTVLRMAVQHLRSLKGLTNSYVGSNYRPSFLQDNELRHLILKTAEGFLFVVGCERGKILFVSKSVSKILNYDQASLTGQSLFDFLHPKDVAKVKEQLSSFDISPREKLIDAKTGLQVHSNLHAGRTRVYSGSRRSFFCRIKSCKISVKEEHGCLPNSKKKEHRKFYTIHCTGYLRSWPPNIVGMEEERNSKKDNSNFTCLVAIGRLQPYIVPQNSGEINVKPTEFITRFAVNGKFVYVDQRATAILGYLPQELLGTSCYEYFHQDDHNNLTDKHKAVLQSKEKILTDSYKFRAKDGSFVTLKSQWFSFTNPWTKELEYIVSVNTLVLGHSEPGEASFLPCSSQSSEESSRQSCMSVPGMSTGTVLGAGSIGTDIANEILDLQRLQSSSYLDDSSPTGLMKDTHTVNCRSMSNKELFPPSPSEMGELEATRQNQSTVAVHSHEPLLSDGAQLDFDALCDNDDTAMAAFMNYLEAEGGLGDPGDFSDIQWTL

SEQ ID NO: 763 - protein, 588 aa, Homo sapiens
XPVVSSRVSPGTRPTAMGSFSSHMTEFPRKRKGSDSDPSHQNPLTYLLSTRIEISASSGSREAHSQTEKRRRDKMNNLIEELSAMIPQCNPMARKLDKLTVLRMAVQHLRSLKGLTNSYVGSNYRPSFLQDNELRHLILKTAEGFLFVVGCERGKILFVSKSVSKILNYDQASLTGQSLFDFLHPKDVAKVKEQLSSFDISPREKLIDAKTGLQVHSNLHAGRTRVYSGSRRSFFCRIKSCKISVKEEHGCLPNSKKKEHRKFYTIHCTGYLRSWPPNIVGMEEERNSKKDNSNFTCLVAIGRLQPYIVPQNSGEINVKPTEFITRFAVNGKFVYVDQRATAILGYLPQELLGTSCYEYFHQDDHNNLTDKHKAVLQSKEKILTDSYKFRAKDGSFVTLKSQWFSFTNPWTKELEYIVSVNTLVLGHSEPGEASFLPCSSQSSEESSRQSCMSVPGMSTGTVLGAGSIGTDIANEILDLQRLQSSSYLDDSSPTGLMKDTHTVNCRSMSNKELFPPSPSEMGELEATRQNQSTVAVHSHEPLLSDGAQLDFDALCDNDDTAMAAFMNYLEAEGGLGDPGDFSDIQWTL

SEQ ID NO: 764 - protein, 551 aa, Homo sapiens
MGSFSSHMTEFPRKRKGSDSDPSQEAHSQTEKRRRDKMNNLIEELSAMIPQCNPMARKLDKLTVLRMAVQHLRSLKGLTNSYVGSNYRPSFLQDNELRHLILKTAEGFLFVVGCERGKILFVSKSVSKILNYDQASLTGQSLFDFLHPKDVAKVKEQLSSFDISPREKLIDAKTGLQVHSNLHAGRTRVYSGSRRSFFCRIKSCKISVKEEHGCLPNSKKKEHRKFYTIHCTGYLRSWPPNIVGMEEERNSKKDNSNFTCLVAIGRLQPYIVPQNSGEINVKPTEFITRFAVNGKFVYVDQRATAILGYLPQELLGTSCYEYFHQDDHNNLTDKHKAVLQSKEKILTDSYKFRAKDGSFVTLKSQWFSFTNPWTKELEYIVSVNTLVLGHSEPGEASFLPCSSQSSEESSRQSCMSVPGMSTGTVLGAGSIGTDIANEILDLQRLQSSSYLDDSSPTGLMKDTHTVNCRSMSNKELFPPSPSEMGELEATRQNQSTVAVHSHEPLLSDGAQLDFDALCDNDDTAMAAFMNYLEAEGGLGDPGDFSDIQWTL

SEQ ID NO: 765 - protein, 125 aa, Homo sapiens
MPHSSDSSDSSFSRSPPPGKQDSSDDVRRVQRREKNRIAAQKSRQRQTQKADTLHLESEDLEKQNAALRKEIKQLTEELKYFTSVLNSHEPLCSVLAASTPSPPEVVYSAHAFHQPHVSSPRFQP

SEQ ID NO: 766 - protein, 73 aa, Homo sapiens
MPHSSDSSDSSFSRSPPPGKQDSSDDVRRVQRREKNRIAAQKSRQRQTQKSIKKDAQVPWHRARRAGNGYFSK

SEQ ID NO: 767 - protein, 202 aa, Homo sapiens
MATLIYVDKENGEPGTRVVAKDGLKLGSGPSIKALDGRSQVSTPRFGKTFDAPPALPKATRKALGTVNRATEKSVKTKGPLKQKQPSFSAKKMTEKTVKAKSSVPASDDAYPEIEKFFPFNPLDFESFDLPEEHQIAHLPLSGVPLMILDEERELEKLFQLGPPSPVKMPSPPWESNLLQSPSSILSTLDVELPPVCCDIDI

SEQ ID NO: 768 - protein, 121 aa, Homo sapiens
MPLPKVGGGFGGERSSLYTQYLSCTGILLLEYSYGKTFSSAIKALDGRSQVSTPRFGKTFDAPPALPKATRKALGTVNRATEKSVKTKGPLKQKQPSFSAKKMTEKTVKAKSSVPASDDAY

SEQ ID NO: 769 - protein, 202 aa, Homo sapiens; identical to SEQ ID NO: 767

SEQ ID NO: 770 - protein, 202 aa, Homo sapiens; identical to SEQ ID NO: 767

SEQ ID NO: 771 - protein, 2406 aa, Homo sapiens
MDPEQSVKGTKKAEGSPRKRLTKGEAIQTSVSSSVPYPGSGTAATQESPAQELLAPQPFPGPSSVLREGSQEKTGQQQKPPKRPPIEASVHISQLPQHPLTPAFMSPGKPEHLLEGSTWQLVDPMRPGPSGSFVAPGLHPQSQLLPSHASIIPPEDLPGVPKVFVPRPSQVSLKPTEEAHKKERKPQKPGKYICQYCSRPCAKPSVLQKHIRSHTGERPYPCGPCGFSFKTKSNLYKHRKSHAHRIKAGLASGMGGEMYPHGLEMERIPGEEFEEPTEGESTDSEEETSATSGHPAELSPRPKQPLLSSGLYSSGSHSSSHERCSLSQSSTAQSLEDPPPFVEPSSEHPLSHKPEDTHTIKQKLALRLSERKKVIDEQAFLSPGSKGSTESGYFSRSESAEQQVSPPNTNAKSYAEIIFGKCGRIGQRTAMLTATSTQPLLPLSTEDKPSLVPLSVPRTQVIEHITKLITINEAVVDTSEIDSVKPRRSSLSRRSSMESPKSSLYREPLSSHSEKTKPEQSLLSLQHPPSTAPPVPLLRSHSMPSAACTISTPHHPFRGSYSFDDHITDSEALSHSSHVFTSHPRMLKRQPAIELPLGGEYSSEEPGPSSKDTASKPSDEVEPKESELTKKTKKGLKTKGVIYECNICGARYKKRDNYEAHKKYYCSELQIAKPISAGTHTSPEAEKSQIEHEPWSQMMHYKLGTTLELTPLRKRRKEKSLGDEEEPPAFESTKSQFGSPGPSDAARNLPLESTKSPAEPSKSVPSLEGPTGFQPRTPKPGSGSESGKERRTTSKEISVIQHTSSFEKSDSLEQPSGLEGEDKPLAQFPSPPPAPHGRSAHSLQPKLVRQPNIQVPEILVTEEPDRPDTEPEPPPKEPEKTEEFQWPQRSQTLAQLPAEKLPPKKKRLRLAEMAQSSGESSFESSVPLSRSPSQESNVSLSGSSRSASFERDDHGKAEAPSPSSDMRPKPLGTHMLTVPSHHPHAREMRRSASEQSPNVSHSAHMTETRSKSFDYGSLSLTGPSAPAPVAPPARVAPPERRKCFLVRQASLSRPPESELEVAPKGRQESEEPQPSSSKPSAKSSLSQISSAATSHGGPPGGKGPGQDRPPLGPTVPYTEALQVFHHPVAQTPLHEKPYLPPPVSLFSFQHLVQHEPGQSPEFFSTQAMSSLLSSPYSMPPLPPSLFQAPPLPLQPTVLHPGQLHLPQLMPHPANIPFRQPPSFLPMPYPTSSALSSGFFLPLQSQFALQLPGDVESHLPQIKTSLAPLATGSAGLSPSTEYSSDIRLPPVAPPASSSAPTSAPPLALPACPDTMVSLVVPVRVQTNMPSYGSAMYTTLSQILVTQSQGSSATVALPKFEEPPSKGTTVCGADVHEVGPGPSGLSEEQSRAFPTPYLRVPVTLPERKGTSLSSESILSLEGSSSTAGGSKRVLSPAGSLELTMETQQQKRVKEEEASKADEKLELVKPCSVVLTSTEDGKRPEKSHLGNQGQGRRELEMLSSLSSDPSDTKEIPPLPHPALSHGTAPGSEALKEYPQPSGKPHRRGLTPLSVKKEDSKEQPDLPSLAPPSSLPLSETSSRPAKSQEGTDSKKVLQFPSLHTTTNVSWCYLNYIKPNHIQHADRRSSVYAGWCISLYNPNLPGVSTKAALSLLRSKQKVSKETYTMATAPHPEAGRLVPSSSRKPRMTEVHLPSLVSPEGQKDLARVEKEEERRGEPEEDAPASQRGEPARIKIFEGGYKSNEEYVYVRGRGRGKYVCEECGIRCKKPSMLKKHIRTHTDVRPYVCKHCHFAFKTKGNLTKHMKSKAHSKKCQETGVLEELEAEEGTSDDLFQDSEGREGSEAVEEHQFSDLEDSDSDSDLDEDEDEDEEESQDELSRPSSEAPPPGPPHALRADSSPILGPQPPDAPASGTEATRGSSVSEAERLTASSCSMSSQSMPGLPWLGPAPLGSVEKDTGSALSYKPVSPRRPWSPSKEAGSRPPLARKHSLTKNDSSPQRCSPAREPQASAPSPPGLHVDPGRGMGALPCGSPRLQLSPLTLCPLGRELAPRAHVLSKLEGTTDPGLPRYSPTRRWSPGQAESPPRSAPPGKWALAGPGSPSAGEHGPGLGLDPRVLFPPAPLPHKLLSRSPETCASPWQKAESRSPSCSPGPAHPLSSRPFSALHDFHGHILARTEENIFSHLPLHSQHLTRAPCPLIPIGGIQMVQARPGAHPTLLPGPTAAWVSGFSGGGSDLTGAREAQERGRWSPTESSSASVSPVAKVSKFTLSSELEGGDYPKERERTGGGPGRPPDWTPHGTGAPAEPTPTHSPCTPPDTLPRPPQGRRAAQSWSPRLESPRAPTNPEPSATPPLDRSSSVGCLAEASARFPARTRNLSGEPRTRQDSPKPSGSGEPRAHPHQPEDRVPPNA

SEQ ID NO: 772 - protein, 2405 aa, Homo sapiens
MDPEQSVKGTKKAEGSPRKRLTKGEAIQTSVSSSVPYPGSGTAATQESPAQELLAPQPFPGPSSVLREGSQEKTGQQQKPPKRPPIEASVHISQLPQHPLTPAFMSPGKPEHLLEGSTWQLVDPMRPGPSGSFVAPGLHPQSQLLPSHASIIPPEDLPGVPKVFVPRPSQVSLKPTEEAHKKERKPQKPGKYICQYCSRPCAKPSVLQKHIRSHTGERPYPCGPCGFSFKTKSNLYKHRKSHAHRIKAGLASGMGGEMYPHGLEMERIPGEEFEEPTEGESTDSEEETSATSGHPAELSPRPKQPLLSSGLYSSGSHSSSHERCSLSQSSTAQSLEDPPPFVEPSSEHPLSHKPEDTHTIKQKLALRLSERKKVIDEQAFLSPGSKGSTESGYFSRSESAEQQVSPPNTNAKSYAEIIFGKCGRIGQRTAMLTATSTQPLLPLSTEDKPSLVPLSVPRTQVIEHITKLITINEAVVDTSEIDSVKPRRSSLSRRSSMESPKSSLYREPLSSHSEKTKPEQSLLSLQHPPSTAPPVPLLRSHSMPSAACTISTPHHPFRGSYSFDDHITDSEALSHSSHVFTSHPRMLKRQPAIELPLGGEYSSEEPGPSSKDTASKPSDEVEPKESELTKKTKKGLKTKGVIYECNICGARYKKRDNYEAHKKYYCSELQIAKPISAGTHTSPEAEKSQIEHEPWSQMMHYKLGTTLELTPLRKRRKEKSLGDEEEPPAFESTKSQFGSPGPSDAARNLPLESTKSPAEPSKSVPSLEGPTGFQPRTPKPGSGSESGKERRTTSKEISVIQHTSSFEKSDSLEQPSGLEGEDKPLAQFPSPPPAPHGRSAHSLQPKLVRQPNIQVPEILVTEEPDRPDTEPEPPPKEPEKTEEFQWPQRSQTLAQLPAEKLPPKKKRLRLAEMAQSSGESSFESSVPLSRSPSQESNVSLSGSSRSASFERDDHGKAEAPSPSSDMRPKPLGTHMLTVPSHHPHAREMRRSASEQSPNVSHSAHMTETRSKSFDYGSLSLTGPSAPAPVAPPARVAPPERRKCFLVRQASLSRPPESELEVAPKGRQESEEPQPSSSKPSAKSSLSQISSAATSHGGPPGGKGPGQDRPPLGPTVPYTEALQVFHHPVAQTPLHEKPYLPPPVSLFSFQHLVQHEPGQSPEFFSTQAMSSLLSSPYSMPPLPPSLFQAPPLPLQPTVLHPGQLHLPQLMPHPANIPFRQPPSFLPMPYPTSSALSSGFFLPLQSQFALQLPGDVESHLPQIKTSLAPLATGSAGLSPSTEYSSDIRLPPVAPPASSSAPTSAPPLALPACPDTMVSLVVPVRVQTNMPSYGSAMYTTLSQILVTQSQGSSATVALPKFEEPPSKGTTVCGADVHEVGPGPSGLSEEQSRAFPTPYLRVPVTLPERKGTSLSSESILSLEGSSSTAGGSKRVLSPAGSLELTMETQQQKRVKEEEASKADEKLELVKPCSVVLTSTEDGKRPEKSHLGNQGQGRRELEMLSSLSSDPSDTKEIPPLPHPALSHGTAPGSEALKEYPQPSGKPHRRGLTPLSVKKEDSKEQPDLPSLAPPSSLPLSETSSRPAKSQEGTDSKKVLQFPSLHTTTNVSWCYLNYIKPNHIQHADRRSSVYAGWCISLYNPNLPGVSTKAALSLLRSKQKVSKETYTMATAPHPEAGRLVPSSSRKPRMTEVHLPSLVSPEGQKDLARVEKEEERRGEPEEDAPASQRGEPARIKIFEGGYKSNEEYVYVRGRGRGKYVCEECGIRCKKPSMLKKHIRTHTDVRPYVCKHCHFAFKTKGNLTKHMKSKAHSKKCQETGVLEELEAEEGTSDDLFQDSEGREGSEAVEEHQFSDLEDSDSDSDLDEDEDEDEEESQDELSRPSSEAPPPGPPHALRADSSPILGPQPPDAPASGTEATRGSSVSEAERLTASSCSMSSQSMPGLPWLGPAPLGSVEKDTGSALSYKPVSPRRPWSPSKEAGSRPPLARKHSLTKNDSSPQRCSPAREPQASAPSPPGLHVDPGRGMGALPCGSPRLQLSPLTLCPLGRELAPRAHVLSKLEGTTDPGLPRYSPTRRWSPGQAESPPRSAPPGKWALAGPGSPSAGEHGPGLGLDPRVLFPPAPLPHKLLSRSPETCASPWKAESRSPSCSPGPAHPLSSRPFSALHDFHGHILARTEENIFSHLPLHSQHLTRAPCPLIPIGGIQMVQARPGAHPTLLPGPTAAWVSGFSGGGSDLTGAREAQERGRWSPTESSSASVSPVAKVSKFTLSSELEGGDYPKERERTGGGPGRPPDWTPHGTGAPAEPTPTHSPCTPPDTLPRPPQGRRAAQSWSPRLESPRAPTNPEPSATPPLDRSSSVGCLAEASARFPARTRNLSGEPRTRQDSPKPSGSGEPRAHPHQPEDRVPPNA

SEQ ID NO: 773 - protein, 801 aa, Homo sapiens
MKTSPRRPLILKRRRLPLPVQNAPSETSEEEPKRSPAQQESNQAEASKEVAESNSCKFPAGIKIINHPTMPNTQVVAIPNNANIHSIITALTAKGKESGSSGPNKFILISCGGAPTQPPGLRPQTQTSYDAKRTEVTLETLGPKPAARDVNLPRPPGALCEQKRETCADGEAAGCTINNSLSNIQWLRKMSSDGLGSRSIKQEMEEKENCHLEQRQVKVEEPSRPSASWQNSVSERPPYSYMAMIQFAINSTERKRMTLKDIYTWIEDHFPYFKHIAKPGWKNSIRHNLSLHDMFVRETSANGKVSFWTIHPSANRYLTLDQVFKPLDPGSPQLPEHLESQQKRPNPELRRNMTIKTELPLGARRKMKPLLPRVSSYLVPIQFPVNQSLVLQPSVKVPLPLAASLMSSELARHSKRVRIAPKVFGEQVVFGYMSKFFSGDLRDFGTPITSLFNFIFLCLSVLLAEEGIAPLSSAGPGKEEKLLFGEGFSPLLPVQTIKEEEIQPGEEMPHLARPIKVESPPLEEWPSPAPSFKEESSHSWEDSSQSPTPRPKKSYSGLRSPTRCVSEMLVIQHRERRERSRSRRKQHLLPPCVDEPELLFSEGPSTSRWAAELPFPADSSDPASQLSYSQEVGGPFKTPIKETLPISSTPSKSVLPRTPESWRLTPPAKVGGLDFSPVQTSQGASDPLPDPLGLMDLSTTPLQSAPPLESPQRLLSSEPLDLISVPFGNSSPSDIDVPKPGSPEPQVSGLAANRSLTEGLVLDTMNDSLSKILLDISFPGLDEDPLGPDNINWSQFIPELQ

SEQ ID NO: 774 - protein, 748 aa, Homo sapiens
MKTSPRRPLILKRRRLPLPVQNAPSETSEEEPKRSPAQQESNQAEASKEVAESNSCKFPAGIKIINHPTMPNTQVVAIPNNANIHSIITALTAKGKESGSSGPNKFILISCGGAPTQPPGLRPQTQTSYDAKRTEVTLETLGPKPAARDVNLPRPPGALCEQKRETCADGEAAGCTINNSLSNIQWLRKMSSDGLGSRSIKQEMEEKENCHLEQRQVKVEEPSRPSASWQNSVSERPPYSYMAMIQFAINSTERKRMTLKDIYTWIEDHFPYFKHIAKPGWKNSIRHNLSLHDMFVRETSANGKVSFWTIHPSANRYLTLDQVFKQQKRPNPELRRNMTIKTELPLGARRKMKPLLPRVSSYLVPIQFPVNQSLVLQPSVKVPLPLAASLMSSELARHSKRVRIAPKVLLAEEGIAPLSSAGPGKEEKLLFGEGFSPLLPVQTIKEEEIQPGEEMPHLARPIKVESPPLEEWPSPAPSFKEESSHSWEDSSQSPTPRPKKSYSGLRSPTRCVSEMLVIQHRERRERSRSRRKQHLLPPCVDEPELLFSEGPSTSRWAAELPFPADSSDPASQLSYSQEVGGPFKTPIKETLPISSTPSKSVLPRTPESWRLTPPAKVGGLDFSPVQTSQGASDPLPDPLGLMDLSTTPLQSAPPLESPQRLLSSEPLDLISVPFGNSSPSDIDVPKPGSPEPQVSGLAANRSLTEGLVLDTMNDSLSKILLDISFPGLDEDPLGPDNINWSQFIPELQ

SEQ ID NO: 775 - protein, 763 aa, Homo sapiens
MKTSPRRPLILKRRRLPLPVQNAPSETSEEEPKRSPAQQESNQAEASKEVAESNSCKFPAGIKIINHPTMPNTQVVAIPNNANIHSIITALTAKGKESGSSGPNKFILISCGGAPTQPPGLRPQTQTSYDAKRTEVTLETLGPKPAARDVNLPRPPGALCEQKRETCADGEAAGCTINNSLSNIQWLRKMSSDGLGSRSIKQEMEEKENCHLEQRQVKVEEPSRPSASWQNSVSERPPYSYMAMIQFAINSTERKRMTLKDIYTWIEDHFPYFKHIAKPGWKNSIRHNLSLHDMFVRETSANGKVSFWTIHPSANRYLTLDQVFKPLDPGSPQLPEHLESQQKRPNPELRRNMTIKTELPLGARRKMKPLLPRVSSYLVPIQFPVNQSLVLQPSVKVPLPLAASLMSSELARHSKRVRIAPKVLLAEEGIAPLSSAGPGKEEKLLFGEGFSPLLPVQTIKEEEIQPGEEMPHLARPIKVESPPLEEWPSPAPSFKEESSHSWEDSSQSPTPRPKKSYSGLRSPTRCVSEMLVIQHRERRERSRSRRKQHLLPPCVDEPELLFSEGPSTSRWAAELPFPADSSDPASQLSYSQEVGGPFKTPIKETLPISSTPSKSVLPRTPESWRLTPPAKVGGLDFSPVQTSQGASDPLPDPLGLMDLSTTPLQSAPPLESPQRLLSSEPLDLISVPFGNSSPSDIDVPKPGSPEPQVSGLAANRSLTEGLVLDTMNDSLSKILLDISFPGLDEDPLGPDNINWSQFIPELQ

SEQ ID NO: 776 - protein, 206 aa, Homo sapiens
XAKPGWKNSIRHNLSLHDMFVRETSANGKVSFWTIHPSANRYLTLDQVFKPLDPGSPQLPEHLESQQQKRPNPELRRNMTIKTELPLGARRKMKPLLPRVSSYLVPIQFPVNQSLVLQPSVKVPLPLAASLMSSELARHSKRVRIAPKVLLAEEGIAPLSSAGPGKEEKLLFGEGFSPLLPVQTIKEEEIQPGEEMPHLARPIKVE

SEQ ID NO: 777 - protein, 80 aa, Homo sapiens
TALTAKGKESGSSGPNKFILISCGGAPTQPPGLRPQTQTSYDAKRTEVTLETLGPKPAARDVNLPRPPGALCEQKRETCG

SEQ ID NO: 778 - protein, 748 aa, Homo sapiens
MKTSPRRPLILKRRRLPLPVQNAPSETSEEEPKRSPAQQESNQAEASKEVAESNSCKFPAGIKIINHPTMPNTQVVAIPNNANIHSIITALTAKGKESGSSGPNKFILISCGGAPTQPPGLRPQTQTSYDAKRTEVTLETLGPKPAARDVNLPRPPGALCEQKRETCDGEAAGCTINNSLSNIQWLRKMSSDGLGSRSIKQEMEEKENCHLEQRQVKVEEPSRPSASWQNSVSERPPYSYMAMIQFAINSTERKRMTLKDIYTWIEDHFPYFKHIAKPGWKNSIRHNLSLHDMFVRETSANGKVSFWTIHPSANRYLTLDQVFKQQQKRPNPELRRNMTIKTELPLGARRKMKPLLPRVSSYLVPIQFPVNQSLVLQPSVKVPLPLAASLMSSELARHSKRVRIAPKVLLAEEGIAPLSSAGPGKEEKLLFGEGFSPLLPVQTIKEEEIQPGEEMPHLARPIKVESPPLEEWPSPAPSFKEESSHSWEDSSQSPTPRPKKSYSGLRSPTRCVSEMLVIQHRERRERSRSRRKQHLLPPCVDEPELLFSEGPSTSRWAAELPFPADSSDPASQLSYSQEVGGPFKTPIKETLPISSTPSKSVLPRTPESWRLTPPAKVGGLDFSPVQTSQGASDPLPDPLGLMDLSTTPLQSAPPLESPQRLLSSEPLDLISVPFGNSSPSDIDVPKPGSPEPQVSGLAANRSLTEGLVLDTMNDSLSKILLDISFPGLDEDPLGPDNINWSQFIPELQ

SEQ ID NO: 779 - protein, 432 aa, Homo sapiens
MSELKDCPLQFHDFKSVDHLKVCPRYTAVLARSEDDGIGIEELDTLQLELETLLSSASRRLRVLEAETQILTDWQDKKGDRRFLKLGRDHELGAPPKHGKPKKQKLEGKAGHGPGPGPGRPKSKNLQPKIQEYEFTDDPIDVPRIPKNDAPNRFWASVEPYCADITSEEVRTLEELLKPPEDEAEHYKIPPLGKHYSQRWAQEDLLEEQKDGARAAAVADKKKGLMGPLTELDTKDVDALLKKSEAQHEQPEDGCPFGALTQRLLQALVEENIISPMEDSPIPDMSGKESGADGASTSPRNQNKPFSVPHTKSLESRIKEELIAQGLLESEDRPAEDSEDEVLAELRKRQAELKALSAHNRTKKHDLLRLAKEEVSRQELRQRVRMADNEVMDAFRKIMAARQKKRTPTKKEKDQAWKTLKERESILKLLDG

SEQ ID NO: 780 - protein, 432 aa, Homo sapiens; identical to SEQ ID NO: 779

SEQ ID NO: 781 - protein, 369 aa, Homo sapiens
MSELKDCPLQFHDFKSVDHLKVCPRYTAVLARSEDDGIGIEELDTLQLELETLLSSASRRLRVLEAETQILTDWQDKKGDRRFLKLGRDHELGAPPKHGKPKKQKLEGKAGHGPGPGPGRPKSKNLQPKIQEYEFTDDPIDVPRIPKNDAPNRFWASVEPYCADITSEEVRTLEELLKPPEDEAEHYKIPPLGKHYSQRWAQEDLLEEQKDGARAAAVADKKKGLMGPLTELDTKDVDALLKKSEAQHEQPEDGCPFGALTQRLLQALVEENIISPMEDSPIPDMSGKESGADGASTSPRNQNKPFSVPHTKSLESRIKEELIAQGLLESEDRPAEDSEDEVLAELRKRQAELKALSAHNRTKKHDLLR

SEQ ID NO: 782 - protein, 95 aa, Homo sapiens
MSELKDCPLQFHDFKSVDHLKVCPRYTAVLARSEDDGIGIEELDTLQLELETLLSSASRRLRVLEAETQILTDWQDKKGDRRFLKLGRDHELGAP

SEQ ID NO: 783 - protein, 99 aa, Homo sapiens
MPSDFISLLSADLDLESPKSLYSRDSLKLHPSQNFHRAGLLEESVYDLLPKELQLPPSRETSVASMSQTSGGEAGSPPPAVVAAGFASEAGSVCIKNDL

SEQ ID NO: 784 - protein, 1531 aa, Homo sapiens
MPSDFISLLSADLDLESPKSLYSRESVYDLLPKELQLPPSRETSVASMSQTSGGEAGSPPPAVVAADASSAPSSSSMGGACSSFTTSSSPTIYSTSVTDSKAMQVESCSSAVGVSNRGVSEKQLTSNTVQQHPSTPKRHTVLYISPPPEDLLDNSRMSCQDEGCGLESEQSCSMWMEDSPSNFSNMSTSSYNDNTEVPRKSRKRNPKQRPGVKRRDCEESNMDIFDADSAKAPHYVLSQLTTDNKGNSKAGNGTLENQKGTGVKKSPMLCGQYPVKSEGKELKIVVQPETQHRARYLTEGSRGSVKDRTQQGFPTVKLEGHNEPVVLQVFVGNDSGRVKPHGFYQACRVTGRNTTPCKEVDIEGTTVIEVGLDPSNNMTLAVDCVGILKLRNADVEARIGIAGSKKKSTRARLVFRVNIMRKDGSTLTLQTPSSPILCTQPAGVPEILKKSLHSCSVKGEEEVFLIGKNFLKGTKVIFQENVSDENSWKSEAEIDMELFHQNHLIVKVPPYHDQHITLPVSVGIYVVTNAGRSHDVQPFTYTPDPAAAGALNVNVKKEISSPARPCSFEEAMKAMKTTGCNLDKVNIIPNALMTPLIPSSMIKSEDVTPMEVTAEKRSSTIFKTTKSVGSTQQTLENISNIAGNGSFSSPSSSHLPSENEKQQQIQPKAYNPETLTTIQTQDISQPGTFPAVSASSQLPNSDALLQQATQFQTRETQSREILQSDGTVVNLSQLTEASQQQQQSPLQEQAQTLQQQISSNIFPSPNSVSQLQNTIQQLQAGSFTGSTASGSSGSVDLVQQVLEAQQQLSSVLFSAPDGNENVQEQLSADIFQQVSQIQSGVSPGMFSSTEPTVHTRPDNLLPGRAESVHPQSENTLSNQQQQQQQQQQVMESSAAMVMEMQQSICQAAAQIQSELFPSTASANGNLQQSPVYQQTSHMMSALSTNEDMQMQCELFSSPPAVSGNETSTTTTQQVATPGTTMFQTSSSGDGEETGTQAKQIQNSVFQTMVQMQHSGDNQPQVNLFSSTKSMMSVQNSGTQQQGNGLFQQGNEMMSLQSGNFLQQSSHSQAQLFHPQNPIADAQNLSQETQGSLFHSPNPIVHSQTSTTSSEQMQPPMFHSQSTIAVLQGSSVPQDQQSTNIFLSQSPMNNLQTNTVAQEAFFAAPNSISPLQSTSNSEQQAAFQQQAPISHIQTPMLSQEQAQPPQQGLFQPQVALGSLPPNPMPQSQQGTMFQSQHSIVAMQSNSPSQEQQQQQQQQQQQQQQQQQSILFSNQNTMATMASPKQPPPNMIFNPNQNPMANQEQQNQSIFHQQSNMAPMNQEQQPMQFQSQSTVSSLQNPGPTQSESSQTPLFHSSPQIQLVQGSPSSQEQQVTLFLSPASMSALQTSINQQDMQQSPLYSPQNNMPGIQGATSSPQPQATLFHNTAGGTMNQLQNSPGSSQQTSGMFLFGIQNNCSQLLTSGPATLPDQLMAISQPGQPQNEGQPPVTTLLSQQMPENSPLASSINTNQNIEKIDLLVSLQNQGNNLTGSF

SEQ ID NO: 785 - protein, 119 aa, Homo sapiens; identical to SEQ ID NO: 160

SEQ ID NO: 786 - protein, 1548 aa, Homo sapiens
MPSDFISLLSADLDLESPKSLYSRDSLKLHPSQNFHRAGLLEESVYDLLPKELQLPPSRETSVASMSQTSGGEAGSPPPAVVAADASSAPSSSSMGGACSSFTTSSSPTIYSTSVTDSKAMQVESCSSAVGVSNRGVSEKQLTSNTVQQHPSTPKRHTVLYISPPPEDLLDNSRMSCQDEGCGLESEQSCSMWMEDSPSNFSNMSTSSYNDNTEVPRKSRKRNPKQRPGVKRRDCEESNMDIFDADSAKAPHYVLSQLTTDNKGNSKAGNGTLENQKGTGVKKSPMLCGQYPVKSEGKELKIVVQPETQHRARYLTEGSRGSVKDRTQQGFPTVKLEGHNEPVVLQVFVGNDSGRVKPHGFYQACRVTGRNTTPCKEVDIEGTTVIEVGLDPSNNMTLAVDCVGILKLRNADVEARIGIAGSKKKSTRARLVFRVNIMRKDGSTLTLQTPSSPILCTQPAGVPEILKKSLHSCSVKGEEEVFLIGKNFLKGTKVIFQENVSDENSWKSEAEIDMELFHQNHLIVKVPPYHDQHITLPVSVGIYVVTNAGRSHDVQPFTYTPDPAAGALNVNVKKEISSPARPCSFEEAMKAMKTTGCNLDKVNIIPNALMTPLIPSSMIKSEDVTPMEVTAEKRSSTIFKTTKSVGSTQQTLENISNIAGNGSFSSPSSSHLPSENEKQQQIQPKAYNPETLTTIQTQDISQPGTFPAVSASSQLPNSDALLQQATQFQTRETQSREILQSDGTVVNLSQLTEASQQQQQSPLQEQAQTLQQQISSNIFPSPNSVSQLQNTIQQLQAGSFTGSTASGSSGSVDLVQQVLEAQQQLSSVLFSAPDGNENVQEQLSADIFQQVSQIQSGVSPGMFSSTEPTVHTRPDNLLPGRAESVHPQSENTLSNQQQQQQQQQQVMESSAAMVMEMQQSICQAAAQIQSELFPSTASANGNLQQSPVYQQTSHMMSALSTNEDMQMQCELFSSPPAVSGNETSTTTTQQVATPGTTMFQTSSSGDGEETGTQAKQIQNSVFQTMVQMQHSGDNQPQVNLFSSTKSMMSVQNSGTQQQGNGLFQQGNEMMSLQSGNFLQQSSHSQAQLFHPQNPIADAQNLSQETQGSLFHSPNPIVHSQTSTTSSEQMQPPMFHSQSTIAVLQGSSVPQDQQSTNIFLSQSPMNNLQTNTVAQEAFFAAPNSISPLQSTSNSEQQAAFQQQAPISHIQTPMLSQEQAQPPQQGLFQPQVALGSLPPNPMPQSQQGTMFQSQHSIVAMQSNSPSQEQQQQQQQQQQQQQQQQQSILFSNQNTMATMASPKQPPPNMIFNPNQNPMANQEQQNQSIFHQQSNMAPMNQEQQPMQFQSQSTVSSLQNPGPTQSESSQTPLFHSSPQIQLVQGSPSSQEQQVTLFLSPASMSALQTSINQQDMQQSPLYSPQNNMPGIQGATSSPQPQATLFHNTAGGTMNQLQNSPGSSQQTSGMFLFGIQNNCSQLLTSGPATLPDQLMAISQPGQPQNEGQPPVTTLLSQQMPENSPLASSINTNQNIEKIDLLVSLQNQGNNLTGSF

SEQ ID NO: 787 - protein, 1549 aa, Homo sapiens
MPSDFISLLSADLDLESPKSLYSRDSLKLHPSQNFHRAGLLEESVYDLLPKELQLPPSRETSVASMSQTSGGEAGSPPPAVVAADASSAPSSSSMGGACSSFTTSSSPTIYSTSVTDSKAMQVESCSSAVGVSNRGVSEKQLTSNTVQQHPSTPKRHTVLYISPPPEDLLDNSRMSCQDEGCGLESEQSCSMWMEDSPSNFSNMSTSSYNDNTEVPRKSRKRNPKQRPGVKRRDCEESNMDIFDADSAKAPHYVLSQLTTDNKGNSKAGNGTLENQKGTGVKKSPMLCGQYPVKSEGKELKIVVQPETQHRARYLTEGSRGSVKDRTQQGFPTVKLEGHNEPVVLQVFVGNDSGRVKPHGFYQACRVTGRNTTPCKEVDIEGTTVIEVGLDPSNNMTLAVDCVGILKLRNADVEARIGIAGSKKKSTRARLVFRVNIMRKDGSTLTLQTPSSPILCTQPAGVPEILKKSLHSCSVKGEEEVFLIGKNFLKGTKVIFQENVSDENSWKSEAEIDMELFHQNHLIVKVPPYHDQHITLPVSVGIYVVTNAGRSHDVQPFTYTPDPAAAGALNVNVKKEISSPARPCSFEEAMKAMKTTGCNLDKVNIIPNALMTPLIPSSMIKSEDVTPMEVTAEKRSSTIFKTTKSVGSTQQTLENISNIAGNGSFSSPSSSHLPSENEKQQQIQPKAYNPETLTTIQTQDISQPGTFPAVSASSQLPNSDALLQQATQFQTRETQSREILQSDGTVVNLSQLTEASQQQQQSPLQEQAQTLQQQISSNIFPSPNSVSQLQNTIQQLQAGSFTGSTASGSSGSVDLVQQVLEAQQQLSSVLFSAPDGNENVQEQLSADIFQQVSQIQSGVSPGMFSSTEPTVHTRPDNLLPGRAESVHPQSENTLSNQQQQQQQQQQVMESSAAMVMEMQQSICQAAAQIQSELFPSTASANGNLQQSPVYQQTSHMMSALSTNEDMQMQCELFSSPPAVSGNETSTTTTQQVATPGTTMFQTSSSGDGEETGTQAKQIQNSVFQTMVQMQHSGDNQPQVNLFSSTKSMMSVQNSGTQQQGNGLFQQGNEMMSLQSGNFLQQSSHSQAQLFHPQNPIADAQNLSQETQGSLFHSPNPIVHSQTSTTSSEQMQPPMFHSQSTIAVLQGSSVPQDQQSTNIFLSQSPMNNLQTNTVAQEAFFAAPNSISPLQSTSNSEQQAAFQQQAPISHIQTPMLSQEQAQPPQQGLFQPQVALGSLPPNPMPQSQQGTMFQSQHSIVAMQSNSPSQEQQQQQQQQQQQQQQQQQSILFSNQNTMATMASPKQPPPNMIFNPNQNPMANQEQQNQSIFHQQSNMAPMNQEQQPMQFQSQSTVSSLQNPGPTQSESSQTPLFHSSPQIQLVQGSPSSQEQQVTLFLSPASMSALQTSINQQDMQQSPLYSPQNNMPGIQGATSSPQPQATLFHNTAGGTMNQLQNSPGSSQQTSGMFLFGIQNNCSQLLTSGPATLPDQLMAISQPGQPQNEGQPPVTTLLSQQMPENSPLASSINTNQNIEKIDLLVSLQNQGNNLTGSF

SEQ ID NO: 788 - protein, 1455 aa, Homo sapiens; identical to SEQ ID NO: 742

SEQ ID NO: 789 - protein, 1455 aa, Homo sapiens; identical to SEQ ID NO: 742

SEQ ID NO: 790 - protein, 141 aa, Homo sapiens
AMQVESCSSAVGNNMPGIQGATSSPQPQATLFHNTAGGTMNQLQNSPGSSQQTSGMFLFGIQNNCSQLLTSGPATLPDQLMAISQPGQPQNEGQPPVTTLLSQQMPENSPLASSINTNQNIEKIDLLVSLQNQGNNLTGSF

SEQ ID NO: 791 - protein, 143 aa, Homo sapiens
XTLENQKGTGVKKSPMLCGQYPVKSEGKELKIVVQPETQHRARYLTEGSRGSVKDRTQQGFPTVKLEGHNEPVVLQVFVGNDSGRVKPHGFYQACRVTGRNTTPCKEVDIEGTTVIEVGLDPSNNMTLASASRSARNLKEKLA